(19) United States
(12) Patent Application Publication
(10) Pub. No.: US 2025/0281557 A1
(43) Pub. Date: Sep. 11, 2025

(54) METHODS FOR CASCADE AMPLIFICATIONS OF THERAPEUTIC PAYLOADS (CATP) & COMPOSITIONS FOR CANCER IMMUNOTHERAPIES AND GENE THERAPY

(71) Applicant: SunVax mRNA Therapeutics Inc., Beverly, MA (US)

(72) Inventors: Yingzhong LI, Reading, MA (US); Libin ZHANG, Lynnfield, MA (US)

(73) Assignee: SunVax mRNA Therapeutics Inc., Beverly, MA (US)

(21) Appl. No.: 19/074,698

(22) Filed: Mar. 10, 2025

Related U.S. Application Data
(60) Provisional application No. 63/563,062, filed on Mar. 8, 2024.
(60) Provisional application No. 63/738,692, filed on Dec. 24, 2024.

(51) Int. Cl. A61K 35/768; A61K 38/16; A61K 38/20; A61K 48/00; A61P 35/00

(52) U.S. Cl. CPC A61K 35/768; A61K 38/162; A61K 38/20; A61K 38/208; A61K 48/0033; A61K 48/0066; A61P 35/00; C12N 2770/36122; C12N 2770/36132; C12N 2770/36162; C12N 2770/36171

(57) ABSTRACT

The invention relates to compositions and methods for the preparation, manufacture and therapeutic use of oncolytic defective virus compositions and methods of in vivo synthesis thereof. The composition includes a first nucleic acid construct encoding a self-amplifying mRNA (sa-mRNA) encoding at least one gene of interest (GOI) or a plurality of GOIs, a second nucleic acid construct encoding an mRNA encoding at least one virus structural protein, and at least one payload delivery system.

### CROSS-REFERENCE TO RELATED APPLICATIONS

This application claims priority benefit of U.S. Provisional Appl. No. 63/563,062 filed on 8 Mar. 2024 and U.S. Provisional Appl. No. 63/738,692 filed on 24 Dec. 2024, the disclosures of each of which are incorporated herein by reference in their entireties.

### SEQUENCE LISTING STATEMENT

The instant application contains a Sequence Listing in electronic format which has been submitted electronically. Said Sequence Listing, created on 25 Feb. 2025, is named “5292-114PCT-ST26.xml” and is 444,100 bytes in size. The information in the electronic format of the Sequence Listing is part of the present application and is incorporated herein by reference in its entirety.

### TECHNICAL FIELD

The present disclosure provides oncolytic defective virus compositions and methods of synthesis thereof, for use in cancer immunotherapies.

### BACKGROUND

It is a challenge in the field of therapeutics to design, synthesize and deliver a therapeutic agent, e.g., a ribonucleic acid (RNA) for example, a messenger RNA (mRNA) or self-amplifying mRNA (sa-mRNA) inside a cell in vivo, such as to effect physiologic outcomes which are beneficial to the cell, tissue or organ and ultimately to an organism.

In particular, current cancer treatments, including existing viral vectors, chemotherapy, radiation, and surgery, lack the specificity to selectively treat cancerous cells, while maintaining the health and viability of normal, non-cancerous cells, and can produce undesirable off-target effects. As such, there is a need in the art for cancer therapies that are broadly efficacious in multiple cancers, are capable of selectively eliminating cancerous cells and produce less undesirable off-target effects.

While the use of oncolytic viruses has been contemplated, a principal danger of the use of viruses in cancer treatment is the possibility of dissemination of a wild-type virus reconstituted in the patient's healthy cells or tissues. Oncolytic viruses are capable of selective replication in dividing cells (e.g., cancer cells) while leaving non-dividing cells (e.g., healthy cells) unharmed. As the infected dividing cells are destroyed by lysis, they release new infectious virus particles to infect the surrounding dividing cells. Cancer cells are ideal hosts for many viruses because they have the antiviral interferon pathway inactivated or have mutated tumor suppressor genes that enable viral replication to proceed unhindered. Use of oncolytic viruses carries the risk of non-specific viral infection of healthy cells, leading to the death of non-cancerous cells and tissues.

Moreover, achieving sufficient production of oncolytic virus therapies in vitro remains difficult because (1) in vitro manufacturing processes need to be established for each OV individually; (2) different virus features-particle size, presence/absence of an envelope, and host species-require specific requirements to ensure sterility, for handling, and for toxicity testing; and (2) optimization of serum-free culture conditions, increasing virus yields, development of scalable purification strategies, and formulations guaranteeing long-term stability require further optimization and characterization (Ungerechts et al., 2006, Mol Ther Methods Clin Dev 3: 16018).

Nucleic acid therapies, such as mRNA therapies, are able to deliver one or more gene or genes of interest that are encoded by a nucleic acid to a subject in need thereof. However, achieving adequate gene expression is a medical challenge for nucleic acid therapies because the number of RNA transcripts available in vivo is proportional to the number of nucleic acid molecules successfully delivered during administration, thus existing nucleic acid therapies may require large doses or repeated administrations. Large doses and repeated administrations of nucleic acid therapies can elicit undesirable immune responses and repeated administration can render subsequent administration of the same therapeutic less effective.

### SUMMARY OF THE INVENTION

The present disclosure includes a composition comprising: a first nucleic acid construct encoding a self-amplifying mRNA (sa-mRNA) encoding at least one gene of interest (GOI) or a plurality of GOIs; a second nucleic acid construct encoding an mRNA encoding at least one virus structural protein; and at least one payload delivery system, wherein the at least one payload delivery system is a non-viral payload delivery system and wherein the payload is at least one nucleic acid construct.

In one aspect, the composition comprises a first nucleic acid construct encoding a self-amplifying mRNA (sa-mRNA) comprising SEQ ID NO: 45 and at least one of SEQ ID NOs: 54, 101 or 102; a second nucleic acid construct encoding an mRNA encoding at least one virus structural protein from SFV4; and at least one payload delivery system, wherein the non-viral payload delivery system is an LNP, wherein the LNP comprises the compound (IVe), wherein the payload is at least one nucleic acid construct.

In some aspects, the at least one virus structural protein encodes a viral capsid protein, a viral envelope protein, or a combination thereof. In some aspects, the first nucleic acid construct is or encodes a sa-mRNA molecule. In some aspects, the second nucleic acid construct is or encodes a mRNA molecule. In some aspects, the non-viral payload delivery system is an LNP. In some aspects, the LNP comprises an ionizable lipid, a phospholipid, a steroid, a polyethylene glycol (PEG) lipid, a modular lipid, or a combination thereof.

In on aspect, the first nucleic acid construct comprises an operably linked nucleic acid sequence comprising from 5′ to 3′:
[5′ UTR]-[nsP 1-4]-[SGP]-[GOI]-[3′ UTR]-[Poly A]
wherein, 5′ UTR is a 5′ untranslated region, nsP is a plurality of non-structural replicase domain sequences, SGP is a subgenomic promoter, GOI is one gene of interest or a plurality of genes of interest, 3′ UTR is a 3′ untranslated region, and poly A is a poly A tail. In some aspects, the first nucleic construct comprise more than one GOI operably linked to one or more SGP. In some aspects, each GOI is linked to a different SGP.

In some aspects, the at least one virus structural protein encoded by the second nucleic acid construct is from an alphavirus, adenovirus, a HSV, an echovirus, a polio virus, a vaccinia virus, a measles virus, a vesicular stomatitis, an orthomyxovirus, a parvovirus, a maraba virus, a coxsackievirus, an autonomous parvovirus, a myxoma virus, a Newcastle disease virus, a reovirus, a seneca valley virus morbillivirus virus, a retrovirus, an influenza virus, a sindbis virus, semliki forest virus, venezuelan equine encephalitis virus, or a poxvirus.

In one aspect, the present disclosure includes a pharmaceutical composition comprising: composition of the disclosure and a pharmaceutically acceptable carrier. In some aspects, the first nucleic acid construct and the second nucleic acid construct are present in the pharmaceutical composition in a 1:100 to 100:1 ratio. In some aspects, the first nucleic acid construct and the second nucleic acid construct are present in the pharmaceutical composition in a 50:1 to 1:50, 40:1 to 1:40, 30:1 to 1:30, 25:1 to 1:25, 20:1 to 1:20, 15:1 to 1:15, 10:1 to 1:10, 9:1 to 1:9, 8:1 to 1:8, 7:1 to 1:7, 7:1 to 1:7, 6:1 to 1:6, 5:1 to 1:5, 4:1 to 1:4, 3:1 to 1:3, 2:1 to 1:1, or 1:1 ratio.

In one aspect, the present disclosure provides a method of treating a subject having a tumor or cancer, comprising administering to a subject the pharmaceutical composition of the disclosure. In some aspects, the method induces apoptosis of a cancerous cell or tumor, the method comprising contacting the cancerous cell in vivo with the pharmaceutical composition of the disclosure such that the first nucleic acid construct and at least one virus structural protein replicate within said cancerous cell, to express the GOI, wherein the first nucleic acid replication, expression of the GOI and the at least one virus structural protein replication within the cancerous cell results in cell death.

In one aspect, the present disclosure provides method of increasing expression of a polypeptide encoded by a GOI in a cell comprising contacting the cell with the pharmaceutical composition of the disclosure such that the first nucleic acid construct and the second nucleic construct produce the at least one virus structural protein and the sa-mRNA within said cell, and the first nucleic acid construct expresses the GOI within said cell, wherein the at least one virus structural protein form a pseudoviral particle that encapsulate one or more of the sa-mRNA, and wherein expression of the polypeptide is increased by 50-fold, 100-fold, 200-fold, 300-fold, 400-fold, 500-fold compared to contacting the cell with the pharmaceutical composition in the absence of the second nucleic acid construct.

In one aspect, the present disclosure provides a method of increasing central memory CD8 T cell in a tumor draining lymph node or spleen comprising contacting a tumor with the pharmaceutical composition of the disclosure such that the first nucleic acid construct and the second nucleic construct produce the at least one virus structural protein and the sa-mRNA within said tumor to produce the at least one virus structural protein and the sa-mRNA, and the first nucleic acid construct expresses the GOI within said tumor, wherein the at least one virus structural protein form a pseudoviral particle, which encapsulate one or more of the sa-mRNA, and wherein central memory CD8 T cell count is increased by 5-fold, 6-fold, 7-fold, 8-fold, 9-fold, 10-fold, or more compared to contacting the cell with the pharmaceutical composition in the absence of the second nucleic acid construct.

A method of treating is disclosed herein for a subject having a tumor or cancer, comprising administering to a subject a composition comprising a nucleic acid construct encoding a self-amplifying mRNA (sa-mRNA) comprising SEQ ID NO: 45 and at least one of SEQ ID NOs: 54, 101 or 102.

Each of the aspects of the present disclosure can encompass various elements of the present disclosure. It is, therefore, anticipated that each of the aspects of the present disclosure involving any one element or combinations of elements can be included in each aspect of the present disclosure. This disclosure is not limited in its application to the details of construction and the arrangement of components set forth in the following detailed description or illustrated in the drawings.

### BRIEF DESCRIPTION OF THE DRAWINGS

FIG. 1A shows a diagram of introducing one embodiment of the composition of the present disclosure to a host cell, the result of the expression of the payload of the composition in the host cell, and the effect of the expression of the payload of the composition on the host cell and adjacent cells. FIG. 1B shows a schematic of the cascade amplification of therapeutic payload process. The production of defective viruses enables the infections of adjacent cells, further amplifying the therapeutic payloads and enhancing overall efficacy.

FIG. 2A shows the oncolytic effects of the composition of the present disclosure in HEK293 cells in vitro. FIG. 2B shows the same effects in APRE-19 cells in vitro.

FIGS. 3A-3B show the oncolytic effects of the composition of the present disclosure in C57B6L mice with B16F10 melanoma in vivo. FIG. 3A shows a diagram of the treatment plan.

FIG. 3B shows mIL12 expression in blood serum and tumor of the C57B6L mice with B16F10 melanoma.

FIGS. 4A-4C show the effects of the composition of the present disclosure in the central memory CD8 T cells, in the tumor draining lymph node (TDLN) and the effector CD8 T cells in the TDLN and spleen. The statistical analysis were performed by 2-way ANNOVA. “***” means p-value smaller than 0.001. FIG. 4A shows a diagram of the treatment plan. Six to eight weeks old C57B6L mice (5 replicates in each group) were subcutaneously inoculated with 1 million B16F10 melanoma cells. FIG. 4B shows the number of central memory (CD62L+ CD122+) and effector (CD62L− KLRG1+) CD8 T cells in the TDLN. FIG. 4C shows the number of central memory (CD62L+ CD122+) and effector (CD62L− KLRG1+) CD8 T cells in the spleen.

FIGS. 5A-5D show the oncolytic effects of the composition of the present disclosure in C57B6L mice with B16F10 melanoma in vivo. The statistical analysis were performed by 2-way ANNOVA. “***” means p-value smaller than 0.001. FIG. 5A shows a diagram of the treatment plan. FIG. 5B shows tumor area of the B16F10 melanoma (Y-Axis) versus days post intratumorally injection (X-Axis) in the treated mice. FIG. 5C shows the survival curve of the treated mice. FIG. 5D shows body weight changes of the treated mice (Y-Axis) versus days post intratumoral injection (X-Axis).

FIG. 6 shows the oncolytic effects of the composition of the present disclosure in C57B6L mice with MC38 in vivo. The statistical analysis were performed by 2-way ANNOVA.

FIG. 7 shows a schematic representation of a linearized SAM002 that is used as a template for production of sa-mRNA. The definitions of the abbreviations in the nucleotide sequence map are as follows: 5UTR is a 5′ untranslated region, nsP is a plurality of non-structural replicase domain sequences, SGP is a subgenomic promoter, Puromycin R is the puromycin resistance gene, 3′ UTR is a 3′ untranslated region and contigs are subgenomic intervals generated as vectors to facilitate sequencing and numbered for the identification of mutations after directed evolution.

FIG. 8 shows survival rates (Y-axis) versus days post-B16F10 cell inoculation (X-axis) treated by 10 μg SamRNA encoding with mIL-12 and 1 μg modified mRNA encoding with VEE, SFV4 or SIN groups. The P-Values were determined by a Comparison of Survival Curves (Kaper-myer) test.

FIGS. 9A-9D show the optimization of therapeutic payloads of CATP and mechanism of long-term immune memory by CATP. FIG. 9A shows the tumor area (Y-axis) versus days post-B16F10 cell inoculation (X-axis) and surivival rate (Y-axis) versus days post-B16F10 cell. FIG. 9B shows body weight changes (Y-axis) versus days post-B16F10 cell inoculation (X-axis). FIG. 9C shows inoculation (X-axis) and survival rates (Y-axis) versus days post-B16F10 cell. FIG. 9D shows re-challenges of tumor free mice in the group LNP encapsulated 5 μg of SamRNA encoding with mouse mutant IL-18 plus 5 μg of SamRNA encoding mouse IL-12 and 1 μg of modified mRNA encoding with SFV4 capsids/envelop proteins. The cured mice (n=10) and naïve mice (n=7) matched with age and sex and were challenged with 0.1 million B16F10 cells. Results are shown as survival rate (Y-axis) versus days post-B16F10 cell inoculation (X-axis).

FIGS. 10A-C show schemes of CATP by different viral envelops and capsids. FIG. 10A shows illustrations of constructs for CATP. FIG. 10B-10C show a comparison of therapeutic efficacies by CATP with VEE, SIN, and SFV4 capsids/envelop. Shown are tumor area (Y-axis) versus days post cancer cell inoculation (X-axis). FIG. 10B shows body weight changes (Y-axis) versus days post cancer cell inoculation (X-axis). FIG. 10C shows p-values labeled, which was determined by two-way ANOVA test.

FIGS. 11A-C show the optimization of therapeutic payloads of CATP in MC38 colon cancer model. The P-Values labeled was determined by a two-way ANOVA test or Comparison of Survival Curves (Kaper-myer) test. FIG. 11A shows the tumor area (Y-axis) versus days post MC38 cancer cell inoculation (X-axis). FIG. 11B shows the survival rate (Y-axis) versus days post MC38 cancer cell inoculation (X-axis). FIG. 11C shows the body weight changes (Y-axis) versus days post MC38 cancer cell inoculation (X-axis).

FIGS. 12A-12E show the therapeutic efficacy of CATP with optimized therapeutic payloads and SFV4 capsids in CT26 colon cancer and KPC (P53^{null} KRas^{G12D}) pancreatic duct cancer model. The P-Values labeled was determined by a two-way ANOVA test or Comparison of Survival Curves (Kaper-myer) test. FIGS. 12A-12C used six- to eight-week-old Balb/c mice. FIGS. 12D-12E used C57BL/6 mice (n=5 per group). FIG. 12A and FIG. 12D show tumor area (Y-axis) versus days post cancer cell inoculation (X-axis). FIG. 12B show survival rate (Y-axis) versus days post cancer cell inoculation (X-axis). FIGS. 12C and 12E show body weight changes (Y-axis) versus days post cancer cell inoculation (X-axis).

FIGS. 13A-13C show CATP with mouse IL-12 and mutant IL-18 forms long-term memory against tumor recurrences indicated by re-challenges of tumor free mice. The P-Values labeled was determined by a two-way ANOVA test or Comparison of Survival Curves (Kaper-myer) test. FIG. 13A shows the survival rate (Y-axis) versus days post rechallenges of MC38 cancer cell (X-axis). FIG. 13B shows the tumor area (Y-axis) versus days post-inoculation of tumor cells B16F10 cells (X-axis). FIG. 13C shows the tumor area (Y-axis) versus days post-inoculation of tumor cells MC38 cells (X-axis).

Note that any one of more of the illustrative components of the molecules and methods are optional and the present disclosure includes aspects that contain fewer than all of the illustrated elements.

### DETAILED DESCRIPTION

The disclosure relates to novel compositions, and methods for the preparation, manufacture and therapeutic use thereof. In one aspect, the present disclosure provides a composition comprising: a first nucleic acid construct encoding a self-amplifying mRNA (sa-mRNA) encoding at least one gene of interest (GOI) or a plurality of GOIs; a second nucleic acid construct encoding an mRNA encoding at least one virus structural protein; and at least one payload delivery system, wherein the at least one payload delivery system is a non-viral payload delivery system and wherein the payload is at least one nucleic acid construct. In some aspects, the at least one virus structural protein encodes a viral capsid protein, viral envelope protein, or a combination thereof. In some aspects, the present disclosure provides pharmaceutical compositions comprising the composition of the disclosure and methods of treating a proliferative disease.

It is important to note that while many of the approaches described in this specification and the examples given are focused on cancer treatment, they are equally applicable to other uses, such as for vaccine development, gene therapy or gene regulation.

Although the present disclosure is described in detail below, it is to be understood that this disclosure is not limited to the particular methodologies, protocols and reagents described herein as these may vary. It is also to be understood that the terminology used herein is for the purpose of describing particular aspects only, and is not intended to limit the scope of the present disclosure. Unless defined otherwise, all technical and scientific terms used herein have the same meanings as commonly understood by one of ordinary skill in the art.

In one aspect, the present disclosure provides constructs of the sa-mRNAs and variations thereof as shown and described, and methods of making and using the constructs. The present disclosure includes noncytopathic and cytopathic versions of the sa-mRNAs and variations thereof. The present disclosure includes sequences and engineering of conjugations between elements of the constructs as shown and described. The present disclosure includes self-amplifying mRNAs that reduce the transcription numbers of sa-mRNA (e.g., nsP3) and subgenome (e.g., eGFP) to make less-cytopathic versions of the sa-mRNA. The present disclosure includes methods and constructs including structure-based engineering to control replication rate and interferon responses of sa-mRNAs.

In one aspect, the present disclosure provides constructs of the oncolytic viruses and pseudoviral variations thereof as shown and described, and methods of making and using the constructs. The present disclosure includes pseudoviral variations of oncolytic viruses that are replication-defective or replication-impaired.

The present disclosure includes a composition comprising a payload delivery system, and nucleic acid constructs encoding at least an oncolytic virus and sa-mRNA encoding one or more GOI for use for the treatment of proliferative diseases such as cancer.

The present invention relates to a method for treating a disease or disorder comprising administering a composition comprising a payload delivery system, one or more nucleic acid construct(s) encoding at least one structural protein an oncolytic virus and one or more GOI encoded by an sa-mRNA. In particular, the composition as described herein is for use for treating a proliferative disease and, especially, for treating a cancer in a subject having or at risk of having a cancer.

Although the present disclosure is described in detail below, it is to be understood that this disclosure is not limited to the particular methodologies, protocols and reagents described herein as these may vary. It is also to be understood that the terminology used herein is for the purpose of describing particular aspects only, and is not intended to limit the scope of the present disclosure. Unless defined otherwise, all technical and scientific terms used herein have the same meanings as commonly understood by one of ordinary skill in the art.

Additionally, several documents are cited throughout the text of this specification. Each of the documents cited herein (including all patents, patent applications, scientific publications, manufacturer's specifications, instructions, etc.), whether supra or infra, are hereby incorporated by reference in their entirety.

### Definitions

As used herein, the terms “approximately” and “about,” as applied to one or more values of interest, refer to a value that is +10% of the recited value.

As used herein, the terms “obtained from”, “derived from”, “originating” and “originate” are used to identify the original source of a component (e.g. polypeptide, nucleic acid molecule) but is not meant to limit the method by which the component is made which can be, for example, by chemical synthesis or recombinant means.

As used herein, the term “oncolytic virus” refers to a virus capable of selectively replicating in dividing cells (e.g. a proliferative cell such as a cancer cell) with the aim of slowing the growth and/or lysing said dividing cell, either in vitro or in vivo, while showing no or minimal replication in non-dividing cells. Typically, an oncolytic virus contains a viral genome packaged into a viral particle (or virion) and is infectious (i.e. capable of infecting and entering into a host cell or subject). A oncolytic virus may be a pseudovirus.

As used herein, the term “pseudoviral particle”, “pseudovirus particle” or “pseudovirus” refers to a recombinant virus which lack certain gene sequences of the wild-type (WT) virus(es) from which it was derived. For example, a pseudovirus may be composed of core or backbone and surface proteins derived from different viruses and/or are able to infect susceptible host cells but can only replicate intracellularly for a single round. For example, a pseudoviral particle may be composed of at least one virus structural protein derived from the same or different wildtype viruses.

As used herein, the term “pseudo-viral particle sequence”, “defective viral genome”, or “DVG” means that the nucleic acid sequence/viral genome contains all the genes necessary for the expression of a pseudo-retroviral particle with the exception of the envelope (env) or capsid (cap) gene and possibly comprising one or more mutations to attenuate infectiveness of the pseudo-retroviral system, modify the tropism of the pseudo-retroviral system compared to the virus from which it was derived, or deliver therapeutic genes.

As used herein the term “virus structural protein” or “structural protein” refer to a viral capsid protein, a viral envelope protein, a fragment thereof or a complex thereof. Thus, virus structural proteins used for the present invention may consist of or comprise a capsid protein and/or an envelope protein and/or a fragment thereof. Some viruses are naturally enveloped. Such viruses include, but are not limited to, the Retroviridae (e.g. HIV, Moloney Murine Leukaemia Virus, Feline Leukaemia Virus, Rous Sarcoma Virus), the Coronaviridae, the Herpesviridae, the Hepadnaviridae, and the Orthomyxoviridae (e.g. Influenza Virus). However, naturally non-enveloped viruses may form envelopes and these are also encompassed by the invention. Naturally non-enveloped viruses include the Picornaviridae, the Reoviridae, the Adenoviridae, the Papillomaviridae and the Parvoviridae.

As used herein the term “viral capsid”, “capsid” or “cap” refer to a viral capsid protein. As used herein the term “viral envelope”, “envelope” or “env” refer to a viral envelope protein.

As used herein, the terms “gene of interest,” “genes of interest,” “gene or genes of interest,” or “GOI” refers to the nucleotide sequence which encode the therapeutic polypeptide, prophylactic polypeptide, diagnostic polypeptide, antigen, non-coding gene that encodes regulatory structure, or reporter as a result of expression of the payload, such as a nucleic acid molecule, including a mRNA or a sa-mRNA. For example, a GOI, for the purposes of this disclosure, includes, but is not limited to, polynucleotides encoding immunomodulators (such as IL12 and IL21).

As used herein, the term “modified nucleotide” refers to a nucleotide that contains one or more chemical modifications (e.g. substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. These modified nucleobases can be engineered to provide polynucleotide molecules having enhanced properties, e.g., increased stability such as resistance to nucleases. There are more than 96 naturally occurring modified nucleosides found on mammalian RNA. See, e.g., Limbach et al, Nucleic Acids Research, 22(12):2183-2196 (1994). Modified nucleosides may include a compound selected from the following non-limiting group: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, N1-Methylpseudouridine-5′-Triphosphate, and N2,N2-dimethyl-6-thio-guanosine. The preparation of nucleotides and modified nucleotides and nucleosides are well-known in the art, e.g. from U.S. Pat. Nos. 4,373,071, 4,458,066, 4,500,707, 4,668,777, 4,973,679, 5,047,524, 5,132,418, 5,153,319, 5,262,530, 5,700,642 all of which are incorporated by reference in their entirety herein, and many modified nucleosides and modified nucleotides are commercially available.

As used herein, the term “regulatory element” refers to a nucleotide sequence that controls, at least in part, the transcription of a gene or genes of interest. Regulatory elements may include promoters, enhancers, and other nucleic acid sequences (e.g., polyadenylation signals) that control or help to control nucleic acid transcription or translation. Examples of transcription regulatory elements are described, for example, in Goeddel, Gene Expression Technology: Methods in Enzymology 185 (Academic Press, San Diego, Calif, 1990).

As used herein, the term “open reading frame”, “ORF”, “expression unit” or “coding region” refer to a sequence of nucleotides that is capable of transcribing or translating into one or more gene products. A gene product may comprise, but is not limited to, a polypeptide, a protein, a regulatory structure, or a combination thereof.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a GOI if the promoter modulates transcription of said GOI in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “linker” refers to a moiety connecting two moieties, for example, a nucleotide sequence added between two nucleotide sequences to connect said two nucleotide sequences. There is no particular limitation regarding the linker sequence.

As used herein, the term “subgenomic promoter”, is a promoter that can be used to transcribe the subgenome of alphaviruses encoding structural proteins by RNA dependent RNA polymerase encoded by nsP. When two or more subgenomic promoters are present in a nucleic acid comprising multiple expression units, the subgenomic promoters can be the same or different. In certain aspects, subgenomic promoters can be modified using techniques known in the art in order to increase or reduce viral transcription of the proteins, see e.g., U.S. Pat. No. 6,592,874, which is incorporated by reference in its entirety herein.

As used herein, “expression” of a nucleic acid sequence refers to transcription of DNA into RNA (such as a regulatory element or mRNA, including sa-mRNA) and/or translation of an mRNA into a peptide, polypeptide, or protein, assembly of multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalents, are used inter-changeably.

As used herein, the term “reporter” relates to a molecule, typically a peptide or protein, which is encoded by a reporter gene and measured in a reporter assay. Existing systems usually employ an enzymatic reporter (e.g. GFP or Luciferase) and measure the activity of said reporter.

As used herein, the phrase “biologically active” refers to a characteristic of any substance that has activity in a biological system and/or organism. The term “biologically active agent” refers to any agent that, when administered to a subject, has a therapeutic, diagnostic, and/or prophylactic effect and/or elicits a desired biological and/or pharmacological effect. Such agents include, but are not limited to, cytotoxins, radioactive ions, chemotherapeutic agents, small molecule drugs, proteins, and nucleic acids, such as therapeutic RNA. As used herein, the term “therapeutic RNA” refers to a RNA molecule that has activity and/or elicits a desired biological and/or pharmacological effect in a biological system and/or organism.

As used herein, the term “payload” refers to a moiety whose biological activity is desired to be delivered (in)to and/or localize at a cell or tissue. Payloads include, but are not limited to biologically active agents and the like. In some aspects, the payload may be a nucleic acid that encodes a protein or polypeptide. In some aspects, the payload may include or encode a cytokine, a chemokine, an antibody or antibody fragment, a receptor or receptor fragment, an enzyme, an enzyme inhibitor, a hormone, a lymphokine, a plasminogen activator, a natural or modified immunoglobulin or a fragment thereof, an antigen, a chimeric antibody receptor, variable or hypervariable regions of light and/or heavy chains of an antibody (V_{L}, V_{H}), variable fragments (Fv), Fab′ fragments, F(ab′) 2 fragments, Fab fragments, single chain antibodies (scAb), single chain variable regions (scFv), complementarity determining regions (CDR), domain antibodies (dAbs), single domain heavy chain immunoglobulins of the BHH or BNAR type, single domain light chain immunoglobulins, or other polypeptides known in the art containing an AB capable of binding target proteins or epitopes on target proteins, or any other desired biological macromolecule. In some aspects, the payload may include or encode a regulatory element. In some aspects, the payload may include or encode a small interfering RNA (siRNA), a micro-RNA (miRNA), an asymmetrical interfering RNA (aiRNA), a Dicer-substrate RNA (dsRNA), a small hairpin RNA (shRNA), small activating RNA (saRNA), transfer RNA (tRNA), or long intergenic non-coding (lincRNA).

As used herein, the term “transcription” comprises “in vivo transcription” and “in vitro transcription” wherein the term “in vitro transcription” relates to a method in which RNA, in particular sa-mRNA, is synthesized in vitro in a cell-free manner.

As used herein, two nucleic acids are substantially homologous when the nucleotide sequences have at least about 90%, 91%, 92%, 93%, 94%, 95%, 96%, 97%, 98%, or 99% identity. The percent identity between the two sequences is a function of the number of identical positions shared by the sequences, taking into account the number of gaps, and the length of each gap, which need to be introduced for optimal alignment of the two sequences. Sequence homology for nucleic acids, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997).

As used herein, “encapsulation efficiency” refers to the amount of a biological agent that becomes part of a nanoparticle composition, relative to the initial total amount of biologically active agent used in the preparation of a nanoparticle composition. For example, if 97 mg of biologically active agent are encapsulated in a nanoparticle composition out of a total 100 mg of biologically active agent initially provided to the composition, the encapsulation efficiency may be given as 97%. As used herein, “encapsulation” may refer to complete, substantial, or partial enclosure, confinement, surrounding, or encasement.

As used herein, a “payload delivery system” or “delivery system” are systems to deliver biologically active agents/payloads into target host cells. Such delivery systems include, for example lipid nanoparticle based delivery (Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Feigner et al (1987) Proc. Natl. Acad. Sci. USA 84: 7413-7414), as well as use of viral vectors {e.g., adenoviral (see, e.g., Berns et al (1995) Ann. NY Acad. Sci. 772: 95-104; AIi et al (1994) Gene Ther. 1: 367-384; and Haddada et al. (1995) Curr. Top. Microbiol. Immunol. 199 (Pt 3): 297-306 for review), papillomaviral, retroviral (see, e.g., Buchscher et al. (1992) J. Virol. 66(5) 2731-2739; Johann et al. (1992) J. Virol. 66 (5): 1635-1640 (1992); Sommerfelt et al, (1990) Virol. 176:58-59; Wilson et al. (1989) J. Virol. 63:2374-2378; Miller et al, J. Virol. 65:2220-2224 (1991); Wong-Staal et al, PCT/US94/05700, and Rosenburg and Fauci (1993) in Fundamental Immunology, Third Edition Paul (ed) Raven Press, Ltd., New York and the references therein, and Yu et al, Gene Therapy (1994) supra.), and adeno-associated viral vectors (see, West et al (1987) Virology 160:38-47; Carter et al (1989) U.S. Pat. No. 4,797,368; Carter et al WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al (1985) MoI. Cell. Biol. 5(11):3251-3260; Tratschin, et al (1984) MoI. Cell. Biol, 4:2072-2081; Hermonat and Muzyczka (1984) Proc. Natl. Acad. Sci. USA, 81:6466-6470; McLaughlin et al (1988) and Samulski et al (1989) J. Virol, 63:03822-3828), and the like.

The nucleic acid construct(s) encoding the pseudoviral particle sequence/defective viral genome (DVG) and the GOI may be carried together, either by the same plasmid or payload delivery system; or separately by separate nucleic acid molecules and payload delivery systems. Three particularly useful delivery systems are (i) LNPs (ii) non-toxic and biodegradable polymer microparticles (iii) cationic submicron oil-in-water emulsions. In one aspect, the payload of the present disclosure is delivered using LNPs.

As used herein, a “nanoparticle composition” or “LNP formulation” refer to a type of non-viral payload delivery system comprising one or more lipids. Nanoparticle compositions are typically sized on the order of micrometers or smaller and may include a lipid bilayer.

For example, the lipid component of a nanoparticle composition may include one or more cationic/ionizable, PEGylated, structural, or other lipids, such as phospholipids; or a LNP and lipid components of a nanoparticle composition according to the LNPs and lipid components disclosed in PCT Patent Application Nos. PCT/US2023/085919 and PCT/US2023/017777, which are fully incorporated herein by reference.

As used herein, a “lipid component” is that component of a nanoparticle composition that includes one or more lipids. For example, the lipid component may include one or more cationic/ionizable, PEGylated, structural, or other lipids, such as phospholipids.

As used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein the term “sterol” or “sterol derivative” refer to a class of compounds with a four ring 17 carbon cyclic structure which can further comprises one or more substitutions. A sterol derivative comprises any molecule having the 4-member ring structure characteristic of steroids and a hydroxyl (—OH) or ester (—OR) substitution at the 3-carbon position, for example, having the exemplary structure below:

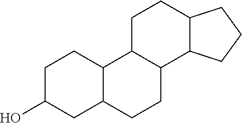 [SMILES: OC1CCC2C(CCC3C4CCCC4CCC23)C1]

The skilled artisan will understand that a sterol derivative can be further substituted at one or more of the other ring carbons, for example, with one or more non-alkyl substitutions, including alkyl groups, alkoxy groups, hydroxy groups, oxo groups, acyl groups, or a double bond between two or more carbon atoms:

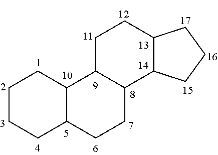 [SMILES: C1CCC2C(C1)CCC1C3CCCC3CCC21]

Sterols useful in the compositions and methods of the present disclosure may be selected from the following non-limiting group: cholesterol, fecosterol, sitosterol, ergosterol, campesterol, stigmasterol, brassicasterol, tomatidine, tomatine, ursolic acid, alpha-tocopherol, cholesteryl chloroformate, sitosterol, ergosterol, lanosterol, desmosterol, or a derivative thereof.

As used herein, the terms “PEG lipid” or “PEGylated lipid” refer to a lipid comprising a polyethylene glycol component. For example, a PEG lipid may be selected from the following non-limiting group: PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMIPE, PEG-DPPC, or a PEG-DSPE lipid.

As used herein, the terms “phospholipid” or “helper lipid” refer to a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell. In general, phospholipids may include a phospholipid moiety and one or more fatty acid moieties.

Phospholipids useful in the compositions and methods of the disclosure may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some aspects, a nanoparticle composition includes DSPC. In certain aspects, a nanoparticle composition includes DOPE. In some aspects, a nanoparticle composition includes both DSPC and DOPE.

As used herein, the term “substantially” refers to the qualitative condition of exhibiting total or near-total extent or degree of a characteristic or property of interest.

As used herein, “size” or “mean size” in the context of nanoparticle composition, such as an LNP, refers to the mean diameter of the LNP.

The present disclosure may include uses or methods of administration that may include intravenous, intranasal, intratracheal, intracerebral, intratumoral, intraperitoneal, intramuscular, intradermal, intravitreal, subretinal, subcutaneous, or other methods of delivering a composition to a subject. A method of administration may be selected to target delivery (e.g., to specifically deliver) to a specific region, cell, tumor, organ, or system of a body.

As used herein, the terms “analog,” “mutant”, “modified” and “variant” refer to a molecule (polypeptide or nucleic acid) exhibiting one or more sequence modification(s) with respect to the native or wildtype counterpart. Any modification(s) can be envisaged, including substitution, insertion and/or deletion of one or more nucleotide/amino acid residue(s). Preferred are analogs that retain a degree of sequence identity of at least 80%, preferably at least 85%, more preferably at least 90%, and even more preferably at least 98% identity with the sequence of the native counterpart.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. In the context of the invention, the term “host cells” include prokaryotic cells, lower eukaryotic cells such as yeast, and other eukaryotic cells such as insect cells, plant and mammalian (e.g. human or non-human) cells as well as cells capable of producing the oncolytic virus and/or therapeutic RNA(s) for use in the invention. This term also includes cells which can be or has been the recipient of the biologically active agents described herein as well as progeny of such cells.

The expression “target cell” means the cell which it is desired to treat by introduction of a biologically active agent.

As used herein, the term “proliferative disease” encompasses any disease or condition resulting from uncontrolled cell growth and spread including cancers as well as diseases associated to an increased osteoclast activity (e.g. rheumatoid arthritis, osteoporosis, etc.) and cardiovascular diseases (restenosis that results from the proliferation of the smooth muscle cells of the blood vessel wall, etc.). The term “cancer” may be used interchangeably with any of the terms “tumor”, “malignancy”, “neoplasm”, etc. These terms are meant to include any type of tissue, organ or cell, any stage of malignancy (e.g. from a prelesion to stage IV).

### Oncolytic Virus

The oncolytic virus for use in the present invention can be obtained from any member of virus identified at present time provided that it is oncolytic by its propensity to selectivity replicate and kill dividing cells as compared to non-dividing cells. It may be a native virus that is naturally oncolytic or may be engineered by modifying one or more viral genes so as to increase tumor selectivity and/or preferential replication in dividing cells, such as those involved in DNA replication, nucleic acid metabolism, host tropism, surface attachment, virulence, lysis and spread (see for example Kirn et al., 2001, Nat. Med. 7: 781; Wong et al., 2010, Viruses 2: 78-106). One may also envisage placing one or more viral gene(s) under the control of event or tissue-specific regulatory elements (e.g. promoter).

Exemplary oncolytic viruses include, without limitation, Eastern Equine Encephalitis virus (EEE), Venezuelan Equine Encephalitis virus (VEE), Everglades virus (EVEV), Mucambo virus (MUCV), Pixuna virus (PIXV), Western Equine Encephalitis virus (WEE), Sindbis virus (SINV), Semliki Forest virus (SFV), Middelburg virus (MIDV), Chikungunya virus (CHIKV), O'nyong-nyong virus (ONNV), Ross River virus (RRV), Barmah Forest virus (BFV), Getah virus (GETV), Sagiyama virus (SAG), Bebaru virus (BEBV), Mayaro virus (MAYV), Una virus (UNAV), Aura virus (AURAV), Whataroa virus (WHAV), Babanki virus (BBKV), Kyzylagach virus (KYZV), Highlands J virus (HJV), Fort Morgan virus (FMV), Ndumu virus (NDUV), Buggy Creek virus (BCRV), reovirus, Seneca Valley virus (SVV), vesicular stomatitis virus (VSV), Newcastle disease virus (NDV), herpes simplex virus (HSV), morbillivirus virus, retrovirus, influenza virus, Sinbis virus, poxvirus, adenovirus, or the like.

In one aspect, the at least one virus structural protein encoded by the second nucleic acid construct is from an alphavirus, adenovirus, a HSV, an echovirus, a polio virus, a vaccinia virus, a measles virus, a vesicular stomatitis, an orthomyxovirus, a parvovirus, a maraba virus, a coxsackievirus, an autonomous parvovirus, a myxoma virus, a Newcastle disease virus, a reovirus, a seneca valley virus morbillivirus virus, a retrovirus, an influenza virus, a sindbis virus, semliki forest virus, venezuelan equine encephalitis virus, or a poxvirus. In some aspects, the at least one virus structural protein is a H101 protein, a T-VEC protein, a ECHO-7 protein, a teserpaturev protein, a nadofaragene firadenovec protein or a HSV1 protein. In some aspects, the at least one virus structural protein is SEQ ID NO: 26, SEQ ID NO: 56, or SEQ ID NO: 57.

In one aspect, the oncolytic virus for use in the present invention is obtained from a VEE. Representative examples for use in the invention are described in the literature (e.g. Lundstrom, 2022, Front. Mol. Biosci. 9:864781).

In one aspect, the oncolytic virus for use in the present invention is a generated chimeric virus that is derived from Venezuelan equine encephalitis virus (VEE), the same technical principles as discussed herein may be applied to construct chimeric viruses derived from other viruses, such as alphaviruses (including western equine encephalitis virus, eastern equine encephalitis virus or other related viruses). The second nucleic acid construct encoding an mRNA encode structural proteins of VEE, which may include viral envelope glycoproteins, viral capsid proteins or both. A representative example of the strains of VEE from where the nucleic acid sequence is derived from may include but is not limited to the TC-83 strain.

### Non-Viral Payload Delivery System

In some aspects, the non-viral payload delivery system of the composition of the disclosure is a lipidoid, a polymer, a core-shell nanoparticle, a nanoparticle mimic, a lipid nanoparticle (LNP), a polymeric nanoparticle, a micelle, liposome, a virus-like particle (VLP), a lipoplex, a polyplex or a lipopolyplex. In some aspects, the non-viral payload delivery system is a LNP non-viral payload delivery system. In some aspects, the LNP comprises an ionizable lipid, a phospholipid, a steroid, a polyethylene glycol (PEG) lipid, a modular lipid, or a combination thereof.

In some aspects, the diameter of the LNP is 1 m or shorter (e.g., 1 m, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some aspects, nanoparticle compositions, such as LNPs, are vesicles including one or more lipid bilayers. In certain aspects, a nanoparticle composition includes two or more concentric bilayers separated by aqueous compartments. Lipid bilayers may be functionalized and/or crosslinked to one another. Lipid bilayers may optionally further include one or more ligands, proteins, or channels.

Typically the LNPs used as the delivery system in the research and development of new drugs, including FDA approved mRNA vaccines such as the mRNA COVID vaccines, and FDA approved siRNA therapies, such as the siRNA therapy for the treatment of polyneuropathy in people with hereditary transthyretin-mediated amyloidosis, use a four component LNP delivery system. In a four component LNP delivery system, phospholipids function to increase transfection efficacy of nucleic acids; cationic/ionizable lipids function to stabilize nucleic acids within the lipid nanoparticle; stabilizing lipids serve as the “lipid raft,” which stabilizes the integrity of the LNP; and PEG-lipids inhibit aggregation and prevent clearance by macrophages, monocytes, or other phagocytic cells in vivo. The LNPs of the present disclosure may be a two-, three-, four-, five- or more component LNP.

### Cationic/Ionizable Lipids

As used herein, the terms “ionizable lipid” or “cationic lipid” are lipids that may have a positive or partial positive charge at physiological pH. A nanoparticle composition may include one or more ionizable lipids in addition to a lipid according to lipids disclosed in PCT Patent Application No. PCT/US2023/017777, which is fully incorporated herein. A nanoparticle composition may include one or more ionizable lipids in addition to a lipid according to Formula (I)-(VII).

In one aspect, the present disclosure provides an LNP comprising an ionizable lipid compound selected from Formulae I, II, IV, V, IV, and VII, wherein Formula I is

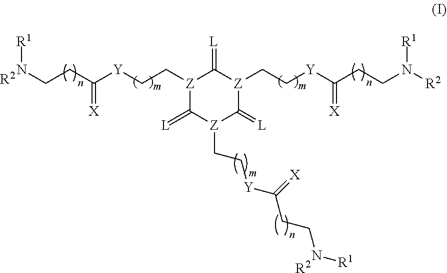

or a salt or isomer thereof; wherein Formula II is

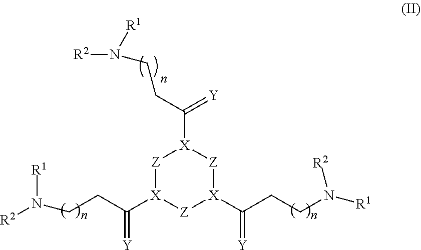

or a salt or isomer thereof; wherein Formula IV is

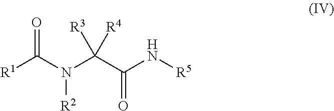

or a salt or isomer thereof; wherein Formula V is

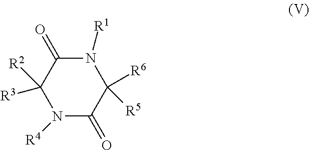

or a salt or isomer thereof; wherein Formula VI is

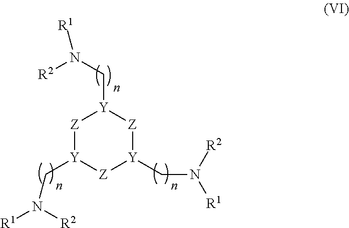

or a salt or isomer thereof; wherein Formula VII is

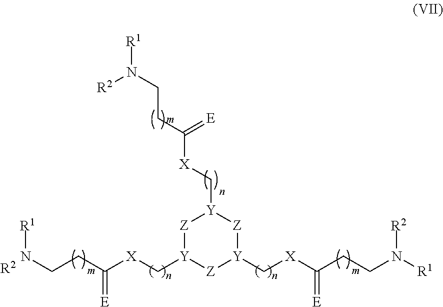

or a salt or isomer thereof; wherein, each m and n are independently an integer from 0-10;
each R¹, R², R³, R⁴, R⁵ and R⁶ are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl,

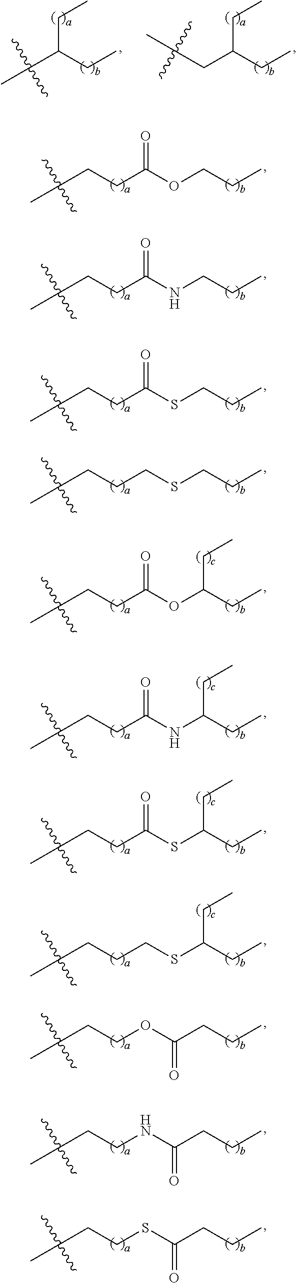

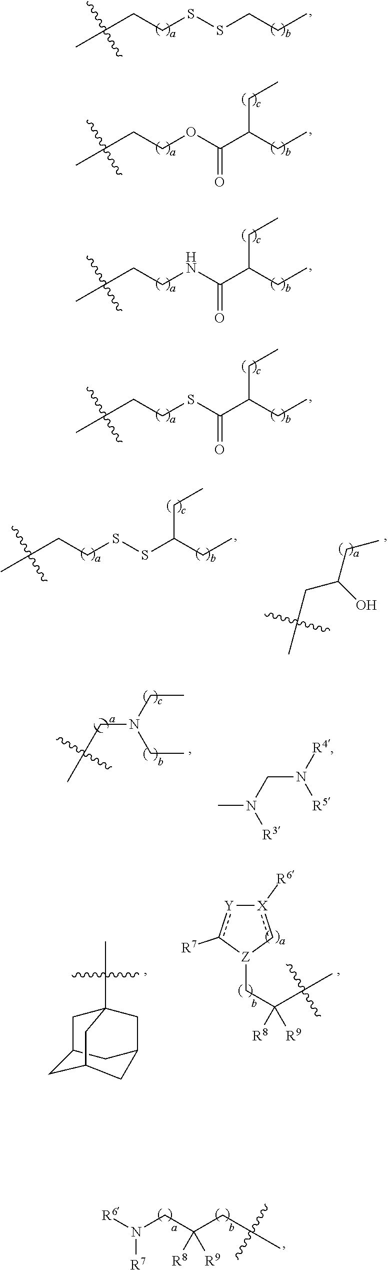

a, b and c are each independently an integer from 0-24;
each R^{6′}, R⁷, R⁸ and R⁹ are independently selected from H, C1-C24 alkyl, C1-C24 alkenyl, C1-C24 alkynyl, substituted alkyl, substituted alkenyl, substituted alkynyl, substituted acyl,

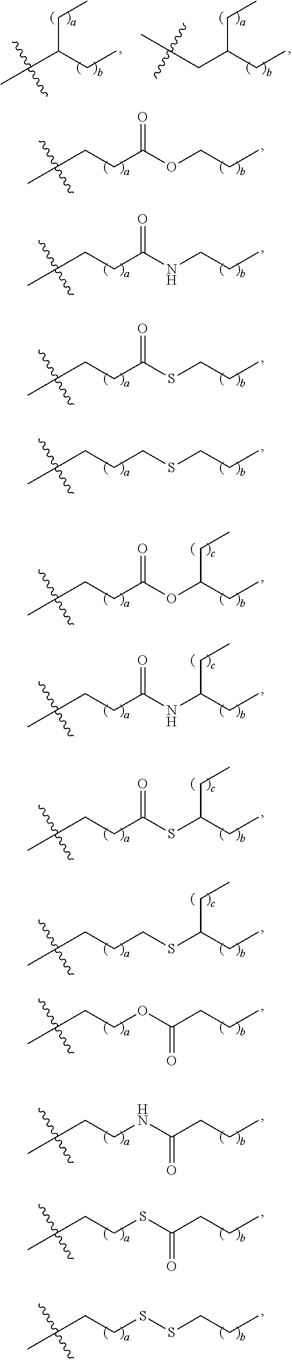

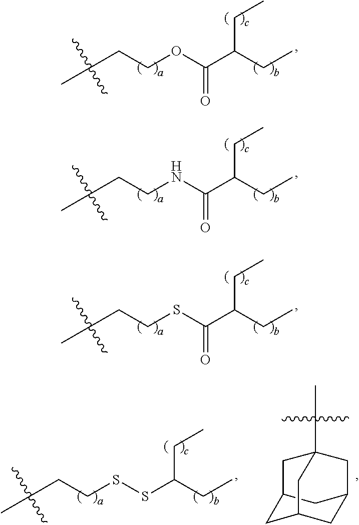

each R^{3′}, R^{4′} and R^{5′} are independently selected from H, C1-C24 alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl,

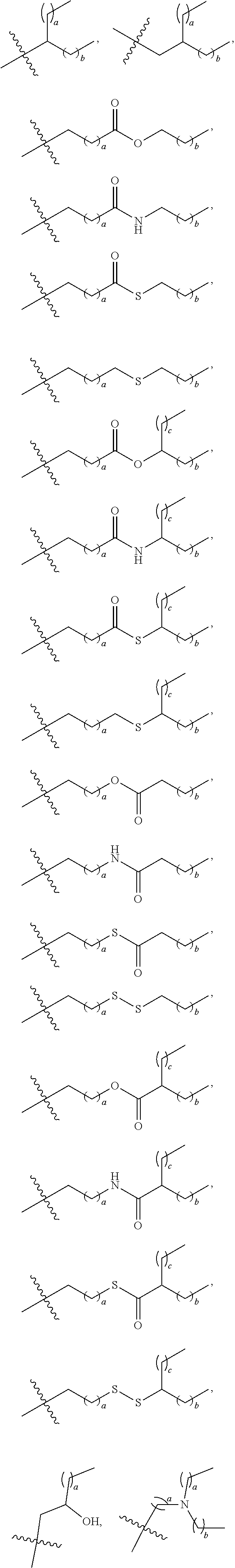

a, b and c ae each independently an integer from 0-24;
each X is independently selected from CH₂, NH, O, or S;
each Y is independently selected from CH₂, NH, O, or S;
each Z is independently selected from CH, N, O, or S; and
each E is independently selected from CH₂, NH, O, or S.

In some aspects, the present disclosure provides an LNP comprising an ionizable lipid compound selected from Formula III, wherein Formula III is

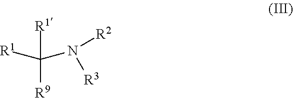

or a salt or isomer thereof, wherein
each n is independently an integer from 0-10;
each R¹, R^{1′}, R² and R³ are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl,

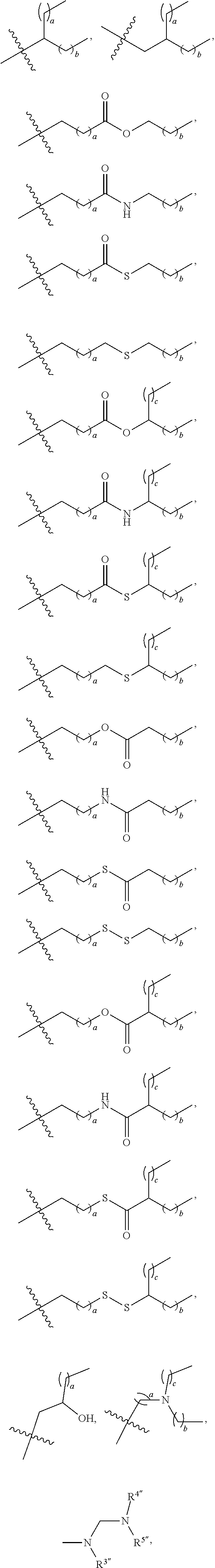

a, b and c are each independently an integer from 0-24;
each R⁹ is independently selected from

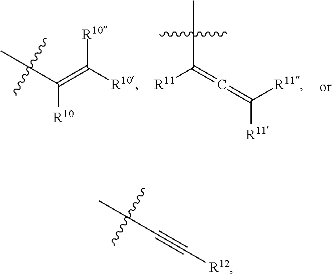

wherein each R¹⁰, R^{10′}, R^{10″}, R¹¹, R^{11′}, R^{11″}, and R¹² are independently selected from H, C1-C24 alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl,

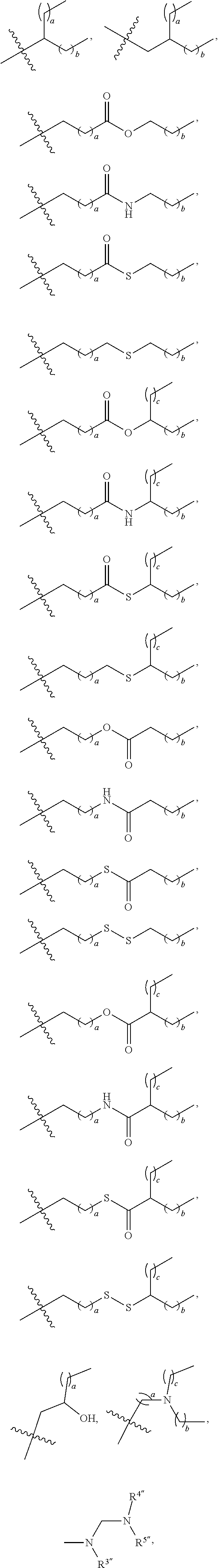

a, b and c are each independently an integer from 0-24;
R^{3″}, R^{4″}, and R^{5″} are independently selected from H, C1-C24 alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl,

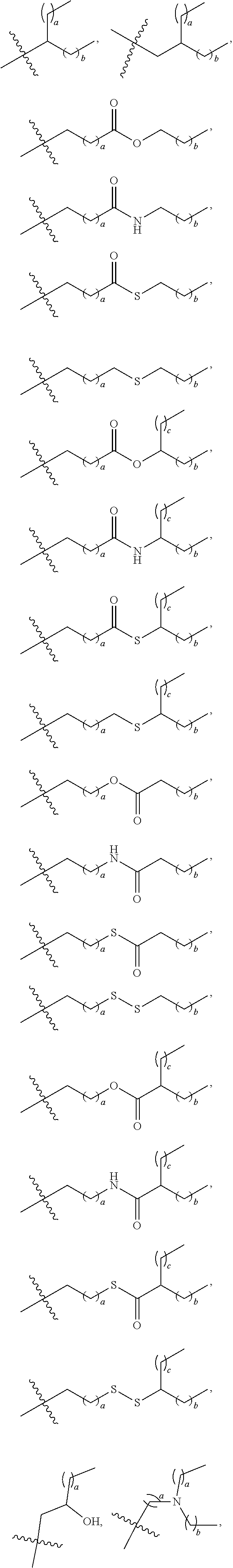

a, b and c are each independently an integer from 0-24

In some aspects, the LNP comprises an ionizable lipid compound, wherein the ionizable lipid is.

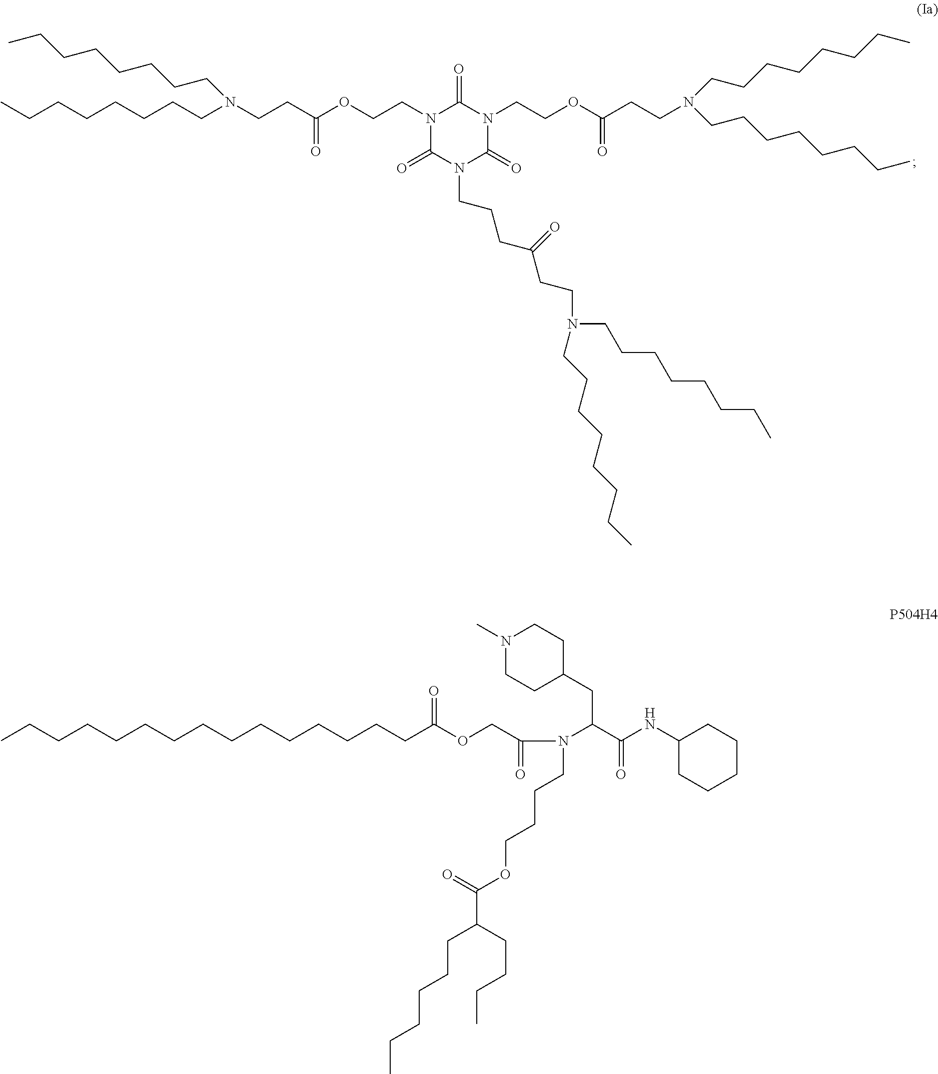

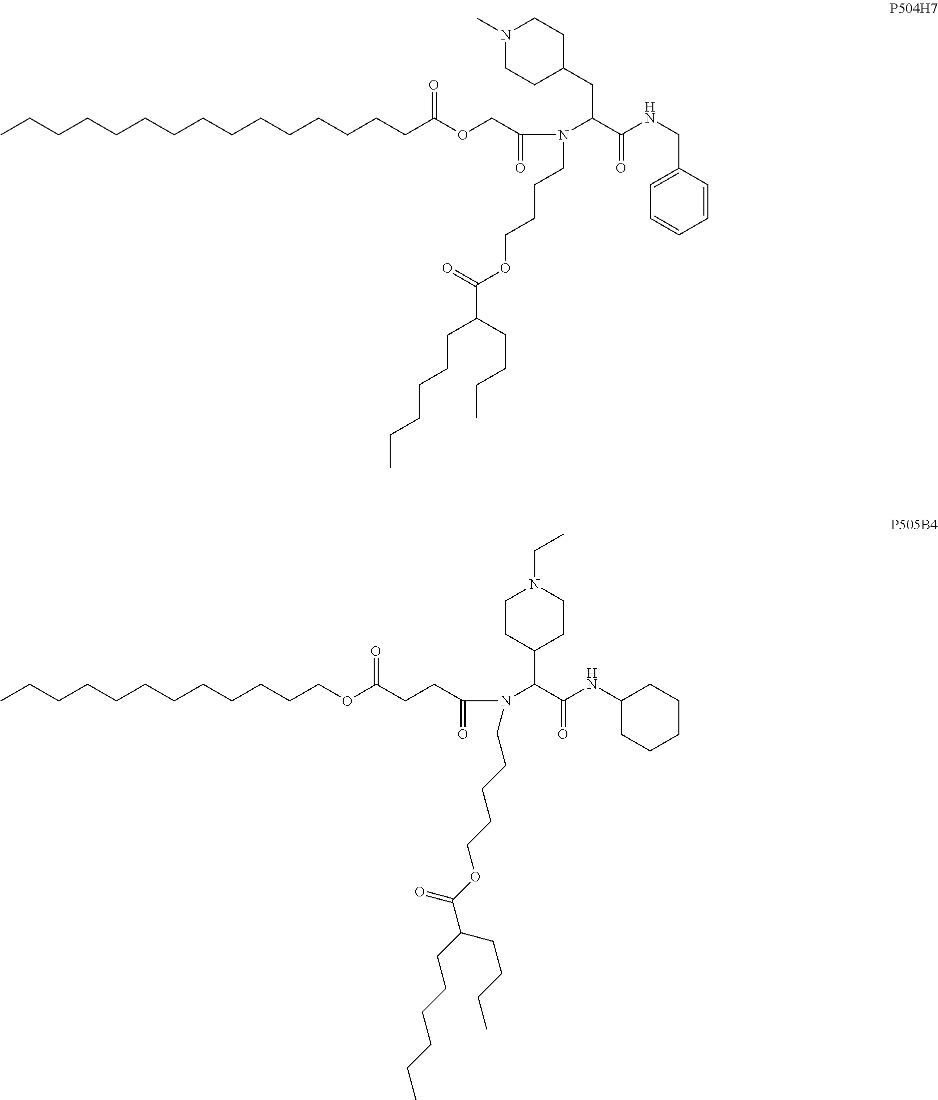 [SMILES: CCCCCCCCCCCCCCCC(=O)OCC(=O)N(CCCCOC(=O)C(CCCC)CCCCCC)C(CC1CCN(C)CC1)C(=O)NCc1ccccc1 ; CCCCCCCCCCCCOC(=O)CCC(=O)N(CCCCCOC(=O)C(CCCC)CCCCCC)C(C(=O)NC1CCCCC1)C1CCN(CC)CC1]

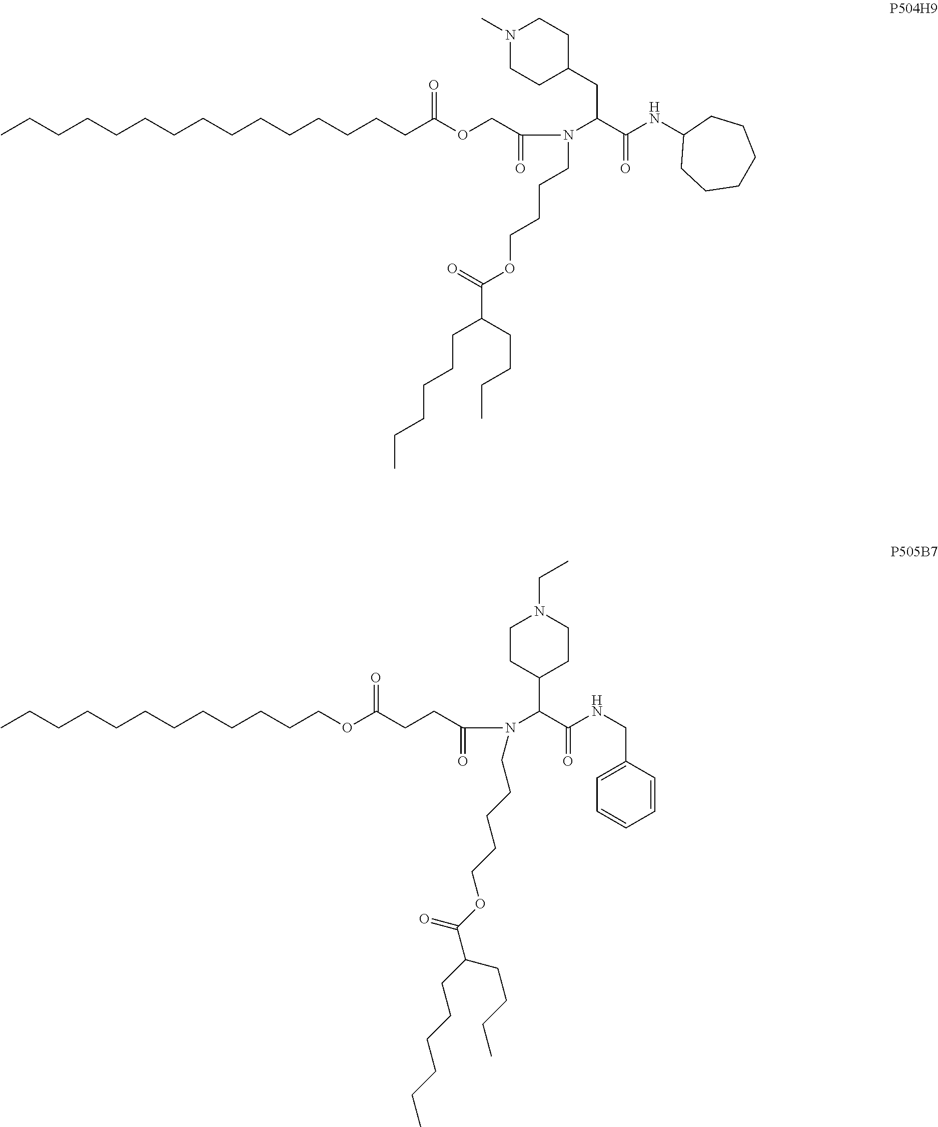 [SMILES: CCCCCCCCCCCCCCCC(=O)OCC(=O)N(CCCCOC(=O)C(CCCC)CCCCCC)C(CC1CCN(C)CC1)C(=O)NC1CCCCCC1 ; CCCCCCCCCCCCOC(=O)CCC(=O)N(CCCCCOC(=O)C(CCCC)CCCCCC)C(C(=O)NCc1ccccc1)C1CCN(CC)CC1]

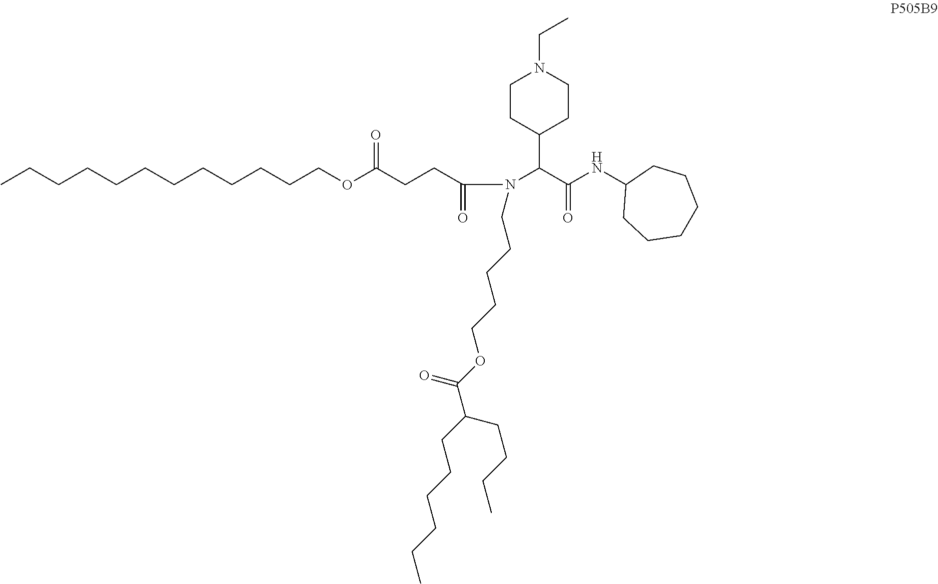 [SMILES: CCCCCCCCCCCCOC(=O)CCC(=O)N(CCCCCOC(=O)C(CCCC)CCCCCC)C(C(=O)NC1CCCCCC1)C1CCN(CC)CC1]

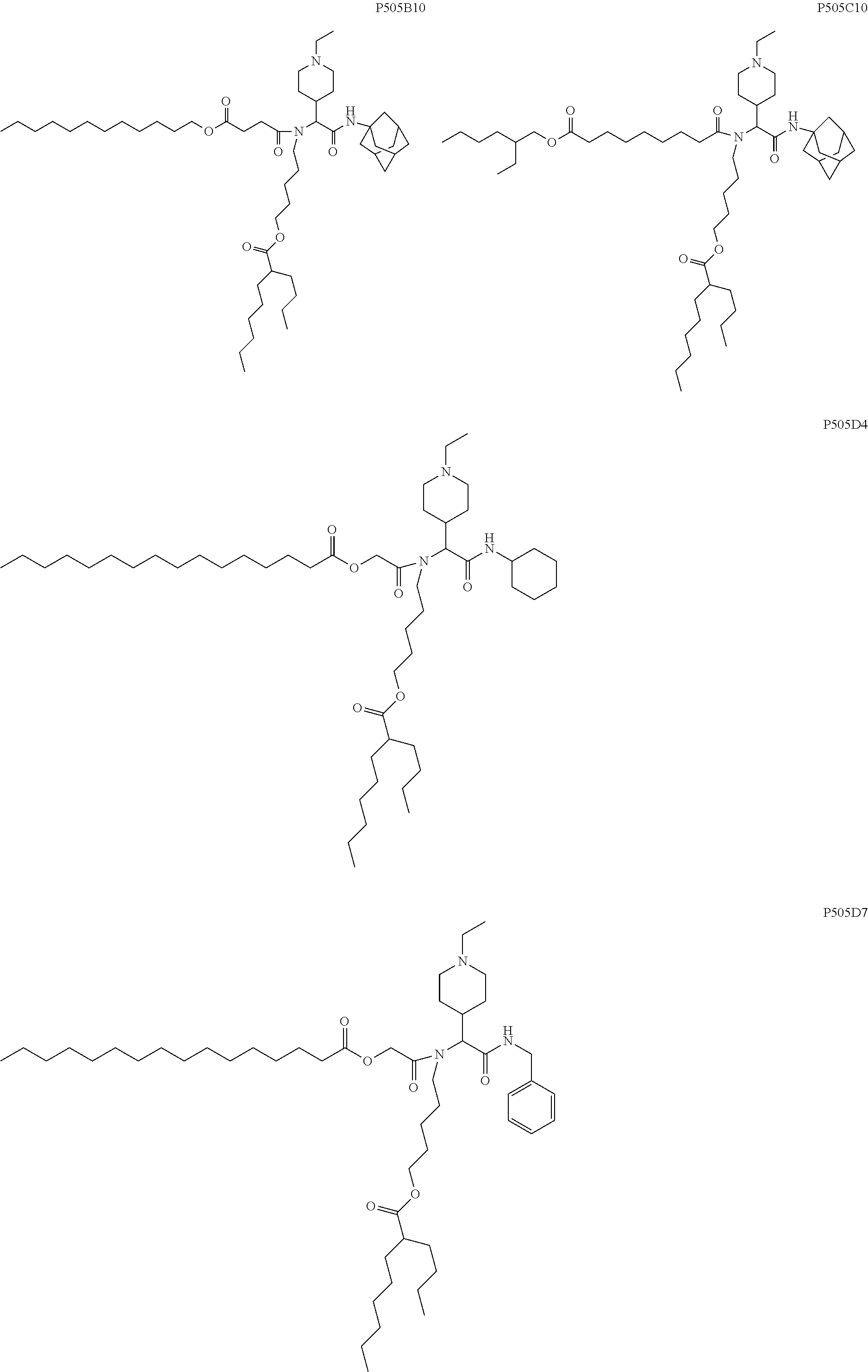 [SMILES: CCCCCCCCCCCCOC(=O)CCC(=O)N(CCCCCOC(=O)C(CCCC)CCCCCC)C(C(=O)NC12CC3CC(CC(C3)C1)C2)C1CCN(CC)CC1 ; CCCCCCC(CCCC)C(=O)OCCCCCN(C(=O)CCCCCCCC(=O)OCC(CC)CCCC)C(C(=O)NC12CC3CC(CC(C3)C1)C2)C1CCN(CC)CC1 ; CCCCCCCCCCCCCCCC(=O)OCC(=O)N(CCCCCOC(=O)C(CCCC)CCCCCC)C(C(=O)NC1CCCCC1)C1CCN(CC)CC1 ; CCCCCCCCCCCCCCCC(=O)OCC(=O)N(CCCCCOC(=O)C(CCCC)CCCCCC)C(C(=O)NCc1ccccc1)C1CCN(CC)CC1]

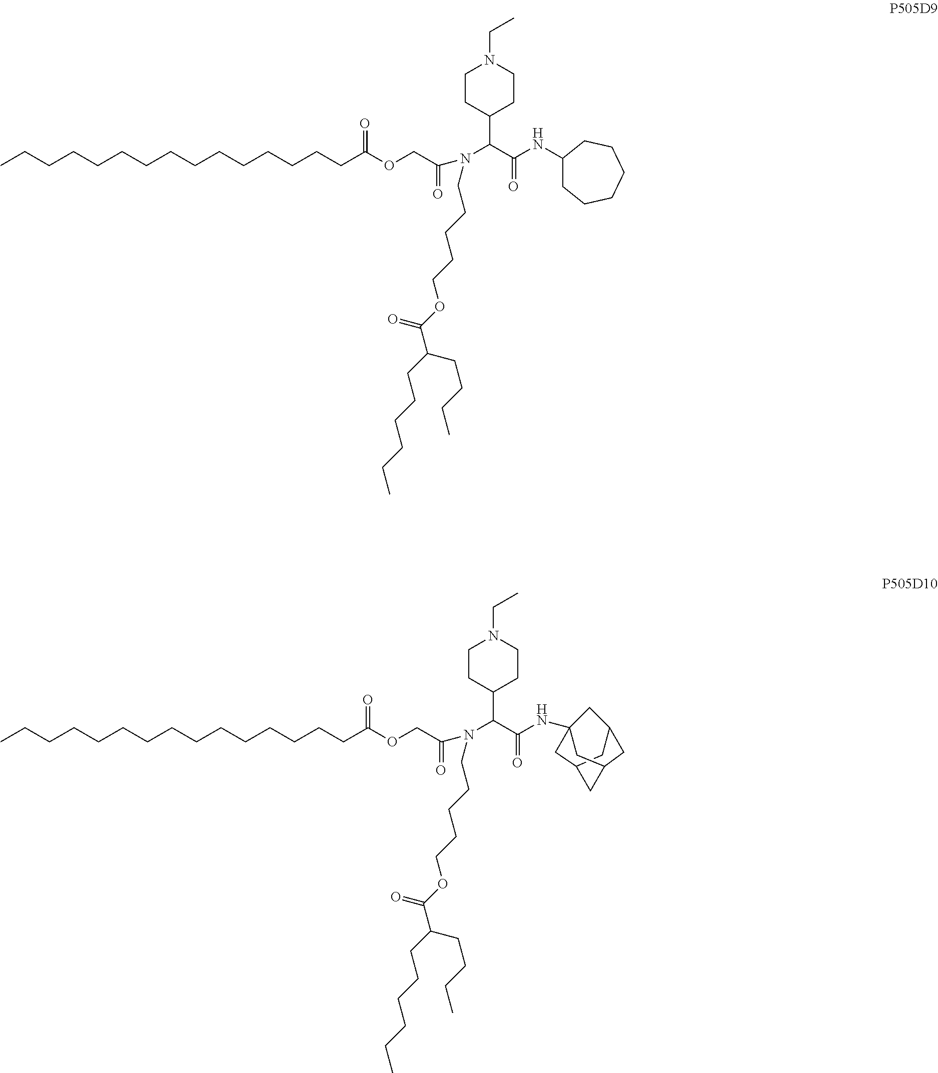 [SMILES: CCCCCCCCCCCCCCCC(=O)OCC(=O)N(CCCCCOC(=O)C(CCCC)CCCCCC)C(C(=O)NC1CCCCCC1)C1CCN(CC)CC1 ; CCCCCCCCCCCCCCCC(=O)OCC(=O)N(CCCCCOC(=O)C(CCCC)CCCCCC)C(C(=O)NC12CC3CC(CC(C3)C1)C2)C1CCN(CC)CC1]

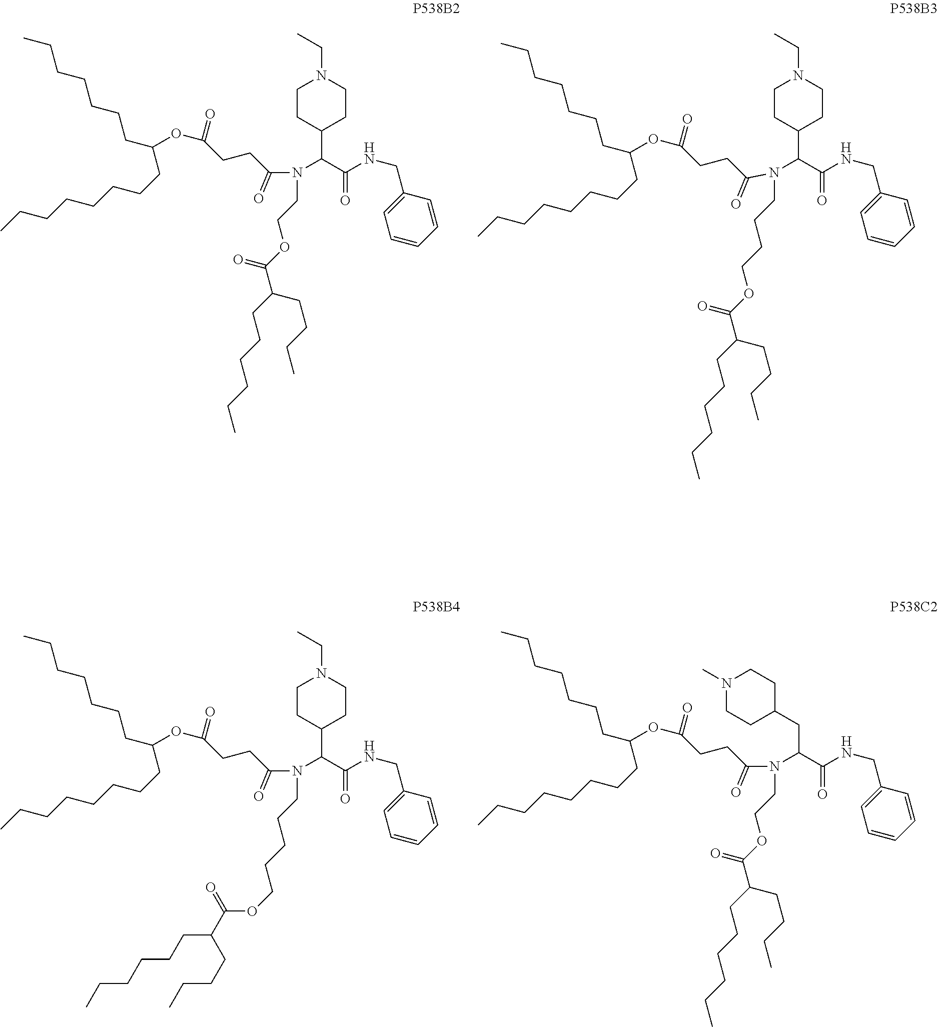 [SMILES: CCCCCCCCC(CCCCCCC)OC(=O)CCC(=O)N(CCOC(=O)C(CCCC)CCCCCC)C(C(=O)NCc1ccccc1)C1CCN(CC)CC1 ; CCCCCCCCC(CCCCCCC)OC(=O)CCC(=O)N(CCCCOC(=O)C(CCCC)CCCCCC)C(C(=O)NCc1ccccc1)C1CCN(CC)CC1 ; CCCCCCCCC(CCCCCCC)OC(=O)CCC(=O)N(CCCCCOC(=O)C(CCCC)CCCCCC)C(C(=O)NCc1ccccc1)C1CCN(CC)CC1 ; CCCCCCCCC(CCCCCCC)OC(=O)CCC(=O)N(CCOC(=O)C(CCCC)CCCCCC)C(CC1CCN(C)CC1)C(=O)NCc1ccccc1]

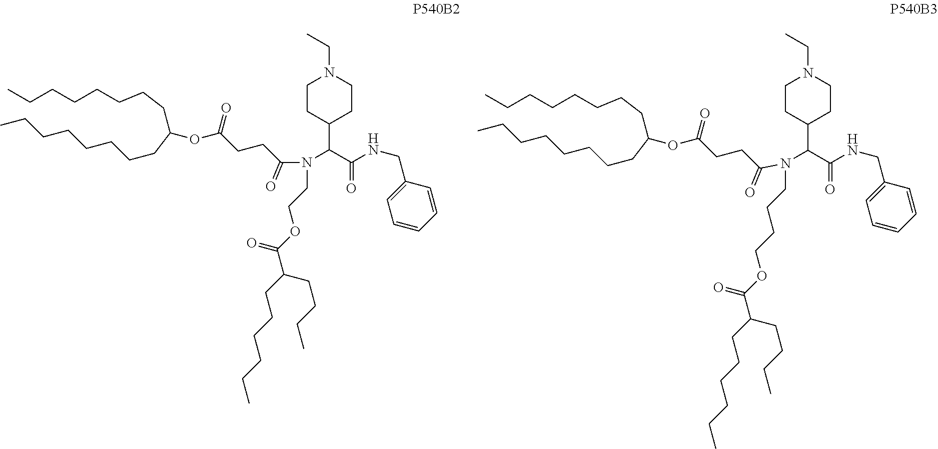 [SMILES: CCCCCCCCC(CCCCCCCC)OC(=O)CCC(=O)N(CCOC(=O)C(CCCC)CCCCCC)C(C(=O)NCc1ccccc1)C1CCN(CC)CC1 ; CCCCCCCCC(CCCCCCCC)OC(=O)CCC(=O)N(CCCCOC(=O)C(CCCC)CCCCCC)C(C(=O)NCc1ccccc1)C1CCN(CC)CC1]

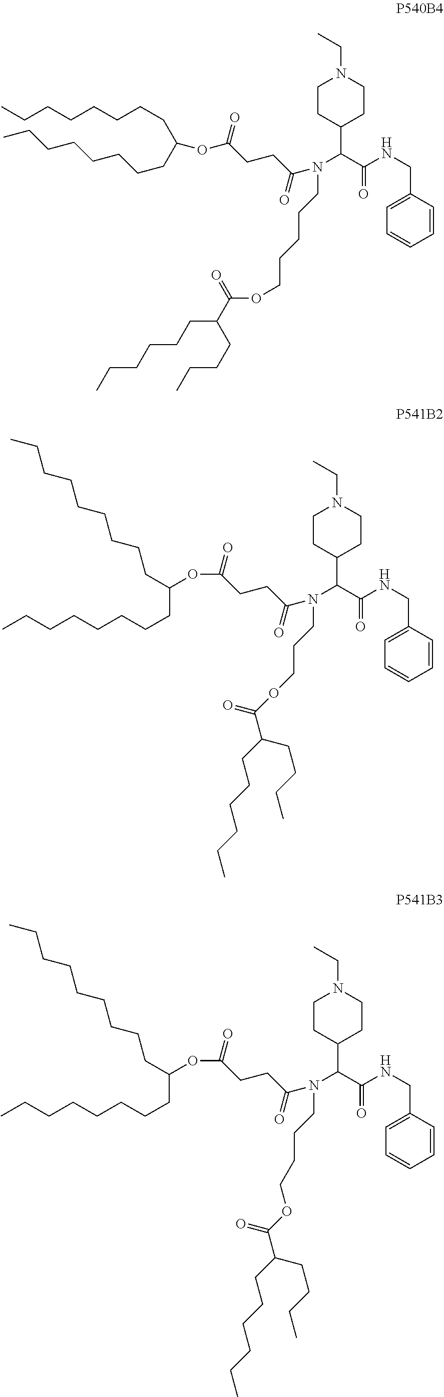 [SMILES: CCCCCCCCC(CCCCCCCC)OC(=O)CCC(=O)N(CCCCCOC(=O)C(CCCC)CCCCCC)C(C(=O)NCc1ccccc1)C1CCN(CC)CC1 ; CCCCCCCCCC(CCCCCCCC)OC(=O)CCC(=O)N(CCCOC(=O)C(CCCC)CCCCCC)C(C(=O)NCc1ccccc1)C1CCN(CC)CC1 ; CCCCCCCCCC(CCCCCCCC)OC(=O)CCC(=O)N(CCCCOC(=O)C(CCCC)CCCCCC)C(C(=O)NCc1ccccc1)C1CCN(CC)CC1]

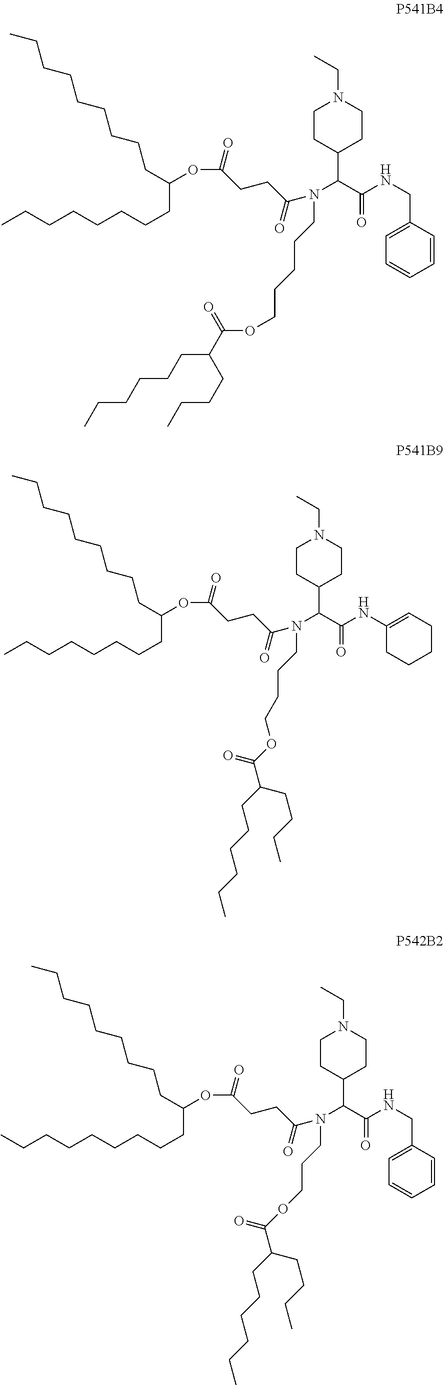 [SMILES: CCCCCCCCCC(CCCCCCCC)OC(=O)CCC(=O)N(CCCCCOC(=O)C(CCCC)CCCCCC)C(C(=O)NCc1ccccc1)C1CCN(CC)CC1 ; CCCCCCCCCC(CCCCCCCC)OC(=O)CCC(=O)N(CCCCOC(=O)C(CCCC)CCCCCC)C(C(=O)NC1=CCCCC1)C1CCN(CC)CC1 ; CCCCCCCCCC(CCCCCCCCC)OC(=O)CCC(=O)N(CCCOC(=O)C(CCCC)CCCCCC)C(C(=O)NCc1ccccc1)C1CCN(CC)CC1]

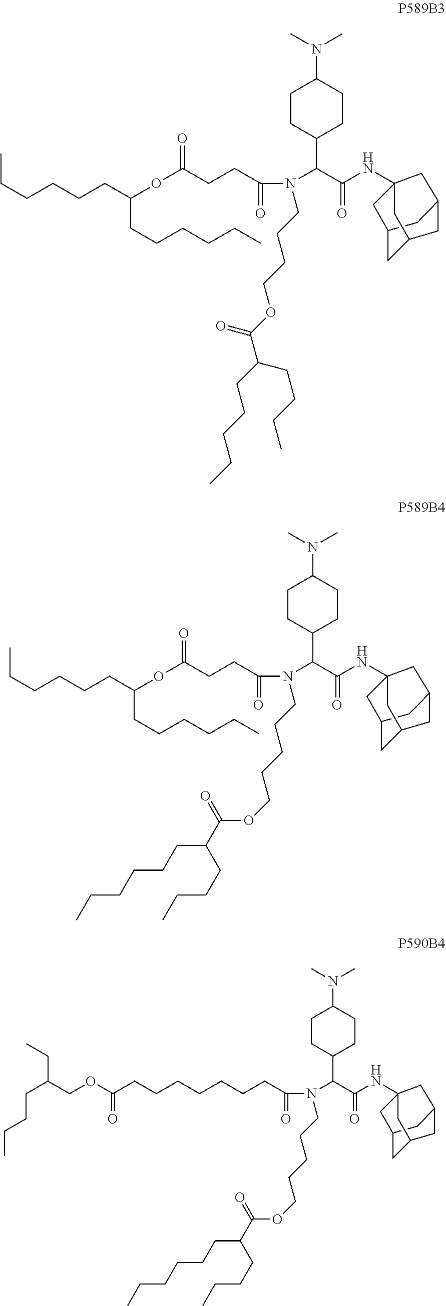 [SMILES: CCCCCCC(CCCCCC)OC(=O)CCC(=O)N(CCCCOC(=O)C(CCCC)CCCCC)C(C(=O)NC12CC3CC(CC(C3)C1)C2)C1CCC(N(C)C)CC1 ; CCCCCCC(CCCCCC)OC(=O)CCC(=O)N(CCCCCOC(=O)C(CCCC)CCCCCC)C(C(=O)NC12CC3CC(CC(C3)C1)C2)C1CCC(N(C)C)CC1 ; CCCCCCC(CCCC)C(=O)OCCCCCN(C(=O)CCCCCCCC(=O)OCC(CC)CCCC)C(C(=O)NC12CC3CC(CC(C3)C1)C2)C1CCC(N(C)C)CC1]

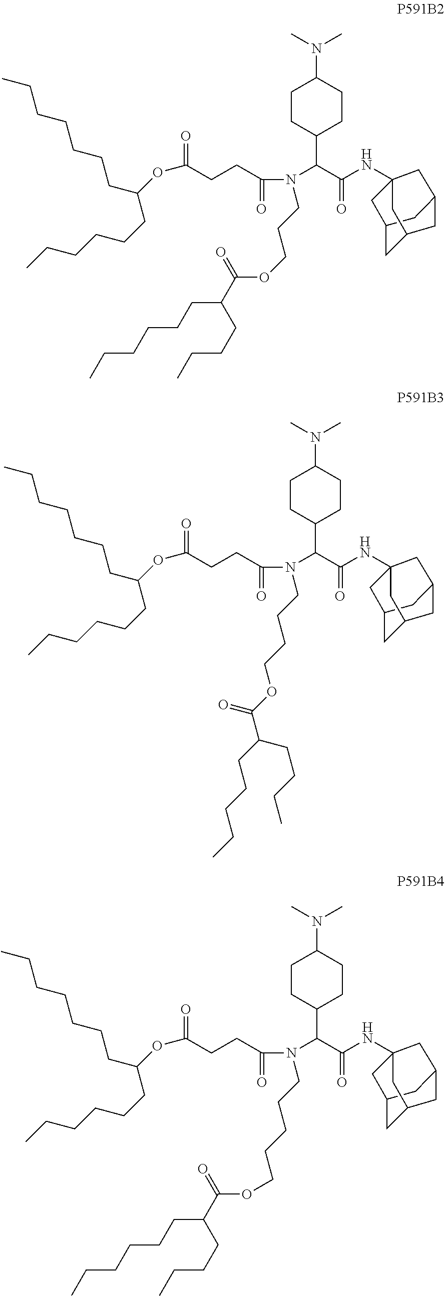 [SMILES: CCCCCCCC(CCCCCC)OC(=O)CCC(=O)N(CCCOC(=O)C(CCCC)CCCCCC)C(C(=O)NC12CC3CC(CC(C3)C1)C2)C1CCC(N(C)C)CC1 ; CCCCCCCC(CCCCCC)OC(=O)CCC(=O)N(CCCCOC(=O)C(CCCC)CCCCC)C(C(=O)NC12CC3CC(CC(C3)C1)C2)C1CCC(N(C)C)CC1 ; CCCCCCCC(CCCCCC)OC(=O)CCC(=O)N(CCCCCOC(=O)C(CCCC)CCCCCC)C(C(=O)NC12CC3CC(CC(C3)C1)C2)C1CCC(N(C)C)CC1]

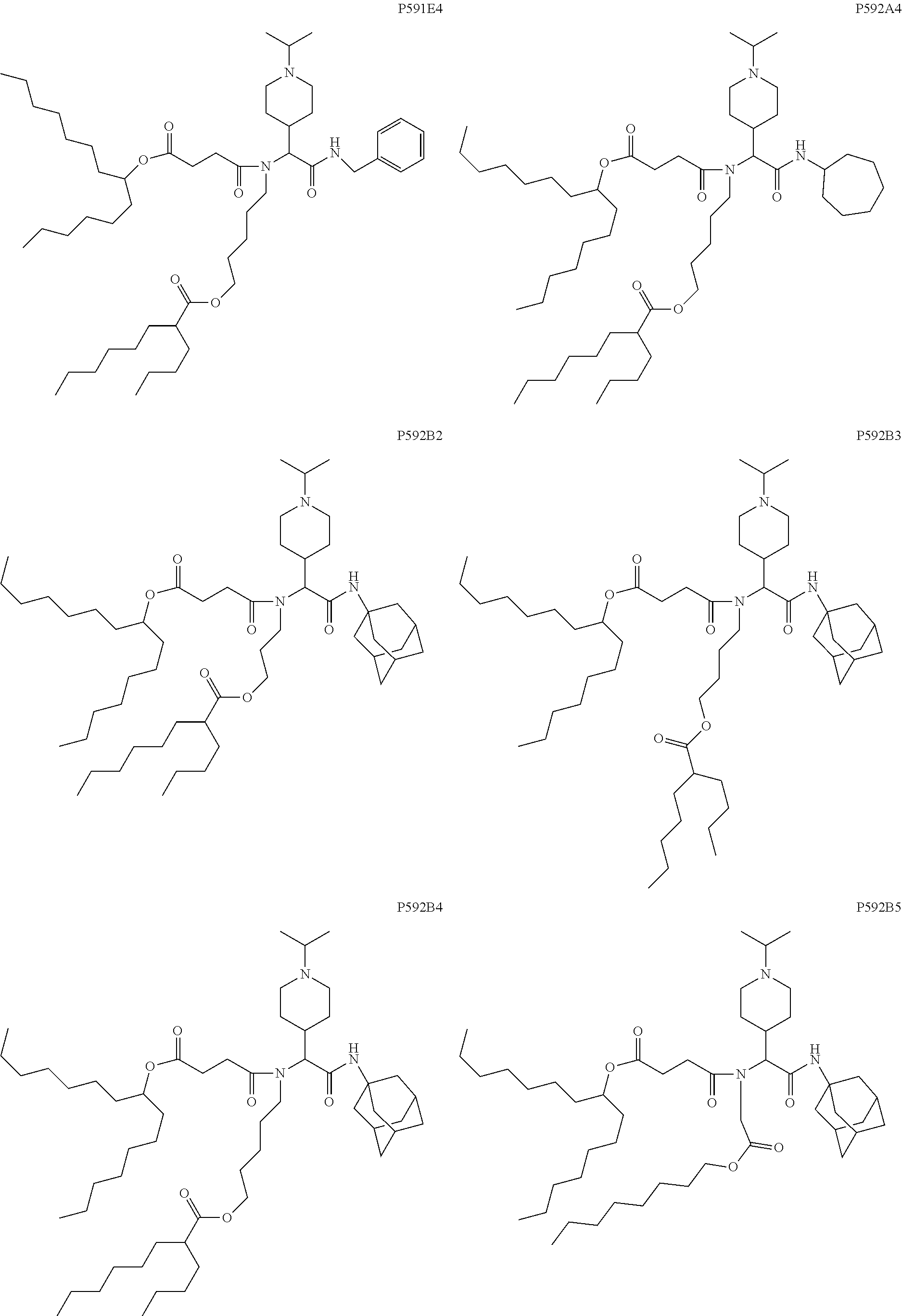

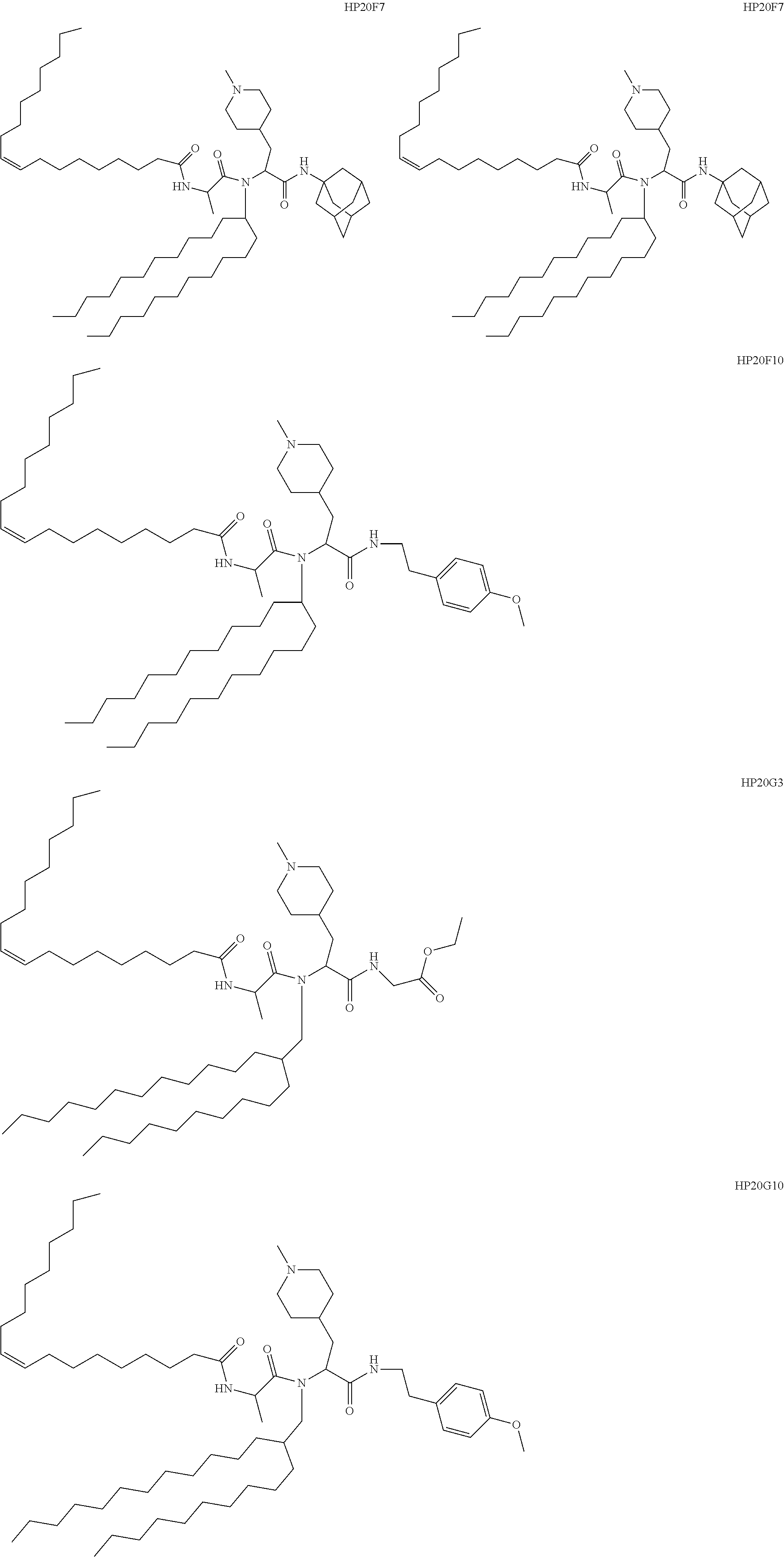

or
a salt or isomer thereof.

### Modular Lipids

As used herein, the term “modular lipid” refer to lipids comprising two or more functional groups and at least one linker between at least two functional groups. In some aspects, one of the two or more functional groups is a lipid group, a lipid raft group, a cationic ionizable group, a steric group, a sterol group, a saccharide group, a folate group, a GalNAc group, a oligo peptide group, or a oligo nucleotide group. In some aspects, the linker is covalently linked to two or more functional groups. In some aspects, the two or more functional groups comprise a lipid raft group and a cationic ionizable group. In one aspect, the two or more functional groups comprise a sterol group and a cationic ionizable group. In one aspect, the two or more functional groups comprise a saccharide group and at least one of a sterol group and a PEG group.

In one aspect, a modular lipid is derived from viral envelope lipids with saccharide modifications. In one aspect, the modular lipid is modified with monosaccharide, disaccharide, oligosaccharide or polysaccharide modifications.

In one aspect, the LNP comprises a modular lipid compound selected from Formulae VIII-IX, wherein Formula VIII is

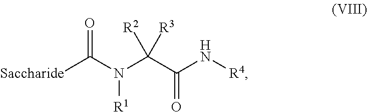

or a salt or isomer thereof;
wherein Formula IX is

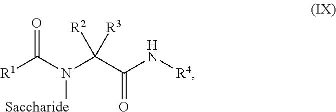

or a salt or isomer thereof;
wherein
each of R¹, R², R³ and R⁴ is independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, poly(ethylene glycol) (PEG),

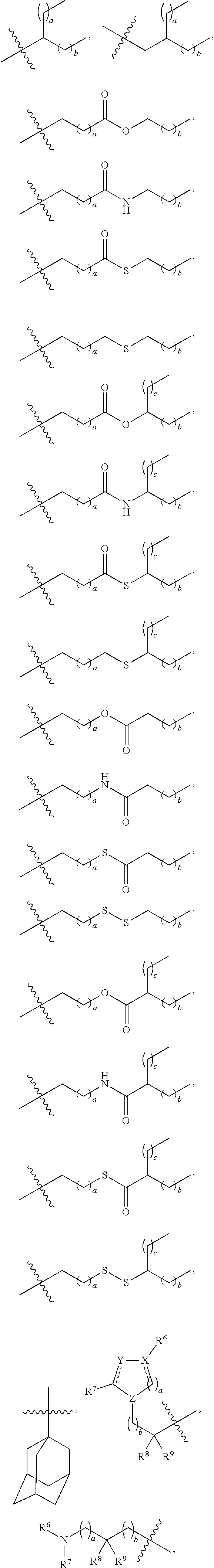

a, b and c are each independently an integer from 0-24;
each of R⁶, R⁷, R⁸ and R⁹ is independently selected from H, C1-C24 alkyl, C1-C24 alkenyl, C1-C24 alkynyl, substituted alkyl, substituted alkenyl, substituted alkynyl, substituted acyl,

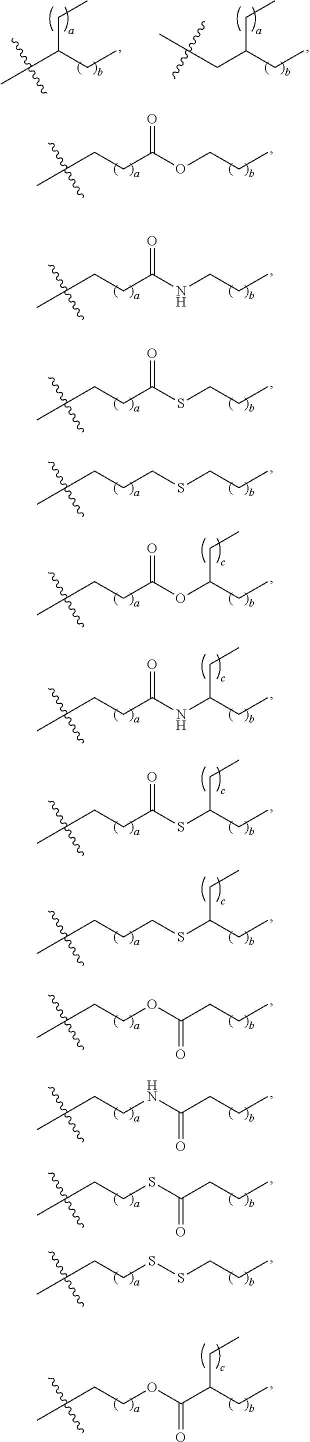

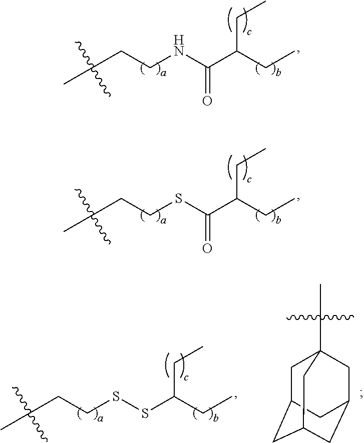

a, b and c are each independently an integer from 0-24;
each X is independently selected from CH or N;
each Y is independently selected from CH₂, NH, O, or S;
each Z is independently selected from CH or N; and
each saccharide is independently selected from monosaccharides, disaccharides, oligosaccharides, and polysaccharides.

Monosaccharides useful in the composition of the disclosure include trioses (ketotriose, aldotriose), tetroses (ketotetrose, aldotetroses), pentoses (ribulose, xylulose, ribose, arabinose, xylose, lyxose, deoxyribose), hexoses (psicose, fructose, sorbose, tagatose, allose, altrose, glucose, mannose, fucose, fuculose, rhamnose, heptose, octose, nonose, gulose, idose, galactose, talose), sedoheptulose. Disaccharides useful in the composition of the disclosure include sucrose, lactose, maltose, trehalose, turanose, cellobiose. Oligosaccharides useful in the composition of the disclosure include raffinose, melezitose, maltotriose, acarbose, stachyose, fructooligosaccharide, galactooligosaccharides, mannanoligosaccharides. Polysaccharides useful in the composition of the disclosure include ployglycitol, n-acetylglucosamine, chitin.

In one aspect, the LNP comprises a modular lipid compound selected from Formulae X-XX,
wherein Formula X is

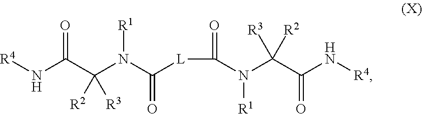

or a salt or isomer thereof,
wherein Formula XI is

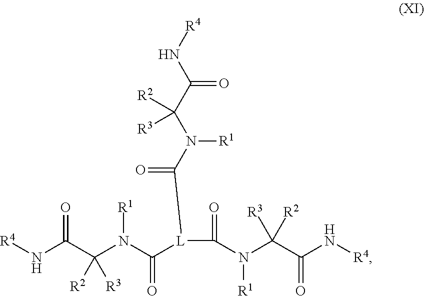

or salt or isomer thereof,
wherein Formula XII is

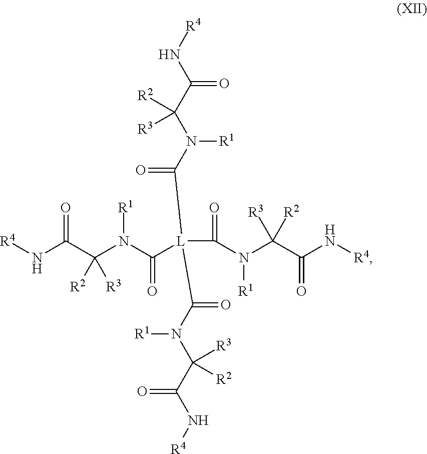

or salt or isomer thereof,
wherein Formula XIII is

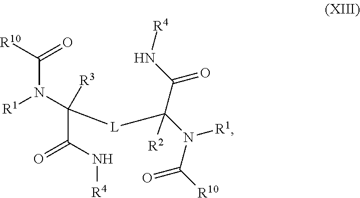

or salt or isomer thereof;
wherein Formula IXX is

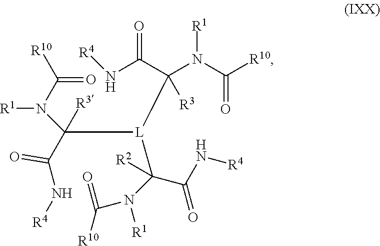

or a salt or isomer thereof,
wherein Formula XX is

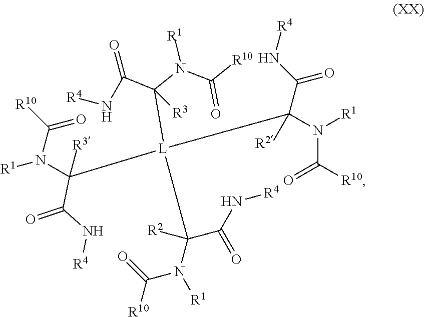

or a salt or isomer thereof, wherein each R¹, R⁴, and R¹⁰, is independently selected from C₂-C₂₄ alkyl, C₂-C₂₄ alkenyl, C₂-C₂₄ alkynyl, substituted C₂-C₂₄ alkyl, substituted alkenyl, substituted alkynyl, substituted acyl, substituted carbocyclyl, substituted heterocyclyl, substituted aryl, substituted heteroaryl, poly(ethylene glycol) (PEG),

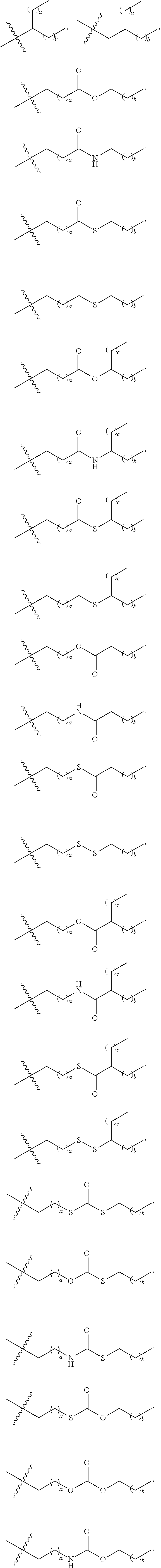

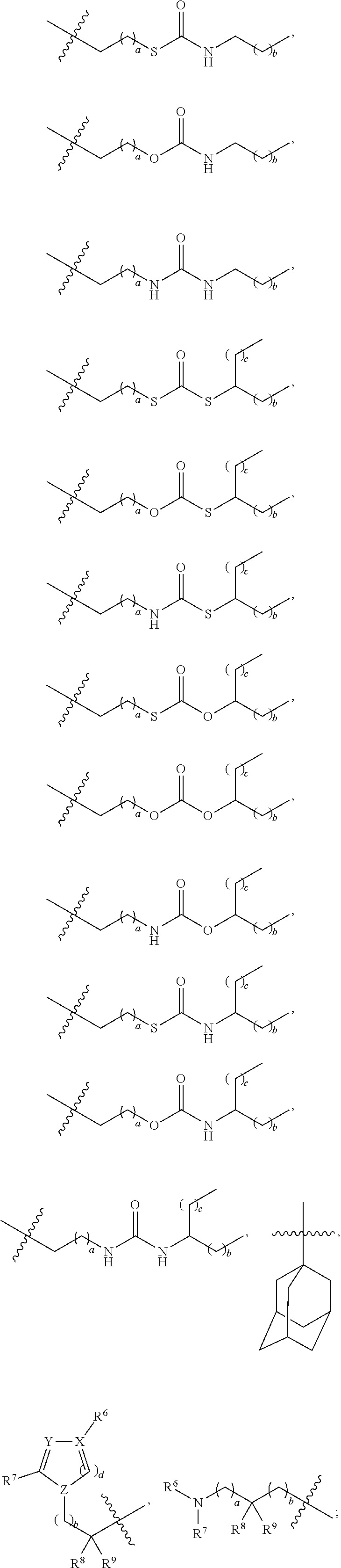

wherein each R², R^{2′}, R³ and R^{3′} is independently selected from H, C₁-C₂₄ alkyl, C1-C24 alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, poly(ethylene glycol) (PEG) and

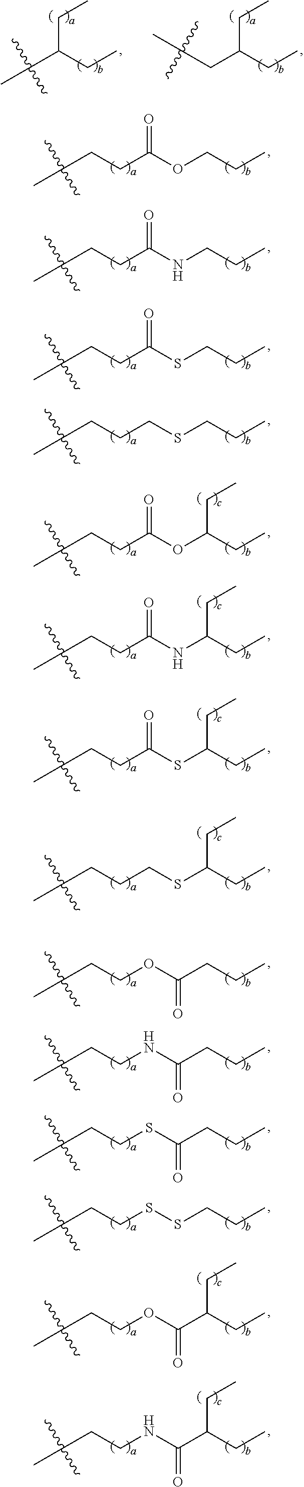

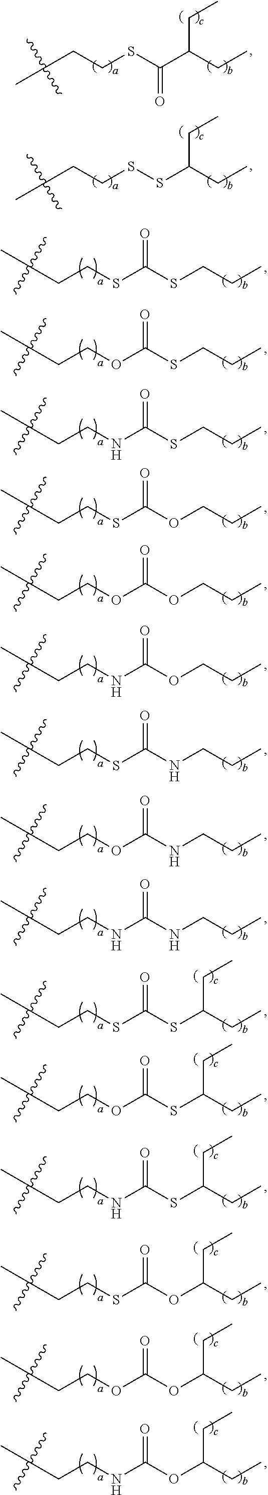

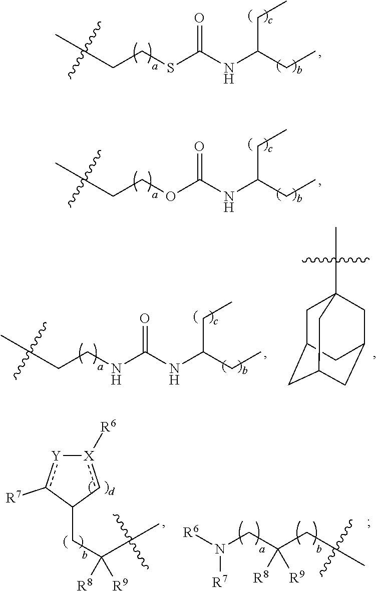

wherein each L is independently selected from alkyl, alkenyl, alkynyl, substituted alkyl, substituted alkenyl, substituted alkynyl, substituted acyl, substituted carbocyclyl, substituted heterocyclyl, substituted aryl, substituted heteroaryl, poly(ethylene glycol) (PEG) and

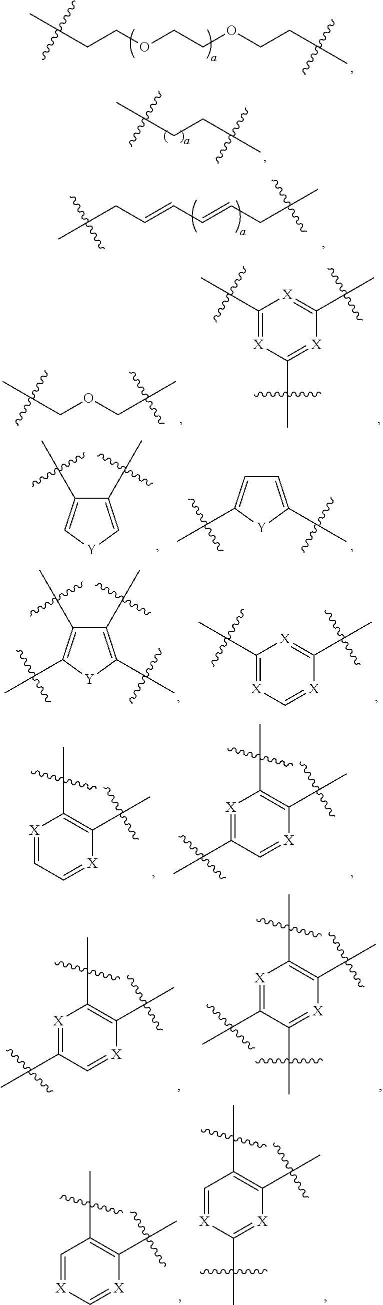

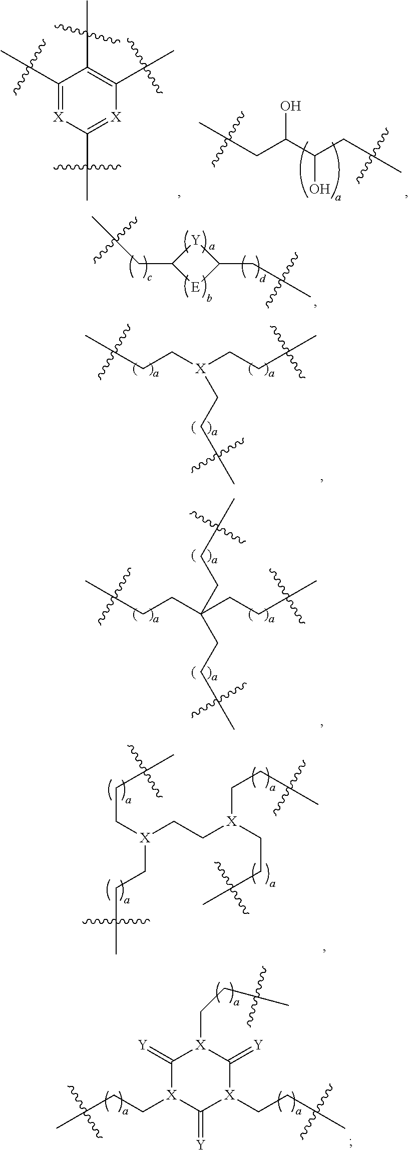

wherein R⁶, R⁷, R⁸ and R⁹ are each independently selected from H, C₁-C₂₄ alkyl, C₁-C₂₄ alkenyl, C₁-C₂₄ alkynyl, substituted alkyl, substituted alkenyl, substituted alkynyl, substituted acyl,

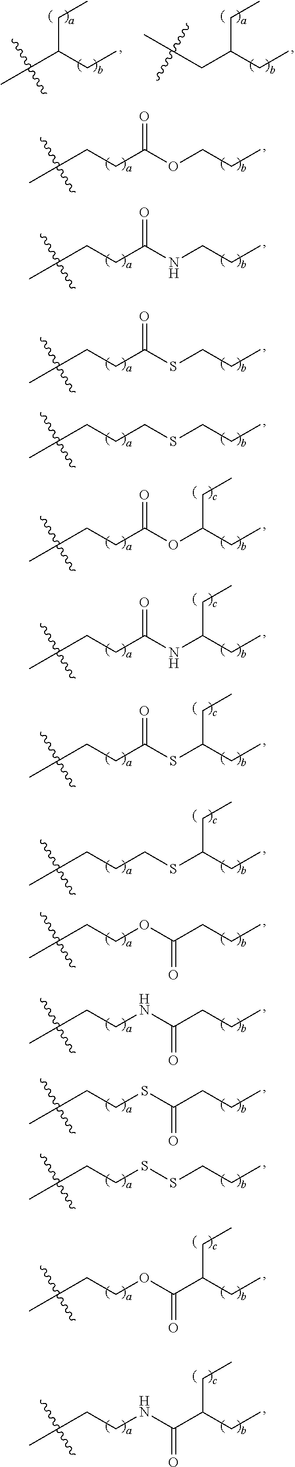

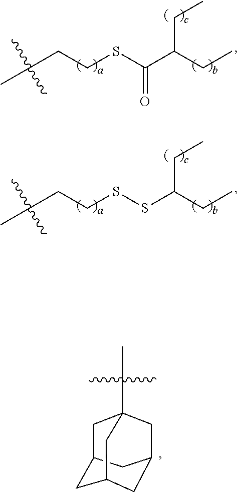

a, b, c and d are each independently an integer from 0-24;
each E is independently selected from CH₂, NH, O, or S;
each X is independently selected from CH or N;
each Y is independently selected from CH₂, NH, O, or S; and
each Z is independently selected from CH or N.

In some aspects, the modular lipid is:

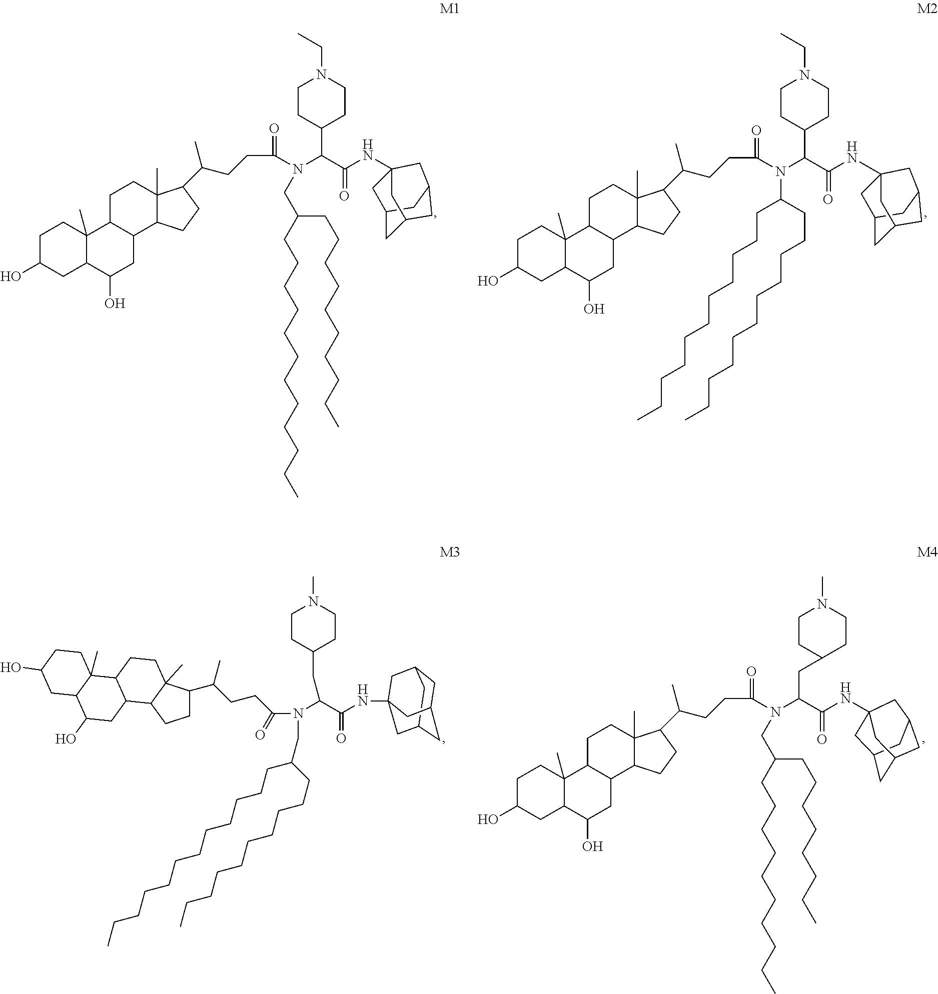 [SMILES: CCCCCCCCCCCCC(CCCCCCCCCC)CN(C(=O)CCC(C)C1CCC2C3CC(O)C4CC(O)CCC4(C)C3CCC12C)C(C(=O)NC12CC3CC(CC(C3)C1)C2)C1CCN(CC)CC1 ; CCCCCCCCCCCC(CCCCCCCCCCC)N(C(=O)CCC(C)C1CCC2C3CC(O)C4CC(O)CCC4(C)C3CCC12C)C(C(=O)NC12CC3CC(CC(C3)C1)C2)C1CCN(CC)CC1 ; CCCCCCCCCCCCC(CCCCCCCCCC)CN(C(=O)CCC(C)C1CCC2C3CC(O)C4CC(O)CCC4(C)C3CCC12C)C(CC1CCN(C)CC1)C(=O)NC12CC3CC(CC(C3)C1)C2 ; CCCCCCCCCCC(CCCCCCCC)CN(C(=O)CCC(C)C1CCC2C3CC(O)C4CC(O)CCC4(C)C3CCC12C)C(CC1CCN(C)CC1)C(=O)NC12CC3CC(CC(C3)C1)C2]

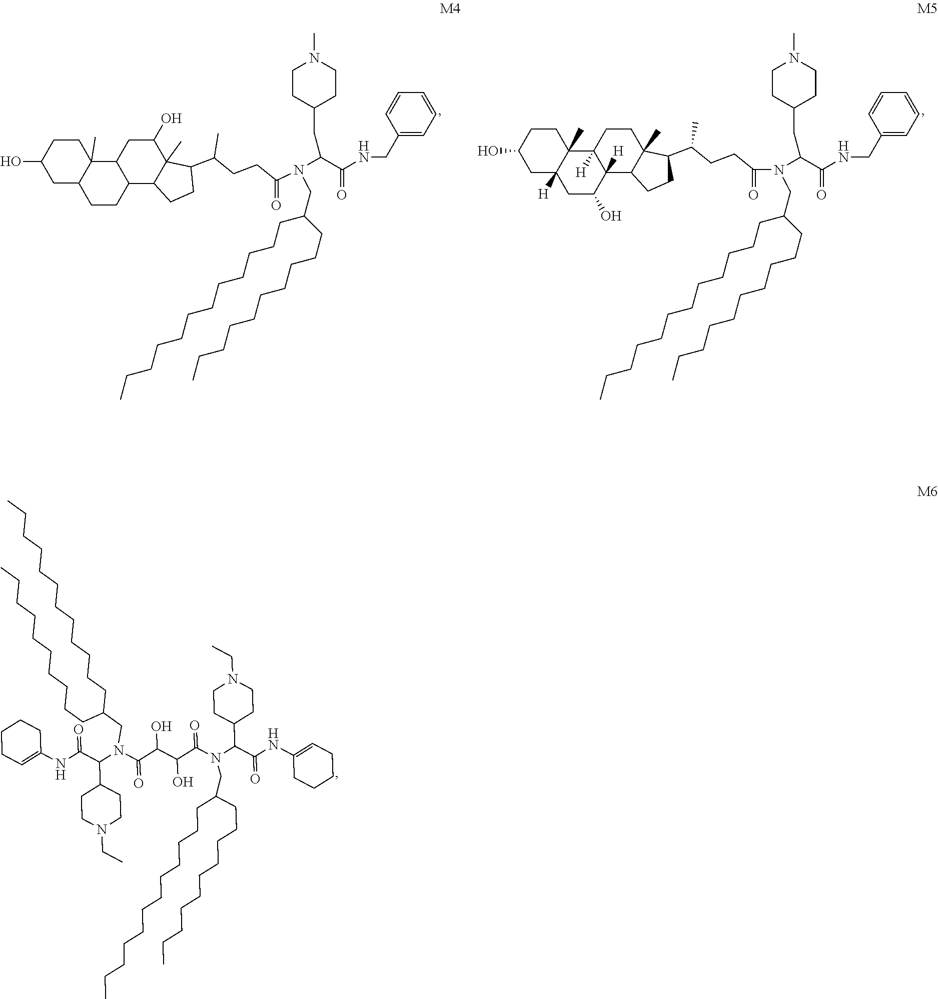

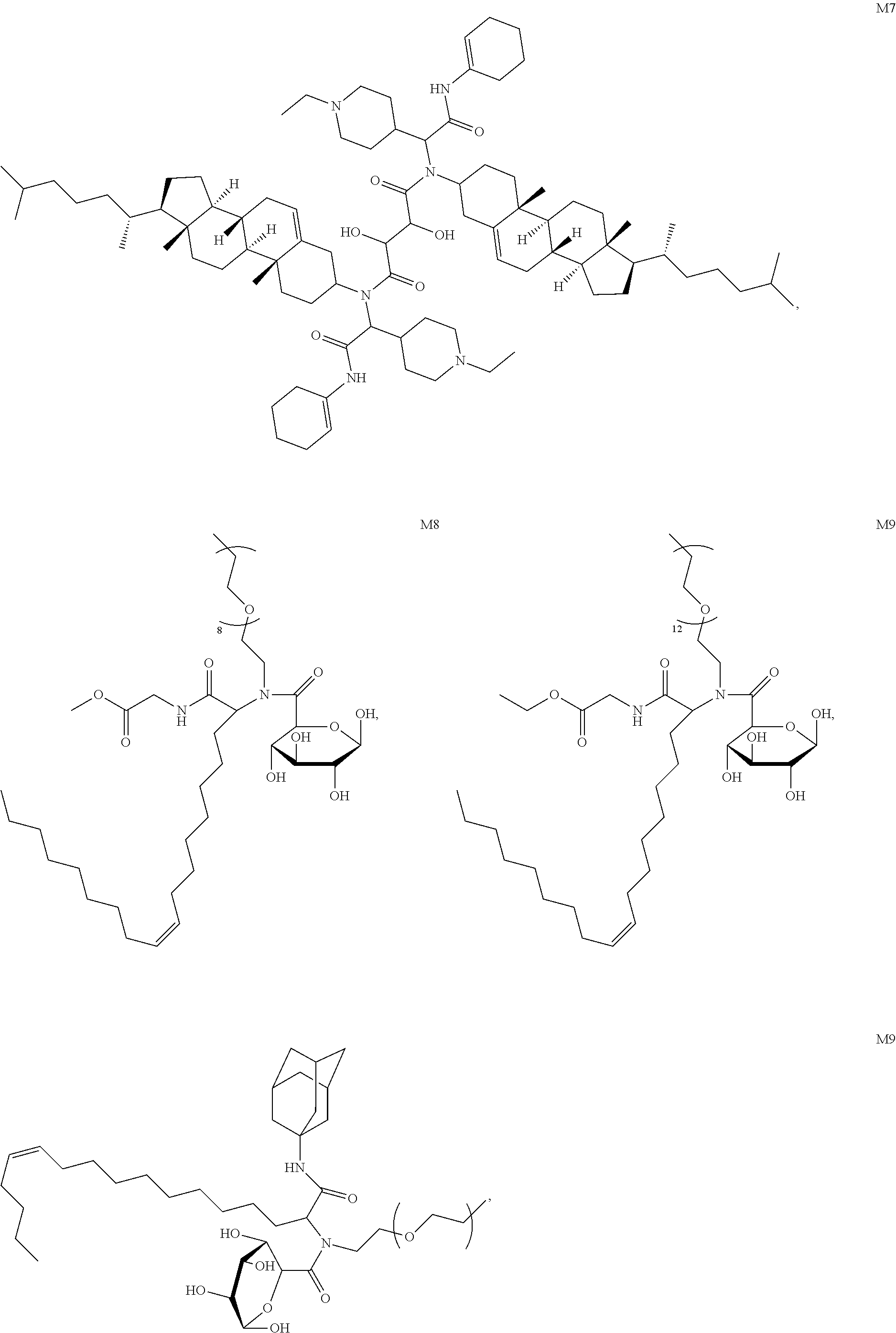

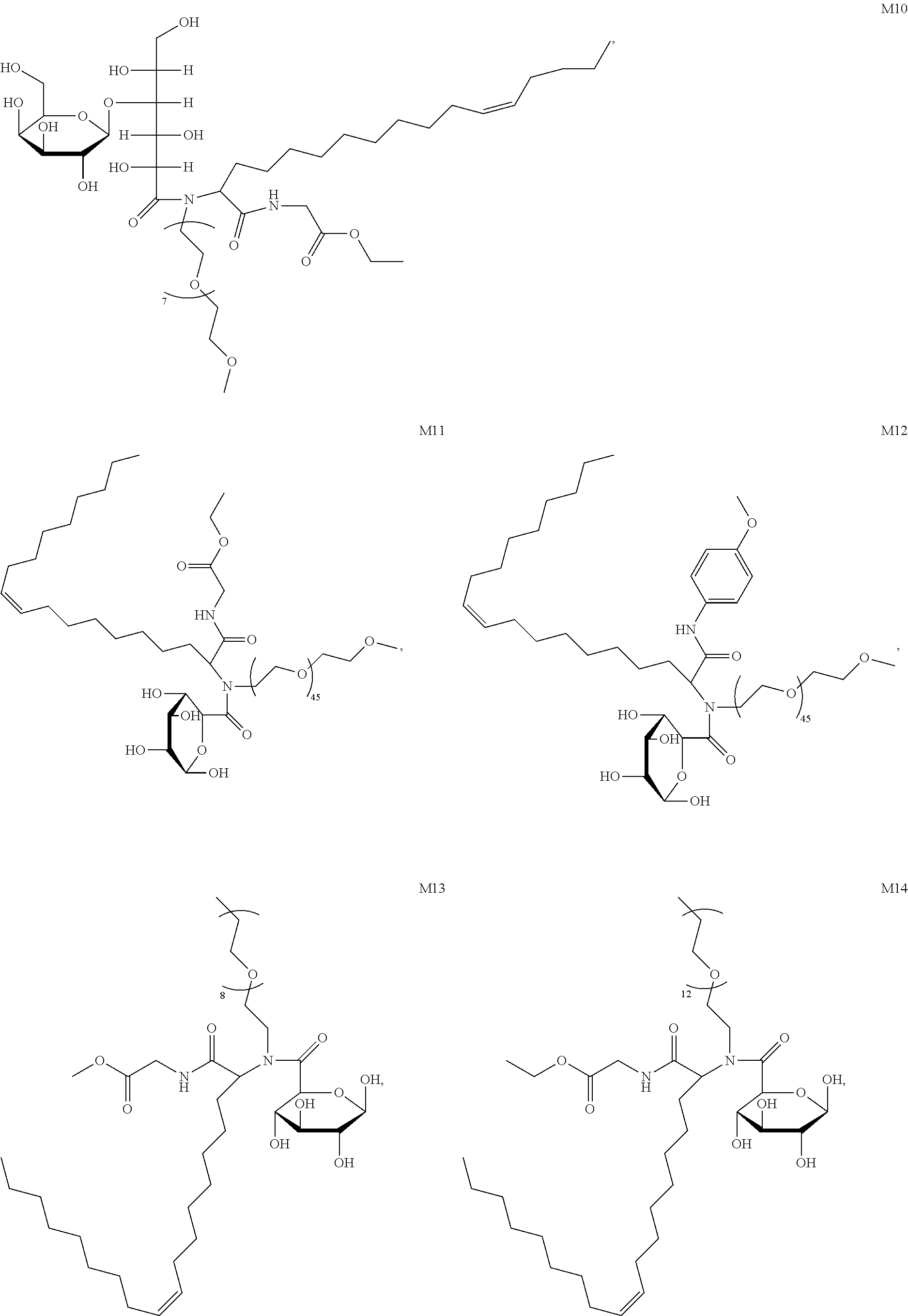

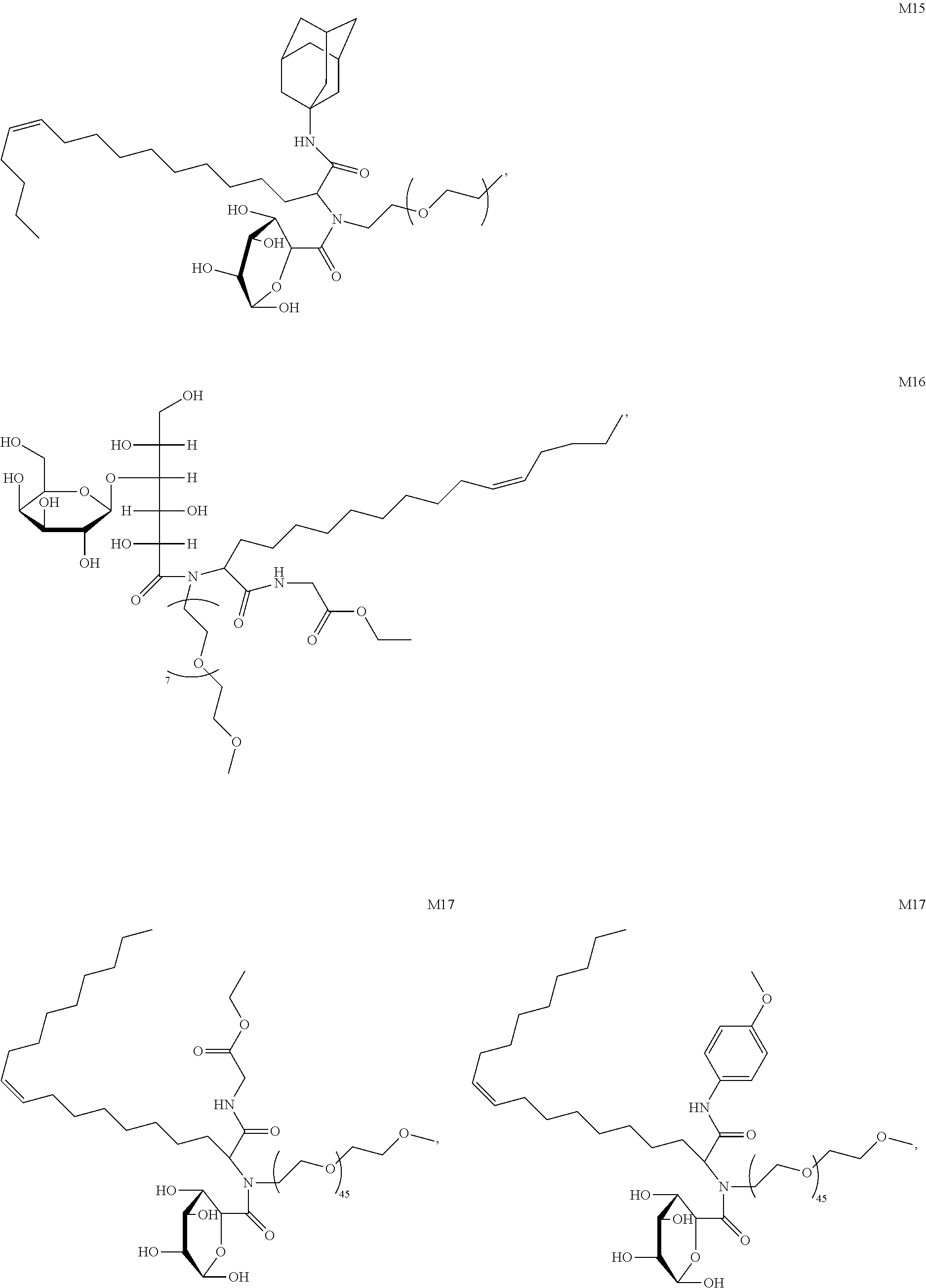

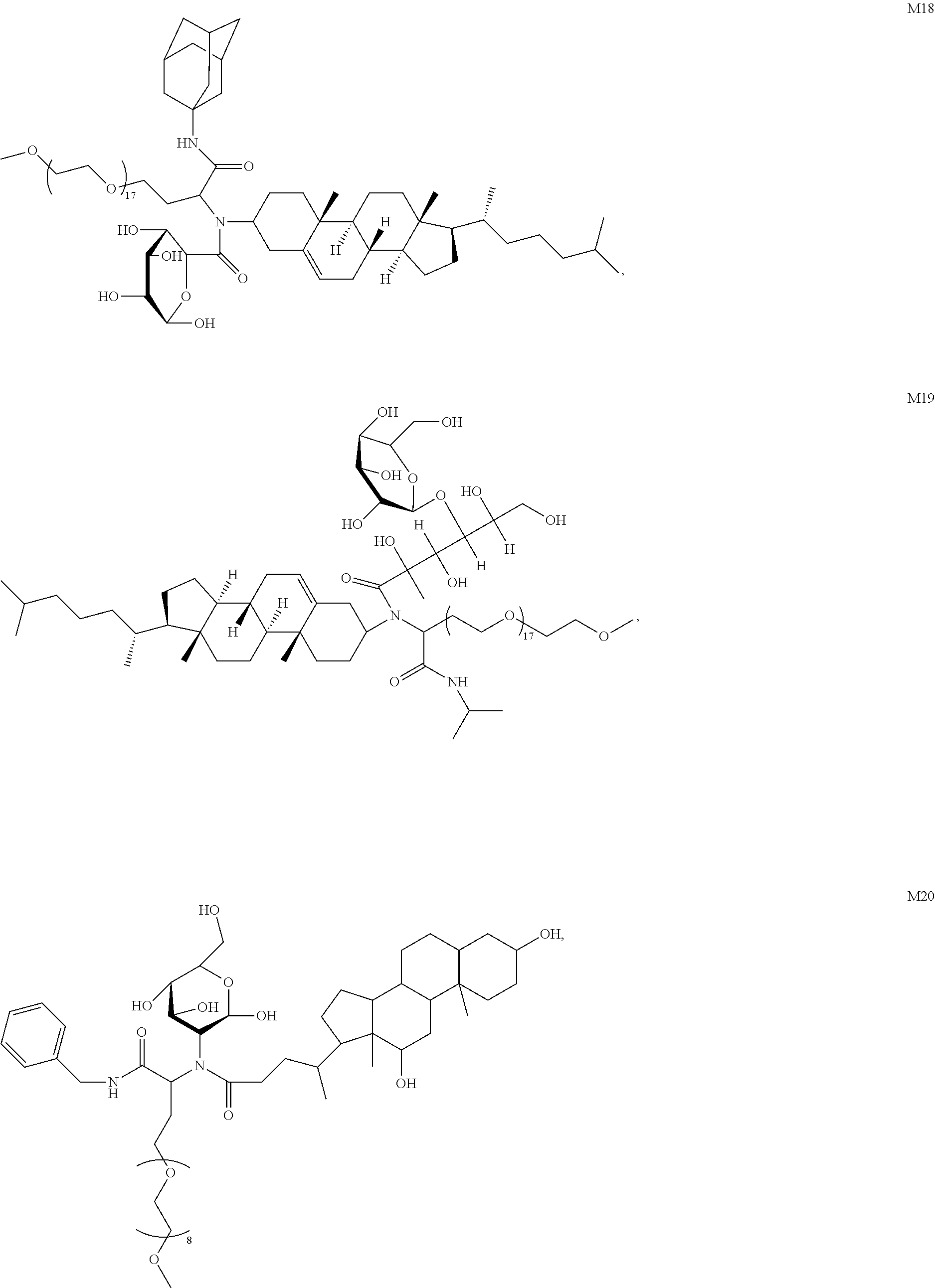

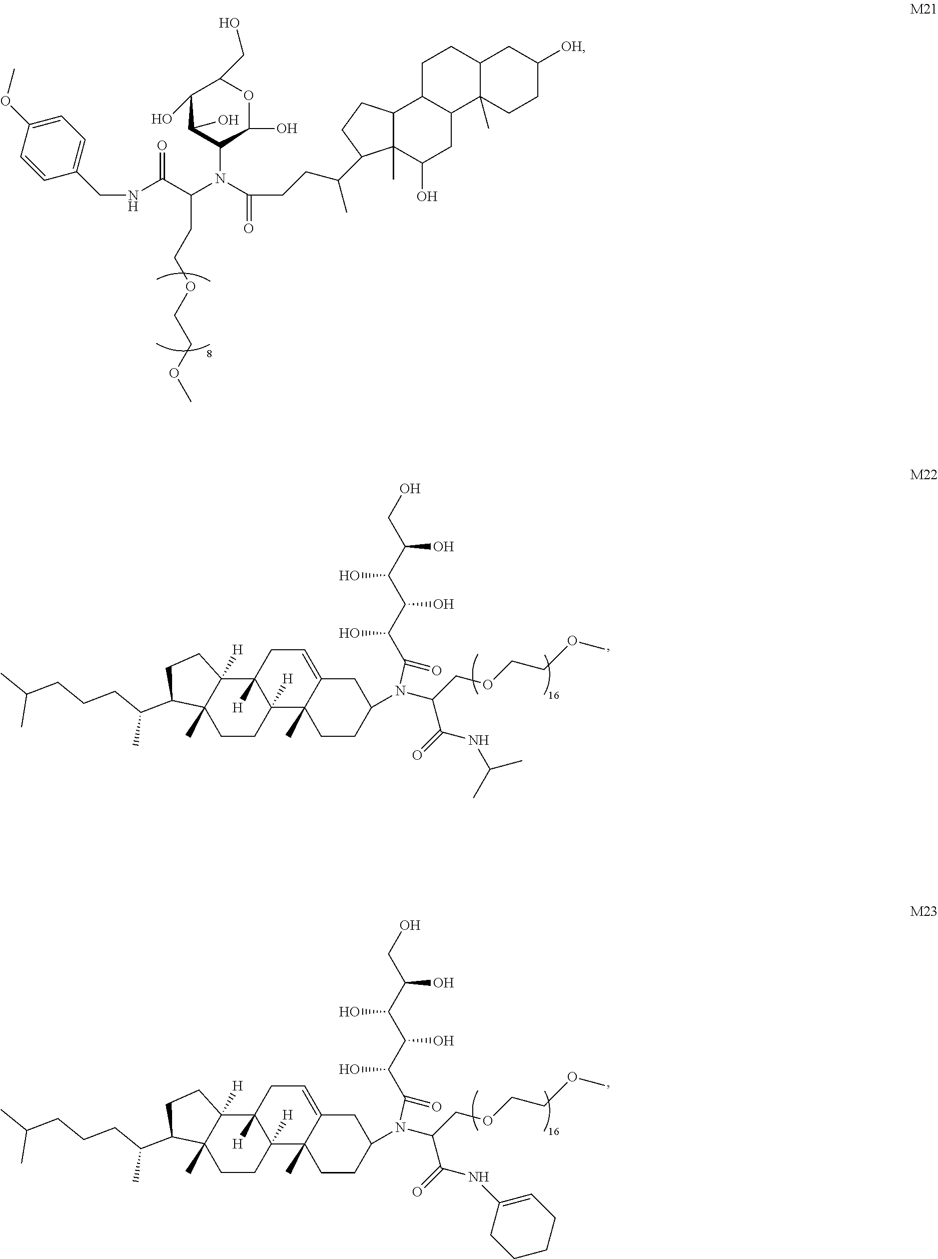

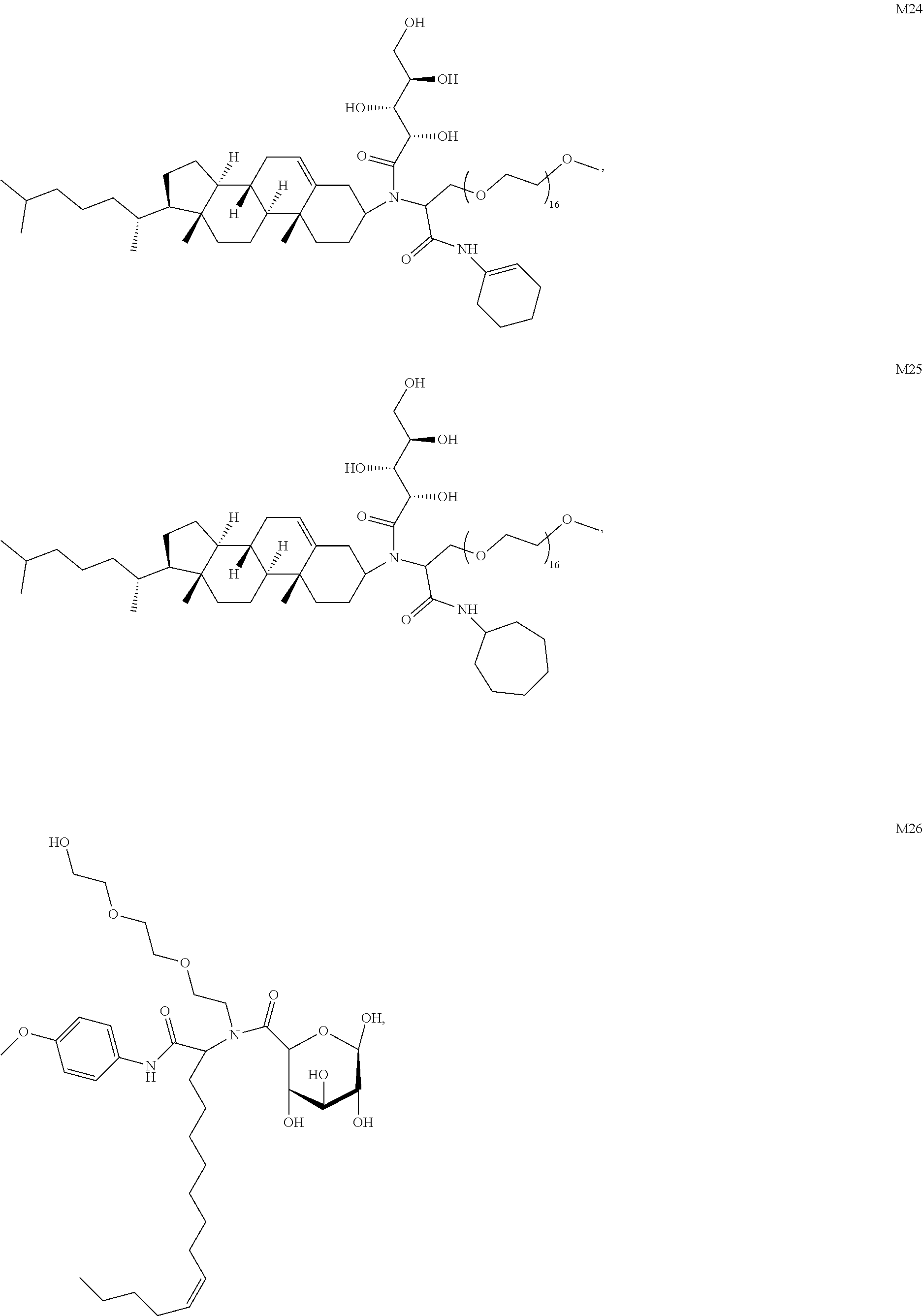

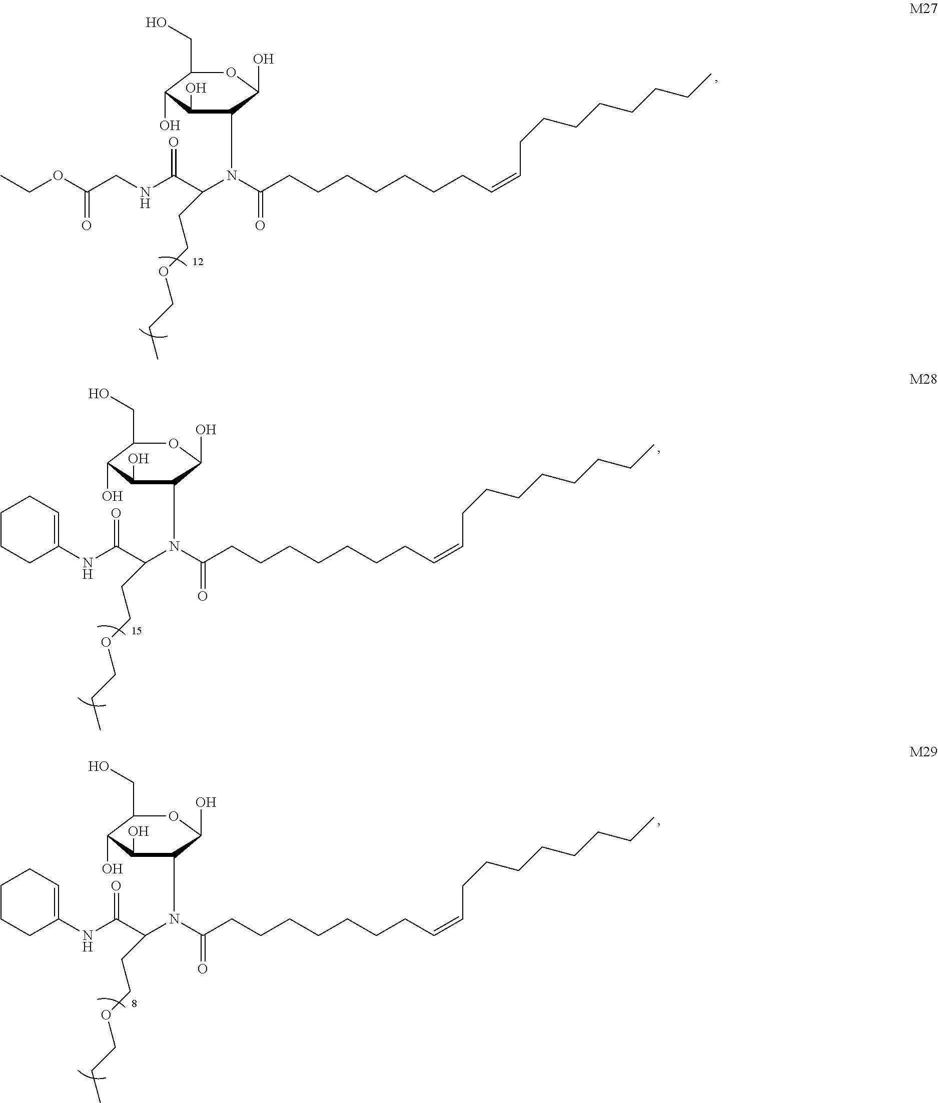

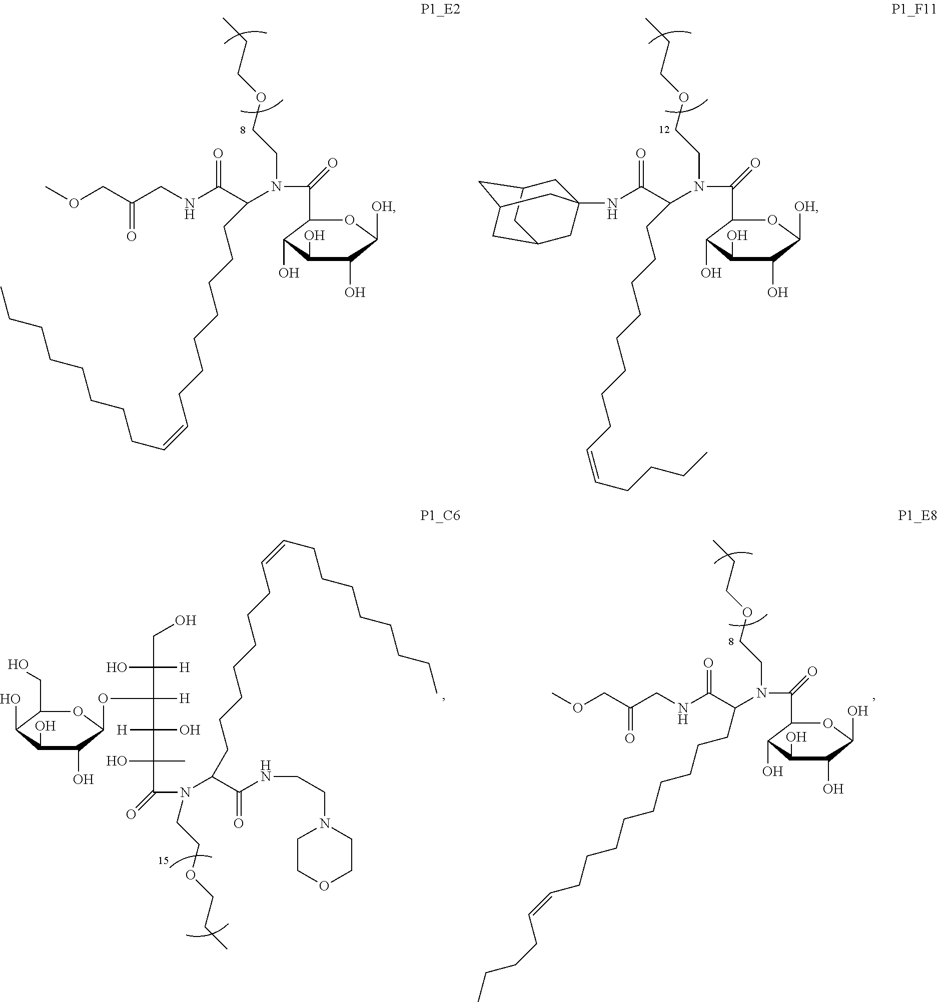

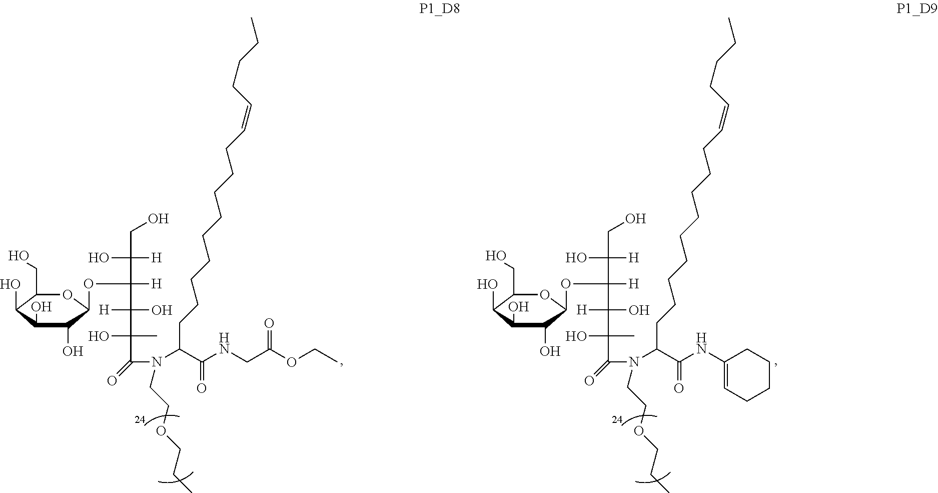

or a salt or isomer thereof.

### Adjuvants

In some aspects, a nanoparticle composition that includes one or more lipids described herein may further include one or more adjuvants, e.g., Glucopyranosyl Lipid Adjuvant (GLA), CpG oligodeoxynucleotides (e.g., Class A or B), poly(I.C), aluminum hydroxide, and Pam3CSK4.

### Biologically Active Agents

Payload delivery systems encapsulate a payload. The payload may comprise one or more biologically active agents, which may be a therapeutic, diagnostic or prophylactic. The disclosure features methods of delivering a biologically active agent to a cell or organ and treating a disease or disorder in a subject in need thereof comprising administering to a subject and/or contacting a cell with a payload delivery system including a biologically active agent.

A biologically active agent may be a substance that, once delivered to a cell or organ, brings about a desirable therapeutic change in the cell, organ, or other bodily tissue or system. Such species may be useful in the treatment of one or more diseases, disorders, or conditions.

In some aspects, the payload delivery system of the disclosure encapsulate more than one biologically active agent as its payload. In some aspects, at least one of the biologically active agents of the payload is a small molecule drug useful in the treatment of a particular disease, disorder, or condition. Examples of drugs useful in the nanoparticle compositions include, but are not limited to, antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cytosine arabinoside, anthracyclines, alkylating agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetalol), antihypertensive agents (e.g., clonidine and hydralazine), anti-depressants (e.g., imipramine, amitriptyline, and doxepin), anti-convulsants (e.g., phenytoin), antihistamines (e.g., diphenhydramine, chlorpheniramine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

In some aspects, at least one of the biologically active agents of the payload is a cytotoxin, a radioactive ion, a chemotherapeutic, a vaccine, a compound that elicits an immune response, and/or another therapeutic and/or prophylactic. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol, rachelmycin (CC-1065), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Vaccines include compounds and preparations that are capable of providing immunity against one or more conditions related to infectious diseases such as influenza, measles, human papillomavirus (HPV), rabies, meningitis, whooping cough, tetanus, plague, hepatitis, and tuberculosis and can include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. Compounds eliciting immune responses may include vaccines, corticosteroids (e.g., dexamethasone), and other species.

In some aspects, at least one of the biologically active agents of the payload is a polypeptide or protein. Therapeutic proteins useful in the nanoparticles in the disclosure include, but are not limited to, gentamycin, amikacin, insulin, erythropoietin (EPO), granulocyte-colony stimulating factor (G-CSF), granulocyte-macrophage colony stimulating factor (GM-CSF), Factor VIR, luteinizing hormone-releasing hormone (LHRH) analogs, interferons, heparin, Hepatitis B surface antigen, typhoid vaccine, and cholera vaccine.

In some aspects, at least one of the biologically active agents of the payload is a polynucleotide or nucleic acid (e.g., ribonucleic acid or deoxyribonucleic acid). The term “polynucleotide,” in its broadest sense, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, gRNA, shRNA, siRNAs, shRNAs, miRNAs, tRNA, antisense RNAs, ribozymes, catalytic DNA, RNAs that induce triple helix formation, aptamers, vectors, etc. In some aspects, at least one of the first nucleic acid construct or the second nucleic acid construct is a RNA or DNA molecule.

In some aspects, the payload comprise to nucleic acid constructs. In some aspects, at least one of the first nucleic acid construct or the second nucleic acid construct is a DNA construct. In some aspects, at least one of the first nucleic acid construct or the second nucleic acid construct is a double stranded DNA (dsDNA) construct. In some aspects, at least one of the first nucleic acid construct or the second nucleic acid construct is a single stranded DNA (ssDNA) construct. In some aspects, at least one of the first nucleic acid construct or the second nucleic acid construct is a linear nucleic acid molecule. In some aspects, at least one of the first nucleic acid construct or the second nucleic acid construct is a circular nucleic acid molecule. RNAs useful in the compositions and methods described herein can be selected from the group consisting of, but are not limited to, shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and mixtures thereof. In some aspects, the first nucleic acid construct is a sa-mRNA molecule. In some aspects, the second nucleic acid construct is a mRNA molecule.

### Polynucleotides and Nucleic Acids

In some aspects, the payload delivered in a payload delivery system of the disclosure is a first nucleic acid construct encoding a sa-mRNA encoding at least one gene of interest (GOI) or a plurality of GOIs; and a second nucleic acid construct encoding an mRNA encoding at least one virus structural protein.

The amount of a biologically active agent in a payload delivery system may depend on the size, composition, desired target and/or application, or other properties of the payload delivery system as well as on the properties of the biologically active agent. For example, the amount of a nucleic acid useful in a payload delivery system may depend on the size, sequence, and other characteristics of the nucleic acid. The relative amounts of a biologically active agent and other elements (e.g., lipids) in a payload delivery system may also vary. In some aspects, the wt/wt ratio of the lipid component to a biologically active agent in a payload delivery system may be from about 1:1 to about 60:1, such as 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a biologically active agent may be from about 1:1 to about 4:1. In certain aspects, the wt/wt ratio is about 20:1. In certain aspects, the wt/wt ratio is about 60:1. The amount of a biologically active agent in a payload delivery system may, for example, be measured using absorption spectroscopy (e.g., ultraviolet-visible spectroscopy).

Self-Amplifying mRNA

Sa-mRNAs of the disclosure have the ability to self-replicate in cells and, thus, can be used to induce expression of encoded gene products, such as proteins (e.g., antigens) and regulatory structures (e.g. siRNA, miRNA, saRNA, tRNA, and lincRNA) encoded by the sa-mRNA. In addition, sa-mRNAs are generally based on the genome of an RNA virus (e.g. a Group IV positive single strand RNA virus), and therefore are foreign nucleic acids that can stimulate the innate immune system (e.g. induce an interferon response). This can lead to undesired consequences and safety concerns, such as rapid inactivation and clearance of the RNA, injection site irritation and/or inflammation and/or pain.

The sa-mRNAs of the present disclosure contain modified structures and have reduced capacity to stimulate the innate immune system, which will lead to rapid decay of the sa-mRNA and its associated gene products. Rapid decay of the sa-mRNA and its associated gene products will lead to increased frequency of administration, which is associated with safety concerns and reduced therapeutic efficacy. Thus one aspect of the invention is sa-mRNAs that have reduced cytotoxic effects on the host cell or subject. This provides for enhanced safety of the sa-mRNAs of the present disclosure and provides additional advantages. For example, an advantage of a sa-mRNA with low cytotoxicity allows for administration of a large dose of the sa-mRNAs to produce high expression levels of the encoded gene product with reduced risk of undesired effects, such as injection site irritation and or pain. Because CATP can increase therapeutic payloads dramatically, one can reduce the therapeutic dosage to reduce the toxicity. In certain embodiments, toxicity is reduced at least about 20%, at least about 25%, at least about 30%, at least about 35%, at least about 40%, at least about 50%, at least about 60%, at least about 70%, at least about 75%, at least about 80%, or at least about 90%, for example about 20% to about 90%, about 20% to about 75%, about 30% to about 50%, or about 45% to about 55%.

One suitable system for producing a sa-mRNA of the present disclosure is to use an alphavirus-based RNA replicon. Alphavirus-based replicons are positive (+)-single stranded replicons that can be translated after delivery to a cell to give a replicase (or replicase-transcriptase). The replicase is translated as a polyprotein which auto-cleaves to provide a replication complex, comprising plurality of non-structural replicase domain sequences, which creates genomic (−)-strand copies of the (+)-strand delivered RNA. These (−)-strand transcripts can themselves be transcribed to give further copies of the (+)-stranded parent RNA and also to give an mRNA transcript which encodes the desired gene product. Translation of the subgenomic transcript thus leads to in situ expression of the desired gene product by the infected cell.

Whereas natural alphavirus genomes encode structural proteins in addition to the non-structural replicase, in one aspect, an alphavirus based sa-mRNA does not encode alphavirus structural proteins. Thus the sa-mRNA can lead to the production of RNA copies of itself in a cell, but not to the production of RNA-containing alphavirus virions. The inability to produce these virions means that, unlike a wild-type alphavirus, the sa-mRNA cannot perpetuate itself in infectious form. The alphavirus structural proteins which are necessary for perpetuation in wild-type viruses are absent from self-amplifying mRNAs and their place is taken by the GOI, such that the sa-mRNA transcript encodes the desired gene product rather than the structural alphavirus virion proteins.

The sa-mRNAs described herein may be engineered to express multiple GOI, from two or more coding regions, thereby allowing co-expression of proteins and or regulatory structures, such as a two or more antigens together with cytokines or other immunomodulators, which can enhance the generation of an immune response. Such a sa-mRNA might be particularly useful, for example, in the production of various gene products (e.g., proteins) at the same time.

In one aspect, the payload delivered in a payload delivery system of the disclosure include a first nucleic acid construct encoding a sa-mRNA encoding at least one gene of interest (GOI) or a plurality of GOIs; and a second nucleic acid construct encoding an mRNA encoding at least one virus structural protein.

In on aspect, the first nucleic acid construct comprises an operably linked nucleic acid sequence comprising from 5′ to 3′:
[5′ UTR]-[nsP]-[SGP]-[GOI]-[3′ UTR]-[Poly A]
wherein, 5′ UTR is a 5′ untranslated region, nsP is a plurality of non-structural replicase domain sequences, SGP is a subgenomic promoter, GOI is one gene of interest or a plurality of genes of interest, 3′ UTR is a 3′ untranslated region, and Poly A is a poly A tail. In some aspects, the first nucleic construct comprise more than one GOI operably linked to one or more SGP. In some aspects, each GOI is linked to a different SGP.

In some aspects, the first nucleic acid construct comprises an operably linked nucleic acid sequence comprising from 5′ to 3′:
[5′ UTR]-[nsP]—[ORF]-[3′ UTR]-[PolyA]
wherein 5′ UTR is a 5′ untranslated region, nsP is at least one non-structural replicase domain sequence, ORF is two or more open reading frames, 3′ UTR is a 3′ untranslated region, and Poly-A is a 3′ poly-A tail, wherein the ORF comprises at least two SGP operably linked to at least one GOI.

In some aspects, the operably linked nucleic acid sequence further comprises one or more linkers. In some aspects, the one or more linkers are independently selected from a sequence comprising any one of SEQ ID NOs: 27-37.

In one aspect, the first nucleic acid construct comprises an operably linked nucleic acid sequence comprising from 5′ to 3′:
[5′ UTR]-[nsP]-[SGP]-[L]-[GOI]-[L]-[3′ UTR]-[PolyA]
wherein, 5′ UTR is a 5′ untranslated region, nsP is a plurality of non-structural replicase domain sequences, L is a linker independently selected from a sequence comprising any one of SEQ ID NOs: 27-37, SGP is a subgenomic promoter, GOI is one or more genes of interest, 3′ UTR is a 3′ untranslated region, and poly-A is a poly-A tail.

In some aspects, the plurality of non-structural replicase domain sequences are obtained from a Group IV positive single strand RNA virus selected from Picornaviridae, Togaviridae, Coronaviridae, Hepeviridae, Caliciviridae, Flaviviridae, or Astroviridae. In some aspects, the plurality of non-structural replicase domain sequences are obtained from an alphavirus selected from Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, or Buggy Creek virus. In some aspects, the plurality of non-structural replicase domain sequences are alphavirus nonstructural proteins 1-4 (nsP1-4). In some aspects, the plurality of non-structural replicase domain sequences are obtained from the TC-83 strain of VEE. In some aspects, the nsP is SEQ ID NO: 19.

In some aspects, the 5′ UTR is selected from a sequence comprising any one of SEQ ID NO: 15, 16, 17, 18, 71, or 72. In some aspects, the 3′ UTR comprises SEQ ID NO: 21, 22, 69 or 70.

In some aspects, the GOI encodes a therapeutic polypeptide, a prophylactic polypeptide, a diagnostic polypeptide, an antigen, a non-coding gene that encodes regulatory structure, or a reporter. In some aspects, the regulatory structure is selected from small interfering RNA (siRNA), micro-RNA (miRNA), an asymmetrical interfering RNA (aiRNA), a Dicer-substrate RNA (dsRNA), a small hairpin RNA (shRNA), small activating RNA (saRNA), transfer RNA (tRNA), or long intergenic non-coding (lincRNA).

In some aspects, the GOI encodes a infectious pathogen antigen, an allergen antigen, a tumor antigen, or a combination thereof. In some aspects, the GOI encodes an immunomodulator. In some aspects, the immunomodulator comprises a cytokine, a chemokine, anti-neoplastic fusion protein, single domain antibody, bispecific antibody, multispecific antibody, a stem cell growth factor, a lymphotoxin, a stem cell growth factor, an erythropoietin, a thrombopoietin, an interleukin, an interferon, a fusion protein, a colony stimulating factor, or a combination thereof. In some aspects, the interleukin comprises IL-1, IL-la, IL-10, IL-1Ra, IL-2, IL-3, IL-4, IL-6, IL-7, IL-9, IL-10, IL-12, IL-13, IL-15, IL-3, IL-5, IL-6, IL-11, IL-12, IL-10, IL-20, IL-14, IL-16, IL-17, IL-18 or IL-21. In some aspects, the interleukin is selected from SEQ ID NOs: 4-9 or 45-55. In some aspects, the interferon is an interferon-α, -β and -γ. In some aspects, the colony stimulating factor is a granulocyte-colony stimulating factor (G-CSF), or a macrophage colony stimulating factor (M-CSF), or a granulocyte macrophage-colony stimulating factor (GM-CSF), a transforming growth factor beta, a transforming growth factor alpha, a bone morphogenetic protein, a fibroblast growth factor, an insulin-like growth factor, a neurotrophic factor, thrombopoietin.

In some aspects, the GOI comprises SEQ ID NO: 4, SEQ ID NO: 5, SEQ ID NO: 6, SEQ ID NO: 7, SEQ ID NO: 8, SEQ ID NO: 9, SEQ ID NO: 23, SEQ ID NO: 24, SEQ ID NO: 25, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO. 101, or SEQ ID NO: 55.

In some aspects, the SGP is a viral promoter that is recognized by viral RNA dependent RNA polymerase. In some aspects, the SGP is SEQ ID NO: 1, 2 or 3.

In some aspects, the first nucleic acid construct comprises SEQ ID NO: 41, 42, 73, 74, 75, 76, 77, 78, 79, 80, or 81.

In one aspect, the first nucleic acid construct is a sa-mRNA, wherein the sa-mRNA is SEQ ID NO: 73 comprising at least one substitution selected from:
a substitution of C with A at position 2623;
a substitution of T with C at position 3309;
a substitution of A with C at position 3493;
a substitution of A with G at position 3867;
a substitution of A with G at position 4674; or
a substitution of A with G at position 5897.

In some aspects, the first nucleic acid construct is a sa-mRNA. The sa-mRNAs of the present disclosure may be produced from a nucleic acid template in the form of recombinant DNA expression vectors, RNA replicons or plasmids. The nucleic acid template of the present disclosure encodes two expression units comprising: i) an origin of replication sequence (Ori); ii) a first expression unit encoding a first nucleotide sequence that is operably linked to a first promoter; and iii) a second expression unit encoding a second nucleotide sequence that is operably linked to a second promoter, wherein the first expression unit encodes a selectable marker and the second expression unit encodes a sa-mRNA.

In some aspects, the second promoter is a promoter that drives transcription of the encoded self-amplifying mRNA using the second expression unit as a template for in vitro transcription of nucleic acid, e.g. mRNA. Suitable promoters include, for example, T7 promoter, T3 promoter, SV40 promoter, SP6 promoter, T5 promoter, β-lactamase promoter, E. coli galactose promoter, arabinose promoter, alkaline phosphatase promoter, tryptophan (trp) promoter, lactose operon (lac) promoter, lacUV5 promoter, trc promoter, tac promoter, Klebsiella phage promoter (K11 RNAP) and the like, or mutants of these promoters. In some aspects, the SP6 promoter comprises SEQ ID NO: 82 (ATTTAGGTGACAC). In some aspects, the K11 RNAP promoter comprises SEQ ID NO: 83 (AATTAGGGCACAC). A sa-mRNA can be prepared by transcribing (e.g., in vitro transcription) a DNA that encodes the sa-mRNA using a suitable DNA-dependent RNA polymerase, such as: T7 phage RNA polymerase, SP6 phage RNA polymerase, T3 phage RNA polymerase, T5 phage RNA polymerase, RNA polymerase III, RNA polymerase II, Taq polymerase, Vent polymerase, and the like, or mutants of these polymerases. The transcription reaction will contain nucleotides, including modified nucleotides in some aspects, and other components that support the activity of the selected polymerase, such as a suitable buffer, and suitable salts. In some aspects, nucleotide analogs will be incorporated into a sa-mRNA to, for example, alter the stability of such RNA molecules, to increase resistance against RNases, to establish replication after introduction into appropriate host cells (“infectivity” of the RNA), and/or to induce or reduce innate and adaptive immune responses.

In one aspect, the second promoter is a mutant T7 promoter. In some aspects, a modified T7 promoter comprises at least one insertion at position at the 5′ end of the wildtype T7 promoter nucleotide sequence. The modification may be, for example, insertion of a single guanine (G) at the 5′ end of the wildtype T7 promoter. In some aspects, the mutant T7 promoter comprises SEQ ID NO: 13 (TAATACGACTCACTATAGGATAGG). In some aspects, the wildtype T7 promoter comprises SEQ ID NO: 14 (TAATACGACTCACTATAGG).

In one aspect, the present disclosure relates to a method of obtaining RNA, including mRNA or sa-mRNA, comprising: a) performing an in vitro transcription reaction using an initial amount of a nucleic acid molecule comprising a promoter and a nucleic acid sequence encoding a RNA, and b) producing the RNA by in vitro transcription, using the nucleic acid molecule as a template and a RNA polymerase (e.g., T7 polymerase). In some aspects, the promoter is a mutant T7 promoter or a wildtype T7 promoter. In some aspects, an increased copy number of the RNA is produced when the promoter is a mutant T7 promoter compared to when the promoter is a wildtype T7 promoter.

### Pharmaceutical Compositions

Compositions of the disclosure may be formulated in whole or in part as pharmaceutical compositions. For example, a pharmaceutical composition may include a pharmaceutical composition comprising the composition of the disclosure and a pharmaceutically acceptable carrier. Pharmaceutical compositions may further include one or more pharmaceutically acceptable excipients or accessory ingredients such as those described herein. General guidelines for the formulation and manufacture of pharmaceutical compositions and agents are known in the art, for example, Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a payload delivery system. An excipient or accessory ingredient may be incompatible with a payload delivery system if its combination with the component may result in any undesirable biological effect or otherwise deleterious effect.

In some aspects, the first nucleic acid construct and the second nucleic acid construct are present in the pharmaceutical composition in a 1:100 to 100:1 ratio. In some aspects, the first nucleic acid construct and the second nucleic acid construct are present in the pharmaceutical composition in a 50:1 to 1:50, 40:1 to 1:40, 30:1 to 1:30, 25:1 to 1:25, 20:1 to 1:20, 15:1 to 1:15, 10:1 to 1:10, 9:1 to 1:9, 8:1 to 1:8, 7:1 to 1:7, 7:1 to 1:7, 6:1 to 1:6, 5:1 to 1:5, 4:1 to 1:4, 3:1 to 1:3, 2:1 to 1:1, or 1:1 ratio. In some aspects, the first nucleic acid construct and the second nucleic acid construct are present in the pharmaceutical composition in a 1:1 ratio.

In some aspects, one or more excipients or accessory ingredients may make up greater than 50% of the total mass or volume of a pharmaceutical composition including a composition of the disclosure. For example, the one or more excipients or accessory ingredients may make up 50%, 60%, 70%, 80%, 90%, or more of a pharmaceutical convention. In some aspects, a pharmaceutically acceptable excipient is at least 95%, at least 96%, at least 97%, at least 98%, at least 99%, or 100% pure. In some aspects, an excipient is approved for use in humans and for veterinary use. In some aspects, an excipient is approved by United States Food and Drug Administration. In some aspects, an excipient is pharmaceutical grade. In some aspects, an excipient meets the standards of the United States Pharmacopoeia (USP), the European Pharmacopoeia (EP), the British Pharmacopoeia, and/or the International Pharmacopoeia.

Relative amounts of the one or more payload delivery systems encapsulating biologically active agents, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered.

In certain aspects, the compositions of the disclosure and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. For example, the pharmaceutical composition comprising a composition of the disclosure is a solution that is refrigerated for storage and/or shipment at, for example, about −20° C., −30° C., −40° C., −50° C., −60° C., −70° C., or −80° C. In certain aspects, the disclosure also relates to a method of increasing stability of the compositions of the disclosure by storing the compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.). For example, the compositions of the disclosure and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In one embodiment, the formulation is stabilized for at least 4 weeks at about 4° C. In certain aspects, the pharmaceutical composition of the disclosure comprises a composition of the disclosure disclosed herein and a pharmaceutically acceptable carrier selected from one or more of Tris, an acetate (e.g., sodium acetate), an citrate (e.g., sodium citrate), saline, PBS, and sucrose. In certain embodiments, the carrier may be at a concentration of 1-100 mM (e.g., including but not limited to any numerical value or range within the range of 1-100 mM such as 1, 2, 3, 4, . . . 97, 98, 99, 100, 10-90 mM, 20-80 mM, 30-70 mM and so on).

In certain aspects, the pharmaceutical composition of the disclosure has a pH value between about 5 and 8 (e.g., 5, 5.5, 6, 6.5, 6.8 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises a composition of the disclosure disclosed herein, Tris, saline and sucrose, and has a pH of about 7.5-8, which is suitable for storage and/or shipment at, for example, about −20° C. For example, a pharmaceutical composition of the disclosure comprises one or more compositions of the disclosure disclosed herein and PBS and has a pH of about 7-7.8, suitable for storage and/or shipment at, for example, about 4° C. or lower. “Stability,” “stabilized,” and “stable” in the context of the present disclosure refers to the resistance of compositions of the disclosure and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

In certain embodiments, the pharmaceutical composition of the disclosure contain the biologically active agent(s) at a ratio of 0.01 to 25 mg/ml, 0.1 to 20 mg/ml, 0.2 to 18 mg/ml, 0.5 to 15 mg/ml, 0.7 to 12 mg/ml, 0.9 to 10 mg/ml, 1 to 8 mg/ml, 1.5 to 6 mg/ml, 2 to 5 mg/ml, 2.5 to 4 mg/ml, 0.5 to 3.0 mg/ml, 0.2 to 4.0 mg/ml, 0.4 to 2.0 mg/ml, and any numerical value or range within the range of 0.01 to 25 mg/ml.

Compositions and/or pharmaceutical compositions of the present disclosure of the disclosure may be administered to any patient or subject, including those patients or subjects that may benefit from a therapeutic effect provided by the delivery of a biologically active agent to one or more particular cells, tissues, organs, or systems or groups thereof. Although the descriptions provided herein of the compositions of the disclosure and pharmaceutical compositions including compositions of the disclosure are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including the composition of the disclosure may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### Methods of Delivering Therapeutic Agents to Cells and Organs

The present disclosure provides methods of delivering a biologically active agent to a cell or organ.

In some aspects, a composition of the disclosure may target a particular type or class of cells (e.g., cells of a particular organ or system thereof or cancerous cells). For example, a composition of the disclosure including a biologically active agent of interest may be specifically delivered to a liver, kidney, spleen, femur, or lung. Specific delivery to a particular class of cells, an organ, or a system or group thereof implies that a higher proportion of the composition of the disclosure including a biologically active agent are delivered to the destination (e.g., tissue) of interest relative to other destinations, e.g., upon administration of a composition of the disclosure to a mammal. In some aspects, specific delivery may result in a greater than 2 fold, 5 fold, 10 fold, 15 fold, or 20 fold increase in the amount of biologically active agent per 1 g of tissue of the targeted destination (e.g., tissue of interest, such as a liver) as compared to another destination (e.g., the spleen). In some aspects, the tissue of interest is selected from the group consisting of a liver, kidney, a lung, a spleen, a femur, an ocular tissue (e.g., via intraocular, subretinal, or intravitreal injection), vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a composition of the disclosure. An mRNA may additionally or instead be used to direct the synthesis and extracellular localization of lipids, carbohydrates, or other biological moieties. Alternatively, other biologically active agents or elements (e.g., lipids or ligands) of a the composition of the disclosure may be selected based on their affinity for particular receptors (e.g., low density lipoprotein receptors) such that the payload delivery system of the composition of the disclosure may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)₂ fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some aspects, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

In certain aspects, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a biologically active agent (e.g., an mRNA) in a given dose, where a dose of 1 mg/kg (mpk) provides 1 mg of a biologically active agent per 1 kg of subject body weight. In some aspects, a dose of about 0.001 mg/kg to about 10 mg/kg of a biologically active agent (e.g., mRNA) of the composition of the present disclosure may be administered. In other aspects, a dose of about 0.005 mg/kg to about 2.5 mg/kg of a biologically active agent may be administered. In certain aspects, a dose of about 0.1 mg/kg to about 1 mg/kg may be administered. In other aspects, a dose of about 0.05 mg/kg to about 0.25 mg/kg may be administered. A dose may be administered one time or multiple times in the same or a different amount, to obtain a desired level of sa-mRNA and mRNA expression and/or therapeutic, diagnostic, or prophylactic effect. In some aspects, a single dose may be administered, for example, prior to or after a surgical procedure or in the instance of an acute disease, disorder, or condition.

The composition of the disclosure may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more compositions of the disclosure may be administered in combination. Compositions can be administered concurrently with, prior to, or subsequent to, one or more other desired therapeutics or medical procedures. In general, each agent will be administered at a dose and/or on a time schedule determined for that agent. In some aspects, the present disclosure encompasses the delivery of compositions, or imaging, diagnostic, or prophylactic compositions thereof in combination with agents that improve their bioavailability, reduce and/or modify their metabolism, inhibit their excretion, and/or modify their distribution within the body.

It will further be appreciated that biologically active, or imaging active agents utilized in combination may be administered together in a single composition or administered separately in different compositions. In general, it is expected that agents utilized in combination will be utilized at levels that do not exceed the levels at which they are utilized individually. In some aspects, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

### Methods of Treating Diseases

The present disclosure provides methods of delivering a biologically active agent to a cell or organ. Compositions of the disclosure may be useful for treating a disease, disorder, or conditions characterized by dysfunctional or aberrant protein or polypeptide activity. Diseases, disorders, and/or conditions characterized by dysfunctional or aberrant protein or polypeptide activity for which a composition of the disclosure may be administered include, but are not limited to, rare diseases, infectious diseases (as both vaccines and therapeutics), cancer and proliferative diseases, genetic diseases (e.g., cystic fibrosis), autoimmune diseases, diabetes, neurodegenerative diseases, cardio- and reno-vascular diseases, lysosomal storage disorders and metabolic diseases. In particular, such compositions may be useful in treating cancer. The present disclosure provides a method for treating such diseases, disorders, and/or conditions in a subject by administering a composition of the disclosure wherein the payload induces apoptosis of cancer cells. In one aspect, the compositions of the present disclosure may be useful for treating adenocarcinoma, anal cancer, basal cell carcinoma, brain cancer, bladder cancer, bone cancer, breast cancer, cervical cancer, cholangiocarcinoma, colorectal cancer, endometrial cancer, esophageal cancer, gall bladder cancer, gastric cancer, glioblastoma, head and neck cancer, liver cancer, lung cancer, ovarian cancer, pancreatic cancer, prostate cancer, renal cancer, sarcoma, small bowel carcinoma, skin cancer, testicular cancer, uterine cancer, colon cancer, rectal cancer, intrahepatic bile duct cancer, thyroid cancer, eye cancer, squamous cell carcinoma and melanoma. In some aspects, the disorder is lymphoma, e.g. Epstein-Barr virus associated lymphoma, B-cell lymphoma, T-cell lymphoma Hodgkins and non-Hodgkins lymphoma. In some aspects, the cancer is a squamous cell cancer. In some aspects, the cancer is a head and neck squamous cell cancer. In some aspects, the cancer is a skin squamous cell carcinoma. In some aspects, the cancer is an esophageal squamous cell carcinoma. In some aspects, the cancer is a head and neck squamous cell carcinoma. In some aspects, the cancer is a lung squamous cell carcinoma. In some aspects, the compositions of the present disclosure may be useful for treating MC38 colon cancer, 4T1 breast cancer, Lewis lung carcinoma (LLC1), CT26 colon cancer, P53null KRasG12D pancreatic duct cancer or B16F10 melanoma.

A composition of the disclosure may be administered by any route. In some embodiments, compositions, including prophylactic, diagnostic, or imaging compositions including one or more nanoparticle compositions described herein, are administered by one or more of a variety of routes, including oral, intravenous, intramuscular, intra-arterial, intramedullary, intrathecal, intraparenchymal, subcutaneous, intraventricular, trans- or intradermal, interdermal, rectal, intravaginal, intraperitoneal, intraocular, subretinal, intravitreal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, intraocularly, subretinally, intravitreally, intraparenchymally or by any other parenteral route of administration or by inhalation. However, the present disclosure encompasses the delivery or administration of compositions described herein by any appropriate route taking into consideration likely advances in the sciences of drug delivery. In general, the most appropriate route of administration will depend upon a variety of factors including the nature of the composition including one or more biologically active agents (e.g., its stability in various bodily environments such as the bloodstream and gastrointestinal tract), the condition of the patient (e.g., whether the patient is able to tolerate particular routes of administration), etc.

In one aspect, the present disclosure provides a method of treating a subject having a tumor or cancer, comprising administering to a subject the pharmaceutical composition of the disclosure. In some aspects, the subject is a mouse, a rat, a rabbit, a cat, a dog, a horse, a donkey, a non-human primate, or a human. In one aspect, the pharmaceutical composition is administered intravenously, subcutaneously, intratumorally, intramuscularly, intratracheal, intraperitoneal, or intranasally.

In one aspect, the method comprises performing a first administering step, comprising administering the pharmaceutical composition of the disclosure wherein the at least one virus structural protein is a first viral capsid, a second envelope or a combination thereof, and performing a second administering step, comprising administering the pharmaceutical composition of the disclosure wherein the at least one virus structural protein is a second viral capsid that differs from the first viral capsid, a second viral envelope that differs from the first viral envelope or a combination thereof.

In one aspect, the method induces apoptosis of a cancerous cell or tumor, the method comprising contacting the cancerous cell with the pharmaceutical composition of the disclosure such that the first nucleic acid construct and at least one virus structural protein replicate within said cancerous cell, to express the GOI, wherein the first nucleic acid replication, expression of the GOI and the at least one virus structural protein replication within the cancerous cell results in cell death. In some aspects, the contacting is in vivo.

In one aspect, the present disclosure provides a method of delivering a GOI encoding a therapeutic gene product to cancerous cells in vivo comprising contacting the cancerous cells with the pharmaceutical composition of the disclosure.

In one aspect, the present disclosure provides a method of effecting in vivo synthesis of an oncolytic defective viral particle comprising contacting a cancerous cell with the pharmaceutical composition of the disclosure such that the first nucleic acid construct and the at least one virus structural protein replicate within said cancerous cell and express the GOI.

In one aspect, the present disclosure provides a method of inducing production of 1) a therapeutic payload 2) a pseudoviral particle and 3) sa-mRNA in a cell comprising contacting a cancerous cell with the pharmaceutical composition of the disclosure such that the first nucleic acid construct and the second nucleic construct produce the at least one virus structural protein, the sa-mRNA, and the GOI within said cancerous cell, and wherein the at least one virus structural protein form the pseudoviral particle, which encapsulate one or more of the sa-mRNA.

In one aspect, the present disclosure provides a method of increasing expression of a polypeptide encoded by a GOI in a cell comprising contacting the cell with the pharmaceutical composition of the disclosure such that the first nucleic acid construct and the second nucleic construct produce the at least one virus structural protein and the sa-mRNA within said cell, and the first nucleic acid construct expresses the GOI within said cell, wherein the at least one virus structural protein form a pseudoviral particle that encapsulate one or more of the sa-mRNA, and wherein expression of the polypeptide is increased by at least 100-fold compared to contacting the cell with the pharmaceutical composition in the absence of the second nucleic acid construct.

In one aspect, the present disclosure provides a method of increasing expression of a polypeptide encoded by a GOI in a cell comprising contacting the cell with the pharmaceutical composition of the disclosure such that the first nucleic acid construct and the second nucleic construct produce the at least one virus structural protein and the sa-mRNA within said cell, and the first nucleic acid construct expresses the GOI within said cell, wherein the at least one virus structural protein form a pseudoviral particle, which encapsulate one or more of the sa-mRNA, and wherein expression of the polypeptide is increased by 50-fold, 100-fold, 200-fold, 300-fold, 400-fold, 500-fold, or more compared to contacting the cell with the pharmaceutical composition in the absence of the second nucleic acid construct.

In one aspect, the present disclosure provides a method of increasing central memory CD8 T cell in a tumor draining lymph node or spleen comprising contacting a tumor with the pharmaceutical composition of the disclosure such that the first nucleic acid construct and the second nucleic construct produce the at least one virus structural protein and the sa-mRNA within said tumor to produce the at least one virus structural protein and the sa-mRNA, and the first nucleic acid construct expresses the GOI within said tumor, wherein the at least one virus structural protein form a pseudoviral particle, which encapsulate one or more of the sa-mRNA, and wherein central memory CD8 T cell count is increased by 5-fold, 6-fold, 7-fold, 8-fold, 9-fold, 10-fold, or more compared to contacting the cell with the pharmaceutical composition in the absence of the second nucleic acid construct.

In one aspect, the present disclosure provides a method of treating cancer, comprising administering to a subject a composition comprising an IL-12 and an IL-18. In some aspects, the IL-12 is a wildtype human IL-12. In some aspects, the IL-12 is a variant IL-12 having at least 90% amino acid sequence identity to a wildtype human IL-12. In some aspects, the IL-12 is a wildtype human IL-18. In some aspects, the IL-18 is a variant IL-18 having at least 90% amino acid sequence identity to a wildtype human IL-18. In some embodiments, the variant IL-18 is encoded by SEQ ID NO. 101.

In one aspect, the present disclosure provides a method of treating a subject with a payload delivery system encapsulating a mRNA encoding at least one gene of interest (GOI) or a plurality of GOIs and minimizing immunogenicity of the payload delivery system comprising administering the pharmaceutical composition of the disclosure to the subject such that the first nucleic acid construct and and the second nucleic construct produce the at least one virus structural protein and the sa-mRNA within a cell of the subject, and the first nucleic acid construct expresses the GOI within said cell, wherein the at least one virus structural protein form a pseudoviral particle, which encapsulate one or more of the sa-mRNA, and wherein said non-viral payload delivery system has reduced immunogenicity compared to viral payload delivery systems.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein by reference, are useful and recognized reference textbooks of organic synthesis known to those in the art. The following descriptions of synthetic methods are designed to illustrate, but not to limit, general procedures for the preparation of compounds of the present disclosure.

### EXAMPLES

While the standard viral genome encodes all the viral proteins required for viral replication, some viral variants contain mutations that result from either small mutations or drastic modifications of the viral genome, which may render the virus unable to complete a full replication cycle, called defective viral genome (DVG) (Vignuzzi, 2019, Nat Microbiol 4: 1075-1087). DVGs play complicated roles on interactions between the host cell and the virus (e.g. Rezelj, et al. 2021, Nat Commun 12: 2290). The oncolytic defective virus used in the experiments described in the following sections was a VEE (TC-83 strain) with a DVG engineered using with the structural proteins deleted for use as a cancer immunotherapy.

The sa-mRNA used in the experiments described in the following sections was derived from the viral genome of Venezuelan equine encephalitis virus (TC-83) and was engineered by replacing the subgenome that encodes structural proteins for capsids and envelopes with therapeutic payloads, as described in PCT/US2023/066903, which is fully incorporated herein by reference.

Typically, the therapeutic oncolytic defective virus is engineered in vitro, which is very time consuming and expensive (see, e.g., Ungerechts, et al., 2016, Mol. Ther. Methods Clin. Dev. 3: 16018). In one aspect, the present invention is a method of producing pseudoviral particles encapsulating sa-mRNA encoding a therapeutic GOI, such as IL12, and a therapeutic in vivo using the composition of the disclosure, which comprises a payload delivery system encapsulating a first nucleic acid construct encoding a sa-mRNA encoding the therapeutic and a second nucleic acid construct encoding an mRNA encoding at least one virus structural protein.

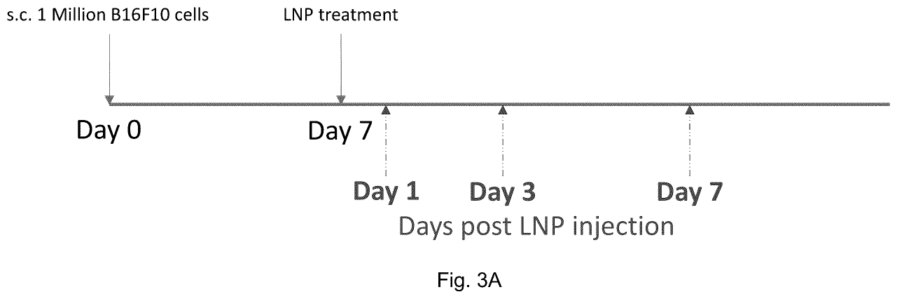

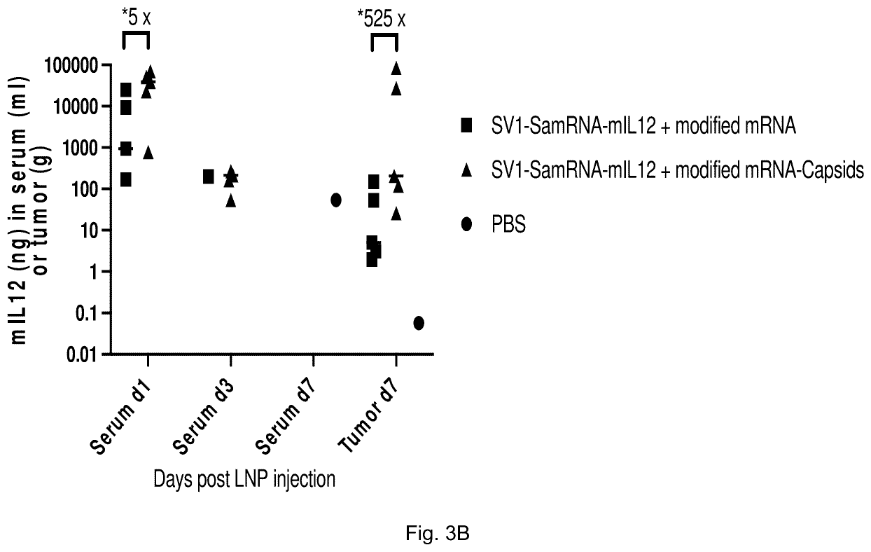

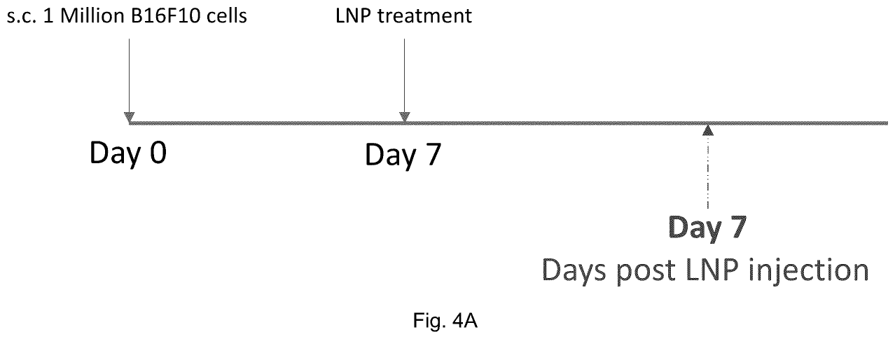

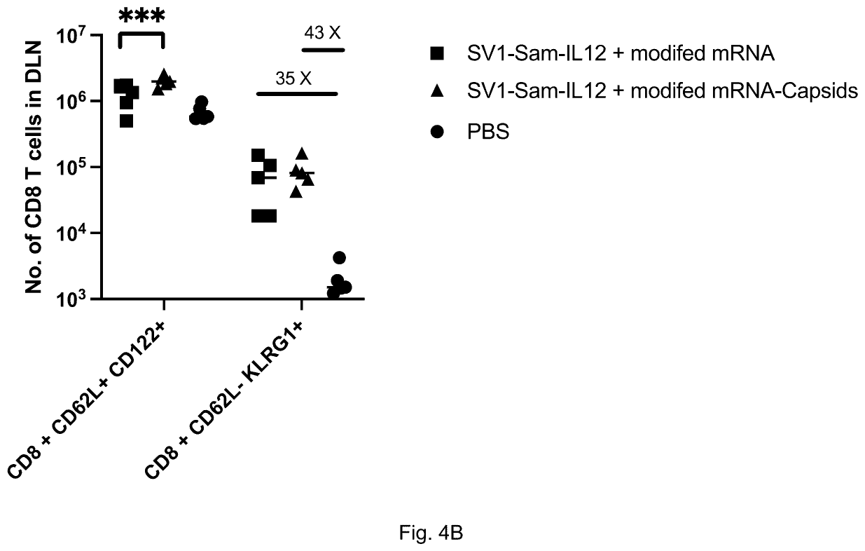

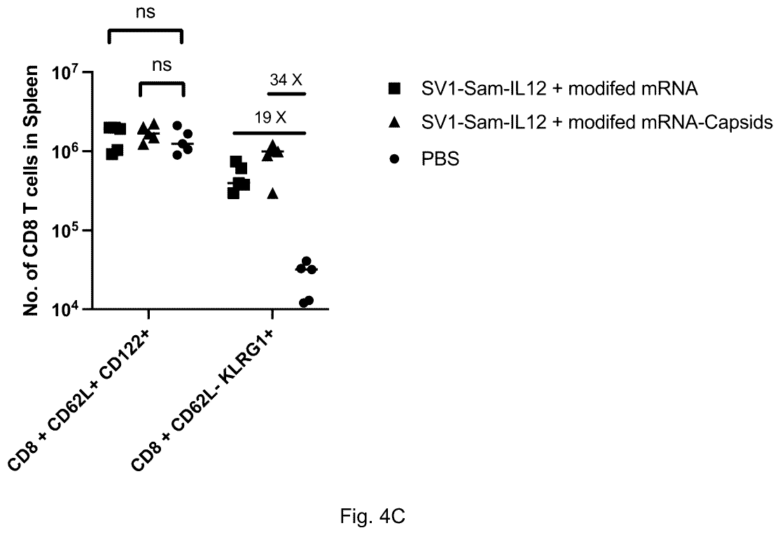

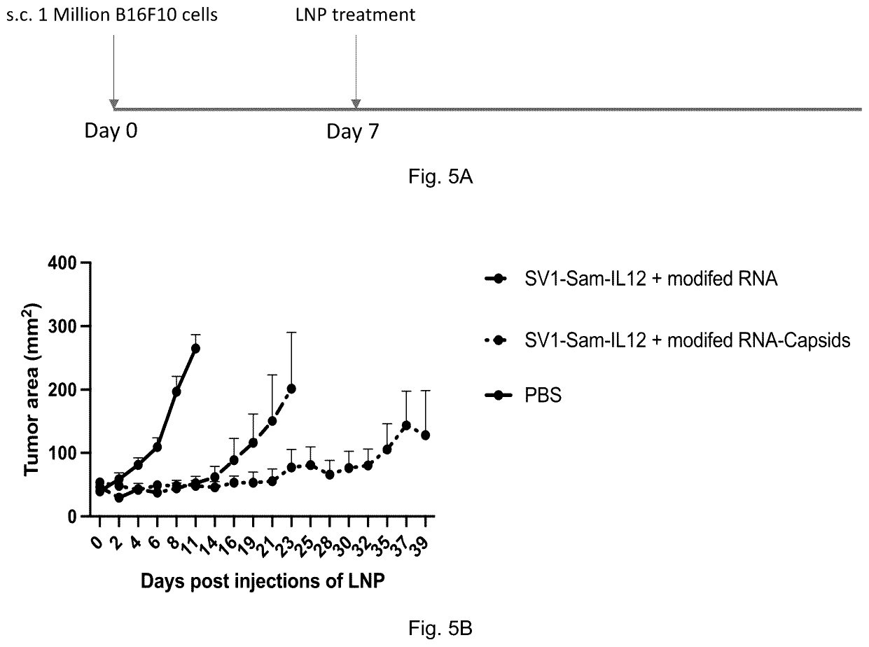

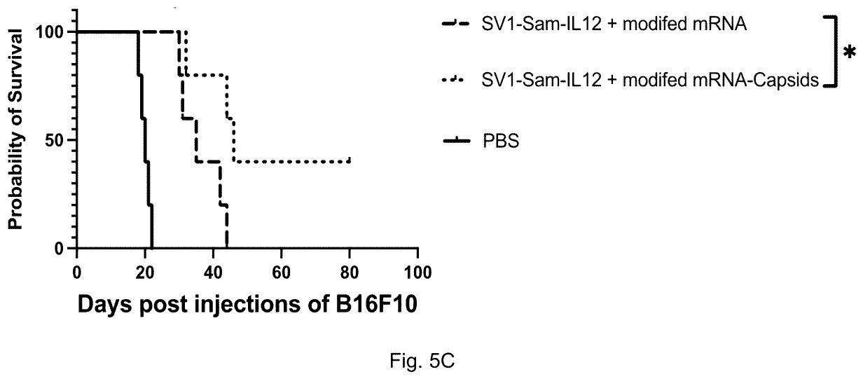

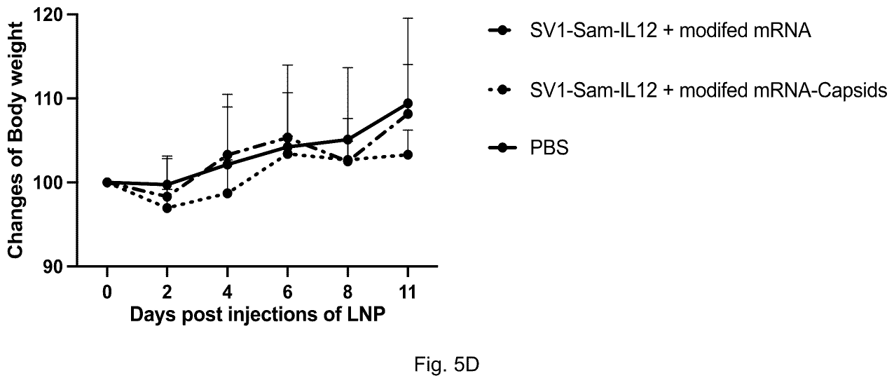

Notably, the composition used in the studies below, composed of LNPs encapsulating sa-mRNA encoding IL12 (SamRNA-IL12; SEQ ID NO: 41) in combination with a modified mRNA encoding the structural proteins from TC-83 strain of VEE (SEQ ID NO: 26), showed a over 500× expression of IL12 in the intratumorally injection site compared to mice treated with a negative control (FIG. 3B). Furthermore, the composition of the disclosure dramatically enhanced central memory CD8 T cell presence in the tumor draining lymph node, showing 10× higher numbers of effective CD8 T cells in both tumor-draining lymph node (TDLN) and spleen (FIGS. 4B-4C). Consequently, tumor growth was significantly suppressed in mice treated with the composition of the disclosure compared to the negative control (FIG. 5B), with 40-60% of the treated mice being tumor free after a single treatment with the composition of the disclosure. Importantly, the treatment showed reduced side effects such as body weight changes in the treated mice compared to mice treated with the negative control.

### Example 1: In Vitro Testing

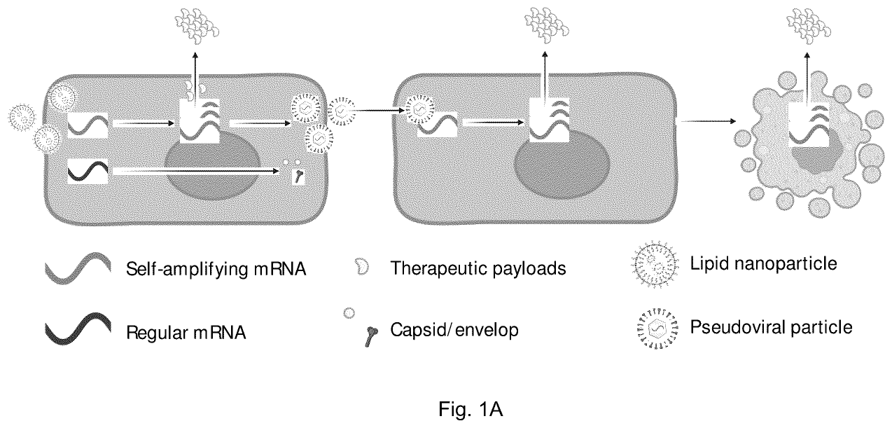

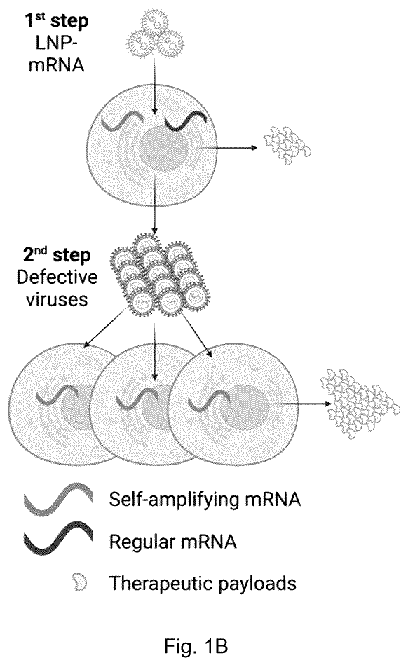

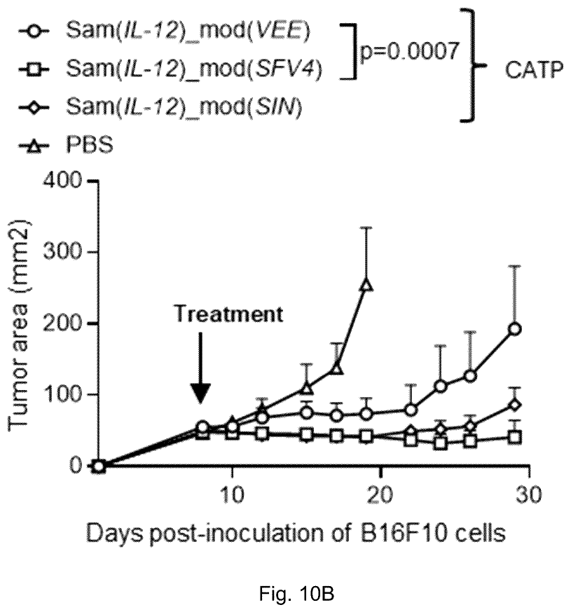

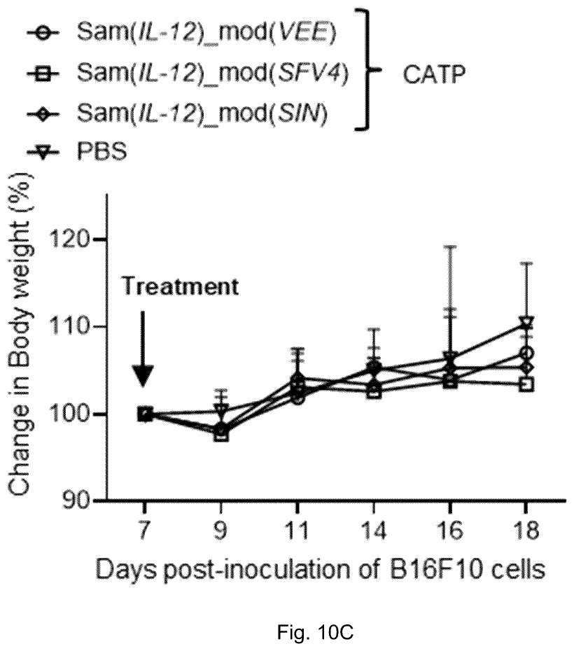

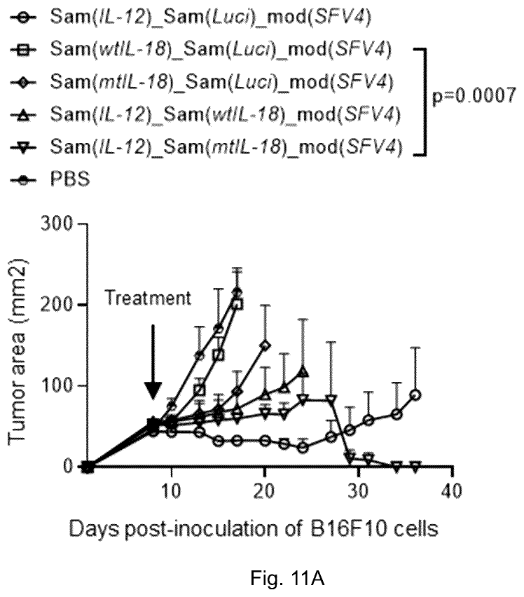

In one aspect, after the composition of the present disclosure is introduced to a host cell, the payload of the composition in the host cell will be expressed, and the expression of the payload of the composition will cause cell death of the host cell and adjacent cells as shown in FIG. 1. On the left side of FIG. 1, a LNP delivery system encapsulating a sa-mRNA encoding a therapeutic GOI and a mRNA encoding the capsid and envelope proteins of an oncolytic virus is introduced into the host cell. The payloads are released from the LNP inside the host cell and produces a therapeutic, new copies of the sa-mRNA and the capsid and envelope proteins of an oncolytic virus. The capsid and envelope proteins of an oncolytic virus form a pseudoviral particle encapsulating the new copies of the sa-mRNA, which is released to infect adjacent cells. The host cell will release the therapeutic out of the cell, which will affect adjacent cells. In the middle of the diagram, an adjacent cell is infected with the pseudoviral particle encapsulating the copies of the sa-mRNA and produces the therapeutic. On the right side of the diagram, the oncolytic effects of the pseudoviral particle and increased adaptive immune responses triggered by the therapeutic cause immunogenic cell death in affected cells, including the host cell and the adjacent cell. By co-delivering SamRNA from VEE encoding therapeutic payloads and modified mRNA encoding optimized viral capsids/envelop using lipid nanoparticles, the inherent amplification of self-amplifying mRNA is leveraged, which is referred to as cascade amplification of therapeutic payloads (CATP) in this work. Additionally, as shown in FIG. 10A, the production of defective viruses enables the infections of adjacent cells, further amplifying the therapeutic payloads and enhancing overall efficacy.

As shown in FIG. 10A, HEK293 cells were treated by LNP encapsulated SamRNA mRNA encoding with eGFP and modified mRNA encoding with envelop and capsids from VEE or LNP encapsulated SamRNA mRNA encoding with eGFP and modified mRNA encoding firefly Luciferse. The GFP percentages were then determined by flow cytometer at day 1, 2, 3, 4 post transfection.

VEE SamRNA plasmid DNA was prepared based on the constructs previously developed in the PCT/US2023/066903, “Synthetic SamRNA Molecules with Secretion Antigen and Immunomodulator”. Modified mRNA plasmid DNA was prepared based on the constructs previously developed in the PCT/US2023/085919, “Compositions and Methods for Delivering Molecules” the contents of each of which are herein incorporated by reference.

eGFP, firefly luciferase, mouse IL-12, and mouse IL-18 were cloned into XbaI and ClaI after the subgenomic promoter of VEE SamRNA plasmid DNA. Capsids/envelops from VEE, Sindbis virus, and Semliki Forest virus (SFV) 4 were cloned after T7 promoter using seamless cloning technology.

SamRNA were in vitro transcribed (IVT) from the templates of linearized VEE DNA constructs using the NEB RNA synthesize kit (Catalog No. E2050S). The synthesized mRNAs were then capped and methylated by Cellscript kits (Catalog No. C-SCCS1710). Modified mRNAs were in vitro transcribed (IVT) from the templates of linearized VEE DNA constructs using the NEB RNA synthesize kit (Catalog No. E2040S) plus pseudouridine (Catalog No. N-1019) and Cleancap (Catalog No. N-7413) from Trilink. The quantity and purity of SamRNA and modified mRNA were assessed by Nanodrop and gel electrophoresis.

The size, polydispersity index and zeta potentials of LNPs were measured using dynamic light scattering (Z-100-Z2(MTS), HORIBA, Ltd.). Diameters are reported as the intensity mean peak average. To calculate the nucleic acid encapsulation efficiency, a modified Quant-iT RiboGreen RNA assay (Invitrogen) was used. Results of encapsulation efficiency can be found in Table A below.

TABLE A
Encapsulation efficiency and particle properties of LNPs

| | LNP-Sam | LNP-Sam | LNP-Sam | LNP-Sam |
|---|---|---|---|---|
| | (IL12)_{—} | (IL12)_{—} | (IL12)_{—} | (IL12)_{—} |
| | mod(Lucif) | mod(VEE) | mod(SFV4) | mod(SIM) |
| Size (nm) | 119.3 ± 0.6 | 123.2 ± 2.1 | 122.2 ± 4.0 | 120.0 ± 1.8 |
| PDI | 0.17 ± 0.02 | 0.17 ± 0.04 | 0.17 ± 0.02 | 0.21 ± 0.01 |
| Zeta Potential | −3.2 ± 3.6 | −6.8 ± 1.9 | −7.1 ± 4.3 | −4.3 ± 1.0 |
| EE(%) | 90.5 ± 1.4 | 93.4 ± 0.6 | 93.5 ± 1.1 | 92.8 ± 1.5 |

For in vitro transfection, the cells were plated at 30% confluence at day 0. The formulated nanoparticles containing 100 ng mRNA were added to 100 ul of medium in a well of a 96-well plate at day 1. Then the transfected cells were followed with analysis accordingly.

Cell lines HEK293 (ATCC CRL-1573), B16F10 (ATCC CRL-6475), CT26 (ATCC CRL-2638), were cultured following vendor instructions. MC38 and KPC cells were maintained in the Li lab at the University of Michigan. Female C57BL/6 (Charles River Lab), Balb/C (Charles River Lab) mice at 6-8 weeks of age were purchased and maintained in the animal facility at the Mispro Biotech Service Corporation, Alewife. All animal studies and procedures were carried out following federal, state and local guidelines under an institutional animal care and use committee-approved animal protocol by the Committee of Animal Care at Mispro.

One million B16F10, MC38, CT26 or KPC cells in 50 μl of sterile PBS were s.c. injected into the flank of mice. At 7 days later when tumors reached ˜50 mm² in size, animals were injected intratumorally with PBS (control) or LNPs in 50 μl of PBS as indicated. For the rechallenge, 0.1 million B16F10 or 0.2 million MC38 cancer cells were s.c. injected into another side flank of tumor free mice or naïve mice.

Antibodies against mouse CD4 (Catalog No. 100412), CD8 (Catalog No. 100766), CD3e (Catalog No. 155612), CD62L (Catalog No. 104406), CD122 (Catalog No. 123216), KLRG1 (Catalog No. 138418), 7-AAD (Catalog No. 420404), and Zombie Aqua (Catalog No. 423102) were bought from Biolegend. All the antibodies were diluted 1:50. The live/dead dye Zombie Aqua was diluted 1:300. The single-cell suspensions were filtered by 70-μm nylon strainers and stained as described 20. Stained samples were analyzed using a Symphony A5 FACS analyzer from BD Biosciences. All flow cytometry data were analyzed using FlowJo software (Flowjo LLC).

Tumors were collected and ground in tissue protein extraction reagent (T-PER™, Thermo Fisher Scientific, cat. no. 78510) in the presence of 1% proteinase and phosphatase inhibitors (Thermo Fisher Scientific, cat. no. 78442). The lysates were incubated at 4° C. for 30 min with slow rotation then centrifuged to remove debris. The supernatants were transferred to a clean tube for ELISA or Luminex analysis. Mouse IL-12 in tumor tissue supernatants or in serum were measured by ELISA kits from R&D (Catalog No. DY419) following the manufacturer's instructions.

Data were statistically analyzed by one-way or two-way ANOVA or by Student's t-test using GraphPad PRISM as indicated. Animals were randomized to treatment groups once the mean tumor size around 50 mm2 was reached by the tumor-inoculated cohort. No data were excluded from the analyses. The investigators were not blinded to allocation during experiments and outcome assessments. The samples sizes for in vitro analysis were three (triplicates) and for in vivo analysis are as annotated in figure legends. The details of statistical analysis for figures and Extended Data Figures are included in the Source Data files.

### Example 2: In Vitro Testing

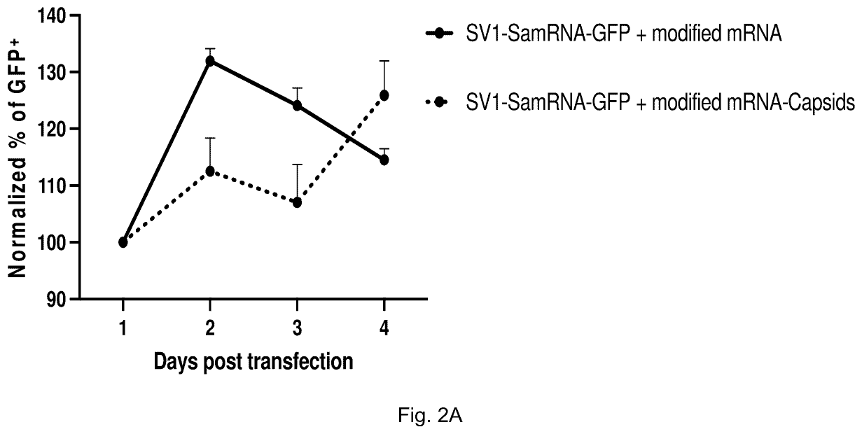

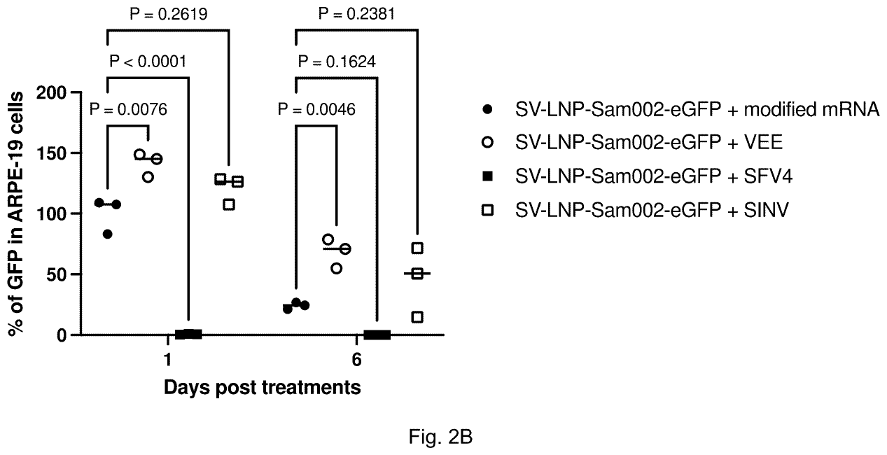

To study the effects of delivering the composition of the disclosure to a host cell and production of the sa-mRNA, GOI and pseudoviral particle by the host cell in vitro. Sa-mRNA encoding GFP (SamRNA-GFP; SEQ ID NO: 42) and modified mRNA encoding luciferase (mRNA-Luc) (SEQ ID NO: 43) or modified mRNA encoding Cap/Env were encapsulated in an LNP comprising lipid components SV1 and P6 and transfected into HEK293 cells. The SamRNA-GFP with modified mRNA encoding Cap/Env showed a more gradual increase in GFP expression compared to mRNA-Luc post transfection, likely due to higher immunogenicity of Cap/Env compared to Luc, which triggered innate immunity and resulted in feedback inhibitions. Interestingly, as shown in FIG. 2A, while GFP expression showed a more gradual increase in days 2 to 3, GFP expression increased at day 4.

To validate the platform in vitro, HEK293 cells were co-transfected with SamRNA encoding eGFP and modified mRNA encoding VEE capsids/envelop (CATP: Sam(eGFP)_mod(VEE)) and were compared to cells transfected with SamRNA encoding eGFP (Sam(eGFP)_mod(Luci). Transfection efficiency was determined by flow cytometry and the percentages of GFP cells were normalized by transfection efficiencies in the group of encapsulated self-amplifying mRNA (Sam002) encoding eGFP and modified mRNA encoding luciferase at day 1 post treatments. Statistic significance was analyzed by two-way ANNOVA. As anticipated, the percentages of eGFP-positive cells were initially lower in CATP group than control group within the first three days post transfection. However, the CATP group markedly expanded on day 3 and surpassed the control group after day 4, while the control group started to decay after day 2, as shown in FIG. 2A. This observation underscored the key feature of CATP in terms of amplifying reporter genes in vitro.

Having validated this platform in HEK293 cells, a similar experiment was conducted in ARPE-19 cells (human retinal pigment epithelial cells). APRE-19 cells were co-transfected with SamRNA (Sam002) encoding eGFP and modified mRNA encoding: VEE capsids/envelop (CATP: Sam(eGFP)_mod(VEE)); SFV4 capsids/envelop (CATP: Sam(eGFP)_mod(SFV4)); or SINV capsids/envelop (CATP: Sam(eGFP)_mod(SINV)). The capsid/envelope-transfected APRE-19 were compared to cells transfected with Sam002 RNA encoding eGFP (Sam(eGFP)_and modified RNA encoding luciferase (mod(Luci). The results are shown in FIG. 2B.

P6 ionizable lipid was designed and synthesized in the patent (PCT/US2023/017777). An ethanol phase was prepared by dissolving P6, (DOPE (870341, Avanti Research), cholesterol (7001, Avanti Research), and DMG-PEG2000 (880151, Avanti Research) at a predetermined molar ratio of 30:15:50:1.5. The aqueous phase was prepared in 50 mM citrate buffer (pH 4.5, AAJ60024AK, Fisher Scientific) containing SamRNA and/or mRNA. All mRNA samples were stored at −80° C. and thawed on ice prior to use. The aqueous and ethanol phases were combined at a 3:1 ratio with a lipid-to-RNA (N/P) ratio of 4.2, using a microfluidic chip device (INano™ L system, Micro & Nano) at a flow rate of 12 mL/min. The resulting lipid nanoparticles (LNPs) were dialyzed against 1×PBS (MT21040CMX, Fisher Scientific) using a Slide-A-Lyzer™ MINI Dialysis Devices, 20K MWCO (Catalog No. 88405 Fisher Scientific) at 25° C. for 80 min and stored at 4° C. prior to injection.

### Example 3: In Vivo Testing

To study the effects of delivering the composition of the disclosure to a host cell and production of the sa-mRNA, GOI and pseudoviral particle by the host cell in vivo. The amplification capacity of CATP was performed in a highly immune resistant mouse B16F10 melanoma model via a single intratumoral injection of lipid nanoparticles encapsulating CATP encoding mouse cytokine IL-12. Delivery of recombinant IL-12 via different technologies has been shown to exhibit potent anti-tumor immunity by stimulating CD4, CD8 T cells, NK cells, and B cells, while its prolonged systemic exposure is associated with host toxicity. As shown in FIG. 3A, sa-mRNA encoding mIL12 and mRNA-Luc or modified mRNA encoding Cap/Env were encapsulated in an LNP comprising lipid components SV1 and P6 and transfected into C57B6L mice seven days after the mice were inoculated with 1 million B16F10 melanoma cells. Seven days post-inoculation, mice were intratumorally treated with PBS, or LNP encapsulating 10 g SamRNA encoding with mouse IL-12 (mIL-12) and 1 g modified mRNA encoding with firefly Luciferase or LNP encapsulated 10 g of SamRNA encoding mIL-12 and 1 g modified mRNA encoding capsids/envelops from VEE, SFV4 and SIN.

Serum was collected at day 1, 3, and 7 post injection, and tumor tissues were collected at day 7 post injection. At day 1 post injection, the average level of mouse IL12 within each group of 5 replicates was 5× higher in the mice treated with the composition including SamRNA-mIL12 and mRNA encoding Cap/Env compared to the composition including SamRNA-mIL12 and mRNA-Luc. At day 3 and 7 post injection, the amount of mIL12 in the serum showed a significant decrease in all three groups. Surprisingly, the level of mIL12 in the tumor of the mice treated with SamRNA-IL12 and modified mRNA encoding Cap/Env is 500× higher than the mice treated with SamRNA-IL12 and mRNA-Luc. Compared to the control group containing SamRNA encoding mouse IL-12 (Contr: Sam(IL-12)_mod(Luci)), the CATP group containing SamRNA-IL12 and VEE capsids/envelop (CATP: Sam(IL-12)_mod(VEE)) displayed five-fold increase in mouse serum IL-12 levels on day 1, remained detectable on day 3, and became undetectable by day 7. Notably, the mouse IL-12 levels in tumor sites increased by 525 times in CATP group relative to that of control group, as shown in FIGS. 3A-3B. These results demonstrated that the CATP system outperformed the conventional SamRNA platforms in the expression of transgenes both in vitro and in vivo. To further evaluate whether the increased therapeutic levels achieved through a single dose of CATP-mIL-12 improves therapeutic efficacies in the B16F10 melanoma model that's a poorly immunogenic and highly aggressive model for cancer immunotherapy. On day 7 post-inoculation of cancer cells when tumor area reached approximately 50 mm, mice were intratumorally treated with a single dose of CATP or corresponding controls as described in FIG. 3A. Consistent with the increased therapeutic levels observed in FIG. 3B, tumors in the CATP-IL-12 group showed improved regression, as shown in FIG. 5B, resulting in 40% of tumor-free mice (p=0.0357, as shown in FIG. 5C). Notably, body weight changes remained below 4%, as shown in FIG. 5D, aligning with the undetectable serum IL-12 levels on day 7 post injections of LNP-mRNA, as shown in FIG. 3B. In summary, the CATP not only increases the expression of therapeutic payloads but also enhances therapeutic efficacy while maintaining the same dosage and safety profiles, demonstrating its potential as a safe and effective cancer immunotherapy strategy.

### Example 4: In Vivo Immunogenicity Testing

To better understand the immune responses of the in vivo testing of Example 3, three groups of five C57BL6 mice were subcutaneously inoculated 1 million B16F10 cells. Seven days later, the tumors, which were approximately 50 mm² in size, were intratumorally treated according to the treatment plan described in Example 3 and shown in FIG. 4A. Then the spleen (FIG. 4C) and the TDLN (FIG. 4B), here the inguinal lymph node, were isolated and prepared into single cell suspensions for fluorophore activated cell sorting. The live CD8 T cells were gated and further analyzed by central markers (e.g. (CD8+ CD62L+ CD122+) CD62L and CD122, or by effective makers (CD8+ CD62L−KLRG1+) CD62L and KLRG1 (e.g. Liu, et al., 2015, Front Immunol 6:494; Herndler-Brandstetter, et al., 2018, Immunity 48:716-729 e718). In contrast to the tumors treated with PBS, both groups treated with a composition including SamRNA-mIL12 showed 10× higher effector CD8 T cell levels. Interestingly, the central memory CD8 T levels were significantly higher in the group treated with the composition including SamRNA-IL12 and mRNA encoding Cap/Env than the group treated with the composition including SamRNA-IL12 and mRNA-Luc.

### Example 5: In Vivo Therapeutic Efficacy Testing

To better understand the therapeutic efficacy of the in vivo testing of Example 3, three groups of five C57BL6 mice were subcutaneously inoculated 1 million B16F10 cells. Seven days later, the tumors, which were approximately 50 mm in size, were intratumorally treated according to the treatment plan described in Example 3 and shown in FIG. 5A. Then the tumor size, survival rate, and body weight were measured, as shown in FIGS. 5B-5D respectively. Tumor growth was significantly delayed in the group treated with the composition including SamRNA-mIL12 and mRNA encoding Cap/Env. 2 of the 5 mice were tumor free in the group treated with the composition including SamRNA-mIL12 and mRNA encoding Cap/Env, which is consistent with the increase of mIL12 and central memory CD8 T cells in vivo seen in Example 3 and 4 (FIGS. 3B and 4B). To determine side effects of the treatment, the body weight of the tumor bearing mice were measured and showed only a slight, less than 5%, decrease 2 days post injection, which gradually recovered to pre treatment levels after day 2.

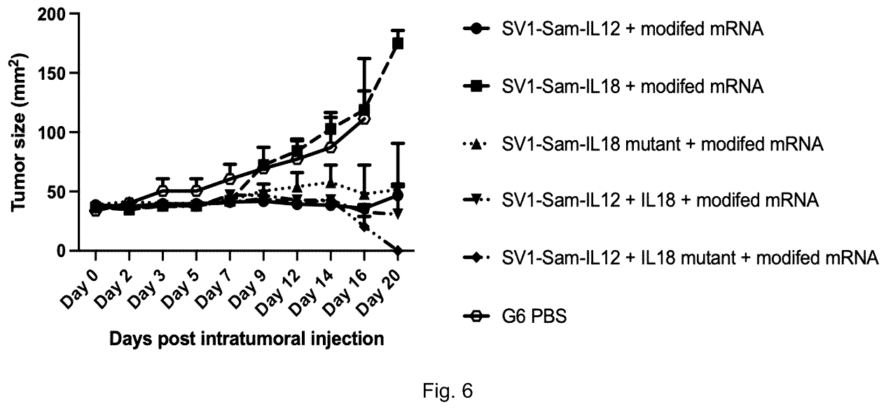

To understand the therapeutic efficacy of the composition of the disclosure on a different cancer type, a similar study was conducted using MC38 colon cancer cells in vivo. Six to eight weeks old C57B6L mice (5 replicates in each group) were subcutaneously inoculated with 0.5 million MC38 colon cancer cells. Seven days post inoculation, the mice were intratumorally injected with PBS as a negative control; an LNP comprising lipid components SV1 and P6 encapsulating SamRNA-mIL12 and a modified mRNA encoding capsid and envelop proteins from VEE virus; an LNP comprising lipid components SV1 and P6 encapsulating a sa-mRNA encoding GOI mIL18 (SEQ ID NO: 53) and a modified mRNA encoding capsid and envelop proteins from VEE virus; or an LNP comprising lipid components SV1 and P6 encapsulating a sa-mRNA encoding mIL18 mutant (SEQ ID NO: 54) and a modified mRNA encoding capsid and envelop proteins from VEE virus. FIG. 6 shows tumor area of the MC38 colon cancer tumor (Y-Axis) versus days post intratumorally injection (X-Axis) in the treated mice. The statistical analysis were performed by 2-way ANNOVA.

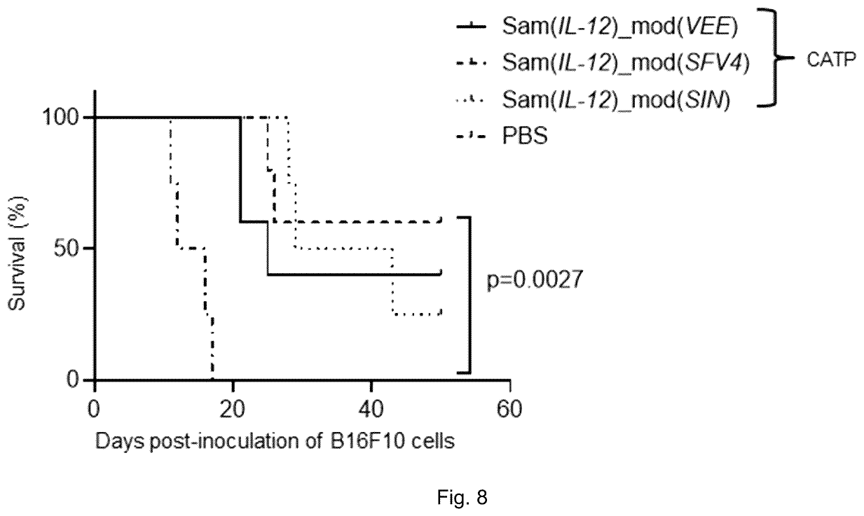

To investigate the roles of different capsids/envelops in determining the amplification efficiency for CATP, an evaluation of the capsids/envelops derived from three commonly used alphaviruses, including VEE, Sindbis virus (SIN), and Semliki Forest virus (SFV4) was conducted. Interestingly, the SFV4-derived capsids/envelops exhibited the strongest regression of tumor growth, as shown in FIG. 10B, resulting in a 60% tumor free rate with minimal weight changes, as shown in FIG. 8 and FIG. 10C. These findings suggest that CATP leveraging SFV4 capsids/envelops may generate oncolytic virus-like particles, in coincidence with other researchers' observations of oncolytic effects associated with SFV4 viruses.

To further enhance therapeutic efficacy, the therapeutic payloads were optimized by combining mouse IL-12 with mouse IL-18, as IL-18 enhances polarizations towards Type 1 inflammation in the presence of IL-12. However, IL-18 binding protein (IL-18BP) has been reported to be upregulated in solid tumors thereby inhibiting IL-18 activity. Therefore, wildtype mouse IL-18 was compared to a mutant form, which was designed to disrupt interactions with IL-18BP.

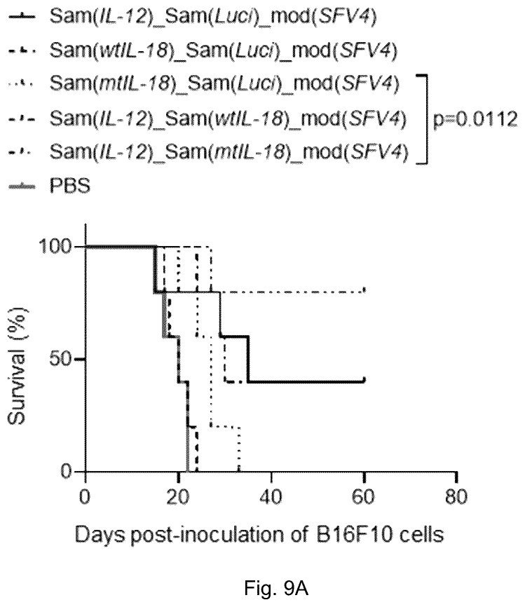

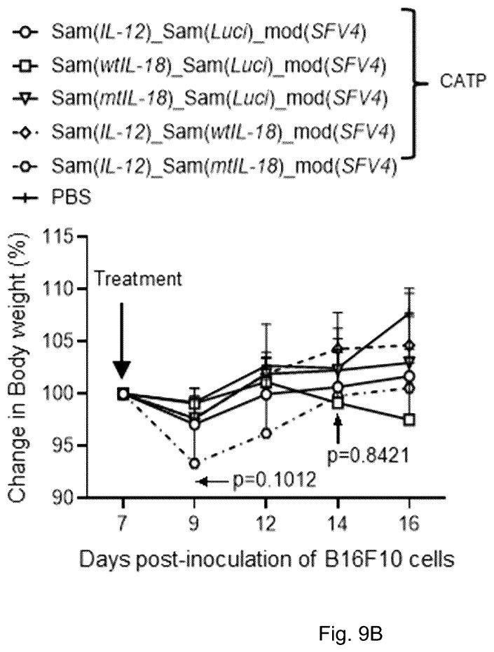

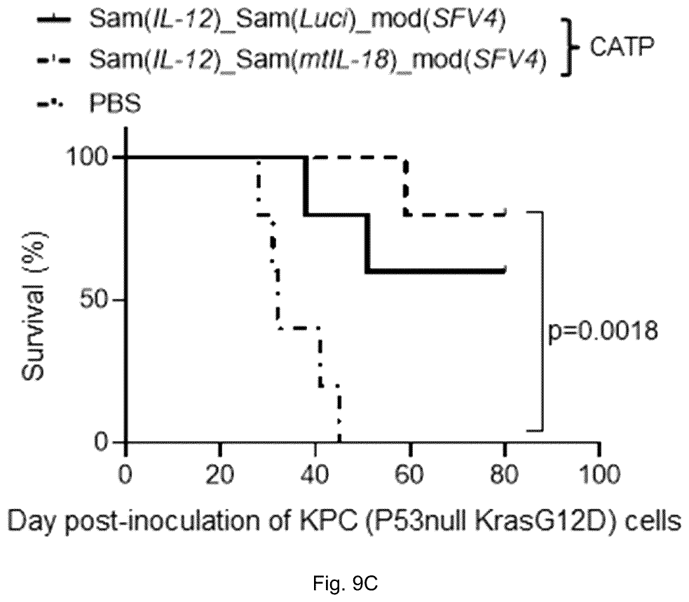

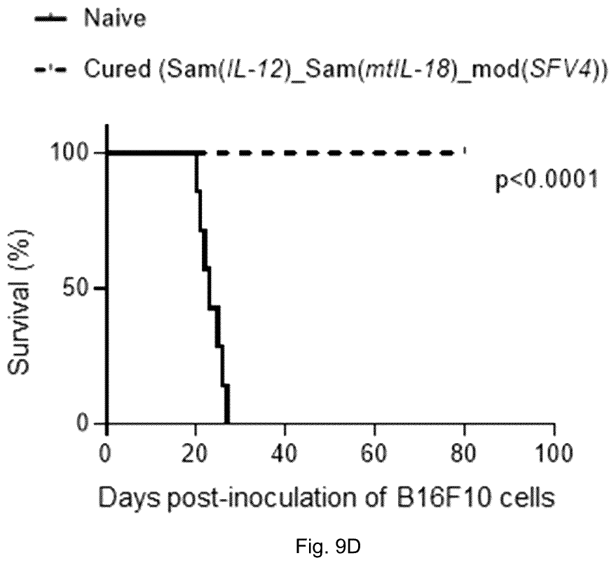

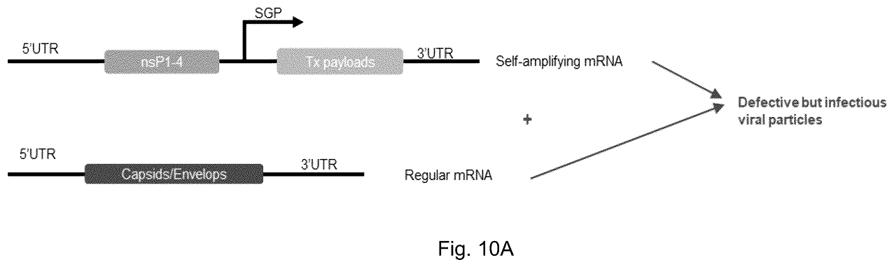

Following a single dosing in tumors with a starting size of −50 mm, the combination of mIL-12 with mutant mIL-18 in the CATP system significantly inhibited the tumor growth and increased the tumor-free rates to 80% in the B16F10 melanoma model. This outcome markedly surpassed the 40% tumor-free rates achieved by mIL-12 plus wild-type mIL-18, or mIL-12 alone, as shown in FIG. 6 and and FIG. 9A. While the CATP with mouse mutant IL-18 alone effectively inhibited tumor growth, it failed to cure any tumor-bearing mouse, as shown in FIG. 6 and FIG. 9A, indicating that the anti-tumor effects of mutant IL-18 were dependent on mouse IL-12. Although the combination of mIL-12 with mutant mIL-18 was initially associated with body weight loss of >6% after CATP administration, they returned to normal body weights similar to the control group (PBS) one week after the dosing (14 days post tumor inoculation, p=0.8421), as shown in FIG. 9B. These findings suggest that the CATP system combining mIL-12 and mutant IL-18 displayed superior efficacy without compromising the safety following a single administration in tumors.

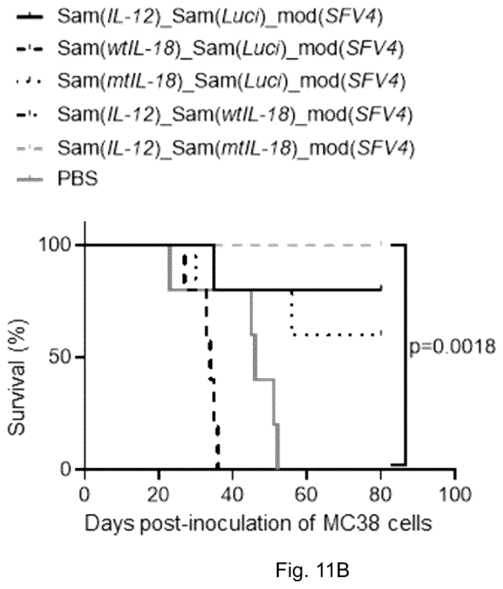

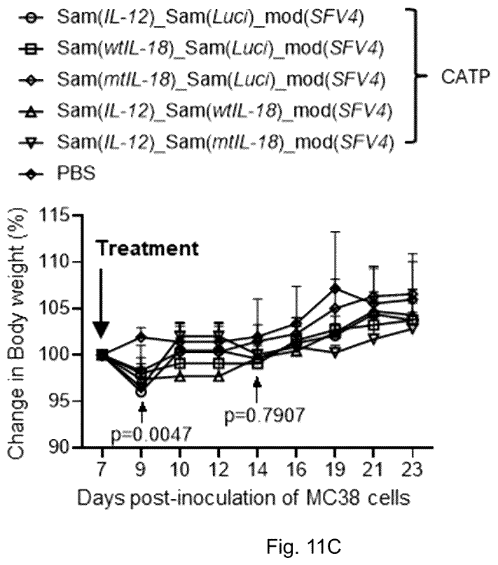

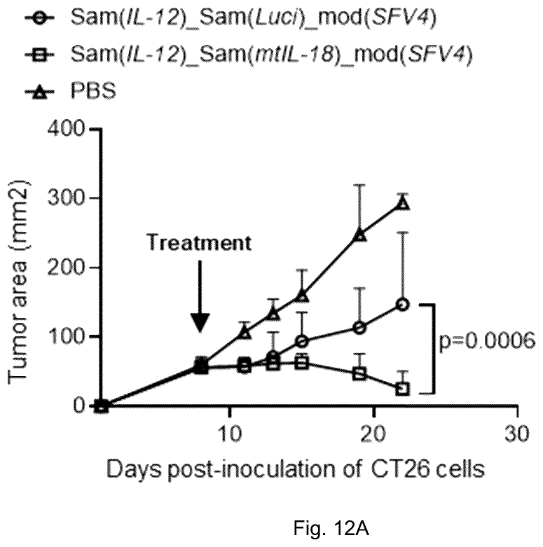

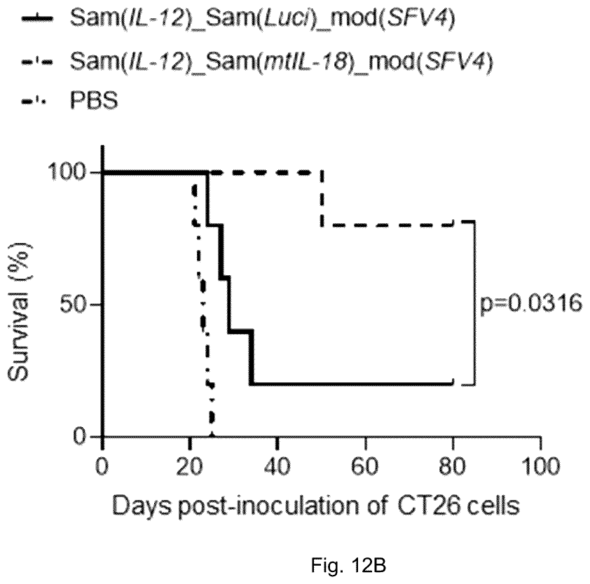

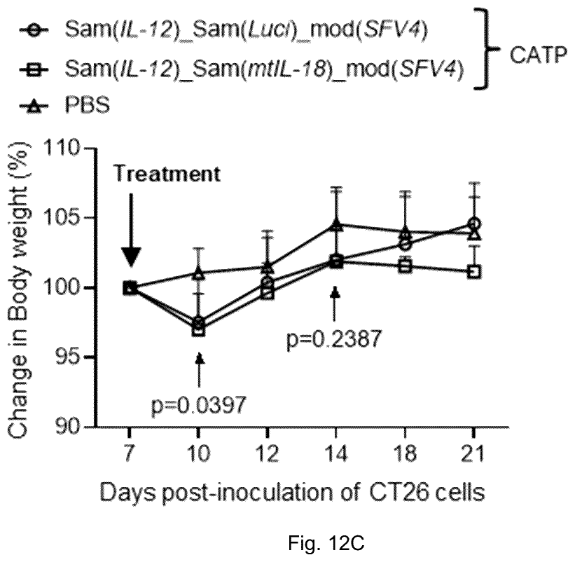

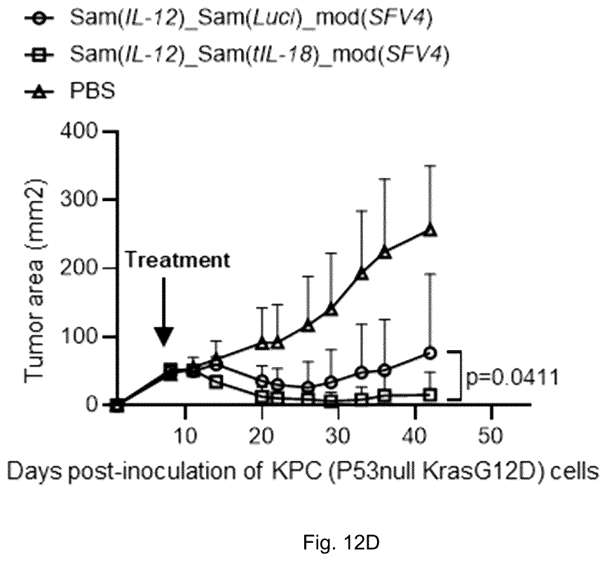

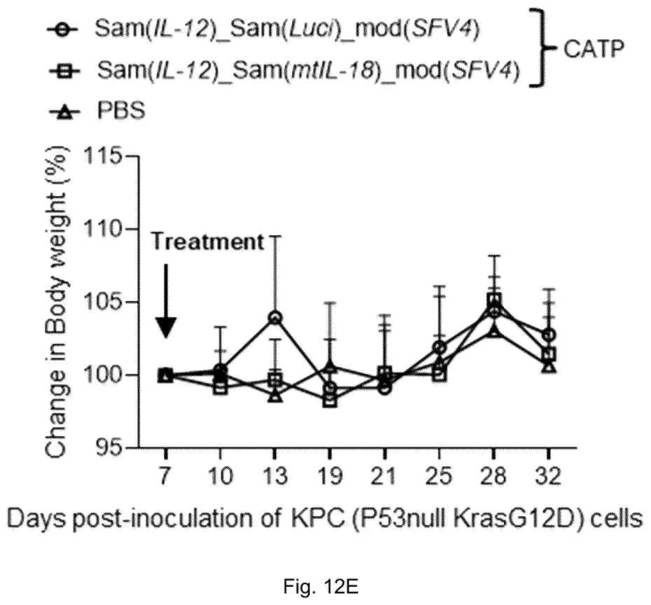

To generalize the CATP system to other syngeneic mouse cancer models, the MC38 colorectal cancer model was evaluated, where the CATP therapy combing mIL-12 and mutant mIL-18 eradicated all the tumors, as shown in FIGS. 11A-11C. In the CT26 colon cancer model, the CATP therapy with mIL-12 and mutant mIL18 achieved 80% tumor-free rates, outperforming 20% tumor free rates with CATP mIL-12 alone. There is no significant difference between the body weights of CATP therapy combing mIL-12 and mutant mIL-18 group and the control group (PBS) one week after the dosing, as shown in FIGS. 12A-12C. To assess the efficacy of this approach in genetically driven cancers, mice were subcutaneously inoculated with P3^{null} Kras^{G12D} pancreatic duct cancer¹⁶ cells and treated intratumorally. Consistent with previous findings, the CATP combination therapy with mIL-12 and mutant mIL-18 resulted in better inhibition of tumor growth and 80% of mice being tumor free, compared to 60% with IL-12 alone, as shown in FIG. 9C and FIGS. 12D-12E. In summary, CATP combining IL-12 and mutant IL-18 demonstrates superior efficacy across multiple cancer models. Moreover, no overt body weight differences were detected between the combination and the vehicle control group (PBS) one week after dosing, although the combination treatment was associated with an initial decrease in body weight within 2-3 days after dosing, as shown in FIGS. 11C, 12C, and 12E.

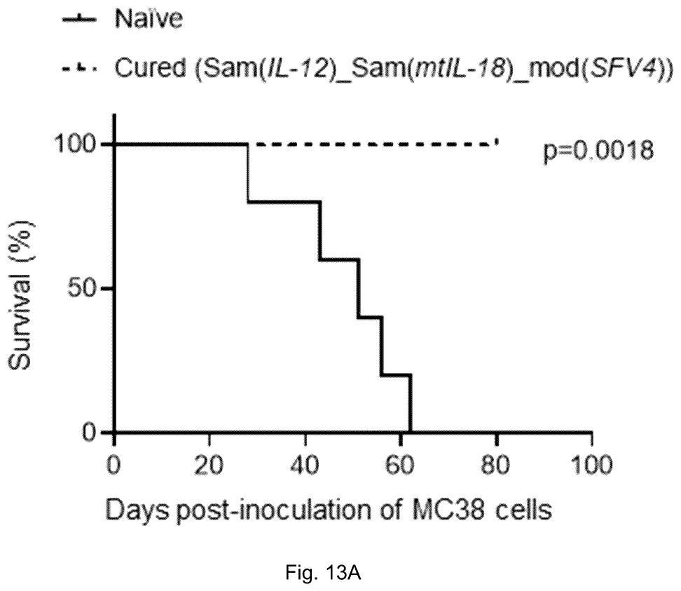

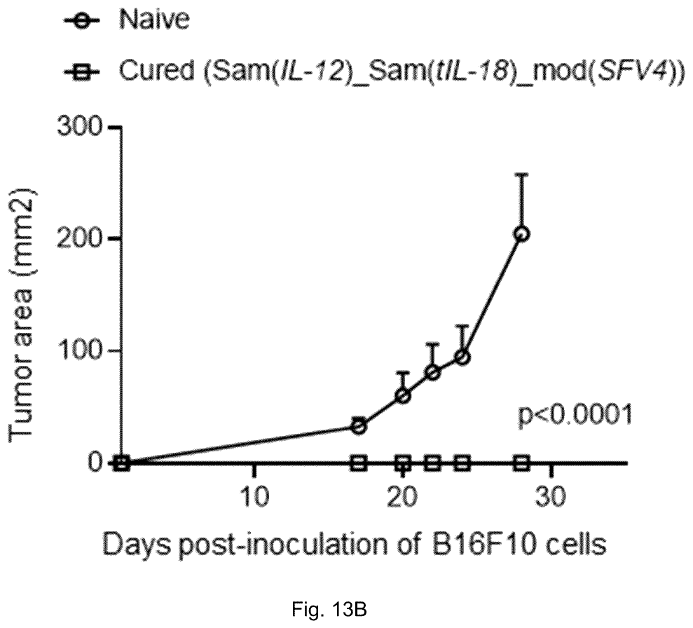

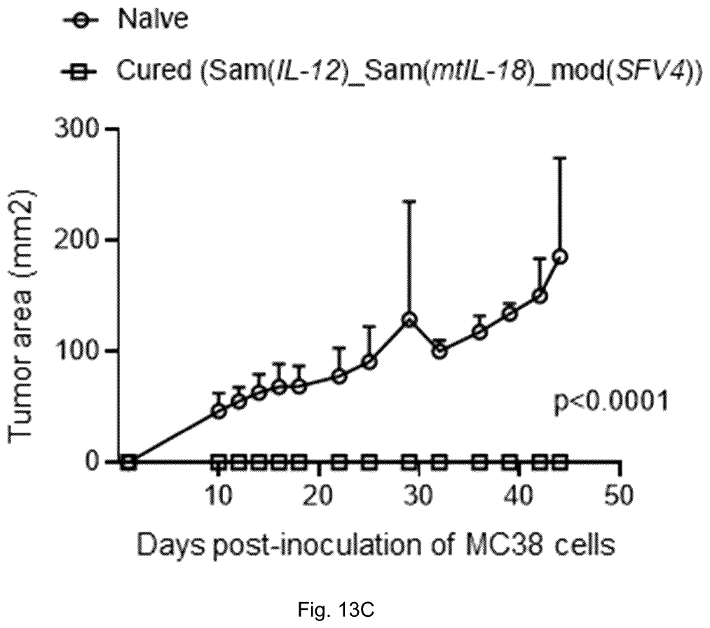

To determine whether tumor-free mice treated with the CATP combination therapy developed long-term immune memory in B16F10 and MC38 models, the cured mice were rechallenged with original tumor cells. Of note, all tumor-free mice were able to reject the tumor cells, in contrast to the age- and sex-matched naïve mice that developed tumors, as shown in FIG. 9D and FIGS. 13A-13C. These results suggested that the CATP system induces robust long-term immune memory. To investigate the mechanisms underlying this immune memory, B16F10 melanoma was intratumorally treated with the CATP therapy using mouse IL-12 alone, as shown in FIG. 4A. Flow cytometry analysis of tumor draining lymph nodes (TDLN) and spleen showed that CATP group significantly (p=0.0006) increased the number of memory precursor cells characterized by CD62L⁺ CD122⁺ CD8 T cells in TDLN, rather than in spleen, compared to the control treatment group, as shown in FIGS. 4A-4B. However, no significant differences were observed in CD62L⁻ KLRG1⁺ cytotoxic CD8 T¹⁹ cells either in TDLN or in spleen, as shown in FIG. 4C. These findings suggested that the CATP therapy likely promotes the increase of the memory precursor CD8 T cells, contributing to long term protection against tumor recurrence. A novel cascade amplification of therapeutic payloads (CATP) strategy was developed to overcome the challenges of achieving therapeutic payload thresholds in cancer therapy. The CATP system demonstrated a remarkable 525-fold increase in mouse IL-12 expression in the B16F10 melanoma model compared to conventional samRNA delivery methods. This innovative approach significantly enhances therapeutic potency, presenting a viable solution for gene therapies requiring high payloads without escalating dosage levels.

It was also shown that the CATP system co-expressing mouse IL-12 and a mutant form of IL-18 was effective against multiple cancer types, including B16F10 melanoma, MC38 and CT26 colon cancers, and P53^{null} K^{RasG12D} pancreatic duct cancer. Importantly, the system does not require personalized tumor-associated antigens, enabling its broad applicability across diverse malignancies. These findings position the CATP strategy as a promising platform for developing broad-spectrum cancer immunotherapies with robust therapeutic potential.

### Example 6: Production of sa-mRNA

A study was conducted to produce sa-mRNA constructs. In one aspect, the first nucleic construct is an sa-mRNA designed using the method described herein. Directed evolution of SAM001 and SAM002 (SEQ ID NOs: 58 and 59 respectively), encoding GFP (SEQ ID NO: 24) and puromycin resistance gene (puromycin) (SEQ ID NO: 68) according to Tables 1-2 below, was performed at different concentrations (1 ug/ml or 10 μg/ml) of puromycin in C2C12 cells for 60 days.

TABLE 1

| Puromycin (ug/ml) | Contigs | Nucleotide | Amino Acids | Region |
|---|---|---|---|---|
| 1 | 5 | G5795A | Glu to Lys | nsP4 |
| 10 | 2 | C2623A | Asp to Glu | nsP2 |
| 10 | 3 | T3309C | Met to Thr | nsP2 |
| 10 | 3 | A3493C | Glu to Asp | nsP2 |
| 10 | 3 | A3867G | Lys to Arg | nsP2 |
| 10 | 4 | A4674G | Lys to Arg | nsP3 |
| 10 | 5 | A5897G | Arg to Gly | nsP4 |

TABLE 2

| | | | | |
|---|---|---|---|---|
| Contigs | 2 | 3 | 4 | 5 |
| | WT | WT | WT | WT |
| | a1(C2623A) | b1(T3309C) | c1(4674G) | d1(A5897G) |
| | | b2(A3493C) | | |
| | | b3(A3867G) | | |
| Allele No | 2 | 4 | 2 | 2 |

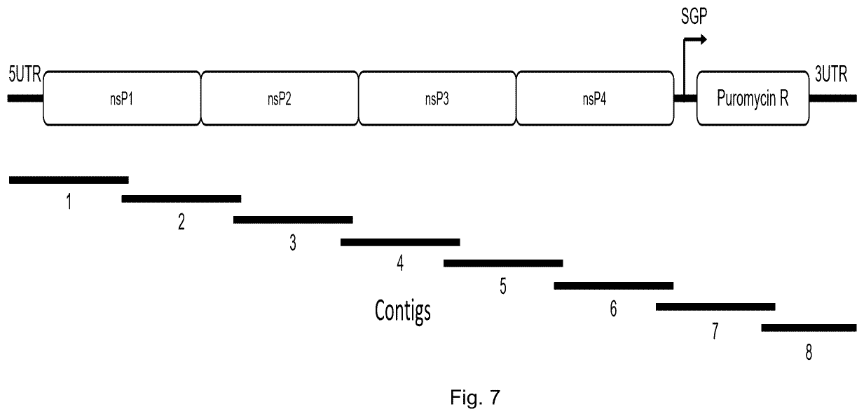

RNA dependent RNA polymerases are known to have a high error rate, which will cause mutants of SAM002 to appear over time. The SAM002 construct as shown in FIG. 7 was divided into 8 contigs, marked 1-8 on FIG. 7, comprising SEQ ID NOs. 60-67. The 8 overlapping contigs facilitate cloning to a vector form suitable for Sanger DNA sequencing. As shown in Table 1, only 1 mutation was found at nsP4 in 1 ug/ml puromycin. In contrast, there were 6 mutations found in nsP2, nsP3, and nsP4 at 10 ug/ml puromycin. The 6 mutations were in the 2^{nd}, 3^{rd}, 4^{th}, 5^{th} contigs. The 6 mutations of SAM002 is numbered as alleles 2, 4, 2, 2 respectively, which could form 32 variants. Tables 3-13 below show 66 variations of nucleic acid templates capable of producing 66 sa-mRNA variations produced using the method described above using SAM001 and SAM002 as a starting point, wherein the position of the mutations are counted from the first nucleotide of SEQ ID NOs: 58 (SAM001) or 59 (SAM002):

TABLE 3

| No. | | % of GFP normalized at day 3 | | |
|---|---|---|---|---|
| 1 | a1BCD-Sam002 | 93.60967 | 85.55008 | 92.43697479 |
| 2 | a1BCD-Sam001 | 26.3245 | 26.94064 | 24.56418384 |
| 3 | Ab1CD-Sam002 | 84.06015 | 83.50669 | 80.09049774 |
| 4 | Ab1CD-Sam001 | 20.36517 | 21.08844 | 21.40921409 |
| 5 | Ab2CD-Sam002 | 88.83792 | 89.1369 | 91.12781955 |
| 6 | Ab2CD-Sam001 | 35.46667 | 36.71875 | 32.23593964 |
| 7 | Ab3CD-Sam002 | 42.59259 | 41.76991 | 40.625 |
| 8 | Ab3CD-Sam001 | 7.362637 | 10.01764 | 8.549747049 |
| 9 | Abc1D-Sam002 | 75.67568 | 74.12224 | 72.98701299 |
| 10 | Abc1D-Sam001 | 25.06739 | 19.02174 | 21.32253711 |
| 11 | ABCd1-Sam002 | 88.4273 | 84.55657 | 85.80246914 |
| 12 | ABCd1-Sam001 | 34.73282 | 31.71355 | 36.14609572 |
| 13 | a1b1CD-Sam002 | 82.53968 | 75.84803 | 83.81962865 |
| 14 | a1b1CD-Sam001 | 24.43325 | 22.82472 | 18.86075949 |
| 15 | a1b2CD-Sam002 | 90.82707 | 85.01441 | 85.89928058 |
| 16 | a1b2CD-Sam001 | 43.10345 | 43 | 41.4004914 |
| 17 | a1b3CD-Sam002 | 47.98808 | 51.11441 | 51.30674003 |
| 18 | a1b3CD-Sam001 | 13.7415 | 11.36182 | 14.97975709 |
| 19 | a1Bc1D-Sam002 | 68.26265 | 70.17544 | 73.82550336 |
| 20 | a1Bc1D-Sam001 | 24.86842 | 26.52005 | 24.66843501 |
| 21 | a1BCd1-Sam002 | 81.64557 | 85.66929 | 87.59936407 |
| 22 | a1BCd1-Sam001 | 23.58621 | 28.09587 | 30.1759134 |

TABLE 4

| No. | | % of MFI normalized at day 3 | | |
|---|---|---|---|---|
| 1 | a1BCD-Sam002 | 26.70408 | 24.86716 | 28.17502279 |
| 2 | a1BCD-Sam001 | 92.66064 | 71.8318 | 69.01040305 |
| 3 | Ab1CD-Sam002 | 29.60338 | 27.95987 | 28.80892183 |
| 4 | Ab1CD-Sam001 | 54.84388 | 72.12059 | 59.69650342 |
| 5 | Ab2CD-Sam002 | 25.2417 | 22.28458 | 24.65648054 |
| 6 | Ab2CD-Sam001 | 79.93834 | 75.08098 | 69.93131997 |
| 7 | Ab3CD-Sam002 | 62.02842 | 59.2609 | 53.425 |
| 8 | Ab3CD-Sam001 | 72.97279 | 46.89674 | 60.26471613 |
| 9 | Abc1D-Sam002 | 44.37571 | 45.86502 | 40.95282024 |
| 10 | Abc1D-Sam001 | 80.15279 | 73.20619 | 82.89277065 |
| 11 | ABCd1-Sam002 | 19.66246 | 19.21909 | 18.82683057 |
| 12 | ABCd1-Sam001 | 64.61213 | 70.72639 | 68.27440117 |
| 13 | a1b1CD-Sam002 | 26.06985 | 28.32976 | 26.02497234 |
| 14 | a1b1CD-Sam001 | 63.195 | 56.67116 | 53.00767428 |
| 15 | a1b2CD-Sam002 | 21.32604 | 21.28533 | 21.99453112 |
| 16 | a1b2CD-Sam001 | 69.66121 | 71.22326 | 72.73373474 |
| 17 | a1b3CD-Sam002 | 60.85935 | 58.26164 | 56.95274307 |
| 18 | a1b3CD-Sam001 | 61.42157 | 45.29323 | 38.56780888 |
| 19 | a1Bc1D-Sam002 | 49.57803 | 46.91416 | 44.24096222 |
| 20 | a1Bc1D-Sam001 | 68.03935 | 78.72531 | 75.14674158 |
| 21 | a1BCd1-Sam002 | 18.37446 | 19.01418 | 19.03699816 |
| 22 | a1BCd1-Sam001 | 61.15648 | 70.99497 | 64.04494382 |

TABLE 5

| No. | | nsP3 transcripts normalized with Actin | | |
|---|---|---|---|---|
| 1 | a1BCD-Sam002 | 9.447941 | 12.38052 | 10.056107 |
| 2 | a1BCD-Sam001 | 51.62507 | 58.08123 | 49.8665331 |
| 3 | Ab1CD-Sam002 | 14.42001 | 24.59 | 25.4571675 |
| 4 | Ab1CD-Sam001 | 84.44851 | 119.4282 | 119.428223 |
| 5 | Ab2CD-Sam002 | 14.92853 | 16.91229 | 14.825409 |
| 6 | Ab2CD-Sam001 | 63.11889 | 64.89341 | 62.682899 |
| 7 | Ab3CD-Sam002 | 36.75835 | 41.35529 | 41.9325889 |
| 8 | Ab3CD-Sam001 | 177.294 | 199.4661 | 160.897712 |
| 9 | Abc1D-Sam002 | 19.42712 | 17.5087 | 16.336194 |
| 10 | Abc1D-Sam001 | 66.71781 | 57.68003 | 72.5045687 |
| 11 | ABCd1-Sam002 | 11.71269 | 10.48315 | 13.4543426 |
| 12 | ABCd1-Sam001 | 60.96883 | 58.89201 | 85.6273635 |
| 13 | a1b1CD-Sam002 | 17.75311 | 17.02992 | 24.7610399 |
| 14 | a1b1CD-Sam001 | 148.0561 | 121.9377 | 150.122874 |
| 15 | a1b2CD-Sam002 | 8.75435 | 8.815241 | 11.1579493 |
| 16 | a1b2CD-Sam001 | 7.568461 | 6.453134 | 9.44794129 |
| 17 | a1b3CD-Sam002 | 56.88593 | 47.83518 | 61.3929036 |
| 18 | a1b3CD-Sam001 | 182.2784 | 178.5272 | 163.14376 |
| 19 | a1Bc1D-Sam002 | 36.50444 | 37.27147 | 36.0018715 |
| 20 | a1Bc1D-Sam001 | 120.2589 | 124.4998 | 119.428223 |
| 21 | a1BCd1-Sam002 | 10.77787 | 11.47164 | 13.6421583 |
| 22 | a1BCd1-Sam001 | 27.09585 | 22.3159 | 27.09585 |

TABLE 6

| No. | | eGFP transcripts normalized with Actin | | |
|---|---|---|---|---|
| 1 | a1BCD-Sam002 | 5.133704 | 4.958831 | 4.40762046 |
| 2 | a1BCD-Sam001 | 37.79177 | 40.50421 | 30.4844159 |
| 3 | Ab1CD-Sam002 | 7.110741 | 10.05611 | 11.1579493 |
| 4 | Ab1CD-Sam001 | 62.6829 | 72.50457 | 66.7178087 |
| 5 | Ab2CD-Sam002 | 6.062866 | 5.61778 | 5.46416103 |
| 6 | Ab2CD-Sam001 | 41.64294 | 49.86653 | 44.323503 |
| 7 | Ab3CD-Sam002 | 21.55574 | 22.47112 | 24.2514651 |
| 8 | Ab3CD-Sam001 | 90.50967 | 92.41147 | 81.5718801 |
| 9 | Abc1D-Sam002 | 8.514961 | 6.868523 | 8.63382589 |
| 10 | Abc1D-Sam001 | 67.64915 | 56.49299 | 67.1818678 |
| 11 | ABCd1-Sam002 | 3.41054 | 3.24901 | 3.58010028 |
| 12 | ABCd1-Sam001 | 24.93327 | 26.72281 | 34.7755156 |
| 13 | a1b1CD-Sam002 | 6.543216 | 6.147501 | 7.3615012 |
| 14 | a1b1CD-Sam001 | 86.82268 | 76.10926 | 74.5429495 |
| 15 | a1b2CD-Sam002 | 3.116658 | 2.8481 | 3.07375036 |
| 16 | a1b2CD-Sam001 | 2.789487 | 2.584706 | 3.07375036 |
| 17 | a1b3CD-Sam002 | 25.81254 | 23.91759 | 25.281322 |
| 18 | a1b3CD-Sam001 | 51.62507 | 33.3589 | 41.3552906 |
| 19 | a1Bc1D-Sam002 | 12.81712 | 11.15795 | 10.5560633 |
| 20 | a1Bc1D-Sam001 | 50.21338 | 50.91434 | 47.176615 |
| 21 | a1BCd1-Sam002 | 2.694467 | 3.052518 | 3.97236998 |
| 22 | a1BCd1-Sam001 | 3.97237 | 3.917681 | 4.72397065 |

TABLE 7

| No. | | % of GFP normalized at day 3 | | |
|---|---|---|---|---|
| 23 | Ab1c1D-Sam002 | 57.94271 | 55.04711 | 58.37696335 |
| 24 | Ab1c1D-Sam001 | 14.20749 | 14.68531 | 12.45390071 |
| 25 | Ab2c1D-Sam002 | 70.09736 | 68.3844 | 68.66096866 |
| 26 | Ab2c1D-Sam001 | 20.82067 | 23.27965 | 21.19309262 |
| 27 | Ab3c1D-Sam002 | 34.7651 | 33.24397 | 35.53162853 |
| 28 | Ab3c1D-Sam001 | 13.96648 | 20.9589 | 13.76404494 |
| 29 | Ab1Cd1-Sam002 | 74.92308 | 76.68232 | 80.26101142 |
| 30 | Ab1Cd1-Sam001 | 15.24476 | 20.0569 | 13.22727273 |
| 31 | Ab2Cd1-Sam002 | 84.28571 | 82.7957 | 84.78964401 |
| 32 | Ab2Cd1-Sam001 | 33.70474 | 30.24251 | 27.28592163 |
| 33 | Ab3Cd1-Sam002 | 44.31138 | 39.58333 | 44.50704225 |
| 34 | Ab3Cd1-Sam001 | 19.94498 | 10.28037 | 10.68181818 |
| 35 | Abc1d1-Sam002 | 65.26868 | 67.29798 | 67.59847522 |
| 36 | Abc1d1-Sam001 | 22.30483 | 21.73397 | 23.50791717 |
| 37 | a1b1c1D-Sam002 | 51.07731 | 52.57069 | 51.30890052 |
| 38 | a1b1c1D-Sam001 | 18.37769 | 16.13723 | 15.2866242 |
| 39 | a1b2c1D-Sam002 | 63.92496 | 56.13772 | 57.55725191 |
| 40 | a1b2c1D-Sam001 | 18.06167 | 17.48148 | 16.79389313 |
| 41 | a1b3c1D-Sam002 | 18.5 | 19.86755 | 21.06109325 |
| 42 | a1b3c1D-Sam001 | 7.973761 | 10.42735 | 9.677891654 |
| 43 | a1b1Cd1-Sam002 | 73.34315 | 73.23308 | 75.65485362 |
| 44 | a1b1Cd1-Sam001 | 14.30536 | 13.49501 | 15.88652482 |

TABLE 8

| No. | | % of MFI normalized at day 3 | | |
|---|---|---|---|---|
| 23 | Ab1c1D-Sam002 | 50.962 | 48.41584 | 49.06840699 |
| 24 | Ab1c1D-Sam001 | 45.2422 | 52.21293 | 80.05868412 |
| 25 | Ab2c1D-Sam002 | 40.1987 | 36.34074 | 41.85967826 |
| 26 | Ab2c1D-Sam001 | 77.60194 | 64.59525 | 72.00743858 |
| 27 | Ab3c1D-Sam002 | 75.13296 | 69.43731 | 88.85059056 |
| 28 | Ab3c1D-Sam001 | 82.07372 | 41.10864 | 70.3764424 |
| 29 | Ab1Cd1-Sam002 | 21.75325 | 22.44224 | 21.7947142 |
| 30 | Ab1Cd1-Sam001 | 56.84459 | 57.05329 | 47.73401939 |
| 31 | Ab2Cd1-Sam002 | 16.99651 | 16.89642 | 15.9159919 |
| 32 | Ab2Cd1-Sam001 | 54.45878 | 52.79225 | 51.64851339 |
| 33 | Ab3Cd1-Sam002 | 47.26541 | 48.89045 | 41.55331882 |
| 34 | Ab3Cd1-Sam001 | 57.46982 | 80.52686 | 74.81101512 |
| 35 | Abc1d1-Sam002 | 38.89447 | 36.95033 | 39.74566762 |
| 36 | Abc1d1-Sam001 | 73.61894 | 61.88906 | 78.83554648 |
| 37 | a1b1c1D-Sam002 | 48.99976 | 52.12974 | 55.4643059 |
| 38 | a1b1c1D-Sam001 | 36.22922 | 41.67375 | 47.0700924 |
| 39 | a1b2c1D-Sam002 | 40.58023 | 43.91632 | 45.09061489 |
| 40 | a1b2c1D-Sam001 | 77.31859 | 68.19837 | 66.24362946 |
| 41 | a1b3c1D-Sam002 | 83.91999 | 67.79338 | 66.58917612 |
| 42 | a1b3c1D-Sam001 | 47.01905 | 34.42404 | 37.22906315 |
| 43 | a1b1Cd1-Sam002 | 25.29739 | 23.40211 | 23.24178645 |
| 44 | a1b1Cd1-Sam001 | 83.93086 | 70.15867 | 53.45713157 |

TABLE 9

| No. | | nsP3 transcripts normalized with Actin | | |
|---|---|---|---|---|
| 23 | Ab1c1D-Sam002 | 50.21338 | 43.71329 | 46.5271206 |
| 24 | Ab1c1D-Sam001 | 79.89316 | 81.57188 | 79.3412928 |
| 25 | Ab2c1D-Sam002 | 36.50444 | 35.0174 | 37.5307184 |
| 26 | Ab2c1D-Sam001 | 115.3601 | 106.8913 | 107.634741 |
| 27 | Ab3c1D-Sam002 | 37.27147 | 41.06963 | 41.6429394 |
| 28 | Ab3c1D-Sam001 | 69.55103 | 77.1717 | 62.2499166 |
| 29 | Ab1Cd1-Sam002 | 27.85762 | 29.65082 | 27.09585 |
| 30 | Ab1Cd1-Sam001 | 163.1438 | 172.4459 | 178.527189 |
| 31 | Ab2Cd1-Sam002 | 34.29675 | 32.89964 | 33.1284776 |
| 32 | Ab2Cd1-Sam001 | 166.5718 | 155.4169 | 148.056088 |
| 33 | Ab3Cd1-Sam002 | 0.752623 | 0.779165 | 0.83508792 |
| 34 | Ab3Cd1-Sam001 | 1.536875 | 1.231144 | 1.26575659 |
| 35 | Abc1d1-Sam002 | 2.770219 | 2.496661 | 2.37841423 |
| 36 | Abc1d1-Sam001 | 147.0334 | 132.5139 | 142.024892 |
| 37 | a1b1c1D-Sam002 | 91.13921 | 96.33579 | 95.0095085 |
| 38 | a1b1c1D-Sam001 | 191.3407 | 207.9366 | 196.720023 |
| 39 | a1b2c1D-Sam002 | 15.45498 | 14.6213 | 19.6983106 |
| 40 | a1b2c1D-Sam001 | 101.1253 | 101.8287 | 113.771863 |
| 41 | a1b3c1D-Sam002 | 0.447513 | 0.397768 | 0.42337266 |
| 42 | a1b3c1D-Sam001 | 116.1625 | 124.4998 | 133.435617 |
| 43 | a1b1Cd1-Sam002 | 22.7848 | 22.62742 | 24.5900029 |
| 44 | a1b1Cd1-Sam001 | 210.8393 | 210.8393 | 207.936613 |

TABLE 10

| No. | | eGFP transcripts normalized with Actin | | |
|---|---|---|---|---|
| 23 | Ab1c1D-Sam002 | 16.44982 | 16 | 16.2233517 |
| 24 | Ab1c1D-Sam001 | 38.05463 | 41.64294 | 44.323503 |
| 25 | Ab2c1D-Sam002 | 11.47164 | 12.90627 | 14.5203065 |
| 26 | Ab2c1D-Sam001 | 49.86653 | 48.50293 | 51.9841534 |
| 27 | Ab3c1D-Sam002 | 9.063071 | 6.19026 | 6.58872814 |
| 28 | Ab3c1D-Sam001 | 21.55574 | 20.39297 | 19.0273138 |
| 29 | Ab1Cd1-Sam002 | 3.41054 | 3.5801 | 2.65737163 |
| 30 | Ab1Cd1-Sam001 | 26.72281 | 32.89964 | 37.7917652 |
| 31 | Ab2Cd1-Sam002 | 3.340352 | 3.530812 | 3.36358566 |
| 32 | Ab2Cd1-Sam001 | 21.40684 | 17.75311 | 18.5070109 |
| 33 | Ab3Cd1-Sam002 | 0.005486 | 0.005839 | 0.03564887 |
| 34 | Ab3Cd1-Sam001 | 0.00982 | 0.008729 | 0.00872881 |
| 35 | Abc1d1-Sam002 | 0.011438 | 0.010167 | 0.01045256 |
| 36 | Abc1d1-Sam001 | 10.41073 | 7.727491 | 10.6294865 |
| 37 | a1b1c1D-Sam002 | 24.42015 | 20.39297 | 35.2609637 |
| 38 | a1b1c1D-Sam001 | 86.22295 | 89.88447 | 72.5045687 |
| 39 | a1b2c1D-Sam002 | 0.406126 | 0.539614 | 0.70710678 |
| 40 | a1b2c1D-Sam001 | 68.11969 | 76.10926 | 69.0706071 |
| 41 | a1b3c1D-Sam002 | 0.004944 | 0.004072 | 0.00315094 |
| 42 | a1b3c1D-Sam001 | 134.3637 | 140.0696 | 138.141214 |
| 43 | a1b1Cd1-Sam002 | 4.890561 | 4.563055 | 5.16941132 |
| 44 | a1b1Cd1-Sam001 | 50.21338 | 49.18001 | 46.5271206 |

TABLE 11

| No. | | % of GFP normalized at day 3 | | |
|---|---|---|---|---|
| 45 | a1b2Cd1-Sam002 | 86.72087 | 85.7337 | 85.2367688 |
| 46 | a1b2Cd1-Sam001 | 29.46429 | 27.00922 | 31.53846154 |
| 47 | a1b3Cd1-Sam002 | 37.07025 | 36.80124 | 37.57668712 |
| 48 | a1b3Cd1-Sam001 | 5.844828 | 6.109091 | 6.763110307 |
| 49 | Ab1c1d1-Sam002 | 42.32633 | 37.85124 | 41.06870229 |
| 50 | Ab1c1d1-Sam001 | 9.556314 | 5.738832 | 7.123966942 |
| 51 | Ab2c1d1-Sam002 | 60.93294 | 60 | 60.34732272 |
| 52 | Ab2c1d1-Sam001 | 22.2964 | 16.52893 | 20.51282051 |
| 53 | Ab3c1d1-Sam002 | 30.11583 | 30.46272 | 22.75132275 |
| 54 | Ab3c1d1-Sam001 | 9.871959 | 9.257362 | 9.638242894 |
| 55 | a1Bc1d1-Sam002 | 53.46535 | 49.91334 | 58.52842809 |
| 56 | a1Bc1d1-Sam001 | 11.69697 | 11.47692 | 13.87442573 |
| 57 | a1b1c1d1-Sam002 | 45.08929 | 40.68554 | 38.14102564 |
| 58 | a1b1c1d1-Sam001 | 12.20979 | 12.54795 | 21.51724138 |
| 59 | a1b2c1d1-Sam002 | 68.81579 | 70.96774 | 67.578125 |
| 60 | a1b2c1d1-Sam001 | 21.31783 | 21.50259 | 25.89641434 |
| 61 | a1b3c1d1-Sam002 | 26.46675 | 24.3807 | 29.17214192 |
| 62 | a1b3c1d1-Sam001 | 9.371795 | 10.38119 | 17.31984829 |
| 63 | ABCD-Sam001 | 42.87516 | 41.27182 | 47.11779449 |
| 64 | ABCD-Sam002 | 88.08777 | 85.36585 | 87.59231905 |
| 65 | ABCd2-Sam002 | 76.58897 | 82.50433 | 90.74117236 |
| 66 | ABCd2-Sam001 | 25.07269 | 23.83486 | 27.81926965 |

TABLE 12

| No. | | % of MFI normalized at day 3 | | |
|---|---|---|---|---|
| 45 | a1b2Cd1-Sam002 | 21.59581 | 21.38896 | 21.2904946 |
| 46 | a1b2Cd1-Sam001 | 80.67199 | 62.76361 | 60.79245886 |
| 47 | a1b3Cd1-Sam002 | 59.51047 | 50.75597 | 49.29017604 |
| 48 | a1b3Cd1-Sam001 | 37.66094 | 100.0263 | 53.2477737 |
| 49 | Ab1c1d1-Sam002 | 46.83513 | 47.13787 | 50.37907506 |
| 50 | Ab1c1d1-Sam001 | 59.35291 | 51.54369 | 51.29244805 |
| 51 | Ab2c1d1-Sam002 | 49.5077 | 41.19688 | 43.92891221 |
| 52 | Ab2c1d1-Sam001 | 54.3385 | 71.99053 | 72.05443699 |
| 53 | Ab3c1d1-Sam002 | 68.90104 | 77.1418 | 86.83062969 |
| 54 | Ab3c1d1-Sam001 | 97.9473 | 55.35554 | 49.14956012 |
| 55 | a1Bc1d1-Sam002 | 47.22042 | 38.97181 | 40.27795818 |
| 56 | a1Bc1d1-Sam001 | 55.91852 | 77.0978 | 55.41929666 |
| 57 | a1b1c1d1-Sam002 | 45.95797 | 40.88739 | 55.26597644 |
| 58 | a1b1c1d1-Sam001 | 39.12734 | 47.70419 | 30.85771948 |
| 59 | a1b2c1d1-Sam002 | 36.97674 | 36.20338 | 35.07727652 |
| 60 | a1b2c1d1-Sam001 | 61.17269 | 58.38926 | 53.09734513 |
| 61 | a1b3c1d1-Sam002 | 77.3002 | 69.05956 | 44.25943546 |
| 62 | a1b3c1d1-Sam001 | 54.34123 | 134.8006 | 109.557945 |
| 63 | ABCD-Sam001 | 75.08005 | 63.53236 | 58.12901796 |
| 64 | ABCD-Sam002 | 25.37244 | 25.65748 | 24.13470845 |
| 65 | ABCd2-Sam002 | 22.24231 | 25.40835 | 20.73913043 |
| 66 | ABCd2-Sam001 | 87.19298 | 79.77941 | 87.31617647 |

TABLE 13

| No. | | nsP3 transcripts normalized with Actin | | |
|---|---|---|---|---|
| 45 | a1b2Cd1-Sam002 | 41.06963 | 36.75835 | 35.2609637 |
| 46 | a1b2Cd1-Sam001 | 128 | 137.187 | 121.937664 |
| 47 | a1b3Cd1-Sam002 | 49.18001 | 51.98415 | 48.8402947 |
| 48 | a1b3Cd1-Sam001 | 136.2394 | 146.0178 | 165.421162 |
| 49 | Ab1c1d1-Sam002 | 60.54769 | 63.55792 | 70.5219274 |
| 50 | Ab1c1d1-Sam001 | 257.7806 | 243.8753 | 266.871235 |
| 51 | Ab2c1d1-Sam002 | 38.58585 | 43.71329 | 42.2242531 |
| 52 | Ab2c1d1-Sam001 | 62.24992 | 69.55103 | 71.5063768 |
| 53 | Ab3c1d1-Sam002 | 56.49299 | 60.96883 | 60.5476894 |
| 54 | Ab3c1d1-Sam001 | 139.1021 | 141.0439 | 141.043855 |
| 55 | a1Bc1d1-Sam002 | 26.90869 | 31.55945 | 31.3414495 |
| 56 | a1Bc1d1-Sam001 | 58.48521 | 64 | 62.682899 |
| 57 | a1b1c1d1-Sam002 | 29.04061 | 34.29675 | 39.3966212 |
| 58 | a1b1c1d1-Sam001 | 103.2501 | 109.1373 | 135.298309 |
| 59 | a1b2c1d1-Sam002 | 27.47409 | 26.53823 | 28.0513831 |
| 60 | a1b2c1d1-Sam001 | 60.12946 | 74.02804 | 66.2569551 |
| 61 | a1b3c1d1-Sam002 | 51.26847 | 55.71524 | 58.4852128 |
| 62 | a1b3c1d1-Sam001 | 128.8903 | 129.7868 | 132.51391 |
| 63 | ABCD-Sam001 | 69.07061 | 61.3929 | 63.1188931 |
| 64 | ABCD-Sam002 | 13.92881 | 13.8326 | 9.64646262 |
| 65 | ABCd2-Sam002 | 47.17662 | 47.50475 | 44.0173382 |
| 66 | ABCd2-Sam001 | 13.08643 | 13.64216 | 15.1369223 |

TABLE 14

| No. | | eGFP transcripts normalized with Actin | | |
|---|---|---|---|---|
| 45 | a1b2Cd1-Sam002 | 1.931873 | 1.292353 | 1.86606598 |
| 46 | a1b2Cd1-Sam001 | 30.90996 | 28.44297 | 23.7523771 |
| 47 | a1b3Cd1-Sam002 | 16.56424 | 17.14838 | 13.3614067 |
| 48 | a1b3Cd1-Sam001 | 48.50293 | 56.49299 | 61.8199251 |
| 49 | Ab1c1d1-Sam002 | 14.3204 | 15.24221 | 15.5624792 |
| 50 | Ab1c1d1-Sam001 | 80.44886 | 81.57188 | 75.5835303 |
| 51 | Ab2c1d1-Sam002 | 9.189587 | 10.05611 | 8.9382971 |
| 52 | Ab2c1d1-Sam001 | 32 | 35.50622 | 35.0173984 |
| 53 | Ab3c1d1-Sam002 | 28.05138 | 26.17287 | 28.6408023 |
| 54 | Ab3c1d1-Sam001 | 49.18001 | 36.50444 | 48.1678959 |
| 55 | a1Bc1d1-Sam002 | 14.22148 | 12.04197 | 11.7941537 |
| 56 | a1Bc1d1-Sam001 | 37.01402 | 36.25228 | 30.6964518 |
| 57 | a1b1c1d1-Sam002 | 13.36141 | 14.52031 | 18.6357374 |
| 58 | a1b1c1d1-Sam001 | 61.3929 | 64 | 75.0614368 |
| 59 | a1b2c1d1-Sam002 | 9.646463 | 8.815241 | 8.6938789 |
| 60 | a1b2c1d1-Sam001 | 32.89964 | 34.77552 | 33.8245773 |
| 61 | a1b3c1d1-Sam002 | 22.16175 | 23.75238 | 25.281322 |
| 62 | a1b3c1d1-Sam001 | 71.01245 | 64.89341 | 71.0124462 |
| 63 | ABCD-Sam001 | 35.50622 | 22.94328 | 27.857618 |
| 64 | ABCD-Sam002 | 14.22148 | 5.897077 | 6.45313407 |
| 65 | ABCd2-Sam002 | 44.6318 | 40.78594 | 30.4844159 |
| 66 | ABCd2-Sam001 | 6.868523 | 7.310652 | 8.45614432 |

Tables 3-14 show characterizations of 66 sa-mRNA mutants. To generate these mutants, C2C12 cells were transfected with SAM002 encoding puromycin resistance gene and GFP encapsulated by an LNP comprising the ionizable lipid P6 (P6-LNP). The transfected cells were cultured for 2 months at 1 or 10 μg/ml puromycin. At 2 months post transfection, the total RNA of selected cells was extracted and reverse transcribed. The specific primers covering contigs from 1 to 6 were for amplicons and sub-cloning. For each contig, 8 clones were cultured and isolated using a Mini-Prep procedure to isolate small plasmid DNA from bacteria while limiting contaminating proteins and genomic DNA for Sanger Sequencing. The contig sequences comprise SEQ ID NOs. 60-67, which correspond to contigs 1-8, respectively. The identified mutations could make 66 combinations and were further engineered into SAM001 or SAM002 at the specified location for each mutation. Thus, this study identified 67 constructs of cytopathic and noncytopathic sa-mRNA, including the mutation at 1 μg/ml puromycin and the 66 mutants shown in Tables 3-14.

A characterization study was conducted to study the expression level of the 66 sa-mRNA variants identified using the method described above. The 66 sa-mRNA variants were transcribed in vitro and transfected to C2C12 cells using a LNP comprising an ionizable lipid P6 as defined in PCT Patent Application No. PCT/US2023/017777 (P6-LNP), which is fully incorporated herein by reference. The transfected cells were performed by fluorescence-activated cell sorting (FACS) at day 1 and 3 post transfection. The percentages of GFP and mean fluorescent intensities (MFI), representing gene product expression of each variant, were analyzed. The percentage and MFI of GFP at day 3 were normalized compared to the data from day 1. Total RNA of the transfected cells was extracted and reverse transcribed as complementary DNA for quantification polymerase chain reaction (qPCR) by specific probes nsP3 and eGFP.

To characterize the identified variants overtime, the variants were transfected using P6-LNP into mouse myoblast C2C12 cells, analyzed by flow cytometer at day 1 and 3 post transfection, and quantified the transcript number of each sa-mRNA construct using nsP3 specific probes. The subgenomic transcripts were quantified using GFP specific probes. The decrease of GFP cells and intensity of GFP ranged broadly between the tested variants.

In one aspect, the first nucleic construct is an sa-mRNA comprising one or more mutations compared to a sa-mRNA construct encoding a GOI (SEQ ID NO: 41, 42, 73, 74, 75, or 76) according to Table 15 below, wherein the position of the mutations are counted from the first nucleotide of each sequence:

TABLE 15

| | Nucleotide Change Compared to SEQ ID NO: 41, 42, |
|---|---|
| | 73, 74, 75, or 76 |
| Mutation 1 | C2623A |
| Mutation 2 | T3309C |
| Mutation 3 | A3493C |
| Mutation 4 | A3867G |
| Mutation 5 | A4674G |
| Mutation 6 | A5897G |

TABLE 16
SEQUENCES:

| | SEQ | |
|---|---|---|
| | ID | |
| Description | NO. | Sequence |
| SGP | 1 | taacctgaatggactacgacatagtctagtccgccaag |
| | 2 | gaacttccatcatagttatggccatgactactctagctagcagtgttaaat |
| | | cattcagctacctgagaggggcccctataactctctacggc |
| | 3 | atggactacgacatagtctagtccgccaag |
| Murine | 4 | atgtgggagcttgaaaaagacgtctatgtagtagaagtggactggacacct |
| interleukin-12 | | gatgctcctggcgagacagttaacctcacatgcgatacccctgaggaagat |
| comprising | | gatatcacctggacttctgaccagagacacggggtgattgggagcggcaaa |
| heavy chain | | accctgacgatcactgtgaaggagtttctggacgccggccagtatacctgt |
| p40 (mIL12 | | cacaaggggggggagaccctgagtcatagccacctgttgctgcacaagaag |
| P40) | | gagaatggcatctggtctacagagatcctgaagaactttaagaacaagacc |
| | | ttcctgaagtgtgaagcaccaaactacagtggtcgctttacctgcagctgg |
| | | ctggtccaaagaaacatggacctgaaatttaatataaagagtagctcttcg |
| | | agtcctgattccagggccgtgacgtgcggcatggcaagcctttcagccgaa |
| | | aaagtcacgctggatcagcgagactatgagaagtacagcgttagctgtcag |
| | | gaggacgtaacttgcccgactgccgaggagactctgcccatagagctcgct |
| | | ctggaggccaggcagcagaacaaatatgagaattacagcactagtttcttt |
| | | attagagacatcatcaaacccgacccacccaagaatctgcagatgaagccg |
| | | ctgaagaatagtcaggtcgaggtttcctgggaatatccagattcatggtcc |
| | | actccgcattcttatttttccttaaaattctttgttaggattcagcggaaa |
| | | aaagaaaagatgaaagagacggaggaagggtgcaaccagaagggggccttc |
| | | ctggtggaaaagacaagcactgaggtccaatgtaagggtgggaacgtttgc |
| | | gtgcaggctcaggatcgctactacaacagcagttgctctaagtgggcctgc |
| | | gtaccttgtcgcgtcaggagt |
| Murine | 5 | agggtgatcccagtgtctgggccggcccgttgcttgtctcaatccagaaac |
| interleukin-12 | | ctcctcaagaccactgacgatatggtaaagactgcccgagagaagctaaaa |
| comprising | | cactactcttgtacagctgaagatatagaccatgaggatataacacgggac |
| light chain p35 | | cagacctctactctgaaaacctgtctgcctcttgagctgcacaagaacgag |
| (mIL12-P35) | | tcctgtctggctacccgcgaaacctcaagcacaaccagaggtagttgcctg |
| | | cccccacaaaagacatcgcttatgatgaccttgtgtctgggatctatttat |
| | | gaggacctgaagatgtaccaaactgagttccaggcaataaatgctgctctc |
| | | cagaatcacaatcatcaacaaatcatccttgataaggggatgctggtcgca |
| | | atcgacgagctcatgcaatcgctgaaccacaatggggaaaccctcaggcag |
| | | aaaccaccggtgggagaggccgacccctaccgtgttaaaatgaagttgtgt |
| | | attcttttgcatgcattctctacaagagtcgttaccatcaatcgcgtcatg |
| | | gggtacctgtcatcagcc |
| Murine | 6 | gggtacctgtcatcagccggcggtagtggtggtgggagccacaagtcctcc |
| interleukin-21 | | ccccagggtccggatcggctcttgatcagactgagacatctgattgatatt |
| (mIL21) | | gtcgagcagttgaagatctatgagaatgacctcgatcctgagttactgagt |
| | | gccccacaggacgttaaagggcactgtgaacacgccgcatttgcttgtttt |
| | | cagaaggccaagctgaaaccttctaatcccgggaataacaaaactttcatt |
| | | atcgatctcgtcgcgcagctgaggcggcgacttcctgcacggcgggggggg |
| | | aaaaagcaaaagcacatcgcaaagtgtccctcatgcgactcttacgagaaa |
| | | cgtacccctaaggagttccttgaaagactcaaatggctgctgcaaaagatg |
| | | atccaccagcatctcagc |
| Human | 7 | atatgggagctgaagaaggacgtgtatgtcgtggagctggactggtaccca |
| interleukin-12 | | gatgctcctggcgaaatggtggttttaacatgtgatacccccgaggaggac |
| comprising | | ggcatcacatggactctggaccagagttctgaggtgctggggtccggcaag |
| heavy chain | | actctgacaatccaggttaaggagttcggcgacgcaggacagtacacttgt |
| p40 (hIL12 | | cacaagggaggtgaggtgctttctcacagcctgttgctgctccataagaag |
| P40) | | gaagacggtatttggtcaaccgacatcctcaaggaccagaaggagcccaaa |
| | | aacaagacctttctgagatgtgaggccaagaattacagcggtagattcact |
| | | tgttggtggctcaccaccatatccacagacttgaccttcagtgtcaaaagt |
| | | tcacgagggagctcagatcctcaaggcgttacctgtggcgcagcgacgctg |
| | | tccgcagaaagagtcaggggagacaacaaggaatacgagtactctgtcgag |
| | | tgccaggaggattccgcctgtccggccgcagaggagtctttacctattgag |
| | | gtgatggtcgatgccgtgcacaagcttaagtacgagaattacacatcaagt |
| | | tttttcatccgcgacatcattaaacctgatccaccaaagaacctgcagctc |
| | | aagcctctgaagaatagcaggcaggtcgaggtaagctgggagtatcctgat |
| | | acctggtccaccccccacagttatttcagcctcaccttctgcgtccaagtc |
| | | cagggaaagagcaagagagagaagaaggatagggtgttcacagataagact |
| | | tcagctactgtgatctgcagaaagaatgcgtctatctctgtgcgagcacaa |
| | | gacaggtactacagttctagctggagcgagtgggcatcagtcccctgcagt |
| Human | 8 | aggaacctcccagttgctacacctgacccgggaatgtttccatgcctccac |
| interleukin-12 | | cattcccagaatctcctccgagccgtgtccaatatgctgcaaaaggctcgg |
| comprising | | cagaccttggagttttacccttgcacctcagaagaaatcgatcatgaggat |
| light chain p35 | | atcacaaaggataagacgagcactgttgaggcatgcctgcccctggagcta |
| (hIL12-P35) | | actaagaatgagtcttgcctgaacagcagggagacttccttcattaccaac |
| | | ggtagctgtcttgccagcaggaagacatcttttatgatggccctgtgtcta |
| | | tctagcatatatgaagacctgaagatgtaccaggtggaattcaaaaccatg |
| | | aatgctaagcttctcatggatcccaagaggcaaatcttcctggaccagaat |
| | | atgcttgctgtcatagatgaactgatgcaggcgttgaattttaacagcgag |
| | | acggtgcctcaaaaaagctcactggaagaacctgatttttataaaacgaag |
| | | atcaagctgtgtattttactacacgcctttagaatccgcgctgttaccatc |
| | | gacagagtaatgtcctacctaaatgcttca |
| Human | 9 | caggacaggcatatgatccggatgcggcagctgatcgatattgtagaccag |
| interleukin-21 | | ttgaagaattatgtgaacgacttagtgccggaattcctccccgcccccgag |
| (hIL21) | | gacgtggagactaattgtgagtggtctgcattctcatgcttccaaaaagca |
| | | cagctgaagagtgccaataccggcaataacgaaaggatcatcaatgtaagt |
| | | ataaagaagttaaaacgcaaaccgcccagtaccaacgctggacgcaggcaa |
| | | aaacacaggctgacatgcccctcgtgtgattcgtacgaaaaaaaacctcca |
| | | aaggaattcctggaaaggttcaagtccttattacagaaaatgattcaccag |
| | | cacctgagtagtaggacccacggatccgaagactcc |
| ORI | 10 | tttccataggctccgcccccctgacgagcatcacaaaaatcgacgctcaag |
| | | tcagaggtggcgaaacccgacaggactataaagataccaggcgtttccccc |
| | | tggaagctccctcgtgcgctctcctgttccgaccctgccgcttaccggata |
| | | cctgtccgcctttctcccttcgggaagcgtggcgctttctcatagctcacg |
| | | ctgtaggtatctcagttcggtgtaggtcgttcgctccaagctgggctgtgt |
| | | gcacgaaccccccgttcagcccgaccgctgcgccttatccggtaactatcg |
| | | tcttgagtccaacccggtaagacacgacttatcgccactggcagcagccac |
| | | tggtaacaggattagcagagcgaggtatgtaggcggtgctacagagttctt |
| | | gaagtggtggcctaactacggctacactagaagaacagtatttggtatctg |
| | | cgctctgctgaagccagttaccttcggaaaaagagttggtagctcttgatc |
| | | cggcaaacaaaccaccgctggtagcggtggtttttttgtttgcaagcagca |
| | | gattacgcgcagaaaaaaaggatctcaa |
| First | 11 | atgagtaacctgaggctatggcagggcctgccgccccgacgttggctgcga |
| Expression | | gccctgggccttcacccgaacttggggggtggggtggggaaaaggaagaaa |
| Unit | | cgcgggcgtattggccccaatggggtctcggtggggtatcgacagagtgcc |
| (Kana/Neo | | agccctgggaccgaaccccgcgtttatgaacaaacgacccaacaccgtgcg |
| Cassette) | | ttttattctgtctttttattgccgtcatagcgcgggttccttccggtattg |
| | | tctccttccgtgtttcagttagcctccccctagggtgggcgaagaactcca |
| | | gcatgagatccccgcgctggaggatcatccagccggcgtcccggaaaacga |
| | | ttccgaagcccaacctttcatagaaggcggcggtggaatcgaaatctcgtg |
| | | atggcaggttgggcgtcgcttggtcggtcatttcgaaccccagagtcccgc |
| | | tcagaagaactcgtcaagaaggcgatagaaggcgatgcgctgcgaatcggg |
| | | agcggcgataccgtaaagcacgaggaagcggtcagcccattcgccgccaag |
| | | ctcttcagcaatatcacgggtagccaacgctatgtcctgatagcgatccgc |
| | | cacacccagccggccacagtcgatgaatccagaaaagcggccattttccac |
| | | catgatattcggcaagcaggcatcgccatgggtcacgacgagatcctcgcc |
| | | gtcgggcatgctcgccttgagcctggcgaacagttcggctggcgcgagccc |
| | | ctgatgctcttcgtccagatcatcctgatcgacaagaccggcttccatccg |
| | | agtacgtgctcgctcgatgcgatgtttcgcttggtggtcgaatgggcaggt |
| | | agccggatcaagcgtatgcagccgccgcattgcatcagccatgatggatac |
| | | tttctcggcaggagcaaggtgagatgacaggagatcctgccccggcacttc |
| | | gcccaatagcagccagtcccttcccgcttcagtgacaacgtcgagcacagc |
| | | tgcgcaaggaacgcccgtcgtggccagccacgatagccgcgctgcctcgtc |
| | | ttgcagttcattcagggcaccggacaggtcggtcttgacaaaaagaaccgg |
| | | gcgcccctgcgctgacagccggaacacggcggcatcagagcagccgattgt |
| | | ctgttgtgcccagtcatagccgaatagcctctccacccaagcggccggaga |
| | | acctgcgtgcaatccatcttgttcaatcatgcgaaacgatcctcatcctgt |
| | | ctcttgatcgatctttgcaaaagcctaggcctccaaaaaagcctcctcact |
| | | acttctggaatagctcagaggccgaggcggcctcggcctctgcataaataa |
| | | aaaaaattagtcagccatggggcggagaatgggcggaactgggcggagtta |
| | | ggggcgggatgggcggagttaggggcgggactatggttgctgactaattga |
| | | gatgcatgctttgcatacttctgcctgctggggagcctggggactttccac |
| | | acctggttgctgactaattgagatgcatgctttgcatacttctgcctgctg |
| | | gggagcctggggactttccacaccctaactgacacacattccacagctggt |
| | | tctttccgcctcaggactcttcctttttcaatattattgaagcatttatca |
| | | gggttattgtctcatgagcggatacatatttgaatgtatttagaaaaataa |
| | | acaaataggggttccgcg |
| First | 12 | atgagtaaacttggtctgacagttaccaatgcttaatcagtgaggcaccta |
| Expression | | tctcagcgatctgtctatttcgttcatccatagttgcctgactccccgtcg |
| Unit (Amp | | tgtagataactacgatacgggagggcttaccatctggccccagtgctgcaa |
| Cassette) | | tgataccgcgagacccacgctcaccggctccagatttatcagcaataaacc |
| | | agccagccggaagggccgagcgcagaagtggtcctgcaactttatccgcct |
| | | ccatccagtctattaattgttgccgggaagctagagtaagtagttcgccag |
| | | ttaatagtttgcgcaacgttgttgccattgctacaggcatcgtggtgtcac |
| | | gctcgtcgtttggtatggcttcattcagctccggttcccaacgatcaaggc |
| | | gagttacatgatcccccatgttgtgcaaaaaagcggttagctccttcggtc |
| | | ctccgatcgttgtcagaagtaagttggccgcagtgttatcactcatggtta |
| | | tggcagcactgcataattctcttactgtcatgccatccgtaagatgctttt |
| | | ctgtgactggtgagtactcaaccaagtcattctgagaatagtgtatgcggc |
| | | gaccgagttgctcttgcccggcgtcaatacgggataataccgcgccacata |
| | | gcagaactttaaaagtgctcatcattggaaaacgttcttcggggcgaaaac |
| | | tctcaaggatcttaccgctgttgagatccagttcgatgtaacccactcgtg |
| | | cacccaactgatcttcagcatcttttactttcaccagcgtttctgggtgag |
| | | caaaaacaggaaggcaaaatgccgcaaaaaagggaataagggcgacacgga |
| | | aatgttgaatactcatactcttcctttttcaatattattgaagcatttatc |
| | | agggttattgtctcatgagcggatacatatttgaatgtatttagaaaaata |
| | | aacaaataggggttccgcg |
| Modified T7 | 13 | taatacgactcactataggatagg |
| Promoter | | |
| Wildtype T7 | 14 | taatacgactcactatagg |
| Promoter | | |
| SP6 promoter | 82 | atttaggtgacac |
| Kil RNAP | 83 | aattagggcacac |
| promoter | | |
| 5′ UTR | 15 | cggcgcatgagagaagcccagaccaattacctacccaaa |
| 5′ UTR | 16 | ttcgaaggcgcgcctctagagccacc |
| 5′ UTR | 17 | gcggcgcatgagagaagcccagaccaattacctacccaaa |
| 5′ UTR | 18 | acatttgcttctgacacaactgtgttcactagcaacctcaaacagacacc |
| 5′ UTR (human | 71 | accgccgagaccgcgtccgccccgcgagcacagagcctcgcctttgccgat |
| actin) | | ccgccgcccgtccacacccgccgccagctcaccatggat |
| 5′ UTR | 72 | ctagcttttctcttctgtcaaccccacacgcctttggcaca |
| (human | | |
| Albumin) | | |
| nSPs | 19 | atggagaaagttcacgttgacatcgaggaagacagcccattcctcagagct |
| | | ttgcagcggagcttcccgcagtttgaggtagaagccaagcaggtcactgat |
| | | aatgaccatgctaatgccagagcgttttcgcatctggcttcaaaactgatc |
| | | gaaacggaggtggacccatccgacacgatccttgacattggaagtgcgccc |
| | | gcccgcagaatgtattctaagcacaagtatcattgtatctgtccgatgaga |
| | | tgtgcggaagatccggacagattgtataagtatgcaactaagctgaagaaa |
| | | aactgtaaggaaataactgataaggaattggacaagaaaatgaaggagctg |
| | | gccgccgtcatgagcgaccctgacctggaaactgagactatgtgcctccac |
| | | gacgacgagtcgtgtcgctacgaagggcaagtcgctgtttaccaggatgta |
| | | tacgcggttgacggaccgacaagtctctatcaccaagccaataagggagtt |
| | | agagtcgcctactggataggctttgacaccaccccttttatgtttaagaac |
| | | ttggctggagcatatccatcatactctaccaactgggccgacgaaaccgtg |
| | | ttaacggctcgtaacataggcctatgcagctctgacgttatggagcggtca |
| | | cgtagagggatgtccattcttagaaagaagtatttgaaaccatccaacaat |
| | | gttctattctctgttggctcgaccatctaccacgagaagagggacttactg |
| | | aggagctggcacctgccgtctgtatttcacttacgtggcaagcaaaattac |
| | | acatgtcggtgtgagactatagttagttgcgacgggtacgtcgttaaaaga |
| | | atagctatcagtccaggcctgtatgggaagccttcaggctatgctgctacg |
| | | atgcaccgcgagggattcttgtgctgcaaagtgacagacacattgaacggg |
| | | gagagggtctcttttcccgtgtgcacgtatgtgccagctacattgtgtgac |
| | | caaatgactggcatactggcaacagatgtcagtgcggacgacgcgcaaaaa |
| | | ctgctggttgggctcaaccagcgtatagtcgtcaacggtcgcacccagaga |
| | | aacaccaataccatgaaaaattaccttttgcccgtagtggcccaggcattt |
| | | gctaggtgggcaaaggaatataaggaagatcaagaagatgaaaggccacta |
| | | ggactacgagatagacagttagtcatggggtgttgttgggcttttagaagg |
| | | cacaagataacatctatttataagcgcccggatacccaaaccatcatcaaa |
| | | gtgaacagcgatttccactcattcgtgctgcccaggataggcagtaacaca |
| | | ttggagatcgggctgagaacaagaatcaggaaaatgttagaggagcacaag |
| | | gagccgtcacctctcattaccgccgaggacgtacaagaagctaagtgcgca |
| | | gccgatgaggctaaggaggtgcgtgaagccgaggagttgcgcgcagctcta |
| | | ccacctttggcagctgatgttgaggagcccactctggaagccgatgtcgac |
| | | ttgatgttacaagaggctggggccggctcagtggagacacctcgtggcttg |
| | | ataaaggttaccagctacgatggcgaggacaagatcggctcttacgctgtg |
| | | ctttctccgcaggctgtactcaagagtgaaaaattatcttgcatccaccct |
| | | ctcgctgaacaagtcatagtgataacacactctggccgaaaagggcgttat |
| | | gccgtggaaccataccatggtaaagtagtggtgccagagggacatgcaata |
| | | cccgtccaggactttcaagctctgagtgaaagtgccaccattgtgtacaac |
| | | gaacgtgagttcgtaaacaggtacctgcaccatattgccacacatggagga |
| | | gcgctgaacactgatgaagaatattacaaaactgtcaagcccagcgagcac |
| | | gacggcgaatacctgtacgacatcgacaggaaacagtgcgtcaagaaagaa |
| | | ctagtcactgggctagggctcacaggcgagctggtggatcctcccttccat |
| | | gaattcgcctacgagagtctgagaacacgaccagccgctccttaccaagta |
| | | ccaaccataggggtgtatggcgtgccaggatcaggcaagtctggcatcatt |
| | | aaaagcgcagtcaccaaaaaagatctagtggtgagcgccaagaaagaaaac |
| | | tgtgcagaaattataagggacgtcaagaaaatgaaagggctggacgtcaat |
| | | gccagaactgtggactcagtgctcttgaatggatgcaaacaccccgtagag |
| | | accctgtatattgacgaagcttttgcttgtcatgcaggtactctcagagcg |
| | | ctcatagccattataagacctaaaaaggcagtgctctgcggggatcccaaa |
| | | cagtgcggtttttttaacatgatgtgcctgaaagtgcattttaaccacgag |
| | | atttgcacacaagtcttccacaaaagcatctctcgccgttgcactaaatct |
| | | gtgacttcggtcgtctcaaccttgttttacgacaaaaaaatgagaacgacg |
| | | aatccgaaagagactaagattgtgattgacactaccggcagtaccaaacct |
| | | aagcaggacgatctcattctcacttgtttcagagggtgggtgaagcagttg |
| | | caaatagattacaaaggcaacgaaataatgacggcagctgcctctcaaggg |
| | | ctgacccgtaaaggtgtgtatgccgttcggtacaaggtgaatgaaaatcct |
| | | ctgtacgcacccacctcagaacatgtgaacgtcctactgacccgcacggag |
| | | gaccgcatcgtgtggaaaacactagccggcgacccatggataaaaacactg |
| | | actgccaagtaccctgggaatttcactgccacgatagaggagtggcaagca |
| | | gagcatgatgccatcatgaggcacatcttggagagaccggaccctaccgac |
| | | gtcttccagaataaggcaaacgtgtgttgggccaaggctttagtgccggtg |
| | | ctgaagaccgctggcatagacatgaccactgaacaatggaacactgtggat |
| | | tattttgaaacggacaaagctcactcagcagagatagtattgaaccaacta |
| | | tgcgtgaggttctttggactcgatctggactccggtctattttctgcaccc |
| | | actgttccgttatccattaggaataatcactgggataactccccgtcgcct |
| | | aacatgtacgggctgaataaagaagtggtccgtcagctctctcgcaggtac |
| | | ccacaactgcctcgggcagttgccactggaagagtctatgacatgaacact |
| | | ggtacactgcgcaattatgatccgcgcataaacctagtacctgtaaacaga |
| | | agactgcctcatgctttagtcctccaccataatgaacacccacagagtgac |
| | | ttttcttcattcgtcagcaaattgaagggcagaactgtcctggtggtcggg |
| | | gaaaagttgtccgtcccaggcaaaatggttgactggttgtcagaccggcct |
| | | gaggctaccttcagagctcggctggatttaggcatcccaggtgatgtgccc |
| | | aaatatgacataatatttgttaatgtgaggaccccatataaataccatcac |
| | | tatcagcagtgtgaagaccatgccattaagcttagcatgttgaccaagaaa |
| | | gcttgtctgcatctgaatcccggcggaacctgtgtcagcataggttatggt |
| | | tacgctgacagggccagcgaaagcatcattggtgctatagcgcggcagttc |
| | | aagttttcccgggtatgcaaaccgaaatcctcacttgaagagacggaagtt |
| | | ctgtttgtattcattgggtacgatcgcaaggcccgtacgcacaattcttac |
| | | aagctttcatcaaccttgaccaacatttatacaggttccagactccacgaa |
| | | gccggatgtgcaccctcatatcatgtggtgcgaggggatattgccacggcc |
| | | accgaaggagtgattataaatgctgctaacagcaaaggacaacctggcgga |
| | | ggggtgtgcggagcgctgtataagaaattcccggaaagcttcgatttacag |
| | | ccgatcgaagtaggaaaagcgcgactggtcaaaggtgcagctaaacatatc |
| | | attcatgccgtaggaccaaacttcaacaaagtttcggaggttgaaggtgac |
| | | aaacagttggcagaggcttatgagtccatcgctaagattgtcaacgataac |
| | | aattacaagtcagtagcgattccactgttgtccaccggcatcttttccggg |
| | | aacaaagatcgactaacccaatcattgaaccatttgctgacagctttagac |
| | | accactgatgcagatgtagccatatactgcagggacaagaaatgggaaatg |
| | | actctcaaggaagcagtggctaggagagaagcagtggaggagatatgcata |
| | | tccgacgactcttcagtgacagaacctgatgcagagctggtgagggtgcat |
| | | ccgaagagttctttggctggaaggaagggctacagcacaagcgatggcaaa |
| | | actttctcatatttggaagggaccaagtttcaccaggcggccaaggatata |
| | | gcagaaattaatgccatgtggcccgttgcaacggaggccaatgagcaggta |
| | | tgcatgtatatcctcggagaaagcatgagcagtattaggtcgaaatgcccc |
| | | gtcgaagagtcggaagcctccacaccacctagcacgctgccttgcttgtgc |
| | | atccatgccatgactccagaaagagtacagcgcctaaaagcctcacgtcca |
| | | gaacaaattactgtgtgctcatcctttccattgccgaagtatagaatcact |
| | | ggtgtgcagaagatccaatgctcccagcctatattgttctcaccgaaagtg |
| | | cctgcgtatattcatccaaggaagtatctcgtggaaacaccaccggtagac |
| | | gagactccggagccatcggcagagaaccaatccacagaggggacacctgaa |
| | | caaccaccacttataaccgaggatgagaccaggactagaacgcctgagccg |
| | | atcatcatcgaagaggaagaagaggatagcataagtttgctgtcagatggc |
| | | ccgacccaccaggtgctgcaagtcgaggcagacattcacgggccgccctct |
| | | gtatctagctcatcctggtccattcctcatgcatccgactttgatgtggac |
| | | agtttatccatacttgacaccctggagggagctagcgtgaccagcggggca |
| | | acgtcagccgagactaactcttacttcgcaaagagtatggagtttctggcg |
| | | cgaccggtgcctgcgcctcgaacagtattcaggaaccctccacatcccgct |
| | | ccgcgcacaagaacaccgtcacttgcacccagcagggcctgctcgagaacc |
| | | agcctagtttccaccccgccaggcgtgaatagggtgatcactagagaggag |
| | | ctcgaggcgcttaccccgtcacgcactcctagcaggtcggtctcgagaacc |
| | | agcctggtctccaacccgccaggcgtaaatagggtgattacaagagaggag |
| | | tttgaggcgttcgtagcacaacaacaatgacggtttgatgcgggtgcatac |
| | | atcttttcctccgacaccggtcaagggcatttacaacaaaaatcagtaagg |
| | | caaacggtgctatccgaagtggtgttggagaggaccgaattggagatttcg |
| | | tatgccccgcgcctcgaccaagaaaaagaagaattactacgcaagaaatta |
| | | cagttaaatcccacacctgctaacagaagcagataccagtccaggaaggtg |
| | | gagaacatgaaagccataacagctagacgtattctgcaaggcctagggcat |
| | | tatttgaaggcagaaggaaaagtggagtgctaccgaaccctgcatcctgtt |
| | | cctttgtattcatctagtgtgaaccgtgccttttcaagccccaaggtcgca |
| | | gtggaagcctgtaacgccatgttgaaagagaactttccgactgtggcttct |
| | | tactgtattattccagagtacgatgcctatttggacatggttgacggagct |
| | | tcatgctgcttagacactgccagtttttgccctgcaaagctgcgcagcttt |
| | | ccaaagaaacactcctatttggaacccacaatacgatcggcagtgccttca |
| | | gcgatccagaacacgctccagaacgtcctggcagctgccacaaaaagaaat |
| | | tgcaatgtcacgcaaatgagagaattgcccgtattggattcggcggccttt |
| | | aatgtggaatgcttcaagaaatatgcgtgtaataatgaatattgggaaacg |
| | | tttaaagaaaaccccatcaggcttactgaagaaaacgtggtaaattacatt |
| | | accaaattaaaaggaccaaaagctgctgctctttttgcgaagacacataat |
| | | ttgaatatgttgcaggacataccaatggacaggtttgtaatggacttaaag |
| | | agagacgtgaaagtgactccaggaacaaaacatactgaagaacggcccaag |
| | | gtacaggtgatccaggctgccgatccgctagcaacagcgtatctgtgcgga |
| | | atccaccgagagctggttaggagattaaatgcggtcctgcttccgaacatt |
| | | catacactgtttgatatgtcggctgaagactttgacgctattatagccgag |
| | | cacttccagcctggggattgtgttctggaaactgacatcgcgtcgtttgat |
| | | aaaagtgaggacgacgccatggctctgaccgcgttaatgattctggaagac |
| | | ttaggtgtggacgcagagctgttgacgctgattgaggcggctttcggcgaa |
| | | atttcatcaatacatttgcccactaaaactaaatttaaattcggagccatg |
| | | atgaaatctggaatgttcctcacactgtttgtgaacacagtcattaacatt |
| | | gtaatcgcaagcagagtgttgagagaacggctaaccggatcaccatgtgca |
| | | gcattcattggagatgacaatatcgtgaaaggagtcaaatcggacaaatta |
| | | atggcagacaggtgcgccacctggttgaatatggaagtcaagattatagat |
| | | gctgtggtgggcgagaaagcgccttatttctgtggagggtttattttgtgt |
| | | gactccgtgaccggcacagcgtgccgtgtggcagaccccctaaaaaggctg |
| | | tttaagcttggcaaacctctggcagcagacgatgaacatgatgatgacagg |
| | | agaagggcattgcatgaagagtcaacacgctggaaccgagtgggtattctt |
| | | tcagagctgtgcaaggcagtagaatcaaggtatgaaaccgtaggaacttcc |
| | | atcatagttatggccatgactactctagctagcagtgttaaatcattcagc |
| | | tacctgagaggggcccctataactctctacggctaacctga |
| Signal Peptide | 20 | atggactggacctggcgaatactgttcttggttgccgccgctacagggact |
| | | cacgca |
| 3′ UTR 1 | 21 | catcgatgatatcgcggccgcatacagcagcaattggcaagctgcttacat |
| | | agaactcgcggcgattggcatgccgccttaaaatttttattttatttttct |
| | | tttcttttccgaatcggattttgtttttaatatttc |
| 3′ UTR 2 | 22 | gctcgctttcttgctgtccaatttctattaaaggttcctttgttccctaag |
| | | tccaactactaaactgggggatattatgaagggccttgagcatctggattc |
| | | tgcctaataaaaaacatttattttcattgcaaagccacaccctggagctag |
| | | c |
| 3′ UTR | 69 | gctgactgtgacttagttgcattacaccctttcttgacaaaacctaacttg |
| (human beta | | cacagaaaacacgatgagattggcatggctttatttgtttttgtttttgtt |
| actin) | | tgtttgtttgttttggcttgactcaggatttaaaaactggaacggtgaagg |
| | | tgacagcagttggttggagcgagcatcccccaaagttctgcaatgtggccg |
| | | agaactttgattgtacattgttctttttttaatggtcattccaaatatcgt |
| | | gagatgcattgttacaggaagtcccttgcctcctaaaagccaccccacttc |
| | | tctctaaggagaatggcccagtcctctcccgagtccacacacgggaggtaa |
| | | tagcattgcttttgggtaaattatataatgcaaaaatcttttaatctttgc |
| | | cttaatacttttttatttttttaattttgaatgatcagcctttgtgactcc |
| | | ccttttttgtcacccaacttgagatgtatgaaggcttttcatctccctcgg |
| | | agtgggtggaggcagccagggcttaactgtacactaacttgagaccagttg |
| | | aataaaagtgcatacca |
| 3′ UTR (human | 70 | catcacatttaaaagcatctcagcctaccatgagaataagagaaagaaaat |
| Albumin for | | gaagatcaaaagcttattcatctgtttttctttttcgttggtgtaaagcca |
| regular | | acaccctgtctaaaaaacataaatttctttaatcattttgcctcttttctc |
| mRNA) | | tgtgcttcaattaataaaaaatggaaagaatctaatagagtggtacagcac |
| | | tgttatttttcaaagatgtgttgctatcctgaaaattctgtaggttctgtg |
| | | gaagttccagtgttctctcttattccacttcggtagaggatttctagtttc |
| | | ttgtgggctaattaaataaatcattaatactcttctaagttatggattata |
| | | aacattcaaaataatattttgacattatgataattctgaataaaagaacaa |
| | | aaacca |
| IL12 | 23 | atgtgggagcttgaaaaagacgtctatgtagtagaagtggactggacacct |
| | | gatgctcctggcgagacagttaacctcacatgcgatacccctgaggaagat |
| | | gatatcacctggacttctgaccagagacacggggtgattgggagcggcaaa |
| | | accctgacgatcactgtgaaggagtttctggacgccggccagtatacctgt |
| | | cacaaggggggggagaccctgagtcatagccacctgttgctgcacaagaag |
| | | gagaatggcatctggtctacagagatcctgaagaactttaagaacaagacc |
| | | ttcctgaagtgtgaagcaccaaactacagtggtcgctttacctgcagctgg |
| | | ctggtccaaagaaacatggacctgaaatttaatataaagagtagttcctcg |
| | | agtcctgattccagggccgtgacgtgcggcatggcaagcctttcagccgaa |
| | | aaagtcacgctggatcagcgagactatgagaagtacagcgttagctgtcag |
| | | gaggacgtaacttgcccgactgccgaggagactctgcccatagagctcgct |
| | | ctggaggccaggcagcagaacaaatatgagaattacagcactagtttcttt |
| | | attagagacatcatcaaacccgacccacccaagaatctgcagatgaagccg |
| | | ctgaagaatagtcaggtcgaggtttcctgggaatatccagattcatggtcc |
| | | actccgcattcttatttttccttaaaattctttgttaggattcagcggaaa |
| | | aaagaaaagatgaaagagacggaggaagggtgcaaccagaagggggccttc |
| | | ctggtggaaaagacaagcactgaggtccaatgtaagggtgggaacgtttgc |
| | | gtgcaggctcaggatcgctactacaacagcagttgctctaagtgggcctgc |
| | | gtaccttgtcgcgtcaggagtggaggggggtcagggggtggctcaggcggc |
| | | ggcagtgggggcagcagggtgatcccagtgtctgggccggcccgttgcttg |
| | | tctcaatccagaaacctcctcaagaccactgacgatatggtaaagactgcc |
| | | cgagagaagctaaaacactactcttgtacagctgaagatatagaccatgag |
| | | gatataacacgggaccagacctctactctgaaaacctgtctgcctcttgag |
| | | ctgcacaagaacgagtcctgtctggctacccgcgaaacctcaagcacaacc |
| | | agaggtagttgcctgcccccacaaaagacatcgcttatgatgaccttgtgt |
| | | ctgggatctatttatgaggacctgaagatgtaccaaactgagttccaggca |
| | | ataaatgctgctctccagaatcacaatcatcaacaaatcatccttgataag |
| | | gggatgctggtcgcaatcgacgagctcatgcaatcgctgaaccacaatggg |
| | | gaaaccctcaggcagaaaccaccggtgggagaggccgacccctaccgtgtt |
| | | aaaatgaagttgtgtattcttttgcatgcattctctacaagagtcgttacc |
| | | atcaatcgcgtcatggggtacctgtcatcagcctag |
| GFP | 24 | atggtgagcaagggcgaggagctgttcaccggggtggtgcccatcctggtc |
| | | gagctggacggcgacgtaaacggccacaagttcagcgtgtccggcgagggc |
| | | gagggcgatgccacctacggcaagctgaccctgaagttcatctgcaccacc |
| | | ggcaagctgcccgtgccctggcccaccctcgtgaccaccctgacctacggc |
| | | gtgcagtgcttcagccgctaccccgaccacatgaagcagcacgacttcttc |
| | | aagtccgccatgcccgaaggctacgtccaggagcgcaccatcttcttcaag |
| | | gacgacggcaactacaagacccgcgccgaggtgaagttcgagggcgacacc |
| | | ctggtgaaccgcatcgagctgaagggcatcgacttcaaggaggacggcaac |
| | | atcctggggcacaagctggagtacaactacaacagccacaacgtctatatc |
| | | atggccgacaagcagaagaacggcatcaaggtgaacttcaagatccgccac |
| | | aacatcgaggacggcagcgtgcagctcgccgaccactaccagcagaacacc |
| | | cccatcggcgacggccccgtgctgctgcccgacaaccactacctgagcacc |
| | | cagtccgccctgagcaaagaccccaacgagaagcgcgatcacatggtcctg |
| | | ctggagttcgtgaccgccgccgggatcactctcggcatggacgagctgtac |
| | | aagtag |
| Luciferase | 25 | atggaggacgctaaaaatatcaagaagggtcccgctcctttctatccactc |
| | | gaggacggtaccgctggtgagcaactgcacaaggctatgaaacggtatgct |
| | | cttgtgccaggcaccatagctttcaccgacgcccacatagaggtggacatt |
| | | acttacgcagagtactttgagatgtctgtccggctggcagaggcaatgaaa |
| | | agatatggcctcaacacaaaccataggatagtggtttgcagcgagaattct |
| | | cttcagttttttatgcccgtcctcggggctcttttcatcggcgtggctgtc |
| | | gcccccgccaacgacatatacaacgagcgagagctgctgaacagcatggga |
| | | atcagtcagcctacagtggtttttgtatcaaagaagggcttgcaaaagatc |
| | | cttaacgtacagaaaaagctccctattattcagaagatcattataatggat |
| | | tcaaagaccgactatcagggattccagtcaatgtatactttcgtcacttcc |
| | | caccttcctcctgggtttaatgaatacgacttcgttcccgaaagtttcgat |
| | | cgggacaaaactattgcactcatcatgaactcctctgggtccactgggctg |
| | | cctaaaggtgtcgcacttccccacaggactgcatgtgtaaggttctctcac |
| | | gcacgagatcccatattcggtaaccaaatcattcctgataccgctatcctc |
| | | tctgtagtgcctttccaccatggattcggcatgttcaccactctgggttac |
| | | ttgatatgtgggttccgcgtggtccttatgtacagatttgaagaggagctc |
| | | tttttgaggagcttgcaggactacaaaatccaaagtgctctcctcgtcccc |
| | | acattgttctcattcttcgccaaatctactctcatcgacaagtacgatttg |
| | | agcaacttgcacgaaattgcttctgggggggctccactctccaaagaagtc |
| | | ggcgaagccgttgctaaaaggttccacttgccaggcattcgccaggggtat |
| | | ggccttactgagacaacctcagctattctcattacccctgagggtgacgat |
| | | aagcctggtgccgtcggtaaggtagtaccatttttcgaggcaaaggtggtc |
| | | gacttggataccggaaaaaccctcggtgtgaatcagagaggcgaattgtgt |
| | | gtcaggggtccaatgatcatgagcggttacgtcaataatcccgaggccact |
| | | aacgccttgattgataaagatggttggcttcattcaggtgatatagcatac |
| | | tgggatgaggatgaacatttcttcattgtcgataggctcaaaagcctgatc |
| | | aagtataagggttaccaagtggcccctgccgaactcgaatctatactgctg |
| | | caacatccaaacatttttgacgctggcgtggccggactgcccgatgatgat |
| | | gctggtgaactccctgccgccgttgtcgtcctggaacatggtaagaccatg |
| | | acagaaaaggagattgtagattacgtggcttcacaagtgactacagctaag |
| | | aaattgcgcgggggtgtcgttttcgtggacgaggtacccaagggcctcact |
| | | gggaagctcgacgccagaaaaatccgagaaatcctgattaaagcaaagaag |
| | | ggcggaaagattgcagtgtag |
| VEE Structural | 26 | atgttcccgttccagccaatgtatccgatgcagccaatgccctatcgcaac |
| Proteins | | ccgttcgcggccccgcgcaggccctggttccccagaaccgacccttttctg |
| | | gcgatgcaggtgcaggaattaacccgctcgatggctaacctgacgttcaag |
| | | caacgccgggacgcgccacctgaggggccatccgctaagaaaccgaagaag |
| | | gaggcctcgcaaaaacagaaagggggaggccaagggaagaagaagaagaac |
| | | caagggaagaagaaggctaagacagggccgcctaatccgaaggcacagaat |
| | | ggaaacaagaagaagaccaacaagaaaccaggcaagagacagcgcatggtc |
| | | atgaaattggaatctgacaagacgttcccaatcatgttggaagggaagata |
| | | aacggctacgcttgtgtggtcggagggaagttattcaggccgatgcatgtg |
| | | gaaggcaagatcgacaacgacgttctggccgcgcttaagacgaagaaagca |
| | | tccaaatacgatcttgagtatgcagatgtgccacagaacatgcgggccgat |
| | | acattcaaatacacccatgagaaaccccaaggctattacagctggcatcat |
| | | ggagcagtccaatatgaaaatgggcgtttcacggtgccgaaaggagttggg |
| | | gccaagggagacagcggacgacccattctggataaccagggacgggtggtc |
| | | gctattgtgctgggaggtgtgaatgaaggatctaggacagccctttcagtc |
| | | gtcatgtggaacgagaagggagttaccgtgaagtatactccggagaactgc |
| | | gagcaatggtcactagtgaccaccatgtgtctgctcgccaatgtgacgttc |
| | | ccatgtgctcaaccaccaatttgctacgacagaaaaccagcagagactttg |
| | | gccatgctcagcgttaacgttgacaacccgggctacgatgagctgctggaa |
| | | gcagctgttaagtgccccggaaggaaaaggagatccaccgaggagctgttt |
| | | aatgagtataagctaacgcgcccttacatggccagatgcatcagatgtgca |
| | | gttgggagctgccatagtccaatagcaatcgaggcagtaaagagcgacggg |
| | | cacgacggttatgttagacttcagacttcctcgcagtatggcctggattcc |
| | | tccggcaacttaaagggcaggaccatgcggtatgacatgcacgggaccatt |
| | | aaagagataccactacatcaagtgtcactctatacatctcgcccgtgtcac |
| | | attgtggatgggcacggttatttcctgcttgccaggtgcccggcaggggac |
| | | tccatcaccatggaatttaagaaagattccgtcagacactcctgctcggtg |
| | | ccgtatgaagtgaaatttaatcctgtaggcagagaactctatactcatccc |
| | | ccagaacacggagtagagcaagcgtgccaagtctacgcacatgatgcacag |
| | | aacagaggagcttatgtcgagatgcacctcccgggctcagaagtggacagc |
| | | agtttggtttccttgagcggcagttcagtcaccgtgacacctcctgatggg |
| | | actagcgccctggtggaatgcgagtgtggcggcacaaagatctccgagacc |
| | | atcaacaagacaaaacagttcagccagtgcacaaagaaggagcagtgcaga |
| | | gcatatcggctgcagaacgataagtgggtgtataattctgacaaactgccc |
| | | aaagcagcgggagccaccttaaaaggaaaactgcatgtcccattcttgctg |
| | | gcagacggcaaatgcaccgtgcctctagcaccagaacctatgataaccttc |
| | | ggtttcagatcagtgtcactgaaactgcaccctaagaatcccacatatcta |
| | | atcacccgccaacttgctgatgagcctcactacacgcacgagctcatatct |
| | | gaaccagctgttaggaattttaccgtcaccgaaaaagggtgggagtttgta |
| | | tggggaaaccacccgccgaaaaggttttgggcacaggaaacagcacccgga |
| | | aatccacatgggctaccgcacgaggtgataactcattattaccacagatac |
| | | cctatgtccaccatcctgggtttgtcaatttgtgccgccattgcaaccgtt |
| | | tccgttgcagcgtctacctggctgttttgcagatctagagttgcgtgccta |
| | | actccttaccggctaacacctaacgctaggataccattttgtctggctgtg |
| | | ctttgctgcgcccgcactgcccgggccgagaccacctgggagtccttggat |
| | | cacctatggaacaataaccaacagatgttctggattcaattgctgatccct |
| | | ctggccgccttgatcgtagtgactcgcctgctcaggtgcgtgtgctgtgtc |
| | | gtgccttttttagtcatggccggcgccgcaggcgccggcgcctacgagcac |
| | | gcgaccacgatgccgagccaagcgggaatctcgtataacactatagtcaac |
| | | agagcaggctacgcaccactccctatcagcataacaccaacaaagatcaag |
| | | ctgatacctacagtgaacttggagtacgtcacctgccactacaaaacagga |
| | | atggattcaccagccatcaaatgctgcggatctcaggaatgcactccaact |
| | | tacaggcctgatgaacagtgcaaagtcttcacaggggtttacccgttcatg |
| | | tggggtggtgcatattgcttttgcgacactgagaacacccaagtcagcaag |
| | | gcctacgtaatgaaatctgacgactgccttgcggatcatgctgaagcatat |
| | | aaagcgcacacagcctcagtgcaggcgttcctcaacatcacagtgggagaa |
| | | cactctattgtgactaccgtgtatgtgaatggagaaactcctgtgaatttc |
| | | aatggggtcaaaataactgcaggtccgctttccacagcttggacacccttt |
| | | gatcgcaaaatcgtgcagtatgccggggagatctataattatgattttcct |
| | | gagtatggggcaggacaaccaggagcatttggagatatacaatccagaaca |
| | | gtctcaagctctgatctgtatgccaataccaacctagtgctgcagagaccc |
| | | aaagcaggagcgatccacgtgccatacactcaggcaccttcgggttttgag |
| | | caatggaagaaagataaagctccatcattgaaatttaccgcccctttcgga |
| | | tgcgaaatatatacaaaccccattcgcgccgaaaactgtgctgtagggtca |
| | | attccattagcctttgacattcccgacgccttgttcaccagggtgtcagaa |
| | | acaccgacactttcagcggccgaatgcactcttaacgagtgcgtgtattct |
| | | tccgactttggtgggatcgccacggtcaagtactcggccagcaagtcaggc |
| | | aagtgcgcagtccatgtgccatcagggactgctaccctaaaagaagcagca |
| | | gtcgagctaaccgagcaagggtcggcgactatccatttctcgaccgcaaat |
| | | atccacccggagttcaggctccaaatatgcacatcatatgttacgtgcaaa |
| | | ggtgattgtcaccccccgaaagaccatattgtgacacaccctcagtatcac |
| | | gcccaaacatttacagccgcggtgtcaaaaaccgcgtggacgtggttaaca |
| | | tccctgctgggaggatcagccgtaattattataattggcttggtgctggct |
| | | actattgtggccatgtacgtgctgaccaaccagaaacataattga |
| Linker | 27 | cgcgtgataacgcaggaaagaacatgtgagcaaaaggccagcaaaaggcca |
| | | ggaaccgtaaaaaggccgcgttgctggcgtt |
| | 28 | cacatttccccgaaaagtgccacctgagctc |
| | 29 | ttcgaaggcgcgcctctagagccacc |
| | 30 | catcgatgatatcgcggccgcatacagcagc |
| | 31 | ggaggggggtcagggggtggctcaggcggcggcagtgggggcagc |
| | 32 | ggcggtagtggtggtgggagc |
| | 33 | ggtggcggaagcggagggggcagcggaggtgggagcggagggagc |
| | 34 | ggagggtcaggaggaggatcc |
| | 35 | gaagatcctttgatcttttctacggggtctgacgctcagtggaacgaaaac |
| | | tcacgttaagggattttggtcatgagattatcaaaaaggatcttcacctag |
| | | atccttttaaattaaaaatgaagttttaaatcaatctaaagtatat |
| | 36 | cgcggaagagc |
| | 37 | cgcgtatcgatgatatcgcggccgcatacagcagcaattggcaagctgctt |
| | | acatagaactcgcggcgattggcatgccgccttaaaatttttattttattt |
| | | ttcttttcttttccgaatcggattttgtttttaatatttcaaaaaaaaaaa |
| | | aaaaaaaaaaaaaaacgcgtgataacgcaggaaagaacatgtgagcaaaag |
| | | gccagcaaaaggccaggaaccgtaaaaaggccgcgttgctggcgtt |
| Nucleic Acid | 38 | cttgatgttacaagaggctggggccggctcagtggagacacctcgtggctt |
| Template, | | gataaaggttaccagctacgatggcgaggacaagatcggctcttacgctgt |
| Sam002 | | gctttctccgcaggctgtactcaagagtgaaaaattatcttgcatccaccc |
| encoding IL12 | | tctcgctgaacaagtcatagtgataacacactctggccgaaaagggcgtta |
| | | tgccgtggaaccataccatggtaaagtagtggtgccagagggacatgcaat |
| | | acccgtccaggactttcaagctctgagtgaaagtgccaccattgtgtacaa |
| | | cgaacgtgagttcgtaaacaggtacctgcaccatattgccacacatggagg |
| | | agcgctgaacactgatgaagaatattacaaaactgtcaagcccagcgagca |
| | | cgacggcgaatacctgtacgacatcgacaggaaacagtgcgtcaagaaaga |
| | | actagtcactgggctagggctcacaggcgagctggtggatcctcccttcca |
| | | tgaattcgcctacgagagtctgagaacacgaccagccgctccttaccaagt |
| | | accaaccataggggtgtatggcgtgccaggatcaggcaagtctggcatcat |
| | | taaaagcgcagtcaccaaaaaagatctagtggtgagcgccaagaaagaaaa |
| | | ctgtgcagaaattataagggacgtcaagaaaatgaaagggctggacgtcaa |
| | | tgccagaactgtggactcagtgctcttgaatggatgcaaacaccccgtaga |
| | | gaccctgtatattgacgaagcttttgcttgtcatgcaggtactctcagagc |
| | | gctcatagccattataagacctaaaaaggcagtgctctgcggggatcccaa |
| | | acagtgcggtttttttaacatgatgtgcctgaaagtgcattttaaccacga |
| | | gatttgcacacaagtcttccacaaaagcatctctcgccgttgcactaaatc |
| | | tgtgacttcggtcgtctcaaccttgttttacgacaaaaaaatgagaacgac |
| | | gaatccgaaagagactaagattgtgattgacactaccggcagtaccaaacc |
| | | taagcaggacgatctcattctcacttgtttcagagggtgggtgaagcagtt |
| | | gcaaatagattacaaaggcaacgaaataatgacggcagctgcctctcaagg |
| | | gctgacccgtaaaggtgtgtatgccgttcggtacaaggtgaatgaaaatcc |
| | | tctgtacgcacccacctcagaacatgtgaacgtcctactgacccgcacgga |
| | | ggaccgcatcgtgtggaaaacactagccggcgacccatggataaaaacact |
| | | gactgccaagtaccctgggaatttcactgccacgatagaggagtggcaagc |
| | | agagcatgatgccatcatgaggcacatcttggagagaccggaccctaccga |
| | | cgtcttccagaataaggcaaacgtgtgttgggccaaggctttagtgccggt |
| | | gctgaagaccgctggcatagacatgaccactgaacaatggaacactgtgga |
| | | ttattttgaaacggacaaagctcactcagcagagatagtattgaaccaact |
| | | atgcgtgaggttctttggactcgatctggactccggtctattttctgcacc |
| | | cactgttccgttatccattaggaataatcactgggataactccccgtcgcc |
| | | taacatgtacgggctgaataaagaagtggtccgtcagctctctcgcaggta |
| | | cccacaactgcctcgggcagttgccactggaagagtctatgacatgaacac |
| | | tggtacactgcgcaattatgatccgcgcataaacctagtacctgtaaacag |
| | | aagactgcctcatgctttagtcctccaccataatgaacacccacagagtga |
| | | cttttcttcattcgtcagcaaattgaagggcagaactgtcctggtggtcgg |
| | | ggaaaagttgtccgtcccaggcaaaatggttgactggttgtcagaccggcc |
| | | tgaggctaccttcagagctcggctggatttaggcatcccaggtgatgtgcc |
| | | caaatatgacataatatttgttaatgtgaggaccccatataaataccatca |
| | | ctatcagcagtgtgaagaccatgccattaagcttagcatgttgaccaagaa |
| | | agcttgtctgcatctgaatcccggcggaacctgtgtcagcataggttatgg |
| | | ttacgctgacagggccagcgaaagcatcattggtgctatagcgcggcagtt |
| | | caagttttcccgggtatgcaaaccgaaatcctcacttgaagagacggaagt |
| | | tctgtttgtattcattgggtacgatcgcaaggcccgtacgcacaattctta |
| | | caagctttcatcaaccttgaccaacatttatacaggttccagactccacga |
| | | agccggatgtgcaccctcatatcatgtggtgcgaggggatattgccacggc |
| | | caccgaaggagtgattataaatgctgctaacagcaaaggacaacctggcgg |
| | | aggggtgtgcggagcgctgtataagaaattcccggaaagcttcgatttaca |
| | | gccgatcgaagtaggaaaagcgcgactggtcaaaggtgcagctaaacatat |
| | | cattcatgccgtaggaccaaacttcaacaaagtttcggaggttgaaggtga |
| | | caaacagttggcagaggcttatgagtccatcgctaagattgtcaacgataa |
| | | caattacaagtcagtagcgattccactgttgtccaccggcatcttttccgg |
| | | gaacaaagatcgactaacccaatcattgaaccatttgctgacagctttaga |
| | | caccactgatgcagatgtagccatatactgcagggacaagaaatgggaaat |
| | | gactctcaaggaagcagtggctaggagagaagcagtggaggagatatgcat |
| | | atccgacgactcttcagtgacagaacctgatgcagagctggtgagggtgca |
| | | tccgaagagttctttggctggaaggaagggctacagcacaagcgatggcaa |
| | | aactttctcatatttggaagggaccaagtttcaccaggcggccaaggatat |
| | | agcagaaattaatgccatgtggcccgttgcaacggaggccaatgagcaggt |
| | | atgcatgtatatcctcggagaaagcatgagcagtattaggtcgaaatgccc |
| | | cgtcgaagagtcggaagcctccacaccacctagcacgctgccttgcttgtg |
| | | catccatgccatgactccagaaagagtacagcgcctaaaagcctcacgtcc |
| | | agaacaaattactgtgtgctcatcctttccattgccgaagtatagaatcac |
| | | tggtgtgcagaagatccaatgctcccagcctatattgttctcaccgaaagt |
| | | gcctgcgtatattcatccaaggaagtatctcgtggaaacaccaccggtaga |
| | | cgagactccggagccatcggcagagaaccaatccacagaggggacacctga |
| | | acaaccaccacttataaccgaggatgagaccaggactagaacgcctgagcc |
| | | gatcatcatcgaagaggaagaagaggatagcataagtttgctgtcagatgg |
| | | cccgacccaccaggtgctgcaagtcgaggcagacattcacgggccgccctc |
| | | tgtatctagctcatcctggtccattcctcatgcatccgactttgatgtgga |
| | | cagtttatccatacttgacaccctggagggagctagcgtgaccagcggggc |
| | | aacgtcagccgagactaactcttacttcgcaaagagtatggagtttctggc |
| | | gcgaccggtgcctgcgcctcgaacagtattcaggaaccctccacatcccgc |
| | | tccgcgcacaagaacaccgtcacttgcacccagcagggcctgctcgagaac |
| | | cagcctagtttccaccccgccaggcgtgaatagggtgatcactagagagga |
| | | gctcgaggcgcttaccccgtcacgcactcctagcaggtcggtctcgagaac |
| | | cagcctggtctccaacccgccaggcgtaaatagggtgattacaagagagga |
| | | gtttgaggcgttcgtagcacaacaacaatgacggtttgatgcgggtgcata |
| | | catcttttcctccgacaccggtcaagggcatttacaacaaaaatcagtaag |
| | | gcaaacggtgctatccgaagtggtgttggagaggaccgaattggagatttc |
| | | gtatgccccgcgcctcgaccaagaaaaagaagaattactacgcaagaaatt |
| | | acagttaaatcccacacctgctaacagaagcagataccagtccaggaaggt |
| | | ggagaacatgaaagccataacagctagacgtattctgcaaggcctagggca |
| | | ttatttgaaggcagaaggaaaagtggagtgctaccgaaccctgcatcctgt |
| | | tcctttgtattcatctagtgtgaaccgtgccttttcaagccccaaggtcgc |
| | | agtggaagcctgtaacgccatgttgaaagagaactttccgactgtggcttc |
| | | ttactgtattattccagagtacgatgcctatttggacatggttgacggagc |
| | | ttcatgctgcttagacactgccagtttttgccctgcaaagctgcgcagctt |
| | | tccaaagaaacactcctatttggaacccacaatacgatcggcagtgccttc |
| | | agcgatccagaacacgctccagaacgtcctggcagctgccacaaaaagaaa |
| | | ttgcaatgtcacgcaaatgagagaattgcccgtattggattcggcggcctt |
| | | taatgtggaatgcttcaagaaatatgcgtgtaataatgaatattgggaaac |
| | | gtttaaagaaaaccccatcaggcttactgaagaaaacgtggtaaattacat |
| | | taccaaattaaaaggaccaaaagctgctgctctttttgcgaagacacataa |
| | | tttgaatatgttgcaggacataccaatggacaggtttgtaatggacttaaa |
| | | gagagacgtgaaagtgactccaggaacaaaacatactgaagaacggcccaa |
| | | ggtacaggtgatccaggctgccgatccgctagcaacagcgtatctgtgcgg |
| | | aatccaccgagagctggttaggagattaaatgcggtcctgcttccgaacat |
| | | tcatacactgtttgatatgtcggctgaagactttgacgctattatagccga |
| | | gcacttccagcctggggattgtgttctggaaactgacatcgcgtcgtttga |
| | | taaaagtgaggacgacgccatggctctgaccgcgttaatgattctggaaga |
| | | cttaggtgtggacgcagagctgttgacgctgattgaggcggctttcggcga |
| | | aatttcatcaatacatttgcccactaaaactaaatttaaattcggagccat |
| | | gatgaaatctggaatgttcctcacactgtttgtgaacacagtcattaacat |
| | | tgtaatcgcaagcagagtgttgagagaacggctaaccggatcaccatgtgc |
| | | agcattcattggagatgacaatatcgtgaaaggagtcaaatcggacaaatt |
| | | aatggcagacaggtgcgccacctggttgaatatggaagtcaagattataga |
| | | tgctgtggtgggcgagaaagcgccttatttctgtggagggtttattttgtg |
| | | tgactccgtgaccggcacagcgtgccgtgtggcagaccccctaaaaaggct |
| | | gtttaagcttggcaaacctctggcagcagacgatgaacatgatgatgacag |
| | | gagaagggcattgcatgaagagtcaacacgctggaaccgagtgggtattct |
| | | ttcagagctgtgcaaggcagtagaatcaaggtatgaaaccgtaggaacttc |
| | | catcatagttatggccatgactactctagctagcagtgttaaatcattcag |
| | | ctacctgagaggggcccctataactctctacggctaacctgaatggactac |
| | | gacatagtctagtccgccaagttcgaaggcgcgcctctagagccaccatgg |
| | | actggacctggcgaatactgttcttggttgccgccgctacagggactcacg |
| | | caatgtgggagcttgaaaaagacgtctatgtagtagaagtggactggacac |
| | | ctgatgctcctggcgagacagttaacctcacatgcgatacccctgaggaag |
| | | atgatatcacctggacttctgaccagagacacggggtgattgggagcggca |
| | | aaaccctgacgatcactgtgaaggagtttctggacgccggccagtatacct |
| | | gtcacaaggggggggagaccctgagtcatagccacctgttgctgcacaaga |
| | | aggagaatggcatctggtctacagagatcctgaagaactttaagaacaaga |
| | | ccttcctgaagtgtgaagcaccaaactacagtggtcgctttacctgcagct |
| | | ggctggtccaaagaaacatggacctgaaatttaatataaagagtagttcct |
| | | cgagtcctgattccagggccgtgacgtgcggcatggcaagcctttcagccg |
| | | aaaaagtcacgctggatcagcgagactatgagaagtacagcgttagctgtc |
| | | aggaggacgtaacttgcccgactgccgaggagactctgcccatagagctcg |
| | | ctctggaggccaggcagcagaacaaatatgagaattacagcactagtttct |
| | | ttattagagacatcatcaaacccgacccacccaagaatctgcagatgaagc |
| | | cgctgaagaatagtcaggtcgaggtttcctgggaatatccagattcatggt |
| | | ccactccgcattcttatttttccttaaaattctttgttaggattcagcgga |
| | | aaaaagaaaagatgaaagagacggaggaagggtgcaaccagaagggggcct |
| | | tcctggtggaaaagacaagcactgaggtccaatgtaagggtgggaacgttt |
| | | gcgtgcaggctcaggatcgctactacaacagcagttgctctaagtgggcct |
| | | gcgtaccttgtcgcgtcaggagtggaggggggtcagggggtggctcaggcg |
| | | gcggcagtgggggcagcagggtgatcccagtgtctgggccggcccgttgct |
| | | tgtctcaatccagaaacctcctcaagaccactgacgatatggtaaagactg |
| | | cccgagagaagctaaaacactactcttgtacagctgaagatatagaccatg |
| | | aggatataacacgggaccagacctctactctgaaaacctgtctgcctcttg |
| | | agctgcacaagaacgagtcctgtctggctacccgcgaaacctcaagcacaa |
| | | ccagaggtagttgcctgcccccacaaaagacatcgcttatgatgaccttgt |
| | | gtctgggatctatttatgaggacctgaagatgtaccaaactgagttccagg |
| | | caataaatgctgctctccagaatcacaatcatcaacaaatcatccttgata |
| | | aggggatgctggtcgcaatcgacgagctcatgcaatcgctgaaccacaatg |
| | | gggaaaccctcaggcagaaaccaccggtgggagaggccgacccctaccgtg |
| | | ttaaaatgaagttgtgtattcttttgcatgcattctctacaagagtcgtta |
| | | ccatcaatcgcgtcatggggtacctgtcatcagcctagcatcgatgatatc |
| | | gcggccgcatacagcagcaattggcaagctgcttacatagaactcgcggcg |
| | | attggcatgccgccttaaaatttttattttatttttcttttcttttccgaa |
| | | tcggattttgtttttaatatttcaaaaaaaaaaaaaaaaaaaaaaaaaacg |
| | | cggaagagccgcgtgataacgcaggaaagaacatgtgagcaaaaggccagc |
| | | aaaaggccaggaaccgtaaaaaggccgcgttgctggcgtttttccataggc |
| | | tccgcccccctgacgagcatcacaaaaatcgacgctcaagtcagaggtggc |
| | | gaaacccgacaggactataaagataccaggcgtttccccctggaagctccc |
| | | tcgtgcgctctcctgttccgaccctgccgcttaccggatacctgtccgcct |
| | | ttctcccttcgggaagcgtggcgctttctcatagctcacgctgtaggtatc |
| | | tcagttcggtgtaggtcgttcgctccaagctgggctgtgtgcacgaacccc |
| | | ccgttcagcccgaccgctgcgccttatccggtaactatcgtcttgagtcca |
| | | acccggtaagacacgacttatcgccactggcagcagccactggtaacagga |
| | | ttagcagagcgaggtatgtaggcggtgctacagagttcttgaagtggtggc |
| | | ctaactacggctacactagaagaacagtatttggtatctgcgctctgctga |
| | | agccagttaccttcggaaaaagagttggtagctcttgatccggcaaacaaa |
| | | ccaccgctggtagcggtggtttttttgtttgcaagcagcagattacgcgca |
| | | gaaaaaaaggatctcaagaagatcctttgatcttttctacggggtctgacg |
| | | ctcagtggaacgaaaactcacgttaagggattttggtcatgagattatcaa |
| | | aaaggatcttcacctagatccttttaaattaaaaatgaagttttaaatcaa |
| | | tctaaagtatatatgagtaacctgaggctatggcagggcctgccgccccga |
| | | cgttggctgcgagccctgggccttcacccgaacttggggggtggggtgggg |
| | | aaaaggaagaaacgcgggcgtattggccccaatggggtctcggtggggtat |
| | | cgacagagtgccagccctgggaccgaaccccgcgtttatgaacaaacgacc |
| | | caacaccgtgcgttttattctgtctttttattgccgtcatagcgcgggttc |
| | | cttccggtattgtctccttccgtgtttcagttagcctccccctagggtggg |
| | | cgaagaactccagcatgagatccccgcgctggaggatcatccagccggcgt |
| | | cccggaaaacgattccgaagcccaacctttcatagaaggcggcggtggaat |
| | | cgaaatctcgtgatggcaggttgggcgtcgcttggtcggtcatttcgaacc |
| | | ccagagtcccgctcagaagaactcgtcaagaaggcgatagaaggcgatgcg |
| | | ctgcgaatcgggagcggcgataccgtaaagcacgaggaagcggtcagccca |
| | | ttcgccgccaagctcttcagcaatatcacgggtagccaacgctatgtcctg |
| | | atagcgatccgccacacccagccggccacagtcgatgaatccagaaaagcg |
| | | gccattttccaccatgatattcggcaagcaggcatcgccatgggtcacgac |
| | | gagatcctcgccgtcgggcatgctcgccttgagcctggcgaacagttcggc |
| | | tggcgcgagcccctgatgctcttcgtccagatcatcctgatcgacaagacc |
| | | ggcttccatccgagtacgtgctcgctcgatgcgatgtttcgcttggtggtc |
| | | gaatgggcaggtagccggatcaagcgtatgcagccgccgcattgcatcagc |
| | | catgatggatactttctcggcaggagcaaggtgagatgacaggagatcctg |
| | | ccccggcacttcgcccaatagcagccagtcccttcccgcttcagtgacaac |
| | | gtcgagcacagctgcgcaaggaacgcccgtcgtggccagccacgatagccg |
| | | cgctgcctcgtcttgcagttcattcagggcaccggacaggtcggtcttgac |
| | | aaaaagaaccgggcgcccctgcgctgacagccggaacacggcggcatcaga |
| | | gcagccgattgtctgttgtgcccagtcatagccgaatagcctctccaccca |
| | | agcggccggagaacctgcgtgcaatccatcttgttcaatcatgcgaaacga |
| | | tcctcatcctgtctcttgatcgatctttgcaaaagcctaggcctccaaaaa |
| | | agcctcctcactacttctggaatagctcagaggccgaggcggcctcggcct |
| | | ctgcataaataaaaaaaattagtcagccatggggcggagaatgggcggaac |
| | | tgggcggagttaggggcgggatgggcggagttaggggcgggactatggttg |
| | | ctgactaattgagatgcatgctttgcatacttctgcctgctggggagcctg |
| | | gggactttccacacctggttgctgactaattgagatgcatgctttgcatac |
| | | ttctgcctgctggggagcctggggactttccacaccctaactgacacacat |
| | | tccacagctggttctttccgcctcaggactcttcctttttcaatattattg |
| | | aagcatttatcagggttattgtctcatgagcggatacatatttgaatgtat |
| | | ttagaaaaataaacaaataggggttccgcgcacatttccccgaaaagtgcc |
| | | acctgagctctaatacgactcactataggataggcggcgcatgagagaagc |
| | | ccagaccaattacctacccaaaatggagaaagttcacgttgacatcgagga |
| | | agacagcccattcctcagagctttgcagcggagcttcccgcagtttgaggt |
| | | agaagccaagcaggtcactgataatgaccatgctaatgccagagcgttttc |
| | | gcatctggcttcaaaactgatcgaaacggaggtggacccatccgacacgat |
| | | ccttgacattggaagtgcgcccgcccgcagaatgtattctaagcacaagta |
| | | tcattgtatctgtccgatgagatgtgcggaagatccggacagattgtataa |
| | | gtatgcaactaagctgaagaaaaactgtaaggaaataactgataaggaatt |
| | | ggacaagaaaatgaaggagctggccgccgtcatgagcgaccctgacctgga |
| | | aactgagactatgtgcctccacgacgacgagtcgtgtcgctacgaagggca |
| | | agtcgctgtttaccaggatgtatacgcggttgacggaccgacaagtctcta |
| | | tcaccaagccaataagggagttagagtcgcctactggataggctttgacac |
| | | caccccttttatgtttaagaacttggctggagcatatccatcatactctac |
| | | caactgggccgacgaaaccgtgttaacggctcgtaacataggcctatgcag |
| | | ctctgacgttatggagcggtcacgtagagggatgtccattcttagaaagaa |
| | | gtatttgaaaccatccaacaatgttctattctctgttggctcgaccatcta |
| | | ccacgagaagagggacttactgaggagctggcacctgccgtctgtatttca |
| | | cttacgtggcaagcaaaattacacatgtcggtgtgagactatagttagttg |
| | | cgacgggtacgtcgttaaaagaatagctatcagtccaggcctgtatgggaa |
| | | gccttcaggctatgctgctacgatgcaccgcgagggattcttgtgctgcaa |
| | | agtgacagacacattgaacggggagagggtctcttttcccgtgtgcacgta |
| | | tgtgccagctacattgtgtgaccaaatgactggcatactggcaacagatgt |
| | | cagtgcggacgacgcgcaaaaactgctggttgggctcaaccagcgtatagt |
| | | cgtcaacggtcgcacccagagaaacaccaataccatgaaaaattacctttt |
| | | gcccgtagtggcccaggcatttgctaggtgggcaaaggaatataaggaaga |
| | | tcaagaagatgaaaggccactaggactacgagatagacagttagtcatggg |
| | | gtgttgttgggcttttagaaggcacaagataacatctatttataagcgccc |
| | | ggatacccaaaccatcatcaaagtgaacagcgatttccactcattcgtgct |
| | | gcccaggataggcagtaacacattggagatcgggctgagaacaagaatcag |
| | | gaaaatgttagaggagcacaaggagccgtcacctctcattaccgccgagga |
| | | cgtacaagaagctaagtgcgcagccgatgaggctaaggaggtgcgtgaagc |
| | | cgaggagttgcgcgcagctctaccacctttggcagctgatgttgaggagcc |
| | | cactctggaagccgatgtcga |
| Nucleic Acid | 39 | gctcgctttcttgctgtccaatttctattaaaggttcctttgttccctaag |
| Template, | | tccaactactaaactgggggatattatgaagggccttgagcatctggattc |
| mRNA | | tgcctaataaaaaacatttattttcattgcaaagccacaccctggagctag |
| encoding | | caaaaaaaaaaaaaaaaaaaaaaaaaaaaaaaaaaaaaaaaaaaaaaaacg |
| luciferase | | cgtatcgatgatatcgcggccgcatacagcagcaattggcaagctgcttac |
| | | atagaactcgcggcgattggcatgccgccttaaaatttttattttattttt |
| | | cttttcttttccgaatcggattttgtttttaatatttcaaaaaaaaaaaaa |
| | | aaaaaaaaaaaaacgcgtgataacgcaggaaagaacatgtgagcaaaaggc |
| | | cagcaaaaggccaggaaccgtaaaaaggccgcgttgctggcgtttttccat |
| | | aggctccgcccccctgacgagcatcacaaaaatcgacgctcaagtcagagg |
| | | tggcgaaacccgacaggactataaagataccaggcgtttccccctggaagc |
| | | tccctcgtgcgctctcctgttccgaccctgccgcttaccggatacctgtcc |
| | | gcctttctcccttcgggaagcgtggcgctttctcatagctcacgctgtagg |
| | | tatctcagttcggtgtaggtcgttcgctccaagctgggctgtgtgcacgaa |
| | | ccccccgttcagcccgaccgctgcgccttatccggtaactatcgtcttgag |
| | | tccaacccggtaagacacgacttatcgccactggcagcagccactggtaac |
| | | aggattagcagagcgaggtatgtaggcggtgctacagagttcttgaagtgg |
| | | tggcctaactacggctacactagaagaacagtatttggtatctgcgctctg |
| | | ctgaagccagttaccttcggaaaaagagttggtagctcttgatccggcaaa |
| | | caaaccaccgctggtagcggtttttttgtttgcaagcagcagattacgcgc |
| | | agaaaaaaaggatctcaagaagatcctttgatcttttctacggggtctgac |
| | | gctcagtggaacgaaaactcacgttaagggattttggtcatgagattatca |
| | | aaaaggatcttcacctagatccttttaaattaaaaatgaagttttaaatca |
| | | atctaaagtatatatgagtaaacttggtctgacagttaccaatgcttaatc |
| | | agtgaggcacctatctcagcgatctgtctatttcgttcatccatagttgcc |
| | | tgactccccgtcgtgtagataactacgatacgggagggcttaccatctggc |
| | | cccagtgctgcaatgataccgcgagacccacgctcaccggctccagattta |
| | | tcagcaataaaccagccagccggaagggccgagcgcagaagtggtcctgca |
| | | actttatccgcctccatccagtctattaattgttgccgggaagctagagta |
| | | agtagttcgccagttaatagtttgcgcaacgttgttgccattgctacaggc |
| | | atcgtggtgtcacgctcgtcgtttggtatggcttcattcagctccggttcc |
| | | caacgatcaaggcgagttacatgatcccccatgttgtgcaaaaaagcggtt |
| | | agctccttcggtcctccgatcgttgtcagaagtaagttggccgcagtgtta |
| | | tcactcatggttatggcagcactgcataattctcttactgtcatgccatcc |
| | | gtaagatgcttttctgtgactggtgagtactcaaccaagtcattctgagaa |
| | | tagtgtatgcggcgaccgagttgctcttgcccggcgtcaatacgggataat |
| | | accgcgccacatagcagaactttaaaagtgctcatcattggaaaacgttct |
| | | tcggggcgaaaactctcaaggatcttaccgctgttgagatccagttcgatg |
| | | taacccactcgtgcacccaactgatcttcagcatcttttactttcaccagc |
| | | gtttctgggtgagcaaaaacaggaaggcaaaatgccgcaaaaaagggaata |
| | | agggcgacacggaaatgttgaatactcatactcttcctttttcaatattat |
| | | tgaagcatttatcagggttattgtctcatgagcggatacatatttgaatgt |
| | | atttagaaaaataaacaaataggggttccgcgcacatttccccgaaaagtg |
| | | ccacctgagctctaatacgactcactataggacatttgcttctgacacaac |
| | | tgtgttcactagcaacctcaaacagacaccatggaggacgctaaaaatatc |
| | | aagaagggtcccgctcctttctatccactcgaggacggtaccgctggtgag |
| | | caactgcacaaggctatgaaacggtatgctcttgtgccaggcaccatagct |
| | | ttcaccgacgcccacatagaggtggacattacttacgcagagtactttgag |
| | | atgtctgtccggctggcagaggcaatgaaaagatatggcctcaacacaaac |
| | | cataggatagtggtttgcagcgagaattctcttcagttttttatgcccgtc |
| | | ctcggggctcttttcatcggcgtggctgtcgcccccgccaacgacatatac |
| | | aacgagcgagagctgctgaacagcatgggaatcagtcagcctacagtggtt |
| | | tttgtatcaaagaagggcttgcaaaagatccttaacgtacagaaaaagctc |
| | | cctattattcagaagatcattataatggattcaaagaccgactatcaggga |
| | | ttccagtcaatgtatactttcgtcacttcccaccttcctcctgggtttaat |
| | | gaatacgacttcgttcccgaaagtttcgatcgggacaaaactattgcactc |
| | | atcatgaactcctctgggtccactgggctgcctaaaggtgtcgcacttccc |
| | | cacaggactgcatgtgtaaggttctctcacgcacgagatcccatattcggt |
| | | aaccaaatcattcctgataccgctatcctctctgtagtgcctttccaccat |
| | | ggattcggcatgttcaccactctgggttacttgatatgtgggttccgcgtg |
| | | gtccttatgtacagatttgaagaggagctctttttgaggagcttgcaggac |
| | | tacaaaatccaaagtgctctcctcgtccccacattgttctcattcttcgcc |
| | | aaatctactctcatcgacaagtacgatttgagcaacttgcacgaaattgct |
| | | tctgggggggctccactctccaaagaagtcggcgaagccgttgctaaaagg |
| | | ttccacttgccaggcattcgccaggggtatggccttactgagacaacctca |
| | | gctattctcattacccctgagggtgacgataagcctggtgccgtcggtaag |
| | | gtagtaccatttttcgaggcaaaggtggtcgacttggataccggaaaaacc |
| | | ctcggtgtgaatcagagaggcgaattgtgtgtcaggggtccaatgatcatg |
| | | agcggttacgtcaataatcccgaggccactaacgccttgattgataaagat |
| | | ggttggcttcattcaggtgatatagcatactgggatgaggatgaacatttc |
| | | ttcattgtcgataggctcaaaagcctgatcaagtataagggttaccaagtg |
| | | gcccctgccgaactcgaatctatactgctgcaacatccaaacatttttgac |
| | | gctggcgtggccggactgcccgatgatgatgctggtgaactccctgccgcc |
| | | gttgtcgtcctggaacatggtaagaccatgacagaaaaggagattgtagat |
| | | tacgtggcttcacaagtgactacagctaagaaattgcgcgggggtgtcgtt |
| | | ttcgtggacgaggtacccaagggcctcactgggaagctcgacgccagaaaa |
| | | atccgagaaatcctgattaaagcaaagaagggcggaaagattgcagtgtag |
| Nucleic Acid | 40 | gctcgctttcttgctgtccaatttctattaaaggttcctttgttccctaag |
| Template, | | tccaactactaaactgggggatattatgaagggccttgagcatctggattc |
| mRNA | | tgcctaataaaaaacatttattttcattgcaaagccacaccctggagctag |
| encoding VEE | | caaaaaaaaaaaaaaaaaaaaaaaaaaaaaaaaaaaaaaaaaaaaaaaacg |
| structural | | cgtatcgatgatatcgcggccgcatacagcagcaattggcaagctgcttac |
| proteins | | atagaactcgcggcgattggcatgccgccttaaaatttttattttattttt |
| | | cttttcttttccgaatcggattttgtttttaatatttcaaaaaaaaaaaaa |
| | | aaaaaaaaaaaaacgcgtgataacgcaggaaagaacatgtgagcaaaaggc |
| | | cagcaaaaggccaggaaccgtaaaaaggccgcgttgctggcgtttttccat |
| | | aggctccgcccccctgacgagcatcacaaaaatcgacgctcaagtcagagg |
| | | tggcgaaacccgacaggactataaagataccaggcgtttccccctggaagc |
| | | tccctcgtgcgctctcctgttccgaccctgccgcttaccggatacctgtcc |
| | | gcctttctcccttcgggaagcgtggcgctttctcatagctcacgctgtagg |
| | | tatctcagttcggtgtaggtcgttcgctccaagctgggctgtgtgcacgaa |
| | | ccccccgttcagcccgaccgctgcgccttatccggtaactatcgtcttgag |
| | | tccaacccggtaagacacgacttatcgccactggcagcagccactggtaac |
| | | aggattagcagagcgaggtatgtaggcggtgctacagagttcttgaagtgg |
| | | tggcctaactacggctacactagaagaacagtatttggtatctgcgctctg |
| | | ctgaagccagttaccttcggaaaaagagttggtagctcttgatccggcaaa |
| | | caaaccaccgctggtagcggtttttttgtttgcaagcagcagattacgcgc |
| | | agaaaaaaaggatctcaagaagatcctttgatcttttctacggggtctgac |
| | | gctcagtggaacgaaaactcacgttaagggattttggtcatgagattatca |
| | | aaaaggatcttcacctagatccttttaaattaaaaatgaagttttaaatca |
| | | atctaaagtatatatgagtaaacttggtctgacagttaccaatgcttaatc |
| | | agtgaggcacctatctcagcgatctgtctatttcgttcatccatagttgcc |
| | | tgactccccgtcgtgtagataactacgatacgggagggcttaccatctggc |
| | | cccagtgctgcaatgataccgcgagacccacgctcaccggctccagattta |
| | | tcagcaataaaccagccagccggaagggccgagcgcagaagtggtcctgca |
| | | actttatccgcctccatccagtctattaattgttgccgggaagctagagta |
| | | agtagttcgccagttaatagtttgcgcaacgttgttgccattgctacaggc |
| | | atcgtggtgtcacgctcgtcgtttggtatggcttcattcagctccggttcc |
| | | caacgatcaaggcgagttacatgatcccccatgttgtgcaaaaaagcggtt |
| | | agctccttcggtcctccgatcgttgtcagaagtaagttggccgcagtgtta |
| | | tcactcatggttatggcagcactgcataattctcttactgtcatgccatcc |
| | | gtaagatgcttttctgtgactggtgagtactcaaccaagtcattctgagaa |
| | | tagtgtatgcggcgaccgagttgctcttgcccggcgtcaatacgggataat |
| | | accgcgccacatagcagaactttaaaagtgctcatcattggaaaacgttct |
| | | tcggggcgaaaactctcaaggatcttaccgctgttgagatccagttcgatg |
| | | taacccactcgtgcacccaactgatcttcagcatcttttactttcaccagc |
| | | gtttctgggtgagcaaaaacaggaaggcaaaatgccgcaaaaaagggaata |
| | | agggcgacacggaaatgttgaatactcatactcttcctttttcaatattat |
| | | tgaagcatttatcagggttattgtctcatgagcggatacatatttgaatgt |
| | | atttagaaaaataaacaaataggggttccgcgcacatttccccgaaaagtg |
| | | ccacctgagctctaatacgactcactataggacatttgcttctgacacaac |
| | | tgtgttcactagcaacctcaaacagacaccatgttcccgttccagccaatg |
| | | tatccgatgcagccaatgccctatcgcaacccgttcgcggccccgcgcagg |
| | | ccctggttccccagaaccgacccttttctggcgatgcaggtgcaggaatta |
| | | acccgctcgatggctaacctgacgttcaagcaacgccgggacgcgccacct |
| | | gaggggccatccgctaagaaaccgaagaaggaggcctcgcaaaaacagaaa |
| | | gggggaggccaagggaagaagaagaagaaccaagggaagaagaaggctaag |
| | | acagggccgcctaatccgaaggcacagaatggaaacaagaagaagaccaac |
| | | aagaaaccaggcaagagacagcgcatggtcatgaaattggaatctgacaag |
| | | acgttcccaatcatgttggaagggaagataaacggctacgcttgtgtggtc |
| | | ggagggaagttattcaggccgatgcatgtggaaggcaagatcgacaacgac |
| | | gttctggccgcgcttaagacgaagaaagcatccaaatacgatcttgagtat |
| | | gcagatgtgccacagaacatgcgggccgatacattcaaatacacccatgag |
| | | aaaccccaaggctattacagctggcatcatggagcagtccaatatgaaaat |
| | | gggcgtttcacggtgccgaaaggagttggggccaagggagacagcggacga |
| | | cccattctggataaccagggacgggtggtcgctattgtgctgggaggtgtg |
| | | aatgaaggatctaggacagccctttcagtcgtcatgtggaacgagaaggga |
| | | gttaccgtgaagtatactccggagaactgcgagcaatggtcactagtgacc |
| | | accatgtgtctgctcgccaatgtgacgttcccatgtgctcaaccaccaatt |
| | | tgctacgacagaaaaccagcagagactttggccatgctcagcgttaacgtt |
| | | gacaacccgggctacgatgagctgctggaagcagctgttaagtgccccgga |
| | | aggaaaaggagatccaccgaggagctgtttaatgagtataagctaacgcgc |
| | | ccttacatggccagatgcatcagatgtgcagttgggagctgccatagtcca |
| | | atagcaatcgaggcagtaaagagcgacgggcacgacggttatgttagactt |
| | | cagacttcctcgcagtatggcctggattcctccggcaacttaaagggcagg |
| | | accatgcggtatgacatgcacgggaccattaaagagataccactacatcaa |
| | | gtgtcactctatacatctcgcccgtgtcacattgtggatgggcacggttat |
| | | ttcctgcttgccaggtgcccggcaggggactccatcaccatggaatttaag |
| | | aaagattccgtcagacactcctgctcggtgccgtatgaagtgaaatttaat |
| | | cctgtaggcagagaactctatactcatcccccagaacacggagtagagcaa |
| | | gcgtgccaagtctacgcacatgatgcacagaacagaggagcttatgtcgag |
| | | atgcacctcccgggctcagaagtggacagcagtttggtttccttgagcggc |
| | | agttcagtcaccgtgacacctcctgatgggactagcgccctggtggaatgc |
| | | gagtgtggcggcacaaagatctccgagaccatcaacaagacaaaacagttc |
| | | agccagtgcacaaagaaggagcagtgcagagcatatcggctgcagaacgat |
| | | aagtgggtgtataattctgacaaactgcccaaagcagcgggagccacctta |
| | | aaaggaaaactgcatgtcccattcttgctggcagacggcaaatgcaccgtg |
| | | cctctagcaccagaacctatgataaccttcggtttcagatcagtgtcactg |
| | | aaactgcaccctaagaatcccacatatctaatcacccgccaacttgctgat |
| | | gagcctcactacacgcacgagctcatatctgaaccagctgttaggaatttt |
| | | accgtcaccgaaaaagggtgggagtttgtatggggaaaccacccgccgaaa |
| | | aggttttgggcacaggaaacagcacccggaaatccacatgggctaccgcac |
| | | gaggtgataactcattattaccacagataccctatgtccaccatcctgggt |
| | | ttgtcaatttgtgccgccattgcaaccgtttccgttgcagcgtctacctgg |
| | | ctgttttgcagatctagagttgcgtgcctaactccttaccggctaacacct |
| | | aacgctaggataccattttgtctggctgtgctttgctgcgcccgcactgcc |
| | | cgggccgagaccacctgggagtccttggatcacctatggaacaataaccaa |
| | | cagatgttctggattcaattgctgatccctctggccgccttgatcgtagtg |
| | | actcgcctgctcaggtgcgtgtgctgtgtcgtgccttttttagtcatggcc |
| | | ggcgccgcaggcgccggcgcctacgagcacgcgaccacgatgccgagccaa |
| | | gcgggaatctcgtataacactatagtcaacagagcaggctacgcaccactc |
| | | cctatcagcataacaccaacaaagatcaagctgatacctacagtgaacttg |
| | | gagtacgtcacctgccactacaaaacaggaatggattcaccagccatcaaa |
| | | tgctgcggatctcaggaatgcactccaacttacaggcctgatgaacagtgc |
| | | aaagtcttcacaggggtttacccgttcatgtggggtggtgcatattgcttt |
| | | tgcgacactgagaacacccaagtcagcaaggcctacgtaatgaaatctgac |
| | | gactgccttgcggatcatgctgaagcatataaagcgcacacagcctcagtg |
| | | caggcgttcctcaacatcacagtgggagaacactctattgtgactaccgtg |
| | | tatgtgaatggagaaactcctgtgaatttcaatggggtcaaaataactgca |
| | | ggtccgctttccacagcttggacaccctttgatcgcaaaatcgtgcagtat |
| | | gccggggagatctataattatgattttcctgagtatggggcaggacaacca |
| | | ggagcatttggagatatacaatccagaacagtctcaagctctgatctgtat |
| | | gccaataccaacctagtgctgcagagacccaaagcaggagcgatccacgtg |
| | | ccatacactcaggcaccttcgggttttgagcaatggaagaaagataaagct |
| | | ccatcattgaaatttaccgcccctttcggatgcgaaatatatacaaacccc |
| | | attcgcgccgaaaactgtgctgtagggtcaattccattagcctttgacatt |
| | | cccgacgccttgttcaccagggtgtcagaaacaccgacactttcagcggcc |
| | | gaatgcactcttaacgagtgcgtgtattcttccgactttggtgggatcgcc |
| | | acggtcaagtactcggccagcaagtcaggcaagtgcgcagtccatgtgcca |
| | | tcagggactgctaccctaaaagaagcagcagtcgagctaaccgagcaaggg |
| | | tcggcgactatccatttctcgaccgcaaatatccacccggagttcaggctc |
| | | caaatatgcacatcatatgttacgtgcaaaggtgattgtcaccccccgaaa |
| | | gaccatattgtgacacaccctcagtatcacgcccaaacatttacagccgcg |
| | | gtgtcaaaaaccgcgtggacgtggttaacatccctgctgggaggatcagcc |
| | | gtaattattataattggcttggtgctggctactattgtggccatgtacgtg |
| | | ctgaccaaccagaaacataattga |
| First nucleic | 41 | cggcgcatgagagaagcccagaccaattacctacccaaaatggagaaagtt |
| acid construct, | | cacgttgacatcgaggaagacagcccattcctcagagctttgcagcggagc |
| sa-mRNA | | ttcccgcagtttgaggtagaagccaagcaggtcactgataatgaccatgct |
| encoding IL12 | | aatgccagagcgttttcgcatctggcttcaaaactgatcgaaacggaggtg |
| (Sam002- | | gacccatccgacacgatccttgacattggaagtgcgcccgcccgcagaatg |
| IL12) | | tattctaagcacaagtatcattgtatctgtccgatgagatgtgcggaagat |
| | | ccggacagattgtataagtatgcaactaagctgaagaaaaactgtaaggaa |
| | | ataactgataaggaattggacaagaaaatgaaggagctggccgccgtcatg |
| | | agcgaccctgacctggaaactgagactatgtgcctccacgacgacgagtcg |
| | | tgtcgctacgaagggcaagtcgctgtttaccaggatgtatacgcggttgac |
| | | ggaccgacaagtctctatcaccaagccaataagggagttagagtcgcctac |
| | | tggataggctttgacaccaccccttttatgtttaagaacttggctggagca |
| | | tatccatcatactctaccaactgggccgacgaaaccgtgttaacggctcgt |
| | | aacataggcctatgcagctctgacgttatggagcggtcacgtagagggatg |
| | | tccattcttagaaagaagtatttgaaaccatccaacaatgttctattctct |
| | | gttggctcgaccatctaccacgagaagagggacttactgaggagctggcac |
| | | ctgccgtctgtatttcacttacgtggcaagcaaaattacacatgtcggtgt |
| | | gagactatagttagttgcgacgggtacgtcgttaaaagaatagctatcagt |
| | | ccaggcctgtatgggaagccttcaggctatgctgctacgatgcaccgcgag |
| | | ggattcttgtgctgcaaagtgacagacacattgaacggggagagggtctct |
| | | tttcccgtgtgcacgtatgtgccagctacattgtgtgaccaaatgactggc |
| | | atactggcaacagatgtcagtgcggacgacgcgcaaaaactgctggttggg |
| | | ctcaaccagcgtatagtcgtcaacggtcgcacccagagaaacaccaatacc |
| | | atgaaaaattaccttttgcccgtagtggcccaggcatttgctaggtgggca |
| | | aaggaatataaggaagatcaagaagatgaaaggccactaggactacgagat |
| | | agacagttagtcatggggtgttgttgggcttttagaaggcacaagataaca |
| | | tctatttataagcgcccggatacccaaaccatcatcaaagtgaacagcgat |
| | | ttccactcattcgtgctgcccaggataggcagtaacacattggagatcggg |
| | | ctgagaacaagaatcaggaaaatgttagaggagcacaaggagccgtcacct |
| | | ctcattaccgccgaggacgtacaagaagctaagtgcgcagccgatgaggct |
| | | aaggaggtgcgtgaagccgaggagttgcgcgcagctctaccacctttggca |
| | | gctgatgttgaggagcccactctggaagccgatgtcgacttgatgttacaa |
| | | gaggctggggccggctcagtggagacacctcgtggcttgataaaggttacc |
| | | agctacgatggcgaggacaagatcggctcttacgctgtgctttctccgcag |
| | | gctgtactcaagagtgaaaaattatcttgcatccaccctctcgctgaacaa |
| | | gtcatagtgataacacactctggccgaaaagggcgttatgccgtggaacca |
| | | taccatggtaaagtagtggtgccagagggacatgcaatacccgtccaggac |
| | | tttcaagctctgagtgaaagtgccaccattgtgtacaacgaacgtgagttc |
| | | gtaaacaggtacctgcaccatattgccacacatggaggagcgctgaacact |
| | | gatgaagaatattacaaaactgtcaagcccagcgagcacgacggcgaatac |
| | | ctgtacgacatcgacaggaaacagtgcgtcaagaaagaactagtcactggg |
| | | ctagggctcacaggcgagctggtggatcctcccttccatgaattcgcctac |
| | | gagagtctgagaacacgaccagccgctccttaccaagtaccaaccataggg |
| | | gtgtatggcgtgccaggatcaggcaagtctggcatcattaaaagcgcagtc |
| | | accaaaaaagatctagtggtgagcgccaagaaagaaaactgtgcagaaatt |
| | | ataagggacgtcaagaaaatgaaagggctggacgtcaatgccagaactgtg |
| | | gactcagtgctcttgaatggatgcaaacaccccgtagagaccctgtatatt |
| | | gacgaagcttttgcttgtcatgcaggtactctcagagcgctcatagccatt |
| | | ataagacctaaaaaggcagtgctctgcggggatcccaaacagtgcggtttt |
| | | tttaacatgatgtgcctgaaagtgcattttaaccacgagatttgcacacaa |
| | | gtcttccacaaaagcatctctcgccgttgcactaaatctgtgacttcggtc |
| | | gtctcaaccttgttttacgacaaaaaaatgagaacgacgaatccgaaagag |
| | | actaagattgtgattgacactaccggcagtaccaaacctaagcaggacgat |
| | | ctcattctcacttgtttcagagggtgggtgaagcagttgcaaatagattac |
| | | aaaggcaacgaaataatgacggcagctgcctctcaagggctgacccgtaaa |
| | | ggtgtgtatgccgttcggtacaaggtgaatgaaaatcctctgtacgcaccc |
| | | acctcagaacatgtgaacgtcctactgacccgcacggaggaccgcatcgtg |
| | | tggaaaacactagccggcgacccatggataaaaacactgactgccaagtac |
| | | cctgggaatttcactgccacgatagaggagtggcaagcagagcatgatgcc |
| | | atcatgaggcacatcttggagagaccggaccctaccgacgtcttccagaat |
| | | aaggcaaacgtgtgttgggccaaggctttagtgccggtgctgaagaccgct |
| | | ggcatagacatgaccactgaacaatggaacactgtggattattttgaaacg |
| | | gacaaagctcactcagcagagatagtattgaaccaactatgcgtgaggttc |
| | | tttggactcgatctggactccggtctattttctgcacccactgttccgtta |
| | | tccattaggaataatcactgggataactccccgtcgcctaacatgtacggg |
| | | ctgaataaagaagtggtccgtcagctctctcgcaggtacccacaactgcct |
| | | cgggcagttgccactggaagagtctatgacatgaacactggtacactgcgc |
| | | aattatgatccgcgcataaacctagtacctgtaaacagaagactgcctcat |
| | | gctttagtcctccaccataatgaacacccacagagtgacttttcttcattc |
| | | gtcagcaaattgaagggcagaactgtcctggtggtcggggaaaagttgtcc |
| | | gtcccaggcaaaatggttgactggttgtcagaccggcctgaggctaccttc |
| | | agagctcggctggatttaggcatcccaggtgatgtgcccaaatatgacata |
| | | atatttgttaatgtgaggaccccatataaataccatcactatcagcagtgt |
| | | gaagaccatgccattaagcttagcatgttgaccaagaaagcttgtctgcat |
| | | ctgaatcccggcggaacctgtgtcagcataggttatggttacgctgacagg |
| | | gccagcgaaagcatcattggtgctatagcgcggcagttcaagttttcccgg |
| | | gtatgcaaaccgaaatcctcacttgaagagacggaagttctgtttgtattc |
| | | attgggtacgatcgcaaggcccgtacgcacaattcttacaagctttcatca |
| | | accttgaccaacatttatacaggttccagactccacgaagccggatgtgca |
| | | ccctcatatcatgtggtgcgaggggatattgccacggccaccgaaggagtg |
| | | attataaatgctgctaacagcaaaggacaacctggcggaggggtgtgcgga |
| | | gcgctgtataagaaattcccggaaagcttcgatttacagccgatcgaagta |
| | | ggaaaagcgcgactggtcaaaggtgcagctaaacatatcattcatgccgta |
| | | ggaccaaacttcaacaaagtttcggaggttgaaggtgacaaacagttggca |
| | | gaggcttatgagtccatcgctaagattgtcaacgataacaattacaagtca |
| | | gtagcgattccactgttgtccaccggcatcttttccgggaacaaagatcga |
| | | ctaacccaatcattgaaccatttgctgacagctttagacaccactgatgca |
| | | gatgtagccatatactgcagggacaagaaatgggaaatgactctcaaggaa |
| | | gcagtggctaggagagaagcagtggaggagatatgcatatccgacgactct |
| | | tcagtgacagaacctgatgcagagctggtgagggtgcatccgaagagttct |
| | | ttggctggaaggaagggctacagcacaagcgatggcaaaactttctcatat |
| | | ttggaagggaccaagtttcaccaggcggccaaggatatagcagaaattaat |
| | | gccatgtggcccgttgcaacggaggccaatgagcaggtatgcatgtatatc |
| | | ctcggagaaagcatgagcagtattaggtcgaaatgccccgtcgaagagtcg |
| | | gaagcctccacaccacctagcacgctgccttgcttgtgcatccatgccatg |
| | | actccagaaagagtacagcgcctaaaagcctcacgtccagaacaaattact |
| | | gtgtgctcatcctttccattgccgaagtatagaatcactggtgtgcagaag |
| | | atccaatgctcccagcctatattgttctcaccgaaagtgcctgcgtatatt |
| | | catccaaggaagtatctcgtggaaacaccaccggtagacgagactccggag |
| | | ccatcggcagagaaccaatccacagaggggacacctgaacaaccaccactt |
| | | ataaccgaggatgagaccaggactagaacgcctgagccgatcatcatcgaa |
| | | gaggaagaagaggatagcataagtttgctgtcagatggcccgacccaccag |
| | | gtgctgcaagtcgaggcagacattcacgggccgccctctgtatctagctca |
| | | tcctggtccattcctcatgcatccgactttgatgtggacagtttatccata |
| | | cttgacaccctggagggagctagcgtgaccagcggggcaacgtcagccgag |
| | | actaactcttacttcgcaaagagtatggagtttctggcgcgaccggtgcct |
| | | gcgcctcgaacagtattcaggaaccctccacatcccgctccgcgcacaaga |
| | | acaccgtcacttgcacccagcagggcctgctcgagaaccagcctagtttcc |
| | | accccgccaggcgtgaatagggtgatcactagagaggagctcgaggcgctt |
| | | accccgtcacgcactcctagcaggtcggtctcgagaaccagcctggtctcc |
| | | aacccgccaggcgtaaatagggtgattacaagagaggagtttgaggcgttc |
| | | gtagcacaacaacaatgacggtttgatgcgggtgcatacatcttttcctcc |
| | | gacaccggtcaagggcatttacaacaaaaatcagtaaggcaaacggtgcta |
| | | tccgaagtggtgttggagaggaccgaattggagatttcgtatgccccgcgc |
| | | ctcgaccaagaaaaagaagaattactacgcaagaaattacagttaaatccc |
| | | acacctgctaacagaagcagataccagtccaggaaggtggagaacatgaaa |
| | | gccataacagctagacgtattctgcaaggcctagggcattatttgaaggca |
| | | gaaggaaaagtggagtgctaccgaaccctgcatcctgttcctttgtattca |
| | | tctagtgtgaaccgtgccttttcaagccccaaggtcgcagtggaagcctgt |
| | | aacgccatgttgaaagagaactttccgactgtggcttcttactgtattatt |
| | | ccagagtacgatgcctatttggacatggttgacggagcttcatgctgctta |
| | | gacactgccagtttttgccctgcaaagctgcgcagctttccaaagaaacac |
| | | tcctatttggaacccacaatacgatcggcagtgccttcagcgatccagaac |
| | | acgctccagaacgtcctggcagctgccacaaaaagaaattgcaatgtcacg |
| | | caaatgagagaattgcccgtattggattcggcggcctttaatgtggaatgc |
| | | ttcaagaaatatgcgtgtaataatgaatattgggaaacgtttaaagaaaac |
| | | cccatcaggcttactgaagaaaacgtggtaaattacattaccaaattaaaa |
| | | ggaccaaaagctgctgctctttttgcgaagacacataatttgaatatgttg |
| | | caggacataccaatggacaggtttgtaatggacttaaagagagacgtgaaa |
| | | gtgactccaggaacaaaacatactgaagaacggcccaaggtacaggtgatc |
| | | caggctgccgatccgctagcaacagcgtatctgtgcggaatccaccgagag |
| | | ctggttaggagattaaatgcggtcctgcttccgaacattcatacactgttt |
| | | gatatgtcggctgaagactttgacgctattatagccgagcacttccagcct |
| | | ggggattgtgttctggaaactgacatcgcgtcgtttgataaaagtgaggac |
| | | gacgccatggctctgaccgcgttaatgattctggaagacttaggtgtggac |
| | | gcagagctgttgacgctgattgaggcggctttcggcgaaatttcatcaata |
| | | catttgcccactaaaactaaatttaaattcggagccatgatgaaatctgga |
| | | atgttcctcacactgtttgtgaacacagtcattaacattgtaatcgcaagc |
| | | agagtgttgagagaacggctaaccggatcaccatgtgcagcattcattgga |
| | | gatgacaatatcgtgaaaggagtcaaatcggacaaattaatggcagacagg |
| | | tgcgccacctggttgaatatggaagtcaagattatagatgctgtggtgggc |
| | | gagaaagcgccttatttctgtggagggtttattttgtgtgactccgtgacc |
| | | ggcacagcgtgccgtgtggcagaccccctaaaaaggctgtttaagcttggc |
| | | aaacctctggcagcagacgatgaacatgatgatgacaggagaagggcattg |
| | | catgaagagtcaacacgctggaaccgagtgggtattctttcagagctgtgc |
| | | aaggcagtagaatcaaggtatgaaaccgtaggaacttccatcatagttatg |
| | | gccatgactactctagctagcagtgttaaatcattcagctacctgagaggg |
| | | gcccctataactctctacggctaacctgaatggactacgacatagtctagt |
| | | ccgccaagttcgaaggcgcgcctctagagccaccatggactggacctggcg |
| | | aatactgttcttggttgccgccgctacagggactcacgcaatgtgggagct |
| | | tgaaaaagacgtctatgtagtagaagtggactggacacctgatgctcctgg |
| | | cgagacagttaacctcacatgcgatacccctgaggaagatgatatcacctg |
| | | gacttctgaccagagacacggggtgattgggagcggcaaaaccctgacgat |
| | | cactgtgaaggagtttctggacgccggccagtatacctgtcacaagggggg |
| | | ggagaccctgagtcatagccacctgttgctgcacaagaaggagaatggcat |
| | | ctggtctacagagatcctgaagaactttaagaacaagaccttcctgaagtg |
| | | tgaagcaccaaactacagtggtcgctttacctgcagctggctggtccaaag |
| | | aaacatggacctgaaatttaatataaagagtagttcctcgagtcctgattc |
| | | cagggccgtgacgtgcggcatggcaagcctttcagccgaaaaagtcacgct |
| | | ggatcagcgagactatgagaagtacagcgttagctgtcaggaggacgtaac |
| | | ttgcccgactgccgaggagactctgcccatagagctcgctctggaggccag |
| | | gcagcagaacaaatatgagaattacagcactagtttctttattagagacat |
| | | catcaaacccgacccacccaagaatctgcagatgaagccgctgaagaatag |
| | | tcaggtcgaggtttcctgggaatatccagattcatggtccactccgcattc |
| | | ttatttttccttaaaattctttgttaggattcagcggaaaaaagaaaagat |
| | | gaaagagacggaggaagggtgcaaccagaagggggccttcctggtggaaaa |
| | | gacaagcactgaggtccaatgtaagggtgggaacgtttgcgtgcaggctca |
| | | ggatcgctactacaacagcagttgctctaagtgggcctgcgtaccttgtcg |
| | | cgtcaggagtggaggggggtcagggggtggctcaggcggcggcagtggggg |
| | | cagcagggtgatcccagtgtctgggccggcccgttgcttgtctcaatccag |
| | | aaacctcctcaagaccactgacgatatggtaaagactgcccgagagaagct |
| | | aaaacactactcttgtacagctgaagatatagaccatgaggatataacacg |
| | | ggaccagacctctactctgaaaacctgtctgcctcttgagctgcacaagaa |
| | | cgagtcctgtctggctacccgcgaaacctcaagcacaaccagaggtagttg |
| | | cctgcccccacaaaagacatcgcttatgatgaccttgtgtctgggatctat |
| | | ttatgaggacctgaagatgtaccaaactgagttccaggcaataaatgctgc |
| | | tctccagaatcacaatcatcaacaaatcatccttgataaggggatgctggt |
| | | cgcaatcgacgagctcatgcaatcgctgaaccacaatggggaaaccctcag |
| | | gcagaaaccaccggtgggagaggccgacccctaccgtgttaaaatgaagtt |
| | | gtgtattcttttgcatgcattctctacaagagtcgttaccatcaatcgcgt |
| | | catggggtacctgtcatcagcctagcatcgatgatatcgcggccgcataca |
| | | gcagcaattggcaagctgcttacatagaactcgcggcgattggcatgccgc |
| | | cttaaaatttttattttatttttcttttcttttccgaatcggattttgttt |
| | | ttaatatttcaaaaaaaaaaaaaaaaaaaaaaaaaacgcggaagagc |
| First nucleic | 42 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding GFP | | taatgccagagcgttttcgcatctggcttcaaaactgatcgaaacggaggt |
| (Sam002-GFP) | | ggacccatccgacacgatccttgacattggaagtgcgcccgcccgcagaat |
| | | gtattctaagcacaagtatcattgtatctgtccgatgagatgtgcggaaga |
| | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatggactacgacatagtctag |
| | | tccgccaagttcgaaggcgcgcctctagagccaccatggtgagcaagggcg |
| | | aggagctgttcaccggggtggtgcccatcctggtcgagctggacggcgacg |
| | | taaacggccacaagttcagcgtgtccggcgagggcgagggcgatgccacct |
| | | acggcaagctgaccctgaagttcatctgcaccaccggcaagctgcccgtgc |
| | | cctggcccaccctcgtgaccaccctgacctacggcgtgcagtgcttcagcc |
| | | gctaccccgaccacatgaagcagcacgacttcttcaagtccgccatgcccg |
| | | aaggctacgtccaggagcgcaccatcttcttcaaggacgacggcaactaca |
| | | agacccgcgccgaggtgaagttcgagggcgacaccctggtgaaccgcatcg |
| | | agctgaagggcatcgacttcaaggaggacggcaacatcctggggcacaagc |
| | | tggagtacaactacaacagccacaacgtctatatcatggccgacaagcaga |
| | | agaacggcatcaaggtgaacttcaagatccgccacaacatcgaggacggca |
| | | gcgtgcagctcgccgaccactaccagcagaacacccccatcggcgacggcc |
| | | ccgtgctgctgcccgacaaccactacctgagcacccagtccgccctgagca |
| | | aagaccccaacgagaagcgcgatcacatggtcctgctggagttcgtgaccg |
| | | ccgccgggatcactctcggcatggacgagctgtacaagtagcatcgatgat |
| | | atcgcggccgcatacagcagcaattggcaagctgcttacatagaactcgcg |
| | | gcgattggcatgccgccttaaaatttttattttatttttcttttcttttcc |
| | | gaatcggattttgtttttaatatttcaaaaaaaaaaaaaaaaaaaaaaaaa |
| | | a |
| Second nucleic | 43 | acatttgcttctgacacaactgtgttcactagcaacctcaaacagacacca |
| acid construct, | | tggaggacgctaaaaatatcaagaagggtcccgctcctttctatccactcg |
| mRNA | | aggacggtaccgctggtgagcaactgcacaaggctatgaaacggtatgctc |
| encoding | | ttgtgccaggcaccatagctttcaccgacgcccacatagaggtggacatta |
| luciferase | | cttacgcagagtactttgagatgtctgtccggctggcagaggcaatgaaaa |
| | | gatatggcctcaacacaaaccataggatagtggtttgcagcgagaattctc |
| | | ttcagttttttatgcccgtcctcggggctcttttcatcggcgtggctgtcg |
| | | cccccgccaacgacatatacaacgagcgagagctgctgaacagcatgggaa |
| | | tcagtcagcctacagtggtttttgtatcaaagaagggcttgcaaaagatcc |
| | | ttaacgtacagaaaaagctccctattattcagaagatcattataatggatt |
| | | caaagaccgactatcagggattccagtcaatgtatactttcgtcacttccc |
| | | accttcctcctgggtttaatgaatacgacttcgttcccgaaagtttcgatc |
| | | gggacaaaactattgcactcatcatgaactcctctgggtccactgggctgc |
| | | ctaaaggtgtcgcacttccccacaggactgcatgtgtaaggttctctcacg |
| | | cacgagatcccatattcggtaaccaaatcattcctgataccgctatcctct |
| | | ctgtagtgcctttccaccatggattcggcatgttcaccactctgggttact |
| | | tgatatgtgggttccgcgtggtccttatgtacagatttgaagaggagctct |
| | | ttttgaggagcttgcaggactacaaaatccaaagtgctctcctcgtcccca |
| | | cattgttctcattcttcgccaaatctactctcatcgacaagtacgatttga |
| | | gcaacttgcacgaaattgcttctgggggggctccactctccaaagaagtcg |
| | | gcgaagccgttgctaaaaggttccacttgccaggcattcgccaggggtatg |
| | | gccttactgagacaacctcagctattctcattacccctgagggtgacgata |
| | | agcctggtgccgtcggtaaggtagtaccatttttcgaggcaaaggtggtcg |
| | | acttggataccggaaaaaccctcggtgtgaatcagagaggcgaattgtgtg |
| | | tcaggggtccaatgatcatgagcggttacgtcaataatcccgaggccacta |
| | | acgccttgattgataaagatggttggcttcattcaggtgatatagcatact |
| | | gggatgaggatgaacatttcttcattgtcgataggctcaaaagcctgatca |
| | | agtataagggttaccaagtggcccctgccgaactcgaatctatactgctgc |
| | | aacatccaaacatttttgacgctggcgtggccggactgcccgatgatgatg |
| | | ctggtgaactccctgccgccgttgtcgtcctggaacatggtaagaccatga |
| | | cagaaaaggagattgtagattacgtggcttcacaagtgactacagctaaga |
| | | aattgcgcgggggtgtcgttttcgtggacgaggtacccaagggcctcactg |
| | | ggaagctcgacgccagaaaaatccgagaaatcctgattaaagcaaagaagg |
| | | gcggaaagattgcagtgtaggctcgctttcttgctgtccaatttctattaa |
| | | aggttcctttgttccctaagtccaactactaaactgggggatattatgaag |
| | | ggccttgagcatctggattctgcctaataaaaaacatttattttcattgca |
| | | aagccacaccctggagctagcaaaaaaaaaaaaaaaaaaaaaaaaaaaaaa |
| | | aaaaaaaaaaaaaaaaaa |
| GOI, sa- | 44 | atggactggacctggcgaatactgttcttggttgccgccgctacagggact |
| mRNA | | cacgcaatatgggagctgaagaaggacgtgtatgtcgtggagctggactgg |
| encoding | | tacccagatgctcctggcgaaatggtggttttaacatgtgatacccccgag |
| human IL12 | | gaggacggcatcacatggactctggaccagagttctgaggtgctggggtcc |
| | | ggcaagactctgacaatccaggttaaggagttcggcgacgcaggacagtac |
| | | acttgtcacaagggaggtgaggtgctttctcacagcctgttgctgctccat |
| | | aagaaggaagacggtatttggtcaaccgacatcctcaaggaccagaaggag |
| | | cccaaaaacaagacctttctgagatgtgaggccaagaattacagcggtaga |
| | | ttcacttgttggtggctcaccaccatatccacagacttgaccttcagtgtc |
| | | aaaagttcacgagggagctcagatcctcaaggcgttacctgtggcgcagcg |
| | | acgctgtccgcagaaagagtcaggggagacaacaaggaatacgagtactct |
| | | gtcgagtgccaggaggattccgcctgtccggccgcagaggagtctttacct |
| | | attgaggtgatggtcgatgccgtgcacaagcttaagtacgagaattacaca |
| | | tcaagttttttcatccgcgacatcattaaacctgatccaccaaagaacctg |
| | | cagctcaagcctctgaagaatagcaggcaggtcgaggtaagctgggagtat |
| | | cctgatacctggtccaccccccacagttatttcagcctcaccttctgcgtc |
| | | caagtccagggaaagagcaagagagagaagaaggatagggtgttcacagat |
| | | aagacttcagctactgtgatctgcagaaagaatgcgtctatctctgtgcga |
| | | gcacaagacaggtactacagttctagctggagcgagtgggcatcagtcccc |
| | | tgcagtggtggcggaagcggagggggcagcggaggtgggagcggagggagc |
| | | aggaacctcccagttgctacacctgacccgggaatgtttccatgcctccac |
| | | cattcccagaatctcctccgagccgtgtccaatatgctgcaaaaggctcgg |
| | | cagaccttggagttttacccttgcacctcagaagaaatcgatcatgaggat |
| | | atcacaaaggataagacgagcactgttgaggcatgcctgcccctggagcta |
| | | actaagaatgagtcttgcctgaacagcagggagacttccttcattaccaac |
| | | ggtagctgtcttgccagcaggaagacatcttttatgatggccctgtgtcta |
| | | tctagcatatatgaagacctgaagatgtaccaggtggaattcaaaaccatg |
| | | aatgctaagcttctcatggatcccaagaggcaaatcttcctggaccagaat |
| | | atgcttgctgtcatagatgaactgatgcaggcgttgaattttaacagcgag |
| | | acggtgcctcaaaaaagctcactggaagaacctgatttttataaaacgaag |
| | | atcaagctgtgtattttactacacgcctttagaatccgcgctgttaccatc |
| | | gacagagtaatgtcctacctaaatgcttcatag |
| GOI, human | SEQ | atgtaccgcatgcagttgctgtcttgtattgcgcttagcctcgcgttggtc |
| IL2 | ID | acaaacagtgccccgacctccagttctaccaagaagacgcaactccagctt |
| | NO. | gagcatcttctcctggacttgcagatgatattgaatggtatcaacaattac |
| | 45 | aagaatcccaaactcaccaggatgcttacttttaagttttatatgcctaag |
| | | aaagcgacggaactgaaacaccttcaatgcttggaggaagaactgaaaccg |
| | | cttgaggaggtactgaatttggcacagtcaaaaaacttccacctgaggcca |
| | | agggacttgatcagtaacataaacgtaatcgttttggagcttaaggggagc |
| | | gaaaccacctttatgtgcgagtacgcagacgagacggccacgattgtagaa |
| | | tttttgaatcgctggattactttttgccaaagtatcatcagtacactcacc |
| | | tag |
| GOI, mouse | SEQ | atgtattcaatgcagcttgccagttgtgtcacactcactcttgtcctgctg |
| IL2 | ID | gttaattccgccccaacttcaagccccaccagctcccccacctcaagttca |
| | NO. | accgctgaagcacagcaacagcaacaacaacagcaacacttggaacaactc |
| | 46 | ctcatggatctccaggaactgctttctcgaatggagaactaccggaatctg |
| | | aaacttcccagaatgctgaccttcaagttctacctccctaagcaggctact |
| | | gaacttaaagatttgcaatgtcttgaagatgaactgggcccacttcgacac |
| | | gtattggatctgacccagtcaaaatcctttcaattggaggacgcagagaac |
| | | ttcataagcaacatcagggtcaccgtggtgaagctcaaggggtctgacaat |
| | | acatttgaatgtcaatttgatgatgagtcagccaccgtcgtcgattttttg |
| | | cggagatggatcgcattctgccagagcataatttctaccagccctcagtag |
| GOI, human | SEQ | atgtatcgaatgcaattgctgtcctgcattgcgctctctctggcgcttgtc |
| IL15 | ID | accaattcaaactgggttaacgtaatatctgatttgaaaaaaatcgaagac |
| | NO. | ctcatacagagtatgcacatagatgctacgctgtacacagaaagcgacgta |
| | 47 | cacccgagctgtaaggtcactgccatgaagtgctttcttctcgaactccaa |
| | | gttatcagtctcgaatccggggatgcgtcaatccatgacactgttgagaat |
| | | ctcattattctcgcgaacaactcactcagctcaaatgggaacgtcactgaa |
| | | tccggctgcaaggaatgcgaggaattggaagagaagaatattaaagagttc |
| | | ctgcagagtttcgttcatattgttcaaatgtttatcaacacaagttag |
| GOI, mouse | SEQ | atgtattcaatgcagcttgccagttgtgtcacactcactcttgtcctgctg |
| IL15 | ID | gttaattccaattggatcgatgtccggtatgatttggagaagatcgaatct |
| | NO. | ctcatccaaagcatacacattgacaccactttgtatactgatagcgatttt |
| | 48 | cacccatcttgtaaggtgaccgccatgaattgctttctgttggaacttcaa |
| | | gttatacttcacgagtattcaaacatgacactgaatgaaactgtccgaaac |
| | | gtactctaccttgctaactccaccctttcatctaataagaacgtagctgag |
| | | agcggatgtaaggagtgtgaggagctcgaagagaagacatttactgaattt |
| | | cttcagagttttatccgcatagtgcaaatgtttattaacacctcctag |
| GOI, human | SEQ | atgtatcgaatgcaattgctgtcctgcattgcgctctctctggcgcttgtc |
| IL15/IL15Rα | ID | accaattcaaactgggttaacgtaatatctgatttgaaaaaaatcgaagac |
| | NO. | ctcatacagagtatgcacatagatgctacgctgtacacagaaagcgacgta |
| | 49 | cacccgagctgtaaggtcactgccatgaagtgctttcttctcgaactccaa |
| | | gttatcagtctcgaatccggggatgcgtcaatccatgacactgttgagaat |
| | | ctcattattctcgcgaacaactcactcagctcaaatgggaacgtcactgaa |
| | | tccggctgcaaggaatgcgaggaattggaagagaagaatattaaagagttc |
| | | ctgcagagtttcgttcatattgttcaaatgtttatcaacacaagtggaggg |
| | | gggtcagggggtggctcaggcggcggcagtgggggcagcataacttgtcca |
| | | cctccgatgagtgtggaacatgctgacatttgggtaaagagttactcactc |
| | | tattcacgagagcgatacatctgtaatagtgggtttaaaagaaaagccggt |
| | | acatcctctcttactgaatgcgtactcaataaggcgaccaacgtcgcgcac |
| | | tggaccactccttccttgaaatgcatccgcgacccggctctcgtttag |
| GOI, human | SEQ | atgtaccgcatgcaacttttgtcttgcattgcacttagcttggcactcgtc |
| IL15Rα/IL15 | ID | actaattccattacatgccctccgcctatgtctgtggagcacgcagacatc |
| | NO. | tgggtaaagtcttactccctctactcaagagaacgatatatctgtaattca |
| | 50 | gggtttaagcgaaaagcaggaacctcttcactgacagaatgcgtgcttaat |
| | | aaggcgacgaacgttgctcattggactacacctagtttgaagtgtattcgg |
| | | gatccggcactcgtaggaggggggtcagggggtggctcaggcggcggcagt |
| | | gggggcagcaattgggtaaacgtaattagcgatctgaagaagatcgaggac |
| | | ttgatacaaagtatgcatatcgatgctactctttataccgagtctgatgta |
| | | cacccctcttgtaaagtaacagcgatgaagtgctttttgttggagttgcaa |
| | | gtgataagcctggaaagtggggatgcgtccatccacgataccgtcgagaac |
| | | ttgataatactggccaacaacagtctctctagtaacggtaacgtaaccgag |
| | | agcgggtgtaaagaatgcgaagaattggaagagaaaaatatcaaagaattc |
| | | cttcagtcatttgtgcatatagtgcagatgtttatcaacacatcctag |
| GOI, mouse | SEQ | atgtattcaatgcagcttgccagttgtgtcacactcactcttgtcctgctg |
| IL15/IL15Rα | ID | gttaattccaattggatcgatgtccggtatgatttggagaagatcgaatct |
| | NO. | ctcatccaaagcatacacattgacaccactttgtatactgatagcgatttt |
| | 51 | cacccatcttgtaaggtgaccgccatgaattgctttctgttggaacttcaa |
| | | gttatacttcacgagtattcaaacatgacactgaatgaaactgtccgaaac |
| | | gtactctaccttgctaactccaccctttcatctaataagaacgtagctgag |
| | | agcggatgtaaggagtgtgaggagctcgaagagaagacatttactgaattt |
| | | cttcagagttttatccgcatagtgcaaatgtttattaacacctccggaggg |
| | | gggtcagggggtggctcaggcggcggcagtgggggcagcggaactacatgt |
| | | ccacctcccgtcagtatcgaacatgcagatatcagagttaaaaattattca |
| | | gtgaacagcagggagcgctacgtgtgtaatagtggttttaaaaggaaagcc |
| | | ggcacttctacattgattgaatgcgttatcaataagaatactaacgtagct |
| | | cactggaccacaccctctctgaagtgtatccgagacccttcacttgcctag |
| GOI, mouse | SEQ | atgtattcaatgcagcttgccagttgtgtcacactcactcttgtcctgctg |
| IL15Rα/IL15 | ID | gttaattccggaactacttgcccaccacccgtcagtatcgagcacgctgat |
| | NO. | ataagagttaaaaactattctgtaaattctagggaaagatatgtatgtaac |
| | 52 | agcggtttcaaaaggaaggcagggacatcaacccttatcgagtgcgtcatt |
| | | aacaaaaatacaaacgtcgcccattggaccactccatcactcaaatgtata |
| | | cgagaccctagcttggcaggaggggggtcagggggtggctcaggcggcggc |
| | | agtgggggcagcaattggatagatgtccggtacgacctcgagaagatcgag |
| | | tcacttattcaatctatacatattgacactaccctgtatactgacagcgac |
| | | tttcatccaagttgtaaagtcactgctatgaattgctttttgctcgagttg |
| | | caggtcatcctgcatgagtattctaatatgactttgaatgaaaccgtcaga |
| | | aatgtcttgtatcttgctaacagtacccttagctccaacaagaatgttgcc |
| | | gaatcaggttgtaaagaatgcgaggagttggaagagaaaacctttaccgaa |
| | | tttcttcaatcattcatcagaattgtacaaatgttcatcaatacttcatag |
| GOI, mouse | SEQ | atgacctcccggcttgtgagggtactggctgctgctatgctggtggctgct |
| IL18-WT | ID | gctgtgagtgtggcaaactttgggagactccactgtaccactgccgttata |
| | NO. | cggaatataaacgatcaggtcctctttgtagacaagagacagccagttttc |
| | 53 | gaggacatgactgatatagatcagagtgcctctgagcctcaaactcggctc |
| | | ataatatacatgtataaggatagcgaggttcggggacttgccgtgactttg |
| | | tccgtgaaagactcaaaaatgagcaccctgtcatgcaaaaataagataatt |
| | | tctttcgaggaaatggaccctcccgagaacattgatgatatccagtccgac |
| | | ttgatatttttccagaaacgggttcccggtcataacaaaatggagtttgag |
| | | tcttcattgtacgagggtcattttttggcatgccagaaggaggatgatgcc |
| | | ttcaaactgattttgaagaaaaaagacgagaacggagacaagtctgtcatg |
| | | tttacacttaccaaccttcatcaatcctag |
| GOI, mouse | SEQ | atgacctcccggcttgtgagggtactggctgctgctatgctggtggctgct |
| IL18-mutant | ID | gctgtgagtgtggcacacttcggacgactccattgtactacagccgttata |
| | NO. | cgaaatatcaatgatcaggtgttgtttgtagacaaaaggcagcccgtgttt |
| | 54 | gaagacatgactgatattgatcagagtgcctctgagccccagactagactg |
| | | attatatatgcttatggcgactctagagctagagggaaagcagtaactctg |
| | | tcagtgaaagacagcaagatgtctaccctgtcttgcaagaataaaataatc |
| | | tcatttgaggagatggacccccctgaaaacatagatgacatccaaagtgac |
| | | ctgatattctttcagaagcgggtacccggtcataacaaaatggagttcgag |
| | | tcaagcctctatgagggtcactttctcgcctgccaaaaggaagatgacgcc |
| | | ttcaaattgatcttgaaaaaaaaagacgagaatggtgataagtctgtcatg |
| | | ttcacactcactaatttgcatcagtcatag |
| GOI, mouse | SEQ | atgaatccatccgcagctgtaattttctgccttatcctcctggggctgagt |
| mCXCL10-9- | ID | ggaacccaaggaatccccctcgctcgaacagtacgctgcaactgtatccac |
| 11-4 f | NO. | atagacgatggccccgttcgaatgagggctatcggaaagctcgaaataata |
| | 55 | cccgcctctctctcatgcccccgcgtagaaataatagcaactatgaagaag |
| | | aacgacgagcaacgatgcttgaacccagaaagcaagactatcaagaacttg |
| | | atgaaagcttttagccaaaagcggagtaaacgagcccccgggggaggcggc |
| | | tccggcggaggagggagtggtggcggtggatccggaggcggtgggagtacc |
| | | cttgtgattaggaatgcacgatgcagctgtataagcacctcccggggcact |
| | | attcactataagagccttaaagatctcaagcagtttgcaccatctccaaat |
| | | tgtaacaaaactgagattattgctacactgaagaacggggaccaaacctgc |
| | | ctggacccagatagtgccaatgttaaaaagcttatgaaggaatgggagaaa |
| | | aaaatcagtcaaaagaaaaagcaaaaacgcggtaaaaaacaccagaaaaac |
| | | atgaagaatcggaaaccaaaaaccccacagtctaggagaaggagtcgaaaa |
| | | acaacagggggaggcggaagtggcggcgggggcagcggtggcgggggatca |
| | | ggcggcgggggcagtttccttatgttcaaacaagggcggtgcctgtgtata |
| | | gggccaggtatgaaagcagtgaagatggctgaaatcgaaaaagcaagtgtg |
| | | atctaccctagtaatggctgtgataaagttgaagttattgtgactatgaaa |
| | | gctcacaagagacagcggtgtttggatcctagatccaaacaagcccgactc |
| | | atcatgcaagccatagaaaagaagaacttcctgcgaaggcagaatatgggt |
| | | ggaggtggaagcggtggcgggggtagcggcgggggaggtagtggtgggggc |
| | | ggtagcgtaactagtgctggaccagaggagtccgatggggatctctcttgt |
| | | gtctgcgttaagaccatatcatccgggattcaccttaagcacattaccagc |
| | | ctcgaagtgatcaaagctggtaggcattgtgccgtgccccagctcatagcc |
| | | acattgaagaatgggaggaaaatttgccttgatagacaggcaccattgtat |
| | | aagaaagtcattaaaaaaattcttgaatcatag |
| GOI, structural | 56 | atgaattacatccctacgcaaacgttttacgggcgcagatggcgacctcga |
| proteins from | | cccgccgctcgcccctggcctttgcaagctacgcccgtagcccccgtggtc |
| semliki forest | | ccagactttcaggcacagcaaatgcagcagcaactcatcagcgccgtaaat |
| virus SVF4 | | gcgctgacaatgagacagaacgcaattgctcctgctaggcctcccaaacca |
| | | aagaagaagaagacaaccaaaccaaagccgaaaacgcagcccaagaagatc |
| | | aacggaaaaacgcagcagcaaaagaagaaagacaagcaagccgacaagaag |
| | | aagaagaaacccggaaaaagagaaagaatgtgcatgaagattgaaaatgac |
| | | tgtatcttcgaagtcaaacacgaaggaaaggtcactgggtacgcctgcctg |
| | | gtgggcgacaaagtcatgaaacctgcccacgtgaaaggagtcatcgacaac |
| | | gcggacctggcaaagctagctttcaagaaatcgagcaagtatgaccttgag |
| | | tgtgcccagataccagttcacatgaggtcggatgcctcaaagtacacgcat |
| | | gagaagcccgagggacactataactggcaccacggggctgttcagtacagc |
| | | ggaggtaggttcactataccgacaggagcgggcaaaccgggagacagtggc |
| | | cggcccatctttgacaacaaggggagggtagtcgctatcgtcctgggcggg |
| | | gccaacgagggctcacgcacagcactgtcggtggtcacctggaacaaagat |
| | | atggtgactagagtgacccccgaggggtccgaagagtggtccgccccgctg |
| | | attactgccatgtgtgtccttgccaatgctaccttcccgtgcttccagccc |
| | | ccgtgtgtaccttgctgctatgaaaacaacgcagaggccacactacggatg |
| | | ctcgaggataacgtggataggccagggtactacgacctccttcaggcagcc |
| | | ttgacgtgccgaaacggaacaagacaccggcgcagcgtgtcgcaacacttc |
| | | aacgtgtataaggctacacgcccttacatcgcgtactgcgccgactgcgga |
| | | gcagggcactcgtgtcatagccccgtagcaattgaagcggtcaggtccgaa |
| | | gctaccgacgggatgctgaagattcagttctcggcacaaattggcatagat |
| | | aagagtgacaatcatgactacacgaagataaggtacgcagacgggcacgcc |
| | | attgagaatgccgtccggtcatctttgaaggtagccacctccggagactgt |
| | | ttcgtccatggcacaatgggacatttcatactggcaaagtgcccaccgggt |
| | | gaattcctgcaggtctcgatccaggacaccagaaacgcggtccgtgcctgc |
| | | agaatacaatatcatcatgaccctcaaccggtgggtagagaaaaatttaca |
| | | attagaccacactatggaaaagagatcccttgcaccacttatcaacagacc |
| | | acagcgaagaccgtggaggaaatcgacatgcatatgccgccagatacgccg |
| | | gacaggacgttgctatcacagcaatctggcaatgtaaagatcacagtcgga |
| | | ggaaagaaggtgaaatacaactgcacctgtggaaccggaaacgttggcact |
| | | actaattcggacatgacgatcaacacgtgtctaatagagcagtgccacgtc |
| | | tcagtgacggaccataagaaatggcagttcaactcacctttcgtcccgaga |
| | | gccgacgaaccggctagaaaaggcaaagtccatatcccattcccgttggac |
| | | aacatcacatgcagagttccaatggcgcgcgaaccaaccgtcatccacggc |
| | | aaaagagaagtgacactgcaccttcacccagatcatcccacgctcttttcc |
| | | taccgcacactgggtgaggacccgcagtatcacgaggaatgggtgacagcg |
| | | gcggtggaacggaccatacccgtaccagtggacgggatggagtaccactgg |
| | | ggaaacaacgacccagtgaggctttggtctcaactcaccactgaagggaaa |
| | | ccgcacggctggccgcatcagatcgtacagtactactatgggctttacccg |
| | | gccgctacagtatccgcggtcgtcgggatgagcttactggcgttgatatcg |
| | | atcttcgcgtcgtgctacatgctggttgcggcccgcagtaagtgcttgacc |
| | | ccttatgctttaacaccaggagctgcagttccgtggacgctggggatactc |
| | | tgctgcgccccgcgggcgcacgcagctagtgtggcagagactatggcctac |
| | | ttgtgggaccaaaaccaagcgttgttctggttggagtttgcggcccctgtt |
| | | gcctgcatcctcatcatcacgtattgcctcagaaacgtgctgtgttgctgt |
| | | aagagcctttcttttttagtgctactgagcctcggggcaaccgccagagct |
| | | tacgaacattcgacagtaatgccgaacgtggtggggttcccgtataaggct |
| | | cacattgaaaggccaggatatagccccctcactttgcagatgcaggttgtt |
| | | gaaaccagcctcgaaccaacccttaatttggaatacataacctgtgagtac |
| | | aagacggtcgtcccgtcgccgtacgtgaagtgctgcggcgcctcagagtgc |
| | | tccactaaagagaagcctgactaccaatgcaaggtttacacaggcgtgtac |
| | | ccgttcatgtggggaggggcatattgcttctgcgactcagaaaacacgcaa |
| | | ctcagcgaggcgtacgtcgatcgatcggacgtatgcaggcatgatcacgca |
| | | tctgcttacaaagcccatacagcatcgctgaaggccaaagtgagggttatg |
| | | tacggcaacgtaaaccagactgtggatgtttacgtgaacggagaccatgcc |
| | | gtcacgatagggggtactcagttcatattcgggccgctgtcatcggcctgg |
| | | accccgttcgacaacaagatagtcgtgtacaaagacgaagtgttcaatcag |
| | | gacttcccgccgtacggatctgggcaaccagggcgcttcggcgacatccaa |
| | | agcagaacagtggagagtaacgacctgtacgcgaacacggcactgaagctg |
| | | gcacgcccttcacccggcatggtccatgtaccgtacacacagacaccttca |
| | | gggttcaaatattggctaaaggaaaaagggacagccctaaatacgaaggct |
| | | ccttttggctgccaaatcaaaacgaaccctgtcagggccatgaactgcgcc |
| | | gtgggaaacatccctgtctccatgaatttgcctgacagcgcctttacccgc |
| | | attgtcgaggcgccgaccatcattgacctgacttgcacagtggctacctgt |
| | | acgcactcctcggatttcggcggcgtcttgacactgacgtacaagaccgac |
| | | aagaacggggactgctctgtacactcgcactctaacgtagctactctacag |
| | | gaggccacagcaaaagtgaagacagcaggtaaggtgaccttacacttctcc |
| | | acggcaagcgcatcaccttcttttgtggtgtcgctatgcagtgctagggcc |
| | | acctgttcagcgtcgtgtgagcccccgaaagaccacatagtcccatatgcg |
| | | gctagccacagtaacgtagtgtttccagacatgtcgggcaccgcactatca |
| | | tgggtgcagaaaatctcgggtggtctgggggccttcgcaatcggcgctatc |
| | | ctggtgctggttgtggtcacttgcattgggctccgcagataa |
| GOI, structural | 57 | atgaatagaggaatatttaacatgctgagccgccgccccttccccgccccc |
| proteins from a | | acagctatgtggaggcctaggcgaaggaggcaggcggcaccactgcccgcc |
| sindbis virus | | cgtaatgggttggcatcccaaattcaacagctaaccacagctgtcagtgcc |
| | | ctagtcattggtcaagccacccgacctcaacaaccaaggccacgcccgcta |
| | | ccacgccctaagaagcagtccccgaagcaaccaccgaatgctaaaaagaca |
| | | aaaccgcaggagaacaaggagaaacaacccgcaaaaccgaagccgggtaag |
| | | agacagcgtatggcactaaaattgatgctgacaggactctttgacgtgaac |
| | | aacgaagccggtgacgtaataggacacgccctcgctatggaaggcaaagtg |
| | | atgaagccacttcacgtgaaaggggtcatagaccacccggttttggcaaag |
| | | ctgaaatttaccaagtcatcagcgtacgacatggagttcgcccagcttcca |
| | | gccagtatgaaaagcgaagcatttgcatacaccagcgagcaccctgaaggc |
| | | ttctacaactggcatcatggtgccgtgcagtacagcggcggtcgctttaca |
| | | gtcccgcgcggagttgggggaaaaggcgacagtggaagaccgatcatggat |
| | | aatacaggtaaggtggtcgcgatagtgctgggcggcgctgacgaaggcgcc |
| | | cgcactgctctctccgtcgtcacttggaacagcaaaggaaagaccatcaaa |
| | | acgactccggaagggactgaggaatggtctgcagcaccattagtcacggct |
| | | atgtgcttctttgagaatgtatcgttcccgtgcagtcgtccgcctacgtgc |
| | | tattcccgaaaaccatctcgtgcccttgacatcctggaggagaacgtgaac |
| | | aacgacgcctacgataccttattagcggcagtgttgaagtgcaggacgtcc |
| | | ggtcggaacaaacgcagcatcaccgacgatttcaccctgaccagcccgtac |
| | | ctgggcacttgctcgtattgccatcatactgaaccctgcttcagcccaatt |
| | | aagatcgagcaagtttgggacgaggcggacgacggatcaatacgaatacag |
| | | acatccgcccagttcgggtacgaccaaagtggggcagcagacgtcaacaag |
| | | tacaggtacatgtcaattgaccaggaccatgccgtgaaagaaggctctatg |
| | | gatcatatcaagatcagcacatccgggccctgtaggcggcttcaatcacaa |
| | | aggtacttcttgcttgcgaaatgcccaccgggagatagcgttacggttagc |
| | | ataaccggcggcactatggccacctcctgtacattagcacgtaaaataagg |
| | | caaaaattcgtaggacgtgagaagtatgaccttcccccagcacacggaaag |
| | | aacatcccctgcaccgtgtatgatcgtcttaaggagacgtcagccggatac |
| | | atcaccatgcacaggcctggacctcatgcctatacctcatacctagaggct |
| | | acctcaggaaaaatttacgccaaaccaccatcaggaaagaacatcacgtac |
| | | gagtgtaagtgtggcgattataaaactgcgaccgtttccgtcagaacagag |
| | | atcgcaggatgtactgccataaaacagtgcgtggcgtacaagagcgaccag |
| | | accaagtgggtgttcaactcgcccgatctaatcaggcacgctgaccacaca |
| | | gcccaaggtaaattccactttcctttcaagcctaccttcagtacatgcctg |
| | | gtaccactggcacacgagccgactgtaattcatgggttcaaacacataagc |
| | | ctccatttagacacagaccacccaaccttgctcaccattagaagactgggc |
| | | gagaaacccgaaccgacgtcggagtggatctcaggaaaaacagttaggaat |
| | | tttactgtcgacagagacgggctagagtatatatggggcaatcacgaaccg |
| | | gtgagagtatatgcacaagagtccgcccccggtgaccctcacggatggcca |
| | | cacaaaataattcaacactactatcaacgccacccagtatacactagcttg |
| | | gctgtagcagcttccgctgtggcggtgatagtcggcctggccaccgccgtg |
| | | atgtgtatctgcagagcgcgacgggagtgcctaaccccgtacgcgttagcc |
| | | ccgaatgcagtcatcccgacgtccttggccttattgtgctgcgttcggtcc |
| | | gccaacgccgaatctttcaccgacacgatgggatacctatggtcgaacagt |
| | | caaacatttttctgggtgcagctgtgcatccccttggcagtagtaatagta |
| | | ctcgtacgctgctgttcgtgctgcctcccttttttagtggttgcaggcgcc |
| | | tacctggcgaaggtagacgccttcgaacatgcgaccactgtcccaaatgtg |
| | | ccgaagattccgtataaagctttggtcgagagagccggatacgccccgctt |
| | | aacttggaaattactgttatgtcttcagaggtcctcccttcgattcaccaa |
| | | gaatacatcacctgcaaatactccactatcgtgccttccccgaaagtgaaa |
| | | tgctgcggttcccttgagtgccaacccgcttcgcatgccgactacaactgc |
| | | aaagtatttggcggcgtttaccctttcatgtggggtggagcgcaatgtttt |
| | | tgcgacagcgagaacagccagatgagtgaggcgtatgtcgagctatcggcc |
| | | gattgtgcccttgatcacgcacaggcggtgaaagtgcacacagccgcaatg |
| | | aaggtcggactacgcattgtgtatgggaacaccactagcctcctggatgcg |
| | | tacgtaaacggagtcacgccaggcacatctaaagacctgaaagtcattgcc |
| | | ggacccatctcgtccgcgttttccccttttgactctaaagtcgtgatacac |
| | | cgagggctcgtgtacaattacgattttccagagtacggggcaatgaaaccg |
| | | ggtgcctttggggacattcaagctagctctttgacgggcaacgacctgatt |
| | | gccagcaccgacatacgtttgcttaaacccgcagcaaagaatgtgcacgtt |
| | | ccctatacccaagcagcatcaggatttgaaatttggaagaacaactccgga |
| | | agaccactgcaagagacggccccatttggctgtaagattgccgtcaaccct |
| | | ctgaaggccgtggattgttcctatgggaacatcccgatatcgattgatatc |
| | | ccccacgccgcctttattagaccatccgatgcgccatttatctccgaagtg |
| | | aagtgcgaagtcagcgactgcacatactctgctgacttcggaggcatggcc |
| | | accctacagtatgtgtctgaccgagagggccagtgcccagtgcattcccat |
| | | tcaagtaccgccacactccaggaatcaactgttcacgtcttggaaaaaggg |
| | | gcgacgacagtgcacttcagcacagctagccctcacgccagctttgtggtc |
| | | tcattatgcggaaagaagacgacctgcaccgcaggatgcaaaccacctgtc |
| | | ggccacatagtcagcacaccccacaagaaggaccaagagttccagaccgcc |
| | | gtctccaggacatcatggagctggctcttcgcgctgttcggaggagcttcg |
| | | tccctagtaatcatgggactgttaatcttctcctgcagcatgatgctcact |
| | | aacaccagaagatga |
| Nucleic acid | 58 | aaaaaaaaaacgcgtgataacgcaggaaagaacatgtgagcaaaaggccag |
| template, | | caaaaggccaggaaccgtaaaaaggccgcgttgctggcgtttttccatagg |
| SAM001 | | ctccgcccccctgacgagcatcacaaaaatcgacgctcaagtcagaggtgg |
| | | cgaaacccgacaggactataaagataccaggcgtttccccctggaagctcc |
| | | ctcgtgcgctctcctgttccgaccctgccgcttaccggatacctgtccgcc |
| | | tttctcccttcgggaagcgtggcgctttctcatagctcacgctgtaggtat |
| | | ctcagttcggtgtaggtcgttcgctccaagctgggctgtgtgcacgaaccc |
| | | cccgttcagcccgaccgctgcgccttatccggtaactatcgtcttgagtcc |
| | | aacccggtaagacacgacttatcgccactggcagcagccactggtaacagg |
| | | attagcagagcgaggtatgtaggcggtgctacagagttcttgaagtggtgg |
| | | cctaactacggctacactagaagaacagtatttggtatctgcgctctgctg |
| | | aagccagttaccttcggaaaaagagttggtagctcttgatccggcaaacaa |
| | | accaccgctggtagcggtttttttgtttgcaagcagcagattacgcgcaga |
| | | aaaaaaggatctcaagaagatcctttgatcttttctacggggtctgacgct |
| | | cagtggaacgaaaactcacgttaagggattttggtcatgagattatcaaaa |
| | | aggatcttcacctagatccttttaaattaaaaatgaagttttaaatcaatc |
| | | taaagtatatnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnncacatttccccgaaaagtgcca |
| | | cctgagctctaatacgactcactataggataggcggcgcatgagagaagcc |
| | | cagaccaattacctacccaaaatggagaaagttcacgttgacatcgaggaa |
| | | gacagcccattcctcagagctttgcagcggagcttcccgcagtttgaggta |
| | | gaagccaagcaggtcactgataatgaccatgctaatgccagagcgttttcg |
| | | catctggcttcaaaactgatcgaaacggaggtggacccatccgacacgatc |
| | | cttgacattggaagtgcgcccgcccgcagaatgtattctaagcacaagtat |
| | | cattgtatctgtccgatgagatgtgcggaagatccggacagattgtataag |
| | | tatgcaactaagctgaagaaaaactgtaaggaaataactgataaggaattg |
| | | gacaagaaaatgaaggagctggccgccgtcatgagcgaccctgacctggaa |
| | | actgagactatgtgcctccacgacgacgagtcgtgtcgctacgaagggcaa |
| | | gtcgctgtttaccaggatgtatacgcggttgacggaccgacaagtctctat |
| | | caccaagccaataagggagttagagtcgcctactggataggctttgacacc |
| | | accccttttatgtttaagaacttggctggagcatatccatcatactctacc |
| | | aactgggccgacgaaaccgtgttaacggctcgtaacataggcctatgcagc |
| | | tctgacgttatggagcggtcacgtagagggatgtccattcttagaaagaag |
| | | tatttgaaaccatccaacaatgttctattctctgttggctcgaccatctac |
| | | cacgagaagagggacttactgaggagctggcacctgccgtctgtatttcac |
| | | ttacgtggcaagcaaaattacacatgtcggtgtgagactatagttagttgc |
| | | gacgggtacgtcgttaaaagaatagctatcagtccaggcctgtatgggaag |
| | | ccttcaggctatgctgctacgatgcaccgcgagggattcttgtgctgcaaa |
| | | gtgacagacacattgaacggggagagggtctcttttcccgtgtgcacgtat |
| | | gtgccagctacattgtgtgaccaaatgactggcatactggcaacagatgtc |
| | | agtgcggacgacgcgcaaaaactgctggttgggctcaaccagcgtatagtc |
| | | gtcaacggtcgcacccagagaaacaccaataccatgaaaaattaccttttg |
| | | cccgtagtggcccaggcatttgctaggtgggcaaaggaatataaggaagat |
| | | caagaagatgaaaggccactaggactacgagatagacagttagtcatgggg |
| | | tgttgttgggcttttagaaggcacaagataacatctatttataagcgcccg |
| | | gatacccaaaccatcatcaaagtgaacagcgatttccactcattcgtgctg |
| | | cccaggataggcagtaacacattggagatcgggctgagaacaagaatcagg |
| | | aaaatgttagaggagcacaaggagccgtcacctctcattaccgccgaggac |
| | | gtacaagaagctaagtgcgcagccgatgaggctaaggaggtgcgtgaagcc |
| | | gaggagttgcgcgcagctctaccacctttggcagctgatgttgaggagccc |
| | | actctggaagccgatgtcgacttgatgttacaagaggctggggccggctca |
| | | gtggagacacctcgtggcttgataaaggttaccagctacgatggcgaggac |
| | | aagatcggctcttacgctgtgctttctccgcaggctgtactcaagagtgaa |
| | | aaattatcttgcatccaccctctcgctgaacaagtcatagtgataacacac |
| | | tctggccgaaaagggcgttatgccgtggaaccataccatggtaaagtagtg |
| | | gtgccagagggacatgcaatacccgtccaggactttcaagctctgagtgaa |
| | | agtgccaccattgtgtacaacgaacgtgagttcgtaaacaggtacctgcac |
| | | catattgccacacatggaggagcgctgaacactgatgaagaatattacaaa |
| | | actgtcaagcccagcgagcacgacggcgaatacctgtacgacatcgacagg |
| | | aaacagtgcgtcaagaaagaactagtcactgggctagggctcacaggcgag |
| | | ctggtggatcctcccttccatgaattcgcctacgagagtctgagaacacga |
| | | ccagccgctccttaccaagtaccaaccataggggtgtatggcgtgccagga |
| | | tcaggcaagtctggcatcattaaaagcgcagtcaccaaaaaagatctagtg |
| | | gtgagcgccaagaaagaaaactgtgcagaaattataagggacgtcaagaaa |
| | | atgaaagggctggacgtcaatgccagaactgtggactcagtgctcttgaat |
| | | ggatgcaaacaccccgtagagaccctgtatattgacgaagcttttgcttgt |
| | | catgcaggtactctcagagcgctcatagccattataagacctaaaaaggca |
| | | gtgctctgcggggatcccaaacagtgcggtttttttaacatgatgtgcctg |
| | | aaagtgcattttaaccacgagatttgcacacaagtcttccacaaaagcatc |
| | | tctcgccgttgcactaaatctgtgacttcggtcgtctcaaccttgttttac |
| | | gacaaaaaaatgagaacgacgaatccgaaagagactaagattgtgattgac |
| | | actaccggcagtaccaaacctaagcaggacgatctcattctcacttgtttc |
| | | agagggtgggtgaagcagttgcaaatagattacaaaggcaacgaaataatg |
| | | acggcagctgcctctcaagggctgacccgtaaaggtgtgtatgccgttcgg |
| | | tacaaggtgaatgaaaatcctctgtacgcacccacctcagaacatgtgaac |
| | | gtcctactgacccgcacggaggaccgcatcgtgtggaaaacactagccggc |
| | | gacccatggataaaaacactgactgccaagtaccctgggaatttcactgcc |
| | | acgatagaggagtggcaagcagagcatgatgccatcatgaggcacatcttg |
| | | gagagaccggaccctaccgacgtcttccagaataaggcaaacgtgtgttgg |
| | | gccaaggctttagtgccggtgctgaagaccgctggcatagacatgaccact |
| | | gaacaatggaacactgtggattattttgaaacggacaaagctcactcagca |
| | | gagatagtattgaaccaactatgcgtgaggttctttggactcgatctggac |
| | | tccggtctattttctgcacccactgttccgttatccattaggaataatcac |
| | | tgggataactccccgtcgcctaacatgtacgggctgaataaagaagtggtc |
| | | cgtcagctctctcgcaggtacccacaactgcctcgggcagttgccactgga |
| | | agagtctatgacatgaacactggtacactgcgcaattatgatccgcgcata |
| | | aacctagtacctgtaaacagaagactgcctcatgctttagtcctccaccat |
| | | aatgaacacccacagagtgacttttcttcattcgtcagcaaattgaagggc |
| | | agaactgtcctggtggtcggggaaaagttgtccgtcccaggcaaaatggtt |
| | | gactggttgtcagaccggcctgaggctaccttcagagctcggctggattta |
| | | ggcatcccaggtgatgtgcccaaatatgacataatatttgttaatgtgagg |
| | | accccatataaataccatcactatcagcagtgtgaagaccatgccattaag |
| | | cttagcatgttgaccaagaaagcttgtctgcatctgaatcccggcggaacc |
| | | tgtgtcagcataggttatggttacgctgacagggccagcgaaagcatcatt |
| | | ggtgctatagcgcggcagttcaagttttcccgggtatgcaaaccgaaatcc |
| | | tcacttgaagagacggaagttctgtttgtattcattgggtacgatcgcaag |
| | | gcccgtacgcacaatccttacaagctttcatcaaccttgaccaacatttat |
| | | acaggttccagactccacgaagccggatgtgcaccctcatatcatgtggtg |
| | | cgaggggatattgccacggccaccgaaggagtgattataaatgctgctaac |
| | | agcaaaggacaacctggcggaggggtgtgcggagcgctgtataagaaattc |
| | | ccggaaagcttcgatttacagccgatcgaagtaggaaaagcgcgactggtc |
| | | aaaggtgcagctaaacatatcattcatgccgtaggaccaaacttcaacaaa |
| | | gtttcggaggttgaaggtgacaaacagttggcagaggcttatgagtccatc |
| | | gctaagattgtcaacgataacaattacaagtcagtagcgattccactgttg |
| | | tccaccggcatcttttccgggaacaaagatcgactaacccaatcattgaac |
| | | catttgctgacagctttagacaccactgatgcagatgtagccatatactgc |
| | | agggacaagaaatgggaaatgactctcaaggaagcagtggctaggagagaa |
| | | gcagtggaggagatatgcatatccgacgactcttcagtgacagaacctgat |
| | | gcagagctggtgagggtgcatccgaagagttctttggctggaaggaagggc |
| | | tacagcacaagcgatggcaaaactttctcatatttggaagggaccaagttt |
| | | caccaggcggccaaggatatagcagaaattaatgccatgtggcccgttgca |
| | | acggaggccaatgagcaggtatgcatgtatatcctcggagaaagcatgagc |
| | | agtattaggtcgaaatgccccgtcgaagagtcggaagcctccacaccacct |
| | | agcacgctgccttgcttgtgcatccatgccatgactccagaaagagtacag |
| | | cgcctaaaagcctcacgtccagaacaaattactgtgtgctcatcctttcca |
| | | ttgccgaagtatagaatcactggtgtgcagaagatccaatgctcccagcct |
| | | atattgttctcaccgaaagtgcctgcgtatattcatccaaggaagtatctc |
| | | gtggaaacaccaccggtagacgagactccggagccatcggcagagaaccaa |
| | | tccacagaggggacacctgaacaaccaccacttataaccgaggatgagacc |
| | | aggactagaacgcctgagccgatcatcatcgaagaggaagaagaggatagc |
| | | ataagtttgctgtcagatggcccgacccaccaggtgctgcaagtcgaggca |
| | | gacattcacgggccgccctctgtatctagctcatcctggtccattcctcat |
| | | gcatccgactttgatgtggacagtttatccatacttgacaccctggaggga |
| | | gctagcgtgaccagcggggcaacgtcagccgagactaactcttacttcgca |
| | | aagagtatggagtttctggcgcgaccggtgcctgcgcctcgaacagtattc |
| | | aggaaccctccacatcccgctccgcgcacaagaacaccgtcacttgcaccc |
| | | agcagggcctgctcgagaaccagcctagtttccaccccgccaggcgtgaat |
| | | agggtgatcactagagaggagctcgaggcgcttaccccgtcacgcactcct |
| | | agcaggtcggtctcgagaaccagcctggtctccaacccgccaggcgtaaat |
| | | agggtgattacaagagaggagtttgaggcgttcgtagcacaacaacaatga |
| | | cggtttgatgcgggtgcatacatcttttcctccgacaccggtcaagggcat |
| | | ttacaacaaaaatcagtaaggcaaacggtgctatccgaagtggtgttggag |
| | | aggaccgaattggagatttcgtatgccccgcgcctcgaccaagaaaaagaa |
| | | gaattactacgcaagaaattacagttaaatcccacacctgctaacagaagc |
| | | agataccagtccaggaaggtggagaacatgaaagccataacagctagacgt |
| | | attctgcaaggcctagggcattatttgaaggcagaaggaaaagtggagtgc |
| | | taccgaaccctgcatcctgttcctttgtattcatctagtgtgaaccgtgcc |
| | | ttttcaagccccaaggtcgcagtggaagcctgtaacgccatgttgaaagag |
| | | aactttccgactgtggcttcttactgtattattccagagtacgatgcctat |
| | | ttggacatggttgacggagcttcatgctgcttagacactgccagtttttgc |
| | | cctgcaaagctgcgcagctttccaaagaaacactcctatttggaacccaca |
| | | atacgatcggcagtgccttcagcgatccagaacacgctccagaacgtcctg |
| | | gcagctgccacaaaaagaaattgcaatgtcacgcaaatgagagaattgccc |
| | | gtattggattcggcggcctttaatgtggaatgcttcaagaaatatgcgtgt |
| | | aataatgaatattgggaaacgtttaaagaaaaccccatcaggcttactgaa |
| | | gaaaacgtggtaaattacattaccaaattaaaaggaccaaaagctgctgct |
| | | ctttttgcgaagacacataatttgaatatgttgcaggacataccaatggac |
| | | aggtttgtaatggacttaaagagagacgtgaaagtgactccaggaacaaaa |
| | | catactgaagaacggcccaaggtacaggtgatccaggctgccgatccgcta |
| | | gcaacagcgtatctgtgcggaatccaccgagagctggttaggagattaaat |
| | | gcggtcctgcttccgaacattcatacactgtttgatatgtcggctgaagac |
| | | tttgacgctattatagccgagcacttccagcctggggattgtgttctggaa |
| | | actgacatcgcgtcgtttgataaaagtgaggacgacgccatggctctgacc |
| | | gcgttaatgattctggaagacttaggtgtggacgcagagctgttgacgctg |
| | | attgaggcggctttcggcgaaatttcatcaatacatttgcccactaaaact |
| | | aaatttaaattcggagccatgatgaaatctggaatgttcctcacactgttt |
| | | gtgaacacagtcattaacattgtaatcgcaagcagagtgttgagagaacgg |
| | | ctaaccggatcaccatgtgcagcattcattggagatgacaatatcgtgaaa |
| | | ggagtcaaatcggacaaattaatggcagacaggtgcgccacctggttgaat |
| | | atggaagtcaagattatagatgctgtggtgggcgagaaagcgccttatttc |
| | | tgtggagggtttattttgtgtgactccgtgaccggcacagcgtgccgtgtg |
| | | gcagaccccctaaaaaggctgtttaagcttggcaaacctctggcagcagac |
| | | gatgaacatgatgatgacaggagaagggcattgcatgaagagtcaacacgc |
| | | tggaaccgagtgggtattctttcagagctgtgcaaggcagtagaatcaagg |
| | | tatgaaaccgtaggaacttccatcatagttatggccatgactactctagct |
| | | agcagtgttaaatcattcagctacctgagaggggcccctataactctctac |
| | | ggctaacctgaatggactacgacatagtctagtccgccaagttcgaaggcg |
| | | cgcctctagagccaccnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnaagtccgccatgcccgaaggctacgtccaggagcg |
| | | caccatcttcttcaaggacgacggcaactacaagacccgcgccgaggtgaa |
| | | gttcgagggcgacaccctggtgaaccgcatcgagctgaagggcatcgactt |
| | | caaggaggacggcaacatcctggggcacaagctggagtacaactacaacag |
| | | ccacaacgtctatatcatggccgacaagcagaagaacggcatcaaggtgaa |
| | | cttcaagatccgccacaacatcgaggacggcagcgtgcagctcgccgacca |
| | | ctaccagcagaacacccccatcggcgacggccccgtgctgctgcccgacaa |
| | | ccactacctgagcacccagtccgccctgagcaaagaccccaacgagaagcg |
| | | cgatcacatggtcctgctggagttcgtgaccgccgccgggatcactctcgg |
| | | catggacgagctgtacaagtagcatcgatgatatcgcggccgcatacagca |
| | | gcaattggcaagctgcttacatagaactcgcggcgattggcatgccgcctt |
| | | aaaatttttattttatttttcttttcttttccgaatcggattttgttttta |
| | | atatttcaaaaaaaaaaaaaaaa |
| Nucleic acid | 59 | aaaaaaaaaacgcgtgataacgcaggaaagaacatgtgagcaaaaggccag |
| template, | | caaaaggccaggaaccgtaaaaaggccgcgttgctggcgtttttccatagg |
| SAM002 | | ctccgcccccctgacgagcatcacaaaaatcgacgctcaagtcagaggtgg |
| | | cgaaacccgacaggactataaagataccaggcgtttccccctggaagctcc |
| | | ctcgtgcgctctcctgttccgaccctgccgcttaccggatacctgtccgcc |
| | | tttctcccttcgggaagcgtggcgctttctcatagctcacgctgtaggtat |
| | | ctcagttcggtgtaggtcgttcgctccaagctgggctgtgtgcacgaaccc |
| | | cccgttcagcccgaccgctgcgccttatccggtaactatcgtcttgagtcc |
| | | aacccggtaagacacgacttatcgccactggcagcagccactggtaacagg |
| | | attagcagagcgaggtatgtaggcggtgctacagagttcttgaagtggtgg |
| | | cctaactacggctacactagaagaacagtatttggtatctgcgctctgctg |
| | | aagccagttaccttcggaaaaagagttggtagctcttgatccggcaaacaa |
| | | accaccgctggtagcggtttttttgtttgcaagcagcagattacgcgcaga |
| | | aaaaaaggatctcaagaagatcctttgatcttttctacggggtctgacgct |
| | | cagtggaacgaaaactcacgttaagggattttggtcatgagattatcaaaa |
| | | aggatcttcacctagatccttttaaattaaaaatgaagttttaaatcaatc |
| | | taaagtatatnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnncacatttccccgaaaagtgcca |
| | | cctgagctctaatacgactcactataggataggcggcgcatgagagaagcc |
| | | cagaccaattacctacccaaaatggagaaagttcacgttgacatcgaggaa |
| | | gacagcccattcctcagagctttgcagcggagcttcccgcagtttgaggta |
| | | gaagccaagcaggtcactgataatgaccatgctaatgccagagcgttttcg |
| | | catctggcttcaaaactgatcgaaacggaggtggacccatccgacacgatc |
| | | cttgacattggaagtgcgcccgcccgcagaatgtattctaagcacaagtat |
| | | cattgtatctgtccgatgagatgtgcggaagatccggacagattgtataag |
| | | tatgcaactaagctgaagaaaaactgtaaggaaataactgataaggaattg |
| | | gacaagaaaatgaaggagctggccgccgtcatgagcgaccctgacctggaa |
| | | actgagactatgtgcctccacgacgacgagtcgtgtcgctacgaagggcaa |
| | | gtcgctgtttaccaggatgtatacgcggttgacggaccgacaagtctctat |
| | | caccaagccaataagggagttagagtcgcctactggataggctttgacacc |
| | | accccttttatgtttaagaacttggctggagcatatccatcatactctacc |
| | | aactgggccgacgaaaccgtgttaacggctcgtaacataggcctatgcagc |
| | | tctgacgttatggagcggtcacgtagagggatgtccattcttagaaagaag |
| | | tatttgaaaccatccaacaatgttctattctctgttggctcgaccatctac |
| | | cacgagaagagggacttactgaggagctggcacctgccgtctgtatttcac |
| | | ttacgtggcaagcaaaattacacatgtcggtgtgagactatagttagttgc |
| | | gacgggtacgtcgttaaaagaatagctatcagtccaggcctgtatgggaag |
| | | ccttcaggctatgctgctacgatgcaccgcgagggattcttgtgctgcaaa |
| | | gtgacagacacattgaacggggagagggtctcttttcccgtgtgcacgtat |
| | | gtgccagctacattgtgtgaccaaatgactggcatactggcaacagatgtc |
| | | agtgcggacgacgcgcaaaaactgctggttgggctcaaccagcgtatagtc |
| | | gtcaacggtcgcacccagagaaacaccaataccatgaaaaattaccttttg |
| | | cccgtagtggcccaggcatttgctaggtgggcaaaggaatataaggaagat |
| | | caagaagatgaaaggccactaggactacgagatagacagttagtcatgggg |
| | | tgttgttgggcttttagaaggcacaagataacatctatttataagcgcccg |
| | | gatacccaaaccatcatcaaagtgaacagcgatttccactcattcgtgctg |
| | | cccaggataggcagtaacacattggagatcgggctgagaacaagaatcagg |
| | | aaaatgttagaggagcacaaggagccgtcacctctcattaccgccgaggac |
| | | gtacaagaagctaagtgcgcagccgatgaggctaaggaggtgcgtgaagcc |
| | | gaggagttgcgcgcagctctaccacctttggcagctgatgttgaggagccc |
| | | actctggaagccgatgtcgacttgatgttacaagaggctggggccggctca |
| | | gtggagacacctcgtggcttgataaaggttaccagctacgatggcgaggac |
| | | aagatcggctcttacgctgtgctttctccgcaggctgtactcaagagtgaa |
| | | aaattatcttgcatccaccctctcgctgaacaagtcatagtgataacacac |
| | | tctggccgaaaagggcgttatgccgtggaaccataccatggtaaagtagtg |
| | | gtgccagagggacatgcaatacccgtccaggactttcaagctctgagtgaa |
| | | agtgccaccattgtgtacaacgaacgtgagttcgtaaacaggtacctgcac |
| | | catattgccacacatggaggagcgctgaacactgatgaagaatattacaaa |
| | | actgtcaagcccagcgagcacgacggcgaatacctgtacgacatcgacagg |
| | | aaacagtgcgtcaagaaagaactagtcactgggctagggctcacaggcgag |
| | | ctggtggatcctcccttccatgaattcgcctacgagagtctgagaacacga |
| | | ccagccgctccttaccaagtaccaaccataggggtgtatggcgtgccagga |
| | | tcaggcaagtctggcatcattaaaagcgcagtcaccaaaaaagatctagtg |
| | | gtgagcgccaagaaagaaaactgtgcagaaattataagggacgtcaagaaa |
| | | atgaaagggctggacgtcaatgccagaactgtggactcagtgctcttgaat |
| | | ggatgcaaacaccccgtagagaccctgtatattgacgaagcttttgcttgt |
| | | catgcaggtactctcagagcgctcatagccattataagacctaaaaaggca |
| | | gtgctctgcggggatcccaaacagtgcggtttttttaacatgatgtgcctg |
| | | aaagtgcattttaaccacgagatttgcacacaagtcttccacaaaagcatc |
| | | tctcgccgttgcactaaatctgtgacttcggtcgtctcaaccttgttttac |
| | | gacaaaaaaatgagaacgacgaatccgaaagagactaagattgtgattgac |
| | | actaccggcagtaccaaacctaagcaggacgatctcattctcacttgtttc |
| | | agagggtgggtgaagcagttgcaaatagattacaaaggcaacgaaataatg |
| | | acggcagctgcctctcaagggctgacccgtaaaggtgtgtatgccgttcgg |
| | | tacaaggtgaatgaaaatcctctgtacgcacccacctcagaacatgtgaac |
| | | gtcctactgacccgcacggaggaccgcatcgtgtggaaaacactagccggc |
| | | gacccatggataaaaacactgactgccaagtaccctgggaatttcactgcc |
| | | acgatagaggagtggcaagcagagcatgatgccatcatgaggcacatcttg |
| | | gagagaccggaccctaccgacgtcttccagaataaggcaaacgtgtgttgg |
| | | gccaaggctttagtgccggtgctgaagaccgctggcatagacatgaccact |
| | | gaacaatggaacactgtggattattttgaaacggacaaagctcactcagca |
| | | gagatagtattgaaccaactatgcgtgaggttctttggactcgatctggac |
| | | tccggtctattttctgcacccactgttccgttatccattaggaataatcac |
| | | tgggataactccccgtcgcctaacatgtacgggctgaataaagaagtggtc |
| | | cgtcagctctctcgcaggtacccacaactgcctcgggcagttgccactgga |
| | | agagtctatgacatgaacactggtacactgcgcaattatgatccgcgcata |
| | | aacctagtacctgtaaacagaagactgcctcatgctttagtcctccaccat |
| | | aatgaacacccacagagtgacttttcttcattcgtcagcaaattgaagggc |
| | | agaactgtcctggtggtcggggaaaagttgtccgtcccaggcaaaatggtt |
| | | gactggttgtcagaccggcctgaggctaccttcagagctcggctggattta |
| | | ggcatcccaggtgatgtgcccaaatatgacataatatttgttaatgtgagg |
| | | accccatataaataccatcactatcagcagtgtgaagaccatgccattaag |
| | | cttagcatgttgaccaagaaagcttgtctgcatctgaatcccggcggaacc |
| | | tgtgtcagcataggttatggttacgctgacagggccagcgaaagcatcatt |
| | | ggtgctatagcgcggcagttcaagttttcccgggtatgcaaaccgaaatcc |
| | | tcacttgaagagacggaagttctgtttgtattcattgggtacgatcgcaag |
| | | gcccgtacgcacaattcttacaagctttcatcaaccttgaccaacatttat |
| | | acaggttccagactccacgaagccggatgtgcaccctcatatcatgtggtg |
| | | cgaggggatattgccacggccaccgaaggagtgattataaatgctgctaac |
| | | agcaaaggacaacctggcggaggggtgtgcggagcgctgtataagaaattc |
| | | ccggaaagcttcgatttacagccgatcgaagtaggaaaagcgcgactggtc |
| | | aaaggtgcagctaaacatatcattcatgccgtaggaccaaacttcaacaaa |
| | | gtttcggaggttgaaggtgacaaacagttggcagaggcttatgagtccatc |
| | | gctaagattgtcaacgataacaattacaagtcagtagcgattccactgttg |
| | | tccaccggcatcttttccgggaacaaagatcgactaacccaatcattgaac |
| | | catttgctgacagctttagacaccactgatgcagatgtagccatatactgc |
| | | agggacaagaaatgggaaatgactctcaaggaagcagtggctaggagagaa |
| | | gcagtggaggagatatgcatatccgacgactcttcagtgacagaacctgat |
| | | gcagagctggtgagggtgcatccgaagagttctttggctggaaggaagggc |
| | | tacagcacaagcgatggcaaaactttctcatatttggaagggaccaagttt |
| | | caccaggcggccaaggatatagcagaaattaatgccatgtggcccgttgca |
| | | acggaggccaatgagcaggtatgcatgtatatcctcggagaaagcatgagc |
| | | agtattaggtcgaaatgccccgtcgaagagtcggaagcctccacaccacct |
| | | agcacgctgccttgcttgtgcatccatgccatgactccagaaagagtacag |
| | | cgcctaaaagcctcacgtccagaacaaattactgtgtgctcatcctttcca |
| | | ttgccgaagtatagaatcactggtgtgcagaagatccaatgctcccagcct |
| | | atattgttctcaccgaaagtgcctgcgtatattcatccaaggaagtatctc |
| | | gtggaaacaccaccggtagacgagactccggagccatcggcagagaaccaa |
| | | tccacagaggggacacctgaacaaccaccacttataaccgaggatgagacc |
| | | aggactagaacgcctgagccgatcatcatcgaagaggaagaagaggatagc |
| | | ataagtttgctgtcagatggcccgacccaccaggtgctgcaagtcgaggca |
| | | gacattcacgggccgccctctgtatctagctcatcctggtccattcctcat |
| | | gcatccgactttgatgtggacagtttatccatacttgacaccctggaggga |
| | | gctagcgtgaccagcggggcaacgtcagccgagactaactcttacttcgca |
| | | aagagtatggagtttctggcgcgaccggtgcctgcgcctcgaacagtattc |
| | | aggaaccctccacatcccgctccgcgcacaagaacaccgtcacttgcaccc |
| | | agcagggcctgctcgagaaccagcctagtttccaccccgccaggcgtgaat |
| | | agggtgatcactagagaggagctcgaggcgcttaccccgtcacgcactcct |
| | | agcaggtcggtctcgagaaccagcctggtctccaacccgccaggcgtaaat |
| | | agggtgattacaagagaggagtttgaggcgttcgtagcacaacaacaatga |
| | | cggtttgatgcgggtgcatacatcttttcctccgacaccggtcaagggcat |
| | | ttacaacaaaaatcagtaaggcaaacggtgctatccgaagtggtgttggag |
| | | aggaccgaattggagatttcgtatgccccgcgcctcgaccaagaaaaagaa |
| | | gaattactacgcaagaaattacagttaaatcccacacctgctaacagaagc |
| | | agataccagtccaggaaggtggagaacatgaaagccataacagctagacgt |
| | | attctgcaaggcctagggcattatttgaaggcagaaggaaaagtggagtgc |
| | | taccgaaccctgcatcctgttcctttgtattcatctagtgtgaaccgtgcc |
| | | ttttcaagccccaaggtcgcagtggaagcctgtaacgccatgttgaaagag |
| | | aactttccgactgtggcttcttactgtattattccagagtacgatgcctat |
| | | ttggacatggttgacggagcttcatgctgcttagacactgccagtttttgc |
| | | cctgcaaagctgcgcagctttccaaagaaacactcctatttggaacccaca |
| | | atacgatcggcagtgccttcagcgatccagaacacgctccagaacgtcctg |
| | | gcagctgccacaaaaagaaattgcaatgtcacgcaaatgagagaattgccc |
| | | gtattggattcggcggcctttaatgtggaatgcttcaagaaatatgcgtgt |
| | | aataatgaatattgggaaacgtttaaagaaaaccccatcaggcttactgaa |
| | | gaaaacgtggtaaattacattaccaaattaaaaggaccaaaagctgctgct |
| | | ctttttgcgaagacacataatttgaatatgttgcaggacataccaatggac |
| | | aggtttgtaatggacttaaagagagacgtgaaagtgactccaggaacaaaa |
| | | catactgaagaacggcccaaggtacaggtgatccaggctgccgatccgcta |
| | | gcaacagcgtatctgtgcggaatccaccgagagctggttaggagattaaat |
| | | gcggtcctgcttccgaacattcatacactgtttgatatgtcggctgaagac |
| | | tttgacgctattatagccgagcacttccagcctggggattgtgttctggaa |
| | | actgacatcgcgtcgtttgataaaagtgaggacgacgccatggctctgacc |
| | | gcgttaatgattctggaagacttaggtgtggacgcagagctgttgacgctg |
| | | attgaggcggctttcggcgaaatttcatcaatacatttgcccactaaaact |
| | | aaatttaaattcggagccatgatgaaatctggaatgttcctcacactgttt |
| | | gtgaacacagtcattaacattgtaatcgcaagcagagtgttgagagaacgg |
| | | ctaaccggatcaccatgtgcagcattcattggagatgacaatatcgtgaaa |
| | | ggagtcaaatcggacaaattaatggcagacaggtgcgccacctggttgaat |
| | | atggaagtcaagattatagatgctgtggtgggcgagaaagcgccttatttc |
| | | tgtggagggtttattttgtgtgactccgtgaccggcacagcgtgccgtgtg |
| | | gcagaccccctaaaaaggctgtttaagcttggcaaacctctggcagcagac |
| | | gatgaacatgatgatgacaggagaagggcattgcatgaagagtcaacacgc |
| | | tggaaccgagtgggtattctttcagagctgtgcaaggcagtagaatcaagg |
| | | tatgaaaccgtaggaacttccatcatagttatggccatgactactctagct |
| | | agcagtgttaaatcattcagctacctgagaggggcccctataactctctac |
| | | ggctaacctgaatggactacgacatagtctagtccgccaagttcgaaggcg |
| | | cgcctctagagccaccnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnnnnnnnnnnnnnnnnnnnnnnnnnnnnnnnnnnnn |
| | | nnnnnnnnnnnnnnnnaagtccgccatgcccgaaggctacgtccaggagcg |
| | | caccatcttcttcaaggacgacggcaactacaagacccgcgccgaggtgaa |
| | | gttcgagggcgacaccctggtgaaccgcatcgagctgaagggcatcgactt |
| | | caaggaggacggcaacatcctggggcacaagctggagtacaactacaacag |
| | | ccacaacgtctatatcatggccgacaagcagaagaacggcatcaaggtgaa |
| | | cttcaagatccgccacaacatcgaggacggcagcgtgcagctcgccgacca |
| | | ctaccagcagaacacccccatcggcgacggccccgtgctgctgcccgacaa |
| | | ccactacctgagcacccagtccgccctgagcaaagaccccaacgagaagcg |
| | | cgatcacatggtcctgctggagttcgtgaccgccgccgggatcactctcgg |
| | | catggacgagctgtacaagtagcatcgatgatatcgcggccgcatacagca |
| | | gcaattggcaagctgcttacatagaactcgcggcgattggcatgccgcctt |
| | | aaaatttttattttatttttcttttcttttccgaatcggattttgttttta |
| | | atatttcaaaaaaaaaaaaaaaa |
| Contig 1 | 60 | atggagaaagttcacgttgacatcgaggaagacagcccattcctcagagct |
| | | ttgcagcggagcttcccgcagtttgaggtagaagccaagcaggtcactgat |
| | | aatgaccatgctaatgccagagcgttttcgcatctggcttcaaaactgatc |
| | | gaaacggaggtggacccatccgacacgatccttgacattggaagtgcgccc |
| | | gcccgcagaatgtattctaagcacaagtatcattgtatctgtccgatgaga |
| | | tgtgcggaagatccggacagattgtataagtatgcaactaagctgaagaaa |
| | | aactgtaaggaaataactgataaggaattggacaagaaaatgaaggagctg |
| | | gccgccgtcatgagcgaccctgacctggaaactgagactatgtgcctccac |
| | | gacgacgagtcgtgtcgctacgaagggcaagtcgctgtttaccaggatgta |
| | | tacgcggttgacggaccgacaagtctctatcaccaagccaataagggagtt |
| | | agagtcgcctactggataggctttgacaccaccccttttatgtttaagaac |
| | | ttggctggagcatatccatcatactctaccaactgggccgacgaaaccgtg |
| | | ttaacggctcgtaacataggcctatgcagctctgacgttatggagcggtca |
| | | cgtagagggatgtccattcttagaaagaagtatttgaaaccatccaacaat |
| | | gttctattctctgttggctcgaccatctaccacgagaagagggacttactg |
| | | aggagctggcacctgccgtctgtatttcacttacgtggcaagcaaaattac |
| | | acatgtcggtgtgagactatagttagttgcgacgggtacgtcgttaaaaga |
| | | atagctatcagtccaggcctgtatgggaagccttcaggctatgctgctacg |
| | | atgcaccgcgagggattcttgtgctgcaaagtgacagacacattgaacggg |
| | | gagagggtctcttttcccgtgtgcacgtatgtgccagctacattgtgtgac |
| | | caaatgactggcatactggcaacagatgtcagtgcggacgacgcgcaaaaa |
| | | ctgctggttgggctcaaccagcgtatagtcgt |
| Contig 2 | 61 | tggcaacagatgtcagtgcggacgacgcgcaaaaactgct |
| | | ggttgggctcaaccagcgtatagtcgtcaacggtcgcacccagagaaacac |
| | | caataccatgaaaaattaccttttgcccgtagtggcccaggcatttgctag |
| | | gtgggcaaaggaatataaggaagatcaagaagatgaaaggccactaggact |
| | | acgagatagacagttagtcatggggtgttgttgggcttttagaaggcacaa |
| | | gataacatctatttataagcgcccggatacccaaaccatcatcaaagtgaa |
| | | cagcgatttccactcattcgtgctgcccaggataggcagtaacacattgga |
| | | gatcgggctgagaacaagaatcaggaaaatgttagaggagcacaaggagcc |
| | | gtcacctctcattaccgccgaggacgtacaagaagctaagtgcgcagccga |
| | | tgaggctaaggaggtgcgtgaagccgaggagttgcgcgcagctctaccacc |
| | | tttggcagctgatgttgaggagcccactctggaagccgatgtcgacttgat |
| | | gttacaagaggctggggccggctcagtggagacacctcgtggcttgataaa |
| | | ggttaccagctacgatggcgaggacaagatcggctcttacgctgtgctttc |
| | | tccgcaggctgtactcaagagtgaaaaattatcttgcatccaccctctcgc |
| | | tgaacaagtcatagtgataacacactctggccgaaaagggcgttatgccgt |
| | | ggaaccataccatggtaaagtagtggtgccagagggacatgcaatacccgt |
| | | ccaggactttcaagctctgagtgaaagtgccaccattgtgtacaacgaacg |
| | | tgagttcgtaaacaggtacctgcaccatattgccacacatggaggagcgct |
| | | gaacactgatgaagaatattacaaaactgtcaagcccagcgagcacgacgg |
| | | cgaatacctgtacgacatcgacaggaaacagtgcgtcaagaaagaactagt |
| | | cactgggctagggctcacaggcgagctggtggatcctcccttccatgaatt |
| | | cgcctacgagagtctgagaacacgaccagccgctccttaccaagtaccaac |
| | | cataggggtgtatggcgtgccaggatcaggcaagtctggcatcatta |
| Contig 3 | 62 | taccaagtaccaaccataggggtgtatggcgtgccaggatcaggcaagtct |
| | | ggcatcattaaaagcgcagtcaccaaaaaagatctagtggtgagcgccaag |
| | | aaagaaaactgtgcagaaattataagggacgtcaagaaaatgaaagggctg |
| | | gacgtcaatgccagaactgtggactcagtgctcttgaatggatgcaaacac |
| | | cccgtagagaccctgtatattgacgaagcttttgcttgtcatgcaggtact |
| | | ctcagagcgctcatagccattataagacctaaaaaggcagtgctctgcggg |
| | | gatcccaaacagtgcggtttttttaacatgatgtgcctgaaagtgcatttt |
| | | aaccacgagatttgcacacaagtcttccacaaaagcatctctcgccgttgc |
| | | actaaatctgtgacttcggtcgtctcaaccttgttttacgacaaaaaaatg |
| | | agaacgacgaatccgaaagagactaagattgtgattgacactaccggcagt |
| | | accaaacctaagcaggacgatctcattctcacttgtttcagagggtgggtg |
| | | aagcagttgcaaatagattacaaaggcaacgaaataatgacggcagctgcc |
| | | tctcaagggctgacccgtaaaggtgtgtatgccgttcggtacaaggtgaat |
| | | gaaaatcctctgtacgcacccacctcagaacatgtgaacgtcctactgacc |
| | | cgcacggaggaccgcatcgtgtggaaaacactagccggcgacccatggata |
| | | aaaacactgactgccaagtaccctgggaatttcactgccacgatagaggag |
| | | tggcaagcagagcatgatgccatcatgaggcacatcttggagagaccggac |
| | | cctaccgacgtcttccagaataaggcaaacgtgtgttgggccaaggcttta |
| | | gtgccggtgctgaagaccgctggcatagacatgaccactgaacaatggaac |
| | | actgtggattattttgaaacggacaaagctcactcagcagagatagtattg |
| | | aaccaactatgcgtgaggttctttggactcgatctggactccggtctattt |
| | | tctgcacccactgttccgttatccattaggaataatcactgggataactcc |
| | | ccgtcgcctaacatgtacgggctgaa |
| Contig 4 | 63 | gaataatcactgggataactccccgtcgcctaacatgtacgggctgaataa |
| | | agaagtggtccgtcagctctctcgcaggtacccacaactgcctcgggcagt |
| | | tgccactggaagagtctatgacatgaacactggtacactgcgcaattatga |
| | | tccgcgcataaacctagtacctgtaaacagaagactgcctcatgctttagt |
| | | cctccaccataatgaacacccacagagtgacttttcttcattcgtcagcaa |
| | | attgaagggcagaactgtcctggtggtcggggaaaagttgtccgtcccagg |
| | | caaaatggttgactggttgtcagaccggcctgaggctaccttcagagctcg |
| | | gctggatttaggcatcccaggtgatgtgcccaaatatgacataatatttgt |
| | | taatgtgaggaccccatataaataccatcactatcagcagtgtgaagacca |
| | | tgccattaagcttagcatgttgaccaagaaagcttgtctgcatctgaatcc |
| | | cggcggaacctgtgtcagcataggttatggttacgctgacagggccagcga |
| | | aagcatcattggtgctatagcgcggcagttcaagttttcccgggtatgcaa |
| | | accgaaatcctcacttgaagagacggaagttctgtttgtattcattgggta |
| | | cgatcgcaaggcccgtacgcacaattcttacaagctttcatcaaccttgac |
| | | caacatttatacaggttccagactccacgaagccggatgtgcaccctcata |
| | | tcatgtggtgcgaggggatattgccacggccaccgaaggagtgattataaa |
| | | tgctgctaacagcaaaggacaacctggcggaggggtgtgcggagcgctgta |
| | | taagaaattcccggaaagcttcgatttacagccgatcgaagtaggaaaagc |
| | | gcgactggtcaaaggtgcagctaaacatatcattcatgccgtaggaccaaa |
| | | cttcaacaaagtttcggaggttgaaggtgacaaacagttggcagaggctta |
| | | tgagtccatcgctaagattgtcaacgataacaattacaagtcagtagcgat |
| | | tccactgttgtccaccggcatcttttccgggaacaaagatcgactaaccca |
| | | atcattgaaccatttgctgacagctttagacaccactgatgcagatg |
| Contig 5 | 64 | attgaaccatttgctgacagctttagacaccactgatgcagatgtagccat |
| | | atactgcagggacaagaaatgggaaatgactctcaaggaagcagtggctag |
| | | gagagaagcagtggaggagatatgcatatccgacgactcttcagtgacaga |
| | | acctgatgcagagctggtgagggtgcatccgaagagttctttggctggaag |
| | | gaagggctacagcacaagcgatggcaaaactttctcatatttggaagggac |
| | | caagtttcaccaggcggccaaggatatagcagaaattaatgccatgtggcc |
| | | cgttgcaacggaggccaatgagcaggtatgcatgtatatcctcggagaaag |
| | | catgagcagtattaggtcgaaatgccccgtcgaagagtcggaagcctccac |
| | | accacctagcacgctgccttgcttgtgcatccatgccatgactccagaaag |
| | | agtacagcgcctaaaagcctcacgtccagaacaaattactgtgtgctcatc |
| | | ctttccattgccgaagtatagaatcactggtgtgcagaagatccaatgctc |
| | | ccagcctatattgttctcaccgaaagtgcctgcgtatattcatccaaggaa |
| | | gtatctcgtggaaacaccaccggtagacgagactccggagccatcggcaga |
| | | gaaccaatccacagaggggacacctgaacaaccaccacttataaccgagga |
| | | tgagaccaggactagaacgcctgagccgatcatcatcgaagaggaagaaga |
| | | ggatagcataagtttgctgtcagatggcccgacccaccaggtgctgcaagt |
| | | cgaggcagacattcacgggccgccctctgtatctagctcatcctggtccat |
| | | tcctcatgcatccgactttgatgtggacagtttatccatacttgacaccct |
| | | ggagggagctagcgtgaccagcggggcaacgtcagccgagactaactctta |
| | | cttcgcaaagagtatggagtttctggcgcgaccggtgcctgcgcctcgaac |
| | | agtattcaggaaccctccacatcccgctccgcgcacaagaacaccgtcact |
| | | tgcacccagcagggcctgctcgagaaccagcctagtttccaccccgccagg |
| | | cgtgaatagggtgatcactagagagga |
| Contig 6 | 65 | agaaccagcctagtttccaccccgccaggcgtgaatagggtgatcactaga |
| | | gaggagctcgaggcgcttaccccgtcacgcactcctagcaggtcggtctcg |
| | | agaaccagcctggtctccaacccgccaggcgtaaatagggtgattacaaga |
| | | gaggagtttgaggcgttcgtagcacaacaacaatgacggtttgatgcgggt |
| | | gcatacatcttttcctccgacaccggtcaagggcatttacaacaaaaatca |
| | | gtaaggcaaacggtgctatccgaagtggtgttggagaggaccgaattggag |
| | | atttcgtatgccccgcgcctcgaccaagaaaaagaagaattactacgcaag |
| | | aaattacagttaaatcccacacctgctaacagaagcagataccagtccagg |
| | | aaggtggagaacatgaaagccataacagctagacgtattctgcaaggccta |
| | | gggcattatttgaaggcagaaggaaaagtggagtgctaccgaaccctgcat |
| | | cctgttcctttgtattcatctagtgtgaaccgtgccttttcaagccccaag |
| | | gtcgcagtggaagcctgtaacgccatgttgaaagagaactttccgactgtg |
| | | gcttcttactgtattattccagagtacgatgcctatttggacatggttgac |
| | | ggagcttcatgctgcttagacactgccagtttttgccctgcaaagctgcgc |
| | | agctttccaaagaaacactcctatttggaacccacaatacgatcggcagtg |
| | | ccttcagcgatccagaacacgctccagaacgtcctggcagctgccacaaaa |
| | | agaaattgcaatgtcacgcaaatgagagaattgcccgtattggattcggcg |
| | | gcctttaatgtggaatgcttcaagaaatatgcgtgtaataatgaatattgg |
| | | gaaacgtttaaagaaaaccccatcaggcttactgaagaaaacgtggtaaat |
| | | tacattaccaaattaaaaggaccaaaagctgctgctctttttgcgaagaca |
| | | cataatttgaatatgttgcaggacataccaatggacaggtttgtaatggac |
| | | ttaaagagagacgtgaaagtgactccaggaacaaaacatactgaagaacgg |
| | | cccaaggtacaggtgatccaggctgccgatccgctagcaacagcgtatctg |
| | | tgcggaatccaccgagagctggtta |
| Contig 7 | 66 | gctagcaacagcgtatctgtgcggaatccaccgagagctggttaggagatt |
| | | aaatgcggtcctgcttccgaacattcatacactgtttgatatgtcggctga |
| | | agactttgacgctattatagccgagcacttccagcctggggattgtgttct |
| | | ggaaactgacatcgcgtcgtttgataaaagtgaggacgacgccatggctct |
| | | gaccgcgttaatgattctggaagacttaggtgtggacgcagagctgttgac |
| | | gctgattgaggcggctttcggcgaaatttcatcaatacatttgcccactaa |
| | | aactaaatttaaattcggagccatgatgaaatctggaatgttcctcacact |
| | | gtttgtgaacacagtcattaacattgtaatcgcaagcagagtgttgagaga |
| | | acggctaaccggatcaccatgtgcagcattcattggagatgacaatatcgt |
| | | gaaaggagtcaaatcggacaaattaatggcagacaggtgcgccacctggtt |
| | | gaatatggaagtcaagattatagatgctgtggtgggcgagaaagcgcctta |
| | | tttctgtggagggtttattttgtgtgactccgtgaccggcacagcgtgccg |
| | | tgtggcagaccccctaaaaaggctgtttaagcttggcaaacctctggcagc |
| | | agacgatgaacatgatgatgacaggagaagggcattgcatgaagagtcaac |
| | | acgctggaaccgagtgggtattctttcagagctgtgcaaggcagtagaatc |
| | | aaggtatgaaaccgtaggaacttccatcatagttatggccatgactactct |
| | | agctagcagtgttaaatcattcagctacctgagaggggcccctataactct |
| | | ctacggctaacctgaatggactacgacatagtctagtccgccaagttcgaa |
| | | ggcgcgcctctagagccaccatgaccgagtacaagcccacggtgcgcctcg |
| | | ccacccgcgacgacgtccccagggccgtacgcaccctcgccgccgcgttcg |
| | | ccgactaccccgccacgcgccacaccgtcgatccggaccgccacatcgagc |
| | | gggtcaccgagctgcaagaactcttcct |
| Contig 8 | 67 | cacgcgcgtcgggctcgacatcggcaaggtgtgggtcgcggacgacggcgc |
| | | cgcggtggcggtctggaccacgccggagagcgtcgaagcgggggcggtgtt |
| | | cgccgagatcggcccgcgcatggccgagttgagcggttcccggctggccgc |
| | | gcagcaacagatggaaggcctcctggcgccgcaccggcccaaggagcccgc |
| | | gtggttcctggccaccgtcggcgtctcgcccgaccaccagggcaagggtct |
| | | gggcagcgccgtcgtgctccccggagtggaggcggccgagcgcgccggggt |
| | | gcccgccttcctggagacctccgcgccccgcaacctccccttctacgagcg |
| | | gctcggcttcaccgtcaccgccgacgtcgaggtgcccgaaggaccgcgcac |
| | | ctggtgcatgacccgcaagcccggtgcctgacatcgatgatatcgcggccg |
| | | catacagcagcaattggcaagctgcttacatagaactcgcggcgattggca |
| | | tgccgccttaaaatttttattttatttttcttttcttttccgaatcggatt |
| | | ttgtttttaatatttcaaaaaaaaaaaaaaaa |
| Selective | 68 | atgagtaaacttggtctgacagttaccaatgcttaatcagtgaggcaccta |
| marker, | | tctcagcgatctgtctatttcgttcatccatagttgcctgactccccgtcg |
| puromycin | | tgtagataactacgatacgggagggcttaccatctggccccagtgctgcaa |
| resistance gene | | tgataccgcgagacccacgctcaccggctccagatttatcagcaataaacc |
| | | agccagccggaagggccgagcgcagaagtggtcctgcaactttatccgcct |
| | | ccatccagtctattaattgttgccgggaagctagagtaagtagttcgccag |
| | | ttaatagtttgcgcaacgttgttgccattgctacaggcatcgtggtgtcac |
| | | gctcgtcgtttggtatggcttcattcagctccggttcccaacgatcaaggc |
| | | gagttacatgatcccccatgttgtgcaaaaaagcggttagctccttcggtc |
| | | ctccgatcgttgtcagaagtaagttggccgcagtgttatcactcatggtta |
| | | tggcagcactgcataattctcttactgtcatgccatccgtaagatgctttt |
| | | ctgtgactggtgagtactcaaccaagtcattctgagaatagtgtatgcggc |
| | | gaccgagttgctcttgcccggcgtcaatacgggataataccgcgccacata |
| | | gcagaactttaaaagtgctcatcattggaaaacgttcttcggggcgaaaac |
| | | tctcaaggatcttaccgctgttgagatccagttcgatgtaacccactcgtg |
| | | cacccaactgatcttcagcatcttttactttcaccagcgtttctgggtgag |
| | | caaaaacaggaaggcaaaatgccgcaaaaaagggaataagggcgacacgga |
| | | aatgttgaatactcatactcttcctttttcaatattattgaagcatttatc |
| | | agggttattgtctcatgagcggatacatatttgaatgtatttagaaaaata |
| | | aacaaataggggttccgcg |
| First nucleic | 73 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| human IL12 | | ggacccatccgacacgatccttgacattggaagtgcgcccgcccgcagaat |
| (Sam002- | | gtattctaagcacaagtatcattgtatctgtccgatgagatgtgcggaaga |
| hIL12) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatggactggacctggcgaata |
| | | ctgttcttggttgccgccgctacagggactcacgcaatatgggagctgaag |
| | | aaggacgtgtatgtcgtggagctggactggtacccagatgctcctggcgaa |
| | | atggtggttttaacatgtgatacccccgaggaggacggcatcacatggact |
| | | ctggaccagagttctgaggtgctggggtccggcaagactctgacaatccag |
| | | gttaaggagttcggcgacgcaggacagtacacttgtcacaagggaggtgag |
| | | gtgctttctcacagcctgttgctgctccataagaaggaagacggtatttgg |
| | | tcaaccgacatcctcaaggaccagaaggagcccaaaaacaagacctttctg |
| | | agatgtgaggccaagaattacagcggtagattcacttgttggtggctcacc |
| | | accatatccacagacttgaccttcagtgtcaaaagttcacgagggagctca |
| | | gatcctcaaggcgttacctgtggcgcagcgacgctgtccgcagaaagagtc |
| | | aggggagacaacaaggaatacgagtactctgtcgagtgccaggaggattcc |
| | | gcctgtccggccgcagaggagtctttacctattgaggtgatggtcgatgcc |
| | | gtgcacaagcttaagtacgagaattacacatcaagttttttcatccgcgac |
| | | atcattaaacctgatccaccaaagaacctgcagctcaagcctctgaagaat |
| | | agcaggcaggtcgaggtaagctgggagtatcctgatacctggtccaccccc |
| | | cacagttatttcagcctcaccttctgcgtccaagtccagggaaagagcaag |
| | | agagagaagaaggatagggtgttcacagataagacttcagctactgtgatc |
| | | tgcagaaagaatgcgtctatctctgtgcgagcacaagacaggtactacagt |
| | | tctagctggagcgagtgggcatcagtcccctgcagtggtggcggaagcgga |
| | | gggggcagcggaggtgggagcggagggagcaggaacctcccagttgctaca |
| | | cctgacccgggaatgtttccatgcctccaccattcccagaatctcctccga |
| | | gccgtgtccaatatgctgcaaaaggctcggcagaccttggagttttaccct |
| | | tgcacctcagaagaaatcgatcatgaggatatcacaaaggataagacgagc |
| | | actgttgaggcatgcctgcccctggagctaactaagaatgagtcttgcctg |
| | | aacagcagggagacttccttcattaccaacggtagctgtcttgccagcagg |
| | | aagacatcttttatgatggccctgtgtctatctagcatatatgaagacctg |
| | | aagatgtaccaggtggaattcaaaaccatgaatgctaagcttctcatggat |
| | | cccaagaggcaaatcttcctggaccagaatatgcttgctgtcatagatgaa |
| | | ctgatgcaggcgttgaattttaacagcgagacggtgcctcaaaaaagctca |
| | | ctggaagaacctgatttttataaaacgaagatcaagctgtgtattttacta |
| | | cacgcctttagaatccgcgctgttaccatcgacagagtaatgtcctaccta |
| | | aatgcttcatagcatcgatgatatcgcggccgcatacagcagcaattggca |
| | | agctgcttacatagaactcgcggcgattggcatgccgccttaaaattttta |
| | | ttttatttttcttttcttttccgaatcggattttgtttttaatatttcaaa |
| | | aaaaaaaaaaaaaaaaaaaaaaa |
| First nucleic | 74 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| human IL-15 | | ggacccatccgacacgatccttgacattggaagtgcgcccgcccgcagaat |
| (Sam002- | | gtattctaagcacaagtatcattgtatctgtccgatgagatgtgcggaaga |
| hIL15) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgtatcgaatgcaattgctg |
| | | tcctgcattgcgctctctctggcgcttgtcaccaattcaaactgggttaac |
| | | gtaatatctgatttgaaaaaaatcgaagacctcatacagagtatgcacata |
| | | gatgctacgctgtacacagaaagcgacgtacacccgagctgtaaggtcact |
| | | gccatgaagtgctttcttctcgaactccaagttatcagtctcgaatccggg |
| | | gatgcgtcaatccatgacactgttgagaatctcattattctcgcgaacaac |
| | | tcactcagctcaaatgggaacgtcactgaatccggctgcaaggaatgcgag |
| | | gaattggaagagaagaatattaaagagttcctgcagagtttcgttcatatt |
| | | gttcaaatgtttatcaacacaagttagcatcgatgatatcgcggccgcata |
| | | cagcagcaattggcaagctgcttacatagaactcgcggcgattggcatgcc |
| | | gccttaaaatttttattttatttttcttttcttttccgaatcggattttgt |
| | | ttttaatatttcaaaaaaaaaaaaaaaaaaaaaaaaaa |
| First nucleic | 75 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| mouse IL-15 | | ggacccatccgacacgatccttgacattggaagtgcgcccgcccgcagaat |
| (sam002- | | gtattctaagcacaagtatcattgtatctgtccgatgagatgtgcggaaga |
| mIL15) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgtattcaatgcagcttgcc |
| | | agttgtgtcacactcactcttgtcctgctggttaattccaattggatcgat |
| | | gtccggtatgatttggagaagatcgaatctctcatccaaagcatacacatt |
| | | gacaccactttgtatactgatagcgattttcacccatcttgtaaggtgacc |
| | | gccatgaattgctttctgttggaacttcaagttatacttcacgagtattca |
| | | aacatgacactgaatgaaactgtccgaaacgtactctaccttgctaactcc |
| | | accctttcatctaataagaacgtagctgagagcggatgtaaggagtgtgag |
| | | gagctcgaagagaagacatttactgaatttcttcagagttttatccgcata |
| | | gtgcaaatgtttattaacacctcctagcatcgatgatatcgcggccgcata |
| | | cagcagcaattggcaagctgcttacatagaactcgcggcgattggcatgcc |
| | | gccttaaaatttttattttatttttcttttcttttccgaatcggattttgt |
| | | ttttaatatttcaaaaaaaaaaaaaaaaaaaaaaaaaa |
| First nucleic | 76 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| human IL15 | | ggacccatccgacacgatccttgacattggaagtgcgcccgcccgcagaat |
| (Sam002- | | gtattctaagcacaagtatcattgtatctgtccgatgagatgtgcggaaga |
| hIL15) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgtatcgaatgcaattgctg |
| | | tcctgcattgcgctctctctggcgcttgtcaccaattcaaactgggttaac |
| | | gtaatatctgatttgaaaaaaatcgaagacctcatacagagtatgcacata |
| | | gatgctacgctgtacacagaaagcgacgtacacccgagctgtaaggtcact |
| | | gccatgaagtgctttcttctcgaactccaagttatcagtctcgaatccggg |
| | | gatgcgtcaatccatgacactgttgagaatctcattattctcgcgaacaac |
| | | tcactcagctcaaatgggaacgtcactgaatccggctgcaaggaatgcgag |
| | | gaattggaagagaagaatattaaagagttcctgcagagtttcgttcatatt |
| | | gttcaaatgtttatcaacacaagtggaggggggtcagggggtggctcaggc |
| | | ggcggcagtgggggcagcataacttgtccacctccgatgagtgtggaacat |
| | | gctgacatttgggtaaagagttactcactctattcacgagagcgatacatc |
| | | tgtaatagtgggtttaaaagaaaagccggtacatcctctcttactgaatgc |
| | | gtactcaataaggcgaccaacgtcgcgcactggaccactccttccttgaaa |
| | | tgcatccgcgacccggctctcgtttagcatcgatgatatcgcggccgcata |
| | | cagcagcaattggcaagctgcttacatagaactcgcggcgattggcatgcc |
| | | gccttaaaatttttattttatttttcttttcttttccgaatcggattttgt |
| | | ttttaatatttcaaaaaaaaaaaaaaaaaaaaaaaaaa |
| First nucleic | 77 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| human IL15Ra | | ggacccatccgacacgatccttgacattggaagtgcgcccgcccgcagaat |
| (Sam002- | | gtattctaagcacaagtatcattgtatctgtccgatgagatgtgcggaaga |
| hIL15Ra) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgtaccgcatgcaacttttg |
| | | tcttgcattgcacttagcttggcactcgtcactaattccattacatgccct |
| | | ccgcctatgtctgtggagcacgcagacatctgggtaaagtcttactccctc |
| | | tactcaagagaacgatatatctgtaattcagggtttaagcgaaaagcagga |
| | | acctcttcactgacagaatgcgtgcttaataaggcgacgaacgttgctcat |
| | | tggactacacctagtttgaagtgtattcgggatccggcactcgtaggaggg |
| | | gggtcagggggtggctcaggcggcggcagtgggggcagcaattgggtaaac |
| | | gtaattagcgatctgaagaagatcgaggacttgatacaaagtatgcatatc |
| | | gatgctactctttataccgagtctgatgtacacccctcttgtaaagtaaca |
| | | gcgatgaagtgctttttgttggagttgcaagtgataagcctggaaagtggg |
| | | gatgcgtccatccacgataccgtcgagaacttgataatactggccaacaac |
| | | agtctctctagtaacggtaacgtaaccgagagcgggtgtaaagaatgcgaa |
| | | gaattggaagagaaaaatatcaaagaattccttcagtcatttgtgcatata |
| | | gtgcagatgtttatcaacacatcctagcatcgatgatatcgcggccgcata |
| | | cagcagcaattggcaagctgcttacatagaactcgcggcgattggcatgcc |
| | | gccttaaaatttttattttatttttcttttcttttccgaatcggattttgt |
| | | ttttaatatttcaaaaaaaaaaaaaaaaaaaaaaaaaa |
| First nucleic | 78 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| mouse IL15Ra | | ggacccatccgacacgatccttgacattggaagtgcgcccgcccgcagaat |
| (Sam002- | | gtattctaagcacaagtatcattgtatctgtccgatgagatgtgcggaaga |
| mIL15Ra) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgtattcaatgcagcttgcc |
| | | agttgtgtcacactcactcttgtcctgctggttaattccaattggatcgat |
| | | gtccggtatgatttggagaagatcgaatctctcatccaaagcatacacatt |
| | | gacaccactttgtatactgatagcgattttcacccatcttgtaaggtgacc |
| | | gccatgaattgctttctgttggaacttcaagttatacttcacgagtattca |
| | | aacatgacactgaatgaaactgtccgaaacgtactctaccttgctaactcc |
| | | accctttcatctaataagaacgtagctgagagcggatgtaaggagtgtgag |
| | | gagctcgaagagaagacatttactgaatttcttcagagttttatccgcata |
| | | gtgcaaatgtttattaacacctccggaggggggtcagggggtggctcaggc |
| | | ggcggcagtgggggcagcggaactacatgtccacctcccgtcagtatcgaa |
| | | catgcagatatcagagttaaaaattattcagtgaacagcagggagcgctac |
| | | gtgtgtaatagtggttttaaaaggaaagccggcacttctacattgattgaa |
| | | tgcgttatcaataagaatactaacgtagctcactggaccacaccctctctg |
| | | aagtgtatccgagacccttcacttgcctagcatcgatgatatcgcggccgc |
| | | atacagcagcaattggcaagctgcttacatagaactcgcggcgattggcat |
| | | gccgccttaaaatttttattttatttttcttttcttttccgaatcggattt |
| | | tgtttttaatatttcaaaaaaaaaaaaaaaaaaaaaaaaaa |
| First nucleic | 79 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| mouse IL18 | | ggacccatccgacacgatccttgacattggaagtgcgcccgcccgcagaat |
| (Sam002- | | gtattctaagcacaagtatcattgtatctgtccgatgagatgtgcggaaga |
| IL18) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgacctcccggcttgtgagg |
| | | gtactggctgctgctatgctggtggctgctgctgtgagtgtggcaaacttt |
| | | gggagactccactgtaccactgccgttatacggaatataaacgatcaggtc |
| | | ctctttgtagacaagagacagccagttttcgaggacatgactgatatagat |
| | | cagagtgcctctgagcctcaaactcggctcataatatacatgtataaggat |
| | | agcgaggttcggggacttgccgtgactttgtccgtgaaagactcaaaaatg |
| | | agcaccctgtcatgcaaaaataagataatttctttcgaggaaatggaccct |
| | | cccgagaacattgatgatatccagtccgacttgatatttttccagaaacgg |
| | | gttcccggtcataacaaaatggagtttgagtcttcattgtacgagggtcat |
| | | tttttggcatgccagaaggaggatgatgccttcaaactgattttgaagaaa |
| | | aaagacgagaacggagacaagtctgtcatgtttacacttaccaaccttcat |
| | | caatcctagcatcgatgatatcgcggccgcatacagcagcaattggcaagc |
| | | tgcttacatagaactcgcggcgattggcatgccgccttaaaatttttattt |
| | | tatttttcttttcttttccgaatcggattttgtttttaatatttcaaaaaa |
| | | aaaaaaaaaaaaaaaaaaaa |
| First nucleic | 80 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| mutant mouse | | ggacccatccgacacgatccttgacattggaagtgcgcccgcccgcagaat |
| IL18 (Sam002- | | gtattctaagcacaagtatcattgtatctgtccgatgagatgtgcggaaga |
| IL18 mutant) | | tccggacagattgtataagtatgcaactaagctgaagaaaaactgtaagga |
| | | aataactgataaggaattggacaagaaaatgaaggagctggccgccgtcat |
| | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgacctcccggcttgtgagg |
| | | gtactggctgctgctatgctggtggctgctgctgtgagtgtggcacacttc |
| | | ggacgactccattgtactacagccgttatacgaaatatcaatgatcaggtg |
| | | ttgtttgtagacaaaaggcagcccgtgtttgaagacatgactgatattgat |
| | | cagagtgcctctgagccccagactagactgattatatatgcttatggcgac |
| | | tctagagctagagggaaagcagtaactctgtcagtgaaagacagcaagatg |
| | | tctaccctgtcttgcaagaataaaataatctcatttgaggagatggacccc |
| | | cctgaaaacatagatgacatccaaagtgacctgatattctttcagaagcgg |
| | | gtacccggtcataacaaaatggagttcgagtcaagcctctatgagggtcac |
| | | tttctcgcctgccaaaaggaagatgacgccttcaaattgatcttgaaaaaa |
| | | aaagacgagaatggtgataagtctgtcatgttcacactcactaatttgcat |
| | | cagtcatagcatcgatgatatcgcggccgcatacagcagcaattggcaagc |
| | | tgcttacatagaactcgcggcgattggcatgccgccttaaaatttttattt |
| | | tatttttcttttcttttccgaatcggattttgtttttaatatttcaaaaaa |
| | | aaaaaaaaaaaaaaaaaaaa |
| First nucleic | 81 | gcggcgcatgagagaagcccagaccaattacctacccaaaatggagaaagt |
| acid construct, | | tcacgttgacatcgaggaagacagcccattcctcagagctttgcagcggag |
| sa-mRNA | | cttcccgcagtttgaggtagaagccaagcaggtcactgataatgaccatgc |
| encoding | | taatgccagagcgttttcgcatctggcttcaaaactgatcgaaacggaggt |
| mouse | | ggacccatccgacacgatccttgacattggaagtgcgcccgcccgcagaat |
| mCXCL10-9- | | gtattctaagcacaagtatcattgtatctgtccgatgagatgtgcggaaga |
| 11-4 (Sam002- | | tccggacagattgtataagtatgcaactaagctgaagaaaaactgtaagga |
| mmCXCL10- | | aataactgataaggaattggacaagaaaatgaaggagctggccgccgtcat |
| 9-11-4) | | gagcgaccctgacctggaaactgagactatgtgcctccacgacgacgagtc |
| | | gtgtcgctacgaagggcaagtcgctgtttaccaggatgtatacgcggttga |
| | | cggaccgacaagtctctatcaccaagccaataagggagttagagtcgccta |
| | | ctggataggctttgacaccaccccttttatgtttaagaacttggctggagc |
| | | atatccatcatactctaccaactgggccgacgaaaccgtgttaacggctcg |
| | | taacataggcctatgcagctctgacgttatggagcggtcacgtagagggat |
| | | gtccattcttagaaagaagtatttgaaaccatccaacaatgttctattctc |
| | | tgttggctcgaccatctaccacgagaagagggacttactgaggagctggca |
| | | cctgccgtctgtatttcacttacgtggcaagcaaaattacacatgtcggtg |
| | | tgagactatagttagttgcgacgggtacgtcgttaaaagaatagctatcag |
| | | tccaggcctgtatgggaagccttcaggctatgctgctacgatgcaccgcga |
| | | gggattcttgtgctgcaaagtgacagacacattgaacggggagagggtctc |
| | | ttttcccgtgtgcacgtatgtgccagctacattgtgtgaccaaatgactgg |
| | | catactggcaacagatgtcagtgcggacgacgcgcaaaaactgctggttgg |
| | | gctcaaccagcgtatagtcgtcaacggtcgcacccagagaaacaccaatac |
| | | catgaaaaattaccttttgcccgtagtggcccaggcatttgctaggtgggc |
| | | aaaggaatataaggaagatcaagaagatgaaaggccactaggactacgaga |
| | | tagacagttagtcatggggtgttgttgggcttttagaaggcacaagataac |
| | | atctatttataagcgcccggatacccaaaccatcatcaaagtgaacagcga |
| | | tttccactcattcgtgctgcccaggataggcagtaacacattggagatcgg |
| | | gctgagaacaagaatcaggaaaatgttagaggagcacaaggagccgtcacc |
| | | tctcattaccgccgaggacgtacaagaagctaagtgcgcagccgatgaggc |
| | | taaggaggtgcgtgaagccgaggagttgcgcgcagctctaccacctttggc |
| | | agctgatgttgaggagcccactctggaagccgatgtcgacttgatgttaca |
| | | agaggctggggccggctcagtggagacacctcgtggcttgataaaggttac |
| | | cagctacgatggcgaggacaagatcggctcttacgctgtgctttctccgca |
| | | ggctgtactcaagagtgaaaaattatcttgcatccaccctctcgctgaaca |
| | | agtcatagtgataacacactctggccgaaaagggcgttatgccgtggaacc |
| | | ataccatggtaaagtagtggtgccagagggacatgcaatacccgtccagga |
| | | ctttcaagctctgagtgaaagtgccaccattgtgtacaacgaacgtgagtt |
| | | cgtaaacaggtacctgcaccatattgccacacatggaggagcgctgaacac |
| | | tgatgaagaatattacaaaactgtcaagcccagcgagcacgacggcgaata |
| | | cctgtacgacatcgacaggaaacagtgcgtcaagaaagaactagtcactgg |
| | | gctagggctcacaggcgagctggtggatcctcccttccatgaattcgccta |
| | | cgagagtctgagaacacgaccagccgctccttaccaagtaccaaccatagg |
| | | ggtgtatggcgtgccaggatcaggcaagtctggcatcattaaaagcgcagt |
| | | caccaaaaaagatctagtggtgagcgccaagaaagaaaactgtgcagaaat |
| | | tataagggacgtcaagaaaatgaaagggctggacgtcaatgccagaactgt |
| | | ggactcagtgctcttgaatggatgcaaacaccccgtagagaccctgtatat |
| | | tgacgaagcttttgcttgtcatgcaggtactctcagagcgctcatagccat |
| | | tataagacctaaaaaggcagtgctctgcggggatcccaaacagtgcggttt |
| | | ttttaacatgatgtgcctgaaagtgcattttaaccacgagatttgcacaca |
| | | agtcttccacaaaagcatctctcgccgttgcactaaatctgtgacttcggt |
| | | cgtctcaaccttgttttacgacaaaaaaatgagaacgacgaatccgaaaga |
| | | gactaagattgtgattgacactaccggcagtaccaaacctaagcaggacga |
| | | tctcattctcacttgtttcagagggtgggtgaagcagttgcaaatagatta |
| | | caaaggcaacgaaataatgacggcagctgcctctcaagggctgacccgtaa |
| | | aggtgtgtatgccgttcggtacaaggtgaatgaaaatcctctgtacgcacc |
| | | cacctcagaacatgtgaacgtcctactgacccgcacggaggaccgcatcgt |
| | | gtggaaaacactagccggcgacccatggataaaaacactgactgccaagta |
| | | ccctgggaatttcactgccacgatagaggagtggcaagcagagcatgatgc |
| | | catcatgaggcacatcttggagagaccggaccctaccgacgtcttccagaa |
| | | taaggcaaacgtgtgttgggccaaggctttagtgccggtgctgaagaccgc |
| | | tggcatagacatgaccactgaacaatggaacactgtggattattttgaaac |
| | | ggacaaagctcactcagcagagatagtattgaaccaactatgcgtgaggtt |
| | | ctttggactcgatctggactccggtctattttctgcacccactgttccgtt |
| | | atccattaggaataatcactgggataactccccgtcgcctaacatgtacgg |
| | | gctgaataaagaagtggtccgtcagctctctcgcaggtacccacaactgcc |
| | | tcgggcagttgccactggaagagtctatgacatgaacactggtacactgcg |
| | | caattatgatccgcgcataaacctagtacctgtaaacagaagactgcctca |
| | | tgctttagtcctccaccataatgaacacccacagagtgacttttcttcatt |
| | | cgtcagcaaattgaagggcagaactgtcctggtggtcggggaaaagttgtc |
| | | cgtcccaggcaaaatggttgactggttgtcagaccggcctgaggctacctt |
| | | cagagctcggctggatttaggcatcccaggtgatgtgcccaaatatgacat |
| | | aatatttgttaatgtgaggaccccatataaataccatcactatcagcagtg |
| | | tgaagaccatgccattaagcttagcatgttgaccaagaaagcttgtctgca |
| | | tctgaatcccggcggaacctgtgtcagcataggttatggttacgctgacag |
| | | ggccagcgaaagcatcattggtgctatagcgcggcagttcaagttttcccg |
| | | ggtatgcaaaccgaaatcctcacttgaagagacggaagttctgtttgtatt |
| | | cattgggtacgatcgcaaggcccgtacgcacaattcttacaagctttcatc |
| | | aaccttgaccaacatttatacaggttccagactccacgaagccggatgtgc |
| | | accctcatatcatgtggtgcgaggggatattgccacggccaccgaaggagt |
| | | gattataaatgctgctaacagcaaaggacaacctggcggaggggtgtgcgg |
| | | agcgctgtataagaaattcccggaaagcttcgatttacagccgatcgaagt |
| | | aggaaaagcgcgactggtcaaaggtgcagctaaacatatcattcatgccgt |
| | | aggaccaaacttcaacaaagtttcggaggttgaaggtgacaaacagttggc |
| | | agaggcttatgagtccatcgctaagattgtcaacgataacaattacaagtc |
| | | agtagcgattccactgttgtccaccggcatcttttccgggaacaaagatcg |
| | | actaacccaatcattgaaccatttgctgacagctttagacaccactgatgc |
| | | agatgtagccatatactgcagggacaagaaatgggaaatgactctcaagga |
| | | agcagtggctaggagagaagcagtggaggagatatgcatatccgacgactc |
| | | ttcagtgacagaacctgatgcagagctggtgagggtgcatccgaagagttc |
| | | tttggctggaaggaagggctacagcacaagcgatggcaaaactttctcata |
| | | tttggaagggaccaagtttcaccaggcggccaaggatatagcagaaattaa |
| | | tgccatgtggcccgttgcaacggaggccaatgagcaggtatgcatgtatat |
| | | cctcggagaaagcatgagcagtattaggtcgaaatgccccgtcgaagagtc |
| | | ggaagcctccacaccacctagcacgctgccttgcttgtgcatccatgccat |
| | | gactccagaaagagtacagcgcctaaaagcctcacgtccagaacaaattac |
| | | tgtgtgctcatcctttccattgccgaagtatagaatcactggtgtgcagaa |
| | | gatccaatgctcccagcctatattgttctcaccgaaagtgcctgcgtatat |
| | | tcatccaaggaagtatctcgtggaaacaccaccggtagacgagactccgga |
| | | gccatcggcagagaaccaatccacagaggggacacctgaacaaccaccact |
| | | tataaccgaggatgagaccaggactagaacgcctgagccgatcatcatcga |
| | | agaggaagaagaggatagcataagtttgctgtcagatggcccgacccacca |
| | | ggtgctgcaagtcgaggcagacattcacgggccgccctctgtatctagctc |
| | | atcctggtccattcctcatgcatccgactttgatgtggacagtttatccat |
| | | acttgacaccctggagggagctagcgtgaccagcggggcaacgtcagccga |
| | | gactaactcttacttcgcaaagagtatggagtttctggcgcgaccggtgcc |
| | | tgcgcctcgaacagtattcaggaaccctccacatcccgctccgcgcacaag |
| | | aacaccgtcacttgcacccagcagggcctgctcgagaaccagcctagtttc |
| | | caccccgccaggcgtgaatagggtgatcactagagaggagctcgaggcgct |
| | | taccccgtcacgcactcctagcaggtcggtctcgagaaccagcctggtctc |
| | | caacccgccaggcgtaaatagggtgattacaagagaggagtttgaggcgtt |
| | | cgtagcacaacaacaatgacggtttgatgcgggtgcatacatcttttcctc |
| | | cgacaccggtcaagggcatttacaacaaaaatcagtaaggcaaacggtgct |
| | | atccgaagtggtgttggagaggaccgaattggagatttcgtatgccccgcg |
| | | cctcgaccaagaaaaagaagaattactacgcaagaaattacagttaaatcc |
| | | cacacctgctaacagaagcagataccagtccaggaaggtggagaacatgaa |
| | | agccataacagctagacgtattctgcaaggcctagggcattatttgaaggc |
| | | agaaggaaaagtggagtgctaccgaaccctgcatcctgttcctttgtattc |
| | | atctagtgtgaaccgtgccttttcaagccccaaggtcgcagtggaagcctg |
| | | taacgccatgttgaaagagaactttccgactgtggcttcttactgtattat |
| | | tccagagtacgatgcctatttggacatggttgacggagcttcatgctgctt |
| | | agacactgccagtttttgccctgcaaagctgcgcagctttccaaagaaaca |
| | | ctcctatttggaacccacaatacgatcggcagtgccttcagcgatccagaa |
| | | cacgctccagaacgtcctggcagctgccacaaaaagaaattgcaatgtcac |
| | | gcaaatgagagaattgcccgtattggattcggcggcctttaatgtggaatg |
| | | cttcaagaaatatgcgtgtaataatgaatattgggaaacgtttaaagaaaa |
| | | ccccatcaggcttactgaagaaaacgtggtaaattacattaccaaattaaa |
| | | aggaccaaaagctgctgctctttttgcgaagacacataatttgaatatgtt |
| | | gcaggacataccaatggacaggtttgtaatggacttaaagagagacgtgaa |
| | | agtgactccaggaacaaaacatactgaagaacggcccaaggtacaggtgat |
| | | ccaggctgccgatccgctagcaacagcgtatctgtgcggaatccaccgaga |
| | | gctggttaggagattaaatgcggtcctgcttccgaacattcatacactgtt |
| | | tgatatgtcggctgaagactttgacgctattatagccgagcacttccagcc |
| | | tggggattgtgttctggaaactgacatcgcgtcgtttgataaaagtgagga |
| | | cgacgccatggctctgaccgcgttaatgattctggaagacttaggtgtgga |
| | | cgcagagctgttgacgctgattgaggcggctttcggcgaaatttcatcaat |
| | | acatttgcccactaaaactaaatttaaattcggagccatgatgaaatctgg |
| | | aatgttcctcacactgtttgtgaacacagtcattaacattgtaatcgcaag |
| | | cagagtgttgagagaacggctaaccggatcaccatgtgcagcattcattgg |
| | | agatgacaatatcgtgaaaggagtcaaatcggacaaattaatggcagacag |
| | | gtgcgccacctggttgaatatggaagtcaagattatagatgctgtggtggg |
| | | cgagaaagcgccttatttctgtggagggtttattttgtgtgactccgtgac |
| | | cggcacagcgtgccgtgtggcagaccccctaaaaaggctgtttaagcttgg |
| | | caaacctctggcagcagacgatgaacatgatgatgacaggagaagggcatt |
| | | gcatgaagagtcaacacgctggaaccgagtgggtattctttcagagctgtg |
| | | caaggcagtagaatcaaggtatgaaaccgtaggaacttccatcatagttat |
| | | ggccatgactactctagctagcagtgttaaatcattcagctacctgagagg |
| | | ggcccctataactctctacggctaacctgaatgaatccatccgcagctgta |
| | | attttctgccttatcctcctggggctgagtggaacccaaggaatccccctc |
| | | gctcgaacagtacgctgcaactgtatccacatagacgatggccccgttcga |
| | | atgagggctatcggaaagctcgaaataatacccgcctctctctcatgcccc |
| | | cgcgtagaaataatagcaactatgaagaagaacgacgagcaacgatgcttg |
| | | aacccagaaagcaagactatcaagaacttgatgaaagcttttagccaaaag |
| | | cggagtaaacgagcccccgggggaggcggctccggcggaggagggagtggt |
| | | ggcggtggatccggaggcggtgggagtacccttgtgattaggaatgcacga |
| | | tgcagctgtataagcacctcccggggcactattcactataagagccttaaa |
| | | gatctcaagcagtttgcaccatctccaaattgtaacaaaactgagattatt |
| | | gctacactgaagaacggggaccaaacctgcctggacccagatagtgccaat |
| | | gttaaaaagcttatgaaggaatgggagaaaaaaatcagtcaaaagaaaaag |
| | | caaaaacgcggtaaaaaacaccagaaaaacatgaagaatcggaaaccaaaa |
| | | accccacagtctaggagaaggagtcgaaaaacaacagggggaggcggaagt |
| | | ggcggcgggggcagcggtggcgggggatcaggcggcgggggcagtttcctt |
| | | atgttcaaacaagggcggtgcctgtgtatagggccaggtatgaaagcagtg |
| | | aagatggctgaaatcgaaaaagcaagtgtgatctaccctagtaatggctgt |
| | | gataaagttgaagttattgtgactatgaaagctcacaagagacagcggtgt |
| | | ttggatcctagatccaaacaagcccgactcatcatgcaagccatagaaaag |
| | | aagaacttcctgcgaaggcagaatatgggtggaggtggaagcggtggcggg |
| | | ggtagcggcgggggaggtagtggtgggggcggtagcgtaactagtgctgga |
| | | ccagaggagtccgatggggatctctcttgtgtctgcgttaagaccatatca |
| | | tccgggattcaccttaagcacattaccagcctcgaagtgatcaaagctggt |
| | | aggcattgtgccgtgccccagctcatagccacattgaagaatgggaggaaa |
| | | atttgccttgatagacaggcaccattgtataagaaagtcattaaaaaaatt |
| | | cttgaatcatagcatcgatgatatcgcggccgcatacagcagcaattggca |
| | | agctgcttacatagaactcgcggcgattggcatgccgccttaaaattttta |
| | | ttttatttttcttttcttttccgaatcggattttgtttttaatatttcaaa |
| | | aaaaaaaaaaaaaaaaaaaaaaa |
| Nucleic Acid | 84 | aaaaaaaaaacgcgtgataacgcaggaaagaacatgtgagcaaaaggccag |
| Template, sa- | | caaaaggccaggaaccgtaaaaaggccgcgttgctggcgtttttccatagg |
| mRNA | | ctccgcccccctgacgagcatcacaaaaatcgacgctcaagtcagaggtgg |
| encoding GFP | | cgaaacccgacaggactataaagataccaggcgtttccccctggaagctcc |
| (SVP-018)- | | ctcgtgcgctctcctgttccgaccctgccgcttaccggatacctgtccgcc |
| the selectable | | tttctcccttcgggaagcgtggcgctttctcatagctcacgctgtaggtat |
| marker (Amp | | ctcagttcggtgtaggtcgttcgctccaagctgggctgtgtgcacgaaccc |
| Cassette, SEQ | | cccgttcagcccgaccgctgcgccttatccggtaactatcgtcttgagtcc |
| ID NO: 12) is | | aacccggtaagacacgacttatcgccactggcagcagccactggtaacagg |
| bolded and | | attagcagagcgaggtatgtaggcggtgctacagagttcttgaagtggtgg |
| italicized; the | | cctaactacggctacactagaagaacagtatttggtatctgcgctctgctg |
| coding | | aagccagttaccttcggaaaaagagttggtagctcttgatccggcaaacaa |
| sequences of | | accaccgctggtagcggtttttttgtttgcaagcagcagattacgcgcaga |
| structural | | aaaaaaggatctcaagaagatcctttgatcttttctacggggtctgacgct |
| proteins of of | | cagtggaacgaaaactcacgttaagggattttggtcatgagattatcaaaa |
| SFV4 are | | aggatcttcacctagatccttttaaattaaaaatgaagttttaaatcaatc |
| italicized and | | taaagtatatatgagtaaacttggtctgacagttaccaatgcttaatcagt |
| underlined; | | gaggcacctatctcagcgatctgtctatttcgttcatccatagttgcctga |
| and viral | | ctccccgtcgtgtagataactacgatacgggagggcttaccatctggcccc |
| structural | | agtgctgcaatgataccgcgagacccacgctcaccggctccagatttatca |
| proteins from | | gcaataaaccagccagccggaagggccgagcgcagaagtggtcctgcaact |
| SFV4 is | | ttatccgcctccatccagtctattaattgttgccgggaagctagagtaagt |
| bolded. The | | agttcgccagttaatagtttgcgcaacgttgttgccattgctacaggcatc |
| selectable | | gtggtgtcacgctcgtcgtttggtatggcttcattcagctccggttcccaa |
| marker and | | cgatcaaggcgagttacatgatcccccatgttgtgcaaaaaagcggttagc |
| GOI can be | | tccttcggtcctccgatcgttgtcagaagtaagttggccgcagtgttatca |
| substituted | | ctcatggttatggcagcactgcataattctcttactgtcatgccatccgta |
| with one or | | agatgcttttctgtgactggtgagtactcaaccaagtcattctgagaatag |
| more different | | tgtatgcggcgaccgagttgctcttgcccggcgtcaatacgggataatacc |
| selectable | | gcgccacatagcagaactttaaaagtgctcatcattggaaaacgttcttcg |
| markers and | | gggcgaaaactctcaaggatcttaccgctgttgagatccagttcgatgtaa |
| GOIs. | | cccactcgtgcacccaactgatcttcagcatcttttactttcaccagcgtt |
| | | tctgggtgagcaaaaacaggaaggcaaaatgccgcaaaaaagggaataagg |
| | | gcgacacggaaatgttgaatactcatactcttcctttttcaatattattga |
| | | agcatttatcagggttattgtctcatgagcggatacatatttgaatgtatt |
| | | tagaaaaataaacaaataggggttccgcgcacatttccccgaaaagtgcca |
| | | cctgagctctaatacgactcactataggataggcggcgcatgagagaagcc |
| | | cagaccaattacctacccaaaatggagaaagttcacgttgacatcgaggaa |
| | | gacagcccattcctcagagctttgcagcggagcttcccgcagtttgaggta |
| | | gaagccaagcaggtcactgataatgaccatgctaatgccagagcgttttcg |
| | | catctggcttcaaaactgatcgaaacggaggtggacccatccgacacgatc |
| | | cttgacattggaagtgcgcccgcccgcagaatgtattctaagcacaagtat |
| | | cattgtatctgtccgatgagatgtgcggaagatccggacagattgtataag |
| | | tatgcaactaagctgaagaaaaactgtaaggaaataactgataaggaattg |
| | | gacaagaaaatgaaggagctggccgccgtcatgagcgaccctgacctggaa |
| | | actgagactatgtgcctccacgacgacgagtcgtgtcgctacgaagggcaa |
| | | gtcgctgtttaccaggatgtatacgcggttgacggaccgacaagtctctat |
| | | caccaagccaataagggagttagagtcgcctactggataggctttgacacc |
| | | accccttttatgtttaagaacttggctggagcatatccatcatactctacc |
| | | aactgggccgacgaaaccgtgttaacggctcgtaacataggcctatgcagc |
| | | tctgacgttatggagcggtcacgtagagggatgtccattcttagaaagaag |
| | | tatttgaaaccatccaacaatgttctattctctgttggctcgaccatctac |
| | | cacgagaagagggacttactgaggagctggcacctgccgtctgtatttcac |
| | | ttacgtggcaagcaaaattacacatgtcggtgtgagactatagttagttgc |
| | | gacgggtacgtcgttaaaagaatagctatcagtccaggcctgtatgggaag |
| | | ccttcaggctatgctgctacgatgcaccgcgagggattcttgtgctgcaaa |
| | | gtgacagacacattgaacggggagagggtctcttttcccgtgtgcacgtat |
| | | gtgccagctacattgtgtgaccaaatgactggcatactggcaacagatgtc |
| | | agtgcggacgacgcgcaaaaactgctggttgggctcaaccagcgtatagtc |
| | | gtcaacggtcgcacccagagaaacaccaataccatgaaaaattaccttttg |
| | | cccgtagtggcccaggcatttgctaggtgggcaaaggaatataaggaagat |
| | | caagaagatgaaaggccactaggactacgagatagacagttagtcatgggg |
| | | tgttgttgggcttttagaaggcacaagataacatctatttataagcgcccg |
| | | gatacccaaaccatcatcaaagtgaacagcgatttccactcattcgtgctg |
| | | cccaggataggcagtaacacattggagatcgggctgagaacaagaatcagg |
| | | aaaatgttagaggagcacaaggagccgtcacctctcattaccgccgaggac |
| | | gtacaagaagctaagtgcgcagccgatgaggctaaggaggtgcgtgaagcc |
| | | gaggagttgcgcgcagctctaccacctttggcagctgatgttgaggagccc |
| | | actctggaagccgatgtcgacttgatgttacaagaggctggggccggctca |
| | | gtggagacacctcgtggcttgataaaggttaccagctacgatggcgaggac |
| | | aagatcggctcttacgctgtgctttctccgcaggctgtactcaagagtgaa |
| | | aaattatcttgcatccaccctctcgctgaacaagtcatagtgataacacac |
| | | tctggccgaaaagggcgttatgccgtggaaccataccatggtaaagtagtg |
| | | gtgccagagggacatgcaatacccgtccaggactttcaagctctgagtgaa |
| | | agtgccaccattgtgtacaacgaacgtgagttcgtaaacaggtacctgcac |
| | | catattgccacacatggaggagcgctgaacactgatgaagaatattacaaa |
| | | actgtcaagcccagcgagcacgacggcgaatacctgtacgacatcgacagg |
| | | aaacagtgcgtcaagaaagaactagtcactgggctagggctcacaggcgag |
| | | ctggtggatcctcccttccatgaattcgcctacgagagtctgagaacacga |
| | | ccagccgctccttaccaagtaccaaccataggggtgtatggcgtgccagga |
| | | tcaggcaagtctggcatcattaaaagcgcagtcaccaaaaaagatctagtg |
| | | gtgagcgccaagaaagaaaactgtgcagaaattataagggacgtcaagaaa |
| | | atgaaagggctggacgtcaatgccagaactgtggactcagtgctcttgaat |
| | | ggatgcaaacaccccgtagagaccctgtatattgacgaagcttttgcttgt |
| | | catgcaggtactctcagagcgctcatagccattataagacctaaaaaggca |
| | | gtgctctgcggggatcccaaacagtgcggtttttttaacatgatgtgcctg |
| | | aaagtgcattttaaccacgagatttgcacacaagtcttccacaaaagcatc |
| | | tctcgccgttgcactaaatctgtgacttcggtcgtctcaaccttgttttac |
| | | gacaaaaaaatgagaacgacgaatccgaaagagactaagattgtgattgac |
| | | actaccggcagtaccaaacctaagcaggacgatctcattctcacttgtttc |
| | | agagggtgggtgaagcagttgcaaatagattacaaaggcaacgaaataatg |
| | | acggcagctgcctctcaagggctgacccgtaaaggtgtgtatgccgttcgg |
| | | tacaaggtgaatgaaaatcctctgtacgcacccacctcagaacatgtgaac |
| | | gtcctactgacccgcacggaggaccgcatcgtgtggaaaacactagccggc |
| | | gacccatggataaaaacactgactgccaagtaccctgggaatttcactgcc |
| | | acgatagaggagtggcaagcagagcatgatgccatcatgaggcacatcttg |
| | | gagagaccggaccctaccgacgtcttccagaataaggcaaacgtgtgttgg |
| | | gccaaggctttagtgccggtgctgaagaccgctggcatagacatgaccact |
| | | gaacaatggaacactgtggattattttgaaacggacaaagctcactcagca |
| | | gagatagtattgaaccaactatgcgtgaggttctttggactcgatctggac |
| | | tccggtctattttctgcacccactgttccgttatccattaggaataatcac |
| | | tgggataactccccgtcgcctaacatgtacgggctgaataaagaagtggtc |
| | | cgtcagctctctcgcaggtacccacaactgcctcgggcagttgccactgga |
| | | agagtctatgacatgaacactggtacactgcgcaattatgatccgcgcata |
| | | aacctagtacctgtaaacagaagactgcctcatgctttagtcctccaccat |
| | | aatgaacacccacagagtgacttttcttcattcgtcagcaaattgaagggc |
| | | agaactgtcctggtggtcggggaaaagttgtccgtcccaggcaaaatggtt |
| | | gactggttgtcagaccggcctgaggctaccttcagagctcggctggattta |
| | | ggcatcccaggtgatgtgcccaaatatgacataatatttgttaatgtgagg |
| | | accccatataaataccatcactatcagcagtgtgaagaccatgccattaag |
| | | cttagcatgttgaccaagaaagcttgtctgcatctgaatcccggcggaacc |
| | | tgtgtcagcataggttatggttacgctgacagggccagcgaaagcatcatt |
| | | ggtgctatagcgcggcagttcaagttttcccgggtatgcaaaccgaaatcc |
| | | tcacttgaagagacggaagttctgtttgtattcattgggtacgatcgcaag |
| | | gcccgtacgcacaattcttacaagctttcatcaaccttgaccaacatttat |
| | | acaggttccagactccacgaagccggatgtgcaccctcatatcatgtggtg |
| | | cgaggggatattgccacggccaccgaaggagtgattataaatgctgctaac |
| | | agcaaaggacaacctggcggaggggtgtgcggagcgctgtataagaaattc |
| | | ccggaaagcttcgatttacagccgatcgaagtaggaaaagcgcgactggtc |
| | | aaaggtgcagctaaacatatcattcatgccgtaggaccaaacttcaacaaa |
| | | gtttcggaggttgaaggtgacaaacagttggcagaggcttatgagtccatc |
| | | gctaagattgtcaacgataacaattacaagtcagtagcgattccactgttg |
| | | tccaccggcatcttttccgggaacaaagatcgactaacccaatcattgaac |
| | | catttgctgacagctttagacaccactgatgcagatgtagccatatactgc |
| | | agggacaagaaatgggaaatgactctcaaggaagcagtggctaggagagaa |
| | | gcagtggaggagatatgcatatccgacgactcttcagtgacagaacctgat |
| | | gcagagctggtgagggtgcatccgaagagttctttggctggaaggaagggc |
| | | tacagcacaagcgatggcaaaactttctcatatttggaagggaccaagttt |
| | | caccaggcggccaaggatatagcagaaattaatgccatgtggcccgttgca |
| | | acggaggccaatgagcaggtatgcatgtatatcctcggagaaagcatgagc |
| | | agtattaggtcgaaatgccccgtcgaagagtcggaagcctccacaccacct |
| | | agcacgctgccttgcttgtgcatccatgccatgactccagaaagagtacag |
| | | cgcctaaaagcctcacgtccagaacaaattactgtgtgctcatcctttcca |
| | | ttgccgaagtatagaatcactggtgtgcagaagatccaatgctcccagcct |
| | | atattgttctcaccgaaagtgcctgcgtatattcatccaaggaagtatctc |
| | | gtggaaacaccaccggtagacgagactccggagccatcggcagagaaccaa |
| | | tccacagaggggacacctgaacaaccaccacttataaccgaggatgagacc |
| | | aggactagaacgcctgagccgatcatcatcgaagaggaagaagaggatagc |
| | | ataagtttgctgtcagatggcccgacccaccaggtgctgcaagtcgaggca |
| | | gacattcacgggccgccctctgtatctagctcatcctggtccattcctcat |
| | | gcatccgactttgatgtggacagtttatccatacttgacaccctggaggga |
| | | gctagcgtgaccagcggggcaacgtcagccgagactaactcttacttcgca |
| | | aagagtatggagtttctggcgcgaccggtgcctgcgcctcgaacagtattc |
| | | aggaaccctccacatcccgctccgcgcacaagaacaccgtcacttgcaccc |
| | | agcagggcctgctcgagaaccagcctagtttccaccccgccaggcgtgaat |
| | | agggtgatcactagagaggagctcgaggcgcttaccccgtcacgcactcct |
| | | agcaggtcggtctcgagaaccagcctggtctccaacccgccaggcgtaaat |
| | | agggtgattacaagagaggagtttgaggcgttcgtagcacaacaacaatga |
| | | cggtttgatgcgggtgcatacatcttttcctccgacaccggtcaagggcat |
| | | ttacaacaaaaatcagtaaggcaaacggtgctatccgaagtggtgttggag |
| | | aggaccgaattggagatttcgtatgccccgcgcctcgaccaagaaaaagaa |
| | | gaattactacgcaagaaattacagttaaatcccacacctgctaacagaagc |
| | | agataccagtccaggaaggtggagaacatgaaagccataacagctagacgt |
| | | attctgcaaggcctagggcattatttgaaggcagaaggaaaagtggagtgc |
| | | taccgaaccctgcatcctgttcctttgtattcatctagtgtgaaccgtgcc |
| | | ttttcaagccccaaggtcgcagtggaagcctgtaacgccatgttgaaagag |
| | | aactttccgactgtggcttcttactgtattattccagagtacgatgcctat |
| | | ttggacatggttgacggagcttcatgctgcttagacactgccagtttttgc |
| | | cctgcaaagctgcgcagctttccaaagaaacactcctatttggaacccaca |
| | | atacgatcggcagtgccttcagcgatccagaacacgctccagaacgtcctg |
| | | gcagctgccacaaaaagaaattgcaatgtcacgcaaatgagagaattgccc |
| | | gtattggattcggcggcctttaatgtggaatgcttcaagaaatatgcgtgt |
| | | aataatgaatattgggaaacgtttaaagaaaaccccatcaggcttactgaa |
| | | gaaaacgtggtaaattacattaccaaattaaaaggaccaaaagctgctgct |
| | | ctttttgcgaagacacataatttgaatatgttgcaggacataccaatggac |
| | | aggtttgtaatggacttaaagagagacgtgaaagtgactccaggaacaaaa |
| | | catactgaagaacggcccaaggtacaggtgatccaggctgccgatccgcta |
| | | gcaacagcgtatctgtgcggaatccaccgagagctggttaggagattaaat |
| | | gcggtcctgcttccgaacattcatacactgtttgatatgtcggctgaagac |
| | | tttgacgctattatagccgagcacttccagcctggggattgtgttctggaa |
| | | actgacatcgcgtcgtttgataaaagtgaggacgacgccatggctctgacc |
| | | gcgttaatgattctggaagacttaggtgtggacgcagagctgttgacgctg |
| | | attgaggcggctttcggcgaaatttcatcaatacatttgcccactaaaact |
| | | aaatttaaattcggagccatgatgaaatctggaatgttcctcacactgttt |
| | | gtgaacacagtcattaacattgtaatcgcaagcagagtgttgagagaacgg |
| | | ctaaccggatcaccatgtgcagcattcattggagatgacaatatcgtgaaa |
| | | ggagtcaaatcggacaaattaatggcagacaggtgcgccacctggttgaat |
| | | atggaagtcaagattatagatgctgtggtgggcgagaaagcgccttatttc |
| | | tgtggagggtttattttgtgtgactccgtgaccggcacagcgtgccgtgtg |
| | | gcagaccccctaaaaaggctgtttaagcttggcaaacctctggcagcagac |
| | | gatgaacatgatgatgacaggagaagggcattgcatgaagagtcaacacgc |
| | | tggaaccgagtgggtattctttcagagctgtgcaaggcagtagaatcaagg |
| | | tatgaaaccgtaggaacttccatcatagttatggccatgactactctagct |
| | | agcagtgttaaatcattcagctacctgagaggggcccctataactctctac |
| | | ggctaacctgaatggactacgacatagtctagtccgccaagttcgaaggcg |
| | | cgcctctagagccaccatggtgagcaagggcgaggagctgttcaccggggt |
| | | ggtgcccatcctggtcgagctggacggcgacgtaaacggccacaagttcag |
| | | cgtgtccggcgagggcgagggcgatgccacctacggcaagctgaccctgaa |
| | | gttcatctgcaccaccggcaagctgcccgtgccctggcccaccctcgtgac |
| | | caccctgacctacggcgtgcagtgcttcagccgctaccccgaccacatgaa |
| | | gcagcacgacttcttcaagtccgccatgcccgaaggctacgtccaggagcg |
| | | caccatcttcttcaaggacgacggcaactacaagacccgcgccgaggtgaa |
| | | gttcgagggcgacaccctggtgaaccgcatcgagctgaagggcatcgactt |
| | | caaggaggacggcaacatcctggggcacaagctggagtacaactacaacag |
| | | ccacaacgtctatatcatggccgacaagcagaagaacggcatcaaggtgaa |
| | | cttcaagatccgccacaacatcgaggacggcagcgtgcagctcgccgacca |
| | | ctaccagcagaacacccccatcggcgacggccccgtgctgctgcccgacaa |
| | | ccactacctgagcacccagtccgccctgagcaaagaccccaacgagaagcg |
| | | cgatcacatggtcctgctggagttcgtgaccgccgccgggatcactctcgg |
| | | catggacgagctgtacaagtagcatcgatgatatcgcggccgcatacagca |
| | | gcaattggcaagctgcttacatagaactcgcggcgattggcatgccgcctt |
| | | aaaatttttattttatttttcttttcttttccgaatcggattttgttttta |
| | | atatttcaaaaaaaaaaaaaaaa |
| Nucleic Acid | 85 | agccacaccctggagctagcaaaaaaaaaaaaaaaaaaaaaaaaaaaaaag |
| Template, | | catatgactaaaaaaaaaaaaaaaaaaaaaaaaaaaaaaaaaaaaaaaaaa |
| modified | | aaaaaaaaaaaaaaaaaaaaaaaaaaaacgcgtatcgatgatatcgcggcc |
| mRNA | | gcatacagcagcaattggcaagctgcttacatagaactcgcggcgattggc |
| encoding viral | | atgccgccttaaaatttttattttatttttcttttcttttccgaatcggat |
| structural | | tttgtttttaatatttcaaaaaaaaaaaaaaaaaaaaaaaaaacgcgtgat |
| proteins from | | aacgcaggaaagaacatgtgagcaaaaggccagcaaaaggccaggaaccgt |
| SFV4 (SVP- | | aaaaaggccgcgttgctggcgtttttccataggctccgcccccctgacgag |
| 431) -the | | catcacaaaaatcgacgctcaagtcagaggtggcgaaacccgacaggacta |
| selectable | | taaagataccaggcgtttccccctggaagctccctcgtgcgctctcctgtt |
| marker (Amp | | ccgaccctgccgcttaccggatacctgtccgcctttctcccttcgggaagc |
| Cassette, SEQ | | gtggcgctttctcatagctcacgctgtaggtatctcagttcggtgtaggtc |
| ID NO: 12) is | | gttcgctccaagctgggctgtgtgcacgaaccccccgttcagcccgaccgc |
| bolded and | | tgcgccttatccggtaactatcgtcttgagtccaacccggtaagacacgac |
| italicized and | | ttatcgccactggcagcagccactggtaacaggattagcagagcgaggtat |
| the GOI (GFP, | | gtaggcggtgctacagagttcttgaagtggtggcctaactacggctacact |
| SEQ ID NO: | | agaagaacagtatttggtatctgcgctctgctgaagccagttaccttcgga |
| 24) is bolded. | | aaaagagttggtagctcttgatccggcaaacaaaccaccgctggtagcggt |
| The selectable | | ttttttgtttgcaagcagcagattacgcgcagaaaaaaaggatctcaagaa |
| marker and | | gatcctttgatcttttctacggggtctgacgctcagtggaacgaaaactca |
| GOI can be | | cgttaagggattttggtcatgagattatcaaaaaggatcttcacctagatc |
| substituted | | cttttaaattaaaaatgaagttttaaatcaatctaaagtatatatgagtaa |
| with one or | | acttggtctgacagttaccaatgcttaatcagtgaggcacctatctcagcg |
| more different | | atctgtctatttcgttcatccatagttgcctgactccccgtcgtgtagata |
| selectable | | actacgatacgggagggcttaccatctggccccagtgctgcaatgataccg |
| markers and | | cgagacccacgctcaccggctccagatttatcagcaataaaccagccagcc |
| GOIs. | | ggaagggccgagcgcagaagtggtcctgcaactttatccgcctccatccag |
| | | tctattaattgttgccgggaagctagagtaagtagttcgccagttaatagt |
| | | ttgcgcaacgttgttgccattgctacaggcatcgtggtgtcacgctcgtcg |
| | | tttggtatggcttcattcagctccggttcccaacgatcaaggcgagttaca |
| | | tgatcccccatgttgtgcaaaaaagcggttagctccttcggtcctccgatc |
| | | gttgtcagaagtaagttggccgcagtgttatcactcatggttatggcagca |
| | | ctgcataattctcttactgtcatgccatccgtaagatgcttttctgtgact |
| | | ggtgagtactcaaccaagtcattctgagaatagtgtatgcggcgaccgagt |
| | | tgctcttgcccggcgtcaatacgggataataccgcgccacatagcagaact |
| | | ttaaaagtgctcatcattggaaaacgttcttcggggcgaaaactctcaagg |
| | | atcttaccgctgttgagatccagttcgatgtaacccactcgtgcacccaac |
| | | tgatcttcagcatcttttactttcaccagcgtttctgggtgagcaaaaaca |
| | | ggaaggcaaaatgccgcaaaaaagggaataagggcgacacggaaatgttga |
| | | atactcatactcttcctttttcaatattattgaagcatttatcagggttat |
| | | tgtctcatgagcggatacatatttgaatgtatttagaaaaataaacaaata |
| | | ggggttccgcgcacatttccccgaaaagtgccacctgagctctaatacgac |
| | | tcactataggacatttgcttctgacacaactgtgttcactagcaacctcaa |
| | | acagacaccatgaattacatccctacgcaaacgttttacgggcgcagatgg |
| | | cgacctcgacccgccgctcgcccctggcctttgcaagctacgcccgtagcc |
| | | cccgtggtcccagactttcaggcacagcaaatgcagcagcaactcatcagc |
| | | gccgtaaatgcgctgacaatgagacagaacgcaattgctcctgctaggcct |
| | | cccaaaccaaagaagaagaagacaaccaaaccaaagccgaaaacgcagccc |
| | | aagaagatcaacggaaaaacgcagcagcaaaagaagaaagacaagcaagcc |
| | | gacaagaagaagaagaaacccggaaaaagagaaagaatgtgcatgaagatt |
| | | gaaaatgactgtatcttcgaagtcaaacacgaaggaaaggtcactgggtac |
| | | gcctgcctggtgggcgacaaagtcatgaaacctgcccacgtgaaaggagtc |
| | | atcgacaacgcggacctggcaaagctagctttcaagaaatcgagcaagtat |
| | | gaccttgagtgtgcccagataccagttcacatgaggtcggatgcctcaaag |
| | | tacacgcatgagaagcccgagggacactataactggcaccacggggctgtt |
| | | cagtacagcggaggtaggttcactataccgacaggagcgggcaaaccggga |
| | | gacagtggccggcccatctttgacaacaaggggagggtagtcgctatcgtc |
| | | ctgggcggggccaacgagggctcacgcacagcactgtcggtggtcacctgg |
| | | aacaaagatatggtgactagagtgacccccgaggggtccgaagagtggtcc |
| | | gccccgctgattactgccatgtgtgtccttgccaatgctaccttcccgtgc |
| | | ttccagcccccgtgtgtaccttgctgctatgaaaacaacgcagaggccaca |
| | | ctacggatgctcgaggataacgtggataggccagggtactacgacctcctt |
| | | caggcagccttgacgtgccgaaacggaacaagacaccggcgcagcgtgtcg |
| | | caacacttcaacgtgtataaggctacacgcccttacatcgcgtactgcgcc |
| | | gactgcggagcagggcactcgtgtcatagccccgtagcaattgaagcggtc |
| | | aggtccgaagctaccgacgggatgctgaagattcagttctcggcacaaatt |
| | | ggcatagataagagtgacaatcatgactacacgaagataaggtacgcagac |
| | | gggcacgccattgagaatgccgtccggtcatctttgaaggtagccacctcc |
| | | ggagactgtttcgtccatggcacaatgggacatttcatactggcaaagtgc |
| | | ccaccgggtgaattcctgcaggtctcgatccaggacaccagaaacgcggtc |
| | | cgtgcctgcagaatacaatatcatcatgaccctcaaccggtgggtagagaa |
| | | aaatttacaattagaccacactatggaaaagagatcccttgcaccacttat |
| | | caacagaccacagcgaagaccgtggaggaaatcgacatgcatatgccgcca |
| | | gatacgccggacaggacgttgctatcacagcaatctggcaatgtaaagatc |
| | | acagtcggaggaaagaaggtgaaatacaactgcacctgtggaaccggaaac |
| | | gttggcactactaattcggacatgacgatcaacacgtgtctaatagagcag |
| | | tgccacgtctcagtgacggaccataagaaatggcagttcaactcacctttc |
| | | gtcccgagagccgacgaaccggctagaaaaggcaaagtccatatcccattc |
| | | ccgttggacaacatcacatgcagagttccaatggcgcgcgaaccaaccgtc |
| | | atccacggcaaaagagaagtgacactgcaccttcacccagatcatcccacg |
| | | ctcttttcctaccgcacactgggtgaggacccgcagtatcacgaggaatgg |
| | | gtgacagcggcggtggaacggaccatacccgtaccagtggacgggatggag |
| | | taccactggggaaacaacgacccagtgaggctttggtctcaactcaccact |
| | | gaagggaaaccgcacggctggccgcatcagatcgtacagtactactatggg |
| | | ctttacccggccgctacagtatccgcggtcgtcgggatgagcttactggcg |
| | | ttgatatcgatcttcgcgtcgtgctacatgctggttgcggcccgcagtaag |
| | | tgcttgaccccttatgctttaacaccaggagctgcagttccgtggacgctg |
| | | gggatactctgctgcgccccgcgggcgcacgcagctagtgtggcagagact |
| | | atggcctacttgtgggaccaaaaccaagcgttgttctggttggagtttgcg |
| | | gcccctgttgcctgcatcctcatcatcacgtattgcctcagaaacgtgctg |
| | | tgttgctgtaagagcctttcttttttagtgctactgagcctcggggcaacc |
| | | gccagagcttacgaacattcgacagtaatgccgaacgtggtggggttcccg |
| | | tataaggctcacattgaaaggccaggatatagccccctcactttgcagatg |
| | | caggttgttgaaaccagcctcgaaccaacccttaatttggaatacataacc |
| | | tgtgagtacaagacggtcgtcccgtcgccgtacgtgaagtgctgcggcgcc |
| | | tcagagtgctccactaaagagaagcctgactaccaatgcaaggtttacaca |
| | | ggcgtgtacccgttcatgtggggaggggcatattgcttctgcgactcagaa |
| | | aacacgcaactcagcgaggcgtacgtcgatcgatcggacgtatgcaggcat |
| | | gatcacgcatctgcttacaaagcccatacagcatcgctgaaggccaaagtg |
| | | agggttatgtacggcaacgtaaaccagactgtggatgtttacgtgaacgga |
| | | gaccatgccgtcacgatagggggtactcagttcatattcgggccgctgtca |
| | | tcggcctggaccccgttcgacaacaagatagtcgtgtacaaagacgaagtg |
| | | ttcaatcaggacttcccgccgtacggatctgggcaaccagggcgcttcggc |
| | | gacatccaaagcagaacagtggagagtaacgacctgtacgcgaacacggca |
| | | ctgaagctggcacgcccttcacccggcatggtccatgtaccgtacacacag |
| | | acaccttcagggttcaaatattggctaaaggaaaaagggacagccctaaat |
| | | acgaaggctccttttggctgccaaatcaaaacgaaccctgtcagggccatg |
| | | aactgcgccgtgggaaacatccctgtctccatgaatttgcctgacagcgcc |
| | | tttacccgcattgtcgaggcgccgaccatcattgacctgacttgcacagtg |
| | | gctacctgtacgcactcctcggatttcggcggcgtcttgacactgacgtac |
| | | aagaccgacaagaacggggactgctctgtacactcgcactctaacgtagct |
| | | actctacaggaggccacagcaaaagtgaagacagcaggtaaggtgacctta |
| | | cacttctccacggcaagcgcatcaccttcttttgtggtgtcgctatgcagt |
| | | gctagggccacctgttcagcgtcgtgtgagcccccgaaagaccacatagtc |
| | | ccatatgcggctagccacagtaacgtagtgtttccagacatgtcgggcacc |
| | | gcactatcatgggtgcagaaaatctcgggtggtctgggggccttcgcaatc |
| | | ggcgctatcctggtgctggttgtggtcacttgcattgggctccgcagataa |
| | | gctcgctttcttgctgtccaatttctattaaaggttcctttgttccctaag |
| | | tccaactactaaactgggggatattatgaagggccttgagcatctggattc |
| | | tgcctaataaaaaacatttattttcattgcaa |
| Nucleic Acid | 86 | aaaaaaaaaacgcggaagagccgcgtgataacgcaggaaagaacatgtgag |
| Template, | | caaaaggccagcaaaaggccaggaaccgtaaaaaggccgcgttgctggcgt |
| Sam002- | | ttttccataggctccgcccccctgacgagcatcacaaaaatcgacgctcaa |
| mouse IL-12, | | gtcagaggtggcgaaacccgacaggactataaagataccaggcgtttcccc |
| kanamycin | | ctggaagctccctcgtgcgctctcctgttccgaccctgccgcttaccggat |
| resistance) | | acctgtccgcctttctcccttcgggaagcgtggcgctttctcatagctcac |
| (SVP-327)- | | gctgtaggtatctcagttcggtgtaggtcgttcgctccaagctgggctgtg |
| the GOI | | tgcacgaaccccccgttcagcccgaccgctgcgccttatccggtaactatc |
| (mouse IL-12) | | gtcttgagtccaacccggtaagacacgacttatcgccactggcagcagcca |
| is bolded. The | | ctggtaacaggattagcagagcgaggtatgtaggcggtgctacagagttct |
| GOI can be | | tgaagtggtggcctaactacggctacactagaagaacagtatttggtatct |
| substituted | | gcgctctgctgaagccagttaccttcggaaaaagagttggtagctcttgat |
| with one or | | ccggcaaacaaaccaccgctggtagcggtggtttttttgtttgcaagcagc |
| more different | | agattacgcgcagaaaaaaaggatctcaagaagatcctttgatcttttcta |
| GOIs. | | cggggtctgacgctcagtggaacgaaaactcacgttaagggattttggtca |
| | | tgagattatcaaaaaggatcttcacctagatccttttaaattaaaaatgaa |
| | | gttttaaatcaatctaaagtatatatgagtaacctgaggctatggcagggc |
| | | ctgccgccccgacgttggctgcgagccctgggccttcacccgaacttgggg |
| | | ggtggggtggggaaaaggaagaaacgcgggcgtattggccccaatggggtc |
| | | tcggtggggtatcgacagagtgccagccctgggaccgaaccccgcgtttat |
| | | gaacaaacgacccaacaccgtgcgttttattctgtctttttattgccgtca |
| | | tagcgcgggttccttccggtattgtctccttccgtgtttcagttagcctcc |
| | | ccctagggtgggcgaagaactccagcatgagatccccgcgctggaggatca |
| | | tccagccggcgtcccggaaaacgattccgaagcccaacctttcatagaagg |
| | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggactggacctggcgaatac |
| | | tgttcttggttgccgccgctacagggactcacgcaatgtgggagcttgaaa |
| | | aagacgtctatgtagtagaagtggactggacacctgatgctcctggcgaga |
| | | cagttaacctcacatgcgatacccctgaggaagatgatatcacctggactt |
| | | ctgaccagagacacggggtgattgggagcggcaaaaccctgacgatcactg |
| | | tgaaggagtttctggacgccggccagtatacctgtcacaaggggggggaga |
| | | ccctgagtcatagccacctgttgctgcacaagaaggagaatggcatctggt |
| | | ctacagagatcctgaagaactttaagaacaagaccttcctgaagtgtgaag |
| | | caccaaactacagtggtcgctttacctgcagctggctggtccaaagaaaca |
| | | tggacctgaaatttaatataaagagtagttcctcgagtcctgattccaggg |
| | | ccgtgacgtgcggcatggcaagcctttcagccgaaaaagtcacgctggatc |
| | | agcgagactatgagaagtacagcgttagctgtcaggaggacgtaacttgcc |
| | | cgactgccgaggagactctgcccatagagctcgctctggaggccaggcagc |
| | | agaacaaatatgagaattacagcactagtttctttattagagacatcatca |
| | | aacccgacccacccaagaatctgcagatgaagccgctgaagaatagtcagg |
| | | tcgaggtttcctgggaatatccagattcatggtccactccgcattcttatt |
| | | tttccttaaaattctttgttaggattcagcggaaaaaagaaaagatgaaag |
| | | agacggaggaagggtgcaaccagaagggggccttcctggtggaaaagacaa |
| | | gcactgaggtccaatgtaagggtgggaacgtttgcgtgcaggctcaggatc |
| | | gctactacaacagcagttgctctaagtgggcctgcgtaccttgtcgcgtca |
| | | ggagtggaggggggtcagggggtggctcaggcggcggcagtgggggcagca |
| | | gggtgatcccagtgtctgggccggcccgttgcttgtctcaatccagaaacc |
| | | tcctcaagaccactgacgatatggtaaagactgcccgagagaagctaaaac |
| | | actactcttgtacagctgaagatatagaccatgaggatataacacgggacc |
| | | agacctctactctgaaaacctgtctgcctcttgagctgcacaagaacgagt |
| | | cctgtctggctacccgcgaaacctcaagcacaaccagaggtagttgcctgc |
| | | ccccacaaaagacatcgcttatgatgaccttgtgtctgggatctatttatg |
| | | aggacctgaagatgtaccaaactgagttccaggcaataaatgctgctctcc |
| | | agaatcacaatcatcaacaaatcatccttgataaggggatgctggtcgcaa |
| | | tcgacgagctcatgcaatcgctgaaccacaatggggaaaccctcaggcaga |
| | | aaccaccggtgggagaggccgacccctaccgtgttaaaatgaagttgtgta |
| | | ttcttttgcatgcattctctacaagagtcgttaccatcaatcgcgtcatgg |
| | | ggtacctgtcatcagcctagcatcgatgatatcgcggccgcatacagcagc |
| | | aattggcaagctgcttacatagaactcgcggcgattggcatgccgccttaa |
| | | aatttttattttatttttcttttcttttccgaatcggattttgtttttaat |
| | | atttcaaaaaaaaaaaaaaaa |
| Nucleic Acid | 87 | aaaaaaaaaacgcggaagagccgcgtgataacgcaggaaagaacatgtgag |
| Template | | caaaaggccagcaaaaggccaggaaccgtaaaaaggccgcgttgctggcgt |
| Sam002- | | ttttccataggctccgcccccctgacgagcatcacaaaaatcgacgctcaa |
| human IL-12, | | gtcttgagtccaacccggtaagacacgacttatcgccactggcagcagcca |
| kanamycin | | ctggtaacaggattagcagagcgaggtatgtaggcggtgctacagagttct |
| resistance | | acctgtccgcctttctcccttcgggaagcgtggcgctttctcatagctcac |
| (SVP-315)- | | gctgtaggtatctcagttcggtgtaggtcgttcgctccaagctgggctgtg |
| the GOI | | tgcacgaaccccccgttcagcccgaccgctgcgccttatccggtaactatc |
| (human IL-12) | | gtcagaggtggcgaaacccgacaggactataaagataccaggcgtttcccc |
| is bolded. The | | ctggaagctccctcgtgcgctctcctgttccgaccctgccgcttaccggat |
| GOI can be | | tgaagtggtggcctaactacggctacactagaagaacagtatttggtatct |
| substituted | | gcgctctgctgaagccagttaccttcggaaaaagagttggtagctcttgat |
| with one or | | ccggcaaacaaaccaccgctggtagcggtggtttttttgtttgcaagcagc |
| more different | | agattacgcgcagaaaaaaaggatctcaagaagatcctttgatcttttcta |
| GOIs. | | cggggtctgacgctcagtggaacgaaaactcacgttaagggattttggtca |
| | | tgagattatcaaaaaggatcttcacctagatccttttaaattaaaaatgaa |
| | | gttttaaatcaatctaaagtatatatgagtaacctgaggctatggcagggc |
| | | ctgccgccccgacgttggctgcgagccctgggccttcacccgaacttgggg |
| | | ggtggggtggggaaaaggaagaaacgcgggcgtattggccccaatggggtc |
| | | tcggtggggtatcgacagagtgccagccctgggaccgaaccccgcgtttat |
| | | gaacaaacgacccaacaccgtgcgttttattctgtctttttattgccgtca |
| | | tagcgcgggttccttccggtattgtctccttccgtgtttcagttagcctcc |
| | | ccctagggtgggcgaagaactccagcatgagatccccgcgctggaggatca |
| | | tccagccggcgtcccggaaaacgattccgaagcccaacctttcatagaagg |
| | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggactggacctggcgaatac |
| | | tgttcttggttgccgccgctacagggactcacgcaatatgggagctgaaga |
| | | aggacgtgtatgtcgtggagctggactggtacccagatgctcctggcgaaa |
| | | tggtggttttaacatgtgatacccccgaggaggacggcatcacatggactc |
| | | tggaccagagttctgaggtgctggggtccggcaagactctgacaatccagg |
| | | ttaaggagttcggcgacgcaggacagtacacttgtcacaagggaggtgagg |
| | | tgctttctcacagcctgttgctgctccataagaaggaagacggtatttggt |
| | | caaccgacatcctcaaggaccagaaggagcccaaaaacaagacctttctga |
| | | gatgtgaggccaagaattacagcggtagattcacttgttggtggctcacca |
| | | ccatatccacagacttgaccttcagtgtcaaaagttcacgagggagctcag |
| | | atcctcaaggcgttacctgtggcgcagcgacgctgtccgcagaaagagtca |
| | | ggggagacaacaaggaatacgagtactctgtcgagtgccaggaggattccg |
| | | cctgtccggccgcagaggagtctttacctattgaggtgatggtcgatgccg |
| | | tgcacaagcttaagtacgagaattacacatcaagttttttcatccgcgaca |
| | | tcattaaacctgatccaccaaagaacctgcagctcaagcctctgaagaata |
| | | gcaggcaggtcgaggtaagctgggagtatcctgatacctggtccacccccc |
| | | acagttatttcagcctcaccttctgcgtccaagtccagggaaagagcaaga |
| | | gagagaagaaggatagggtgttcacagataagacttcagctactgtgatct |
| | | gcagaaagaatgcgtctatctctgtgcgagcacaagacaggtactacagtt |
| | | ctagctggagcgagtgggcatcagtcccctgcagtggtggcggaagcggag |
| | | ggggcagcggaggtgggagcggagggagcaggaacctcccagttgctacac |
| | | ctgacccgggaatgtttccatgcctccaccattcccagaatctcctccgag |
| | | ccgtgtccaatatgctgcaaaaggctcggcagaccttggagttttaccctt |
| | | gcacctcagaagaaatcgatcatgaggatatcacaaaggataagacgagca |
| | | ctgttgaggcatgcctgcccctggagctaactaagaatgagtcttgcctga |
| | | acagcagggagacttccttcattaccaacggtagctgtcttgccagcagga |
| | | agacatcttttatgatggccctgtgtctatctagcatatatgaagacctga |
| | | agatgtaccaggtggaattcaaaaccatgaatgctaagcttctcatggatc |
| | | ccaagaggcaaatcttcctggaccagaatatgcttgctgtcatagatgaac |
| | | tgatgcaggcgttgaattttaacagcgagacggtgcctcaaaaaagctcac |
| | | tggaagaacctgatttttataaaacgaagatcaagctgtgtattttactac |
| | | acgcctttagaatccgcgctgttaccatcgacagagtaatgtcctacctaa |
| | | atgcttcatagcatcgatgatatcgcggccgcatacagcagcaattggcaa |
| | | gctgcttacatagaactcgcggcgattggcatgccgccttaaaatttttat |
| | | tttatttttcttttcttttccgaatcggattttgtttttaatatttcaaaa |
| | | aaaaaaaaaaaa |
| Second nucleic | 88 | gctcgctttcttgctgtccaatttctattaaaggttcctttgttccctaag |
| acid construct | | tccaactactaaactgggggatattatgaagggccttgagcatctggattc |
| template, | | tgcctaataaaaaacatttattttcattgcaaagccacaccctggagctag |
| modified | | caaaaaaaaaaaaaaaaaaaaaaaaaaaaaagcatatgactaaaaaaaaaa |
| mRNA | | aaaaaaaaaaaaaaaaaaaaaaaaaaaaaaaaaaaaaaaaaaaaaaaaaaa |
| encoding with | | aaaaaaaaacgcgtatcgatgatatcgcggccgcatacagcagcaattggc |
| capsids/envelop | | aagctgcttacatagaactcgcggcgattggcatgccgccttaaaattttt |
| from SIN, | | attttatttttcttttcttttccgaatcggattttgtttttaatatttcaa |
| Amp resistance | | aaaaaaaaaaaaaaaaaaaaaaaacgcgtgataacgcaggaaagaacatgt |
| (SVP-464)- | | gagcaaaaggccagcaaaaggccaggaaccgtaaaaaggccgcgttgctgg |
| the viral | | cgtttttccataggctccgcccccctgacgagcatcacaaaaatcgacgct |
| structural | | caagtcagaggtggcgaaacccgacaggactataaagataccaggcgtttc |
| protein of SIN | | cccctggaagctccctcgtgcgctctcctgttccgaccctgccgcttaccg |
| is bolded. The | | gatacctgtccgcctttctcccttcgggaagcgtggcgctttctcatagct |
| bolded | | cacgctgtaggtatctcagttcggtgtaggtcgttcgctccaagctgggct |
| sequence can | | gtgtgcacgaaccccccgttcagcccgaccgctgcgccttatccggtaact |
| be substituted | | atcgtcttgagtccaacccggtaagacacgacttatcgccactggcagcag |
| with one or | | ccactggtaacaggattagcagagcgaggtatgtaggcggtgctacagagt |
| more different | | tcttgaagtggtggcctaactacggctacactagaagaacagtatttggta |
| viral structural | | tctgcgctctgctgaagccagttaccttcggaaaaagagttggtagctctt |
| proteins. | | gatccggcaaacaaaccaccgctggtagcggtttttttgtttgcaagcagc |
| | | agattacgcgcagaaaaaaaggatctcaagaagatcctttgatcttttcta |
| | | cggggtctgacgctcagtggaacgaaaactcacgttaagggattttggtca |
| | | tgagattatcaaaaaggatcttcacctagatccttttaaattaaaaatgaa |
| | | gttttaaatcaatctaaagtatatatgagtaaacttggtctgacagttacc |
| | | aatgcttaatcagtgaggcacctatctcagcgatctgtctatttcgttcat |
| | | ccatagttgcctgactccccgtcgtgtagataactacgatacgggagggct |
| | | taccatctggccccagtgctgcaatgataccgcgagacccacgctcaccgg |
| | | ctccagatttatcagcaataaaccagccagccggaagggccgagcgcagaa |
| | | gtggtcctgcaactttatccgcctccatccagtctattaattgttgccggg |
| | | aagctagagtaagtagttcgccagttaatagtttgcgcaacgttgttgcca |
| | | ttgctacaggcatcgtggtgtcacgctcgtcgtttggtatggcttcattca |
| | | gctccggttcccaacgatcaaggcgagttacatgatcccccatgttgtgca |
| | | aaaaagcggttagctccttcggtcctccgatcgttgtcagaagtaagttgg |
| | | ccgcagtgttatcactcatggttatggcagcactgcataattctcttactg |
| | | tcatgccatccgtaagatgcttttctgtgactggtgagtactcaaccaagt |
| | | cattctgagaatagtgtatgcggcgaccgagttgctcttgcccggcgtcaa |
| | | tacgggataataccgcgccacatagcagaactttaaaagtgctcatcattg |
| | | gaaaacgttcttcggggcgaaaactctcaaggatcttaccgctgttgagat |
| | | ccagttcgatgtaacccactcgtgcacccaactgatcttcagcatctttta |
| | | ctttcaccagcgtttctgggtgagcaaaaacaggaaggcaaaatgccgcaa |
| | | aaaagggaataagggcgacacggaaatgttgaatactcatactcttccttt |
| | | ttcaatattattgaagcatttatcagggttattgtctcatgagcggataca |
| | | tatttgaatgtatttagaaaaataaacaaataggggttccgcgcacatttc |
| | | cccgaaaagtgccacctgagctctaatacgactcactataggataggacat |
| | | ttgcttctgacacaactgtgttcactagcaacctcaaacagacaccatgaa |
| | | tagaggaatatttaacatgctgagccgccgccccttccccgcccccacagc |
| | | tatgtggaggcctaggcgaaggaggcaggcggcaccactgcccgcccgtaa |
| | | tgggttggcatcccaaattcaacagctaaccacagctgtcagtgccctagt |
| | | cattggtcaagccacccgacctcaacaaccaaggccacgcccgctaccacg |
| | | ccctaagaagcagtccccgaagcaaccaccgaatgctaaaaagacaaaacc |
| | | gcaggagaacaaggagaaacaacccgcaaaaccgaagccgggtaagagaca |
| | | gcgtatggcactaaaattgatgctgacaggactctttgacgtgaacaacga |
| | | agccggtgacgtaataggacacgccctcgctatggaaggcaaagtgatgaa |
| | | gccacttcacgtgaaaggggtcatagaccacccggttttggcaaagctgaa |
| | | atttaccaagtcatcagcgtacgacatggagttcgcccagcttccagccag |
| | | tatgaaaagcgaagcatttgcatacaccagcgagcaccctgaaggcttcta |
| | | caactggcatcatggtgccgtgcagtacagcggcggtcgctttacagtccc |
| | | gcgcggagttgggggaaaaggcgacagtggaagaccgatcatggataatac |
| | | aggtaaggtggtcgcgatagtgctgggcggcgctgacgaaggcgcccgcac |
| | | tgctctctccgtcgtcacttggaacagcaaaggaaagaccatcaaaacgac |
| | | tccggaagggactgaggaatggtctgcagcaccattagtcacggctatgtg |
| | | cttctttgagaatgtatcgttcccgtgcagtcgtccgcctacgtgctattc |
| | | ccgaaaaccatctcgtgcccttgacatcctggaggagaacgtgaacaacga |
| | | cgcctacgataccttattagcggcagtgttgaagtgcaggacgtccggtcg |
| | | gaacaaacgcagcatcaccgacgatttcaccctgaccagcccgtacctggg |
| | | cacttgctcgtattgccatcatactgaaccctgcttcagcccaattaagat |
| | | cgagcaagtttgggacgaggcggacgacggatcaatacgaatacagacatc |
| | | cgcccagttcgggtacgaccaaagtggggcagcagacgtcaacaagtacag |
| | | gtacatgtcaattgaccaggaccatgccgtgaaagaaggctctatggatca |
| | | tatcaagatcagcacatccgggccctgtaggcggcttcaatcacaaaggta |
| | | cttcttgcttgcgaaatgcccaccgggagatagcgttacggttagcataac |
| | | cggcggcactatggccacctcctgtacattagcacgtaaaataaggcaaaa |
| | | attcgtaggacgtgagaagtatgaccttcccccagcacacggaaagaacat |
| | | cccctgcaccgtgtatgatcgtcttaaggagacgtcagccggatacatcac |
| | | catgcacaggcctggacctcatgcctatacctcatacctagaggctacctc |
| | | aggaaaaatttacgccaaaccaccatcaggaaagaacatcacgtacgagtg |
| | | taagtgtggcgattataaaactgcgaccgtttccgtcagaacagagatcgc |
| | | aggatgtactgccataaaacagtgcgtggcgtacaagagcgaccagaccaa |
| | | gtgggtgttcaactcgcccgatctaatcaggcacgctgaccacacagccca |
| | | aggtaaattccactttcctttcaagcctaccttcagtacatgcctggtacc |
| | | actggcacacgagccgactgtaattcatgggttcaaacacataagcctcca |
| | | tttagacacagaccacccaaccttgctcaccattagaagactgggcgagaa |
| | | acccgaaccgacgtcggagtggatctcaggaaaaacagttaggaattttac |
| | | tgtcgacagagacgggctagagtatatatggggcaatcacgaaccggtgag |
| | | agtatatgcacaagagtccgcccccggtgaccctcacggatggccacacaa |
| | | aataattcaacactactatcaacgccacccagtatacactagcttggctgt |
| | | agcagcttccgctgtggcggtgatagtcggcctggccaccgccgtgatgtg |
| | | tatctgcagagcgcgacgggagtgcctaaccccgtacgcgttagccccgaa |
| | | tgcagtcatcccgacgtccttggccttattgtgctgcgttcggtccgccaa |
| | | cgccgaatctttcaccgacacgatgggatacctatggtcgaacagtcaaac |
| | | atttttctgggtgcagctgtgcatccccttggcagtagtaatagtactcgt |
| | | acgctgctgttcgtgctgcctcccttttttagtggttgcaggcgcctacct |
| | | ggcgaaggtagacgccttcgaacatgcgaccactgtcccaaatgtgccgaa |
| | | gattccgtataaagctttggtcgagagagccggatacgccccgcttaactt |
| | | ggaaattactgttatgtcttcagaggtcctcccttcgattcaccaagaata |
| | | catcacctgcaaatactccactatcgtgccttccccgaaagtgaaatgctg |
| | | cggttcccttgagtgccaacccgcttcgcatgccgactacaactgcaaagt |
| | | atttggcggcgtttaccctttcatgtggggtggagcgcaatgtttttgcga |
| | | cagcgagaacagccagatgagtgaggcgtatgtcgagctatcggccgattg |
| | | tgcccttgatcacgcacaggcggtgaaagtgcacacagccgcaatgaaggt |
| | | cggactacgcattgtgtatgggaacaccactagcctcctggatgcgtacgt |
| | | aaacggagtcacgccaggcacatctaaagacctgaaagtcattgccggacc |
| | | catctcgtccgcgttttccccttttgactctaaagtcgtgatacaccgagg |
| | | gctcgtgtacaattacgattttccagagtacggggcaatgaaaccgggtgc |
| | | ctttggggacattcaagctagctctttgacgggcaacgacctgattgccag |
| | | caccgacatacgtttgcttaaacccgcagcaaagaatgtgcacgttcccta |
| | | tacccaagcagcatcaggatttgaaatttggaagaacaactccggaagacc |
| | | actgcaagagacggccccatttggctgtaagattgccgtcaaccctctgaa |
| | | ggccgtggattgttcctatgggaacatcccgatatcgattgatatccccca |
| | | cgccgcctttattagaccatccgatgcgccatttatctccgaagtgaagtg |
| | | cgaagtcagcgactgcacatactctgctgacttcggaggcatggccaccct |
| | | acagtatgtgtctgaccgagagggccagtgcccagtgcattcccattcaag |
| | | taccgccacactccaggaatcaactgttcacgtcttggaaaaaggggcgac |
| | | gacagtgcacttcagcacagctagccctcacgccagctttgtggtctcatt |
| | | atgcggaaagaagacgacctgcaccgcaggatgcaaaccacctgtcggcca |
| | | catagtcagcacaccccacaagaaggaccaagagttccagaccgccgtctc |
| | | caggacatcatggagctggctcttcgcgctgttcggaggagcttcgtccct |
| | | agtaatcatgggactgttaatcttctcctgcagcatgatgctcactaacac |
| | | cagaagatga |
| Second nucleic | 89 | gctcgctttcttgctgtccaatttctattaaaggttcctttgttccctaag |
| acid construct | | tccaactactaaactgggggatattatgaagggccttgagcatctggattc |
| template- | | tgcctaataaaaaacatttattttcattgcaaagccacaccctggagctag |
| modified | | caaaaaaaaaaaaaaaaaaaaaaaaaaaaaaaaaaaaaaaaaaaaaaaacg |
| mRNA | | cgtatcgatgatatcgcggccgcatacagcagcaattggcaagctgcttac |
| encoding with | | atagaactcgcggcgattggcatgccgccttaaaatttttattttattttt |
| capsids/envelop | | cttttcttttccgaatcggattttgtttttaatatttcaaaaaaaaaaaaa |
| from VEE, | | aaaaaaaaaaaaacgcgtgataacgcaggaaagaacatgtgagcaaaaggc |
| Amp | | cagcaaaaggccaggaaccgtaaaaaggccgcgttgctggcgtttttccat |
| resistance) | | aggctccgcccccctgacgagcatcacaaaaatcgacgctcaagtcagagg |
| (SVP-365) | | tggcgaaacccgacaggactataaagataccaggcgtttccccctggaagc |
| | | tccctcgtgcgctctcctgttccgaccctgccgcttaccggatacctgtcc |
| | | gcctttctcccttcgggaagcgtggcgctttctcatagctcacgctgtagg |
| | | tatctcagttcggtgtaggtcgttcgctccaagctgggctgtgtgcacgaa |
| | | ccccccgttcagcccgaccgctgcgccttatccggtaactatcgtcttgag |
| | | tccaacccggtaagacacgacttatcgccactggcagcagccactggtaac |
| | | aggattagcagagcgaggtatgtaggcggtgctacagagttcttgaagtgg |
| | | tggcctaactacggctacactagaagaacagtatttggtatctgcgctctg |
| | | ctgaagccagttaccttcggaaaaagagttggtagctcttgatccggcaaa |
| | | caaaccaccgctggtagcggtttttttgtttgcaagcagcagattacgcgc |
| | | agaaaaaaaggatctcaagaagatcctttgatcttttctacggggtctgac |
| | | gctcagtggaacgaaaactcacgttaagggattttggtcatgagattatca |
| | | aaaaggatcttcacctagatccttttaaattaaaaatgaagttttaaatca |
| | | atctaaagtatatatgagtaaacttggtctgacagttaccaatgcttaatc |
| | | agtgaggcacctatctcagcgatctgtctatttcgttcatccatagttgcc |
| | | tgactccccgtcgtgtagataactacgatacgggagggcttaccatctggc |
| | | cccagtgctgcaatgataccgcgagacccacgctcaccggctccagattta |
| | | tcagcaataaaccagccagccggaagggccgagcgcagaagtggtcctgca |
| | | actttatccgcctccatccagtctattaattgttgccgggaagctagagta |
| | | agtagttcgccagttaatagtttgcgcaacgttgttgccattgctacaggc |
| | | atcgtggtgtcacgctcgtcgtttggtatggcttcattcagctccggttcc |
| | | caacgatcaaggcgagttacatgatcccccatgttgtgcaaaaaagcggtt |
| | | agctccttcggtcctccgatcgttgtcagaagtaagttggccgcagtgtta |
| | | tcactcatggttatggcagcactgcataattctcttactgtcatgccatcc |
| | | gtaagatgcttttctgtgactggtgagtactcaaccaagtcattctgagaa |
| | | tagtgtatgcggcgaccgagttgctcttgcccggcgtcaatacgggataat |
| | | accgcgccacatagcagaactttaaaagtgctcatcattggaaaacgttct |
| | | tcggggcgaaaactctcaaggatcttaccgctgttgagatccagttcgatg |
| | | taacccactcgtgcacccaactgatcttcagcatcttttactttcaccagc |
| | | gtttctgggtgagcaaaaacaggaaggcaaaatgccgcaaaaaagggaata |
| | | agggcgacacggaaatgttgaatactcatactcttcctttttcaatattat |
| | | tgaagcatttatcagggttattgtctcatgagcggatacatatttgaatgt |
| | | atttagaaaaataaacaaataggggttccgcgcacatttccccgaaaagtg |
| | | ccacctgagctctaatacgactcactataggataggacatttgcttctgac |
| | | acaactgtgttcactagcaacctcaaacagacaccatgttcccgttccagc |
| | | caatgtatccgatgcagccaatgccctatcgcaacccgttcgcggccccgc |
| | | gcaggccctggttccccagaaccgacccttttctggcgatgcaggtgcagg |
| | | aattaacccgctcgatggctaacctgacgttcaagcaacgccgggacgcgc |
| | | cacctgaggggccatccgctaagaaaccgaagaaggaggcctcgcaaaaac |
| | | agaaagggggaggccaagggaagaagaagaagaaccaagggaagaagaagg |
| | | ctaagacagggccgcctaatccgaaggcacagaatggaaacaagaagaaga |
| | | ccaacaagaaaccaggcaagagacagcgcatggtcatgaaattggaatctg |
| | | acaagacgttcccaatcatgttggaagggaagataaacggctacgcttgtg |
| | | tggtcggagggaagttattcaggccgatgcatgtggaaggcaagatcgaca |
| | | acgacgttctggccgcgcttaagacgaagaaagcatccaaatacgatcttg |
| | | agtatgcagatgtgccacagaacatgcgggccgatacattcaaatacaccc |
| | | atgagaaaccccaaggctattacagctggcatcatggagcagtccaatatg |
| | | aaaatgggcgtttcacggtgccgaaaggagttggggccaagggagacagcg |
| | | gacgacccattctggataaccagggacgggtggtcgctattgtgctgggag |
| | | gtgtgaatgaaggatctaggacagccctttcagtcgtcatgtggaacgaga |
| | | agggagttaccgtgaagtatactccggagaactgcgagcaatggtcactag |
| | | tgaccaccatgtgtctgctcgccaatgtgacgttcccatgtgctcaaccac |
| | | caatttgctacgacagaaaaccagcagagactttggccatgctcagcgtta |
| | | acgttgacaacccgggctacgatgagctgctggaagcagctgttaagtgcc |
| | | ccggaaggaaaaggagatccaccgaggagctgtttaatgagtataagctaa |
| | | cgcgcccttacatggccagatgcatcagatgtgcagttgggagctgccata |
| | | gtccaatagcaatcgaggcagtaaagagcgacgggcacgacggttatgtta |
| | | gacttcagacttcctcgcagtatggcctggattcctccggcaacttaaagg |
| | | gcaggaccatgcggtatgacatgcacgggaccattaaagagataccactac |
| | | atcaagtgtcactctatacatctcgcccgtgtcacattgtggatgggcacg |
| | | gttatttcctgcttgccaggtgcccggcaggggactccatcaccatggaat |
| | | ttaagaaagattccgtcagacactcctgctcggtgccgtatgaagtgaaat |
| | | ttaatcctgtaggcagagaactctatactcatcccccagaacacggagtag |
| | | agcaagcgtgccaagtctacgcacatgatgcacagaacagaggagcttatg |
| | | tcgagatgcacctcccgggctcagaagtggacagcagtttggtttccttga |
| | | gcggcagttcagtcaccgtgacacctcctgatgggactagcgccctggtgg |
| | | aatgcgagtgtggcggcacaaagatctccgagaccatcaacaagacaaaac |
| | | agttcagccagtgcacaaagaaggagcagtgcagagcatatcggctgcaga |
| | | acgataagtgggtgtataattctgacaaactgcccaaagcagcgggagcca |
| | | ccttaaaaggaaaactgcatgtcccattcttgctggcagacggcaaatgca |
| | | ccgtgcctctagcaccagaacctatgataaccttcggtttcagatcagtgt |
| | | cactgaaactgcaccctaagaatcccacatatctaatcacccgccaacttg |
| | | ctgatgagcctcactacacgcacgagctcatatctgaaccagctgttagga |
| | | attttaccgtcaccgaaaaagggtgggagtttgtatggggaaaccacccgc |
| | | cgaaaaggttttgggcacaggaaacagcacccggaaatccacatgggctac |
| | | cgcacgaggtgataactcattattaccacagataccctatgtccaccatcc |
| | | tgggtttgtcaatttgtgccgccattgcaaccgtttccgttgcagcgtcta |
| | | cctggctgttttgcagatctagagttgcgtgcctaactccttaccggctaa |
| | | cacctaacgctaggataccattttgtctggctgtgctttgctgcgcccgca |
| | | ctgcccgggccgagaccacctgggagtccttggatcacctatggaacaata |
| | | accaacagatgttctggattcaattgctgatccctctggccgccttgatcg |
| | | tagtgactcgcctgctcaggtgcgtgtgctgtgtcgtgccttttttagtca |
| | | tggccggcgccgcaggcgccggcgcctacgagcacgcgaccacgatgccga |
| | | gccaagcgggaatctcgtataacactatagtcaacagagcaggctacgcac |
| | | cactccctatcagcataacaccaacaaagatcaagctgatacctacagtga |
| | | acttggagtacgtcacctgccactacaaaacaggaatggattcaccagcca |
| | | tcaaatgctgcggatctcaggaatgcactccaacttacaggcctgatgaac |
| | | agtgcaaagtcttcacaggggtttacccgttcatgtggggtggtgcatatt |
| | | gcttttgcgacactgagaacacccaagtcagcaaggcctacgtaatgaaat |
| | | ctgacgactgccttgcggatcatgctgaagcatataaagcgcacacagcct |
| | | cagtgcaggcgttcctcaacatcacagtgggagaacactctattgtgacta |
| | | ccgtgtatgtgaatggagaaactcctgtgaatttcaatggggtcaaaataa |
| | | ctgcaggtccgctttccacagcttggacaccctttgatcgcaaaatcgtgc |
| | | agtatgccggggagatctataattatgattttcctgagtatggggcaggac |
| | | aaccaggagcatttggagatatacaatccagaacagtctcaagctctgatc |
| | | tgtatgccaataccaacctagtgctgcagagacccaaagcaggagcgatcc |
| | | acgtgccatacactcaggcaccttcgggttttgagcaatggaagaaagata |
| | | aagctccatcattgaaatttaccgcccctttcggatgcgaaatatatacaa |
| | | accccattcgcgccgaaaactgtgctgtagggtcaattccattagcctttg |
| | | acattcccgacgccttgttcaccagggtgtcagaaacaccgacactttcag |
| | | cggccgaatgcactcttaacgagtgcgtgtattcttccgactttggtggga |
| | | tcgccacggtcaagtactcggccagcaagtcaggcaagtgcgcagtccatg |
| | | tgccatcagggactgctaccctaaaagaagcagcagtcgagctaaccgagc |
| | | aagggtcggcgactatccatttctcgaccgcaaatatccacccggagttca |
| | | ggctccaaatatgcacatcatatgttacgtgcaaaggtgattgtcaccccc |
| | | cgaaagaccatattgtgacacaccctcagtatcacgcccaaacatttacag |
| | | ccgcggtgtcaaaaaccgcgtggacgtggttaacatccctgctgggaggat |
| | | cagccgtaattattataattggcttggtgctggctactattgtggccatgt |
| | | acgtgctgaccaaccagaaacataattga |
| Nucleic Acid | 90 | aaaaaaaaaacgcgtgataacgcaggaaagaacatgtgagcaaaaggccag |
| Template, | | caaaaggccaggaaccgtaaaaaggccgcgttgctggcgtttttccatagg |
| Sam002-full | | ctccgcccccctgacgagcatcacaaaaatcgacgctcaagtcagaggtgg |
| plasmids of | | cgaaacccgacaggactataaagataccaggcgtttccccctggaagctcc |
| self-amplifying | | ctcgtgcgctctcctgttccgaccctgccgcttaccggatacctgtccgcc |
| mRNA | | tttctcccttcgggaagcgtggcgctttctcatagctcacgctgtaggtat |
| encoding | | ctcagttcggtgtaggtcgttcgctccaagctgggctgtgtgcacgaaccc |
| mouse wild | | cccgttcagcccgaccgctgcgccttatccggtaactatcgtcttgagtcc |
| type IL-18, | | aacccggtaagacacgacttatcgccactggcagcagccactggtaacagg |
| Amp | | attagcagagcgaggtatgtaggcggtgctacagagttcttgaagtggtgg |
| resistance) | | cctaactacggctacactagaagaacagtatttggtatctgcgctctgctg |
| (SVP-305)- | | aagccagttaccttcggaaaaagagttggtagctcttgatccggcaaacaa |
| the GOI | | accaccgctggtagcggtttttttgtttgcaagcagcagattacgcgcaga |
| (mouse IL-18) | | aaaaaaggatctcaagaagatcctttgatcttttctacggggtctgacgct |
| is bolded. The | | cagtggaacgaaaactcacgttaagggattttggtcatgagattatcaaaa |
| GOI can be | | aggatcttcacctagatccttttaaattaaaaatgaagttttaaatcaatc |
| substituted | | taaagtatatatgagtaaacttggtctgacagttaccaatgcttaatcagt |
| with one or | | gaggcacctatctcagcgatctgtctatttcgttcatccatagttgcctga |
| more different | | ctccccgtcgtgtagataactacgatacgggagggcttaccatctggcccc |
| GOIs. | | agtgctgcaatgataccgcgagacccacgctcaccggctccagatttatca |
| | | gcaataaaccagccagccggaagggccgagcgcagaagtggtcctgcaact |
| | | ttatccgcctccatccagtctattaattgttgccgggaagctagagtaagt |
| | | agttcgccagttaatagtttgcgcaacgttgttgccattgctacaggcatc |
| | | gtggtgtcacgctcgtcgtttggtatggcttcattcagctccggttcccaa |
| | | cgatcaaggcgagttacatgatcccccatgttgtgcaaaaaagcggttagc |
| | | tccttcggtcctccgatcgttgtcagaagtaagttggccgcagtgttatca |
| | | ctcatggttatggcagcactgcataattctcttactgtcatgccatccgta |
| | | agatgcttttctgtgactggtgagtactcaaccaagtcattctgagaatag |
| | | tgtatgcggcgaccgagttgctcttgcccggcgtcaatacgggataatacc |
| | | gcgccacatagcagaactttaaaagtgctcatcattggaaaacgttcttcg |
| | | gggcgaaaactctcaaggatcttaccgctgttgagatccagttcgatgtaa |
| | | cccactcgtgcacccaactgatcttcagcatcttttactttcaccagcgtt |
| | | tctgggtgagcaaaaacaggaaggcaaaatgccgcaaaaaagggaataagg |
| | | gcgacacggaaatgttgaatactcatactcttcctttttcaatattattga |
| | | agcatttatcagggttattgtctcatgagcggatacatatttgaatgtatt |
| | | tagaaaaataaacaaataggggttccgcgcacatttccccgaaaagtgcca |
| | | cctgagctctaatacgactcactataggataggcggcgcatgagagaagcc |
| | | cagaccaattacctacccaaaatggagaaagttcacgttgacatcgaggaa |
| | | gacagcccattcctcagagctttgcagcggagcttcccgcagtttgaggta |
| | | gaagccaagcaggtcactgataatgaccatgctaatgccagagcgttttcg |
| | | catctggcttcaaaactgatcgaaacggaggtggacccatccgacacgatc |
| | | cttgacattggaagtgcgcccgcccgcagaatgtattctaagcacaagtat |
| | | cattgtatctgtccgatgagatgtgcggaagatccggacagattgtataag |
| | | tatgcaactaagctgaagaaaaactgtaaggaaataactgataaggaattg |
| | | gacaagaaaatgaaggagctggccgccgtcatgagcgaccctgacctggaa |
| | | actgagactatgtgcctccacgacgacgagtcgtgtcgctacgaagggcaa |
| | | gtcgctgtttaccaggatgtatacgcggttgacggaccgacaagtctctat |
| | | caccaagccaataagggagttagagtcgcctactggataggctttgacacc |
| | | accccttttatgtttaagaacttggctggagcatatccatcatactctacc |
| | | aactgggccgacgaaaccgtgttaacggctcgtaacataggcctatgcagc |
| | | tctgacgttatggagcggtcacgtagagggatgtccattcttagaaagaag |
| | | tatttgaaaccatccaacaatgttctattctctgttggctcgaccatctac |
| | | cacgagaagagggacttactgaggagctggcacctgccgtctgtatttcac |
| | | ttacgtggcaagcaaaattacacatgtcggtgtgagactatagttagttgc |
| | | gacgggtacgtcgttaaaagaatagctatcagtccaggcctgtatgggaag |
| | | ccttcaggctatgctgctacgatgcaccgcgagggattcttgtgctgcaaa |
| | | gtgacagacacattgaacggggagagggtctcttttcccgtgtgcacgtat |
| | | gtgccagctacattgtgtgaccaaatgactggcatactggcaacagatgtc |
| | | agtgcggacgacgcgcaaaaactgctggttgggctcaaccagcgtatagtc |
| | | gtcaacggtcgcacccagagaaacaccaataccatgaaaaattaccttttg |
| | | cccgtagtggcccaggcatttgctaggtgggcaaaggaatataaggaagat |
| | | caagaagatgaaaggccactaggactacgagatagacagttagtcatgggg |
| | | tgttgttgggcttttagaaggcacaagataacatctatttataagcgcccg |
| | | gatacccaaaccatcatcaaagtgaacagcgatttccactcattcgtgctg |
| | | cccaggataggcagtaacacattggagatcgggctgagaacaagaatcagg |
| | | aaaatgttagaggagcacaaggagccgtcacctctcattaccgccgaggac |
| | | gtacaagaagctaagtgcgcagccgatgaggctaaggaggtgcgtgaagcc |
| | | gaggagttgcgcgcagctctaccacctttggcagctgatgttgaggagccc |
| | | actctggaagccgatgtcgacttgatgttacaagaggctggggccggctca |
| | | gtggagacacctcgtggcttgataaaggttaccagctacgatggcgaggac |
| | | aagatcggctcttacgctgtgctttctccgcaggctgtactcaagagtgaa |
| | | aaattatcttgcatccaccctctcgctgaacaagtcatagtgataacacac |
| | | tctggccgaaaagggcgttatgccgtggaaccataccatggtaaagtagtg |
| | | gtgccagagggacatgcaatacccgtccaggactttcaagctctgagtgaa |
| | | agtgccaccattgtgtacaacgaacgtgagttcgtaaacaggtacctgcac |
| | | catattgccacacatggaggagcgctgaacactgatgaagaatattacaaa |
| | | actgtcaagcccagcgagcacgacggcgaatacctgtacgacatcgacagg |
| | | aaacagtgcgtcaagaaagaactagtcactgggctagggctcacaggcgag |
| | | ctggtggatcctcccttccatgaattcgcctacgagagtctgagaacacga |
| | | ccagccgctccttaccaagtaccaaccataggggtgtatggcgtgccagga |
| | | tcaggcaagtctggcatcattaaaagcgcagtcaccaaaaaagatctagtg |
| | | gtgagcgccaagaaagaaaactgtgcagaaattataagggacgtcaagaaa |
| | | atgaaagggctggacgtcaatgccagaactgtggactcagtgctcttgaat |
| | | ggatgcaaacaccccgtagagaccctgtatattgacgaagcttttgcttgt |
| | | catgcaggtactctcagagcgctcatagccattataagacctaaaaaggca |
| | | gtgctctgcggggatcccaaacagtgcggtttttttaacatgatgtgcctg |
| | | aaagtgcattttaaccacgagatttgcacacaagtcttccacaaaagcatc |
| | | tctcgccgttgcactaaatctgtgacttcggtcgtctcaaccttgttttac |
| | | gacaaaaaaatgagaacgacgaatccgaaagagactaagattgtgattgac |
| | | actaccggcagtaccaaacctaagcaggacgatctcattctcacttgtttc |
| | | agagggtgggtgaagcagttgcaaatagattacaaaggcaacgaaataatg |
| | | acggcagctgcctctcaagggctgacccgtaaaggtgtgtatgccgttcgg |
| | | tacaaggtgaatgaaaatcctctgtacgcacccacctcagaacatgtgaac |
| | | gtcctactgacccgcacggaggaccgcatcgtgtggaaaacactagccggc |
| | | gacccatggataaaaacactgactgccaagtaccctgggaatttcactgcc |
| | | acgatagaggagtggcaagcagagcatgatgccatcatgaggcacatcttg |
| | | gagagaccggaccctaccgacgtcttccagaataaggcaaacgtgtgttgg |
| | | gccaaggctttagtgccggtgctgaagaccgctggcatagacatgaccact |
| | | gaacaatggaacactgtggattattttgaaacggacaaagctcactcagca |
| | | gagatagtattgaaccaactatgcgtgaggttctttggactcgatctggac |
| | | tccggtctattttctgcacccactgttccgttatccattaggaataatcac |
| | | tgggataactccccgtcgcctaacatgtacgggctgaataaagaagtggtc |
| | | cgtcagctctctcgcaggtacccacaactgcctcgggcagttgccactgga |
| | | agagtctatgacatgaacactggtacactgcgcaattatgatccgcgcata |
| | | aacctagtacctgtaaacagaagactgcctcatgctttagtcctccaccat |
| | | aatgaacacccacagagtgacttttcttcattcgtcagcaaattgaagggc |
| | | agaactgtcctggtggtcggggaaaagttgtccgtcccaggcaaaatggtt |
| | | gactggttgtcagaccggcctgaggctaccttcagagctcggctggattta |
| | | ggcatcccaggtgatgtgcccaaatatgacataatatttgttaatgtgagg |
| | | accccatataaataccatcactatcagcagtgtgaagaccatgccattaag |
| | | cttagcatgttgaccaagaaagcttgtctgcatctgaatcccggcggaacc |
| | | tgtgtcagcataggttatggttacgctgacagggccagcgaaagcatcatt |
| | | ggtgctatagcgcggcagttcaagttttcccgggtatgcaaaccgaaatcc |
| | | tcacttgaagagacggaagttctgtttgtattcattgggtacgatcgcaag |
| | | gcccgtacgcacaattcttacaagctttcatcaaccttgaccaacatttat |
| | | acaggttccagactccacgaagccggatgtgcaccctcatatcatgtggtg |
| | | cgaggggatattgccacggccaccgaaggagtgattataaatgctgctaac |
| | | agcaaaggacaacctggcggaggggtgtgcggagcgctgtataagaaattc |
| | | ccggaaagcttcgatttacagccgatcgaagtaggaaaagcgcgactggtc |
| | | aaaggtgcagctaaacatatcattcatgccgtaggaccaaacttcaacaaa |
| | | gtttcggaggttgaaggtgacaaacagttggcagaggcttatgagtccatc |
| | | gctaagattgtcaacgataacaattacaagtcagtagcgattccactgttg |
| | | tccaccggcatcttttccgggaacaaagatcgactaacccaatcattgaac |
| | | catttgctgacagctttagacaccactgatgcagatgtagccatatactgc |
| | | agggacaagaaatgggaaatgactctcaaggaagcagtggctaggagagaa |
| | | gcagtggaggagatatgcatatccgacgactcttcagtgacagaacctgat |
| | | gcagagctggtgagggtgcatccgaagagttctttggctggaaggaagggc |
| | | tacagcacaagcgatggcaaaactttctcatatttggaagggaccaagttt |
| | | caccaggcggccaaggatatagcagaaattaatgccatgtggcccgttgca |
| | | acggaggccaatgagcaggtatgcatgtatatcctcggagaaagcatgagc |
| | | agtattaggtcgaaatgccccgtcgaagagtcggaagcctccacaccacct |
| | | agcacgctgccttgcttgtgcatccatgccatgactccagaaagagtacag |
| | | cgcctaaaagcctcacgtccagaacaaattactgtgtgctcatcctttcca |
| | | ttgccgaagtatagaatcactggtgtgcagaagatccaatgctcccagcct |
| | | atattgttctcaccgaaagtgcctgcgtatattcatccaaggaagtatctc |
| | | gtggaaacaccaccggtagacgagactccggagccatcggcagagaaccaa |
| | | tccacagaggggacacctgaacaaccaccacttataaccgaggatgagacc |
| | | aggactagaacgcctgagccgatcatcatcgaagaggaagaagaggatagc |
| | | ataagtttgctgtcagatggcccgacccaccaggtgctgcaagtcgaggca |
| | | gacattcacgggccgccctctgtatctagctcatcctggtccattcctcat |
| | | gcatccgactttgatgtggacagtttatccatacttgacaccctggaggga |
| | | gctagcgtgaccagcggggcaacgtcagccgagactaactcttacttcgca |
| | | aagagtatggagtttctggcgcgaccggtgcctgcgcctcgaacagtattc |
| | | aggaaccctccacatcccgctccgcgcacaagaacaccgtcacttgcaccc |
| | | agcagggcctgctcgagaaccagcctagtttccaccccgccaggcgtgaat |
| | | agggtgatcactagagaggagctcgaggcgcttaccccgtcacgcactcct |
| | | agcaggtcggtctcgagaaccagcctggtctccaacccgccaggcgtaaat |
| | | agggtgattacaagagaggagtttgaggcgttcgtagcacaacaacaatga |
| | | cggtttgatgcgggtgcatacatcttttcctccgacaccggtcaagggcat |
| | | ttacaacaaaaatcagtaaggcaaacggtgctatccgaagtggtgttggag |
| | | aggaccgaattggagatttcgtatgccccgcgcctcgaccaagaaaaagaa |
| | | gaattactacgcaagaaattacagttaaatcccacacctgctaacagaagc |
| | | agataccagtccaggaaggtggagaacatgaaagccataacagctagacgt |
| | | attctgcaaggcctagggcattatttgaaggcagaaggaaaagtggagtgc |
| | | taccgaaccctgcatcctgttcctttgtattcatctagtgtgaaccgtgcc |
| | | ttttcaagccccaaggtcgcagtggaagcctgtaacgccatgttgaaagag |
| | | aactttccgactgtggcttcttactgtattattccagagtacgatgcctat |
| | | ttggacatggttgacggagcttcatgctgcttagacactgccagtttttgc |
| | | cctgcaaagctgcgcagctttccaaagaaacactcctatttggaacccaca |
| | | atacgatcggcagtgccttcagcgatccagaacacgctccagaacgtcctg |
| | | gcagctgccacaaaaagaaattgcaatgtcacgcaaatgagagaattgccc |
| | | gtattggattcggcggcctttaatgtggaatgcttcaagaaatatgcgtgt |
| | | aataatgaatattgggaaacgtttaaagaaaaccccatcaggcttactgaa |
| | | gaaaacgtggtaaattacattaccaaattaaaaggaccaaaagctgctgct |
| | | ctttttgcgaagacacataatttgaatatgttgcaggacataccaatggac |
| | | aggtttgtaatggacttaaagagagacgtgaaagtgactccaggaacaaaa |
| | | catactgaagaacggcccaaggtacaggtgatccaggctgccgatccgcta |
| | | gcaacagcgtatctgtgcggaatccaccgagagctggttaggagattaaat |
| | | gcggtcctgcttccgaacattcatacactgtttgatatgtcggctgaagac |
| | | tttgacgctattatagccgagcacttccagcctggggattgtgttctggaa |
| | | actgacatcgcgtcgtttgataaaagtgaggacgacgccatggctctgacc |
| | | gcgttaatgattctggaagacttaggtgtggacgcagagctgttgacgctg |
| | | attgaggcggctttcggcgaaatttcatcaatacatttgcccactaaaact |
| | | aaatttaaattcggagccatgatgaaatctggaatgttcctcacactgttt |
| | | gtgaacacagtcattaacattgtaatcgcaagcagagtgttgagagaacgg |
| | | ctaaccggatcaccatgtgcagcattcattggagatgacaatatcgtgaaa |
| | | ggagtcaaatcggacaaattaatggcagacaggtgcgccacctggttgaat |
| | | atggaagtcaagattatagatgctgtggtgggcgagaaagcgccttatttc |
| | | tgtggagggtttattttgtgtgactccgtgaccggcacagcgtgccgtgtg |
| | | gcagaccccctaaaaaggctgtttaagcttggcaaacctctggcagcagac |
| | | gatgaacatgatgatgacaggagaagggcattgcatgaagagtcaacacgc |
| | | tggaaccgagtgggtattctttcagagctgtgcaaggcagtagaatcaagg |
| | | tatgaaaccgtaggaacttccatcatagttatggccatgactactctagct |
| | | agcagtgttaaatcattcagctacctgagaggggcccctataactctctac |
| | | ggctaacctgaatggactacgacatagtctagtccgccaagttcgaaggcg |
| | | cgcctctagagccaccatgacctcccggcttgtgagggtactggctgctgc |
| | | tatgctggtggctgctgctgtgagtgtggcaaactttgggagactccactg |
| | | taccactgccgttatacggaatataaacgatcaggtcctctttgtagacaa |
| | | gagacagccagttttcgaggacatgactgatatagatcagagtgcctctga |
| | | gcctcaaactcggctcataatatacatgtataaggatagcgaggttcgggg |
| | | acttgccgtgactttgtccgtgaaagactcaaaaatgagcaccctgtcatg |
| | | caaaaataagataatttctttcgaggaaatggaccctcccgagaacattga |
| | | tgatatccagtccgacttgatatttttccagaaacgggttcccggtcataa |
| | | caaaatggagtttgagtcttcattgtacgagggtcattttttggcatgcca |
| | | gaaggaggatgatgccttcaaactgattttgaagaaaaaagacgagaacgg |
| | | agacaagtctgtcatgtttacacttaccaaccttcatcaatcctagcatcg |
| | | atgatatcgcggccgcatacagcagcaattggcaagctgcttacatagaac |
| | | tcgcggcgattggcatgccgccttaaaatttttattttatttttcttttct |
| | | tttccgaatcggattttgtttttaatatttcaaaaaaaaaaaaaaaa |
| Nucleic Acid | 91 | aaaaaaaaaacgcgtgataacgcaggaaagaacatgtgagcaaaaggccag |
| Template, | | caaaaggccaggaaccgtaaaaaggccgcgttgctggcgtttttccatagg |
| Sam002-full | | ctccgcccccctgacgagcatcacaaaaatcgacgctcaagtcagaggtgg |
| plasmids of | | cgaaacccgacaggactataaagataccaggcgtttccccctggaagctcc |
| self-amplifying | | ctcgtgcgctctcctgttccgaccctgccgcttaccggatacctgtccgcc |
| mRNA | | tttctcccttcgggaagcgtggcgctttctcatagctcacgctgtaggtat |
| encoding | | ctcagttcggtgtaggtcgttcgctccaagctgggctgtgtgcacgaaccc |
| mouse mutant | | cccgttcagcccgaccgctgcgccttatccggtaactatcgtcttgagtcc |
| IL-18, Amp | | aacccggtaagacacgacttatcgccactggcagcagccactggtaacagg |
| resistance) | | attagcagagcgaggtatgtaggcggtgctacagagttcttgaagtggtgg |
| (SVP-306)- | | cctaactacggctacactagaagaacagtatttggtatctgcgctctgctg |
| the GOI | | aagccagttaccttcggaaaaagagttggtagctcttgatccggcaaacaa |
| (mouse mutant | | accaccgctggtagcggtttttttgtttgcaagcagcagattacgcgcaga |
| IL-12) is | | aaaaaaggatctcaagaagatcctttgatcttttctacggggtctgacgct |
| bolded. The | | cagtggaacgaaaactcacgttaagggattttggtcatgagattatcaaaa |
| GOI can be | | aggatcttcacctagatccttttaaattaaaaatgaagttttaaatcaatc |
| substituted | | taaagtatatatgagtaaacttggtctgacagttaccaatgcttaatcagt |
| with one or | | gaggcacctatctcagcgatctgtctatttcgttcatccatagttgcctga |
| more different | | ctccccgtcgtgtagataactacgatacgggagggcttaccatctggcccc |
| GOIs. | | agtgctgcaatgataccgcgagacccacgctcaccggctccagatttatca |
| | | gcaataaaccagccagccggaagggccgagcgcagaagtggtcctgcaact |
| | | ttatccgcctccatccagtctattaattgttgccgggaagctagagtaagt |
| | | agttcgccagttaatagtttgcgcaacgttgttgccattgctacaggcatc |
| | | gtggtgtcacgctcgtcgtttggtatggcttcattcagctccggttcccaa |
| | | cgatcaaggcgagttacatgatcccccatgttgtgcaaaaaagcggttagc |
| | | tccttcggtcctccgatcgttgtcagaagtaagttggccgcagtgttatca |
| | | ctcatggttatggcagcactgcataattctcttactgtcatgccatccgta |
| | | agatgcttttctgtgactggtgagtactcaaccaagtcattctgagaatag |
| | | tgtatgcggcgaccgagttgctcttgcccggcgtcaatacgggataatacc |
| | | gcgccacatagcagaactttaaaagtgctcatcattggaaaacgttcttcg |
| | | gggcgaaaactctcaaggatcttaccgctgttgagatccagttcgatgtaa |
| | | cccactcgtgcacccaactgatcttcagcatcttttactttcaccagcgtt |
| | | tctgggtgagcaaaaacaggaaggcaaaatgccgcaaaaaagggaataagg |
| | | gcgacacggaaatgttgaatactcatactcttcctttttcaatattattga |
| | | agcatttatcagggttattgtctcatgagcggatacatatttgaatgtatt |
| | | tagaaaaataaacaaataggggttccgcgcacatttccccgaaaagtgcca |
| | | cctgagctctaatacgactcactataggataggcggcgcatgagagaagcc |
| | | cagaccaattacctacccaaaatggagaaagttcacgttgacatcgaggaa |
| | | gacagcccattcctcagagctttgcagcggagcttcccgcagtttgaggta |
| | | gaagccaagcaggtcactgataatgaccatgctaatgccagagcgttttcg |
| | | catctggcttcaaaactgatcgaaacggaggtggacccatccgacacgatc |
| | | cttgacattggaagtgcgcccgcccgcagaatgtattctaagcacaagtat |
| | | cattgtatctgtccgatgagatgtgcggaagatccggacagattgtataag |
| | | tatgcaactaagctgaagaaaaactgtaaggaaataactgataaggaattg |
| | | gacaagaaaatgaaggagctggccgccgtcatgagcgaccctgacctggaa |
| | | actgagactatgtgcctccacgacgacgagtcgtgtcgctacgaagggcaa |
| | | gtcgctgtttaccaggatgtatacgcggttgacggaccgacaagtctctat |
| | | caccaagccaataagggagttagagtcgcctactggataggctttgacacc |
| | | accccttttatgtttaagaacttggctggagcatatccatcatactctacc |
| | | aactgggccgacgaaaccgtgttaacggctcgtaacataggcctatgcagc |
| | | tctgacgttatggagcggtcacgtagagggatgtccattcttagaaagaag |
| | | tatttgaaaccatccaacaatgttctattctctgttggctcgaccatctac |
| | | cacgagaagagggacttactgaggagctggcacctgccgtctgtatttcac |
| | | ttacgtggcaagcaaaattacacatgtcggtgtgagactatagttagttgc |
| | | gacgggtacgtcgttaaaagaatagctatcagtccaggcctgtatgggaag |
| | | ccttcaggctatgctgctacgatgcaccgcgagggattcttgtgctgcaaa |
| | | gtgacagacacattgaacggggagagggtctcttttcccgtgtgcacgtat |
| | | gtgccagctacattgtgtgaccaaatgactggcatactggcaacagatgtc |
| | | agtgcggacgacgcgcaaaaactgctggttgggctcaaccagcgtatagtc |
| | | gtcaacggtcgcacccagagaaacaccaataccatgaaaaattaccttttg |
| | | cccgtagtggcccaggcatttgctaggtgggcaaaggaatataaggaagat |
| | | caagaagatgaaaggccactaggactacgagatagacagttagtcatgggg |
| | | tgttgttgggcttttagaaggcacaagataacatctatttataagcgcccg |
| | | gatacccaaaccatcatcaaagtgaacagcgatttccactcattcgtgctg |
| | | cccaggataggcagtaacacattggagatcgggctgagaacaagaatcagg |
| | | aaaatgttagaggagcacaaggagccgtcacctctcattaccgccgaggac |
| | | gtacaagaagctaagtgcgcagccgatgaggctaaggaggtgcgtgaagcc |
| | | gaggagttgcgcgcagctctaccacctttggcagctgatgttgaggagccc |
| | | actctggaagccgatgtcgacttgatgttacaagaggctggggccggctca |
| | | gtggagacacctcgtggcttgataaaggttaccagctacgatggcgaggac |
| | | aagatcggctcttacgctgtgctttctccgcaggctgtactcaagagtgaa |
| | | aaattatcttgcatccaccctctcgctgaacaagtcatagtgataacacac |
| | | tctggccgaaaagggcgttatgccgtggaaccataccatggtaaagtagtg |
| | | gtgccagagggacatgcaatacccgtccaggactttcaagctctgagtgaa |
| | | agtgccaccattgtgtacaacgaacgtgagttcgtaaacaggtacctgcac |
| | | catattgccacacatggaggagcgctgaacactgatgaagaatattacaaa |
| | | actgtcaagcccagcgagcacgacggcgaatacctgtacgacatcgacagg |
| | | aaacagtgcgtcaagaaagaactagtcactgggctagggctcacaggcgag |
| | | ctggtggatcctcccttccatgaattcgcctacgagagtctgagaacacga |
| | | ccagccgctccttaccaagtaccaaccataggggtgtatggcgtgccagga |
| | | tcaggcaagtctggcatcattaaaagcgcagtcaccaaaaaagatctagtg |
| | | gtgagcgccaagaaagaaaactgtgcagaaattataagggacgtcaagaaa |
| | | atgaaagggctggacgtcaatgccagaactgtggactcagtgctcttgaat |
| | | ggatgcaaacaccccgtagagaccctgtatattgacgaagcttttgcttgt |
| | | catgcaggtactctcagagcgctcatagccattataagacctaaaaaggca |
| | | gtgctctgcggggatcccaaacagtgcggtttttttaacatgatgtgcctg |
| | | aaagtgcattttaaccacgagatttgcacacaagtcttccacaaaagcatc |
| | | tctcgccgttgcactaaatctgtgacttcggtcgtctcaaccttgttttac |
| | | gacaaaaaaatgagaacgacgaatccgaaagagactaagattgtgattgac |
| | | actaccggcagtaccaaacctaagcaggacgatctcattctcacttgtttc |
| | | agagggtgggtgaagcagttgcaaatagattacaaaggcaacgaaataatg |
| | | acggcagctgcctctcaagggctgacccgtaaaggtgtgtatgccgttcgg |
| | | tacaaggtgaatgaaaatcctctgtacgcacccacctcagaacatgtgaac |
| | | gtcctactgacccgcacggaggaccgcatcgtgtggaaaacactagccggc |
| | | gacccatggataaaaacactgactgccaagtaccctgggaatttcactgcc |
| | | acgatagaggagtggcaagcagagcatgatgccatcatgaggcacatcttg |
| | | gagagaccggaccctaccgacgtcttccagaataaggcaaacgtgtgttgg |
| | | gccaaggctttagtgccggtgctgaagaccgctggcatagacatgaccact |
| | | gaacaatggaacactgtggattattttgaaacggacaaagctcactcagca |
| | | gagatagtattgaaccaactatgcgtgaggttctttggactcgatctggac |
| | | tccggtctattttctgcacccactgttccgttatccattaggaataatcac |
| | | tgggataactccccgtcgcctaacatgtacgggctgaataaagaagtggtc |
| | | cgtcagctctctcgcaggtacccacaactgcctcgggcagttgccactgga |
| | | agagtctatgacatgaacactggtacactgcgcaattatgatccgcgcata |
| | | aacctagtacctgtaaacagaagactgcctcatgctttagtcctccaccat |
| | | aatgaacacccacagagtgacttttcttcattcgtcagcaaattgaagggc |
| | | agaactgtcctggtggtcggggaaaagttgtccgtcccaggcaaaatggtt |
| | | gactggttgtcagaccggcctgaggctaccttcagagctcggctggattta |
| | | ggcatcccaggtgatgtgcccaaatatgacataatatttgttaatgtgagg |
| | | accccatataaataccatcactatcagcagtgtgaagaccatgccattaag |
| | | cttagcatgttgaccaagaaagcttgtctgcatctgaatcccggcggaacc |
| | | tgtgtcagcataggttatggttacgctgacagggccagcgaaagcatcatt |
| | | ggtgctatagcgcggcagttcaagttttcccgggtatgcaaaccgaaatcc |
| | | tcacttgaagagacggaagttctgtttgtattcattgggtacgatcgcaag |
| | | gcccgtacgcacaattcttacaagctttcatcaaccttgaccaacatttat |
| | | acaggttccagactccacgaagccggatgtgcaccctcatatcatgtggtg |
| | | cgaggggatattgccacggccaccgaaggagtgattataaatgctgctaac |
| | | agcaaaggacaacctggcggaggggtgtgcggagcgctgtataagaaattc |
| | | ccggaaagcttcgatttacagccgatcgaagtaggaaaagcgcgactggtc |
| | | aaaggtgcagctaaacatatcattcatgccgtaggaccaaacttcaacaaa |
| | | gtttcggaggttgaaggtgacaaacagttggcagaggcttatgagtccatc |
| | | gctaagattgtcaacgataacaattacaagtcagtagcgattccactgttg |
| | | tccaccggcatcttttccgggaacaaagatcgactaacccaatcattgaac |
| | | catttgctgacagctttagacaccactgatgcagatgtagccatatactgc |
| | | agggacaagaaatgggaaatgactctcaaggaagcagtggctaggagagaa |
| | | gcagtggaggagatatgcatatccgacgactcttcagtgacagaacctgat |
| | | gcagagctggtgagggtgcatccgaagagttctttggctggaaggaagggc |
| | | tacagcacaagcgatggcaaaactttctcatatttggaagggaccaagttt |
| | | caccaggcggccaaggatatagcagaaattaatgccatgtggcccgttgca |
| | | acggaggccaatgagcaggtatgcatgtatatcctcggagaaagcatgagc |
| | | agtattaggtcgaaatgccccgtcgaagagtcggaagcctccacaccacct |
| | | agcacgctgccttgcttgtgcatccatgccatgactccagaaagagtacag |
| | | cgcctaaaagcctcacgtccagaacaaattactgtgtgctcatcctttcca |
| | | ttgccgaagtatagaatcactggtgtgcagaagatccaatgctcccagcct |
| | | atattgttctcaccgaaagtgcctgcgtatattcatccaaggaagtatctc |
| | | gtggaaacaccaccggtagacgagactccggagccatcggcagagaaccaa |
| | | tccacagaggggacacctgaacaaccaccacttataaccgaggatgagacc |
| | | aggactagaacgcctgagccgatcatcatcgaagaggaagaagaggatagc |
| | | ataagtttgctgtcagatggcccgacccaccaggtgctgcaagtcgaggca |
| | | gacattcacgggccgccctctgtatctagctcatcctggtccattcctcat |
| | | gcatccgactttgatgtggacagtttatccatacttgacaccctggaggga |
| | | gctagcgtgaccagcggggcaacgtcagccgagactaactcttacttcgca |
| | | aagagtatggagtttctggcgcgaccggtgcctgcgcctcgaacagtattc |
| | | aggaaccctccacatcccgctccgcgcacaagaacaccgtcacttgcaccc |
| | | agcagggcctgctcgagaaccagcctagtttccaccccgccaggcgtgaat |
| | | agggtgatcactagagaggagctcgaggcgcttaccccgtcacgcactcct |
| | | agcaggtcggtctcgagaaccagcctggtctccaacccgccaggcgtaaat |
| | | agggtgattacaagagaggagtttgaggcgttcgtagcacaacaacaatga |
| | | cggtttgatgcgggtgcatacatcttttcctccgacaccggtcaagggcat |
| | | ttacaacaaaaatcagtaaggcaaacggtgctatccgaagtggtgttggag |
| | | aggaccgaattggagatttcgtatgccccgcgcctcgaccaagaaaaagaa |
| | | gaattactacgcaagaaattacagttaaatcccacacctgctaacagaagc |
| | | agataccagtccaggaaggtggagaacatgaaagccataacagctagacgt |
| | | attctgcaaggcctagggcattatttgaaggcagaaggaaaagtggagtgc |
| | | taccgaaccctgcatcctgttcctttgtattcatctagtgtgaaccgtgcc |
| | | ttttcaagccccaaggtcgcagtggaagcctgtaacgccatgttgaaagag |
| | | aactttccgactgtggcttcttactgtattattccagagtacgatgcctat |
| | | ttggacatggttgacggagcttcatgctgcttagacactgccagtttttgc |
| | | cctgcaaagctgcgcagctttccaaagaaacactcctatttggaacccaca |
| | | atacgatcggcagtgccttcagcgatccagaacacgctccagaacgtcctg |
| | | gcagctgccacaaaaagaaattgcaatgtcacgcaaatgagagaattgccc |
| | | gtattggattcggcggcctttaatgtggaatgcttcaagaaatatgcgtgt |
| | | aataatgaatattgggaaacgtttaaagaaaaccccatcaggcttactgaa |
| | | gaaaacgtggtaaattacattaccaaattaaaaggaccaaaagctgctgct |
| | | ctttttgcgaagacacataatttgaatatgttgcaggacataccaatggac |
| | | aggtttgtaatggacttaaagagagacgtgaaagtgactccaggaacaaaa |
| | | catactgaagaacggcccaaggtacaggtgatccaggctgccgatccgcta |
| | | gcaacagcgtatctgtgcggaatccaccgagagctggttaggagattaaat |
| | | gcggtcctgcttccgaacattcatacactgtttgatatgtcggctgaagac |
| | | tttgacgctattatagccgagcacttccagcctggggattgtgttctggaa |
| | | actgacatcgcgtcgtttgataaaagtgaggacgacgccatggctctgacc |
| | | gcgttaatgattctggaagacttaggtgtggacgcagagctgttgacgctg |
| | | attgaggcggctttcggcgaaatttcatcaatacatttgcccactaaaact |
| | | aaatttaaattcggagccatgatgaaatctggaatgttcctcacactgttt |
| | | gtgaacacagtcattaacattgtaatcgcaagcagagtgttgagagaacgg |
| | | ctaaccggatcaccatgtgcagcattcattggagatgacaatatcgtgaaa |
| | | ggagtcaaatcggacaaattaatggcagacaggtgcgccacctggttgaat |
| | | atggaagtcaagattatagatgctgtggtgggcgagaaagcgccttatttc |
| | | tgtggagggtttattttgtgtgactccgtgaccggcacagcgtgccgtgtg |
| | | gcagaccccctaaaaaggctgtttaagcttggcaaacctctggcagcagac |
| | | gatgaacatgatgatgacaggagaagggcattgcatgaagagtcaacacgc |
| | | tggaaccgagtgggtattctttcagagctgtgcaaggcagtagaatcaagg |
| | | tatgaaaccgtaggaacttccatcatagttatggccatgactactctagct |
| | | agcagtgttaaatcattcagctacctgagaggggcccctataactctctac |
| | | ggctaacctgaatggactacgacatagtctagtccgccaagttcgaaggcg |
| | | cgcctctagagccaccatgacctcccggcttgtgagggtactggctgctgc |
| | | tatgctggtggctgctgctgtgagtgtggcacacttcggacgactccattg |
| | | tactacagccgttatacgaaatatcaatgatcaggtgttgtttgtagacaa |
| | | aaggcagcccgtgtttgaagacatgactgatattgatcagagtgcctctga |
| | | gccccagactagactgattatatatgcttatggcgactctagagctagagg |
| | | gaaagcagtaactctgtcagtgaaagacagcaagatgtctaccctgtcttg |
| | | caagaataaaataatctcatttgaggagatggacccccctgaaaacataga |
| | | tgacatccaaagtgacctgatattctttcagaagcgggtacccggtcataa |
| | | caaaatggagttcgagtcaagcctctatgagggtcactttctcgcctgcca |
| | | aaaggaagatgacgccttcaaattgatcttgaaaaaaaaagacgagaatgg |
| | | tgataagtctgtcatgttcacactcactaatttgcatcagtcatagcatcg |
| | | atgatatcgcggccgcatacagcagcaattggcaagctgcttacatagaac |
| | | tcgcggcgattggcatgccgccttaaaatttttattttatttttcttttct |
| | | tttccgaatcggattttgtttttaatatttcaaaaaaaaaaaaaaaa |
| Nucleic Acid | 92 | aaaaaaaaaacgcggaagagccgcgtgataacgcaggaaagaacatgtgag |
| Template- | | caaaaggccagcaaaaggccaggaaccgtaaaaaggccgcgttgctggcgt |
| Sam002-full | | ttttccataggctccgcccccctgacgagcatcacaaaaatcgacgctcaa |
| plasmids of | | gtcagaggtggcgaaacccgacaggactataaagataccaggcgtttcccc |
| self-amplifying | | ctggaagctccctcgtgcgctctcctgttccgaccctgccgcttaccggat |
| mRNA | | acctgtccgcctttctcccttcgggaagcgtggcgctttctcatagctcac |
| encoding | | gctgtaggtatctcagttcggtgtaggtcgttcgctccaagctgggctgtg |
| human wild | | tgcacgaaccccccgttcagcccgaccgctgcgccttatccggtaactatc |
| type IL-18, | | gtcttgagtccaacccggtaagacacgacttatcgccactggcagcagcca |
| kana | | ctggtaacaggattagcagagcgaggtatgtaggcggtgctacagagttct |
| resistance) | | tgaagtggtggcctaactacggctacactagaagaacagtatttggtatct |
| (SVP-479)- | | gcgctctgctgaagccagttaccttcggaaaaagagttggtagctcttgat |
| the GOI | | ccggcaaacaaaccaccgctggtagcggtggtttttttgtttgcaagcagc |
| (human IL-18) | | agattacgcgcagaaaaaaaggatctcaagaagatcctttgatcttttcta |
| is bolded. The | | cggggtctgacgctcagtggaacgaaaactcacgttaagggattttggtca |
| GOI can be | | tgagattatcaaaaaggatcttcacctagatccttttaaattaaaaatgaa |
| substituted | | gttttaaatcaatctaaagtatatatgagtaacctgaggctatggcagggc |
| with one or | | ctgccgccccgacgttggctgcgagccctgggccttcacccgaacttgggg |
| more different | | ggtggggtggggaaaaggaagaaacgcgggcgtattggccccaatggggtc |
| GOIs. | | tcggtggggtatcgacagagtgccagccctgggaccgaaccccgcgtttat |
| | | gaacaaacgacccaacaccgtgcgttttattctgtctttttattgccgtca |
| | | tagcgcgggttccttccggtattgtctccttccgtgtttcagttagcctcc |
| | | ccctagggtgggcgaagaactccagcatgagatccccgcgctggaggatca |
| | | tccagccggcgtcccggaaaacgattccgaagcccaacctttcatagaagg |
| | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggacatgagagtcccagctc |
| | | aactgcttggtcttcttctgctgtggttgcgcggtgccagatgttactttg |
| | | gaaagcttgagtccaaactttctgtcataagaaacttgaacgaccaggtgt |
| | | tgtttatagaccagggtaatcgacctctcttcgaggatatgacagatagcg |
| | | actgcagagataacgccccccgcaccatattcataatatccatgtataagg |
| | | actcacaaccaagaggcatggccgtaacaataagcgtaaaatgtgagaaaa |
| | | tctctaccctgagctgtgagaacaagatcataagcttcaaggaaatgaacc |
| | | caccagacaacatcaaggatactaaaagtgacattatcttcttccaacgga |
| | | gcgtcccaggccacgacaacaagatgcagttcgagtccagcagttacgaag |
| | | gttacttcctggcgtgcgagaaagagcgcgacctttttaagctcattctga |
| | | agaaggaagatgagttgggtgacaggagcattatgttcacggtccagaatg |
| | | aggactagcatcgatgatatcgcggccgcatacagcagcaattggcaagct |
| | | gcttacatagaactcgcggcgattggcatgccgccttaaaatttttatttt |
| | | atttttcttttcttttccgaatcggattttgtttttaatatttcaaaaaaa |
| | | aaaaaaaaa |
| Nucleic Acid | 93 | aaaaaaaaaacgcggaagagccgcgtgataacgcaggaaagaacatgtgag |
| Template, | | caaaaggccagcaaaaggccaggaaccgtaaaaaggccgcgttgctggcgt |
| Sam002-full | | ttttccataggctccgcccccctgacgagcatcacaaaaatcgacgctcaa |
| plasmids of | | gtcagaggtggcgaaacccgacaggactataaagataccaggcgtttcccc |
| self-amplifying | | ctggaagctccctcgtgcgctctcctgttccgaccctgccgcttaccggat |
| mRNA | | acctgtccgcctttctcccttcgggaagcgtggcgctttctcatagctcac |
| encoding | | gctgtaggtatctcagttcggtgtaggtcgttcgctccaagctgggctgtg |
| human wild | | tgcacgaaccccccgttcagcccgaccgctgcgccttatccggtaactatc |
| mutant IL-18, | | gtcttgagtccaacccggtaagacacgacttatcgccactggcagcagcca |
| kana resistance | | ctggtaacaggattagcagagcgaggtatgtaggcggtgctacagagttct |
| (SVP-408)- | | tgaagtggtggcctaactacggctacactagaagaacagtatttggtatct |
| the GOI | | gcgctctgctgaagccagttaccttcggaaaaagagttggtagctcttgat |
| (human variant | | ccggcaaacaaaccaccgctggtagcggtggtttttttgtttgcaagcagc |
| IL-18) is | | agattacgcgcagaaaaaaaggatctcaagaagatcctttgatcttttcta |
| bolded. The | | cggggtctgacgctcagtggaacgaaaactcacgttaagggattttggtca |
| GOI can be | | tgagattatcaaaaaggatcttcacctagatccttttaaattaaaaatgaa |
| substituted | | gttttaaatcaatctaaagtatatatgagtaacctgaggctatggcagggc |
| with one or | | ctgccgccccgacgttggctgcgagccctgggccttcacccgaacttgggg |
| more different | | ggtggggtggggaaaaggaagaaacgcgggcgtattggccccaatggggtc |
| GOIs. | | tcggtggggtatcgacagagtgccagccctgggaccgaaccccgcgtttat |
| | | gaacaaacgacccaacaccgtgcgttttattctgtctttttattgccgtca |
| | | tagcgcgggttccttccggtattgtctccttccgtgtttcagttagcctcc |
| | | ccctagggtgggcgaagaactccagcatgagatccccgcgctggaggatca |
| | | tccagccggcgtcccggaaaacgattccgaagcccaacctttcatagaagg |
| | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggacatgagagtcccagctc |
| | | aactgcttggtcttcttctgctgtggttgcgcggtgccagatgtcacttcg |
| | | ggaagttggagtccaagttgagtgttattcgcaacctcaatgatcaggtgt |
| | | tgttcatagaccagggtaaccgcccactttttgaagatatgaccgatagcg |
| | | attgccgcgacaacgcccccaggaccatcttcataataagtgcgtacggag |
| | | actcacgggcccgggggaaggctgttacaatatccgtaaagtgcgagaaga |
| | | tctccacgctttcttgcgaaaataagattatcagttttaaagaaatgaacc |
| | | ctcctgacaacataaaagatacgaaatcagacatcatcttcttccagcgct |
| | | cagttcctggacatgataataagatgcagttcgagtcatctagctatgaag |
| | | ggtattttctggcttgtgagaaagaacgggatttgtttaaacttatactga |
| | | agaaggaggatgaacttggtgatcggtctattatgtttaccgtacaaaatg |
| | | aggactagcatcgatgatatcgcggccgcatacagcagcaattggcaagct |
| | | gcttacatagaactcgcggcgattggcatgccgccttaaaatttttatttt |
| | | atttttcttttcttttccgaatcggattttgtttttaatatttcaaaaaaa |
| | | aaaaaaaaa |
| Nucleic Acid | 94 | aaaaaaaaaacgcggaagagccgcgtgataacgcaggaaagaacatgtgag |
| Template, | | caaaaggccagcaaaaggccaggaaccgtaaaaaggccgcgttgctggcgt |
| sam002-mouse | | ttttccataggctccgcccccctgacgagcatcacaaaaatcgacgctcaa |
| IL-12-IRES- | | gtcagaggtggcgaaacccgacaggactataaagataccaggcgtttcccc |
| mouse mutant | | ctggaagctccctcgtgcgctctcctgttccgaccctgccgcttaccggat |
| IL18, kana | | acctgtccgcctttctcccttcgggaagcgtggcgctttctcatagctcac |
| resistance | | gctgtaggtatctcagttcggtgtaggtcgttcgctccaagctgggctgtg |
| (SVP-525)- | | tgcacgaaccccccgttcagcccgaccgctgcgccttatccggtaactatc |
| the GOI | | gtcttgagtccaacccggtaagacacgacttatcgccactggcagcagcca |
| (mouse IL-12) | | ctggtaacaggattagcagagcgaggtatgtaggcggtgctacagagttct |
| is bolded; the | | tgaagtggtggcctaactacggctacactagaagaacagtatttggtatct |
| GOI (mouse | | gcgctctgctgaagccagttaccttcggaaaaagagttggtagctcttgat |
| variant IL-18) | | ccggcaaacaaaccaccgctggtagcggtggtttttttgtttgcaagcagc |
| is italicized and | | agattacgcgcagaaaaaaaggatctcaagaagatcctttgatcttttcta |
| underlined; | | cggggtctgacgctcagtggaacgaaaactcacgttaagggattttggtca |
| and the IRES is | | tgagattatcaaaaaggatcttcacctagatccttttaaattaaaaatgaa |
| bolded and | | gttttaaatcaatctaaagtatatatgagtaacctgaggctatggcagggc |
| italicized. The | | ctgccgccccgacgttggctgcgagccctgggccttcacccgaacttgggg |
| GOI can be | | ggtggggtggggaaaaggaagaaacgcgggcgtattggccccaatggggtc |
| substituted | | tcggtggggtatcgacagagtgccagccctgggaccgaaccccgcgtttat |
| with one or | | gaacaaacgacccaacaccgtgcgttttattctgtctttttattgccgtca |
| more different | | tagcgcgggttccttccggtattgtctccttccgtgtttcagttagcctcc |
| GOIs and the | | ccctagggtgggcgaagaactccagcatgagatccccgcgctggaggatca |
| IRES is | | tccagccggcgtcccggaaaacgattccgaagcccaacctttcatagaagg |
| removed. | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggactggacctggcgaatac |
| | | tgttcttggttgccgccgctacagggactcacgcaatgtgggagcttgaaa |
| | | aagacgtctatgtagtagaagtggactggacacctgatgctcctggcgaga |
| | | cagttaacctcacatgcgatacccctgaggaagatgatatcacctggactt |
| | | ctgaccagagacacggggtgattgggagcggcaaaaccctgacgatcactg |
| | | tgaaggagtttctggacgccggccagtatacctgtcacaaggggggggaga |
| | | ccctgagtcatagccacctgttgctgcacaagaaggagaatggcatctggt |
| | | ctacagagatcctgaagaactttaagaacaagaccttcctgaagtgtgaag |
| | | caccaaactacagtggtcgctttacctgcagctggctggtccaaagaaaca |
| | | tggacctgaaatttaatataaagagtagttcctcgagtcctgattccaggg |
| | | ccgtgacgtgcggcatggcaagcctttcagccgaaaaagtcacgctggatc |
| | | agcgagactatgagaagtacagcgttagctgtcaggaggacgtaacttgcc |
| | | cgactgccgaggagactctgcccatagagctcgctctggaggccaggcagc |
| | | agaacaaatatgagaattacagcactagtttctttattagagacatcatca |
| | | aacccgacccacccaagaatctgcagatgaagccgctgaagaatagtcagg |
| | | tcgaggtttcctgggaatatccagattcatggtccactccgcattcttatt |
| | | tttccttaaaattctttgttaggattcagcggaaaaaagaaaagatgaaag |
| | | agacggaggaagggtgcaaccagaagggggccttcctggtggaaaagacaa |
| | | gcactgaggtccaatgtaagggtgggaacgtttgcgtgcaggctcaggatc |
| | | gctactacaacagcagttgctctaagtgggcctgcgtaccttgtcgcgtca |
| | | ggagtggaggggggtcagggggtggctcaggcggcggcagtgggggcagca |
| | | gggtgatcccagtgtctgggccggcccgttgcttgtctcaatccagaaacc |
| | | tcctcaagaccactgacgatatggtaaagactgcccgagagaagctaaaac |
| | | actactcttgtacagctgaagatatagaccatgaggatataacacgggacc |
| | | agacctctactctgaaaacctgtctgcctcttgagctgcacaagaacgagt |
| | | cctgtctggctacccgcgaaacctcaagcacaaccagaggtagttgcctgc |
| | | ccccacaaaagacatcgcttatgatgaccttgtgtctgggatctatttatg |
| | | aggacctgaagatgtaccaaactgagttccaggcaataaatgctgctctcc |
| | | agaatcacaatcatcaacaaatcatccttgataaggggatgctggtcgcaa |
| | | tcgacgagctcatgcaatcgctgaaccacaatggggaaaccctcaggcaga |
| | | aaccaccggtgggagaggccgacccctaccgtgttaaaatgaagttgtgta |
| | | ttcttttgcatgcattctctacaagagtcgttaccatcaatcgcgtcatgg |
| | | ggtacctgtcatcagcctaggcgatcgcgctagcaacggtttccctctagc |
| | | gggatcaattccgccccccccccctaacgttactggccgaagccgcttgga |
| | | ataaggccggtgtgcgtttgtctatatgttattttccaccatattgccgtc |
| | | ttttggcaatgtgagggcccggaaacctggccctgtcttcttgacgagcat |
| | | tcctaggggtctttcccctctcgccaaaggaatgcaaggtctgttgaatgt |
| | | cgtgaaggaagcagttcctctggaagcttcttgaagacaaacaacgtctgt |
| | | agcgaccctttgcaggcagcggaaccccccacctggcgacaggtgcctctg |
| | | cggccaaaagccacgtgtataagatacacctgcaaaggcggcacaacccca |
| | | gtgccacgttgtgagttggatagttgtggaaagagtcaaatggctctcctc |
| | | aagcgtattcaacaaggggctgaaggatgcccagaaggtaccccattgtat |
| | | gggatctgatctggggcctcggtgcacatgctttacatgtgtttagtcgag |
| | | gttaaaaaaacgtctaggccccccgaaccacggggacgtggttttcctttg |
| | | aaaaacacgataataacgcgttgccaccatgacctcccggcttgtgagggt |
| | | actggctgctgctatgctggtggctgctgctgtgagtgtggcacacttcgg |
| | | acgactccattgtactacagccgttatacgaaatatcaatgatcaggtgtt |
| | | gtttgtagacaaaaggcagcccgtgtttgaagacatgactgatattgatca |
| | | gagtgcctctgagccccagactagactgattatatatgcttatggcgactc |
| | | tagagctagagggaaagcagtaactctgtcagtgaaagacagcaagatgtc |
| | | taccctgtcttgcaagaataaaataatctcatttgaggagatggacccccc |
| | | tgaaaacatagatgacatccaaagtgacctgatattctttcagaagcgggt |
| | | acccggtcataacaaaatggagttcgagtcaagcctctatgagggtcactt |
| | | tctcgcctgccaaaaggaagatgacgccttcaaattgatcttgaaaaaaaa |
| | | agacgagaatggtgataagtctgtcatgttcacactcactaatttgcatca |
| | | gtcatagcatcgatgatatcgcggccgcatacagcagcaattggcaagctg |
| | | cttacatagaactcgcggcgattggcatgccgccttaaaatttttatttta |
| | | tttttcttttcttttccgaatcggattttgtttttaatatttcaaaaaaaa |
| | | aaaaaaaa |
| Nucleic Acid | 95 | aaaaaaaaaacgcggaagagccgcgtgataacgcaggaaagaacatgtgag |
| Template, | | caaaaggccagcaaaaggccaggaaccgtaaaaaggccgcgttgctggcgt |
| sam002-mouse | | ttttccataggctccgcccccctgacgagcatcacaaaaatcgacgctcaa |
| IL-12-IRES- | | gtcagaggtggcgaaacccgacaggactataaagataccaggcgtttcccc |
| mouse mutant | | ctggaagctccctcgtgcgctctcctgttccgaccctgccgcttaccggat |
| IL18, kana | | acctgtccgcctttctcccttcgggaagcgtggcgctttctcatagctcac |
| resistance | | gctgtaggtatctcagttcggtgtaggtcgttcgctccaagctgggctgtg |
| (SVP-526)- | | tgcacgaaccccccgttcagcccgaccgctgcgccttatccggtaactatc |
| the GOI | | gtcttgagtccaacccggtaagacacgacttatcgccactggcagcagcca |
| (mouse IL-12) | | ctggtaacaggattagcagagcgaggtatgtaggcggtgctacagagttct |
| is bolded; the | | tgaagtggtggcctaactacggctacactagaagaacagtatttggtatct |
| GOI (mouse | | gcgctctgctgaagccagttaccttcggaaaaagagttggtagctcttgat |
| IL-18) is | | ccggcaaacaaaccaccgctggtagcggtggtttttttgtttgcaagcagc |
| italicized and | | agattacgcgcagaaaaaaaggatctcaagaagatcctttgatcttttcta |
| underlined; | | cggggtctgacgctcagtggaacgaaaactcacgttaagggattttggtca |
| and the IRES is | | tgagattatcaaaaaggatcttcacctagatccttttaaattaaaaatgaa |
| bolded and | | gttttaaatcaatctaaagtatatatgagtaacctgaggctatggcagggc |
| italicized. The | | ctgccgccccgacgttggctgcgagccctgggccttcacccgaacttgggg |
| GOI can be | | ggtggggtggggaaaaggaagaaacgcgggcgtattggccccaatggggtc |
| substituted | | tcggtggggtatcgacagagtgccagccctgggaccgaaccccgcgtttat |
| with one or | | gaacaaacgacccaacaccgtgcgttttattctgtctttttattgccgtca |
| more different | | tagcgcgggttccttccggtattgtctccttccgtgtttcagttagcctcc |
| GOIs and the | | ccctagggtgggcgaagaactccagcatgagatccccgcgctggaggatca |
| IRES is | | tccagccggcgtcccggaaaacgattccgaagcccaacctttcatagaagg |
| removed. | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggactggacctggcgaatac |
| | | tgttcttggttgccgccgctacagggactcacgcaatgtgggagcttgaaa |
| | | aagacgtctatgtagtagaagtggactggacacctgatgctcctggcgaga |
| | | cagttaacctcacatgcgatacccctgaggaagatgatatcacctggactt |
| | | ctgaccagagacacggggtgattgggagcggcaaaaccctgacgatcactg |
| | | tgaaggagtttctggacgccggccagtatacctgtcacaaggggggggaga |
| | | ccctgagtcatagccacctgttgctgcacaagaaggagaatggcatctggt |
| | | ctacagagatcctgaagaactttaagaacaagaccttcctgaagtgtgaag |
| | | caccaaactacagtggtcgctttacctgcagctggctggtccaaagaaaca |
| | | tggacctgaaatttaatataaagagtagttcctcgagtcctgattccaggg |
| | | ccgtgacgtgcggcatggcaagcctttcagccgaaaaagtcacgctggatc |
| | | agcgagactatgagaagtacagcgttagctgtcaggaggacgtaacttgcc |
| | | cgactgccgaggagactctgcccatagagctcgctctggaggccaggcagc |
| | | agaacaaatatgagaattacagcactagtttctttattagagacatcatca |
| | | aacccgacccacccaagaatctgcagatgaagccgctgaagaatagtcagg |
| | | tcgaggtttcctgggaatatccagattcatggtccactccgcattcttatt |
| | | tttccttaaaattctttgttaggattcagcggaaaaaagaaaagatgaaag |
| | | agacggaggaagggtgcaaccagaagggggccttcctggtggaaaagacaa |
| | | gcactgaggtccaatgtaagggtgggaacgtttgcgtgcaggctcaggatc |
| | | gctactacaacagcagttgctctaagtgggcctgcgtaccttgtcgcgtca |
| | | ggagtggaggggggtcagggggtggctcaggcggcggcagtgggggcagca |
| | | gggtgatcccagtgtctgggccggcccgttgcttgtctcaatccagaaacc |
| | | tcctcaagaccactgacgatatggtaaagactgcccgagagaagctaaaac |
| | | actactcttgtacagctgaagatatagaccatgaggatataacacgggacc |
| | | agacctctactctgaaaacctgtctgcctcttgagctgcacaagaacgagt |
| | | cctgtctggctacccgcgaaacctcaagcacaaccagaggtagttgcctgc |
| | | ccccacaaaagacatcgcttatgatgaccttgtgtctgggatctatttatg |
| | | aggacctgaagatgtaccaaactgagttccaggcaataaatgctgctctcc |
| | | agaatcacaatcatcaacaaatcatccttgataaggggatgctggtcgcaa |
| | | tcgacgagctcatgcaatcgctgaaccacaatggggaaaccctcaggcaga |
| | | aaccaccggtgggagaggccgacccctaccgtgttaaaatgaagttgtgta |
| | | ttcttttgcatgcattctctacaagagtcgttaccatcaatcgcgtcatgg |
| | | ggtacctgtcatcagcctaggcgatcgcgctagcaacggtttccctctagc |
| | | gggatcaattccgccccccccccctaacgttactggccgaagccgcttgga |
| | | ataaggccggtgtgcgtttgtctatatgttattttccaccatattgccgtc |
| | | ttttggcaatgtgagggcccggaaacctggccctgtcttcttgacgagcat |
| | | tcctaggggtctttcccctctcgccaaaggaatgcaaggtctgttgaatgt |
| | | cgtgaaggaagcagttcctctggaagcttcttgaagacaaacaacgtctgt |
| | | agcgaccctttgcaggcagcggaaccccccacctggcgacaggtgcctctg |
| | | cggccaaaagccacgtgtataagatacacctgcaaaggcggcacaacccca |
| | | gtgccacgttgtgagttggatagttgtggaaagagtcaaatggctctcctc |
| | | aagcgtattcaacaaggggctgaaggatgcccagaaggtaccccattgtat |
| | | gggatctgatctggggcctcggtgcacatgctttacatgtgtttagtcgag |
| | | gttaaaaaaacgtctaggccccccgaaccacggggacgtggttttcctttg |
| | | aaaaacacgataataacgcgttgccaccatgacctcccggcttgtgagggt |
| | | actggctgctgctatgctggtggctgctgctgtgagtgtggcaaactttgg |
| | | gagactccactgtaccactgccgttatacggaatataaacgatcaggtcct |
| | | ctttgtagacaagagacagccagttttcgaggacatgactgatatagatca |
| | | gagtgcctctgagcctcaaactcggctcataatatacatgtataaggatag |
| | | cgaggttcggggacttgccgtgactttgtccgtgaaagactcaaaaatgag |
| | | caccctgtcatgcaaaaataagataatttctttcgaggaaatggaccctcc |
| | | cgagaacattgatgatatccagtccgacttgatatttttccagaaacgggt |
| | | tcccggtcataacaaaatggagtttgagtcttcattgtacgagggtcattt |
| | | tttggcatgccagaaggaggatgatgccttcaaactgattttgaagaaaaa |
| | | agacgagaacggagacaagtctgtcatgtttacacttaccaaccttcatca |
| | | atcctagcatcgatgatatcgcggccgcatacagcagcaattggcaagctg |
| | | cttacatagaactcgcggcgattggcatgccgccttaaaatttttatttta |
| | | tttttcttttcttttccgaatcggattttgtttttaatatttcaaaaaaaa |
| | | aaaaaaaa |
| Nucleic Acid | 96 | aaaaaaaaaacgcggaagagccgcgtgataacgcaggaaagaacatgtgag |
| Template, | | caaaaggccagcaaaaggccaggaaccgtaaaaaggccgcgttgctggcgt |
| sam002-human | | ttttccataggctccgcccccctgacgagcatcacaaaaatcgacgctcaa |
| IL-12-IRES- | | gtcagaggtggcgaaacccgacaggactataaagataccaggcgtttcccc |
| human wild | | ctggaagctccctcgtgcgctctcctgttccgaccctgccgcttaccggat |
| type IL-18, | | acctgtccgcctttctcccttcgggaagcgtggcgctttctcatagctcac |
| kana resistance | | gctgtaggtatctcagttcggtgtaggtcgttcgctccaagctgggctgtg |
| (SVP-640)- | | tgcacgaaccccccgttcagcccgaccgctgcgccttatccggtaactatc |
| the GOI | | gtcttgagtccaacccggtaagacacgacttatcgccactggcagcagcca |
| (human IL-12) | | ctggtaacaggattagcagagcgaggtatgtaggcggtgctacagagttct |
| is bolded; the | | tgaagtggtggcctaactacggctacactagaagaacagtatttggtatct |
| GOI (human | | gcgctctgctgaagccagttaccttcggaaaaagagttggtagctcttgat |
| IL-18) is | | ccggcaaacaaaccaccgctggtagcggtggtttttttgtttgcaagcagc |
| italicized and | | agattacgcgcagaaaaaaaggatctcaagaagatcctttgatcttttcta |
| underlined; | | cggggtctgacgctcagtggaacgaaaactcacgttaagggattttggtca |
| and the IRES is | | tgagattatcaaaaaggatcttcacctagatccttttaaattaaaaatgaa |
| bolded and | | gttttaaatcaatctaaagtatatatgagtaacctgaggctatggcagggc |
| italicized. The | | ctgccgccccgacgttggctgcgagccctgggccttcacccgaacttgggg |
| GOI can be | | ggtggggtggggaaaaggaagaaacgcgggcgtattggccccaatggggtc |
| substituted | | tcggtggggtatcgacagagtgccagccctgggaccgaaccccgcgtttat |
| with one or | | gaacaaacgacccaacaccgtgcgttttattctgtctttttattgccgtca |
| more different | | tagcgcgggttccttccggtattgtctccttccgtgtttcagttagcctcc |
| GOIs and the | | ccctagggtgggcgaagaactccagcatgagatccccgcgctggaggatca |
| IRES is | | tccagccggcgtcccggaaaacgattccgaagcccaacctttcatagaagg |
| removed. | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggactggacctggcgaatac |
| | | tgttcttggttgccgccgctacagggactcacgcaatatgggagctgaaga |
| | | aggacgtgtatgtcgtggagctggactggtacccagatgctcctggcgaaa |
| | | tggtggttttaacatgtgatacccccgaggaggacggcatcacatggactc |
| | | tggaccagagttctgaggtgctggggtccggcaagactctgacaatccagg |
| | | ttaaggagttcggcgacgcaggacagtacacttgtcacaagggaggtgagg |
| | | tgctttctcacagcctgttgctgctccataagaaggaagacggtatttggt |
| | | caaccgacatcctcaaggaccagaaggagcccaaaaacaagacctttctga |
| | | gatgtgaggccaagaattacagcggtagattcacttgttggtggctcacca |
| | | ccatatccacagacttgaccttcagtgtcaaaagttcacgagggagctcag |
| | | atcctcaaggcgttacctgtggcgcagcgacgctgtccgcagaaagagtca |
| | | ggggagacaacaaggaatacgagtactctgtcgagtgccaggaggattccg |
| | | cctgtccggccgcagaggagtctttacctattgaggtgatggtcgatgccg |
| | | tgcacaagcttaagtacgagaattacacatcaagttttttcatccgcgaca |
| | | tcattaaacctgatccaccaaagaacctgcagctcaagcctctgaagaata |
| | | gcaggcaggtcgaggtaagctgggagtatcctgatacctggtccacccccc |
| | | acagttatttcagcctcaccttctgcgtccaagtccagggaaagagcaaga |
| | | gagagaagaaggatagggtgttcacagataagacttcagctactgtgatct |
| | | gcagaaagaatgcgtctatctctgtgcgagcacaagacaggtactacagtt |
| | | ctagctggagcgagtgggcatcagtcccctgcagtggtggcggaagcggag |
| | | ggggcagcggaggtgggagcggagggagcaggaacctcccagttgctacac |
| | | ctgacccgggaatgtttccatgcctccaccattcccagaatctcctccgag |
| | | ccgtgtccaatatgctgcaaaaggctcggcagaccttggagttttaccctt |
| | | gcacctcagaagaaatcgatcatgaggatatcacaaaggataagacgagca |
| | | ctgttgaggcatgcctgcccctggagctaactaagaatgagtcttgcctga |
| | | acagcagggagacttccttcattaccaacggtagctgtcttgccagcagga |
| | | agacatcttttatgatggccctgtgtctatctagcatatatgaagacctga |
| | | agatgtaccaggtggaattcaaaaccatgaatgctaagcttctcatggatc |
| | | ccaagaggcaaatcttcctggaccagaatatgcttgctgtcatagatgaac |
| | | tgatgcaggcgttgaattttaacagcgagacggtgcctcaaaaaagctcac |
| | | tggaagaacctgatttttataaaacgaagatcaagctgtgtattttactac |
| | | acgcctttagaatccgcgctgttaccatcgacagagtaatgtcctacctaa |
| | | atgcttcataggcgatcgcgctagcaacggtttccctctagcgggatcaat |
| | | tccgccccccccccctaacgttactggccgaagccgcttggaataaggccg |
| | | gtgtgcgtttgtctatatgttattttccaccatattgccgtcttttggcaa |
| | | tgtgagggcccggaaacctggccctgtcttcttgacgagcattcctagggg |
| | | tctttcccctctcgccaaaggaatgcaaggtctgttgaatgtcgtgaagga |
| | | agcagttcctctggaagcttcttgaagacaaacaacgtctgtagcgaccct |
| | | ttgcaggcagcggaaccccccacctggcgacaggtgcctctgcggccaaaa |
| | | gccacgtgtataagatacacctgcaaaggcggcacaaccccagtgccacgt |
| | | tgtgagttggatagttgtggaaagagtcaaatggctctcctcaagcgtatt |
| | | caacaaggggctgaaggatgcccagaaggtaccccattgtatgggatctga |
| | | tctggggcctcggtgcacatgctttacatgtgtttagtcgaggttaaaaaa |
| | | acgtctaggccccccgaaccacggggacgtggttttcctttgaaaaacacg |
| | | ataataacgcgttgccaccatggacatgagagtcccagctcaactgcttgg |
| | | tcttcttctgctgtggttgcgcggtgccagatgttactttggaaagcttga |
| | | gtccaaactttctgtcataagaaacttgaacgaccaggtgttgtttataga |
| | | ccagggtaatcgacctctcttcgaggatatgacagatagcgactgcagaga |
| | | taacgccccccgcaccatattcataatatccatgtataaggactcacaacc |
| | | aagaggcatggccgtaacaataagcgtaaaatgtgagaaaatctctaccct |
| | | gagctgtgagaacaagatcataagcttcaaggaaatgaacccaccagacaa |
| | | catcaaggatactaaaagtgacattatcttcttccaacggagcgtcccagg |
| | | ccacgacaacaagatgcagttcgagtccagcagttacgaaggttacttcct |
| | | ggcgtgcgagaaagagcgcgacctttttaagctcattctgaagaaggaaga |
| | | tgagttgggtgacaggagcattatgttcacggtccagaatgaggactagca |
| | | tcgatgatatcgcggccgcatacagcagcaattggcaagctgcttacatag |
| | | aactcgcggcgattggcatgccgccttaaaatttttattttatttttcttt |
| | | tcttttccgaatcggattttgtttttaatatttcaaaaaaaaaaaaaaaa |
| Nucleic Acid | 97 | aaaaaaaaaacgcggaagagccgcgtgataacgcaggaaagaacatgtgag |
| Template, | | caaaaggccagcaaaaggccaggaaccgtaaaaaggccgcgttgctggcgt |
| sam002-human | | ttttccataggctccgcccccctgacgagcatcacaaaaatcgacgctcaa |
| IL-12-IRES- | | gtcagaggtggcgaaacccgacaggactataaagataccaggcgtttcccc |
| human mutant | | ctggaagctccctcgtgcgctctcctgttccgaccctgccgcttaccggat |
| IL-18, kana | | acctgtccgcctttctcccttcgggaagcgtggcgctttctcatagctcac |
| resistance | | gctgtaggtatctcagttcggtgtaggtcgttcgctccaagctgggctgtg |
| (SVP-641)- | | tgcacgaaccccccgttcagcccgaccgctgcgccttatccggtaactatc |
| the GOI | | gtcttgagtccaacccggtaagacacgacttatcgccactggcagcagcca |
| (human IL-12) | | ctggtaacaggattagcagagcgaggtatgtaggcggtgctacagagttct |
| is bolded; the | | tgaagtggtggcctaactacggctacactagaagaacagtatttggtatct |
| GOI (human | | gcgctctgctgaagccagttaccttcggaaaaagagttggtagctcttgat |
| variant IL-18) | | ccggcaaacaaaccaccgctggtagcggtggtttttttgtttgcaagcagc |
| is italicized and | | agattacgcgcagaaaaaaaggatctcaagaagatcctttgatcttttcta |
| underlined; the | | cggggtctgacgctcagtggaacgaaaactcacgttaagggattttggtca |
| IRES is bolded | | tgagattatcaaaaaggatcttcacctagatccttttaaattaaaaatgaa |
| and italicized. | | gttttaaatcaatctaaagtatatatgagtaacctgaggctatggcagggc |
| The GOI can | | ctgccgccccgacgttggctgcgagccctgggccttcacccgaacttgggg |
| be substituted | | ggtggggtggggaaaaggaagaaacgcgggcgtattggccccaatggggtc |
| with one or | | tcggtggggtatcgacagagtgccagccctgggaccgaaccccgcgtttat |
| more different | | gaacaaacgacccaacaccgtgcgttttattctgtctttttattgccgtca |
| GOIs and the | | tagcgcgggttccttccggtattgtctccttccgtgtttcagttagcctcc |
| IRES is | | ccctagggtgggcgaagaactccagcatgagatccccgcgctggaggatca |
| removed. | | tccagccggcgtcccggaaaacgattccgaagcccaacctttcatagaagg |
| | | cggcggtggaatcgaaatctcgtgatggcaggttgggcgtcgcttggtcgg |
| | | tcatttcgaaccccagagtcccgctcagaagaactcgtcaagaaggcgata |
| | | gaaggcgatgcgctgcgaatcgggagcggcgataccgtaaagcacgaggaa |
| | | gcggtcagcccattcgccgccaagctcttcagcaatatcacgggtagccaa |
| | | cgctatgtcctgatagcgatccgccacacccagccggccacagtcgatgaa |
| | | tccagaaaagcggccattttccaccatgatattcggcaagcaggcatcgcc |
| | | atgggtcacgacgagatcctcgccgtcgggcatgctcgccttgagcctggc |
| | | gaacagttcggctggcgcgagcccctgatgctcttcgtccagatcatcctg |
| | | atcgacaagaccggcttccatccgagtacgtgctcgctcgatgcgatgttt |
| | | cgcttggtggtcgaatgggcaggtagccggatcaagcgtatgcagccgccg |
| | | cattgcatcagccatgatggatactttctcggcaggagcaaggtgagatga |
| | | caggagatcctgccccggcacttcgcccaatagcagccagtcccttcccgc |
| | | ttcagtgacaacgtcgagcacagctgcgcaaggaacgcccgtcgtggccag |
| | | ccacgatagccgcgctgcctcgtcttgcagttcattcagggcaccggacag |
| | | gtcggtcttgacaaaaagaaccgggcgcccctgcgctgacagccggaacac |
| | | ggcggcatcagagcagccgattgtctgttgtgcccagtcatagccgaatag |
| | | cctctccacccaagcggccggagaacctgcgtgcaatccatcttgttcaat |
| | | catgcgaaacgatcctcatcctgtctcttgatcgatctttgcaaaagccta |
| | | ggcctccaaaaaagcctcctcactacttctggaatagctcagaggccgagg |
| | | cggcctcggcctctgcataaataaaaaaaattagtcagccatggggcggag |
| | | aatgggcggaactgggcggagttaggggcgggatgggcggagttaggggcg |
| | | ggactatggttgctgactaattgagatgcatgctttgcatacttctgcctg |
| | | ctggggagcctggggactttccacacctggttgctgactaattgagatgca |
| | | tgctttgcatacttctgcctgctggggagcctggggactttccacacccta |
| | | actgacacacattccacagctggttctttccgcctcaggactcttcctttt |
| | | tcaatattattgaagcatttatcagggttattgtctcatgagcggatacat |
| | | atttgaatgtatttagaaaaataaacaaataggggttccgcgcacatttcc |
| | | ccgaaaagtgccacctgagctctaatacgactcactataggataggcggcg |
| | | catgagagaagcccagaccaattacctacccaaaatggagaaagttcacgt |
| | | tgacatcgaggaagacagcccattcctcagagctttgcagcggagcttccc |
| | | gcagtttgaggtagaagccaagcaggtcactgataatgaccatgctaatgc |
| | | cagagcgttttcgcatctggcttcaaaactgatcgaaacggaggtggaccc |
| | | atccgacacgatccttgacattggaagtgcgcccgcccgcagaatgtattc |
| | | taagcacaagtatcattgtatctgtccgatgagatgtgcggaagatccgga |
| | | cagattgtataagtatgcaactaagctgaagaaaaactgtaaggaaataac |
| | | tgataaggaattggacaagaaaatgaaggagctggccgccgtcatgagcga |
| | | ccctgacctggaaactgagactatgtgcctccacgacgacgagtcgtgtcg |
| | | ctacgaagggcaagtcgctgtttaccaggatgtatacgcggttgacggacc |
| | | gacaagtctctatcaccaagccaataagggagttagagtcgcctactggat |
| | | aggctttgacaccaccccttttatgtttaagaacttggctggagcatatcc |
| | | atcatactctaccaactgggccgacgaaaccgtgttaacggctcgtaacat |
| | | aggcctatgcagctctgacgttatggagcggtcacgtagagggatgtccat |
| | | tcttagaaagaagtatttgaaaccatccaacaatgttctattctctgttgg |
| | | ctcgaccatctaccacgagaagagggacttactgaggagctggcacctgcc |
| | | gtctgtatttcacttacgtggcaagcaaaattacacatgtcggtgtgagac |
| | | tatagttagttgcgacgggtacgtcgttaaaagaatagctatcagtccagg |
| | | cctgtatgggaagccttcaggctatgctgctacgatgcaccgcgagggatt |
| | | cttgtgctgcaaagtgacagacacattgaacggggagagggtctcttttcc |
| | | cgtgtgcacgtatgtgccagctacattgtgtgaccaaatgactggcatact |
| | | ggcaacagatgtcagtgcggacgacgcgcaaaaactgctggttgggctcaa |
| | | ccagcgtatagtcgtcaacggtcgcacccagagaaacaccaataccatgaa |
| | | aaattaccttttgcccgtagtggcccaggcatttgctaggtgggcaaagga |
| | | atataaggaagatcaagaagatgaaaggccactaggactacgagatagaca |
| | | gttagtcatggggtgttgttgggcttttagaaggcacaagataacatctat |
| | | ttataagcgcccggatacccaaaccatcatcaaagtgaacagcgatttcca |
| | | ctcattcgtgctgcccaggataggcagtaacacattggagatcgggctgag |
| | | aacaagaatcaggaaaatgttagaggagcacaaggagccgtcacctctcat |
| | | taccgccgaggacgtacaagaagctaagtgcgcagccgatgaggctaagga |
| | | ggtgcgtgaagccgaggagttgcgcgcagctctaccacctttggcagctga |
| | | tgttgaggagcccactctggaagccgatgtcgacttgatgttacaagaggc |
| | | tggggccggctcagtggagacacctcgtggcttgataaaggttaccagcta |
| | | cgatggcgaggacaagatcggctcttacgctgtgctttctccgcaggctgt |
| | | actcaagagtgaaaaattatcttgcatccaccctctcgctgaacaagtcat |
| | | agtgataacacactctggccgaaaagggcgttatgccgtggaaccatacca |
| | | tggtaaagtagtggtgccagagggacatgcaatacccgtccaggactttca |
| | | agctctgagtgaaagtgccaccattgtgtacaacgaacgtgagttcgtaaa |
| | | caggtacctgcaccatattgccacacatggaggagcgctgaacactgatga |
| | | agaatattacaaaactgtcaagcccagcgagcacgacggcgaatacctgta |
| | | cgacatcgacaggaaacagtgcgtcaagaaagaactagtcactgggctagg |
| | | gctcacaggcgagctggtggatcctcccttccatgaattcgcctacgagag |
| | | tctgagaacacgaccagccgctccttaccaagtaccaaccataggggtgta |
| | | tggcgtgccaggatcaggcaagtctggcatcattaaaagcgcagtcaccaa |
| | | aaaagatctagtggtgagcgccaagaaagaaaactgtgcagaaattataag |
| | | ggacgtcaagaaaatgaaagggctggacgtcaatgccagaactgtggactc |
| | | agtgctcttgaatggatgcaaacaccccgtagagaccctgtatattgacga |
| | | agcttttgcttgtcatgcaggtactctcagagcgctcatagccattataag |
| | | acctaaaaaggcagtgctctgcggggatcccaaacagtgcggtttttttaa |
| | | catgatgtgcctgaaagtgcattttaaccacgagatttgcacacaagtctt |
| | | ccacaaaagcatctctcgccgttgcactaaatctgtgacttcggtcgtctc |
| | | aaccttgttttacgacaaaaaaatgagaacgacgaatccgaaagagactaa |
| | | gattgtgattgacactaccggcagtaccaaacctaagcaggacgatctcat |
| | | tctcacttgtttcagagggtgggtgaagcagttgcaaatagattacaaagg |
| | | caacgaaataatgacggcagctgcctctcaagggctgacccgtaaaggtgt |
| | | gtatgccgttcggtacaaggtgaatgaaaatcctctgtacgcacccacctc |
| | | agaacatgtgaacgtcctactgacccgcacggaggaccgcatcgtgtggaa |
| | | aacactagccggcgacccatggataaaaacactgactgccaagtaccctgg |
| | | gaatttcactgccacgatagaggagtggcaagcagagcatgatgccatcat |
| | | gaggcacatcttggagagaccggaccctaccgacgtcttccagaataaggc |
| | | aaacgtgtgttgggccaaggctttagtgccggtgctgaagaccgctggcat |
| | | agacatgaccactgaacaatggaacactgtggattattttgaaacggacaa |
| | | agctcactcagcagagatagtattgaaccaactatgcgtgaggttctttgg |
| | | actcgatctggactccggtctattttctgcacccactgttccgttatccat |
| | | taggaataatcactgggataactccccgtcgcctaacatgtacgggctgaa |
| | | taaagaagtggtccgtcagctctctcgcaggtacccacaactgcctcgggc |
| | | agttgccactggaagagtctatgacatgaacactggtacactgcgcaatta |
| | | tgatccgcgcataaacctagtacctgtaaacagaagactgcctcatgcttt |
| | | agtcctccaccataatgaacacccacagagtgacttttcttcattcgtcag |
| | | caaattgaagggcagaactgtcctggtggtcggggaaaagttgtccgtccc |
| | | aggcaaaatggttgactggttgtcagaccggcctgaggctaccttcagagc |
| | | tcggctggatttaggcatcccaggtgatgtgcccaaatatgacataatatt |
| | | tgttaatgtgaggaccccatataaataccatcactatcagcagtgtgaaga |
| | | ccatgccattaagcttagcatgttgaccaagaaagcttgtctgcatctgaa |
| | | tcccggcggaacctgtgtcagcataggttatggttacgctgacagggccag |
| | | cgaaagcatcattggtgctatagcgcggcagttcaagttttcccgggtatg |
| | | caaaccgaaatcctcacttgaagagacggaagttctgtttgtattcattgg |
| | | gtacgatcgcaaggcccgtacgcacaattcttacaagctttcatcaacctt |
| | | gaccaacatttatacaggttccagactccacgaagccggatgtgcaccctc |
| | | atatcatgtggtgcgaggggatattgccacggccaccgaaggagtgattat |
| | | aaatgctgctaacagcaaaggacaacctggcggaggggtgtgcggagcgct |
| | | gtataagaaattcccggaaagcttcgatttacagccgatcgaagtaggaaa |
| | | agcgcgactggtcaaaggtgcagctaaacatatcattcatgccgtaggacc |
| | | aaacttcaacaaagtttcggaggttgaaggtgacaaacagttggcagaggc |
| | | ttatgagtccatcgctaagattgtcaacgataacaattacaagtcagtagc |
| | | gattccactgttgtccaccggcatcttttccgggaacaaagatcgactaac |
| | | ccaatcattgaaccatttgctgacagctttagacaccactgatgcagatgt |
| | | agccatatactgcagggacaagaaatgggaaatgactctcaaggaagcagt |
| | | ggctaggagagaagcagtggaggagatatgcatatccgacgactcttcagt |
| | | gacagaacctgatgcagagctggtgagggtgcatccgaagagttctttggc |
| | | tggaaggaagggctacagcacaagcgatggcaaaactttctcatatttgga |
| | | agggaccaagtttcaccaggcggccaaggatatagcagaaattaatgccat |
| | | gtggcccgttgcaacggaggccaatgagcaggtatgcatgtatatcctcgg |
| | | agaaagcatgagcagtattaggtcgaaatgccccgtcgaagagtcggaagc |
| | | ctccacaccacctagcacgctgccttgcttgtgcatccatgccatgactcc |
| | | agaaagagtacagcgcctaaaagcctcacgtccagaacaaattactgtgtg |
| | | ctcatcctttccattgccgaagtatagaatcactggtgtgcagaagatcca |
| | | atgctcccagcctatattgttctcaccgaaagtgcctgcgtatattcatcc |
| | | aaggaagtatctcgtggaaacaccaccggtagacgagactccggagccatc |
| | | ggcagagaaccaatccacagaggggacacctgaacaaccaccacttataac |
| | | cgaggatgagaccaggactagaacgcctgagccgatcatcatcgaagagga |
| | | agaagaggatagcataagtttgctgtcagatggcccgacccaccaggtgct |
| | | gcaagtcgaggcagacattcacgggccgccctctgtatctagctcatcctg |
| | | gtccattcctcatgcatccgactttgatgtggacagtttatccatacttga |
| | | caccctggagggagctagcgtgaccagcggggcaacgtcagccgagactaa |
| | | ctcttacttcgcaaagagtatggagtttctggcgcgaccggtgcctgcgcc |
| | | tcgaacagtattcaggaaccctccacatcccgctccgcgcacaagaacacc |
| | | gtcacttgcacccagcagggcctgctcgagaaccagcctagtttccacccc |
| | | gccaggcgtgaatagggtgatcactagagaggagctcgaggcgcttacccc |
| | | gtcacgcactcctagcaggtcggtctcgagaaccagcctggtctccaaccc |
| | | gccaggcgtaaatagggtgattacaagagaggagtttgaggcgttcgtagc |
| | | acaacaacaatgacggtttgatgcgggtgcatacatcttttcctccgacac |
| | | cggtcaagggcatttacaacaaaaatcagtaaggcaaacggtgctatccga |
| | | agtggtgttggagaggaccgaattggagatttcgtatgccccgcgcctcga |
| | | ccaagaaaaagaagaattactacgcaagaaattacagttaaatcccacacc |
| | | tgctaacagaagcagataccagtccaggaaggtggagaacatgaaagccat |
| | | aacagctagacgtattctgcaaggcctagggcattatttgaaggcagaagg |
| | | aaaagtggagtgctaccgaaccctgcatcctgttcctttgtattcatctag |
| | | tgtgaaccgtgccttttcaagccccaaggtcgcagtggaagcctgtaacgc |
| | | catgttgaaagagaactttccgactgtggcttcttactgtattattccaga |
| | | gtacgatgcctatttggacatggttgacggagcttcatgctgcttagacac |
| | | tgccagtttttgccctgcaaagctgcgcagctttccaaagaaacactccta |
| | | tttggaacccacaatacgatcggcagtgccttcagcgatccagaacacgct |
| | | ccagaacgtcctggcagctgccacaaaaagaaattgcaatgtcacgcaaat |
| | | gagagaattgcccgtattggattcggcggcctttaatgtggaatgcttcaa |
| | | gaaatatgcgtgtaataatgaatattgggaaacgtttaaagaaaaccccat |
| | | caggcttactgaagaaaacgtggtaaattacattaccaaattaaaaggacc |
| | | aaaagctgctgctctttttgcgaagacacataatttgaatatgttgcagga |
| | | cataccaatggacaggtttgtaatggacttaaagagagacgtgaaagtgac |
| | | tccaggaacaaaacatactgaagaacggcccaaggtacaggtgatccaggc |
| | | tgccgatccgctagcaacagcgtatctgtgcggaatccaccgagagctggt |
| | | taggagattaaatgcggtcctgcttccgaacattcatacactgtttgatat |
| | | gtcggctgaagactttgacgctattatagccgagcacttccagcctgggga |
| | | ttgtgttctggaaactgacatcgcgtcgtttgataaaagtgaggacgacgc |
| | | catggctctgaccgcgttaatgattctggaagacttaggtgtggacgcaga |
| | | gctgttgacgctgattgaggcggctttcggcgaaatttcatcaatacattt |
| | | gcccactaaaactaaatttaaattcggagccatgatgaaatctggaatgtt |
| | | cctcacactgtttgtgaacacagtcattaacattgtaatcgcaagcagagt |
| | | gttgagagaacggctaaccggatcaccatgtgcagcattcattggagatga |
| | | caatatcgtgaaaggagtcaaatcggacaaattaatggcagacaggtgcgc |
| | | cacctggttgaatatggaagtcaagattatagatgctgtggtgggcgagaa |
| | | agcgccttatttctgtggagggtttattttgtgtgactccgtgaccggcac |
| | | agcgtgccgtgtggcagaccccctaaaaaggctgtttaagcttggcaaacc |
| | | tctggcagcagacgatgaacatgatgatgacaggagaagggcattgcatga |
| | | agagtcaacacgctggaaccgagtgggtattctttcagagctgtgcaaggc |
| | | agtagaatcaaggtatgaaaccgtaggaacttccatcatagttatggccat |
| | | gactactctagctagcagtgttaaatcattcagctacctgagaggggcccc |
| | | tataactctctacggctaacctgaatggactacgacatagtctagtccgcc |
| | | aagttcgaaggcgcgcctctagagccaccatggactggacctggcgaatac |
| | | tgttcttggttgccgccgctacagggactcacgcaatatgggagctgaaga |
| | | aggacgtgtatgtcgtggagctggactggtacccagatgctcctggcgaaa |
| | | tggtggttttaacatgtgatacccccgaggaggacggcatcacatggactc |
| | | tggaccagagttctgaggtgctggggtccggcaagactctgacaatccagg |
| | | ttaaggagttcggcgacgcaggacagtacacttgtcacaagggaggtgagg |
| | | tgctttctcacagcctgttgctgctccataagaaggaagacggtatttggt |
| | | caaccgacatcctcaaggaccagaaggagcccaaaaacaagacctttctga |
| | | gatgtgaggccaagaattacagcggtagattcacttgttggtggctcacca |
| | | ccatatccacagacttgaccttcagtgtcaaaagttcacgagggagctcag |
| | | atcctcaaggcgttacctgtggcgcagcgacgctgtccgcagaaagagtca |
| | | ggggagacaacaaggaatacgagtactctgtcgagtgccaggaggattccg |
| | | cctgtccggccgcagaggagtctttacctattgaggtgatggtcgatgccg |
| | | tgcacaagcttaagtacgagaattacacatcaagttttttcatccgcgaca |
| | | tcattaaacctgatccaccaaagaacctgcagctcaagcctctgaagaata |
| | | gcaggcaggtcgaggtaagctgggagtatcctgatacctggtccacccccc |
| | | acagttatttcagcctcaccttctgcgtccaagtccagggaaagagcaaga |
| | | gagagaagaaggatagggtgttcacagataagacttcagctactgtgatct |
| | | gcagaaagaatgcgtctatctctgtgcgagcacaagacaggtactacagtt |
| | | ctagctggagcgagtgggcatcagtcccctgcagtggtggcggaagcggag |
| | | ggggcagcggaggtgggagcggagggagcaggaacctcccagttgctacac |
| | | ctgacccgggaatgtttccatgcctccaccattcccagaatctcctccgag |
| | | ccgtgtccaatatgctgcaaaaggctcggcagaccttggagttttaccctt |
| | | gcacctcagaagaaatcgatcatgaggatatcacaaaggataagacgagca |
| | | ctgttgaggcatgcctgcccctggagctaactaagaatgagtcttgcctga |
| | | acagcagggagacttccttcattaccaacggtagctgtcttgccagcagga |
| | | agacatcttttatgatggccctgtgtctatctagcatatatgaagacctga |
| | | agatgtaccaggtggaattcaaaaccatgaatgctaagcttctcatggatc |
| | | ccaagaggcaaatcttcctggaccagaatatgcttgctgtcatagatgaac |
| | | tgatgcaggcgttgaattttaacagcgagacggtgcctcaaaaaagctcac |
| | | tggaagaacctgatttttataaaacgaagatcaagctgtgtattttactac |
| | | acgcctttagaatccgcgctgttaccatcgacagagtaatgtcctacctaa |
| | | atgcttcataggcgatcgcgctagcaacggtttccctctagcgggatcaat |
| | | tccgccccccccccctaacgttactggccgaagccgcttggaataaggccg |
| | | gtgtgcgtttgtctatatgttattttccaccatattgccgtcttttggcaa |
| | | tgtgagggcccggaaacctggccctgtcttcttgacgagcattcctagggg |
| | | tctttcccctctcgccaaaggaatgcaaggtctgttgaatgtcgtgaagga |
| | | agcagttcctctggaagcttcttgaagacaaacaacgtctgtagcgaccct |
| | | ttgcaggcagcggaaccccccacctggcgacaggtgcctctgcggccaaaa |
| | | gccacgtgtataagatacacctgcaaaggcggcacaaccccagtgccacgt |
| | | tgtgagttggatagttgtggaaagagtcaaatggctctcctcaagcgtatt |
| | | caacaaggggctgaaggatgcccagaaggtaccccattgtatgggatctga |
| | | tctggggcctcggtgcacatgctttacatgtgtttagtcgaggttaaaaaa |
| | | acgtctaggccccccgaaccacggggacgtggttttcctttgaaaaacacg |
| | | ataataacgcgttgccaccatggacatgagagtcccagctcaactgcttgg |
| | | tcttcttctgctgtggttgcgcggtgccagatgtcacttcgggaagttgga |
| | | gtccaagttgagtgttattcgcaacctcaatgatcaggtgttgttcataga |
| | | ccagggtaaccgcccactttttgaagatatgaccgatagcgattgccgcga |
| | | caacgcccccaggaccatcttcataataagtgcgtacggagactcacgggc |
| | | ccgggggaaggctgttacaatatccgtaaagtgcgagaagatctccacgct |
| | | ttcttgcgaaaataagattatcagttttaaagaaatgaaccctcctgacaa |
| | | cataaaagatacgaaatcagacatcatcttcttccagcgctcagttcctgg |
| | | acatgataataagatgcagttcgagtcatctagctatgaagggtattttct |
| | | ggcttgtgagaaagaacgggatttgtttaaacttatactgaagaaggagga |
| | | tgaacttggtgatcggtctattatgtttaccgtacaaaatgaggactagca |
| | | tcgatgatatcgcggccgcatacagcagcaattggcaagctgcttacatag |
| | | aactcgcggcgattggcatgccgccttaaaatttttattttatttttcttt |
| | | tcttttccgaatcggattttgtttttaatatttcaaaaaaaaaaaaaaaa |
| Nucleic Acid | 98 | aaaaaaaaaacgcgtgataacgcaggaaagaacatgtgagcaaaaggccag |
| Template, | | caaaaggccaggaaccgtaaaaaggccgcgttgctggcgtttttccatagg |
| sam002- | | ctccgcccccctgacgagcatcacaaaaatcgacgctcaagtcagaggtgg |
| Luciferase- | | cgaaacccgacaggactataaagataccaggcgtttccccctggaagctcc |
| Amp resistance | | ctcgtgcgctctcctgttccgaccctgccgcttaccggatacctgtccgcc |
| (SVP-062)- | | tttctcccttcgggaagcgtggcgctttctcatagctcacgctgtaggtat |
| the GOI | | ctcagttcggtgtaggtcgttcgctccaagctgggctgtgtgcacgaaccc |
| (Luciferase, | | cccgttcagcccgaccgctgcgccttatccggtaactatcgtcttgagtcc |
| SEQ ID NO: | | aacccggtaagacacgacttatcgccactggcagcagccactggtaacagg |
| 25) is bolded. | | attagcagagcgaggtatgtaggcggtgctacagagttcttgaagtggtgg |
| The GOI can | | cctaactacggctacactagaagaacagtatttggtatctgcgctctgctg |
| be substituted | | aagccagttaccttcggaaaaagagttggtagctcttgatccggcaaacaa |
| with one or | | accaccgctggtagcggtttttttgtttgcaagcagcagattacgcgcaga |
| more different | | aaaaaaggatctcaagaagatcctttgatcttttctacggggtctgacgct |
| GOIs. | | cagtggaacgaaaactcacgttaagggattttggtcatgagattatcaaaa |
| | | aggatcttcacctagatccttttaaattaaaaatgaagttttaaatcaatc |
| | | taaagtatatatgagtaaacttggtctgacagttaccaatgcttaatcagt |
| | | gaggcacctatctcagcgatctgtctatttcgttcatccatagttgcctga |
| | | ctccccgtcgtgtagataactacgatacgggagggcttaccatctggcccc |
| | | agtgctgcaatgataccgcgagacccacgctcaccggctccagatttatca |
| | | gcaataaaccagccagccggaagggccgagcgcagaagtggtcctgcaact |
| | | ttatccgcctccatccagtctattaattgttgccgggaagctagagtaagt |
| | | agttcgccagttaatagtttgcgcaacgttgttgccattgctacaggcatc |
| | | gtggtgtcacgctcgtcgtttggtatggcttcattcagctccggttcccaa |
| | | cgatcaaggcgagttacatgatcccccatgttgtgcaaaaaagcggttagc |
| | | tccttcggtcctccgatcgttgtcagaagtaagttggccgcagtgttatca |
| | | ctcatggttatggcagcactgcataattctcttactgtcatgccatccgta |
| | | agatgcttttctgtgactggtgagtactcaaccaagtcattctgagaatag |
| | | tgtatgcggcgaccgagttgctcttgcccggcgtcaatacgggataatacc |
| | | gcgccacatagcagaactttaaaagtgctcatcattggaaaacgttcttcg |
| | | gggcgaaaactctcaaggatcttaccgctgttgagatccagttcgatgtaa |
| | | cccactcgtgcacccaactgatcttcagcatcttttactttcaccagcgtt |
| | | tctgggtgagcaaaaacaggaaggcaaaatgccgcaaaaaagggaataagg |
| | | gcgacacggaaatgttgaatactcatactcttcctttttcaatattattga |
| | | agcatttatcagggttattgtctcatgagcggatacatatttgaatgtatt |
| | | tagaaaaataaacaaataggggttccgcgcacatttccccgaaaagtgcca |
| | | cctgagctctaatacgactcactataggataggcggcgcatgagagaagcc |
| | | cagaccaattacctacccaaaatggagaaagttcacgttgacatcgaggaa |
| | | gacagcccattcctcagagctttgcagcggagcttcccgcagtttgaggta |
| | | gaagccaagcaggtcactgataatgaccatgctaatgccagagcgttttcg |
| | | catctggcttcaaaactgatcgaaacggaggtggacccatccgacacgatc |
| | | cttgacattggaagtgcgcccgcccgcagaatgtattctaagcacaagtat |
| | | cattgtatctgtccgatgagatgtgcggaagatccggacagattgtataag |
| | | tatgcaactaagctgaagaaaaactgtaaggaaataactgataaggaattg |
| | | gacaagaaaatgaaggagctggccgccgtcatgagcgaccctgacctggaa |
| | | actgagactatgtgcctccacgacgacgagtcgtgtcgctacgaagggcaa |
| | | gtcgctgtttaccaggatgtatacgcggttgacggaccgacaagtctctat |
| | | caccaagccaataagggagttagagtcgcctactggataggctttgacacc |
| | | accccttttatgtttaagaacttggctggagcatatccatcatactctacc |
| | | aactgggccgacgaaaccgtgttaacggctcgtaacataggcctatgcagc |
| | | tctgacgttatggagcggtcacgtagagggatgtccattcttagaaagaag |
| | | tatttgaaaccatccaacaatgttctattctctgttggctcgaccatctac |
| | | cacgagaagagggacttactgaggagctggcacctgccgtctgtatttcac |
| | | ttacgtggcaagcaaaattacacatgtcggtgtgagactatagttagttgc |
| | | gacgggtacgtcgttaaaagaatagctatcagtccaggcctgtatgggaag |
| | | ccttcaggctatgctgctacgatgcaccgcgagggattcttgtgctgcaaa |
| | | gtgacagacacattgaacggggagagggtctcttttcccgtgtgcacgtat |
| | | gtgccagctacattgtgtgaccaaatgactggcatactggcaacagatgtc |
| | | agtgcggacgacgcgcaaaaactgctggttgggctcaaccagcgtatagtc |
| | | gtcaacggtcgcacccagagaaacaccaataccatgaaaaattaccttttg |
| | | cccgtagtggcccaggcatttgctaggtgggcaaaggaatataaggaagat |
| | | caagaagatgaaaggccactaggactacgagatagacagttagtcatgggg |
| | | tgttgttgggcttttagaaggcacaagataacatctatttataagcgcccg |
| | | gatacccaaaccatcatcaaagtgaacagcgatttccactcattcgtgctg |
| | | cccaggataggcagtaacacattggagatcgggctgagaacaagaatcagg |
| | | aaaatgttagaggagcacaaggagccgtcacctctcattaccgccgaggac |
| | | gtacaagaagctaagtgcgcagccgatgaggctaaggaggtgcgtgaagcc |
| | | gaggagttgcgcgcagctctaccacctttggcagctgatgttgaggagccc |
| | | actctggaagccgatgtcgacttgatgttacaagaggctggggccggctca |
| | | gtggagacacctcgtggcttgataaaggttaccagctacgatggcgaggac |
| | | aagatcggctcttacgctgtgctttctccgcaggctgtactcaagagtgaa |
| | | aaattatcttgcatccaccctctcgctgaacaagtcatagtgataacacac |
| | | tctggccgaaaagggcgttatgccgtggaaccataccatggtaaagtagtg |
| | | gtgccagagggacatgcaatacccgtccaggactttcaagctctgagtgaa |
| | | agtgccaccattgtgtacaacgaacgtgagttcgtaaacaggtacctgcac |
| | | catattgccacacatggaggagcgctgaacactgatgaagaatattacaaa |
| | | actgtcaagcccagcgagcacgacggcgaatacctgtacgacatcgacagg |
| | | aaacagtgcgtcaagaaagaactagtcactgggctagggctcacaggcgag |
| | | ctggtggatcctcccttccatgaattcgcctacgagagtctgagaacacga |
| | | ccagccgctccttaccaagtaccaaccataggggtgtatggcgtgccagga |
| | | tcaggcaagtctggcatcattaaaagcgcagtcaccaaaaaagatctagtg |
| | | gtgagcgccaagaaagaaaactgtgcagaaattataagggacgtcaagaaa |
| | | atgaaagggctggacgtcaatgccagaactgtggactcagtgctcttgaat |
| | | ggatgcaaacaccccgtagagaccctgtatattgacgaagcttttgcttgt |
| | | catgcaggtactctcagagcgctcatagccattataagacctaaaaaggca |
| | | gtgctctgcggggatcccaaacagtgcggtttttttaacatgatgtgcctg |
| | | aaagtgcattttaaccacgagatttgcacacaagtcttccacaaaagcatc |
| | | tctcgccgttgcactaaatctgtgacttcggtcgtctcaaccttgttttac |
| | | gacaaaaaaatgagaacgacgaatccgaaagagactaagattgtgattgac |
| | | actaccggcagtaccaaacctaagcaggacgatctcattctcacttgtttc |
| | | agagggtgggtgaagcagttgcaaatagattacaaaggcaacgaaataatg |
| | | acggcagctgcctctcaagggctgacccgtaaaggtgtgtatgccgttcgg |
| | | tacaaggtgaatgaaaatcctctgtacgcacccacctcagaacatgtgaac |
| | | gtcctactgacccgcacggaggaccgcatcgtgtggaaaacactagccggc |
| | | gacccatggataaaaacactgactgccaagtaccctgggaatttcactgcc |
| | | acgatagaggagtggcaagcagagcatgatgccatcatgaggcacatcttg |
| | | gagagaccggaccctaccgacgtcttccagaataaggcaaacgtgtgttgg |
| | | gccaaggctttagtgccggtgctgaagaccgctggcatagacatgaccact |
| | | gaacaatggaacactgtggattattttgaaacggacaaagctcactcagca |
| | | gagatagtattgaaccaactatgcgtgaggttctttggactcgatctggac |
| | | tccggtctattttctgcacccactgttccgttatccattaggaataatcac |
| | | tgggataactccccgtcgcctaacatgtacgggctgaataaagaagtggtc |
| | | cgtcagctctctcgcaggtacccacaactgcctcgggcagttgccactgga |
| | | agagtctatgacatgaacactggtacactgcgcaattatgatccgcgcata |
| | | aacctagtacctgtaaacagaagactgcctcatgctttagtcctccaccat |
| | | aatgaacacccacagagtgacttttcttcattcgtcagcaaattgaagggc |
| | | agaactgtcctggtggtcggggaaaagttgtccgtcccaggcaaaatggtt |
| | | gactggttgtcagaccggcctgaggctaccttcagagctcggctggattta |
| | | ggcatcccaggtgatgtgcccaaatatgacataatatttgttaatgtgagg |
| | | accccatataaataccatcactatcagcagtgtgaagaccatgccattaag |
| | | cttagcatgttgaccaagaaagcttgtctgcatctgaatcccggcggaacc |
| | | tgtgtcagcataggttatggttacgctgacagggccagcgaaagcatcatt |
| | | ggtgctatagcgcggcagttcaagttttcccgggtatgcaaaccgaaatcc |
| | | tcacttgaagagacggaagttctgtttgtattcattgggtacgatcgcaag |
| | | gcccgtacgcacaattcttacaagctttcatcaaccttgaccaacatttat |
| | | acaggttccagactccacgaagccggatgtgcaccctcatatcatgtggtg |
| | | cgaggggatattgccacggccaccgaaggagtgattataaatgctgctaac |
| | | agcaaaggacaacctggcggaggggtgtgcggagcgctgtataagaaattc |
| | | ccggaaagcttcgatttacagccgatcgaagtaggaaaagcgcgactggtc |
| | | aaaggtgcagctaaacatatcattcatgccgtaggaccaaacttcaacaaa |
| | | gtttcggaggttgaaggtgacaaacagttggcagaggcttatgagtccatc |
| | | gctaagattgtcaacgataacaattacaagtcagtagcgattccactgttg |
| | | tccaccggcatcttttccgggaacaaagatcgactaacccaatcattgaac |
| | | catttgctgacagctttagacaccactgatgcagatgtagccatatactgc |
| | | agggacaagaaatgggaaatgactctcaaggaagcagtggctaggagagaa |
| | | gcagtggaggagatatgcatatccgacgactcttcagtgacagaacctgat |
| | | gcagagctggtgagggtgcatccgaagagttctttggctggaaggaagggc |
| | | tacagcacaagcgatggcaaaactttctcatatttggaagggaccaagttt |
| | | caccaggcggccaaggatatagcagaaattaatgccatgtggcccgttgca |
| | | acggaggccaatgagcaggtatgcatgtatatcctcggagaaagcatgagc |
| | | agtattaggtcgaaatgccccgtcgaagagtcggaagcctccacaccacct |
| | | agcacgctgccttgcttgtgcatccatgccatgactccagaaagagtacag |
| | | cgcctaaaagcctcacgtccagaacaaattactgtgtgctcatcctttcca |
| | | ttgccgaagtatagaatcactggtgtgcagaagatccaatgctcccagcct |
| | | atattgttctcaccgaaagtgcctgcgtatattcatccaaggaagtatctc |
| | | gtggaaacaccaccggtagacgagactccggagccatcggcagagaaccaa |
| | | tccacagaggggacacctgaacaaccaccacttataaccgaggatgagacc |
| | | aggactagaacgcctgagccgatcatcatcgaagaggaagaagaggatagc |
| | | ataagtttgctgtcagatggcccgacccaccaggtgctgcaagtcgaggca |
| | | gacattcacgggccgccctctgtatctagctcatcctggtccattcctcat |
| | | gcatccgactttgatgtggacagtttatccatacttgacaccctggaggga |
| | | gctagcgtgaccagcggggcaacgtcagccgagactaactcttacttcgca |
| | | aagagtatggagtttctggcgcgaccggtgcctgcgcctcgaacagtattc |
| | | aggaaccctccacatcccgctccgcgcacaagaacaccgtcacttgcaccc |
| | | agcagggcctgctcgagaaccagcctagtttccaccccgccaggcgtgaat |
| | | agggtgatcactagagaggagctcgaggcgcttaccccgtcacgcactcct |
| | | agcaggtcggtctcgagaaccagcctggtctccaacccgccaggcgtaaat |
| | | agggtgattacaagagaggagtttgaggcgttcgtagcacaacaacaatga |
| | | cggtttgatgcgggtgcatacatcttttcctccgacaccggtcaagggcat |
| | | ttacaacaaaaatcagtaaggcaaacggtgctatccgaagtggtgttggag |
| | | aggaccgaattggagatttcgtatgccccgcgcctcgaccaagaaaaagaa |
| | | gaattactacgcaagaaattacagttaaatcccacacctgctaacagaagc |
| | | agataccagtccaggaaggtggagaacatgaaagccataacagctagacgt |
| | | attctgcaaggcctagggcattatttgaaggcagaaggaaaagtggagtgc |
| | | taccgaaccctgcatcctgttcctttgtattcatctagtgtgaaccgtgcc |
| | | ttttcaagccccaaggtcgcagtggaagcctgtaacgccatgttgaaagag |
| | | aactttccgactgtggcttcttactgtattattccagagtacgatgcctat |
| | | ttggacatggttgacggagcttcatgctgcttagacactgccagtttttgc |
| | | cctgcaaagctgcgcagctttccaaagaaacactcctatttggaacccaca |
| | | atacgatcggcagtgccttcagcgatccagaacacgctccagaacgtcctg |
| | | gcagctgccacaaaaagaaattgcaatgtcacgcaaatgagagaattgccc |
| | | gtattggattcggcggcctttaatgtggaatgcttcaagaaatatgcgtgt |
| | | aataatgaatattgggaaacgtttaaagaaaaccccatcaggcttactgaa |
| | | gaaaacgtggtaaattacattaccaaattaaaaggaccaaaagctgctgct |
| | | ctttttgcgaagacacataatttgaatatgttgcaggacataccaatggac |
| | | aggtttgtaatggacttaaagagagacgtgaaagtgactccaggaacaaaa |
| | | catactgaagaacggcccaaggtacaggtgatccaggctgccgatccgcta |
| | | gcaacagcgtatctgtgcggaatccaccgagagctggttaggagattaaat |
| | | gcggtcctgcttccgaacattcatacactgtttgatatgtcggctgaagac |
| | | tttgacgctattatagccgagcacttccagcctggggattgtgttctggaa |
| | | actgacatcgcgtcgtttgataaaagtgaggacgacgccatggctctgacc |
| | | gcgttaatgattctggaagacttaggtgtggacgcagagctgttgacgctg |
| | | attgaggcggctttcggcgaaatttcatcaatacatttgcccactaaaact |
| | | aaatttaaattcggagccatgatgaaatctggaatgttcctcacactgttt |
| | | gtgaacacagtcattaacattgtaatcgcaagcagagtgttgagagaacgg |
| | | ctaaccggatcaccatgtgcagcattcattggagatgacaatatcgtgaaa |
| | | ggagtcaaatcggacaaattaatggcagacaggtgcgccacctggttgaat |
| | | atggaagtcaagattatagatgctgtggtgggcgagaaagcgccttatttc |
| | | tgtggagggtttattttgtgtgactccgtgaccggcacagcgtgccgtgtg |
| | | gcagaccccctaaaaaggctgtttaagcttggcaaacctctggcagcagac |
| | | gatgaacatgatgatgacaggagaagggcattgcatgaagagtcaacacgc |
| | | tggaaccgagtgggtattctttcagagctgtgcaaggcagtagaatcaagg |
| | | tatgaaaccgtaggaacttccatcatagttatggccatgactactctagct |
| | | agcagtgttaaatcattcagctacctgagaggggcccctataactctctac |
| | | ggctaacctgaatggactacgacatagtctagtccgccaagttcgaaggcg |
| | | cgcctctagagccaccatggaggacgctaaaaatatcaagaagggtcccgc |
| | | tcctttctatccactcgaggacggtaccgctggtgagcaactgcacaaggc |
| | | tatgaaacggtatgctcttgtgccaggcaccatagctttcaccgacgccca |
| | | catagaggtggacattacttacgcagagtactttgagatgtctgtccggct |
| | | ggcagaggcaatgaaaagatatggcctcaacacaaaccataggatagtggt |
| | | ttgcagcgagaattctcttcagttttttatgcccgtcctcggggctctttt |
| | | catcggcgtggctgtcgcccccgccaacgacatatacaacgagcgagagct |
| | | gctgaacagcatgggaatcagtcagcctacagtggtttttgtatcaaagaa |
| | | gggcttgcaaaagatccttaacgtacagaaaaagctccctattattcagaa |
| | | gatcattataatggattcaaagaccgactatcagggattccagtcaatgta |
| | | tactttcgtcacttcccaccttcctcctgggtttaatgaatacgacttcgt |
| | | tcccgaaagtttcgatcgggacaaaactattgcactcatcatgaactcctc |
| | | tgggtccactgggctgcctaaaggtgtcgcacttccccacaggactgcatg |
| | | tgtaaggttctctcacgcacgagatcccatattcggtaaccaaatcattcc |
| | | tgataccgctatcctctctgtagtgcctttccaccatggattcggcatgtt |
| | | caccactctgggttacttgatatgtgggttccgcgtggtccttatgtacag |
| | | atttgaagaggagctctttttgaggagcttgcaggactacaaaatccaaag |
| | | tgctctcctcgtccccacattgttctcattcttcgccaaatctactctcat |
| | | cgacaagtacgatttgagcaacttgcacgaaattgcttctgggggggctcc |
| | | actctccaaagaagtcggcgaagccgttgctaaaaggttccacttgccagg |
| | | cattcgccaggggtatggccttactgagacaacctcagctattctcattac |
| | | ccctgagggtgacgataagcctggtgccgtcggtaaggtagtaccattttt |
| | | cgaggcaaaggtggtcgacttggataccggaaaaaccctcggtgtgaatca |
| | | gagaggcgaattgtgtgtcaggggtccaatgatcatgagcggttacgtcaa |
| | | taatcccgaggccactaacgccttgattgataaagatggttggcttcattc |
| | | aggtgatatagcatactgggatgaggatgaacatttcttcattgtcgatag |
| | | gctcaaaagcctgatcaagtataagggttaccaagtggcccctgccgaact |
| | | cgaatctatactgctgcaacatccaaacatttttgacgctggcgtggccgg |
| | | actgcccgatgatgatgctggtgaactccctgccgccgttgtcgtcctgga |
| | | acatggtaagaccatgacagaaaaggagattgtagattacgtggcttcaca |
| | | agtgactacagctaagaaattgcgcgggggtgtcgttttcgtggacgaggt |
| | | acccaagggcctcactgggaagctcgacgccagaaaaatccgagaaatcct |
| | | gattaaagcaaagaagggcggaaagattgcagtgtagcatcgatgatatcg |
| | | cggccgcatacagcagcaattggcaagctgcttacatagaactcgcggcga |
| | | ttggcatgccgccttaaaatttttattttatttttcttttcttttccgaat |
| | | cggattttgtttttaatatttcaaaaaaaaaaaaaaaa |
| Nucleic Acid | 99 | gctcgctttcttgctgtccaatttctattaaaggttcctttgttccctaag |
| Template, | | tccaactactaaactgggggatattatgaagggccttgagcatctggattc |
| modified | | tgcctaataaaaaacatttattttcattgcaaagccacaccctggagctag |
| mRNA-eGFP- | | caaaaaaaaaaaaaaaaaaaaaaaaaaaaaagcatatgactaaaaaaaaaa |
| Amp resistance | | aaaaaaaaaaaaaaaaaaaaaaaaaaaaaaaaaaaaaaaaaaaaaaaaaaa |
| (SVP-355)- | | aaaaaaaaacgcgtatcgatgatatcgcggccgcatacagcagcaattggc |
| the GOI | | aagctgcttacatagaactcgcggcgattggcatgccgccttaaaattttt |
| (Luciferase, | | attttatttttcttttcttttccgaatcggattttgtttttaatatttcaa |
| SEQ ID NO: | | aaaaaaaaaaaaaaaaaaaaaaaacgcgtgataacgcaggaaagaacatgt |
| 25) is bolded. | | gagcaaaaggccagcaaaaggccaggaaccgtaaaaaggccgcgttgctgg |
| The GOI can | | cgtttttccataggctccgcccccctgacgagcatcacaaaaatcgacgct |
| be substituted | | caagtcagaggtggcgaaacccgacaggactataaagataccaggcgtttc |
| with one or | | cccctggaagctccctcgtgcgctctcctgttccgaccctgccgcttaccg |
| more different | | gatacctgtccgcctttctcccttcgggaagcgtggcgctttctcatagct |
| GOIs. | | cacgctgtaggtatctcagttcggtgtaggtcgttcgctccaagctgggct |
| | | gtgtgcacgaaccccccgttcagcccgaccgctgcgccttatccggtaact |
| | | atcgtcttgagtccaacccggtaagacacgacttatcgccactggcagcag |
| | | ccactggtaacaggattagcagagcgaggtatgtaggcggtgctacagagt |
| | | tcttgaagtggtggcctaactacggctacactagaagaacagtatttggta |
| | | tctgcgctctgctgaagccagttaccttcggaaaaagagttggtagctctt |
| | | gatccggcaaacaaaccaccgctggtagcggtttttttgtttgcaagcagc |
| | | agattacgcgcagaaaaaaaggatctcaagaagatcctttgatcttttcta |
| | | cggggtctgacgctcagtggaacgaaaactcacgttaagggattttggtca |
| | | tgagattatcaaaaaggatcttcacctagatccttttaaattaaaaatgaa |
| | | gttttaaatcaatctaaagtatatatgagtaaacttggtctgacagttacc |
| | | aatgcttaatcagtgaggcacctatctcagcgatctgtctatttcgttcat |
| | | ccatagttgcctgactccccgtcgtgtagataactacgatacgggagggct |
| | | taccatctggccccagtgctgcaatgataccgcgagacccacgctcaccgg |
| | | ctccagatttatcagcaataaaccagccagccggaagggccgagcgcagaa |
| | | gtggtcctgcaactttatccgcctccatccagtctattaattgttgccggg |
| | | aagctagagtaagtagttcgccagttaatagtttgcgcaacgttgttgcca |
| | | ttgctacaggcatcgtggtgtcacgctcgtcgtttggtatggcttcattca |
| | | gctccggttcccaacgatcaaggcgagttacatgatcccccatgttgtgca |
| | | aaaaagcggttagctccttcggtcctccgatcgttgtcagaagtaagttgg |
| | | ccgcagtgttatcactcatggttatggcagcactgcataattctcttactg |
| | | tcatgccatccgtaagatgcttttctgtgactggtgagtactcaaccaagt |
| | | cattctgagaatagtgtatgcggcgaccgagttgctcttgcccggcgtcaa |
| | | tacgggataataccgcgccacatagcagaactttaaaagtgctcatcattg |
| | | gaaaacgttcttcggggcgaaaactctcaaggatcttaccgctgttgagat |
| | | ccagttcgatgtaacccactcgtgcacccaactgatcttcagcatctttta |
| | | ctttcaccagcgtttctgggtgagcaaaaacaggaaggcaaaatgccgcaa |
| | | aaaagggaataagggcgacacggaaatgttgaatactcatactcttccttt |
| | | ttcaatattattgaagcatttatcagggttattgtctcatgagcggataca |
| | | tatttgaatgtatttagaaaaataaacaaataggggttccgcgcacatttc |
| | | cccgaaaagtgccacctgagctctaatacgactcactataggacatttgct |
| | | tctgacacaactgtgttcactagcaacctcaaacagacaccatggtgagca |
| | | agggcgaggagctgttcaccggggtggtgcccatcctggtcgagctggacg |
| | | gcgacgtaaacggccacaagttcagcgtgtccggcgagggcgagggcgatg |
| | | ccacctacggcaagctgaccctgaagttcatctgcaccaccggcaagctgc |
| | | ccgtgccctggcccaccctcgtgaccaccctgacctacggcgtgcagtgct |
| | | tcagccgctaccccgaccacatgaagcagcacgacttcttcaagtccgcca |
| | | tgcccgaaggctacgtccaggagcgcaccatcttcttcaaggacgacggca |
| | | actacaagacccgcgccgaggtgaagttcgagggcgacaccctggtgaacc |
| | | gcatcgagctgaagggcatcgacttcaaggaggacggcaacatcctggggc |
| | | acaagctggagtacaactacaacagccacaacgtctatatcatggccgaca |
| | | agcagaagaacggcatcaaggtgaacttcaagatccgccacaacatcgagg |
| | | acggcagcgtgcagctcgccgaccactaccagcagaacacccccatcggcg |
| | | acggccccgtgctgctgcccgacaaccactacctgagcacccagtccgccc |
| | | tgagcaaagaccccaacgagaagcgcgatcacatggtcctgctggagttcg |
| | | tgaccgccgccgggatcactctcggcatggacgagctgtacaagtag |
| Nucleic Acid | 100 | gctcgctttcttgctgtccaatttctattaaaggttcctttgttccctaag |
| Template, | | tccaactactaaactgggggatattatgaagggccttgagcatctggattc |
| modified | | tgcctaataaaaaacatttattttcattgcaaagccacaccctggagctag |
| mRNA- | | caaaaaaaaaaaaaaaaaaaaaaaaaaaaaagcatatgactaaaaaaaaaa |
| Luciferase- | | aaaaaaaaaaaaaaaaaaaaaaaaaaaaaaaaaaaaaaaaaaaaaaaaaaa |
| Amp resistance | | aaaaaaaaacgcgtatcgatgatatcgcggccgcatacagcagcaattggc |
| (SVP-062)- | | aagctgcttacatagaactcgcggcgattggcatgccgccttaaaattttt |
| the GOI | | attttatttttcttttcttttccgaatcggattttgtttttaatatttcaa |
| (Luciferase, | | aaaaaaaaaaaaaaaaaaaaaaaacgcgtgataacgcaggaaagaacatgt |
| SEQ ID NO: | | gagcaaaaggccagcaaaaggccaggaaccgtaaaaaggccgcgttgctgg |
| 25) is bolded. | | cgtttttccataggctccgcccccctgacgagcatcacaaaaatcgacgct |
| The GOI can | | caagtcagaggtggcgaaacccgacaggactataaagataccaggcgtttc |
| be substituted | | cccctggaagctccctcgtgcgctctcctgttccgaccctgccgcttaccg |
| with one or | | gatacctgtccgcctttctcccttcgggaagcgtggcgctttctcatagct |
| more different | | cacgctgtaggtatctcagttcggtgtaggtcgttcgctccaagctgggct |
| GOIs. | | gtgtgcacgaaccccccgttcagcccgaccgctgcgccttatccggtaact |
| | | atcgtcttgagtccaacccggtaagacacgacttatcgccactggcagcag |
| | | ccactggtaacaggattagcagagcgaggtatgtaggcggtgctacagagt |
| | | tcttgaagtggtggcctaactacggctacactagaagaacagtatttggta |
| | | tctgcgctctgctgaagccagttaccttcggaaaaagagttggtagctctt |
| | | gatccggcaaacaaaccaccgctggtagcggtttttttgtttgcaagcagc |
| | | agattacgcgcagaaaaaaaggatctcaagaagatcctttgatcttttcta |
| | | cggggtctgacgctcagtggaacgaaaactcacgttaagggattttggtca |
| | | tgagattatcaaaaaggatcttcacctagatccttttaaattaaaaatgaa |
| | | gttttaaatcaatctaaagtatatatgagtaaacttggtctgacagttacc |
| | | aatgcttaatcagtgaggcacctatctcagcgatctgtctatttcgttcat |
| | | ccatagttgcctgactccccgtcgtgtagataactacgatacgggagggct |
| | | taccatctggccccagtgctgcaatgataccgcgagacccacgctcaccgg |
| | | ctccagatttatcagcaataaaccagccagccggaagggccgagcgcagaa |
| | | gtggtcctgcaactttatccgcctccatccagtctattaattgttgccggg |
| | | aagctagagtaagtagttcgccagttaatagtttgcgcaacgttgttgcca |
| | | ttgctacaggcatcgtggtgtcacgctcgtcgtttggtatggcttcattca |
| | | gctccggttcccaacgatcaaggcgagttacatgatcccccatgttgtgca |
| | | aaaaagcggttagctccttcggtcctccgatcgttgtcagaagtaagttgg |
| | | ccgcagtgttatcactcatggttatggcagcactgcataattctcttactg |
| | | tcatgccatccgtaagatgcttttctgtgactggtgagtactcaaccaagt |
| | | cattctgagaatagtgtatgcggcgaccgagttgctcttgcccggcgtcaa |
| | | tacgggataataccgcgccacatagcagaactttaaaagtgctcatcattg |
| | | gaaaacgttcttcggggcgaaaactctcaaggatcttaccgctgttgagat |
| | | ccagttcgatgtaacccactcgtgcacccaactgatcttcagcatctttta |
| | | ctttcaccagcgtttctgggtgagcaaaaacaggaaggcaaaatgccgcaa |
| | | aaaagggaataagggcgacacggaaatgttgaatactcatactcttccttt |
| | | ttcaatattattgaagcatttatcagggttattgtctcatgagcggataca |
| | | tatttgaatgtatttagaaaaataaacaaataggggttccgcgcacatttc |
| | | cccgaaaagtgccacctgagctctaatacgactcactataggacatttgct |
| | | tctgacacaactgtgttcactagcaacctcaaacagacaccatggaggacg |
| | | ctaaaaatatcaagaagggtcccgctcctttctatccactcgaggacggta |
| | | ccgctggtgagcaactgcacaaggctatgaaacggtatgctcttgtgccag |
| | | gcaccatagctttcaccgacgcccacatagaggtggacattacttacgcag |
| | | agtactttgagatgtctgtccggctggcagaggcaatgaaaagatatggcc |
| | | tcaacacaaaccataggatagtggtttgcagcgagaattctcttcagtttt |
| | | ttatgcccgtcctcggggctcttttcatcggcgtggctgtcgcccccgcca |
| | | acgacatatacaacgagcgagagctgctgaacagcatgggaatcagtcagc |
| | | ctacagtggtttttgtatcaaagaagggcttgcaaaagatccttaacgtac |
| | | agaaaaagctccctattattcagaagatcattataatggattcaaagaccg |
| | | actatcagggattccagtcaatgtatactttcgtcacttcccaccttcctc |
| | | ctgggtttaatgaatacgacttcgttcccgaaagtttcgatcgggacaaaa |
| | | ctattgcactcatcatgaactcctctgggtccactgggctgcctaaaggtg |
| | | tcgcacttccccacaggactgcatgtgtaaggttctctcacgcacgagatc |
| | | ccatattcggtaaccaaatcattcctgataccgctatcctctctgtagtgc |
| | | ctttccaccatggattcggcatgttcaccactctgggttacttgatatgtg |
| | | ggttccgcgtggtccttatgtacagatttgaagaggagctctttttgagga |
| | | gcttgcaggactacaaaatccaaagtgctctcctcgtccccacattgttct |
| | | cattcttcgccaaatctactctcatcgacaagtacgatttgagcaacttgc |
| | | acgaaattgcttctgggggggctccactctccaaagaagtcggcgaagccg |
| | | ttgctaaaaggttccacttgccaggcattcgccaggggtatggccttactg |
| | | agacaacctcagctattctcattacccctgagggtgacgataagcctggtg |
| | | ccgtcggtaaggtagtaccatttttcgaggcaaaggtggtcgacttggata |
| | | ccggaaaaaccctcggtgtgaatcagagaggcgaattgtgtgtcaggggtc |
| | | caatgatcatgagcggttacgtcaataatcccgaggccactaacgccttga |
| | | ttgataaagatggttggcttcattcaggtgatatagcatactgggatgagg |
| | | atgaacatttcttcattgtcgataggctcaaaagcctgatcaagtataagg |
| | | gttaccaagtggcccctgccgaactcgaatctatactgctgcaacatccaa |
| | | acatttttgacgctggcgtggccggactgcccgatgatgatgctggtgaac |
| | | tccctgccgccgttgtcgtcctggaacatggtaagaccatgacagaaaagg |
| | | agattgtagattacgtggcttcacaagtgactacagctaagaaattgcgcg |
| | | ggggtgtcgttttcgtggacgaggtacccaagggcctcactgggaagctcg |
| | | acgccagaaaaatccgagaaatcctgattaaagcaaagaagggcggaaaga |
| | | ttgcagtgtag |
| GOI, human | 101 | atggacatgagagtcccagctcaactgcttggtcttcttctgctgtggttg |
| IL18-mutant- | | cgcggtgccagatgtcacttcgggaagttggagtccaagttgagtgttatt |
| the leading | | cgcaacctcaatgatcaggtgttgttcatagaccagggtaaccgcccactt |
| peptide is | | tttgaagatatgaccgatagcgattgccgcgacaacgcccccaggaccatc |
| bolded. | | ttcataataagtgcgtacggagactcacgggcccgggggaaggctgttaca |
| | | atatccgtaaagtgcgagaagatctccacgctttcttgcgaaaataagatt |
| | | atcagttttaaagaaatgaaccctcctgacaacataaaagatacgaaatca |
| | | gacatcatcttcttccagcgctcagttcctggacatgataataagatgcag |
| | | ttcgagtcatctagctatgaagggtattttctggcttgtgagaaagaacgg |
| | | gatttgtttaaacttatactgaagaaggaggatgaacttggtgatcggtct |
| | | attatgtttaccgtacaaaatgaggac |
| GOI, human | 102 | atggacatgagagtcccagctcaactgcttggtcttcttctgctgtggttg |
| IL18-the | | cgcggtgccagatgtcacttcgggaagttggagtccaagttgagtgttatt |
| leading peptide | | cgcaacctcaatgatcaggtgttgttcatagaccagggtaaccgcccactt |
| is bolded. | | tttgaagatatgaccgatagcgattgccgcgacaacgcccccaggaccatc |
| | | ttcataataagtgcgtacggagactcacgggcccgggggaaggctgttaca |
| | | atatccgtaaagtgcgagaagatctccacgctttcttgcgaaaataagatt |
| | | atcagttttaaagaaatgaaccctcctgacaacataaaagatacgaaatca |
| | | gacatcatcttcttccagcgctcagttcctggacatgataataagatgcag |
| | | ttcgagtcatctagctatgaagggtattttctggcttgtgagaaagaacgg |
| | | gatttgtttaaacttatactgaagaaggaggatgaacttggtgatcggtct |
| | | attatgtttaccgtacaaaatgaggac |
| IRES | 103 | acgttactggccgaagccgcttggaataaggccggtgtgcgtttgtctata |
| | | tgttattttccaccatattgccgtcttttggcaatgtgagggcccggaaac |
| | | ctggccctgtcttcttgacgagcattcctaggggtctttcccctctcgcca |
| | | aaggaatgcaaggtctgttgaatgtcgtgaaggaagcagttcctctggaag |
| | | cttcttgaagacaaacaacgtctgtagcgaccctttgcaggcagcggaacc |
| | | ccccacctggcgacaggtgcctctgcggccaaaagccacgtgtataagata |
| | | cacctgcaaaggcggcacaaccccagtgccacgttgtgagttggatagttg |
| | | tggaaagagtcaaatggctctcctcaagcgtattcaacaaggggctgaagg |
| | | atgcccagaaggtaccccattgtatgggatctgatctggggcctcggtgca |
| | | catgctttacatgtgtttagtcgaggttaaaaaaacgtctaggccccccga |
| | | accacggggacgtggttttcctttgaaaaacacgataata |
| Upper linker of | 104 | gcgatcgcgctagcaacggtttccctctagcgggatcaattccgccccccc |
| IRES | | ccccta |
| Down linker of | 105 | acgcgttgccacc |
| IRES | | |
| mouse mutant | 106 | atgacctcccggcttgtgagggtactggctgctgctatgctggtggctgct |
| IL-18 | | gctgtgagtgtggcacacttcggacgactccattgtactacagccgttata |
| | | cgaaatatcaatgatcaggtgttgtttgtagacaaaaggcagcccgtgttt |
| | | gaagacatgactgatattgatcagagtgcctctgagccccagactagactg |
| | | attatatatgcttatggcgactctagagctagagggaaagcagtaactctg |
| | | tcagtgaaagacagcaagatgtctaccctgtcttgcaagaataaaataatc |
| | | tcatttgaggagatggacccccctgaaaacatagatgacatccaaagtgac |
| | | ctgatattctttcagaagcgggtacccggtcataacaaaatggagttcgag |
| | | tcaagcctctatgagggtcactttctcgcctgccaaaaggaagatgacgcc |
| | | ttcaaattgatcttgaaaaaaaaagacgagaatggtgataagtctgtcatg |
| | | ttcacactcactaatttgcatcagtca |
| mouse wild | 107 | atgacctcccggcttgtgagggtactggctgctgctatgctggtggctgct |
| type IL-18 | | gctgtgagtgtggcaaactttgggagactccactgtaccactgccgttata |
| | | cggaatataaacgatcaggtcctctttgtagacaagagacagccagttttc |
| | | gaggacatgactgatatagatcagagtgcctctgagcctcaaactcggctc |
| | | ataatatacatgtataaggatagcgaggttcggggacttgccgtgactttg |
| | | tccgtgaaagactcaaaaatgagcaccctgtcatgcaaaaataagataatt |
| | | tctttcgaggaaatggaccctcccgagaacattgatgatatccagtccgac |
| | | ttgatatttttccagaaacgggttcccggtcataacaaaatggagtttgag |
| | | tcttcattgtacgagggtcattttttggcatgccagaaggaggatgatgcc |
| | | ttcaaactgattttgaagaaaaaagacgagaacggagacaagtctgtcatg |
| | | tttacacttaccaaccttcatcaatcc |
| Capsids/envelop | 108 | atgaatagaggaatatttaacatgctgagccgccgccccttccccgccccc |
| from SIN | | acagctatgtggaggcctaggcgaaggaggcaggcggcaccactgcccgcc |
| virus | | cgtaatgggttggcatcccaaattcaacagctaaccacagctgtcagtgcc |
| | | ctagtcattggtcaagccacccgacctcaacaaccaaggccacgcccgcta |
| | | ccacgccctaagaagcagtccccgaagcaaccaccgaatgctaaaaagaca |
| | | aaaccgcaggagaacaaggagaaacaacccgcaaaaccgaagccgggtaag |
| | | agacagcgtatggcactaaaattgatgctgacaggactctttgacgtgaac |
| | | aacgaagccggtgacgtaataggacacgccctcgctatggaaggcaaagtg |
| | | atgaagccacttcacgtgaaaggggtcatagaccacccggttttggcaaag |
| | | ctgaaatttaccaagtcatcagcgtacgacatggagttcgcccagcttcca |
| | | gccagtatgaaaagcgaagcatttgcatacaccagcgagcaccctgaaggc |
| | | ttctacaactggcatcatggtgccgtgcagtacagcggcggtcgctttaca |
| | | gtcccgcgcggagttgggggaaaaggcgacagtggaagaccgatcatggat |
| | | aatacaggtaaggtggtcgcgatagtgctgggcggcgctgacgaaggcgcc |
| | | cgcactgctctctccgtcgtcacttggaacagcaaaggaaagaccatcaaa |
| | | acgactccggaagggactgaggaatggtctgcagcaccattagtcacggct |
| | | atgtgcttctttgagaatgtatcgttcccgtgcagtcgtccgcctacgtgc |
| | | tattcccgaaaaccatctcgtgcccttgacatcctggaggagaacgtgaac |
| | | aacgacgcctacgataccttattagcggcagtgttgaagtgcaggacgtcc |
| | | ggtcggaacaaacgcagcatcaccgacgatttcaccctgaccagcccgtac |
| | | ctgggcacttgctcgtattgccatcatactgaaccctgcttcagcccaatt |
| | | aagatcgagcaagtttgggacgaggcggacgacggatcaatacgaatacag |
| | | acatccgcccagttcgggtacgaccaaagtggggcagcagacgtcaacaag |
| | | tacaggtacatgtcaattgaccaggaccatgccgtgaaagaaggctctatg |
| | | gatcatatcaagatcagcacatccgggccctgtaggcggcttcaatcacaa |
| | | aggtacttcttgcttgcgaaatgcccaccgggagatagcgttacggttagc |
| | | ataaccggcggcactatggccacctcctgtacattagcacgtaaaataagg |
| | | caaaaattcgtaggacgtgagaagtatgaccttcccccagcacacggaaag |
| | | aacatcccctgcaccgtgtatgatcgtcttaaggagacgtcagccggatac |
| | | atcaccatgcacaggcctggacctcatgcctatacctcatacctagaggct |
| | | acctcaggaaaaatttacgccaaaccaccatcaggaaagaacatcacgtac |
| | | gagtgtaagtgtggcgattataaaactgcgaccgtttccgtcagaacagag |
| | | atcgcaggatgtactgccataaaacagtgcgtggcgtacaagagcgaccag |
| | | accaagtgggtgttcaactcgcccgatctaatcaggcacgctgaccacaca |
| | | gcccaaggtaaattccactttcctttcaagcctaccttcagtacatgcctg |
| | | gtaccactggcacacgagccgactgtaattcatgggttcaaacacataagc |
| | | ctccatttagacacagaccacccaaccttgctcaccattagaagactgggc |
| | | gagaaacccgaaccgacgtcggagtggatctcaggaaaaacagttaggaat |
| | | tttactgtcgacagagacgggctagagtatatatggggcaatcacgaaccg |
| | | gtgagagtatatgcacaagagtccgcccccggtgaccctcacggatggcca |
| | | cacaaaataattcaacactactatcaacgccacccagtatacactagcttg |
| | | gctgtagcagcttccgctgtggcggtgatagtcggcctggccaccgccgtg |
| | | atgtgtatctgcagagcgcgacgggagtgcctaaccccgtacgcgttagcc |
| | | ccgaatgcagtcatcccgacgtccttggccttattgtgctgcgttcggtcc |
| | | gccaacgccgaatctttcaccgacacgatgggatacctatggtcgaacagt |
| | | caaacatttttctgggtgcagctgtgcatccccttggcagtagtaatagta |
| | | ctcgtacgctgctgttcgtgctgcctcccttttttagtggttgcaggcgcc |
| | | tacctggcgaaggtagacgccttcgaacatgcgaccactgtcccaaatgtg |
| | | ccgaagattccgtataaagctttggtcgagagagccggatacgccccgctt |
| | | aacttggaaattactgttatgtcttcagaggtcctcccttcgattcaccaa |
| | | gaatacatcacctgcaaatactccactatcgtgccttccccgaaagtgaaa |
| | | tgctgcggttcccttgagtgccaacccgcttcgcatgccgactacaactgc |
| | | aaagtatttggcggcgtttaccctttcatgtggggtggagcgcaatgtttt |
| | | tgcgacagcgagaacagccagatgagtgaggcgtatgtcgagctatcggcc |
| | | gattgtgcccttgatcacgcacaggcggtgaaagtgcacacagccgcaatg |
| | | aaggtcggactacgcattgtgtatgggaacaccactagcctcctggatgcg |
| | | tacgtaaacggagtcacgccaggcacatctaaagacctgaaagtcattgcc |
| | | ggacccatctcgtccgcgttttccccttttgactctaaagtcgtgatacac |
| | | cgagggctcgtgtacaattacgattttccagagtacggggcaatgaaaccg |
| | | ggtgcctttggggacattcaagctagctctttgacgggcaacgacctgatt |
| | | gccagcaccgacatacgtttgcttaaacccgcagcaaagaatgtgcacgtt |
| | | ccctatacccaagcagcatcaggatttgaaatttggaagaacaactccgga |
| | | agaccactgcaagagacggccccatttggctgtaagattgccgtcaaccct |
| | | ctgaaggccgtggattgttcctatgggaacatcccgatatcgattgatatc |
| | | ccccacgccgcctttattagaccatccgatgcgccatttatctccgaagtg |
| | | aagtgcgaagtcagcgactgcacatactctgctgacttcggaggcatggcc |
| | | accctacagtatgtgtctgaccgagagggccagtgcccagtgcattcccat |
| | | tcaagtaccgccacactccaggaatcaactgttcacgtcttggaaaaaggg |
| | | gcgacgacagtgcacttcagcacagctagccctcacgccagctttgtggtc |
| | | tcattatgcggaaagaagacgacctgcaccgcaggatgcaaaccacctgtc |
| | | ggccacatagtcagcacaccccacaagaaggaccaagagttccagaccgcc |
| | | gtctccaggacatcatggagctggctcttcgcgctgttcggaggagcttcg |
| | | tccctagtaatcatgggactgttaatcttctcctgcagcatgatgctcact |
| | | aacaccagaaga |

All of the foregoing sequences, components, molecules and methods, and any and all combinations thereof are non-limiting examples and aspects of the present disclosure.

In addition, it is to be understood that any particular aspect of the present disclosure that falls within the prior art may be explicitly excluded from any one or more of the claims. Since such aspects are deemed to be part of the whole of the present disclosure, any part of the whole disclosure may be excluded even if the exclusion is not set forth explicitly herein.

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A composition for cascade amplifications of therapeutic payloads (CATP) comprising:
a) a first type nucleic acid construct encoding a self-amplifying mRNA (sa-mRNA) or multiple self-amplifying mRNA (sa-mRNA) encoding at least one gene of interest (GOI) or a plurality of GOIs;
b) a second type nucleic acid construct encoding an mRNA encoding at least one virus structural protein; and
c) at least one payload delivery system,
wherein the at least one payload delivery system is a non-viral payload delivery system and
wherein the payload is at least one nucleic acid construct.

2. The composition of claim 1, wherein the at least one virus structural protein encodes a viral capsid protein, a viral envelope protein, or a combination thereof.

3. The composition of claim 1, wherein the first nucleic acid construct or the second nucleic acid construct is a mRNA molecule.

4. The composition of claim 1, wherein the first nucleic acid construct or the second nucleic acid construct is a DNA construct.

5. The composition of claim 1, wherein the first type nucleic acid construct is a sa-mRNA molecule or multiple sa-mRNA molecules.

6. The composition of claim 4, wherein the second nucleic acid construct is a mRNA molecule.

7. The composition of claim 1, wherein the non-viral payload delivery system is a lipidoid, a polymer, a core-shell nanoparticle, a nanoparticle mimic, a lipid nanoparticle (LNP), a polymeric nanoparticle, a micelle, liposome, an exosome, a virus-like particle (VLP), a lipoplex, a polyplex, a lipopolyplex, or a microbe.

8. The composition of claim 7, wherein the LNP comprises an ionizable lipid compound selected from Formulae I, II, IV, V, IV, and VII, wherein Formula I is 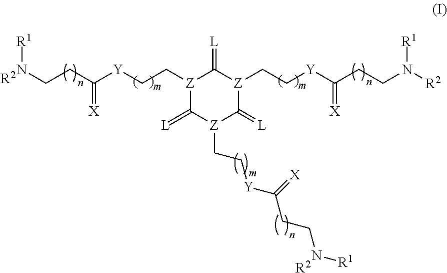
or a salt or isomer thereof;
wherein Formula II is 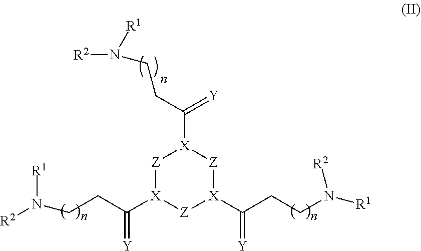
or a salt or isomer thereof;
wherein Formula IV is 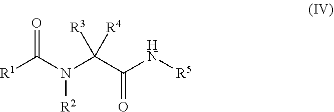
or a salt or isomer thereof;
wherein Formula V is 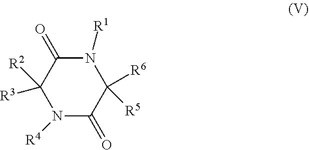
or a salt or isomer thereof;
wherein Formula VI is 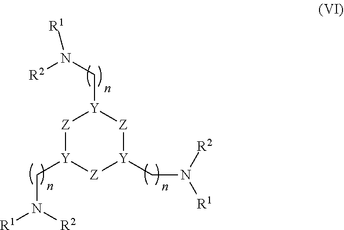
or a salt or isomer thereof;
wherein Formula VII is 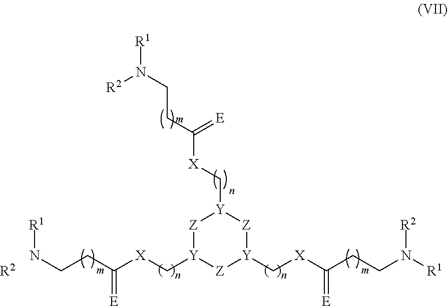
or a salt or isomer thereof;
wherein, each m and n are independently an integer from 0-10;
each R¹, R², R³, R⁴, R⁵ and R⁶ are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, 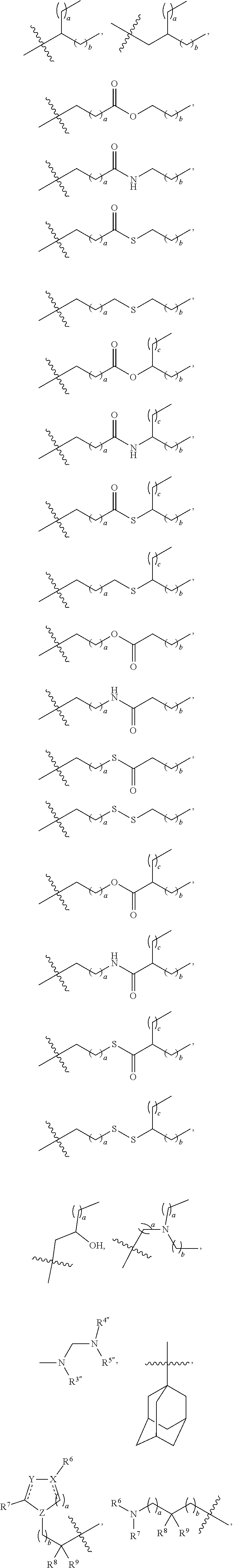
a, b and c are each independently an integer from 0-24;
each R^{6′}, R⁷, R⁸ and R⁹ are independently selected from H, C1-C24 alkyl, C1-C24 alkenyl, C1-C24 alkynyl, substituted alkyl, substituted alkenyl, substituted alkynyl, substituted acyl, substituted carbocyclyl, substituted heterocyclyl, substituted aryl, substituted heteroaryl, 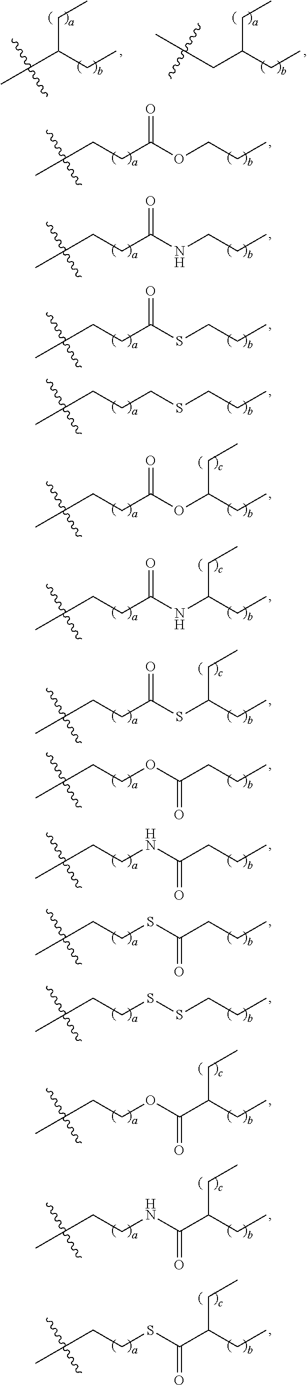 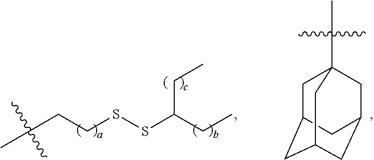
each R^{3′}, R^{4′} and R^{5′} are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, 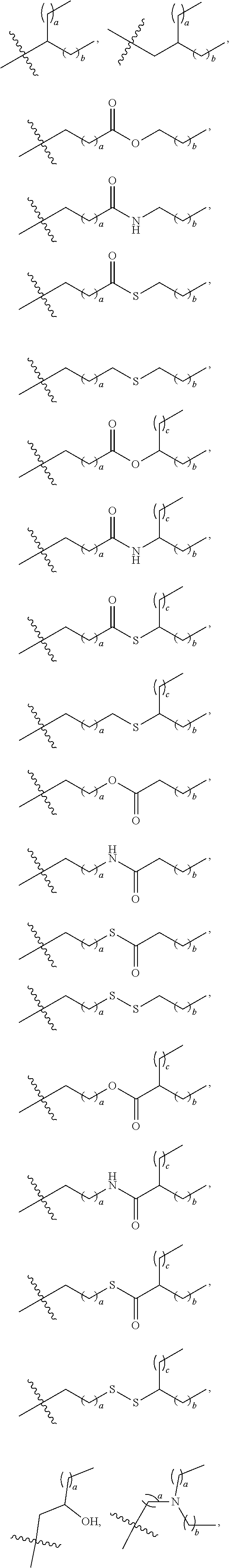
a, b and c are each independently an integer from 0-24;
each X is independently selected from CH₂, NH, O, or S;
each Y is independently selected from CH₂, NH, O, or S;
each Z is independently selected from CH, N, CH₂, NH, O, or S; and
each E is independently selected from CH₂, NH, O, or S.

9. The composition of claim 7, wherein the LNP comprises an ionizable lipid compound selected from Formula III, wherein Formula III is 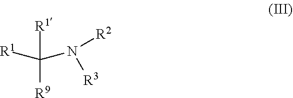
or a salt or isomer thereof, wherein
each n is independently an integer from 0-10;
each R¹, R^{1′}, R² and R³ are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, 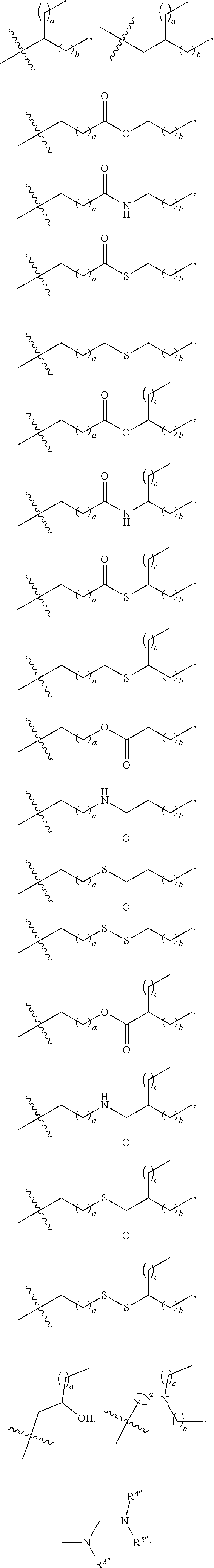
a, b and c are each independently an integer from 0-24,
each R⁹ is independently selected from 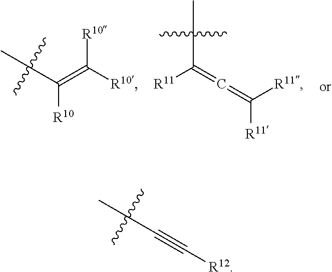
wherein each R¹⁰, R^{10′}, R^{10″}, R¹¹, R^{11′}, R^{11″}, and R¹² are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, 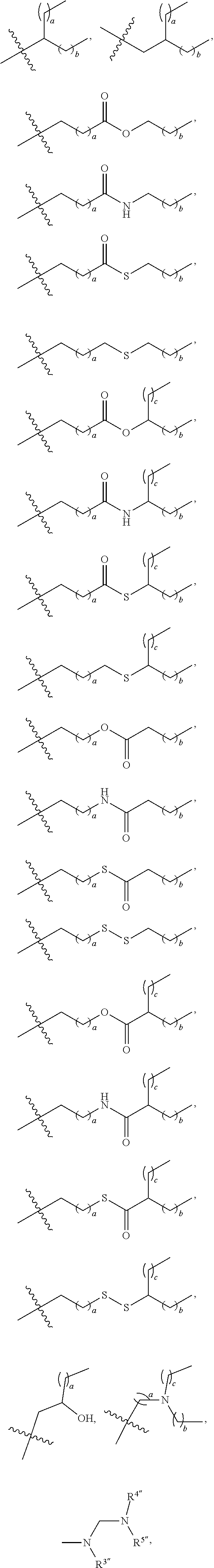
a, b and c are each independently an integer from 0-24;
R^{3″}, R^{4″}, and R^{5″} are independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, 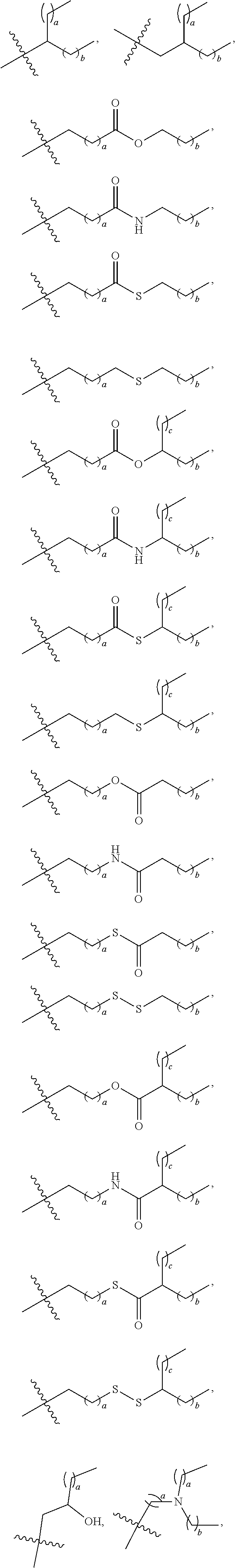
a, b and c are each independently an integer from 0-24.

10. The composition of claim 7, wherein the LNP comprises an ionizable lipid compound, wherein the ionizable lipid is (Ia), (Ib), (Iia), (Iib), (IIIa), (IIIb), (IIIc), (IIId), (IIIe), (IIIf), (IIIg), (IIIh), (IIIi), (IIIk), (IIIl), (IIIm), (IIIn), (IIIo), (IIIp), (IIIq), (IIIr), (IIIs), (IIIt), (IIIu), (IIIv), (IIIw), (IIIx), (IIIy), (IIIz), (III2), (III3), (III4), (IVa), (IVb), (IVc), (IVd), (IVe), (IV), (IVg), (IVh), (IVi), (IVk), (IVl), (IVm), (IVn), (IVo), (IVp), (IVq), (IVr), (IVs), (IVt), (IVu), (IVv), (IVw), (IVx), (IVy), (IVz), (IVaa), (IVab), (IVac), (IVad), (IVae), (Va), (Vb), (VIa), (VIb), (VIc), (VIIa), (VIIb), (P501C1), (P501G3), (P501G6), (P501C1), (P501G3), (P501G6), (HP9E9), (HP9G3), (HP9G4), (HP9G6), (HP9G7), (HP9G8), (HP9G9), (HP9G10), (HP10G6), (HP11E7), (HP11E8), (HP11G4), (HP12F4), (HP12F6), (HP12G7), (HP13B9), (HP13C6), (HP13F10), (HP13G8), (HP15A4), (HP15C2), (HP15C4), (HP15E3), (HP15E4), (HP15E6), (HP15E7), (HP15E8), (HP15G5), (HP16B6), (HP16C4), (HP16C6), (HP16G6), (HP17E7), (HP17G6), (HP18E7), (HP19E2), (HP19E6), (HP19E8), (HP19G6), (HP19G7), (HP19G8), (HP19G9), (HP19G10), (HP20A10), (HP20F7), (HP20F8), (HP20F10), (HP20G3), (HP20G10), (P502B9), (P502D5), (P502D10), (P502F10), (P502H5), (P502H7), (P502H9), (P502H10), (P502H11), (P503B7), (P503B9), (P503B10), (P503H7), (P503H10), (P504B10), (P504D4), (P504D5), (P504D7), (P504D9), (P504D11), (P504F9), (P504F10), (P504H4), (P504H7), (P504H9), (P505B4), (P505B7), (P505B9), (P505B10), (P505C10), (P505D4), (P505D7), (P505D9), (P505D10), (P505D11), (P505H7), (P505H9), (P506D7), (P506D10), (P509D5), (P509D7), (P509D9), (P509D10), (P510B2), (P510B6), (P510D1), (P510D2), (P510D3), (P510D4), (P510D5), (P510D6), (P510D7), (P510D9), (P510D10), (P510D11), (P510D12), (P510F7), (P510H3), (P510H4), (P510H5), (P510H7), (P510H11), (P510H12), (P511D1), (P511D2), (P511D3), (P511D7), (P511D11), (P511D12), (P511F5), (P511F6), (P511F7), (P511F11), (P511F12), (P511H1), (P511H2), (P511H3), (P511H5), (P511H6), (P511H7), (P511H9), (P511H11), (P511H12), (P512A9), (P512A10), (P512A11), (P512A12), (P513A9), (P513A10), (P514A1), (P514A2), (P514A3), (P514B1), (P514B2), (P514A7), (P514A9), (P514A10), (P514B8), (P514B9), (P515A11), (P515A12), (P515B10), (P515B11), (P515B12), (P516A7), (P516A8), (P516A9), (P516B7), (P516B9), (P517A7), (P518A7), (P518A9), (P518A10), (P518B7), (P518B8), (P518B9), (P518B11), (P519A9), (P519B7), (P519B8), (P519B9), (P520B7), (P520B8), (P520B9), (P520B10), (P521B9), (P521B10), (P521B11), (P521B12), (P522B9), (P522B10), (P522B11), (P522B12), (P536B3), (P536B4), (P537B2), (P537B3), (P537B4), (P538B2), (P538B3), (P538B4), (P538C2), (P540B2), (P540B3), (P540B4), (P541B2), (P541B3), (P541B4), (P541B9), (P542B2), (P542B3), (P542B4), (P542B9), (P543B3), (P543B4), (P544B3), (P544B4), (P551B8), (P551B9), (P554B9), (P554B10), (P556A11), (P556B3), (P556B9), (P558A11), (P558B9), (P559A11), (P559B3), (P559B4), (P559B9), (P559B10), (P560B3), (P560B4), (P560B5), (P560B9), (P560B10), (P560B11), (P561A11), (P561B4), (P561B5), (P561B9), (P562A11), (P562B3), (P562B4), (P562B5), (P562B9), (P563A5), (P563A11), (P563B3), (P563B4), (P563B5), (P563B9), (P563B10), (P563B11), (P564A11), (P564B2), (P564B3), (P564B4), (P564B5), (P564B9), (P564B10), (P565B3), (P565B4), (P565B5), (P565B9), (P565B10), (P565B11), (P566B3), (P566B4), (P566B10), (P569A9), (P569A10), (P569B10), (P570A8), (P570A9), (P570A10), (P571A8), (P571A9), (P571B2), (P572A9), (P572A10), (P572B2), (P573A8), (P573A9), (P573A10), (P573B2), (P573B4), (P573B9), (P573B10), (P574A8), (P574A9), (P574A10), (P574B2), (P574B3), (P574B9), (P574B10), (P575A9), (P575A10), (P575B9), (P575B10), (P576A4), (P576A8), (P576A9), (P576A10), (P576B3), (P576B4), (P576B9), (P576B10), (P577B3), (P577B4), (P584B3), (P584B4), (P585B3), (P586B3), (P586B4), (P589B3), (P589B4), (P590B4), (P591B2), (P591B3), (P591B4), (P591E4), (P592A4), (P592B2), (P592B3), (P592B4), (P592B5), (P592E3), (P593B2), (P593B3), (P593B4), (P593B5), (P593E2), (P593E9), (P593H3), (P593H4), (P594B10), (P594B5), (P595B2), (P595B3), (P595B4), (P595B5), (P595E2), (P595E3), (P595E4), (P595H4), (P596B2), (P596B3), (P596B4), (P596B5), (P596E3), (P596E4), (P596H3), (P597A4), (P597B2), (P597B3), (P597B4), (P597B5), (P597B6), (P597D4), (P597E2), (P597E3), (P597E4), (P597H4), (P598B1), (P598B2), (P598B3), (P598B10), (P598B5), (P598E2), (P598E3), (P598E4), (P598H3), (P598H4), (P599B2), (P599B3), (P599B4), (P617F12), (P618H5), (P623A5), (P623B8), (P625A6), (P625A9), (P625F8), (P625F9), (P625H8), (P627E10), or salt or isomer thereof.

11. The composition of claim 7, wherein the LNP comprises a modular lipid compound selected from Formulae VIII-IX, wherein Formula VIII is 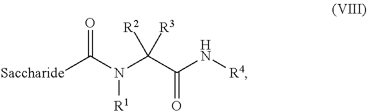
or a salt or isomer thereof;
wherein Formula IX is 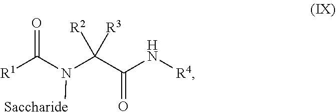
or a salt or isomer thereof;
wherein
each of R¹, R², R³ and R⁴ is independently selected from H, C1-C24 alkyl, C1-C24 alkenyl, C1-C24 alkynyl, substituted alkyl, substituted alkenyl, substituted alkynyl, substituted acyl, substituted carbocyclyl, substituted heterocyclyl, substituted aryl, substituted heteroaryl, poly(ethylene glycol) (PEG), 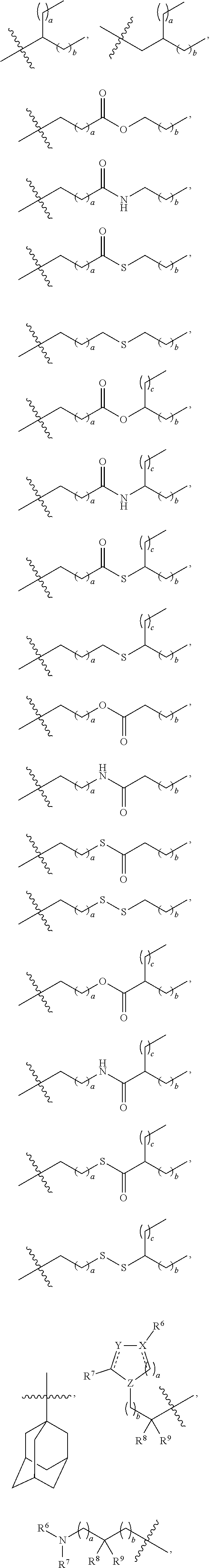
a, b and c are each independently an integer from 0-24;
each of R⁶, R⁷, R⁸ and R⁹ is independently selected from H, C1-C24 alkyl, C1-C24 alkenyl, C1-C24 alkynyl, substituted alkyl, substituted alkenyl, substituted alkynyl, substituted acyl, substituted carbocyclyl, substituted heterocyclyl, substituted aryl, substituted heteroaryl, 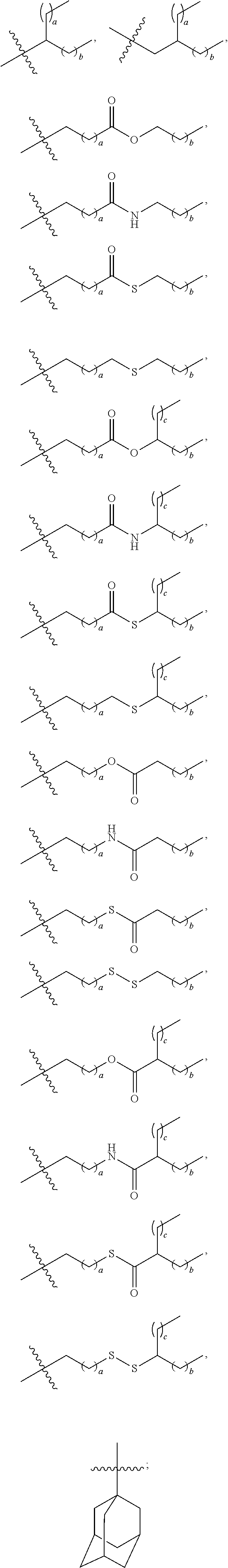
a, b and c are each independently an integer from 0-24;
each X is independently selected from CH or N;
each Y is independently selected from CH₂, NH, O, or S;
each Z is independently selected from CH or N; and
each saccharide is independently selected from monosaccharides, disaccharides, oligosaccharides, and polysaccharides.

12. The composition of claim 7, wherein the LNP comprises a modular lipid compound selected from Formulae X-XX, wherein Formula X is 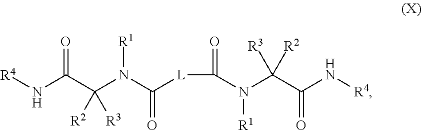
or a salt or isomer thereof;
wherein Formula XI is 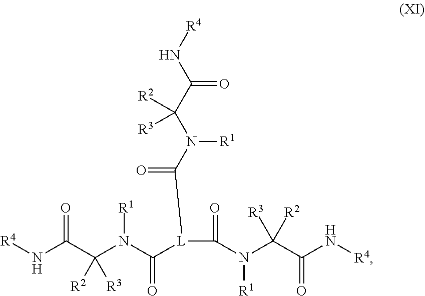
or salt or isomer thereof;
wherein Formula XII is 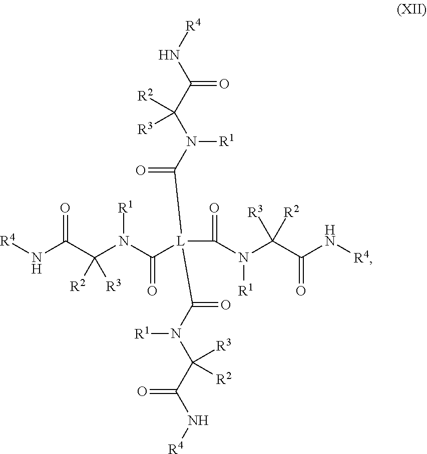
or salt or isomer thereof;
wherein Formula XIII is 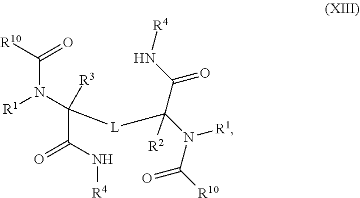
or salt or isomer thereof;
wherein Formula IXX is 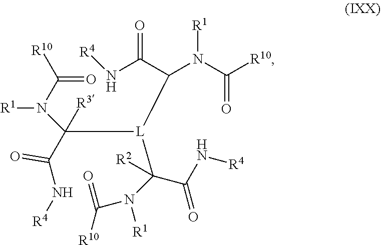
or a salt or isomer thereof,
wherein Formula XX is 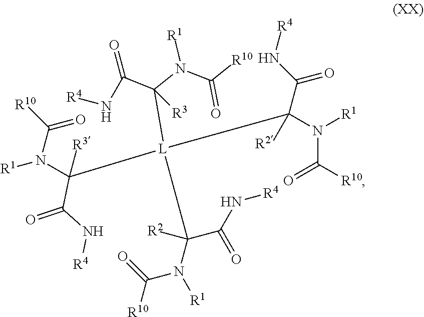
or a salt or isomer thereof;
wherein each R¹, R⁴, and R¹⁰, is independently selected from C₂-C₂₄ alkyl, C₂-C₂₄ alkenyl, C₂-C₂₄ alkynyl, substituted C₂-C₂₄ alkyl, substituted alkenyl, substituted alkynyl, substituted acyl, substituted carbocyclyl, substituted heterocyclyl, substituted aryl, substituted heteroaryl, poly(ethylene glycol) (PEG), 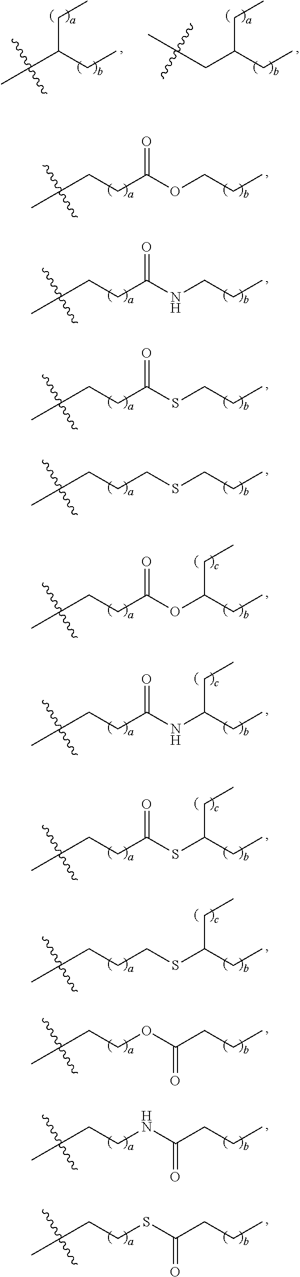 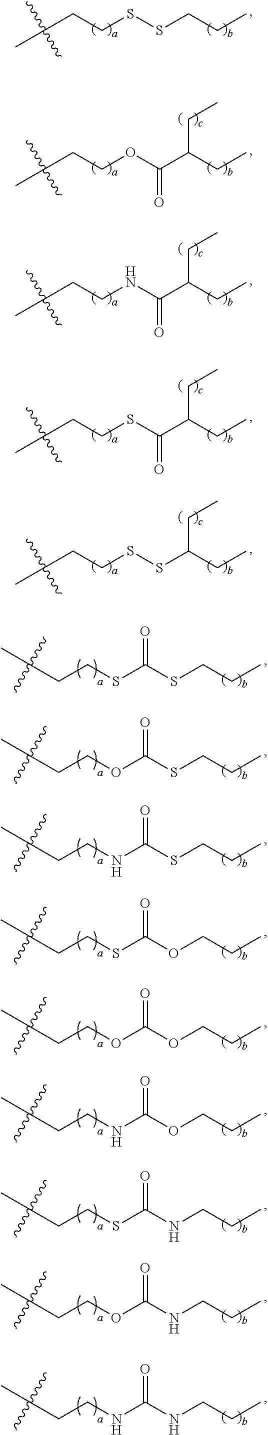 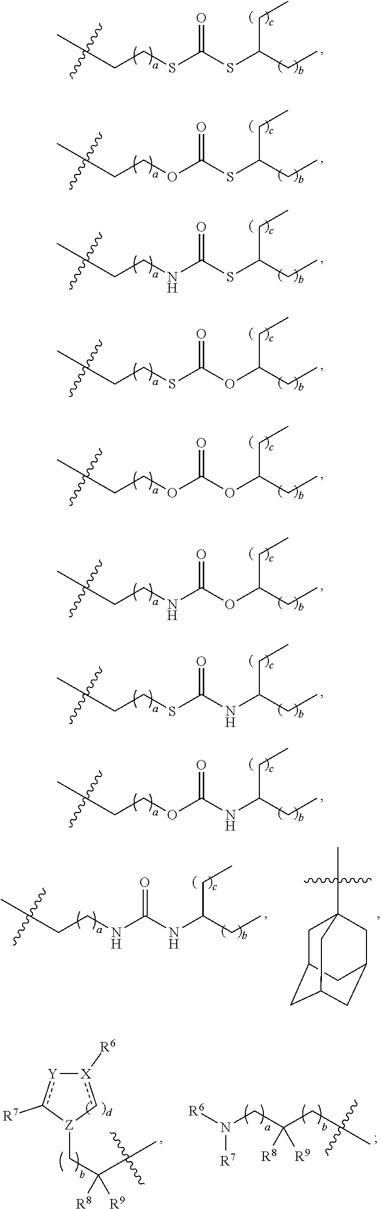
wherein each R², R^{2′}, R³ and R^{3′} is independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, poly(ethylene glycol) (PEG) and 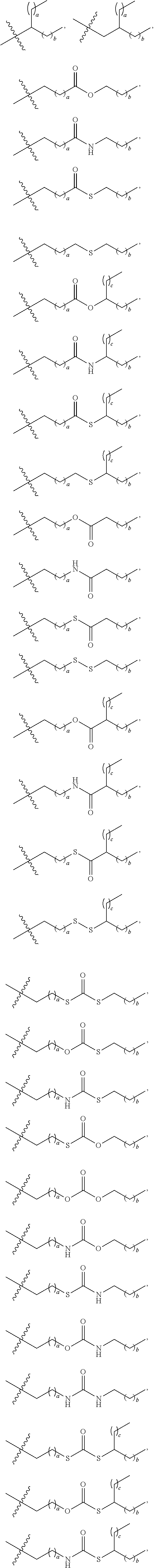 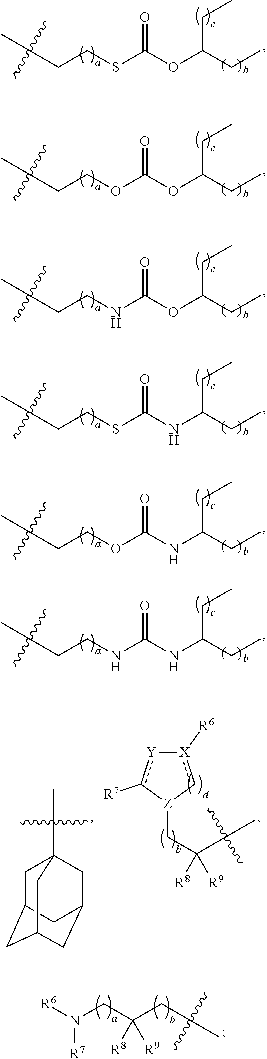
wherein each L is independently selected from alkyl, alkenyl, alkynyl, substituted alkyl, substituted alkenyl, substituted alkynyl, substituted acyl, substituted carbocyclyl, substituted heterocyclyl, substituted aryl, substituted heteroaryl, poly(ethylene glycol) (PEG) and 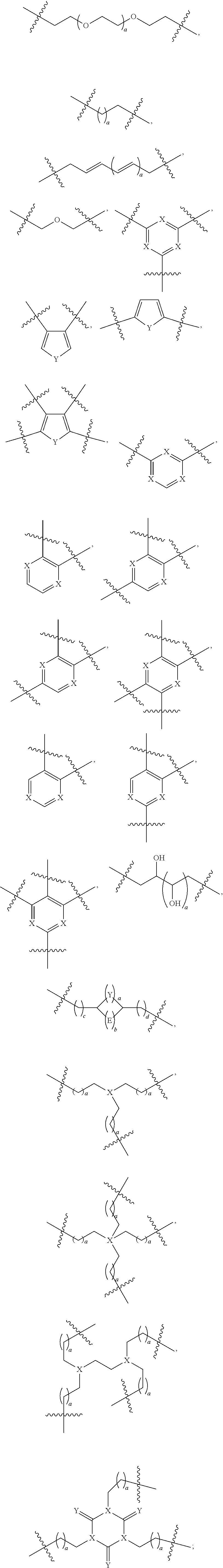
wherein R⁶, R⁷, R⁸ and R⁹ are each independently selected from H, C₁-C₂₄ alkyl, C₁-C₂₄ alkenyl, C₁-C₂₄ alkynyl, substituted alkyl, substituted alkenyl, substituted alkynyl, substituted acyl, substituted carbocyclyl, substituted heterocyclyl, substituted aryl, substituted heteroaryl, substituted carbocyclyl, substituted heterocyclyl, substituted aryl, substituted heteroaryl, 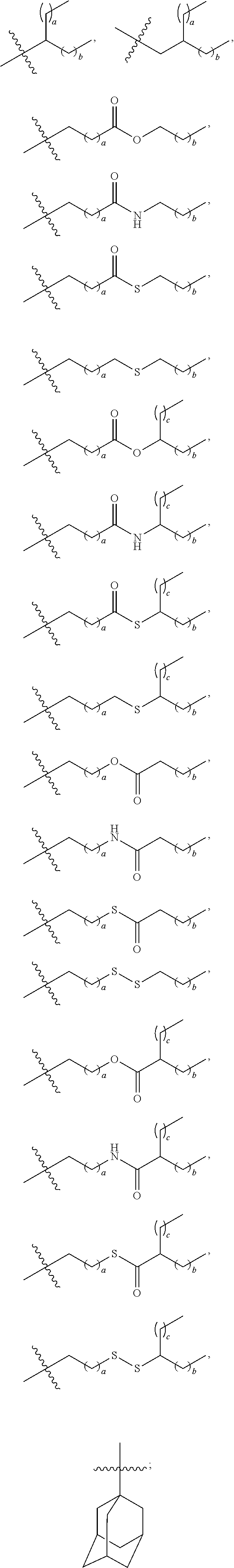
a, b, c and d are each independently an integer from 0-24;
each E is independently selected from CH₂, NH, O, or S;
each X is independently selected from CH or N;
each Y is independently selected from CH₂, NH, O, or S; and
each Z is independently selected from CH or N.

13. The composition of claim 7, wherein the LNP comprises a modular lipid compound, wherein the modular lipid is M1, M2, M3, M4, M5, M6, M7, M8, M9, M10, M11, M12, M13, M14, M15, M16, M17, M18, M19, M20, M21, M22, M23, M24, M25, M26, M27, M28, M29, p1_E2, P1_F11, P1_C6, P1_E8, or a salt or isomer thereof.

14. The composition of claim 1, wherein the first type nucleic acid construct comprises an operably linked nucleic acid sequence comprising from 5′ to 3′:
[5′ UTR]-[nsP 1-4]-[SGP]-[GOI]-[3′ UTR]-[Poly A]
wherein,
5′ UTR is a 5′ untranslated region,
nsP is a plurality of non-structural replicase domain sequences,
SGP is a subgenomic promoter,
GOI is one gene of interest or a plurality of genes of interest,
Y UTR is a 3′ untranslated region, and
Poly A is a poly A tail.

15. The composition of claim 1, wherein the first type nucleic acid construct comprises an operably linked nucleic acid sequence comprising from 5′ to 3′:
[5′ UTR]-[nsP]—[ORF]-[3′ UTR]-[PolyA]
wherein
5′ UTR is a 5′ untranslated region,
nsP is at least one non-structural replicase domain sequence,
ORF is two or more open reading frames,
Y UTR is a 3′ untranslated region, and
Poly-A is a 3′ poly-adenylated tail (poly-A tail),
wherein the ORF comprises at least two SGPs, each operably linked to at least one GOI.

16. The composition of claim 1, wherein the first type nucleic acid construct comprises an operably linked nucleic acid sequence comprising from 5′ to 3′:
[5′ UTR]-[nsP]-[SGP]-[L]-[GOI]-[L]-[3′ UTR]-[PolyA]
wherein
5′ UTR is a 5′ untranslated region,
nsP is a plurality of non-structural replicase domain sequences,
L is a linker independently selected from a sequence comprising any one of SEQ ID NOs: 27-37,
SGP is a subgenomic promoter,
GOI is one or more genes of interest,
3′UTR is a 3′ untranslated region, and
Poly-A is a poly-A tail.

17. The composition of claim 1, wherein the first type nucleic acid construct is a sa-mRNA, wherein the sa-mRNA is SEQ ID NO: 41, 42, 73, 74, 75, or 76 comprising at least one substitution selected from:
a) a substitution of C with A at position 2623;
b) a substitution of T with C at position 3309;
c) a substitution of A with C at position 3493;
d) a substitution of A with G at position 3867;
e) a substitution of A with G at position 4674;
f) a substitution of G with A at position 5795: or
g) a substitution of A with G at position 5897,
wherein position numbers are counted from the first type nucleotide of the sa-mRNA.

18. A pharmaceutical composition comprising the composition of claim 1 and a pharmaceutically acceptable carrier.

19. A method of treating a subject having a tumor or cancer, comprising administering to a subject the pharmaceutical composition of claim 18.

20. A method of inducing apoptosis of a cancerous cell or tumor, comprising contacting the cancerous cell with the pharmaceutical composition of claim 18 such that the first nucleic acid construct and at least one virus structural protein replicate within said cancerous cell, to express the GOI, wherein the first nucleic acid replication, expression of the GOI and the at least one virus structural protein replication within the cancerous cell results in cell death.

21. A method of delivering a GOI encoding a therapeutic gene product to cancerous cells in vivo comprising contacting the cancerous cells with the pharmaceutical composition of claim 18.

22. A method of effecting in vivo synthesis of an oncolytic defective viral particle comprising contacting a cancerous cell with the pharmaceutical composition of claim 18 such that the first nucleic acid construct and the at least one virus structural protein replicate within said cancerous cell and express the GOI.

23. A method of inducing production of 1) a therapeutic payload 2) a pseudoviral particle and 3) sa-mRNA in a cell comprising contacting a cancerous cell with the pharmaceutical composition of claim 18 such that the first type nucleic acid construct and the second type nucleic construct produce the at least one virus structural protein, the sa-mRNA, and the GOI within said cancerous cell, and
wherein the at least one virus structural protein form the pseudoviral particle, which encapsulates the sa-mRNA.

24. A method of increasing expression of a polypeptide encoded by a GOI in a cell comprising contacting the cell with the pharmaceutical composition of claim 18 such that the first nucleic acid construct and the second nucleic construct produce the at least one virus structural protein and the sa-mRNA within said cell, and the first nucleic acid construct expresses the GOI within said cell,
wherein the at least one virus structural protein forms a pseudoviral particle that encapsulates one or more of the sa-mRNA, and
wherein expression of the polypeptide is increased by at least 100-fold compared to contacting the cell with the pharmaceutical composition in the absence of the second nucleic acid construct.

25. A method of increasing expression of a polypeptide encoded by a GOI in a cell comprising contacting the cell with the pharmaceutical composition of claim 18 such that the first nucleic acid construct and the second nucleic construct produce the at least one virus structural protein and the sa-mRNA within said cell, and the first nucleic acid construct expresses the GOI within said cell,
wherein the at least one virus structural protein forms a pseudoviral particle, which encapsulates one or more of the sa-mRNA, and
wherein expression of the polypeptide is increased by 50-fold, 100-fold, 200-fold, 300-fold, 400-fold, 500-fold, or more compared to contacting the cell with the pharmaceutical composition in the absence of the second nucleic acid construct.

26. A method of increasing CD8 T cell in a tumor draining lymph node or spleen comprising contacting a tumor with the pharmaceutical composition of claim 18 such that the first nucleic acid construct and the second nucleic construct produce the at least one virus structural protein and the sa-mRNA within said tumor to produce the at least one virus structural protein and the sa-mRNA, and the first nucleic acid construct expresses the GOI within said tumor,
wherein the at least one virus structural protein forms a pseudoviral particle, which encapsulates one or more of the sa-mRNA, and
wherein CD8 T cell count is increased by 5-fold, 6-fold, 7-fold, 8-fold, 9-fold, 10-fold, or more compared to contacting the cell with the pharmaceutical composition in the absence of the second nucleic acid construct.

27. A method of treating a subject with a payload delivery system encapsulating a mRNA encoding at least one gene of interest (GOI) or a plurality of GOIs and minimizing immunogenicity of the payload delivery system comprising administering the pharmaceutical composition of claim 18 to the subject such that the first nucleic acid construct and the second nucleic construct produce the at least one virus structural protein and the sa-mRNA within a cell of the subject, and the first nucleic acid construct expresses the GOI within said cell,
wherein the at least one virus structural protein forms a pseudoviral particle, which encapsulates one or more of the sa-mRNA, and wherein said non-viral payload delivery system has reduced immunogenicity compared to viral payload delivery systems.

28. A method of producing a mRNA comprising:
a) contacting cells with a nucleic acid template encoding two expression units, the nucleic acid template comprising:
i) an origin of replication sequence (Ori);
ii) a first expression unit encoding a first nucleotide sequence that is operably linked to a first promoter; and
iii) a second expression unit encoding a second nucleotide sequence that is operably linked to a second promoter,
wherein the first expression unit encodes a selectable marker and the second expression unit encodes a mRNA;
b) selecting cells that express the selectable marker;
c) subculturing the selected cells to obtain a population of cells that express the selectable marker;
d) propagating the population of cells to increase the copy number of nucleic acid template replicates comprising the second expression unit encoding the mRNA; and
e) cleaving the nucleic acid template replicates between the first expression unit and the second expression unit to produce the mRNA.

29. A nucleic acid molecule comprising any one or combination of SEQ ID NOs in Table 16.

30. A method of treating a subject having a tumor or cancer, comprising administering to a subject a composition comprising an IL-12 and an IL-18 or IL-12 and an mutant IL-18.


SEQUENCE LISTING (108 sequences)

SEQ ID NO: 1 - DNA, 38 bp, synthetic construct
  source 1..38: mol_type = other DNA
taacctgaatggactacgacatagtctagtccgccaag

SEQ ID NO: 2 - DNA, 92 bp, synthetic construct
  source 1..92: mol_type = other DNA
gaacttccatcatagttatggccatgactactctagctagcagtgttaaatcattcagctacctgagaggggcccctataactctctacggc

SEQ ID NO: 3 - DNA, 30 bp, synthetic construct
  source 1..30: mol_type = other DNA
atggactacgacatagtctagtccgccaag

SEQ ID NO: 4 - DNA, 939 bp, Mus musculus
  source 1..939: mol_type = other DNA
atgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagctcttcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagt

SEQ ID NO: 5 - DNA, 579 bp, Mus musculus
  source 1..579: mol_type = other DNA
agggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcc

SEQ ID NO: 6 - DNA, 426 bp, Mus musculus
  source 1..426: mol_type = other DNA
gggtacctgtcatcagccggcggtagtggtggtgggagccacaagtcctccccccagggtccggatcggctcttgatcagactgagacatctgattgatattgtcgagcagttgaagatctatgagaatgacctcgatcctgagttactgagtgccccacaggacgttaaagggcactgtgaacacgccgcatttgcttgttttcagaaggccaagctgaaaccttctaatcccgggaataacaaaactttcattatcgatctcgtcgcgcagctgaggcggcgacttcctgcacggcggggggggaaaaagcaaaagcacatcgcaaagtgtccctcatgcgactcttacgagaaacgtacccctaaggagttccttgaaagactcaaatggctgctgcaaaagatgatccaccagcatctcagc

SEQ ID NO: 7 - DNA, 918 bp, Homo sapiens
  source 1..918: mol_type = other DNA
atatgggagctgaagaaggacgtgtatgtcgtggagctggactggtacccagatgctcctggcgaaatggtggttttaacatgtgatacccccgaggaggacggcatcacatggactctggaccagagttctgaggtgctggggtccggcaagactctgacaatccaggttaaggagttcggcgacgcaggacagtacacttgtcacaagggaggtgaggtgctttctcacagcctgttgctgctccataagaaggaagacggtatttggtcaaccgacatcctcaaggaccagaaggagcccaaaaacaagacctttctgagatgtgaggccaagaattacagcggtagattcacttgttggtggctcaccaccatatccacagacttgaccttcagtgtcaaaagttcacgagggagctcagatcctcaaggcgttacctgtggcgcagcgacgctgtccgcagaaagagtcaggggagacaacaaggaatacgagtactctgtcgagtgccaggaggattccgcctgtccggccgcagaggagtctttacctattgaggtgatggtcgatgccgtgcacaagcttaagtacgagaattacacatcaagttttttcatccgcgacatcattaaacctgatccaccaaagaacctgcagctcaagcctctgaagaatagcaggcaggtcgaggtaagctgggagtatcctgatacctggtccaccccccacagttatttcagcctcaccttctgcgtccaagtccagggaaagagcaagagagagaagaaggatagggtgttcacagataagacttcagctactgtgatctgcagaaagaatgcgtctatctctgtgcgagcacaagacaggtactacagttctagctggagcgagtgggcatcagtcccctgcagt

SEQ ID NO: 8 - DNA, 591 bp, Homo sapiens
  source 1..591: mol_type = other DNA
aggaacctcccagttgctacacctgacccgggaatgtttccatgcctccaccattcccagaatctcctccgagccgtgtccaatatgctgcaaaaggctcggcagaccttggagttttacccttgcacctcagaagaaatcgatcatgaggatatcacaaaggataagacgagcactgttgaggcatgcctgcccctggagctaactaagaatgagtcttgcctgaacagcagggagacttccttcattaccaacggtagctgtcttgccagcaggaagacatcttttatgatggccctgtgtctatctagcatatatgaagacctgaagatgtaccaggtggaattcaaaaccatgaatgctaagcttctcatggatcccaagaggcaaatcttcctggaccagaatatgcttgctgtcatagatgaactgatgcaggcgttgaattttaacagcgagacggtgcctcaaaaaagctcactggaagaacctgatttttataaaacgaagatcaagctgtgtattttactacacgcctttagaatccgcgctgttaccatcgacagagtaatgtcctacctaaatgcttca

SEQ ID NO: 9 - DNA, 393 bp, Homo sapiens
  source 1..393: mol_type = other DNA
caggacaggcatatgatccggatgcggcagctgatcgatattgtagaccagttgaagaattatgtgaacgacttagtgccggaattcctccccgcccccgaggacgtggagactaattgtgagtggtctgcattctcatgcttccaaaaagcacagctgaagagtgccaataccggcaataacgaaaggatcatcaatgtaagtataaagaagttaaaacgcaaaccgcccagtaccaacgctggacgcaggcaaaaacacaggctgacatgcccctcgtgtgattcgtacgaaaaaaaacctccaaaggaattcctggaaaggttcaagtccttattacagaaaatgattcaccagcacctgagtagtaggacccacggatccgaagactcc

SEQ ID NO: 10 - DNA, 589 bp, synthetic construct
  source 1..589: mol_type = other DNA
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 11 - DNA, 1752 bp, synthetic construct
  source 1..1752: mol_type = other DNA
atgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcg

SEQ ID NO: 12 - DNA, 988 bp, synthetic construct
  source 1..988: mol_type = other DNA
atgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcg

SEQ ID NO: 13 - DNA, 24 bp, synthetic construct
  source 1..24: mol_type = other DNA
taatacgactcactataggatagg

SEQ ID NO: 14 - DNA, 19 bp, Escherichia coli
  source 1..19: mol_type = other DNA
taatacgactcactatagg

SEQ ID NO: 15 - RNA, 39 nt, synthetic construct
  source 1..39: mol_type = other RNA
cggcgcatgagagaagcccagaccaattacctacccaaa

SEQ ID NO: 16 - RNA, 26 nt, synthetic construct
  source 1..26: mol_type = other RNA
ttcgaaggcgcgcctctagagccacc

SEQ ID NO: 17 - RNA, 40 nt, synthetic construct
  source 1..40: mol_type = other RNA
gcggcgcatgagagaagcccagaccaattacctacccaaa

SEQ ID NO: 18 - RNA, 50 nt, synthetic construct
  source 1..50: mol_type = other RNA
acatttgcttctgacacaactgtgttcactagcaacctcaaacagacacc

SEQ ID NO: 19 - RNA, 7487 nt, unidentified
  source 1..7487: mol_type = other RNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctga

SEQ ID NO: 20 - DNA, 57 bp, unidentified
  source 1..57: mol_type = other DNA
atggactggacctggcgaatactgttcttggttgccgccgctacagggactcacgca

SEQ ID NO: 21 - RNA, 138 nt, unidentified
  source 1..138: mol_type = other RNA
catcgatgatatcgcggccgcatacagcagcaattggcaagctgcttacatagaactcgcggcgattggcatgccgccttaaaatttttattttatttttcttttcttttccgaatcggattttgtttttaatatttc

SEQ ID NO: 22 - RNA, 154 nt, unidentified
  source 1..154: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagc

SEQ ID NO: 23 - DNA, 1566 bp, Homo sapiens
  source 1..1566: mol_type = other DNA
atgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagttcctcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagtggaggggggtcagggggtggctcaggcggcggcagtgggggcagcagggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcctag

SEQ ID NO: 24 - DNA, 720 bp, Aequorea victoria
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 25 - DNA, 1653 bp, unidentified
  source 1..1653: mol_type = other DNA
atggaggacgctaaaaatatcaagaagggtcccgctcctttctatccactcgaggacggtaccgctggtgagcaactgcacaaggctatgaaacggtatgctcttgtgccaggcaccatagctttcaccgacgcccacatagaggtggacattacttacgcagagtactttgagatgtctgtccggctggcagaggcaatgaaaagatatggcctcaacacaaaccataggatagtggtttgcagcgagaattctcttcagttttttatgcccgtcctcggggctcttttcatcggcgtggctgtcgcccccgccaacgacatatacaacgagcgagagctgctgaacagcatgggaatcagtcagcctacagtggtttttgtatcaaagaagggcttgcaaaagatccttaacgtacagaaaaagctccctattattcagaagatcattataatggattcaaagaccgactatcagggattccagtcaatgtatactttcgtcacttcccaccttcctcctgggtttaatgaatacgacttcgttcccgaaagtttcgatcgggacaaaactattgcactcatcatgaactcctctgggtccactgggctgcctaaaggtgtcgcacttccccacaggactgcatgtgtaaggttctctcacgcacgagatcccatattcggtaaccaaatcattcctgataccgctatcctctctgtagtgcctttccaccatggattcggcatgttcaccactctgggttacttgatatgtgggttccgcgtggtccttatgtacagatttgaagaggagctctttttgaggagcttgcaggactacaaaatccaaagtgctctcctcgtccccacattgttctcattcttcgccaaatctactctcatcgacaagtacgatttgagcaacttgcacgaaattgcttctgggggggctccactctccaaagaagtcggcgaagccgttgctaaaaggttccacttgccaggcattcgccaggggtatggccttactgagacaacctcagctattctcattacccctgagggtgacgataagcctggtgccgtcggtaaggtagtaccatttttcgaggcaaaggtggtcgacttggataccggaaaaaccctcggtgtgaatcagagaggcgaattgtgtgtcaggggtccaatgatcatgagcggttacgtcaataatcccgaggccactaacgccttgattgataaagatggttggcttcattcaggtgatatagcatactgggatgaggatgaacatttcttcattgtcgataggctcaaaagcctgatcaagtataagggttaccaagtggcccctgccgaactcgaatctatactgctgcaacatccaaacatttttgacgctggcgtggccggactgcccgatgatgatgctggtgaactccctgccgccgttgtcgtcctggaacatggtaagaccatgacagaaaaggagattgtagattacgtggcttcacaagtgactacagctaagaaattgcgcgggggtgtcgttttcgtggacgaggtacccaagggcctcactgggaagctcgacgccagaaaaatccgagaaatcctgattaaagcaaagaagggcggaaagattgcagtgtag

SEQ ID NO: 26 - RNA, 3768 nt, Venezuelan equine encephalitis virus
  source 1..3768: mol_type = other RNA
atgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattga

SEQ ID NO: 27 - DNA, 82 bp, synthetic construct
  source 1..82: mol_type = other DNA
cgcgtgataacgcaggaaagaacatgtgagcaaaaggccagcaaaaggccaggaaccgtaaaaaggccgcgttgctggcgtt

SEQ ID NO: 28 - DNA, 31 bp, synthetic construct
  source 1..31: mol_type = other DNA
cacatttccccgaaaagtgccacctgagctc

SEQ ID NO: 29 - DNA, 26 bp, synthetic construct
  source 1..26: mol_type = other DNA
ttcgaaggcgcgcctctagagccacc

SEQ ID NO: 30 - DNA, 31 bp, synthetic construct
  source 1..31: mol_type = other DNA
catcgatgatatcgcggccgcatacagcagc

SEQ ID NO: 31 - DNA, 45 bp, synthetic construct
  source 1..45: mol_type = other DNA
ggaggggggtcagggggtggctcaggcggcggcagtgggggcagc

SEQ ID NO: 32 - DNA, 21 bp, synthetic construct
  source 1..21: mol_type = other DNA
ggcggtagtggtggtgggagc

SEQ ID NO: 33 - DNA, 45 bp, synthetic construct
  source 1..45: mol_type = other DNA
ggtggcggaagcggagggggcagcggaggtgggagcggagggagc

SEQ ID NO: 34 - DNA, 21 bp, synthetic construct
  source 1..21: mol_type = other DNA
ggagggtcaggaggaggatcc

SEQ ID NO: 35 - DNA, 148 bp, synthetic construct
  source 1..148: mol_type = other DNA
gaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatat

SEQ ID NO: 36 - DNA, 11 bp, synthetic construct
  source 1..11: mol_type = other DNA
cgcggaagagc

SEQ ID NO: 37 - DNA, 250 bp, synthetic construct
  source 1..250: mol_type = other DNA
cgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtt

SEQ ID NO: 38 - DNA, 12006 bp, synthetic construct
  source 1..12006: mol_type = other DNA
cttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagttcctcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagtggaggggggtcagggggtggctcaggcggcggcagtgggggcagcagggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcga

SEQ ID NO: 39 - DNA, 3927 bp, synthetic construct
  source 1..3927: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggacatttgcttctgacacaactgtgttcactagcaacctcaaacagacaccatggaggacgctaaaaatatcaagaagggtcccgctcctttctatccactcgaggacggtaccgctggtgagcaactgcacaaggctatgaaacggtatgctcttgtgccaggcaccatagctttcaccgacgcccacatagaggtggacattacttacgcagagtactttgagatgtctgtccggctggcagaggcaatgaaaagatatggcctcaacacaaaccataggatagtggtttgcagcgagaattctcttcagttttttatgcccgtcctcggggctcttttcatcggcgtggctgtcgcccccgccaacgacatatacaacgagcgagagctgctgaacagcatgggaatcagtcagcctacagtggtttttgtatcaaagaagggcttgcaaaagatccttaacgtacagaaaaagctccctattattcagaagatcattataatggattcaaagaccgactatcagggattccagtcaatgtatactttcgtcacttcccaccttcctcctgggtttaatgaatacgacttcgttcccgaaagtttcgatcgggacaaaactattgcactcatcatgaactcctctgggtccactgggctgcctaaaggtgtcgcacttccccacaggactgcatgtgtaaggttctctcacgcacgagatcccatattcggtaaccaaatcattcctgataccgctatcctctctgtagtgcctttccaccatggattcggcatgttcaccactctgggttacttgatatgtgggttccgcgtggtccttatgtacagatttgaagaggagctctttttgaggagcttgcaggactacaaaatccaaagtgctctcctcgtccccacattgttctcattcttcgccaaatctactctcatcgacaagtacgatttgagcaacttgcacgaaattgcttctgggggggctccactctccaaagaagtcggcgaagccgttgctaaaaggttccacttgccaggcattcgccaggggtatggccttactgagacaacctcagctattctcattacccctgagggtgacgataagcctggtgccgtcggtaaggtagtaccatttttcgaggcaaaggtggtcgacttggataccggaaaaaccctcggtgtgaatcagagaggcgaattgtgtgtcaggggtccaatgatcatgagcggttacgtcaataatcccgaggccactaacgccttgattgataaagatggttggcttcattcaggtgatatagcatactgggatgaggatgaacatttcttcattgtcgataggctcaaaagcctgatcaagtataagggttaccaagtggcccctgccgaactcgaatctatactgctgcaacatccaaacatttttgacgctggcgtggccggactgcccgatgatgatgctggtgaactccctgccgccgttgtcgtcctggaacatggtaagaccatgacagaaaaggagattgtagattacgtggcttcacaagtgactacagctaagaaattgcgcgggggtgtcgttttcgtggacgaggtacccaagggcctcactgggaagctcgacgccagaaaaatccgagaaatcctgattaaagcaaagaagggcggaaagattgcagtgtag

SEQ ID NO: 40 - DNA, 6042 bp, synthetic construct
  source 1..6042: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggacatttgcttctgacacaactgtgttcactagcaacctcaaacagacaccatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattga

SEQ ID NO: 41 - RNA, 9380 nt, synthetic construct
  source 1..9380: mol_type = other RNA
cggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagttcctcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagtggaggggggtcagggggtggctcaggcggcggcagtgggggcagcagggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcggaagagc

SEQ ID NO: 42 - RNA, 8467 nt, synthetic construct
  source 1..8467: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 43 - RNA, 1905 nt, synthetic construct
  source 1..1905: mol_type = other RNA
acatttgcttctgacacaactgtgttcactagcaacctcaaacagacaccatggaggacgctaaaaatatcaagaagggtcccgctcctttctatccactcgaggacggtaccgctggtgagcaactgcacaaggctatgaaacggtatgctcttgtgccaggcaccatagctttcaccgacgcccacatagaggtggacattacttacgcagagtactttgagatgtctgtccggctggcagaggcaatgaaaagatatggcctcaacacaaaccataggatagtggtttgcagcgagaattctcttcagttttttatgcccgtcctcggggctcttttcatcggcgtggctgtcgcccccgccaacgacatatacaacgagcgagagctgctgaacagcatgggaatcagtcagcctacagtggtttttgtatcaaagaagggcttgcaaaagatccttaacgtacagaaaaagctccctattattcagaagatcattataatggattcaaagaccgactatcagggattccagtcaatgtatactttcgtcacttcccaccttcctcctgggtttaatgaatacgacttcgttcccgaaagtttcgatcgggacaaaactattgcactcatcatgaactcctctgggtccactgggctgcctaaaggtgtcgcacttccccacaggactgcatgtgtaaggttctctcacgcacgagatcccatattcggtaaccaaatcattcctgataccgctatcctctctgtagtgcctttccaccatggattcggcatgttcaccactctgggttacttgatatgtgggttccgcgtggtccttatgtacagatttgaagaggagctctttttgaggagcttgcaggactacaaaatccaaagtgctctcctcgtccccacattgttctcattcttcgccaaatctactctcatcgacaagtacgatttgagcaacttgcacgaaattgcttctgggggggctccactctccaaagaagtcggcgaagccgttgctaaaaggttccacttgccaggcattcgccaggggtatggccttactgagacaacctcagctattctcattacccctgagggtgacgataagcctggtgccgtcggtaaggtagtaccatttttcgaggcaaaggtggtcgacttggataccggaaaaaccctcggtgtgaatcagagaggcgaattgtgtgtcaggggtccaatgatcatgagcggttacgtcaataatcccgaggccactaacgccttgattgataaagatggttggcttcattcaggtgatatagcatactgggatgaggatgaacatttcttcattgtcgataggctcaaaagcctgatcaagtataagggttaccaagtggcccctgccgaactcgaatctatactgctgcaacatccaaacatttttgacgctggcgtggccggactgcccgatgatgatgctggtgaactccctgccgccgttgtcgtcctggaacatggtaagaccatgacagaaaaggagattgtagattacgtggcttcacaagtgactacagctaagaaattgcgcgggggtgtcgttttcgtggacgaggtacccaagggcctcactgggaagctcgacgccagaaaaatccgagaaatcctgattaaagcaaagaagggcggaaagattgcagtgtaggctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaaaaaaaaaaaaaaaaaaaa

SEQ ID NO: 44 - RNA, 1614 nt, Homo sapiens
  source 1..1614: mol_type = mRNA
atggactggacctggcgaatactgttcttggttgccgccgctacagggactcacgcaatatgggagctgaagaaggacgtgtatgtcgtggagctggactggtacccagatgctcctggcgaaatggtggttttaacatgtgatacccccgaggaggacggcatcacatggactctggaccagagttctgaggtgctggggtccggcaagactctgacaatccaggttaaggagttcggcgacgcaggacagtacacttgtcacaagggaggtgaggtgctttctcacagcctgttgctgctccataagaaggaagacggtatttggtcaaccgacatcctcaaggaccagaaggagcccaaaaacaagacctttctgagatgtgaggccaagaattacagcggtagattcacttgttggtggctcaccaccatatccacagacttgaccttcagtgtcaaaagttcacgagggagctcagatcctcaaggcgttacctgtggcgcagcgacgctgtccgcagaaagagtcaggggagacaacaaggaatacgagtactctgtcgagtgccaggaggattccgcctgtccggccgcagaggagtctttacctattgaggtgatggtcgatgccgtgcacaagcttaagtacgagaattacacatcaagttttttcatccgcgacatcattaaacctgatccaccaaagaacctgcagctcaagcctctgaagaatagcaggcaggtcgaggtaagctgggagtatcctgatacctggtccaccccccacagttatttcagcctcaccttctgcgtccaagtccagggaaagagcaagagagagaagaaggatagggtgttcacagataagacttcagctactgtgatctgcagaaagaatgcgtctatctctgtgcgagcacaagacaggtactacagttctagctggagcgagtgggcatcagtcccctgcagtggtggcggaagcggagggggcagcggaggtgggagcggagggagcaggaacctcccagttgctacacctgacccgggaatgtttccatgcctccaccattcccagaatctcctccgagccgtgtccaatatgctgcaaaaggctcggcagaccttggagttttacccttgcacctcagaagaaatcgatcatgaggatatcacaaaggataagacgagcactgttgaggcatgcctgcccctggagctaactaagaatgagtcttgcctgaacagcagggagacttccttcattaccaacggtagctgtcttgccagcaggaagacatcttttatgatggccctgtgtctatctagcatatatgaagacctgaagatgtaccaggtggaattcaaaaccatgaatgctaagcttctcatggatcccaagaggcaaatcttcctggaccagaatatgcttgctgtcatagatgaactgatgcaggcgttgaattttaacagcgagacggtgcctcaaaaaagctcactggaagaacctgatttttataaaacgaagatcaagctgtgtattttactacacgcctttagaatccgcgctgttaccatcgacagagtaatgtcctacctaaatgcttcatag

SEQ ID NO: 45 - DNA, 462 bp, Homo sapiens
  source 1..462: mol_type = other DNA
atgtaccgcatgcagttgctgtcttgtattgcgcttagcctcgcgttggtcacaaacagtgccccgacctccagttctaccaagaagacgcaactccagcttgagcatcttctcctggacttgcagatgatattgaatggtatcaacaattacaagaatcccaaactcaccaggatgcttacttttaagttttatatgcctaagaaagcgacggaactgaaacaccttcaatgcttggaggaagaactgaaaccgcttgaggaggtactgaatttggcacagtcaaaaaacttccacctgaggccaagggacttgatcagtaacataaacgtaatcgttttggagcttaaggggagcgaaaccacctttatgtgcgagtacgcagacgagacggccacgattgtagaatttttgaatcgctggattactttttgccaaagtatcatcagtacactcacctag

SEQ ID NO: 46 - DNA, 510 bp, Mus musculus
  source 1..510: mol_type = other DNA
atgtattcaatgcagcttgccagttgtgtcacactcactcttgtcctgctggttaattccgccccaacttcaagccccaccagctcccccacctcaagttcaaccgctgaagcacagcaacagcaacaacaacagcaacacttggaacaactcctcatggatctccaggaactgctttctcgaatggagaactaccggaatctgaaacttcccagaatgctgaccttcaagttctacctccctaagcaggctactgaacttaaagatttgcaatgtcttgaagatgaactgggcccacttcgacacgtattggatctgacccagtcaaaatcctttcaattggaggacgcagagaacttcataagcaacatcagggtcaccgtggtgaagctcaaggggtctgacaatacatttgaatgtcaatttgatgatgagtcagccaccgtcgtcgattttttgcggagatggatcgcattctgccagagcataatttctaccagccctcagtag

SEQ ID NO: 47 - DNA, 405 bp, Homo sapiens
  source 1..405: mol_type = other DNA
atgtatcgaatgcaattgctgtcctgcattgcgctctctctggcgcttgtcaccaattcaaactgggttaacgtaatatctgatttgaaaaaaatcgaagacctcatacagagtatgcacatagatgctacgctgtacacagaaagcgacgtacacccgagctgtaaggtcactgccatgaagtgctttcttctcgaactccaagttatcagtctcgaatccggggatgcgtcaatccatgacactgttgagaatctcattattctcgcgaacaactcactcagctcaaatgggaacgtcactgaatccggctgcaaggaatgcgaggaattggaagagaagaatattaaagagttcctgcagagtttcgttcatattgttcaaatgtttatcaacacaagttag

SEQ ID NO: 48 - DNA, 405 bp, Mus musculus
  source 1..405: mol_type = other DNA
atgtattcaatgcagcttgccagttgtgtcacactcactcttgtcctgctggttaattccaattggatcgatgtccggtatgatttggagaagatcgaatctctcatccaaagcatacacattgacaccactttgtatactgatagcgattttcacccatcttgtaaggtgaccgccatgaattgctttctgttggaacttcaagttatacttcacgagtattcaaacatgacactgaatgaaactgtccgaaacgtactctaccttgctaactccaccctttcatctaataagaacgtagctgagagcggatgtaaggagtgtgaggagctcgaagagaagacatttactgaatttcttcagagttttatccgcatagtgcaaatgtttattaacacctcctag

SEQ ID NO: 49 - DNA, 660 bp, Homo sapiens
  source 1..660: mol_type = other DNA
atgtatcgaatgcaattgctgtcctgcattgcgctctctctggcgcttgtcaccaattcaaactgggttaacgtaatatctgatttgaaaaaaatcgaagacctcatacagagtatgcacatagatgctacgctgtacacagaaagcgacgtacacccgagctgtaaggtcactgccatgaagtgctttcttctcgaactccaagttatcagtctcgaatccggggatgcgtcaatccatgacactgttgagaatctcattattctcgcgaacaactcactcagctcaaatgggaacgtcactgaatccggctgcaaggaatgcgaggaattggaagagaagaatattaaagagttcctgcagagtttcgttcatattgttcaaatgtttatcaacacaagtggaggggggtcagggggtggctcaggcggcggcagtgggggcagcataacttgtccacctccgatgagtgtggaacatgctgacatttgggtaaagagttactcactctattcacgagagcgatacatctgtaatagtgggtttaaaagaaaagccggtacatcctctcttactgaatgcgtactcaataaggcgaccaacgtcgcgcactggaccactccttccttgaaatgcatccgcgacccggctctcgtttag

SEQ ID NO: 50 - DNA, 660 bp, Homo sapiens
  source 1..660: mol_type = other DNA
atgtaccgcatgcaacttttgtcttgcattgcacttagcttggcactcgtcactaattccattacatgccctccgcctatgtctgtggagcacgcagacatctgggtaaagtcttactccctctactcaagagaacgatatatctgtaattcagggtttaagcgaaaagcaggaacctcttcactgacagaatgcgtgcttaataaggcgacgaacgttgctcattggactacacctagtttgaagtgtattcgggatccggcactcgtaggaggggggtcagggggtggctcaggcggcggcagtgggggcagcaattgggtaaacgtaattagcgatctgaagaagatcgaggacttgatacaaagtatgcatatcgatgctactctttataccgagtctgatgtacacccctcttgtaaagtaacagcgatgaagtgctttttgttggagttgcaagtgataagcctggaaagtggggatgcgtccatccacgataccgtcgagaacttgataatactggccaacaacagtctctctagtaacggtaacgtaaccgagagcgggtgtaaagaatgcgaagaattggaagagaaaaatatcaaagaattccttcagtcatttgtgcatatagtgcagatgtttatcaacacatcctag

SEQ ID NO: 51 - DNA, 663 bp, Mus musculus
  source 1..663: mol_type = other DNA
atgtattcaatgcagcttgccagttgtgtcacactcactcttgtcctgctggttaattccaattggatcgatgtccggtatgatttggagaagatcgaatctctcatccaaagcatacacattgacaccactttgtatactgatagcgattttcacccatcttgtaaggtgaccgccatgaattgctttctgttggaacttcaagttatacttcacgagtattcaaacatgacactgaatgaaactgtccgaaacgtactctaccttgctaactccaccctttcatctaataagaacgtagctgagagcggatgtaaggagtgtgaggagctcgaagagaagacatttactgaatttcttcagagttttatccgcatagtgcaaatgtttattaacacctccggaggggggtcagggggtggctcaggcggcggcagtgggggcagcggaactacatgtccacctcccgtcagtatcgaacatgcagatatcagagttaaaaattattcagtgaacagcagggagcgctacgtgtgtaatagtggttttaaaaggaaagccggcacttctacattgattgaatgcgttatcaataagaatactaacgtagctcactggaccacaccctctctgaagtgtatccgagacccttcacttgcctag

SEQ ID NO: 52 - DNA, 663 bp, Mus musculus
  source 1..663: mol_type = other DNA
atgtattcaatgcagcttgccagttgtgtcacactcactcttgtcctgctggttaattccggaactacttgcccaccacccgtcagtatcgagcacgctgatataagagttaaaaactattctgtaaattctagggaaagatatgtatgtaacagcggtttcaaaaggaaggcagggacatcaacccttatcgagtgcgtcattaacaaaaatacaaacgtcgcccattggaccactccatcactcaaatgtatacgagaccctagcttggcaggaggggggtcagggggtggctcaggcggcggcagtgggggcagcaattggatagatgtccggtacgacctcgagaagatcgagtcacttattcaatctatacatattgacactaccctgtatactgacagcgactttcatccaagttgtaaagtcactgctatgaattgctttttgctcgagttgcaggtcatcctgcatgagtattctaatatgactttgaatgaaaccgtcagaaatgtcttgtatcttgctaacagtacccttagctccaacaagaatgttgccgaatcaggttgtaaagaatgcgaggagttggaagagaaaacctttaccgaatttcttcaatcattcatcagaattgtacaaatgttcatcaatacttcatag

SEQ ID NO: 53 - DNA, 540 bp, Mus musculus
  source 1..540: mol_type = other DNA
atgacctcccggcttgtgagggtactggctgctgctatgctggtggctgctgctgtgagtgtggcaaactttgggagactccactgtaccactgccgttatacggaatataaacgatcaggtcctctttgtagacaagagacagccagttttcgaggacatgactgatatagatcagagtgcctctgagcctcaaactcggctcataatatacatgtataaggatagcgaggttcggggacttgccgtgactttgtccgtgaaagactcaaaaatgagcaccctgtcatgcaaaaataagataatttctttcgaggaaatggaccctcccgagaacattgatgatatccagtccgacttgatatttttccagaaacgggttcccggtcataacaaaatggagtttgagtcttcattgtacgagggtcattttttggcatgccagaaggaggatgatgccttcaaactgattttgaagaaaaaagacgagaacggagacaagtctgtcatgtttacacttaccaaccttcatcaatcctag

SEQ ID NO: 54 - DNA, 540 bp, Mus musculus
  source 1..540: mol_type = other DNA
atgacctcccggcttgtgagggtactggctgctgctatgctggtggctgctgctgtgagtgtggcacacttcggacgactccattgtactacagccgttatacgaaatatcaatgatcaggtgttgtttgtagacaaaaggcagcccgtgtttgaagacatgactgatattgatcagagtgcctctgagccccagactagactgattatatatgcttatggcgactctagagctagagggaaagcagtaactctgtcagtgaaagacagcaagatgtctaccctgtcttgcaagaataaaataatctcatttgaggagatggacccccctgaaaacatagatgacatccaaagtgacctgatattctttcagaagcgggtacccggtcataacaaaatggagttcgagtcaagcctctatgagggtcactttctcgcctgccaaaaggaagatgacgccttcaaattgatcttgaaaaaaaaagacgagaatggtgataagtctgtcatgttcacactcactaatttgcatcagtcatag

SEQ ID NO: 55 - DNA, 1257 bp, Mus musculus
  source 1..1257: mol_type = other DNA
atgaatccatccgcagctgtaattttctgccttatcctcctggggctgagtggaacccaaggaatccccctcgctcgaacagtacgctgcaactgtatccacatagacgatggccccgttcgaatgagggctatcggaaagctcgaaataatacccgcctctctctcatgcccccgcgtagaaataatagcaactatgaagaagaacgacgagcaacgatgcttgaacccagaaagcaagactatcaagaacttgatgaaagcttttagccaaaagcggagtaaacgagcccccgggggaggcggctccggcggaggagggagtggtggcggtggatccggaggcggtgggagtacccttgtgattaggaatgcacgatgcagctgtataagcacctcccggggcactattcactataagagccttaaagatctcaagcagtttgcaccatctccaaattgtaacaaaactgagattattgctacactgaagaacggggaccaaacctgcctggacccagatagtgccaatgttaaaaagcttatgaaggaatgggagaaaaaaatcagtcaaaagaaaaagcaaaaacgcggtaaaaaacaccagaaaaacatgaagaatcggaaaccaaaaaccccacagtctaggagaaggagtcgaaaaacaacagggggaggcggaagtggcggcgggggcagcggtggcgggggatcaggcggcgggggcagtttccttatgttcaaacaagggcggtgcctgtgtatagggccaggtatgaaagcagtgaagatggctgaaatcgaaaaagcaagtgtgatctaccctagtaatggctgtgataaagttgaagttattgtgactatgaaagctcacaagagacagcggtgtttggatcctagatccaaacaagcccgactcatcatgcaagccatagaaaagaagaacttcctgcgaaggcagaatatgggtggaggtggaagcggtggcgggggtagcggcgggggaggtagtggtgggggcggtagcgtaactagtgctggaccagaggagtccgatggggatctctcttgtgtctgcgttaagaccatatcatccgggattcaccttaagcacattaccagcctcgaagtgatcaaagctggtaggcattgtgccgtgccccagctcatagccacattgaagaatgggaggaaaatttgccttgatagacaggcaccattgtataagaaagtcattaaaaaaattcttgaatcatag

SEQ ID NO: 56 - RNA, 3765 nt, Semliki Forest virus
  source 1..3765: mol_type = other RNA
atgaattacatccctacgcaaacgttttacgggcgcagatggcgacctcgacccgccgctcgcccctggcctttgcaagctacgcccgtagcccccgtggtcccagactttcaggcacagcaaatgcagcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaaggggagggtagtcgctatcgtcctgggcggggccaacgagggctcacgcacagcactgtcggtggtcacctggaacaaagatatggtgactagagtgacccccgaggggtccgaagagtggtccgccccgctgattactgccatgtgtgtccttgccaatgctaccttcccgtgcttccagcccccgtgtgtaccttgctgctatgaaaacaacgcagaggccacactacggatgctcgaggataacgtggataggccagggtactacgacctccttcaggcagccttgacgtgccgaaacggaacaagacaccggcgcagcgtgtcgcaacacttcaacgtgtataaggctacacgcccttacatcgcgtactgcgccgactgcggagcagggcactcgtgtcatagccccgtagcaattgaagcggtcaggtccgaagctaccgacgggatgctgaagattcagttctcggcacaaattggcatagataagagtgacaatcatgactacacgaagataaggtacgcagacgggcacgccattgagaatgccgtccggtcatctttgaaggtagccacctccggagactgtttcgtccatggcacaatgggacatttcatactggcaaagtgcccaccgggtgaattcctgcaggtctcgatccaggacaccagaaacgcggtccgtgcctgcagaatacaatatcatcatgaccctcaaccggtgggtagagaaaaatttacaattagaccacactatggaaaagagatcccttgcaccacttatcaacagaccacagcgaagaccgtggaggaaatcgacatgcatatgccgccagatacgccggacaggacgttgctatcacagcaatctggcaatgtaaagatcacagtcggaggaaagaaggtgaaatacaactgcacctgtggaaccggaaacgttggcactactaattcggacatgacgatcaacacgtgtctaatagagcagtgccacgtctcagtgacggaccataagaaatggcagttcaactcacctttcgtcccgagagccgacgaaccggctagaaaaggcaaagtccatatcccattcccgttggacaacatcacatgcagagttccaatggcgcgcgaaccaaccgtcatccacggcaaaagagaagtgacactgcaccttcacccagatcatcccacgctcttttcctaccgcacactgggtgaggacccgcagtatcacgaggaatgggtgacagcggcggtggaacggaccatacccgtaccagtggacgggatggagtaccactggggaaacaacgacccagtgaggctttggtctcaactcaccactgaagggaaaccgcacggctggccgcatcagatcgtacagtactactatgggctttacccggccgctacagtatccgcggtcgtcgggatgagcttactggcgttgatatcgatcttcgcgtcgtgctacatgctggttgcggcccgcagtaagtgcttgaccccttatgctttaacaccaggagctgcagttccgtggacgctggggatactctgctgcgccccgcgggcgcacgcagctagtgtggcagagactatggcctacttgtgggaccaaaaccaagcgttgttctggttggagtttgcggcccctgttgcctgcatcctcatcatcacgtattgcctcagaaacgtgctgtgttgctgtaagagcctttcttttttagtgctactgagcctcggggcaaccgccagagcttacgaacattcgacagtaatgccgaacgtggtggggttcccgtataaggctcacattgaaaggccaggatatagccccctcactttgcagatgcaggttgttgaaaccagcctcgaaccaacccttaatttggaatacataacctgtgagtacaagacggtcgtcccgtcgccgtacgtgaagtgctgcggcgcctcagagtgctccactaaagagaagcctgactaccaatgcaaggtttacacaggcgtgtacccgttcatgtggggaggggcatattgcttctgcgactcagaaaacacgcaactcagcgaggcgtacgtcgatcgatcggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccgacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataa

SEQ ID NO: 57 - RNA, 3738 nt, Sindbis virus
  source 1..3738: mol_type = other RNA
atgaatagaggaatatttaacatgctgagccgccgccccttccccgcccccacagctatgtggaggcctaggcgaaggaggcaggcggcaccactgcccgcccgtaatgggttggcatcccaaattcaacagctaaccacagctgtcagtgccctagtcattggtcaagccacccgacctcaacaaccaaggccacgcccgctaccacgccctaagaagcagtccccgaagcaaccaccgaatgctaaaaagacaaaaccgcaggagaacaaggagaaacaacccgcaaaaccgaagccgggtaagagacagcgtatggcactaaaattgatgctgacaggactctttgacgtgaacaacgaagccggtgacgtaataggacacgccctcgctatggaaggcaaagtgatgaagccacttcacgtgaaaggggtcatagaccacccggttttggcaaagctgaaatttaccaagtcatcagcgtacgacatggagttcgcccagcttccagccagtatgaaaagcgaagcatttgcatacaccagcgagcaccctgaaggcttctacaactggcatcatggtgccgtgcagtacagcggcggtcgctttacagtcccgcgcggagttgggggaaaaggcgacagtggaagaccgatcatggataatacaggtaaggtggtcgcgatagtgctgggcggcgctgacgaaggcgcccgcactgctctctccgtcgtcacttggaacagcaaaggaaagaccatcaaaacgactccggaagggactgaggaatggtctgcagcaccattagtcacggctatgtgcttctttgagaatgtatcgttcccgtgcagtcgtccgcctacgtgctattcccgaaaaccatctcgtgcccttgacatcctggaggagaacgtgaacaacgacgcctacgataccttattagcggcagtgttgaagtgcaggacgtccggtcggaacaaacgcagcatcaccgacgatttcaccctgaccagcccgtacctgggcacttgctcgtattgccatcatactgaaccctgcttcagcccaattaagatcgagcaagtttgggacgaggcggacgacggatcaatacgaatacagacatccgcccagttcgggtacgaccaaagtggggcagcagacgtcaacaagtacaggtacatgtcaattgaccaggaccatgccgtgaaagaaggctctatggatcatatcaagatcagcacatccgggccctgtaggcggcttcaatcacaaaggtacttcttgcttgcgaaatgcccaccgggagatagcgttacggttagcataaccggcggcactatggccacctcctgtacattagcacgtaaaataaggcaaaaattcgtaggacgtgagaagtatgaccttcccccagcacacggaaagaacatcccctgcaccgtgtatgatcgtcttaaggagacgtcagccggatacatcaccatgcacaggcctggacctcatgcctatacctcatacctagaggctacctcaggaaaaatttacgccaaaccaccatcaggaaagaacatcacgtacgagtgtaagtgtggcgattataaaactgcgaccgtttccgtcagaacagagatcgcaggatgtactgccataaaacagtgcgtggcgtacaagagcgaccagaccaagtgggtgttcaactcgcccgatctaatcaggcacgctgaccacacagcccaaggtaaattccactttcctttcaagcctaccttcagtacatgcctggtaccactggcacacgagccgactgtaattcatgggttcaaacacataagcctccatttagacacagaccacccaaccttgctcaccattagaagactgggcgagaaacccgaaccgacgtcggagtggatctcaggaaaaacagttaggaattttactgtcgacagagacgggctagagtatatatggggcaatcacgaaccggtgagagtatatgcacaagagtccgcccccggtgaccctcacggatggccacacaaaataattcaacactactatcaacgccacccagtatacactagcttggctgtagcagcttccgctgtggcggtgatagtcggcctggccaccgccgtgatgtgtatctgcagagcgcgacgggagtgcctaaccccgtacgcgttagccccgaatgcagtcatcccgacgtccttggccttattgtgctgcgttcggtccgccaacgccgaatctttcaccgacacgatgggatacctatggtcgaacagtcaaacatttttctgggtgcagctgtgcatccccttggcagtagtaatagtactcgtacgctgctgttcgtgctgcctcccttttttagtggttgcaggcgcctacctggcgaaggtagacgccttcgaacatgcgaccactgtcccaaatgtgccgaagattccgtataaagctttggtcgagagagccggatacgccccgcttaacttggaaattactgttatgtcttcagaggtcctcccttcgattcaccaagaatacatcacctgcaaatactccactatcgtgccttccccgaaagtgaaatgctgcggttcccttgagtgccaacccgcttcgcatgccgactacaactgcaaagtatttggcggcgtttaccctttcatgtggggtggagcgcaatgtttttgcgacagcgagaacagccagatgagtgaggcgtatgtcgagctatcggccgattgtgcccttgatcacgcacaggcggtgaaagtgcacacagccgcaatgaaggtcggactacgcattgtgtatgggaacaccactagcctcctggatgcgtacgtaaacggagtcacgccaggcacatctaaagacctgaaagtcattgccggacccatctcgtccgcgttttccccttttgactctaaagtcgtgatacaccgagggctcgtgtacaattacgattttccagagtacggggcaatgaaaccgggtgcctttggggacattcaagctagctctttgacgggcaacgacctgattgccagcaccgacatacgtttgcttaaacccgcagcaaagaatgtgcacgttccctatacccaagcagcatcaggatttgaaatttggaagaacaactccggaagaccactgcaagagacggccccatttggctgtaagattgccgtcaaccctctgaaggccgtggattgttcctatgggaacatcccgatatcgattgatatcccccacgccgcctttattagaccatccgatgcgccatttatctccgaagtgaagtgcgaagtcagcgactgcacatactctgctgacttcggaggcatggccaccctacagtatgtgtctgaccgagagggccagtgcccagtgcattcccattcaagtaccgccacactccaggaatcaactgttcacgtcttggaaaaaggggcgacgacagtgcacttcagcacagctagccctcacgccagctttgtggtctcattatgcggaaagaagacgacctgcaccgcaggatgcaaaccacctgtcggccacatagtcagcacaccccacaagaaggaccaagagttccagaccgccgtctccaggacatcatggagctggctcttcgcgctgttcggaggagcttcgtccctagtaatcatgggactgttaatcttctcctgcagcatgatgctcactaacaccagaagatga

SEQ ID NO: 58 - DNA, 10325 bp, synthetic construct
  source 1..10325: mol_type = other DNA
aaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 59 - DNA, 10325 bp, synthetic construct
  source 1..10325: mol_type = other DNA
aaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 60 - RNA, 1103 nt, synthetic construct
  source 1..1103: mol_type = other RNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgt

SEQ ID NO: 61 - RNA, 1158 nt, synthetic construct
  source 1..1158: mol_type = other RNA
tggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcatta

SEQ ID NO: 62 - RNA, 1148 nt, synthetic construct
  source 1..1148: mol_type = other RNA
taccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaa

SEQ ID NO: 63 - RNA, 1169 nt, synthetic construct
  source 1..1169: mol_type = other RNA
gaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatg

SEQ ID NO: 64 - RNA, 1149 nt, synthetic construct
  source 1..1149: mol_type = other RNA
attgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagagga

SEQ ID NO: 65 - RNA, 1198 nt, synthetic construct
  source 1..1198: mol_type = other RNA
agaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggtta

SEQ ID NO: 66 - RNA, 1099 nt, synthetic construct
  source 1..1099: mol_type = other RNA
gctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcct

SEQ ID NO: 67 - DNA, 593 bp, synthetic construct
  source 1..593: mol_type = other DNA
cacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 68 - DNA, 988 bp, Streptomyces alboniger; identical to SEQ ID NO: 12
  source 1..988: mol_type = other DNA

SEQ ID NO: 69 - RNA, 578 nt, Homo sapiens
  source 1..578: mol_type = other RNA
gctgactgtgacttagttgcattacaccctttcttgacaaaacctaacttgcacagaaaacacgatgagattggcatggctttatttgtttttgtttttgtttgtttgtttgttttggcttgactcaggatttaaaaactggaacggtgaaggtgacagcagttggttggagcgagcatcccccaaagttctgcaatgtggccgagaactttgattgtacattgttctttttttaatggtcattccaaatatcgtgagatgcattgttacaggaagtcccttgcctcctaaaagccaccccacttctctctaaggagaatggcccagtcctctcccgagtccacacacgggaggtaatagcattgcttttgggtaaattatataatgcaaaaatcttttaatctttgccttaatacttttttatttttttaattttgaatgatcagcctttgtgactccccttttttgtcacccaacttgagatgtatgaaggcttttcatctccctcggagtgggtggaggcagccagggcttaactgtacactaacttgagaccagttgaataaaagtgcatacca

SEQ ID NO: 70 - RNA, 414 nt, Homo sapiens
  source 1..414: mol_type = other RNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaacca

SEQ ID NO: 71 - RNA, 90 nt, Homo sapiens
  source 1..90: mol_type = other RNA
accgccgagaccgcgtccgccccgcgagcacagagcctcgcctttgccgatccgccgcccgtccacacccgccgccagctcaccatggat

SEQ ID NO: 72 - RNA, 41 nt, Homo sapiens
  source 1..41: mol_type = other RNA
ctagcttttctcttctgtcaaccccacacgcctttggcaca

SEQ ID NO: 73 - RNA, 9305 nt, synthetic construct
  source 1..9305: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactggacctggcgaatactgttcttggttgccgccgctacagggactcacgcaatatgggagctgaagaaggacgtgtatgtcgtggagctggactggtacccagatgctcctggcgaaatggtggttttaacatgtgatacccccgaggaggacggcatcacatggactctggaccagagttctgaggtgctggggtccggcaagactctgacaatccaggttaaggagttcggcgacgcaggacagtacacttgtcacaagggaggtgaggtgctttctcacagcctgttgctgctccataagaaggaagacggtatttggtcaaccgacatcctcaaggaccagaaggagcccaaaaacaagacctttctgagatgtgaggccaagaattacagcggtagattcacttgttggtggctcaccaccatatccacagacttgaccttcagtgtcaaaagttcacgagggagctcagatcctcaaggcgttacctgtggcgcagcgacgctgtccgcagaaagagtcaggggagacaacaaggaatacgagtactctgtcgagtgccaggaggattccgcctgtccggccgcagaggagtctttacctattgaggtgatggtcgatgccgtgcacaagcttaagtacgagaattacacatcaagttttttcatccgcgacatcattaaacctgatccaccaaagaacctgcagctcaagcctctgaagaatagcaggcaggtcgaggtaagctgggagtatcctgatacctggtccaccccccacagttatttcagcctcaccttctgcgtccaagtccagggaaagagcaagagagagaagaaggatagggtgttcacagataagacttcagctactgtgatctgcagaaagaatgcgtctatctctgtgcgagcacaagacaggtactacagttctagctggagcgagtgggcatcagtcccctgcagtggtggcggaagcggagggggcagcggaggtgggagcggagggagcaggaacctcccagttgctacacctgacccgggaatgtttccatgcctccaccattcccagaatctcctccgagccgtgtccaatatgctgcaaaaggctcggcagaccttggagttttacccttgcacctcagaagaaatcgatcatgaggatatcacaaaggataagacgagcactgttgaggcatgcctgcccctggagctaactaagaatgagtcttgcctgaacagcagggagacttccttcattaccaacggtagctgtcttgccagcaggaagacatcttttatgatggccctgtgtctatctagcatatatgaagacctgaagatgtaccaggtggaattcaaaaccatgaatgctaagcttctcatggatcccaagaggcaaatcttcctggaccagaatatgcttgctgtcatagatgaactgatgcaggcgttgaattttaacagcgagacggtgcctcaaaaaagctcactggaagaacctgatttttataaaacgaagatcaagctgtgtattttactacacgcctttagaatccgcgctgttaccatcgacagagtaatgtcctacctaaatgcttcatagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 74 - RNA, 8096 nt, synthetic construct
  source 1..8096: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgtatcgaatgcaattgctgtcctgcattgcgctctctctggcgcttgtcaccaattcaaactgggttaacgtaatatctgatttgaaaaaaatcgaagacctcatacagagtatgcacatagatgctacgctgtacacagaaagcgacgtacacccgagctgtaaggtcactgccatgaagtgctttcttctcgaactccaagttatcagtctcgaatccggggatgcgtcaatccatgacactgttgagaatctcattattctcgcgaacaactcactcagctcaaatgggaacgtcactgaatccggctgcaaggaatgcgaggaattggaagagaagaatattaaagagttcctgcagagtttcgttcatattgttcaaatgtttatcaacacaagttagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 75 - RNA, 8096 nt, synthetic construct
  source 1..8096: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgtattcaatgcagcttgccagttgtgtcacactcactcttgtcctgctggttaattccaattggatcgatgtccggtatgatttggagaagatcgaatctctcatccaaagcatacacattgacaccactttgtatactgatagcgattttcacccatcttgtaaggtgaccgccatgaattgctttctgttggaacttcaagttatacttcacgagtattcaaacatgacactgaatgaaactgtccgaaacgtactctaccttgctaactccaccctttcatctaataagaacgtagctgagagcggatgtaaggagtgtgaggagctcgaagagaagacatttactgaatttcttcagagttttatccgcatagtgcaaatgtttattaacacctcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 76 - RNA, 8351 nt, synthetic construct
  source 1..8351: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgtatcgaatgcaattgctgtcctgcattgcgctctctctggcgcttgtcaccaattcaaactgggttaacgtaatatctgatttgaaaaaaatcgaagacctcatacagagtatgcacatagatgctacgctgtacacagaaagcgacgtacacccgagctgtaaggtcactgccatgaagtgctttcttctcgaactccaagttatcagtctcgaatccggggatgcgtcaatccatgacactgttgagaatctcattattctcgcgaacaactcactcagctcaaatgggaacgtcactgaatccggctgcaaggaatgcgaggaattggaagagaagaatattaaagagttcctgcagagtttcgttcatattgttcaaatgtttatcaacacaagtggaggggggtcagggggtggctcaggcggcggcagtgggggcagcataacttgtccacctccgatgagtgtggaacatgctgacatttgggtaaagagttactcactctattcacgagagcgatacatctgtaatagtgggtttaaaagaaaagccggtacatcctctcttactgaatgcgtactcaataaggcgaccaacgtcgcgcactggaccactccttccttgaaatgcatccgcgacccggctctcgtttagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 77 - RNA, 8351 nt, synthetic construct
  source 1..8351: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgtaccgcatgcaacttttgtcttgcattgcacttagcttggcactcgtcactaattccattacatgccctccgcctatgtctgtggagcacgcagacatctgggtaaagtcttactccctctactcaagagaacgatatatctgtaattcagggtttaagcgaaaagcaggaacctcttcactgacagaatgcgtgcttaataaggcgacgaacgttgctcattggactacacctagtttgaagtgtattcgggatccggcactcgtaggaggggggtcagggggtggctcaggcggcggcagtgggggcagcaattgggtaaacgtaattagcgatctgaagaagatcgaggacttgatacaaagtatgcatatcgatgctactctttataccgagtctgatgtacacccctcttgtaaagtaacagcgatgaagtgctttttgttggagttgcaagtgataagcctggaaagtggggatgcgtccatccacgataccgtcgagaacttgataatactggccaacaacagtctctctagtaacggtaacgtaaccgagagcgggtgtaaagaatgcgaagaattggaagagaaaaatatcaaagaattccttcagtcatttgtgcatatagtgcagatgtttatcaacacatcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 78 - RNA, 8354 nt, synthetic construct
  source 1..8354: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgtattcaatgcagcttgccagttgtgtcacactcactcttgtcctgctggttaattccaattggatcgatgtccggtatgatttggagaagatcgaatctctcatccaaagcatacacattgacaccactttgtatactgatagcgattttcacccatcttgtaaggtgaccgccatgaattgctttctgttggaacttcaagttatacttcacgagtattcaaacatgacactgaatgaaactgtccgaaacgtactctaccttgctaactccaccctttcatctaataagaacgtagctgagagcggatgtaaggagtgtgaggagctcgaagagaagacatttactgaatttcttcagagttttatccgcatagtgcaaatgtttattaacacctccggaggggggtcagggggtggctcaggcggcggcagtgggggcagcggaactacatgtccacctcccgtcagtatcgaacatgcagatatcagagttaaaaattattcagtgaacagcagggagcgctacgtgtgtaatagtggttttaaaaggaaagccggcacttctacattgattgaatgcgttatcaataagaatactaacgtagctcactggaccacaccctctctgaagtgtatccgagacccttcacttgcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 79 - RNA, 8231 nt, synthetic construct
  source 1..8231: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgacctcccggcttgtgagggtactggctgctgctatgctggtggctgctgctgtgagtgtggcaaactttgggagactccactgtaccactgccgttatacggaatataaacgatcaggtcctctttgtagacaagagacagccagttttcgaggacatgactgatatagatcagagtgcctctgagcctcaaactcggctcataatatacatgtataaggatagcgaggttcggggacttgccgtgactttgtccgtgaaagactcaaaaatgagcaccctgtcatgcaaaaataagataatttctttcgaggaaatggaccctcccgagaacattgatgatatccagtccgacttgatatttttccagaaacgggttcccggtcataacaaaatggagtttgagtcttcattgtacgagggtcattttttggcatgccagaaggaggatgatgccttcaaactgattttgaagaaaaaagacgagaacggagacaagtctgtcatgtttacacttaccaaccttcatcaatcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 80 - RNA, 8231 nt, synthetic construct
  source 1..8231: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgacctcccggcttgtgagggtactggctgctgctatgctggtggctgctgctgtgagtgtggcacacttcggacgactccattgtactacagccgttatacgaaatatcaatgatcaggtgttgtttgtagacaaaaggcagcccgtgtttgaagacatgactgatattgatcagagtgcctctgagccccagactagactgattatatatgcttatggcgactctagagctagagggaaagcagtaactctgtcagtgaaagacagcaagatgtctaccctgtcttgcaagaataaaataatctcatttgaggagatggacccccctgaaaacatagatgacatccaaagtgacctgatattctttcagaagcgggtacccggtcataacaaaatggagttcgagtcaagcctctatgagggtcactttctcgcctgccaaaaggaagatgacgccttcaaattgatcttgaaaaaaaaagacgagaatggtgataagtctgtcatgttcacactcactaatttgcatcagtcatagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 81 - RNA, 8948 nt, synthetic construct
  source 1..8948: mol_type = other RNA
gcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatgaatccatccgcagctgtaattttctgccttatcctcctggggctgagtggaacccaaggaatccccctcgctcgaacagtacgctgcaactgtatccacatagacgatggccccgttcgaatgagggctatcggaaagctcgaaataatacccgcctctctctcatgcccccgcgtagaaataatagcaactatgaagaagaacgacgagcaacgatgcttgaacccagaaagcaagactatcaagaacttgatgaaagcttttagccaaaagcggagtaaacgagcccccgggggaggcggctccggcggaggagggagtggtggcggtggatccggaggcggtgggagtacccttgtgattaggaatgcacgatgcagctgtataagcacctcccggggcactattcactataagagccttaaagatctcaagcagtttgcaccatctccaaattgtaacaaaactgagattattgctacactgaagaacggggaccaaacctgcctggacccagatagtgccaatgttaaaaagcttatgaaggaatgggagaaaaaaatcagtcaaaagaaaaagcaaaaacgcggtaaaaaacaccagaaaaacatgaagaatcggaaaccaaaaaccccacagtctaggagaaggagtcgaaaaacaacagggggaggcggaagtggcggcgggggcagcggtggcgggggatcaggcggcgggggcagtttccttatgttcaaacaagggcggtgcctgtgtatagggccaggtatgaaagcagtgaagatggctgaaatcgaaaaagcaagtgtgatctaccctagtaatggctgtgataaagttgaagttattgtgactatgaaagctcacaagagacagcggtgtttggatcctagatccaaacaagcccgactcatcatgcaagccatagaaaagaagaacttcctgcgaaggcagaatatgggtggaggtggaagcggtggcgggggtagcggcgggggaggtagtggtgggggcggtagcgtaactagtgctggaccagaggagtccgatggggatctctcttgtgtctgcgttaagaccatatcatccgggattcaccttaagcacattaccagcctcgaagtgatcaaagctggtaggcattgtgccgtgccccagctcatagccacattgaagaatgggaggaaaatttgccttgatagacaggcaccattgtataagaaagtcattaaaaaaattcttgaatcatagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaa

SEQ ID NO: 82 - RNA, 13 nt, unidentified
  source 1..13: mol_type = other RNA
atttaggtgacac

SEQ ID NO: 83 - RNA, 13 nt, unidentified
  source 1..13: mol_type = other RNA
aattagggcacac

SEQ ID NO: 84 - DNA, 10325 bp, synthetic construct
  source 1..10325: mol_type = other DNA
aaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 85 - DNA, 6101 bp, synthetic construct
  source 1..6101: mol_type = other DNA
agccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggacatttgcttctgacacaactgtgttcactagcaacctcaaacagacaccatgaattacatccctacgcaaacgttttacgggcgcagatggcgacctcgacccgccgctcgcccctggcctttgcaagctacgcccgtagcccccgtggtcccagactttcaggcacagcaaatgcagcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaaggggagggtagtcgctatcgtcctgggcggggccaacgagggctcacgcacagcactgtcggtggtcacctggaacaaagatatggtgactagagtgacccccgaggggtccgaagagtggtccgccccgctgattactgccatgtgtgtccttgccaatgctaccttcccgtgcttccagcccccgtgtgtaccttgctgctatgaaaacaacgcagaggccacactacggatgctcgaggataacgtggataggccagggtactacgacctccttcaggcagccttgacgtgccgaaacggaacaagacaccggcgcagcgtgtcgcaacacttcaacgtgtataaggctacacgcccttacatcgcgtactgcgccgactgcggagcagggcactcgtgtcatagccccgtagcaattgaagcggtcaggtccgaagctaccgacgggatgctgaagattcagttctcggcacaaattggcatagataagagtgacaatcatgactacacgaagataaggtacgcagacgggcacgccattgagaatgccgtccggtcatctttgaaggtagccacctccggagactgtttcgtccatggcacaatgggacatttcatactggcaaagtgcccaccgggtgaattcctgcaggtctcgatccaggacaccagaaacgcggtccgtgcctgcagaatacaatatcatcatgaccctcaaccggtgggtagagaaaaatttacaattagaccacactatggaaaagagatcccttgcaccacttatcaacagaccacagcgaagaccgtggaggaaatcgacatgcatatgccgccagatacgccggacaggacgttgctatcacagcaatctggcaatgtaaagatcacagtcggaggaaagaaggtgaaatacaactgcacctgtggaaccggaaacgttggcactactaattcggacatgacgatcaacacgtgtctaatagagcagtgccacgtctcagtgacggaccataagaaatggcagttcaactcacctttcgtcccgagagccgacgaaccggctagaaaaggcaaagtccatatcccattcccgttggacaacatcacatgcagagttccaatggcgcgcgaaccaaccgtcatccacggcaaaagagaagtgacactgcaccttcacccagatcatcccacgctcttttcctaccgcacactgggtgaggacccgcagtatcacgaggaatgggtgacagcggcggtggaacggaccatacccgtaccagtggacgggatggagtaccactggggaaacaacgacccagtgaggctttggtctcaactcaccactgaagggaaaccgcacggctggccgcatcagatcgtacagtactactatgggctttacccggccgctacagtatccgcggtcgtcgggatgagcttactggcgttgatatcgatcttcgcgtcgtgctacatgctggttgcggcccgcagtaagtgcttgaccccttatgctttaacaccaggagctgcagttccgtggacgctggggatactctgctgcgccccgcgggcgcacgcagctagtgtggcagagactatggcctacttgtgggaccaaaaccaagcgttgttctggttggagtttgcggcccctgttgcctgcatcctcatcatcacgtattgcctcagaaacgtgctgtgttgctgtaagagcctttcttttttagtgctactgagcctcggggcaaccgccagagcttacgaacattcgacagtaatgccgaacgtggtggggttcccgtataaggctcacattgaaaggccaggatatagccccctcactttgcagatgcaggttgttgaaaccagcctcgaaccaacccttaatttggaatacataacctgtgagtacaagacggtcgtcccgtcgccgtacgtgaagtgctgcggcgcctcagagtgctccactaaagagaagcctgactaccaatgcaaggtttacacaggcgtgtacccgttcatgtggggaggggcatattgcttctgcgactcagaaaacacgcaactcagcgaggcgtacgtcgatcgatcggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccgacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 86 - DNA, 12006 bp, synthetic construct
  source 1..12006: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagttcctcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagtggaggggggtcagggggtggctcaggcggcggcagtgggggcagcagggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 87 - DNA, 11997 bp, synthetic construct
  source 1..11997: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatatgggagctgaagaaggacgtgtatgtcgtggagctggactggtacccagatgctcctggcgaaatggtggttttaacatgtgatacccccgaggaggacggcatcacatggactctggaccagagttctgaggtgctggggtccggcaagactctgacaatccaggttaaggagttcggcgacgcaggacagtacacttgtcacaagggaggtgaggtgctttctcacagcctgttgctgctccataagaaggaagacggtatttggtcaaccgacatcctcaaggaccagaaggagcccaaaaacaagacctttctgagatgtgaggccaagaattacagcggtagattcacttgttggtggctcaccaccatatccacagacttgaccttcagtgtcaaaagttcacgagggagctcagatcctcaaggcgttacctgtggcgcagcgacgctgtccgcagaaagagtcaggggagacaacaaggaatacgagtactctgtcgagtgccaggaggattccgcctgtccggccgcagaggagtctttacctattgaggtgatggtcgatgccgtgcacaagcttaagtacgagaattacacatcaagttttttcatccgcgacatcattaaacctgatccaccaaagaacctgcagctcaagcctctgaagaatagcaggcaggtcgaggtaagctgggagtatcctgatacctggtccaccccccacagttatttcagcctcaccttctgcgtccaagtccagggaaagagcaagagagagaagaaggatagggtgttcacagataagacttcagctactgtgatctgcagaaagaatgcgtctatctctgtgcgagcacaagacaggtactacagttctagctggagcgagtgggcatcagtcccctgcagtggtggcggaagcggagggggcagcggaggtgggagcggagggagcaggaacctcccagttgctacacctgacccgggaatgtttccatgcctccaccattcccagaatctcctccgagccgtgtccaatatgctgcaaaaggctcggcagaccttggagttttacccttgcacctcagaagaaatcgatcatgaggatatcacaaaggataagacgagcactgttgaggcatgcctgcccctggagctaactaagaatgagtcttgcctgaacagcagggagacttccttcattaccaacggtagctgtcttgccagcaggaagacatcttttatgatggccctgtgtctatctagcatatatgaagacctgaagatgtaccaggtggaattcaaaaccatgaatgctaagcttctcatggatcccaagaggcaaatcttcctggaccagaatatgcttgctgtcatagatgaactgatgcaggcgttgaattttaacagcgagacggtgcctcaaaaaagctcactggaagaacctgatttttataaaacgaagatcaagctgtgtattttactacacgcctttagaatccgcgctgttaccatcgacagagtaatgtcctacctaaatgcttcatagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 88 - RNA, 6079 nt, synthetic construct
  source 1..6079: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggacatttgcttctgacacaactgtgttcactagcaacctcaaacagacaccatgaatagaggaatatttaacatgctgagccgccgccccttccccgcccccacagctatgtggaggcctaggcgaaggaggcaggcggcaccactgcccgcccgtaatgggttggcatcccaaattcaacagctaaccacagctgtcagtgccctagtcattggtcaagccacccgacctcaacaaccaaggccacgcccgctaccacgccctaagaagcagtccccgaagcaaccaccgaatgctaaaaagacaaaaccgcaggagaacaaggagaaacaacccgcaaaaccgaagccgggtaagagacagcgtatggcactaaaattgatgctgacaggactctttgacgtgaacaacgaagccggtgacgtaataggacacgccctcgctatggaaggcaaagtgatgaagccacttcacgtgaaaggggtcatagaccacccggttttggcaaagctgaaatttaccaagtcatcagcgtacgacatggagttcgcccagcttccagccagtatgaaaagcgaagcatttgcatacaccagcgagcaccctgaaggcttctacaactggcatcatggtgccgtgcagtacagcggcggtcgctttacagtcccgcgcggagttgggggaaaaggcgacagtggaagaccgatcatggataatacaggtaaggtggtcgcgatagtgctgggcggcgctgacgaaggcgcccgcactgctctctccgtcgtcacttggaacagcaaaggaaagaccatcaaaacgactccggaagggactgaggaatggtctgcagcaccattagtcacggctatgtgcttctttgagaatgtatcgttcccgtgcagtcgtccgcctacgtgctattcccgaaaaccatctcgtgcccttgacatcctggaggagaacgtgaacaacgacgcctacgataccttattagcggcagtgttgaagtgcaggacgtccggtcggaacaaacgcagcatcaccgacgatttcaccctgaccagcccgtacctgggcacttgctcgtattgccatcatactgaaccctgcttcagcccaattaagatcgagcaagtttgggacgaggcggacgacggatcaatacgaatacagacatccgcccagttcgggtacgaccaaagtggggcagcagacgtcaacaagtacaggtacatgtcaattgaccaggaccatgccgtgaaagaaggctctatggatcatatcaagatcagcacatccgggccctgtaggcggcttcaatcacaaaggtacttcttgcttgcgaaatgcccaccgggagatagcgttacggttagcataaccggcggcactatggccacctcctgtacattagcacgtaaaataaggcaaaaattcgtaggacgtgagaagtatgaccttcccccagcacacggaaagaacatcccctgcaccgtgtatgatcgtcttaaggagacgtcagccggatacatcaccatgcacaggcctggacctcatgcctatacctcatacctagaggctacctcaggaaaaatttacgccaaaccaccatcaggaaagaacatcacgtacgagtgtaagtgtggcgattataaaactgcgaccgtttccgtcagaacagagatcgcaggatgtactgccataaaacagtgcgtggcgtacaagagcgaccagaccaagtgggtgttcaactcgcccgatctaatcaggcacgctgaccacacagcccaaggtaaattccactttcctttcaagcctaccttcagtacatgcctggtaccactggcacacgagccgactgtaattcatgggttcaaacacataagcctccatttagacacagaccacccaaccttgctcaccattagaagactgggcgagaaacccgaaccgacgtcggagtggatctcaggaaaaacagttaggaattttactgtcgacagagacgggctagagtatatatggggcaatcacgaaccggtgagagtatatgcacaagagtccgcccccggtgaccctcacggatggccacacaaaataattcaacactactatcaacgccacccagtatacactagcttggctgtagcagcttccgctgtggcggtgatagtcggcctggccaccgccgtgatgtgtatctgcagagcgcgacgggagtgcctaaccccgtacgcgttagccccgaatgcagtcatcccgacgtccttggccttattgtgctgcgttcggtccgccaacgccgaatctttcaccgacacgatgggatacctatggtcgaacagtcaaacatttttctgggtgcagctgtgcatccccttggcagtagtaatagtactcgtacgctgctgttcgtgctgcctcccttttttagtggttgcaggcgcctacctggcgaaggtagacgccttcgaacatgcgaccactgtcccaaatgtgccgaagattccgtataaagctttggtcgagagagccggatacgccccgcttaacttggaaattactgttatgtcttcagaggtcctcccttcgattcaccaagaatacatcacctgcaaatactccactatcgtgccttccccgaaagtgaaatgctgcggttcccttgagtgccaacccgcttcgcatgccgactacaactgcaaagtatttggcggcgtttaccctttcatgtggggtggagcgcaatgtttttgcgacagcgagaacagccagatgagtgaggcgtatgtcgagctatcggccgattgtgcccttgatcacgcacaggcggtgaaagtgcacacagccgcaatgaaggtcggactacgcattgtgtatgggaacaccactagcctcctggatgcgtacgtaaacggagtcacgccaggcacatctaaagacctgaaagtcattgccggacccatctcgtccgcgttttccccttttgactctaaagtcgtgatacaccgagggctcgtgtacaattacgattttccagagtacggggcaatgaaaccgggtgcctttggggacattcaagctagctctttgacgggcaacgacctgattgccagcaccgacatacgtttgcttaaacccgcagcaaagaatgtgcacgttccctatacccaagcagcatcaggatttgaaatttggaagaacaactccggaagaccactgcaagagacggccccatttggctgtaagattgccgtcaaccctctgaaggccgtggattgttcctatgggaacatcccgatatcgattgatatcccccacgccgcctttattagaccatccgatgcgccatttatctccgaagtgaagtgcgaagtcagcgactgcacatactctgctgacttcggaggcatggccaccctacagtatgtgtctgaccgagagggccagtgcccagtgcattcccattcaagtaccgccacactccaggaatcaactgttcacgtcttggaaaaaggggcgacgacagtgcacttcagcacagctagccctcacgccagctttgtggtctcattatgcggaaagaagacgacctgcaccgcaggatgcaaaccacctgtcggccacatagtcagcacaccccacaagaaggaccaagagttccagaccgccgtctccaggacatcatggagctggctcttcgcgctgttcggaggagcttcgtccctagtaatcatgggactgttaatcttctcctgcagcatgatgctcactaacaccagaagatga

SEQ ID NO: 89 - RNA, 6047 nt, synthetic construct
  source 1..6047: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggacatttgcttctgacacaactgtgttcactagcaacctcaaacagacaccatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattga

SEQ ID NO: 90 - DNA, 10145 bp, synthetic construct
  source 1..10145: mol_type = other DNA
aaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatgacctcccggcttgtgagggtactggctgctgctatgctggtggctgctgctgtgagtgtggcaaactttgggagactccactgtaccactgccgttatacggaatataaacgatcaggtcctctttgtagacaagagacagccagttttcgaggacatgactgatatagatcagagtgcctctgagcctcaaactcggctcataatatacatgtataaggatagcgaggttcggggacttgccgtgactttgtccgtgaaagactcaaaaatgagcaccctgtcatgcaaaaataagataatttctttcgaggaaatggaccctcccgagaacattgatgatatccagtccgacttgatatttttccagaaacgggttcccggtcataacaaaatggagtttgagtcttcattgtacgagggtcattttttggcatgccagaaggaggatgatgccttcaaactgattttgaagaaaaaagacgagaacggagacaagtctgtcatgtttacacttaccaaccttcatcaatcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 91 - DNA, 10145 bp, synthetic construct
  source 1..10145: mol_type = other DNA
aaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatgacctcccggcttgtgagggtactggctgctgctatgctggtggctgctgctgtgagtgtggcacacttcggacgactccattgtactacagccgttatacgaaatatcaatgatcaggtgttgtttgtagacaaaaggcagcccgtgtttgaagacatgactgatattgatcagagtgcctctgagccccagactagactgattatatatgcttatggcgactctagagctagagggaaagcagtaactctgtcagtgaaagacagcaagatgtctaccctgtcttgcaagaataaaataatctcatttgaggagatggacccccctgaaaacatagatgacatccaaagtgacctgatattctttcagaagcgggtacccggtcataacaaaatggagttcgagtcaagcctctatgagggtcactttctcgcctgccaaaaggaagatgacgccttcaaattgatcttgaaaaaaaaagacgagaatggtgataagtctgtcatgttcacactcactaatttgcatcagtcatagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 92 - DNA, 10923 bp, synthetic construct
  source 1..10923: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggacatgagagtcccagctcaactgcttggtcttcttctgctgtggttgcgcggtgccagatgttactttggaaagcttgagtccaaactttctgtcataagaaacttgaacgaccaggtgttgtttatagaccagggtaatcgacctctcttcgaggatatgacagatagcgactgcagagataacgccccccgcaccatattcataatatccatgtataaggactcacaaccaagaggcatggccgtaacaataagcgtaaaatgtgagaaaatctctaccctgagctgtgagaacaagatcataagcttcaaggaaatgaacccaccagacaacatcaaggatactaaaagtgacattatcttcttccaacggagcgtcccaggccacgacaacaagatgcagttcgagtccagcagttacgaaggttacttcctggcgtgcgagaaagagcgcgacctttttaagctcattctgaagaaggaagatgagttgggtgacaggagcattatgttcacggtccagaatgaggactagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 93 - DNA, 10923 bp, synthetic construct
  source 1..10923: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggacatgagagtcccagctcaactgcttggtcttcttctgctgtggttgcgcggtgccagatgtcacttcgggaagttggagtccaagttgagtgttattcgcaacctcaatgatcaggtgttgttcatagaccagggtaaccgcccactttttgaagatatgaccgatagcgattgccgcgacaacgcccccaggaccatcttcataataagtgcgtacggagactcacgggcccgggggaaggctgttacaatatccgtaaagtgcgagaagatctccacgctttcttgcgaaaataagattatcagttttaaagaaatgaaccctcctgacaacataaaagatacgaaatcagacatcatcttcttccagcgctcagttcctggacatgataataagatgcagttcgagtcatctagctatgaagggtattttctggcttgtgagaaagaacgggatttgtttaaacttatactgaagaaggaggatgaacttggtgatcggtctattatgtttaccgtacaaaatgaggactagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 94 - DNA, 13166 bp, synthetic construct
  source 1..13166: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagttcctcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagtggaggggggtcagggggtggctcaggcggcggcagtgggggcagcagggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcctaggcgatcgcgctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataacgcgttgccaccatgacctcccggcttgtgagggtactggctgctgctatgctggtggctgctgctgtgagtgtggcacacttcggacgactccattgtactacagccgttatacgaaatatcaatgatcaggtgttgtttgtagacaaaaggcagcccgtgtttgaagacatgactgatattgatcagagtgcctctgagccccagactagactgattatatatgcttatggcgactctagagctagagggaaagcagtaactctgtcagtgaaagacagcaagatgtctaccctgtcttgcaagaataaaataatctcatttgaggagatggacccccctgaaaacatagatgacatccaaagtgacctgatattctttcagaagcgggtacccggtcataacaaaatggagttcgagtcaagcctctatgagggtcactttctcgcctgccaaaaggaagatgacgccttcaaattgatcttgaaaaaaaaagacgagaatggtgataagtctgtcatgttcacactcactaatttgcatcagtcatagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 95 - DNA, 13166 bp, synthetic construct
  source 1..13166: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatgtgggagcttgaaaaagacgtctatgtagtagaagtggactggacacctgatgctcctggcgagacagttaacctcacatgcgatacccctgaggaagatgatatcacctggacttctgaccagagacacggggtgattgggagcggcaaaaccctgacgatcactgtgaaggagtttctggacgccggccagtatacctgtcacaaggggggggagaccctgagtcatagccacctgttgctgcacaagaaggagaatggcatctggtctacagagatcctgaagaactttaagaacaagaccttcctgaagtgtgaagcaccaaactacagtggtcgctttacctgcagctggctggtccaaagaaacatggacctgaaatttaatataaagagtagttcctcgagtcctgattccagggccgtgacgtgcggcatggcaagcctttcagccgaaaaagtcacgctggatcagcgagactatgagaagtacagcgttagctgtcaggaggacgtaacttgcccgactgccgaggagactctgcccatagagctcgctctggaggccaggcagcagaacaaatatgagaattacagcactagtttctttattagagacatcatcaaacccgacccacccaagaatctgcagatgaagccgctgaagaatagtcaggtcgaggtttcctgggaatatccagattcatggtccactccgcattcttatttttccttaaaattctttgttaggattcagcggaaaaaagaaaagatgaaagagacggaggaagggtgcaaccagaagggggccttcctggtggaaaagacaagcactgaggtccaatgtaagggtgggaacgtttgcgtgcaggctcaggatcgctactacaacagcagttgctctaagtgggcctgcgtaccttgtcgcgtcaggagtggaggggggtcagggggtggctcaggcggcggcagtgggggcagcagggtgatcccagtgtctgggccggcccgttgcttgtctcaatccagaaacctcctcaagaccactgacgatatggtaaagactgcccgagagaagctaaaacactactcttgtacagctgaagatatagaccatgaggatataacacgggaccagacctctactctgaaaacctgtctgcctcttgagctgcacaagaacgagtcctgtctggctacccgcgaaacctcaagcacaaccagaggtagttgcctgcccccacaaaagacatcgcttatgatgaccttgtgtctgggatctatttatgaggacctgaagatgtaccaaactgagttccaggcaataaatgctgctctccagaatcacaatcatcaacaaatcatccttgataaggggatgctggtcgcaatcgacgagctcatgcaatcgctgaaccacaatggggaaaccctcaggcagaaaccaccggtgggagaggccgacccctaccgtgttaaaatgaagttgtgtattcttttgcatgcattctctacaagagtcgttaccatcaatcgcgtcatggggtacctgtcatcagcctaggcgatcgcgctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataacgcgttgccaccatgacctcccggcttgtgagggtactggctgctgctatgctggtggctgctgctgtgagtgtggcaaactttgggagactccactgtaccactgccgttatacggaatataaacgatcaggtcctctttgtagacaagagacagccagttttcgaggacatgactgatatagatcagagtgcctctgagcctcaaactcggctcataatatacatgtataaggatagcgaggttcggggacttgccgtgactttgtccgtgaaagactcaaaaatgagcaccctgtcatgcaaaaataagataatttctttcgaggaaatggaccctcccgagaacattgatgatatccagtccgacttgatatttttccagaaacgggttcccggtcataacaaaatggagtttgagtcttcattgtacgagggtcattttttggcatgccagaaggaggatgatgccttcaaactgattttgaagaaaaaagacgagaacggagacaagtctgtcatgtttacacttaccaaccttcatcaatcctagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 96 - DNA, 13157 bp, synthetic construct
  source 1..13157: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatatgggagctgaagaaggacgtgtatgtcgtggagctggactggtacccagatgctcctggcgaaatggtggttttaacatgtgatacccccgaggaggacggcatcacatggactctggaccagagttctgaggtgctggggtccggcaagactctgacaatccaggttaaggagttcggcgacgcaggacagtacacttgtcacaagggaggtgaggtgctttctcacagcctgttgctgctccataagaaggaagacggtatttggtcaaccgacatcctcaaggaccagaaggagcccaaaaacaagacctttctgagatgtgaggccaagaattacagcggtagattcacttgttggtggctcaccaccatatccacagacttgaccttcagtgtcaaaagttcacgagggagctcagatcctcaaggcgttacctgtggcgcagcgacgctgtccgcagaaagagtcaggggagacaacaaggaatacgagtactctgtcgagtgccaggaggattccgcctgtccggccgcagaggagtctttacctattgaggtgatggtcgatgccgtgcacaagcttaagtacgagaattacacatcaagttttttcatccgcgacatcattaaacctgatccaccaaagaacctgcagctcaagcctctgaagaatagcaggcaggtcgaggtaagctgggagtatcctgatacctggtccaccccccacagttatttcagcctcaccttctgcgtccaagtccagggaaagagcaagagagagaagaaggatagggtgttcacagataagacttcagctactgtgatctgcagaaagaatgcgtctatctctgtgcgagcacaagacaggtactacagttctagctggagcgagtgggcatcagtcccctgcagtggtggcggaagcggagggggcagcggaggtgggagcggagggagcaggaacctcccagttgctacacctgacccgggaatgtttccatgcctccaccattcccagaatctcctccgagccgtgtccaatatgctgcaaaaggctcggcagaccttggagttttacccttgcacctcagaagaaatcgatcatgaggatatcacaaaggataagacgagcactgttgaggcatgcctgcccctggagctaactaagaatgagtcttgcctgaacagcagggagacttccttcattaccaacggtagctgtcttgccagcaggaagacatcttttatgatggccctgtgtctatctagcatatatgaagacctgaagatgtaccaggtggaattcaaaaccatgaatgctaagcttctcatggatcccaagaggcaaatcttcctggaccagaatatgcttgctgtcatagatgaactgatgcaggcgttgaattttaacagcgagacggtgcctcaaaaaagctcactggaagaacctgatttttataaaacgaagatcaagctgtgtattttactacacgcctttagaatccgcgctgttaccatcgacagagtaatgtcctacctaaatgcttcataggcgatcgcgctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataacgcgttgccaccatggacatgagagtcccagctcaactgcttggtcttcttctgctgtggttgcgcggtgccagatgttactttggaaagcttgagtccaaactttctgtcataagaaacttgaacgaccaggtgttgtttatagaccagggtaatcgacctctcttcgaggatatgacagatagcgactgcagagataacgccccccgcaccatattcataatatccatgtataaggactcacaaccaagaggcatggccgtaacaataagcgtaaaatgtgagaaaatctctaccctgagctgtgagaacaagatcataagcttcaaggaaatgaacccaccagacaacatcaaggatactaaaagtgacattatcttcttccaacggagcgtcccaggccacgacaacaagatgcagttcgagtccagcagttacgaaggttacttcctggcgtgcgagaaagagcgcgacctttttaagctcattctgaagaaggaagatgagttgggtgacaggagcattatgttcacggtccagaatgaggactagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 97 - DNA, 13157 bp, synthetic construct
  source 1..13157: mol_type = other DNA
aaaaaaaaaacgcggaagagccgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcgatccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggactggacctggcgaatactgttcttggttgccgccgctacagggactcacgcaatatgggagctgaagaaggacgtgtatgtcgtggagctggactggtacccagatgctcctggcgaaatggtggttttaacatgtgatacccccgaggaggacggcatcacatggactctggaccagagttctgaggtgctggggtccggcaagactctgacaatccaggttaaggagttcggcgacgcaggacagtacacttgtcacaagggaggtgaggtgctttctcacagcctgttgctgctccataagaaggaagacggtatttggtcaaccgacatcctcaaggaccagaaggagcccaaaaacaagacctttctgagatgtgaggccaagaattacagcggtagattcacttgttggtggctcaccaccatatccacagacttgaccttcagtgtcaaaagttcacgagggagctcagatcctcaaggcgttacctgtggcgcagcgacgctgtccgcagaaagagtcaggggagacaacaaggaatacgagtactctgtcgagtgccaggaggattccgcctgtccggccgcagaggagtctttacctattgaggtgatggtcgatgccgtgcacaagcttaagtacgagaattacacatcaagttttttcatccgcgacatcattaaacctgatccaccaaagaacctgcagctcaagcctctgaagaatagcaggcaggtcgaggtaagctgggagtatcctgatacctggtccaccccccacagttatttcagcctcaccttctgcgtccaagtccagggaaagagcaagagagagaagaaggatagggtgttcacagataagacttcagctactgtgatctgcagaaagaatgcgtctatctctgtgcgagcacaagacaggtactacagttctagctggagcgagtgggcatcagtcccctgcagtggtggcggaagcggagggggcagcggaggtgggagcggagggagcaggaacctcccagttgctacacctgacccgggaatgtttccatgcctccaccattcccagaatctcctccgagccgtgtccaatatgctgcaaaaggctcggcagaccttggagttttacccttgcacctcagaagaaatcgatcatgaggatatcacaaaggataagacgagcactgttgaggcatgcctgcccctggagctaactaagaatgagtcttgcctgaacagcagggagacttccttcattaccaacggtagctgtcttgccagcaggaagacatcttttatgatggccctgtgtctatctagcatatatgaagacctgaagatgtaccaggtggaattcaaaaccatgaatgctaagcttctcatggatcccaagaggcaaatcttcctggaccagaatatgcttgctgtcatagatgaactgatgcaggcgttgaattttaacagcgagacggtgcctcaaaaaagctcactggaagaacctgatttttataaaacgaagatcaagctgtgtattttactacacgcctttagaatccgcgctgttaccatcgacagagtaatgtcctacctaaatgcttcataggcgatcgcgctagcaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataacgcgttgccaccatggacatgagagtcccagctcaactgcttggtcttcttctgctgtggttgcgcggtgccagatgtcacttcgggaagttggagtccaagttgagtgttattcgcaacctcaatgatcaggtgttgttcatagaccagggtaaccgcccactttttgaagatatgaccgatagcgattgccgcgacaacgcccccaggaccatcttcataataagtgcgtacggagactcacgggcccgggggaaggctgttacaatatccgtaaagtgcgagaagatctccacgctttcttgcgaaaataagattatcagttttaaagaaatgaaccctcctgacaacataaaagatacgaaatcagacatcatcttcttccagcgctcagttcctggacatgataataagatgcagttcgagtcatctagctatgaagggtattttctggcttgtgagaaagaacgggatttgtttaaacttatactgaagaaggaggatgaacttggtgatcggtctattatgtttaccgtacaaaatgaggactagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 98 - DNA, 11258 bp, synthetic construct
  source 1..11258: mol_type = other DNA
aaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagttcgaaggcgcgcctctagagccaccatggaggacgctaaaaatatcaagaagggtcccgctcctttctatccactcgaggacggtaccgctggtgagcaactgcacaaggctatgaaacggtatgctcttgtgccaggcaccatagctttcaccgacgcccacatagaggtggacattacttacgcagagtactttgagatgtctgtccggctggcagaggcaatgaaaagatatggcctcaacacaaaccataggatagtggtttgcagcgagaattctcttcagttttttatgcccgtcctcggggctcttttcatcggcgtggctgtcgcccccgccaacgacatatacaacgagcgagagctgctgaacagcatgggaatcagtcagcctacagtggtttttgtatcaaagaagggcttgcaaaagatccttaacgtacagaaaaagctccctattattcagaagatcattataatggattcaaagaccgactatcagggattccagtcaatgtatactttcgtcacttcccaccttcctcctgggtttaatgaatacgacttcgttcccgaaagtttcgatcgggacaaaactattgcactcatcatgaactcctctgggtccactgggctgcctaaaggtgtcgcacttccccacaggactgcatgtgtaaggttctctcacgcacgagatcccatattcggtaaccaaatcattcctgataccgctatcctctctgtagtgcctttccaccatggattcggcatgttcaccactctgggttacttgatatgtgggttccgcgtggtccttatgtacagatttgaagaggagctctttttgaggagcttgcaggactacaaaatccaaagtgctctcctcgtccccacattgttctcattcttcgccaaatctactctcatcgacaagtacgatttgagcaacttgcacgaaattgcttctgggggggctccactctccaaagaagtcggcgaagccgttgctaaaaggttccacttgccaggcattcgccaggggtatggccttactgagacaacctcagctattctcattacccctgagggtgacgataagcctggtgccgtcggtaaggtagtaccatttttcgaggcaaaggtggtcgacttggataccggaaaaaccctcggtgtgaatcagagaggcgaattgtgtgtcaggggtccaatgatcatgagcggttacgtcaataatcccgaggccactaacgccttgattgataaagatggttggcttcattcaggtgatatagcatactgggatgaggatgaacatttcttcattgtcgataggctcaaaagcctgatcaagtataagggttaccaagtggcccctgccgaactcgaatctatactgctgcaacatccaaacatttttgacgctggcgtggccggactgcccgatgatgatgctggtgaactccctgccgccgttgtcgtcctggaacatggtaagaccatgacagaaaaggagattgtagattacgtggcttcacaagtgactacagctaagaaattgcgcgggggtgtcgttttcgtggacgaggtacccaagggcctcactgggaagctcgacgccagaaaaatccgagaaatcctgattaaagcaaagaagggcggaaagattgcagtgtagcatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaa

SEQ ID NO: 99 - DNA, 3056 bp, synthetic construct
  source 1..3056: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggacatttgcttctgacacaactgtgttcactagcaacctcaaacagacaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtag

SEQ ID NO: 100 - DNA, 3989 bp, synthetic construct
  source 1..3989: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaaagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaacgcgtatcgatgatatcgcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaacgcgtgataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgagctctaatacgactcactataggacatttgcttctgacacaactgtgttcactagcaacctcaaacagacaccatggaggacgctaaaaatatcaagaagggtcccgctcctttctatccactcgaggacggtaccgctggtgagcaactgcacaaggctatgaaacggtatgctcttgtgccaggcaccatagctttcaccgacgcccacatagaggtggacattacttacgcagagtactttgagatgtctgtccggctggcagaggcaatgaaaagatatggcctcaacacaaaccataggatagtggtttgcagcgagaattctcttcagttttttatgcccgtcctcggggctcttttcatcggcgtggctgtcgcccccgccaacgacatatacaacgagcgagagctgctgaacagcatgggaatcagtcagcctacagtggtttttgtatcaaagaagggcttgcaaaagatccttaacgtacagaaaaagctccctattattcagaagatcattataatggattcaaagaccgactatcagggattccagtcaatgtatactttcgtcacttcccaccttcctcctgggtttaatgaatacgacttcgttcccgaaagtttcgatcgggacaaaactattgcactcatcatgaactcctctgggtccactgggctgcctaaaggtgtcgcacttccccacaggactgcatgtgtaaggttctctcacgcacgagatcccatattcggtaaccaaatcattcctgataccgctatcctctctgtagtgcctttccaccatggattcggcatgttcaccactctgggttacttgatatgtgggttccgcgtggtccttatgtacagatttgaagaggagctctttttgaggagcttgcaggactacaaaatccaaagtgctctcctcgtccccacattgttctcattcttcgccaaatctactctcatcgacaagtacgatttgagcaacttgcacgaaattgcttctgggggggctccactctccaaagaagtcggcgaagccgttgctaaaaggttccacttgccaggcattcgccaggggtatggccttactgagacaacctcagctattctcattacccctgagggtgacgataagcctggtgccgtcggtaaggtagtaccatttttcgaggcaaaggtggtcgacttggataccggaaaaaccctcggtgtgaatcagagaggcgaattgtgtgtcaggggtccaatgatcatgagcggttacgtcaataatcccgaggccactaacgccttgattgataaagatggttggcttcattcaggtgatatagcatactgggatgaggatgaacatttcttcattgtcgataggctcaaaagcctgatcaagtataagggttaccaagtggcccctgccgaactcgaatctatactgctgcaacatccaaacatttttgacgctggcgtggccggactgcccgatgatgatgctggtgaactccctgccgccgttgtcgtcctggaacatggtaagaccatgacagaaaaggagattgtagattacgtggcttcacaagtgactacagctaagaaattgcgcgggggtgtcgttttcgtggacgaggtacccaagggcctcactgggaagctcgacgccagaaaaatccgagaaatcctgattaaagcaaagaagggcggaaagattgcagtgtag

SEQ ID NO: 101 - DNA, 537 bp, synthetic construct
  source 1..537: mol_type = other DNA
atggacatgagagtcccagctcaactgcttggtcttcttctgctgtggttgcgcggtgccagatgtcacttcgggaagttggagtccaagttgagtgttattcgcaacctcaatgatcaggtgttgttcatagaccagggtaaccgcccactttttgaagatatgaccgatagcgattgccgcgacaacgcccccaggaccatcttcataataagtgcgtacggagactcacgggcccgggggaaggctgttacaatatccgtaaagtgcgagaagatctccacgctttcttgcgaaaataagattatcagttttaaagaaatgaaccctcctgacaacataaaagatacgaaatcagacatcatcttcttccagcgctcagttcctggacatgataataagatgcagttcgagtcatctagctatgaagggtattttctggcttgtgagaaagaacgggatttgtttaaacttatactgaagaaggaggatgaacttggtgatcggtctattatgtttaccgtacaaaatgaggac

SEQ ID NO: 102 - DNA, 537 bp, synthetic construct; identical to SEQ ID NO: 101
  source 1..537: mol_type = other DNA

SEQ ID NO: 103 - RNA, 550 nt, unidentified
  source 1..550: mol_type = other RNA
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataata

SEQ ID NO: 104 - RNA, 57 nt, synthetic construct
  source 1..57: mol_type = other RNA
gcgatcgcgctagcaacggtttccctctagcgggatcaattccgcccccccccccta

SEQ ID NO: 105 - RNA, 57 nt, synthetic construct; identical to SEQ ID NO: 104
  source 1..57: mol_type = other RNA

SEQ ID NO: 106 - DNA, 537 bp, synthetic construct
  source 1..537: mol_type = other DNA
atgacctcccggcttgtgagggtactggctgctgctatgctggtggctgctgctgtgagtgtggcacacttcggacgactccattgtactacagccgttatacgaaatatcaatgatcaggtgttgtttgtagacaaaaggcagcccgtgtttgaagacatgactgatattgatcagagtgcctctgagccccagactagactgattatatatgcttatggcgactctagagctagagggaaagcagtaactctgtcagtgaaagacagcaagatgtctaccctgtcttgcaagaataaaataatctcatttgaggagatggacccccctgaaaacatagatgacatccaaagtgacctgatattctttcagaagcgggtacccggtcataacaaaatggagttcgagtcaagcctctatgagggtcactttctcgcctgccaaaaggaagatgacgccttcaaattgatcttgaaaaaaaaagacgagaatggtgataagtctgtcatgttcacactcactaatttgcatcagtca

SEQ ID NO: 107 - DNA, 537 bp, Mus musculus
  source 1..537: mol_type = other DNA
atgacctcccggcttgtgagggtactggctgctgctatgctggtggctgctgctgtgagtgtggcaaactttgggagactccactgtaccactgccgttatacggaatataaacgatcaggtcctctttgtagacaagagacagccagttttcgaggacatgactgatatagatcagagtgcctctgagcctcaaactcggctcataatatacatgtataaggatagcgaggttcggggacttgccgtgactttgtccgtgaaagactcaaaaatgagcaccctgtcatgcaaaaataagataatttctttcgaggaaatggaccctcccgagaacattgatgatatccagtccgacttgatatttttccagaaacgggttcccggtcataacaaaatggagtttgagtcttcattgtacgagggtcattttttggcatgccagaaggaggatgatgccttcaaactgattttgaagaaaaaagacgagaacggagacaagtctgtcatgtttacacttaccaaccttcatcaatcc

SEQ ID NO: 108 - RNA, 3735 nt, Sin Nombre orthohantavirus
  source 1..3735: mol_type = other RNA
atgaatagaggaatatttaacatgctgagccgccgccccttccccgcccccacagctatgtggaggcctaggcgaaggaggcaggcggcaccactgcccgcccgtaatgggttggcatcccaaattcaacagctaaccacagctgtcagtgccctagtcattggtcaagccacccgacctcaacaaccaaggccacgcccgctaccacgccctaagaagcagtccccgaagcaaccaccgaatgctaaaaagacaaaaccgcaggagaacaaggagaaacaacccgcaaaaccgaagccgggtaagagacagcgtatggcactaaaattgatgctgacaggactctttgacgtgaacaacgaagccggtgacgtaataggacacgccctcgctatggaaggcaaagtgatgaagccacttcacgtgaaaggggtcatagaccacccggttttggcaaagctgaaatttaccaagtcatcagcgtacgacatggagttcgcccagcttccagccagtatgaaaagcgaagcatttgcatacaccagcgagcaccctgaaggcttctacaactggcatcatggtgccgtgcagtacagcggcggtcgctttacagtcccgcgcggagttgggggaaaaggcgacagtggaagaccgatcatggataatacaggtaaggtggtcgcgatagtgctgggcggcgctgacgaaggcgcccgcactgctctctccgtcgtcacttggaacagcaaaggaaagaccatcaaaacgactccggaagggactgaggaatggtctgcagcaccattagtcacggctatgtgcttctttgagaatgtatcgttcccgtgcagtcgtccgcctacgtgctattcccgaaaaccatctcgtgcccttgacatcctggaggagaacgtgaacaacgacgcctacgataccttattagcggcagtgttgaagtgcaggacgtccggtcggaacaaacgcagcatcaccgacgatttcaccctgaccagcccgtacctgggcacttgctcgtattgccatcatactgaaccctgcttcagcccaattaagatcgagcaagtttgggacgaggcggacgacggatcaatacgaatacagacatccgcccagttcgggtacgaccaaagtggggcagcagacgtcaacaagtacaggtacatgtcaattgaccaggaccatgccgtgaaagaaggctctatggatcatatcaagatcagcacatccgggccctgtaggcggcttcaatcacaaaggtacttcttgcttgcgaaatgcccaccgggagatagcgttacggttagcataaccggcggcactatggccacctcctgtacattagcacgtaaaataaggcaaaaattcgtaggacgtgagaagtatgaccttcccccagcacacggaaagaacatcccctgcaccgtgtatgatcgtcttaaggagacgtcagccggatacatcaccatgcacaggcctggacctcatgcctatacctcatacctagaggctacctcaggaaaaatttacgccaaaccaccatcaggaaagaacatcacgtacgagtgtaagtgtggcgattataaaactgcgaccgtttccgtcagaacagagatcgcaggatgtactgccataaaacagtgcgtggcgtacaagagcgaccagaccaagtgggtgttcaactcgcccgatctaatcaggcacgctgaccacacagcccaaggtaaattccactttcctttcaagcctaccttcagtacatgcctggtaccactggcacacgagccgactgtaattcatgggttcaaacacataagcctccatttagacacagaccacccaaccttgctcaccattagaagactgggcgagaaacccgaaccgacgtcggagtggatctcaggaaaaacagttaggaattttactgtcgacagagacgggctagagtatatatggggcaatcacgaaccggtgagagtatatgcacaagagtccgcccccggtgaccctcacggatggccacacaaaataattcaacactactatcaacgccacccagtatacactagcttggctgtagcagcttccgctgtggcggtgatagtcggcctggccaccgccgtgatgtgtatctgcagagcgcgacgggagtgcctaaccccgtacgcgttagccccgaatgcagtcatcccgacgtccttggccttattgtgctgcgttcggtccgccaacgccgaatctttcaccgacacgatgggatacctatggtcgaacagtcaaacatttttctgggtgcagctgtgcatccccttggcagtagtaatagtactcgtacgctgctgttcgtgctgcctcccttttttagtggttgcaggcgcctacctggcgaaggtagacgccttcgaacatgcgaccactgtcccaaatgtgccgaagattccgtataaagctttggtcgagagagccggatacgccccgcttaacttggaaattactgttatgtcttcagaggtcctcccttcgattcaccaagaatacatcacctgcaaatactccactatcgtgccttccccgaaagtgaaatgctgcggttcccttgagtgccaacccgcttcgcatgccgactacaactgcaaagtatttggcggcgtttaccctttcatgtggggtggagcgcaatgtttttgcgacagcgagaacagccagatgagtgaggcgtatgtcgagctatcggccgattgtgcccttgatcacgcacaggcggtgaaagtgcacacagccgcaatgaaggtcggactacgcattgtgtatgggaacaccactagcctcctggatgcgtacgtaaacggagtcacgccaggcacatctaaagacctgaaagtcattgccggacccatctcgtccgcgttttccccttttgactctaaagtcgtgatacaccgagggctcgtgtacaattacgattttccagagtacggggcaatgaaaccgggtgcctttggggacattcaagctagctctttgacgggcaacgacctgattgccagcaccgacatacgtttgcttaaacccgcagcaaagaatgtgcacgttccctatacccaagcagcatcaggatttgaaatttggaagaacaactccggaagaccactgcaagagacggccccatttggctgtaagattgccgtcaaccctctgaaggccgtggattgttcctatgggaacatcccgatatcgattgatatcccccacgccgcctttattagaccatccgatgcgccatttatctccgaagtgaagtgcgaagtcagcgactgcacatactctgctgacttcggaggcatggccaccctacagtatgtgtctgaccgagagggccagtgcccagtgcattcccattcaagtaccgccacactccaggaatcaactgttcacgtcttggaaaaaggggcgacgacagtgcacttcagcacagctagccctcacgccagctttgtggtctcattatgcggaaagaagacgacctgcaccgcaggatgcaaaccacctgtcggccacatagtcagcacaccccacaagaaggaccaagagttccagaccgccgtctccaggacatcatggagctggctcttcgcgctgttcggaggagcttcgtccctagtaatcatgggactgttaatcttctcctgcagcatgatgctcactaacaccagaaga